(19) United States
(12) Patent Application Publication
(10) Pub. No.: US 2023/0027026 A1
(43) Pub. Date: Jan. 26, 2023

(54) FUSED PYRIMIDINE COMPOUNDS, COMPOSITIONS AND MEDICINAL APPLICATIONS THEREOF

(71) Applicants: JUBILANT BIOSYS LIMITED, Bangalore, Karnataka (IN); LENGO THERAPEUTICS, INC., Menlo Park, CA (US)

(72) Inventors: Chandregowda Venkateshappa, Bangalore (IN); Dhanalakshmi Sivanandhan, Bangalore (IN); Sridharan Rajagopal, Bangalore (IN); Bruce Roth, Menlo Park, CA (US); Anjali Pandey, Menlo Park, CA (US); Tracy Saxton, Menlo Park, CA (US); Gurulingappa Hallur, Bangalore (IN); Naveena Madhyastha, Bangalore (IN); Naveen Sadhu M, Bangalore (IN)

(21) Appl. No.: 17/764,092

(22) PCT Filed: Sep. 26, 2020
(86) PCT No.: PCT/US2020/052953

§ 371 Date: Mar. 25, 2022

(30) Foreign Application Priority Data
Sep. 27, 2019 (IN) 201941039277

(51) Int. Cl. A61K 31/519; A61K 31/5377

(52) U.S. Cl. CPC A61K 31/519; A61K 31/5377

(57) ABSTRACT

The present disclosure relates to a class of fused pyrimidine compounds of Formula I, their stereoisomers, tautomers, pharmaceutically acceptable salts, polymorphs, solvates, and hydrates thereof. The present disclosure also relates to a process of preparation of these fused pyrimidine compounds, and to pharmaceutical compositions containing them.

### CROSS-REFERENCE

This application claims the benefit of Indian patent application number 201941039277 filed on Sep. 27, 2019, which application is incorporated herein by reference.

### BACKGROUND OF THE INVENTION

Lung cancer accounts for the greatest number of cancer deaths, and approximately 85% of lung cancer cases are non-small cell lung cancer (NSCLC). The development of targeted therapies for lung cancer has primarily focused on tumors displaying specific oncogenic drivers, namely mutations in epidermal growth factor receptor (EGFR) and anaplastic lymphoma kinase (ALK). Three generations of tyrosine kinase inhibitors (TKIs) have been developed for cancers with the most frequently observed EGFR mutations, however, other oncogenic drivers in the EGFR family of receptor tyrosine kinases have received less research and development focus and several oncogenic drivers, including insertions in the exon 20 gene of EGFR, have no currently approved therapeutics to treat their cancers.

The mutation, amplification and/or overexpression of human epidermal growth factor receptor 2 (HER2), another member of the human epidermal growth factor receptor family of receptor tyrosine kinases, has been implicated in the oncogenesis of several cancers, including lung, breast, ovarian, and gastric cancers. Although targeted therapies such as trastuzumab and lapatinib have shown clinical efficacy especially in breast tumors, their utility in lung cancer has been limited. It is likely that this variation is due to tissue-specific factors, including the low potency of kinase inhibitors like lapatinib for the mutagenic alterations in HER2 that are observed in the lung cancer patient population, including insertions in the exon 20 gene of HER2.

Given that many patients with mutations in EGFR and HER2 do not derive clinical benefit from currently available therapies against these targets, there remains a significant unmet need for the development of novel therapies for the treatment of cancers associated with EGFR and HER2 mutations.

### SUMMARY OF THE INVENTION

In one aspect, provided herein is a compound of Formula I.

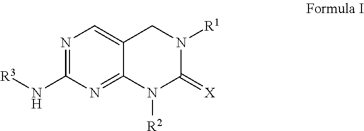

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

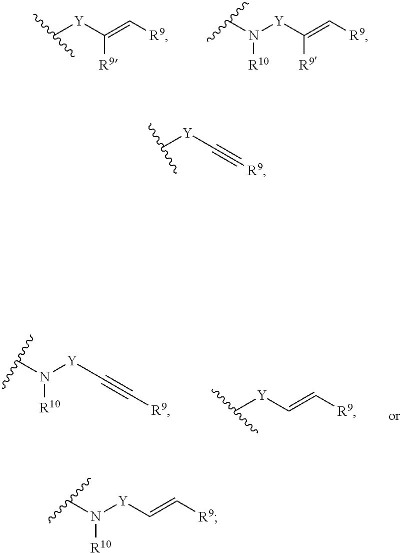

Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

In another aspect, provided herein is a compound of Formula I:

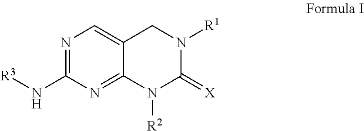

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with at least one R⁷ and 0, 1, or 2 R⁸;
   each R⁷ is independently or

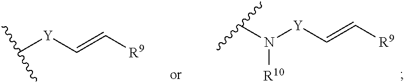

Y is —C(═O)—, —S(═O)—, or —S(═O)₂—;
R⁹ is hydrogen, halo, alkyl, cycloalkyl, or heteroalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

In some embodiments, n is 0 or 1.

In some embodiments, R⁵ is phenyl, naphthyl, anthracenyl, phenanthrenyl, chrysenyl, pyrenyl, C-linked pyridyl, C-linked pyrimidinyl, C-linked pyrazolyl, or C-linked imidazolyl. In some embodiments, R⁵ is unsubstituted. In some embodiments, R⁵ is substituted with 1 or 2 R^{5′}.

In some embodiments, each R⁴ is independently hydrogen, alkyl, halo, haloalkyl, or alkoxy. In some embodiments, each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy. In some embodiments, each R⁴ is independently hydrogen, methyl, fluoro, trifluoromethyl, methoxy, or trifluoromethoxy.

In some embodiments, each R^{5′} is independently aryl, heteroaryl, alkyl, heterocycloalkyl, halo, cyano, hydroxy, —N(R⁶)₂, or alkoxy. In some embodiments, each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy. In some embodiments, each R^{5′} is independently phenyl, imidazolyl, pyridinyl, methyl, tert-butyl, pyrrolidinyl, morpholinyl, fluoro, cyano, hydroxy, —N(R⁶)₂, or methoxy.

In some embodiments, each R⁶ is independently alkyl or aryl. In some embodiments, each R⁶ is independently methyl, ethyl, iso-propyl, tert-butyl, phenyl, or naphthyl. In some embodiments, each R⁶ is independently methyl or phenyl.

In some embodiments, X is S. In some embodiments, X is O.

In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl. In some embodiments, R² is phenyl, cyclohexyl, or pyrrolyl.

In some embodiments, R⁷ is

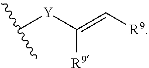

In some embodiments, R⁷ is

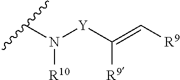

In some embodiments, R⁷ is

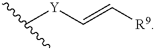

In some embodiments, R⁷ is

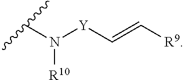

In some embodiments, Y is —C(═O)—. In some embodiments, Y is —S(═O)₂—.

In some embodiments, R⁹ and R^{9′} are independently hydrogen, halo, alkyl, heteroalkyl, haloalkyl, or (alkyl)heterocycloalkyl. In some embodiments, R⁹ is hydrogen, halo, or heteroalkyl. In some embodiments, R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl. In some embodiments, R⁹ is hydrogen, fluoro, chloro, hydroxyethyl, or methoxyethyl.

In some embodiments, R¹⁰ is hydrogen, methyl, ethyl n-propyl, iso-propyl, n-butyl, sec-butyl, tert-butyl, trifluoromethyl, or cyclopropyl. In some embodiments, R¹⁰ is hydrogen or methyl.

In some embodiments, R² is substituted with 1 or 2 R⁸.

In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano.

In some embodiments, each R¹¹ is independently alkyl or aryl. In some embodiments, each R¹¹ is independently methyl, ethyl, n-propyl, iso-propyl, n-butyl, iso-butyl, sec-butyl, tert-butyl, phenyl, naphthyl, anthracenyl, or phenanthrenyl. In some embodiments, each R¹¹ is independently methyl, ethyl, iso-propyl, tert-butyl, phenyl, or naphthyl. In some embodiments, each R¹¹ is independently methyl or phenyl.

In some embodiments, R² is unsubstituted.

In some embodiments, R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl. In some embodiments, R³ is imidazolyl, triazolyl, indolyl, indazolyl, thiazolyl, isothiazolyl, or pyridinyl.

In some embodiments, R³ is selected from:

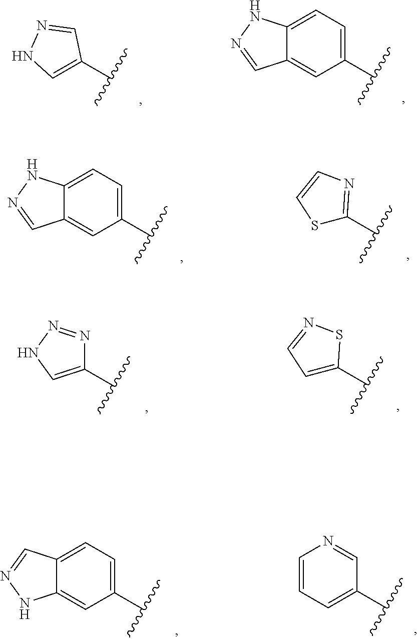

wherein R³ is substituted with 0 to 3 R¹².

In some embodiments, R³ is selected from:

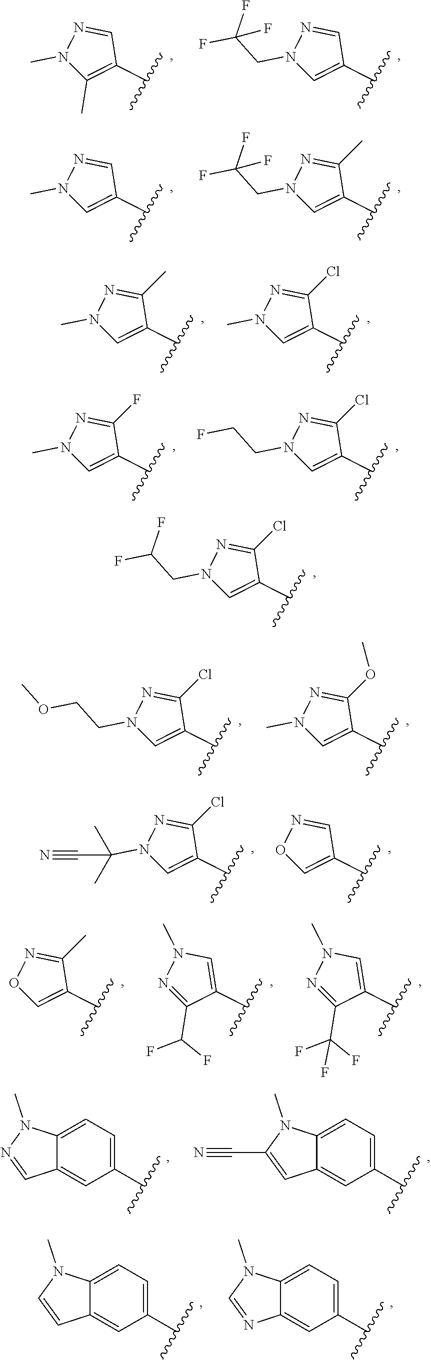

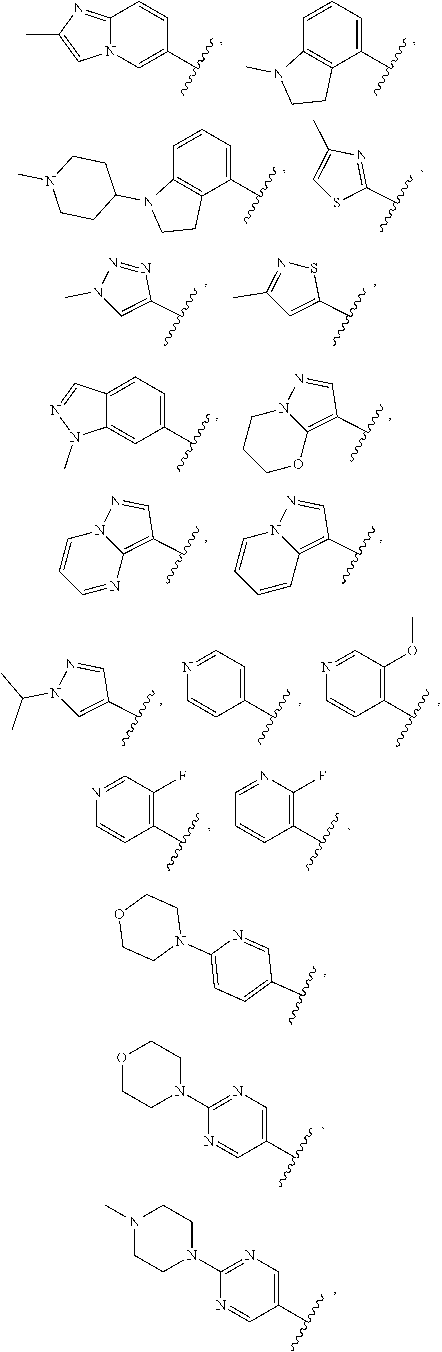

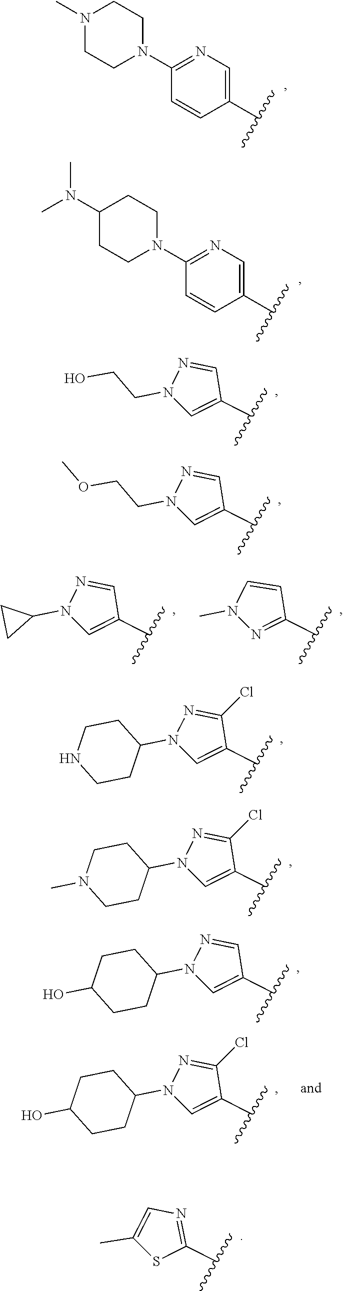

In some embodiments, R³ is selected from:

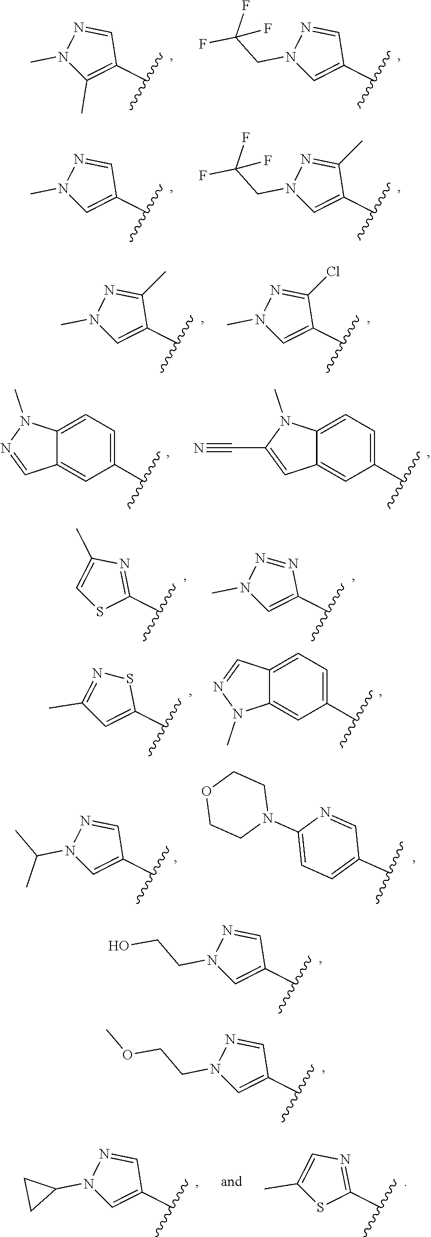

In some embodiments, R³ is unsubstituted. In some embodiments, R³ is substituted with at least 1 R¹². In some embodiments, R³ is substituted with at least 2 R¹².

In some embodiments, each R¹² is independently aryl, heteroaryl, alkyl, heteroalkyl, haloalkyl, halo, cyano, heterocycloalkyl, —N(R¹³)₂, —S(═O)₂NH₂, or cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, chloro, cyano, morpholinyl, or cyclopropyl. In some embodiments, each R¹² is independently methyl, hydroxyethyl, methoxyethyl, trifluoroethyl, or chloro. In some embodiments, each R¹² is independently methyl or chloro.

In some embodiments, each R¹³ is independently alkyl or cycloalkyl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl.

In some embodiments, the aryl, heteroaryl, heterocycloalkyl, or cycloalkyl of R¹² is unsubstituted. In some embodiments, the aryl, heteroaryl, heterocycloalkyl, or cycloalkyl of R¹² is substituted with 1 or 2 R¹⁴.

In some embodiments, each R¹⁴ is independently alkyl, cycloalkyl, heterocycloalkyl, halo, cyano, —N(R¹⁵)₂, or alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂ methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹⁵)₂, or methoxy.

In some embodiments, each R¹⁵ is independently alkyl or cycloalkyl. In some embodiments, each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl.

In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl.

In some embodiments the compound of Formula I is selected from:

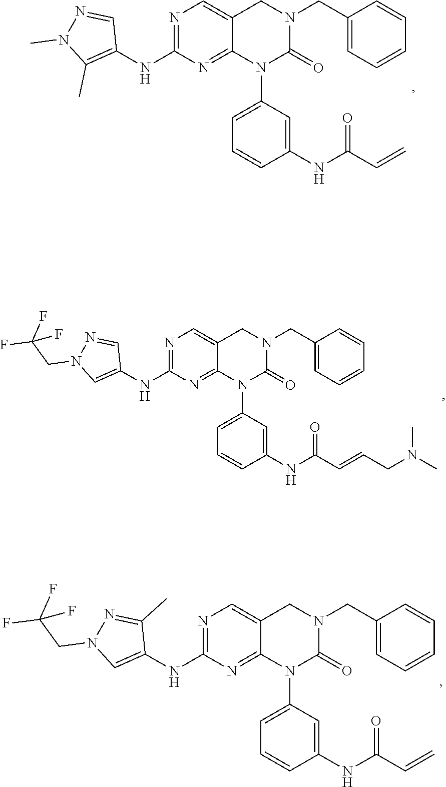 [SMILES: C=CC(=O)Nc1cccc(N2C(=O)N(Cc3ccccc3)Cc3cnc(Nc4cnn(C)c4C)nc32)c1 ; "," ; CN(C)C/C=C/C(=O)Nc1cccc(N2C(=O)N(Cc3ccccc3)Cc3cnc(Nc4cnn(CC(F)(F)F)c4)nc32)c1 ; "," ; C=CC(=O)Nc1cccc(N2C(=O)N(Cc3ccccc3)Cc3cnc(Nc4cn(CC(F)(F)F)nc4C)nc32)c1 ; ","]

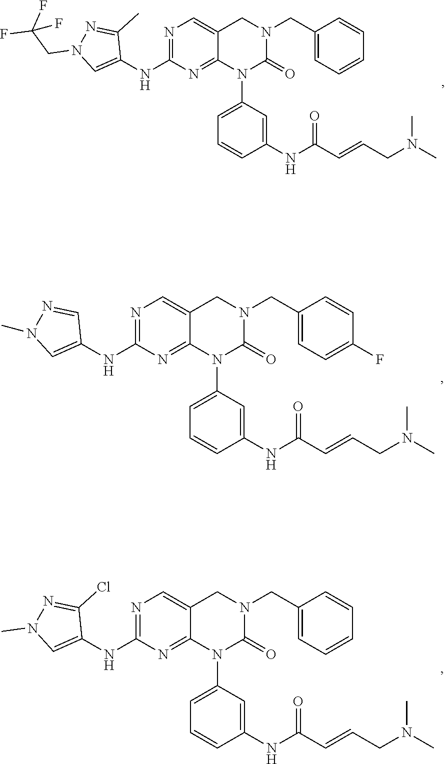

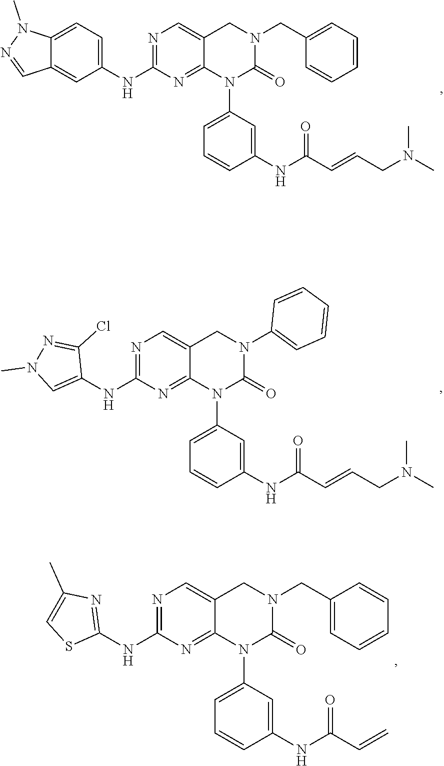

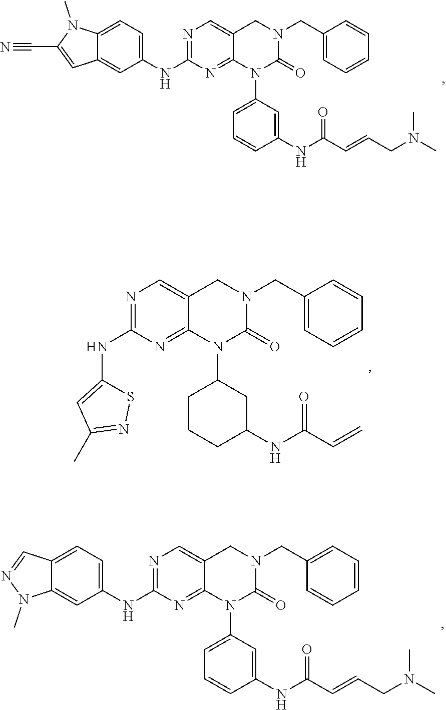

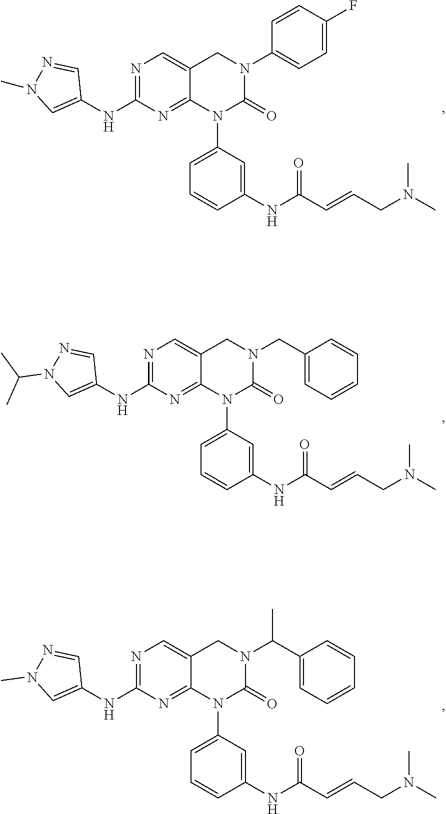

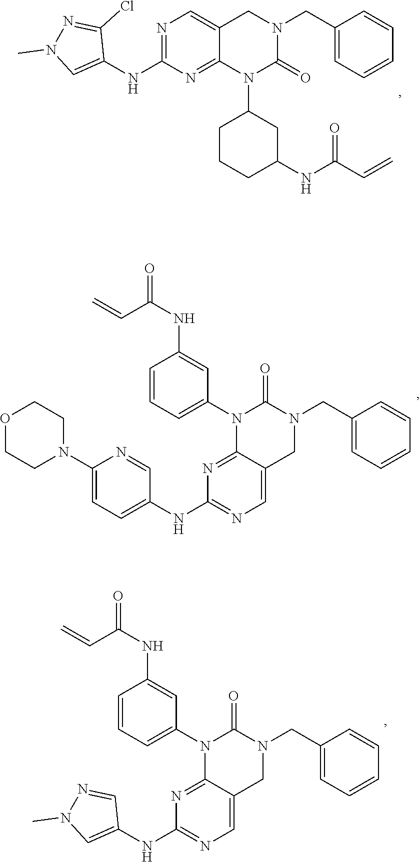 [SMILES: C=CC(=O)NC1CCCC(N2C(=O)N(Cc3ccccc3)Cc3cnc(Nc4cn(C)nc4Cl)nc32)C1 ; "," ; C=CC(=O)Nc1cccc(N2C(=O)N(Cc3ccccc3)Cc3cnc(Nc4ccc(N5CCOCC5)nc4)nc32)c1 ; "," ; C=CC(=O)Nc1cccc(N2C(=O)N(Cc3ccccc3)Cc3cnc(Nc4cnn(C)c4)nc32)c1 ; ","]

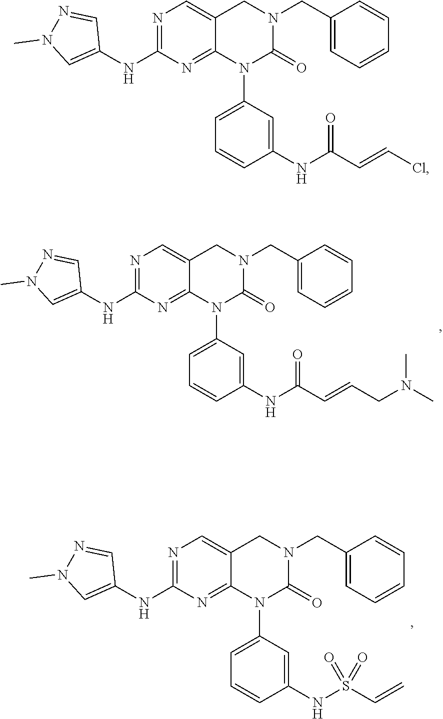

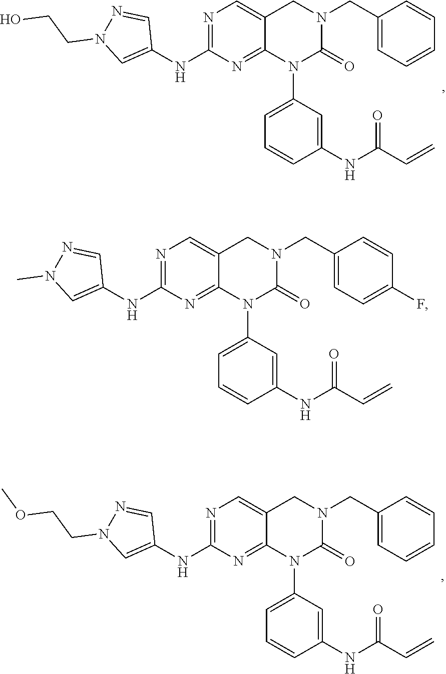 [SMILES: C=CC(=O)Nc1cccc(N2C(=O)N(Cc3ccccc3)Cc3cnc(Nc4cnn(CCO)c4)nc32)c1 ; "," ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; ","]

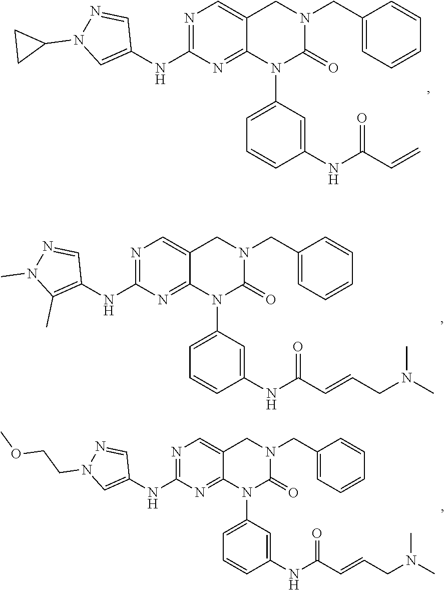

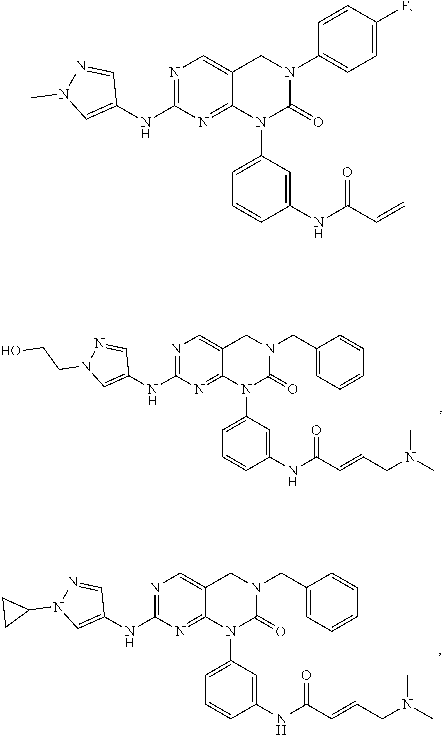 [SMILES: C=CC(=O)Nc1cccc(N2C(=O)N(c3ccc(F)cc3)Cc3cnc(Nc4cnn(C)c4)nc32)c1 ; CN(C)C/C=C/C(=O)Nc1cccc(N2C(=O)N(Cc3ccccc3)Cc3cnc(Nc4cnn(CCO)c4)nc32)c1 ; "," ; CN(C)C/C=C/C(=O)Nc1cccc(N2C(=O)N(Cc3ccccc3)Cc3cnc(Nc4cnn(C5CC5)c4)nc32)c1 ; ","]

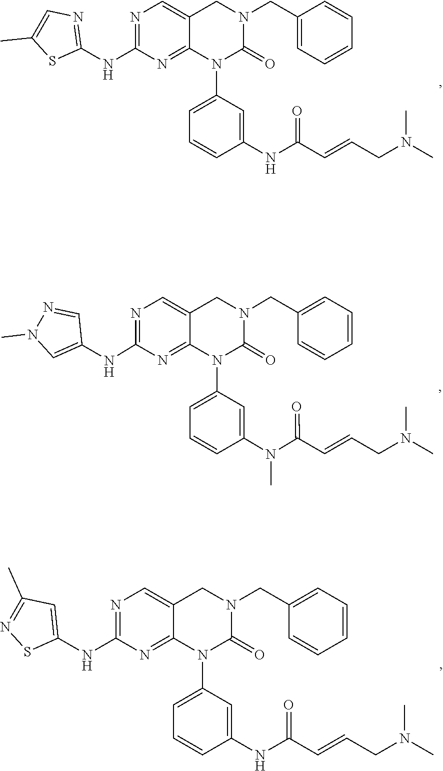

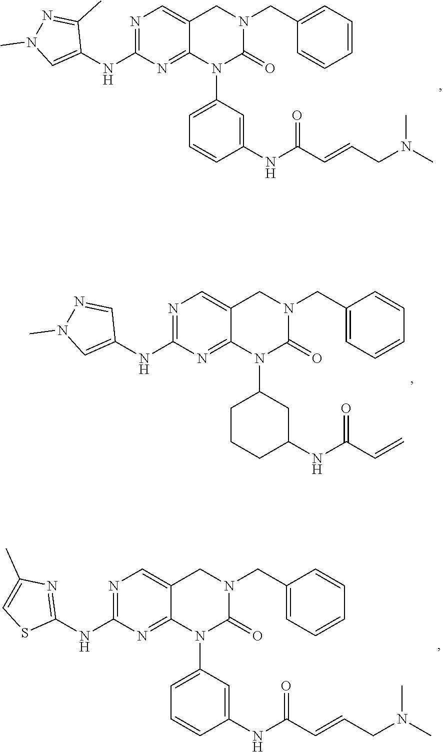

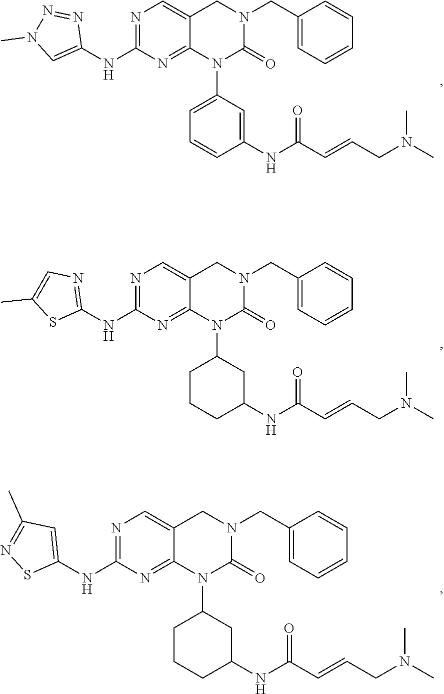

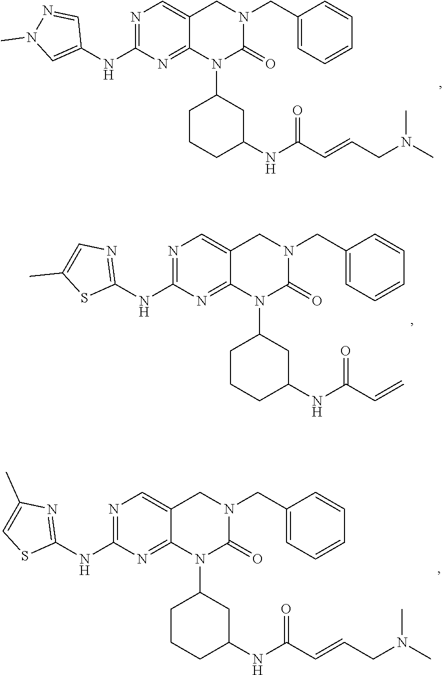

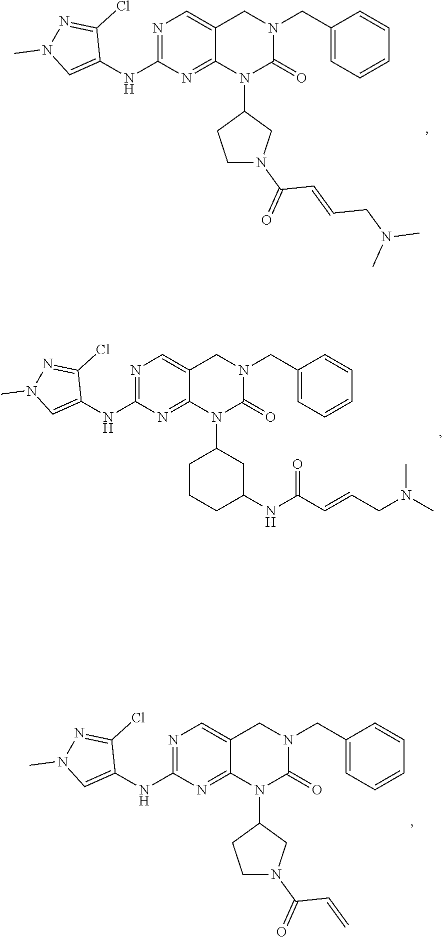

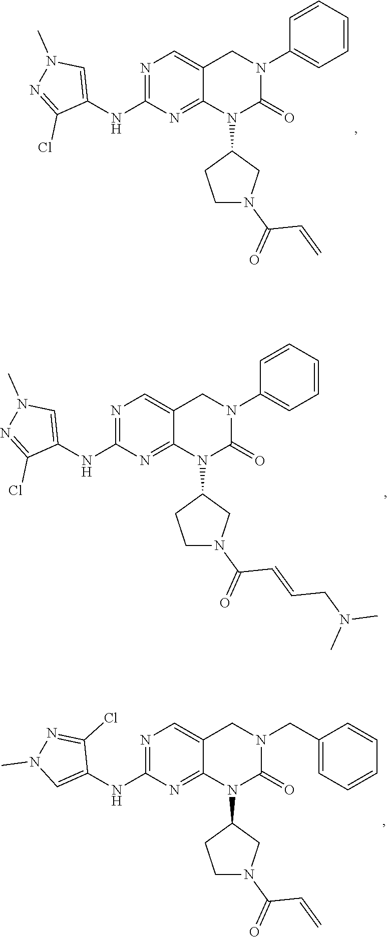

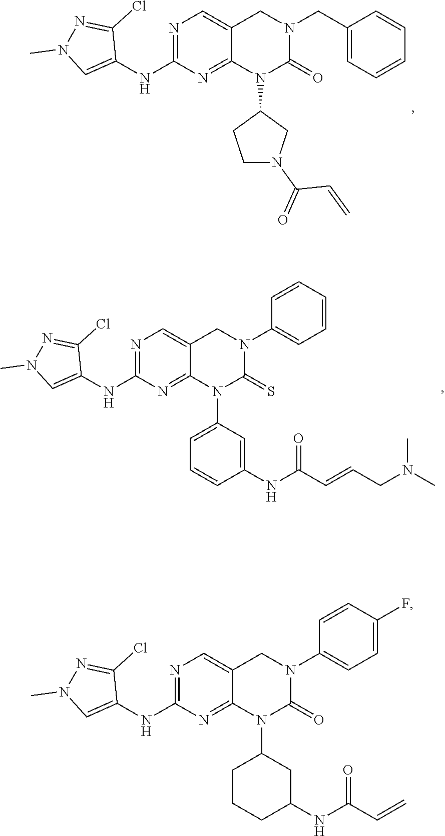

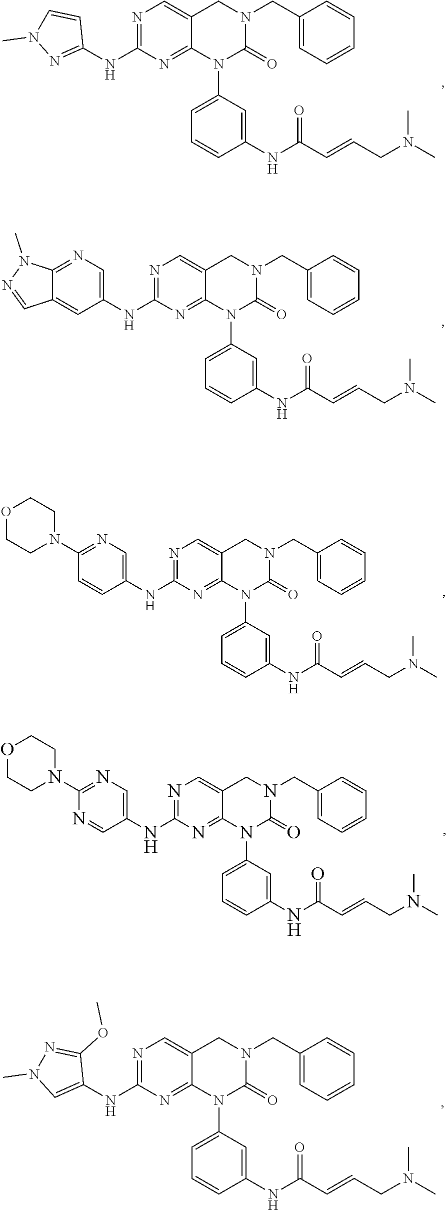

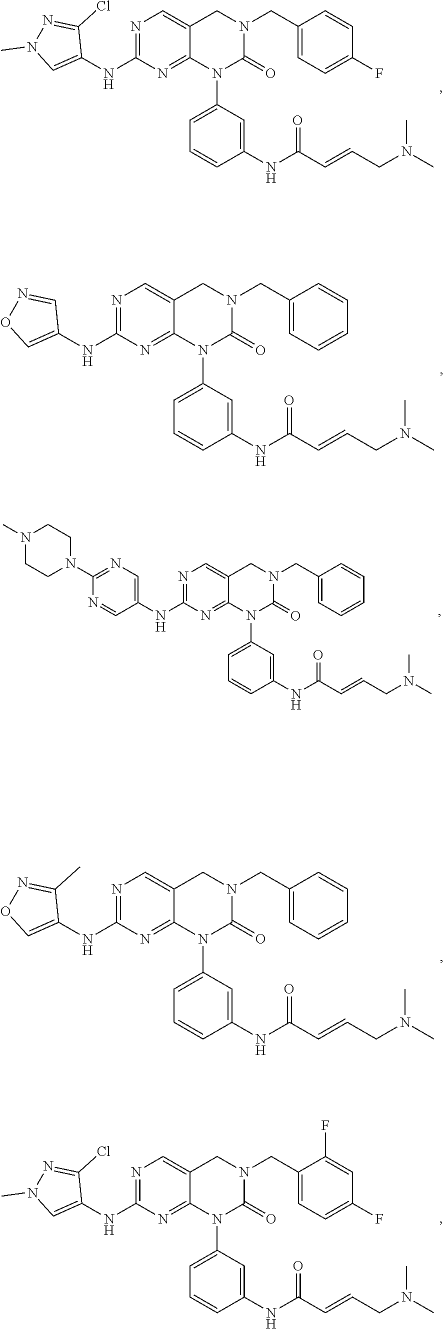

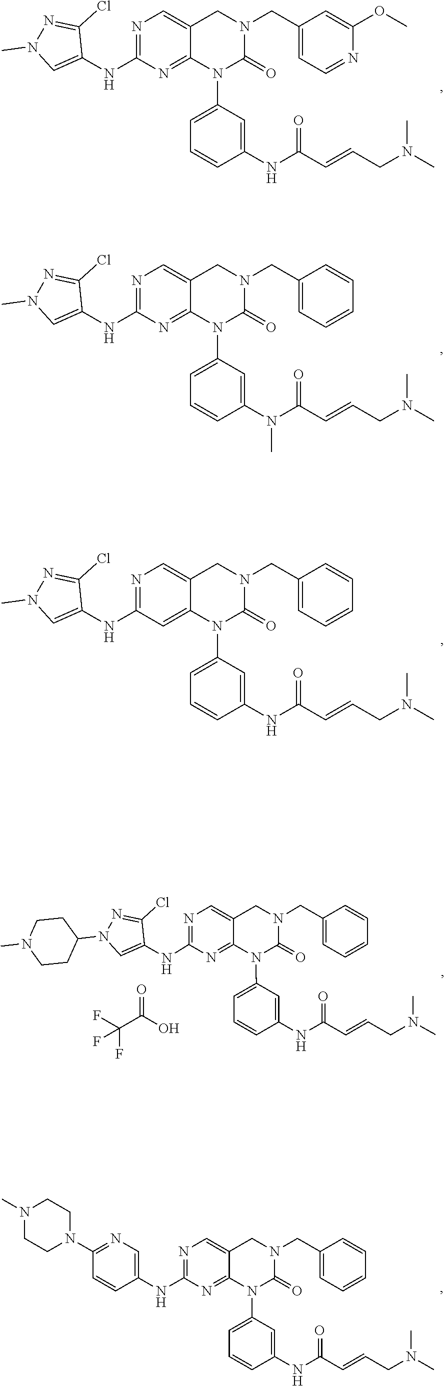

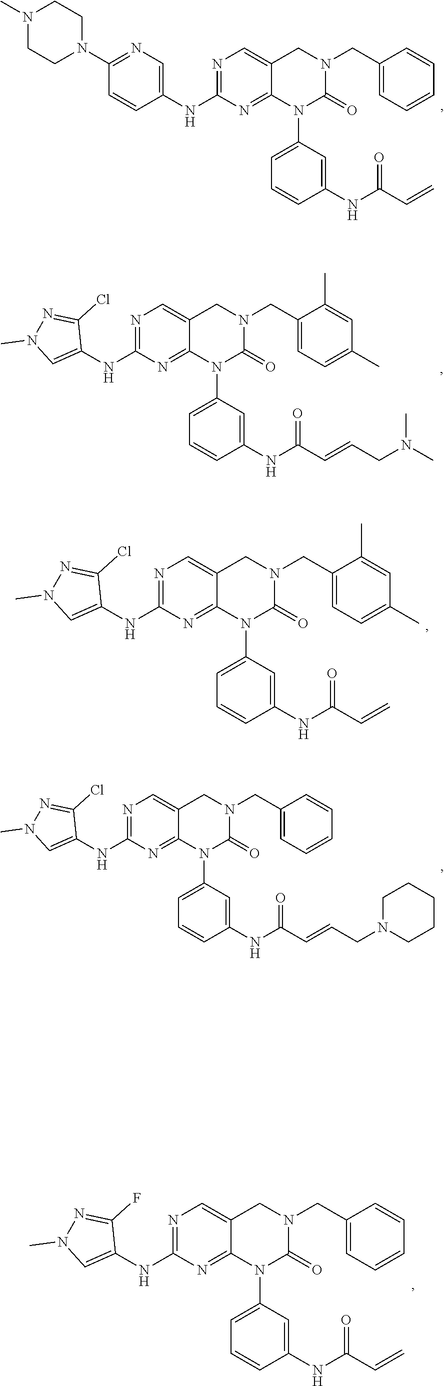

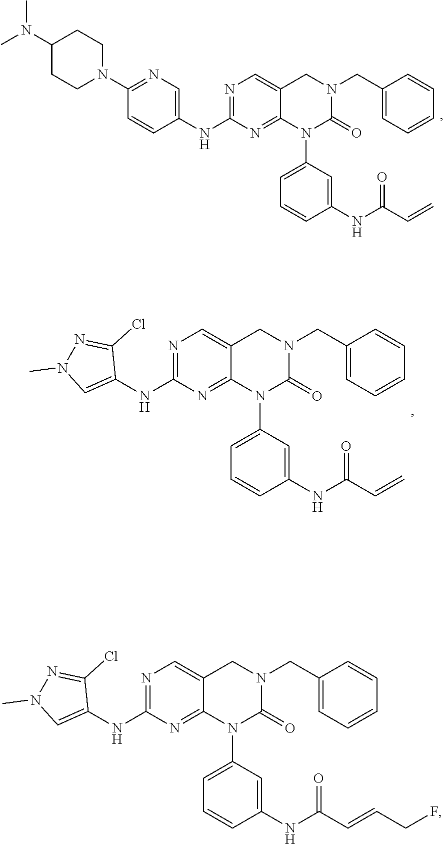 [SMILES: C=CC(=O)Nc1cccc(N2C(=O)N(Cc3ccccc3)Cc3cnc(Nc4ccc(N5CCC(N(C)C)CC5)nc4)nc32)c1 ; "," ; C=CC(=O)Nc1cccc(N2C(=O)N(Cc3ccccc3)Cc3cnc(Nc4cn(C)nc4Cl)nc32)c1 ; "," ; Cn1cc(Nc2ncc3c(n2)N(c2cccc(NC(=O)/C=C/CF)c2)C(=O)N(Cc2ccccc2)C3)c(Cl)n1]

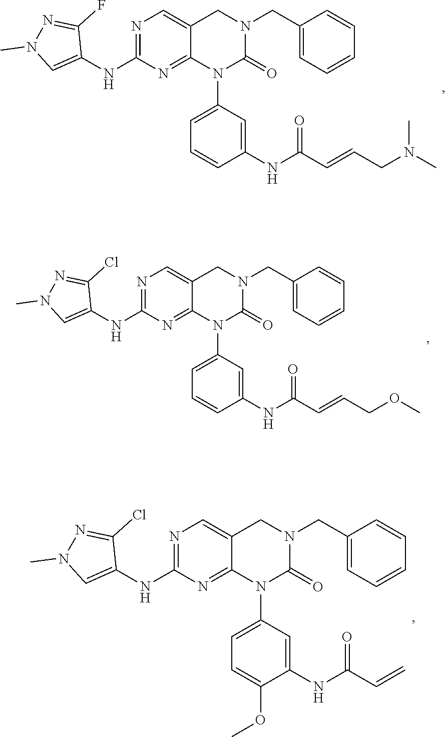

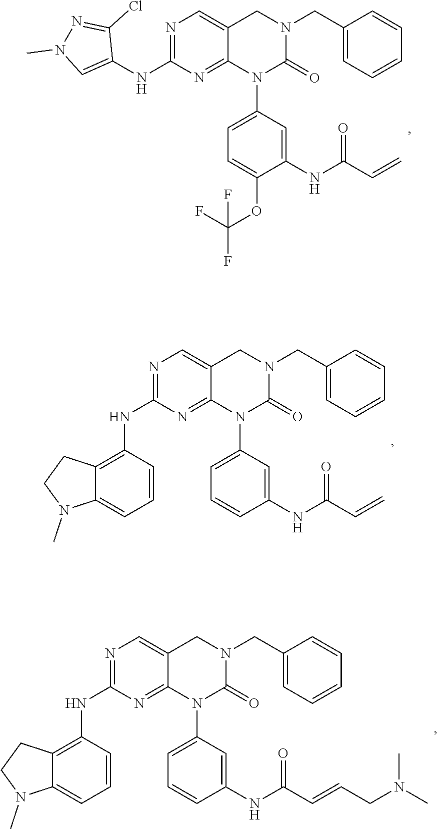 [SMILES: C=CC(=O)Nc1cc(N2C(=O)N(Cc3ccccc3)Cc3cnc(Nc4cn(C)nc4Cl)nc32)ccc1OC(F)(F)F ; "," ; C=CC(=O)Nc1cccc(N2C(=O)N(Cc3ccccc3)Cc3cnc(Nc4cccc5c4CCN5C)nc32)c1 ; "," ; CN(C)C/C=C/C(=O)Nc1cccc(N2C(=O)N(Cc3ccccc3)Cc3cnc(Nc4cccc5c4CCN5C)nc32)c1 ; ","]

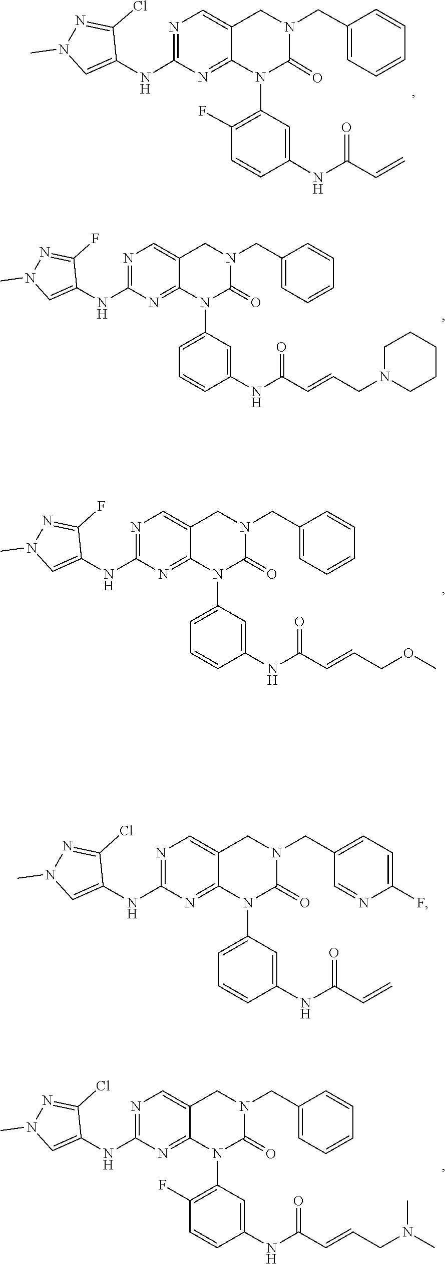

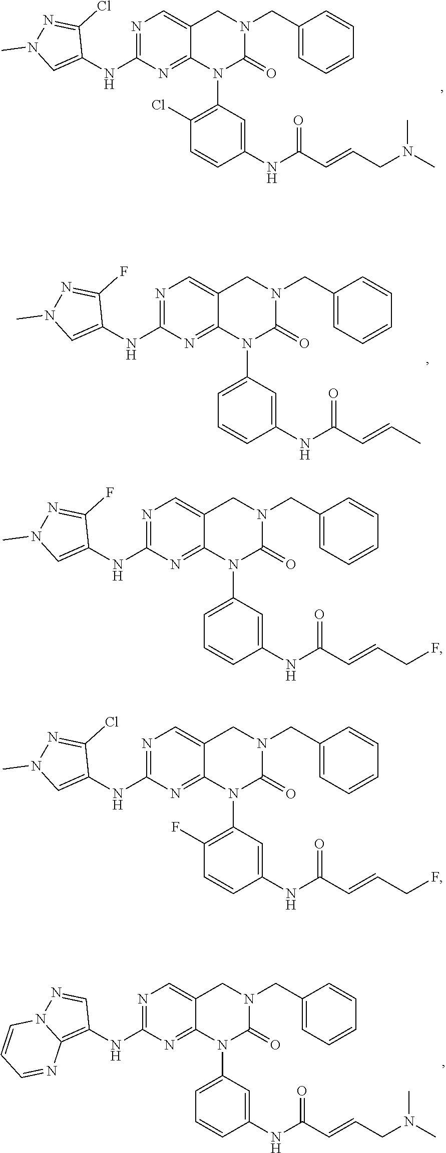

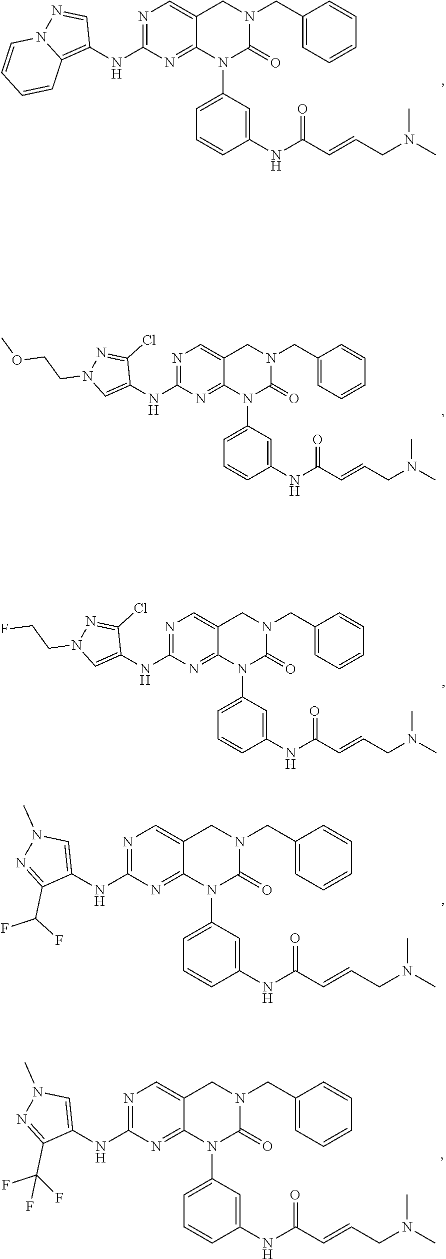

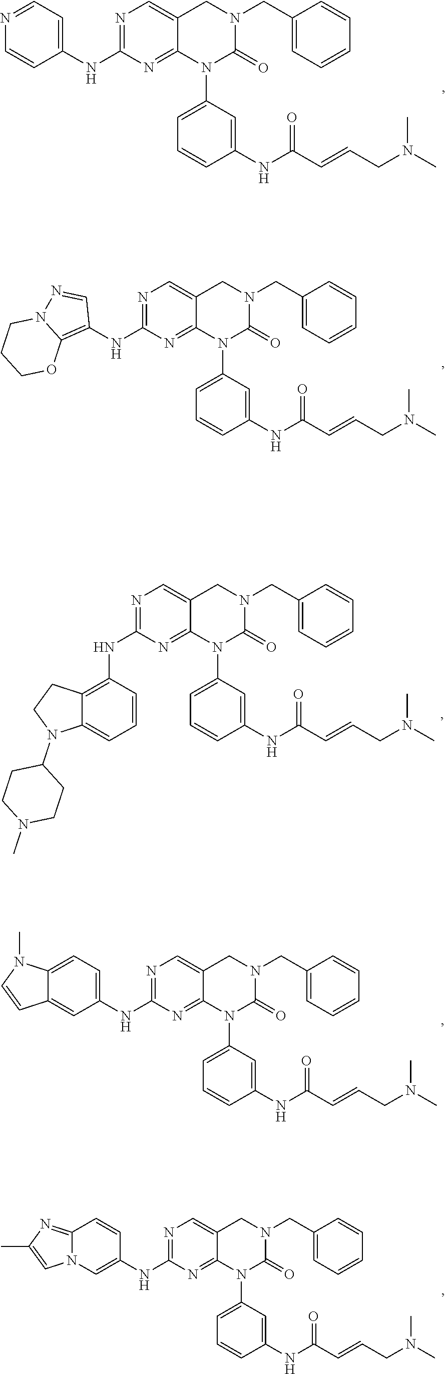

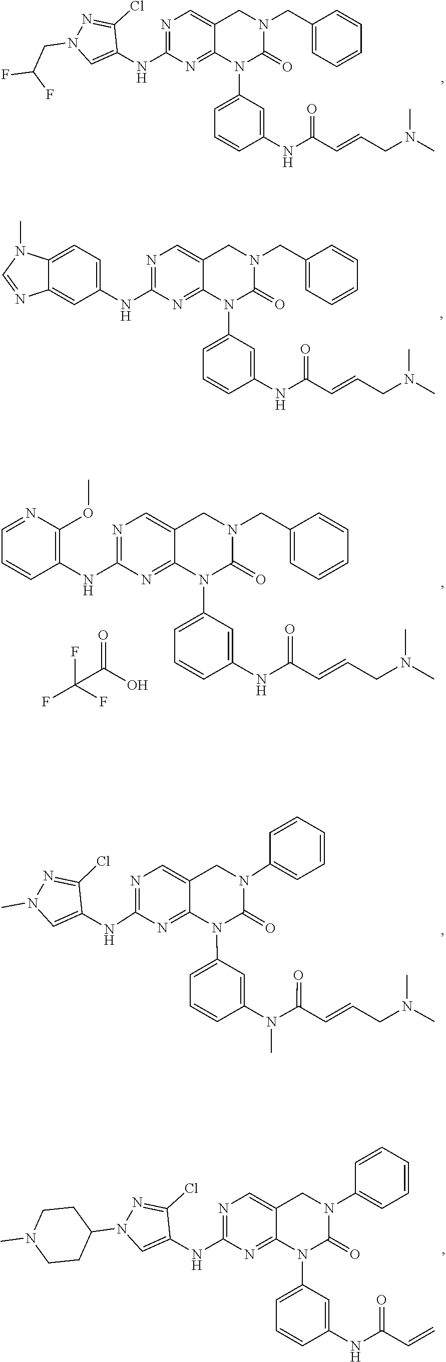

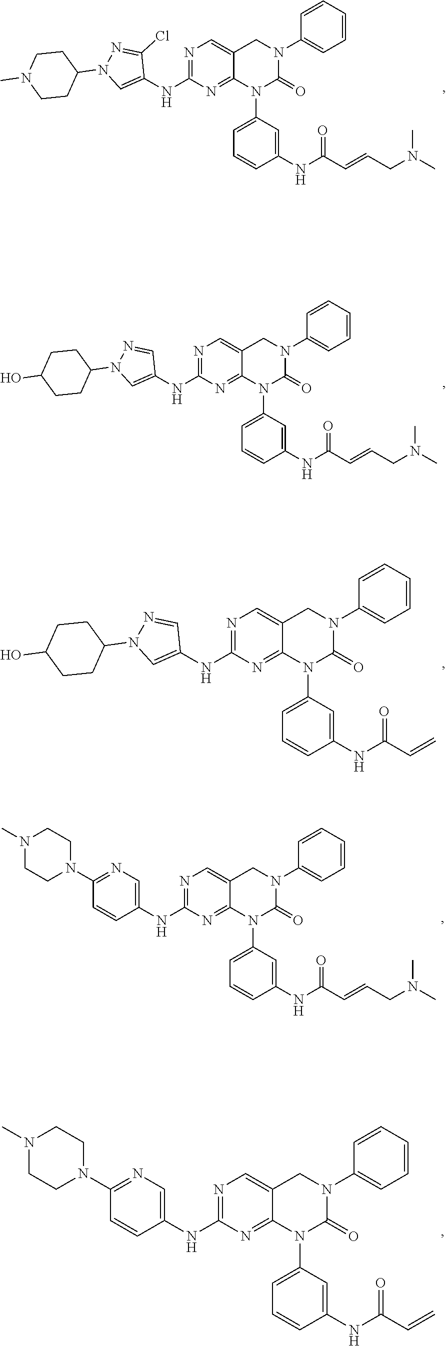

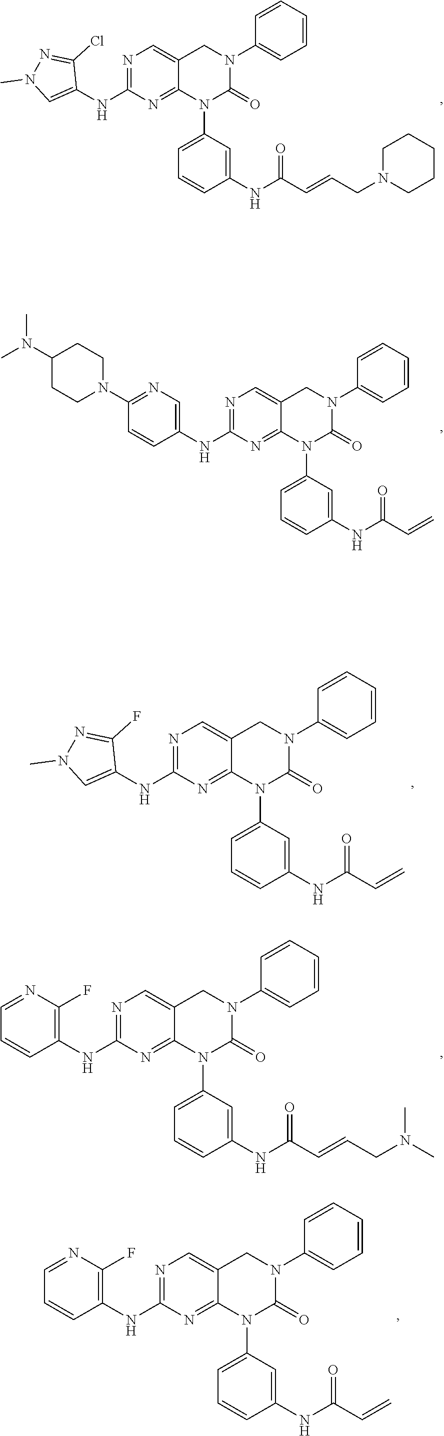

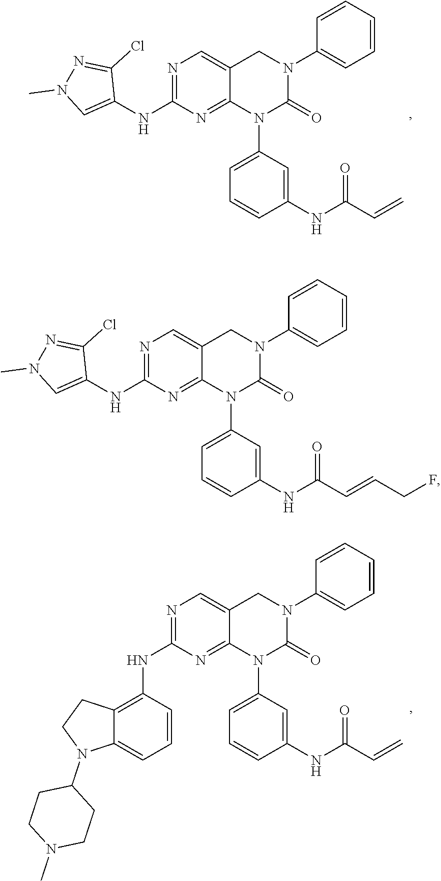

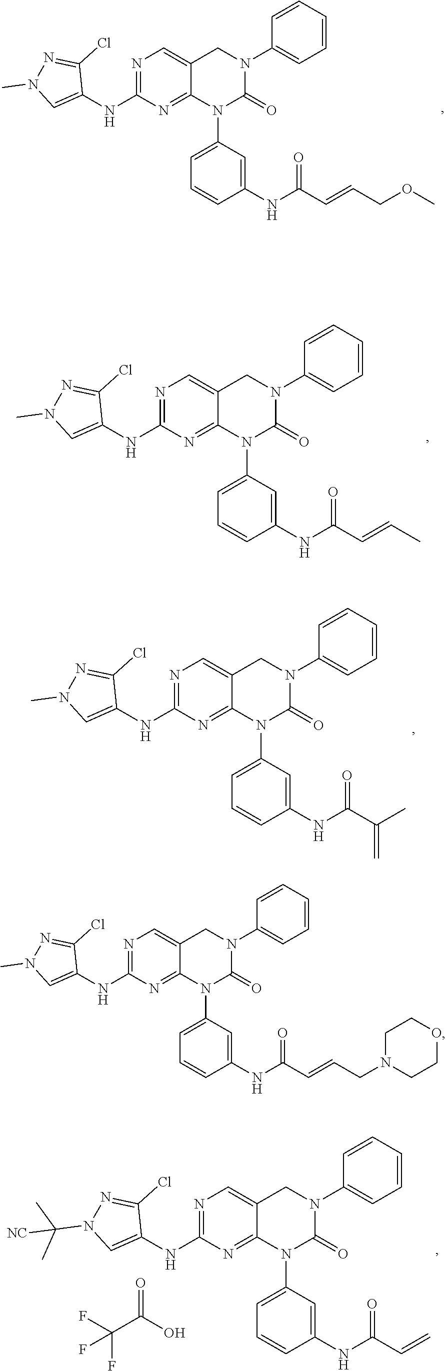

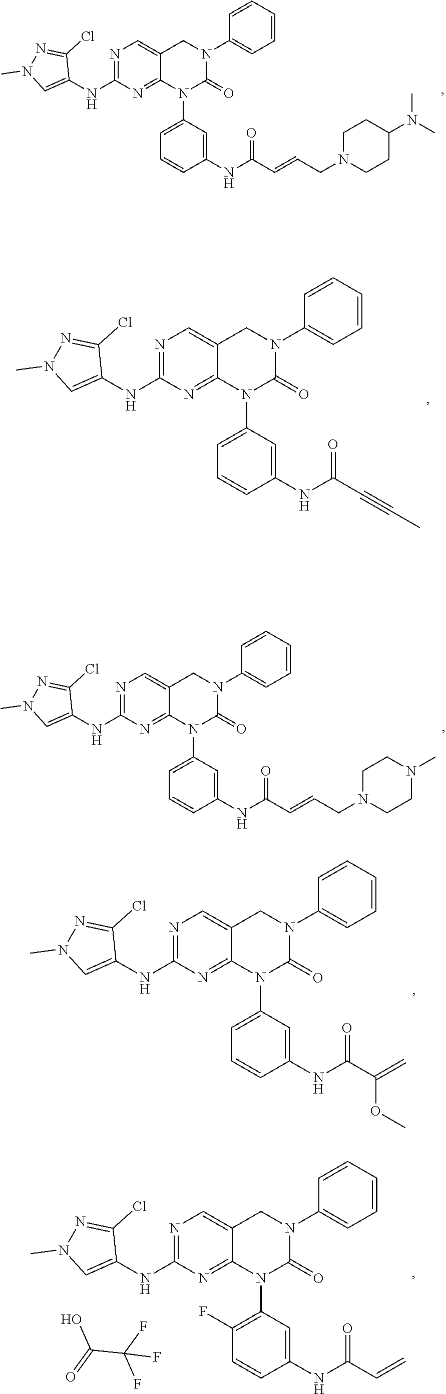

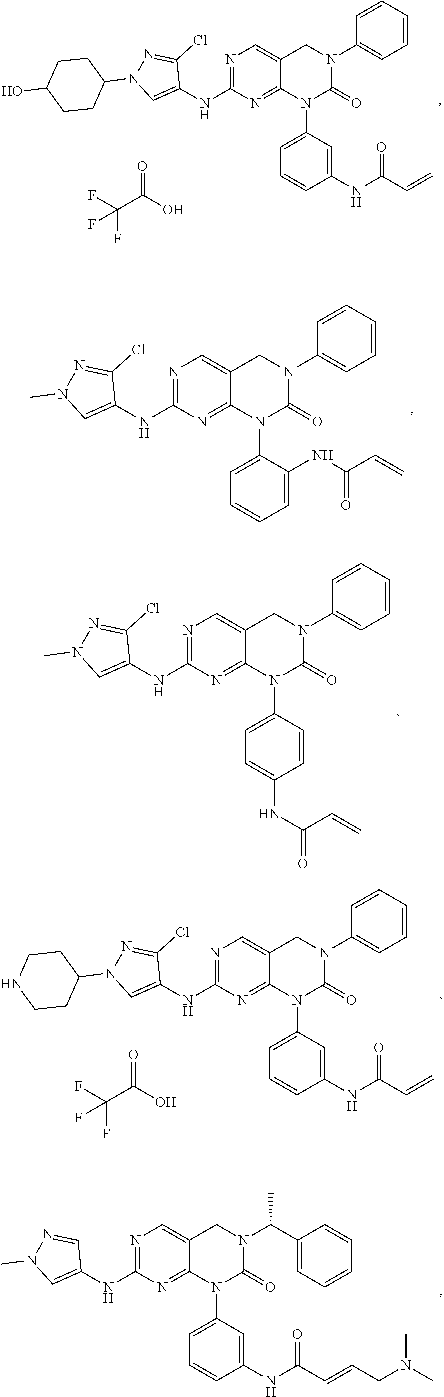

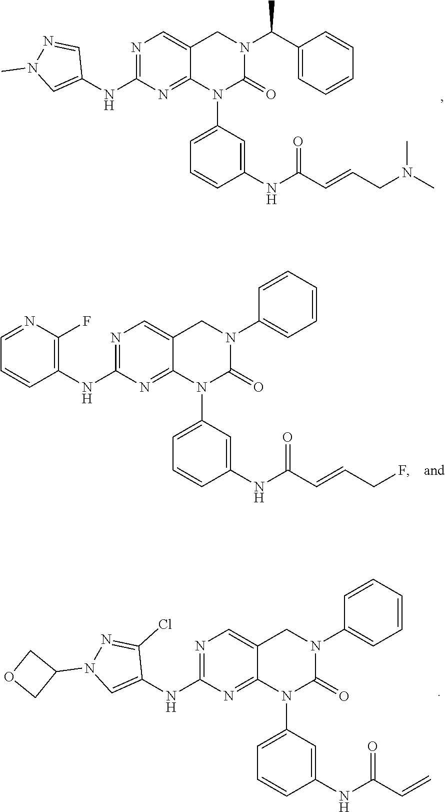

In some embodiments, the compound is selected from:

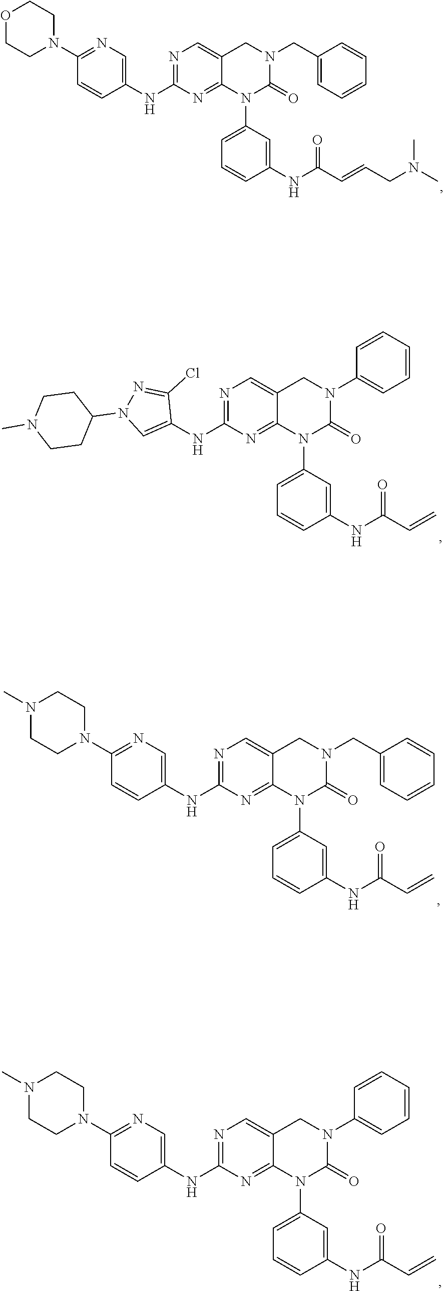

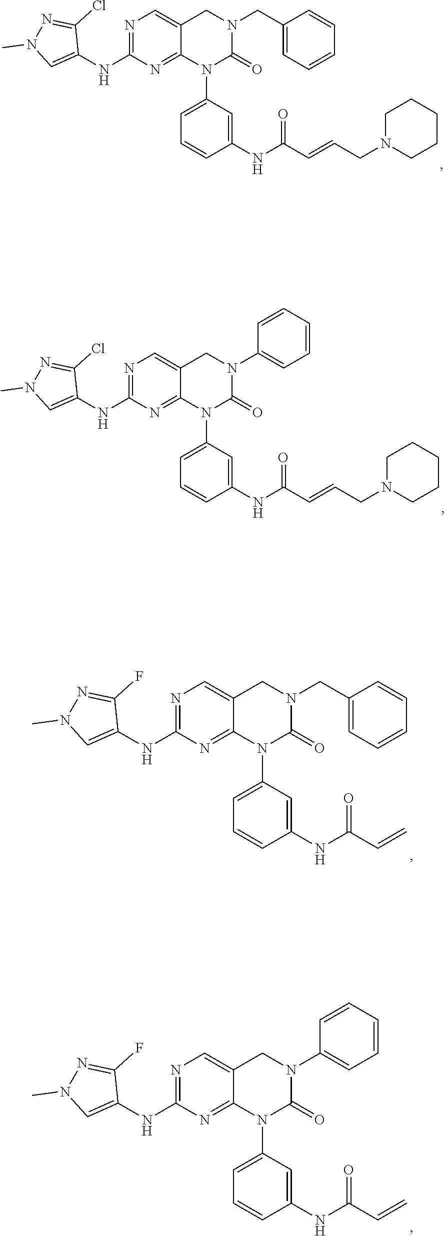 [SMILES: Cn1cc(Nc2ncc3c(n2)N(c2cccc(NC(=O)/C=C/CN4CCCCC4)c2)C(=O)N(Cc2ccccc2)C3)c(Cl)n1 ; Cn1cc(Nc2ncc3c(n2)N(c2cccc(NC(=O)/C=C/CN4CCCCC4)c2)C(=O)N(c2ccccc2)C3)c(Cl)n1 ; C=CC(=O)Nc1cccc(N2C(=O)N(Cc3ccccc3)Cc3cnc(Nc4cn(C)nc4F)nc32)c1 ; C=CC(=O)Nc1cccc(N2C(=O)N(c3ccccc3)Cc3cnc(Nc4cn(C)nc4F)nc32)c1]

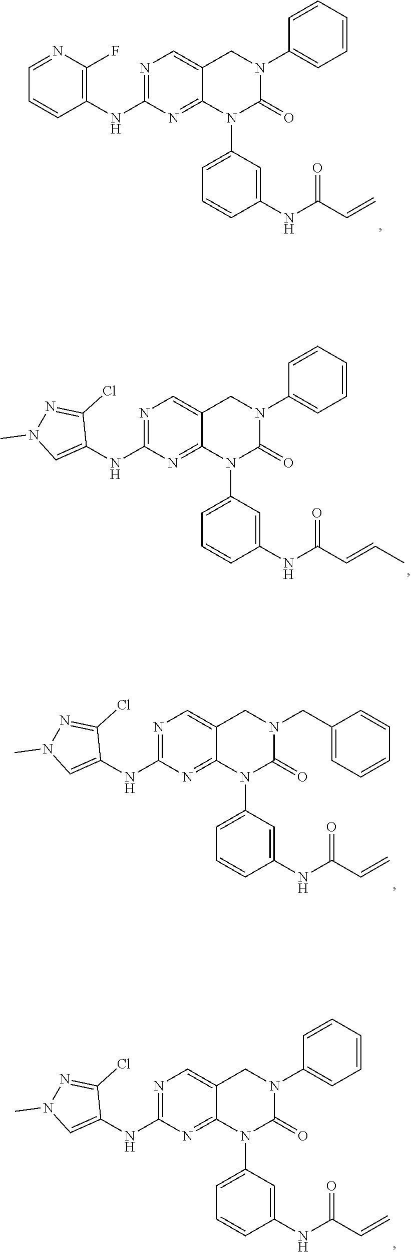

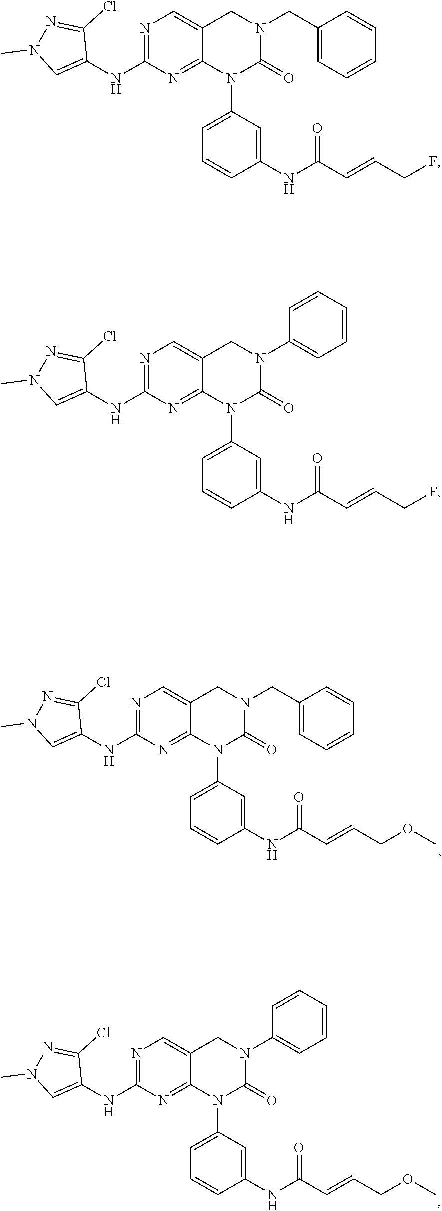

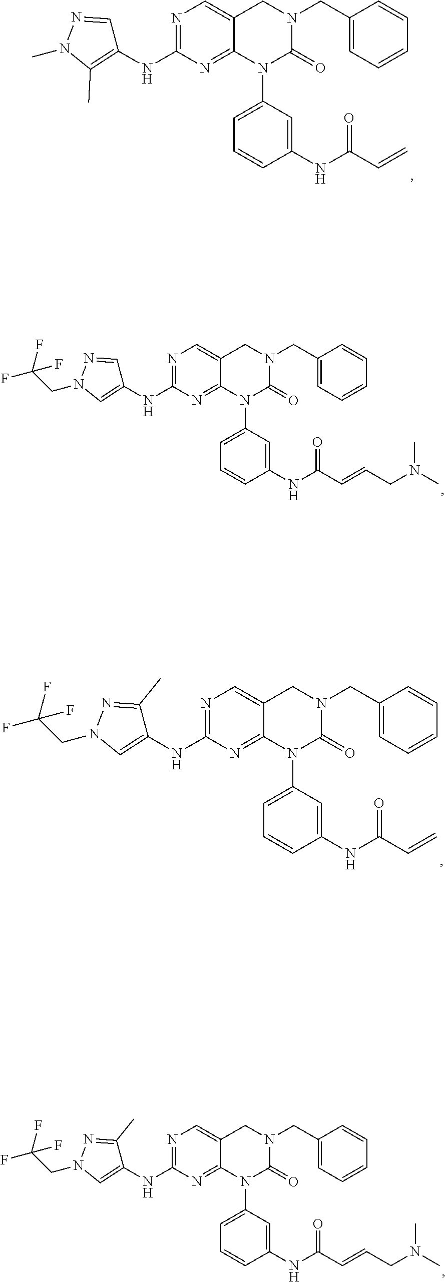 [SMILES: C=CC(=O)Nc1cccc(N2C(=O)N(Cc3ccccc3)Cc3cnc(Nc4cnn(C)c4C)nc32)c1 ; CN(C)C/C=C/C(=O)Nc1cccc(N2C(=O)N(Cc3ccccc3)Cc3cnc(Nc4cnn(CC(F)(F)F)c4)nc32)c1 ; C=CC(=O)Nc1cccc(N2C(=O)N(Cc3ccccc3)Cc3cnc(Nc4cn(CC(F)(F)F)nc4C)nc32)c1 ; Cc1nn(CC(F)(F)F)cc1Nc1ncc2c(n1)N(c1cccc(NC(=O)/C=C/CN(C)C)c1)C(=O)N(Cc1ccccc1)C2]

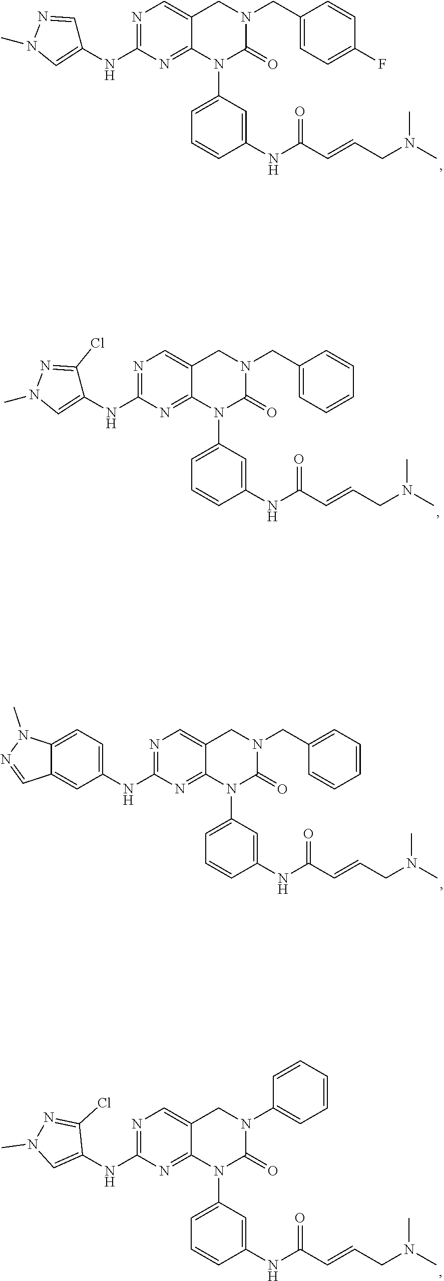

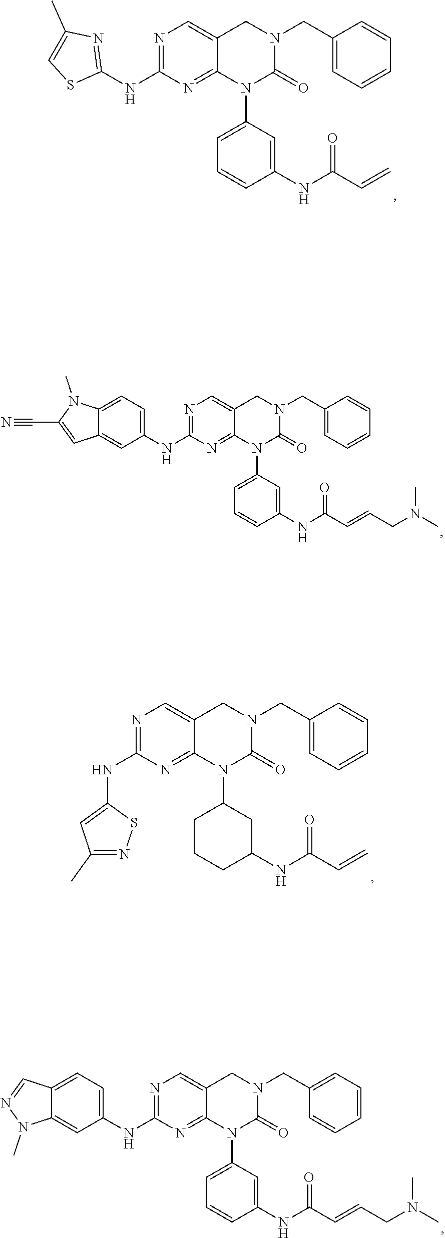

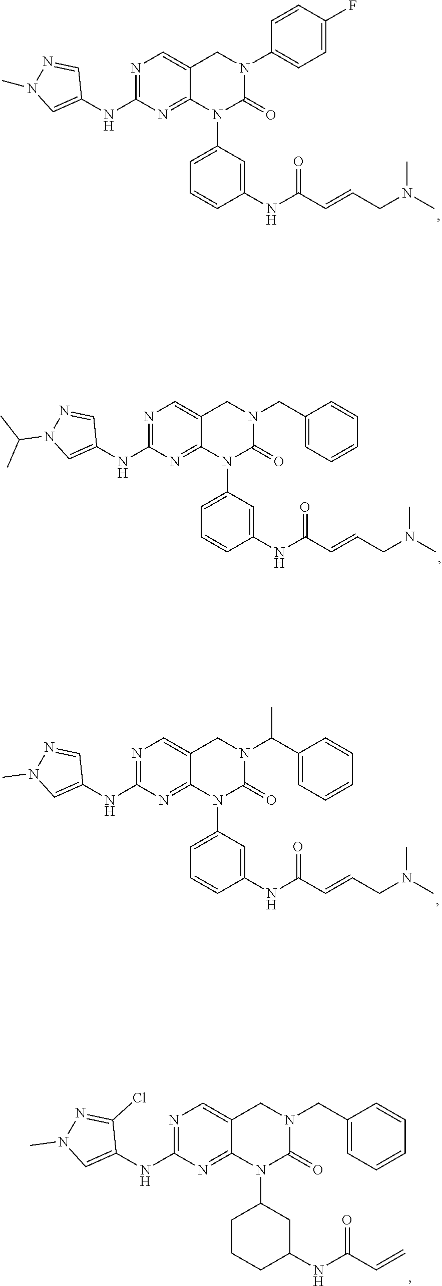

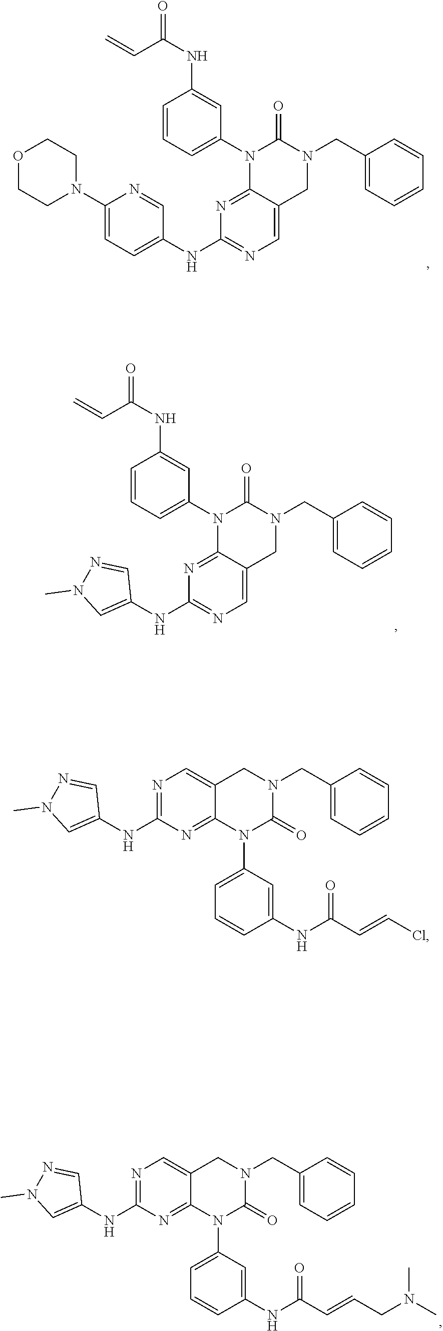

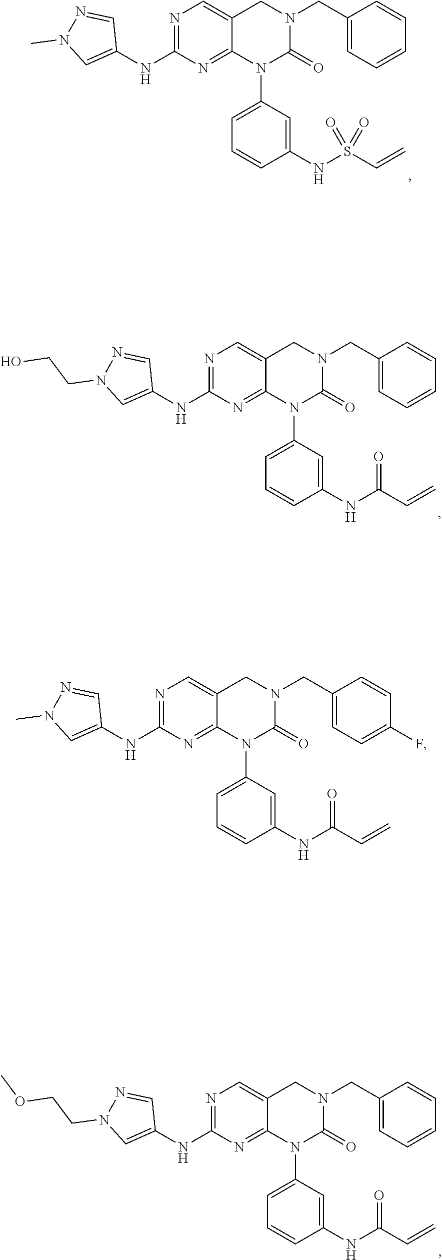

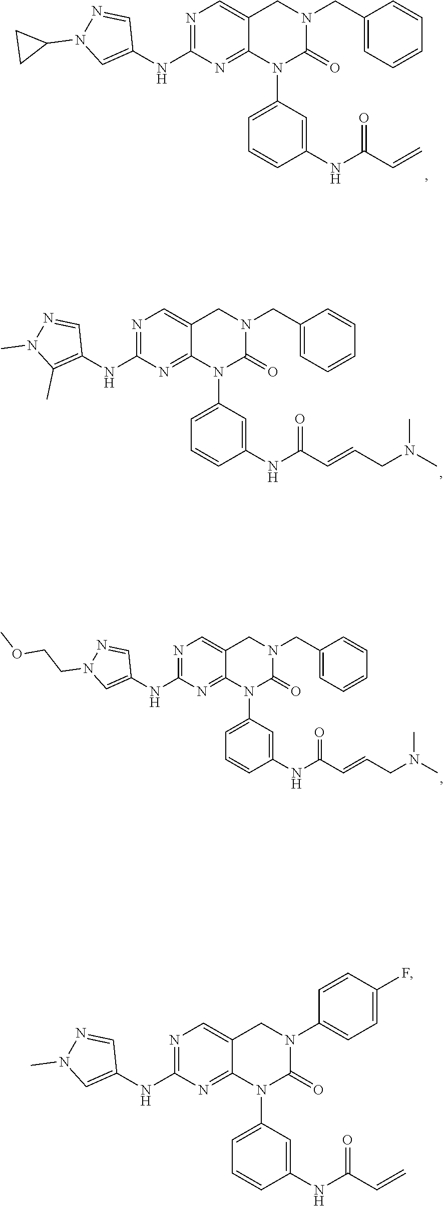

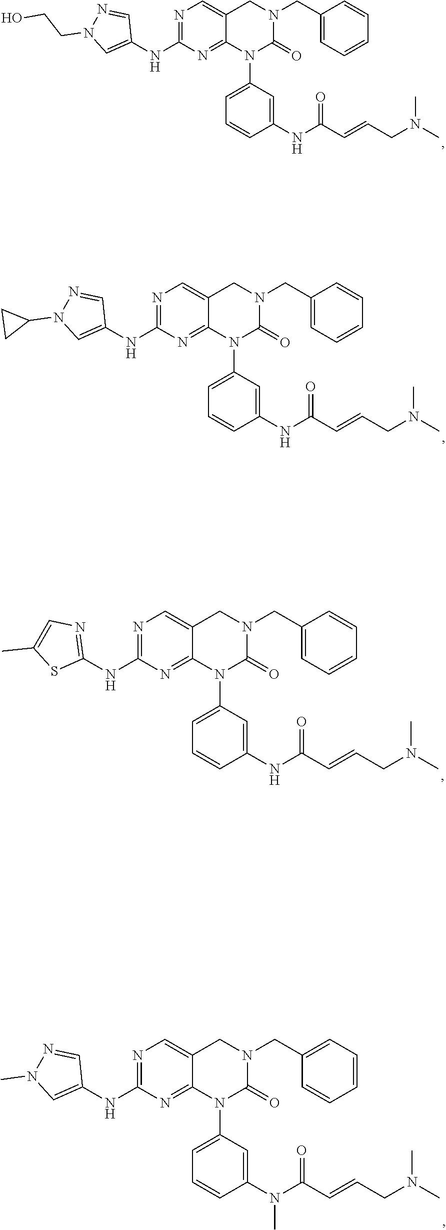

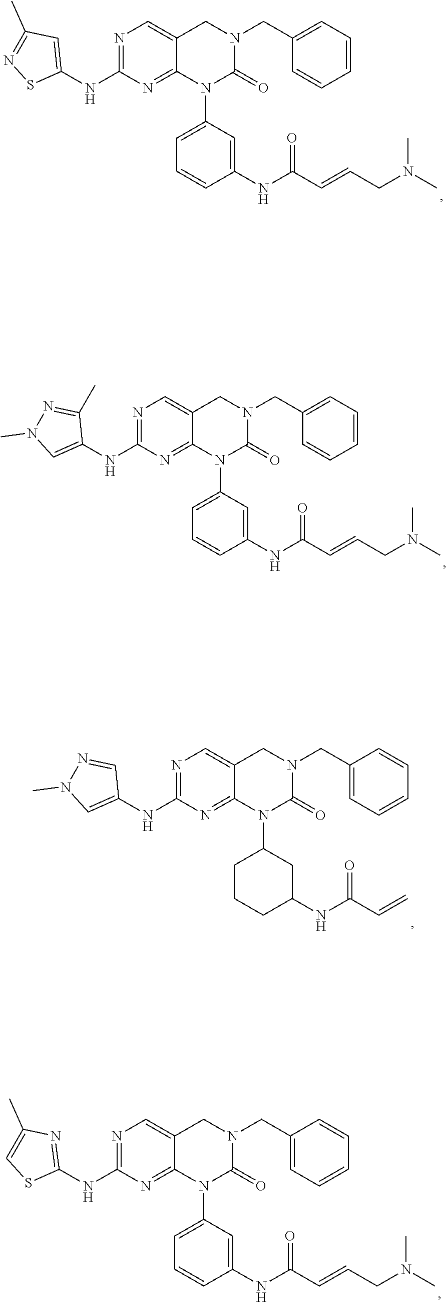

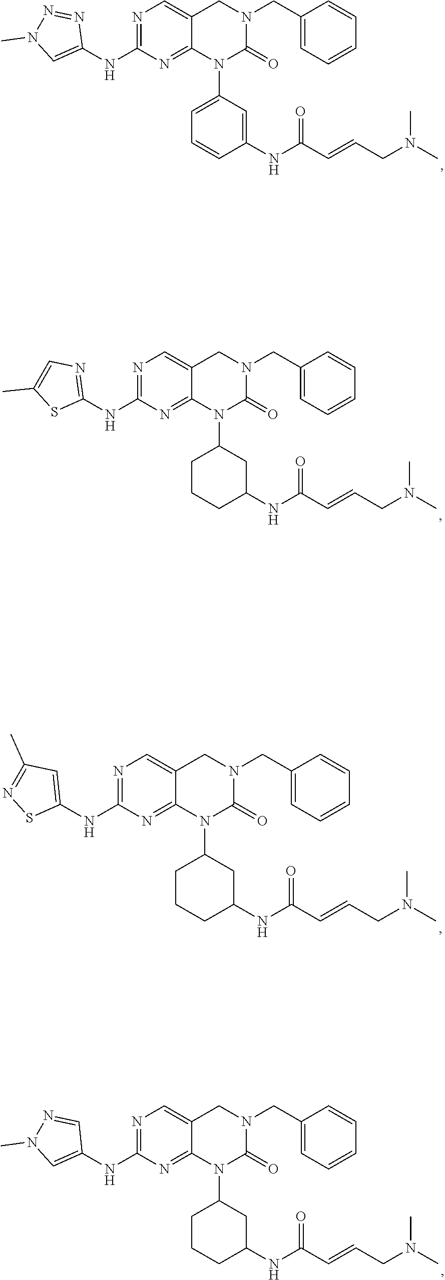

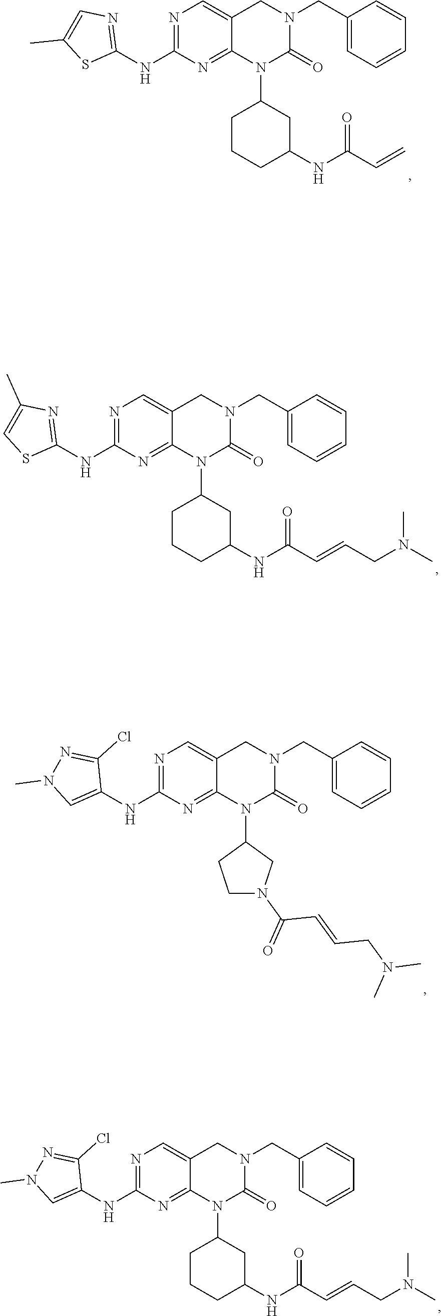

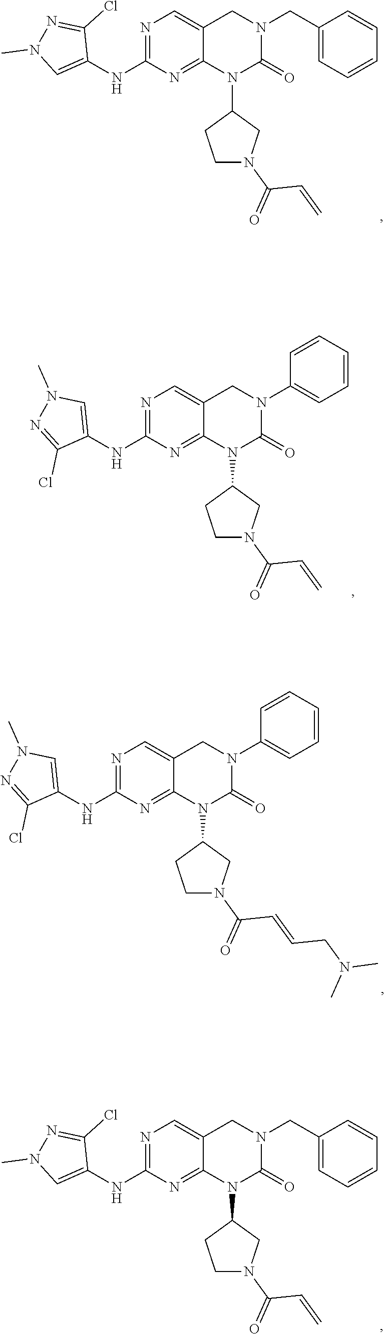

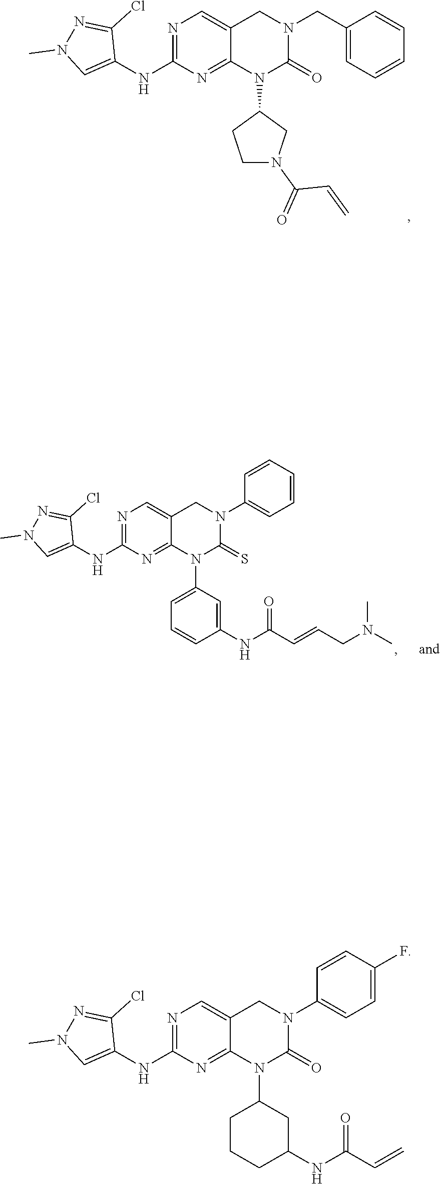

In some embodiments, the compound is selected from:

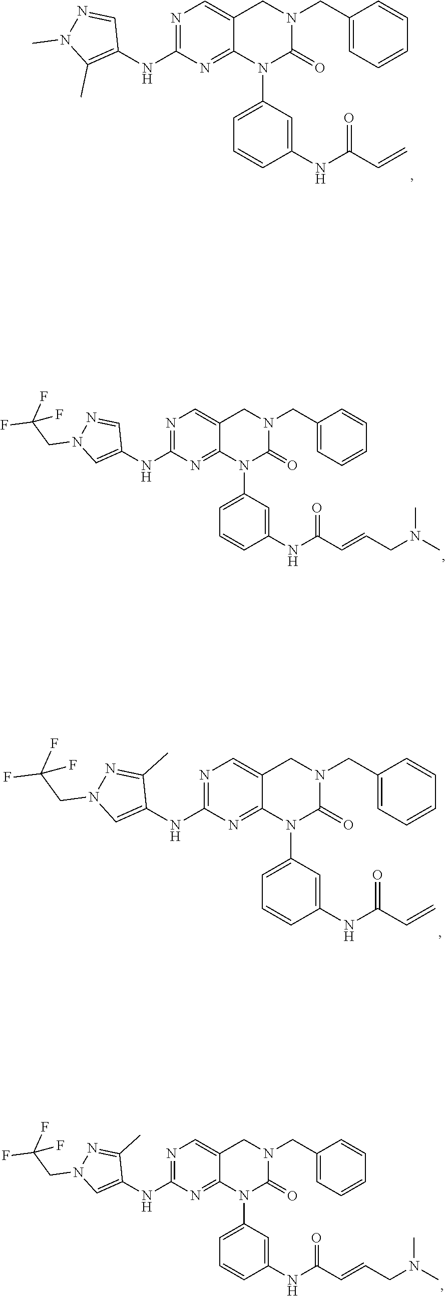 [SMILES: C=CC(=O)Nc1cccc(N2C(=O)N(Cc3ccccc3)Cc3cnc(Nc4cnn(C)c4C)nc32)c1 ; CN(C)C/C=C/C(=O)Nc1cccc(N2C(=O)N(Cc3ccccc3)Cc3cnc(Nc4cnn(CC(F)(F)F)c4)nc32)c1 ; C=CC(=O)Nc1cccc(N2C(=O)N(Cc3ccccc3)Cc3cnc(Nc4cn(CC(F)(F)F)nc4C)nc32)c1 ; Cc1nn(CC(F)(F)F)cc1Nc1ncc2c(n1)N(c1cccc(NC(=O)/C=C/CN(C)C)c1)C(=O)N(Cc1ccccc1)C2]

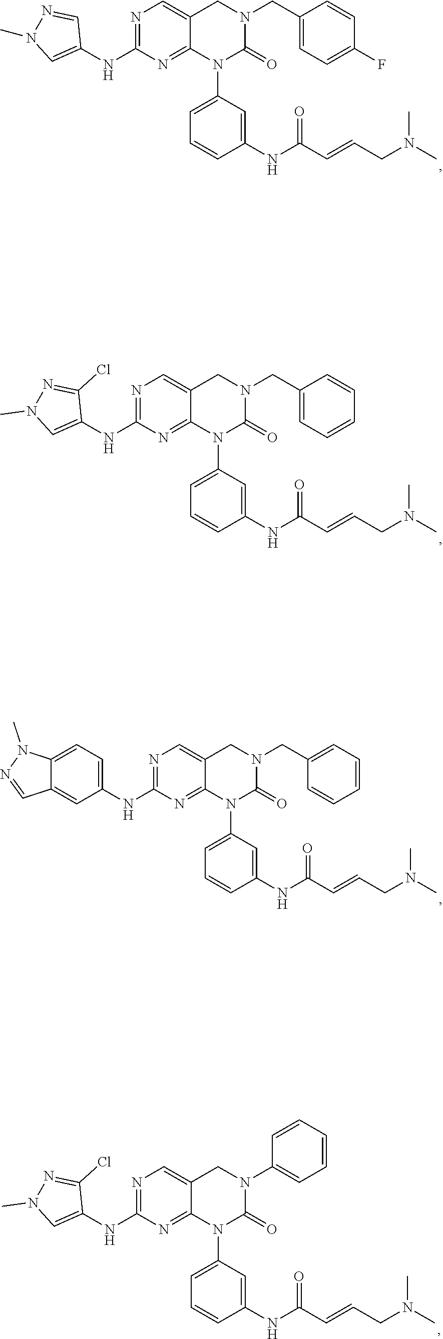

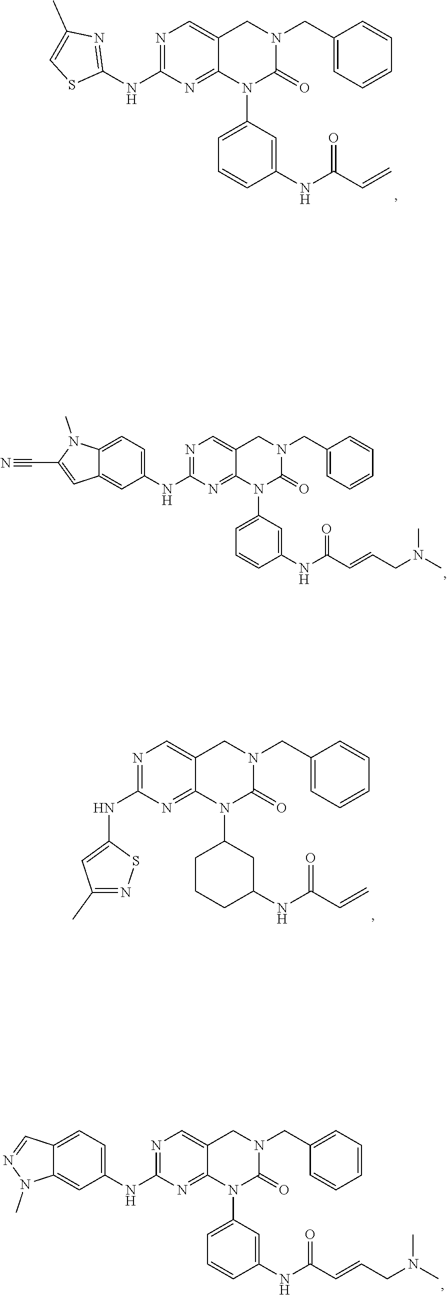

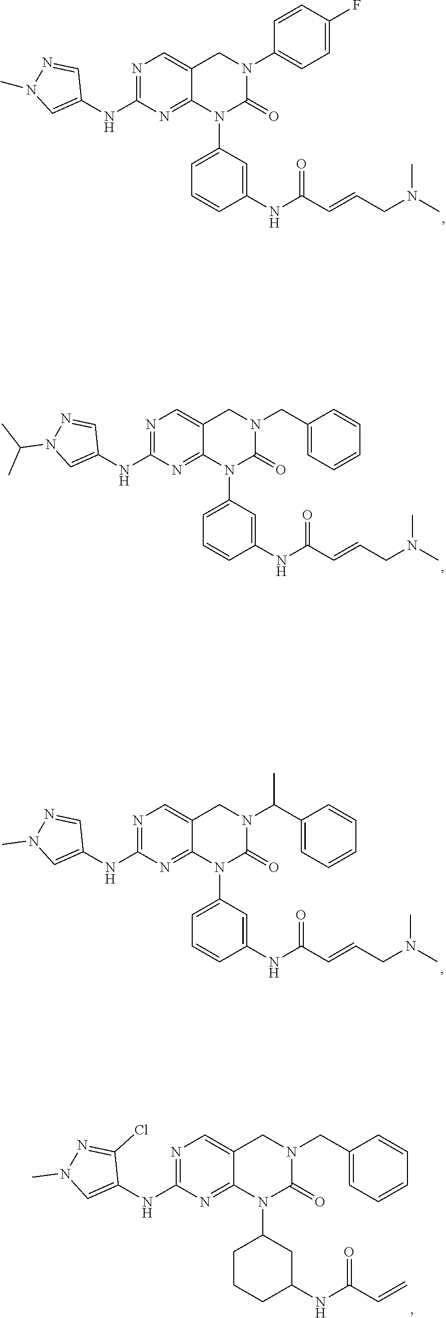

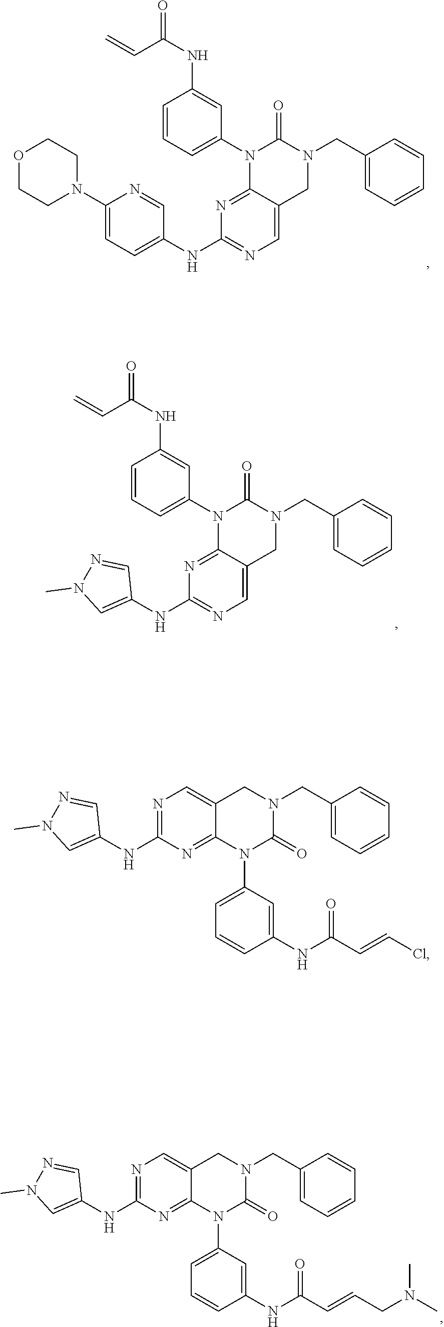

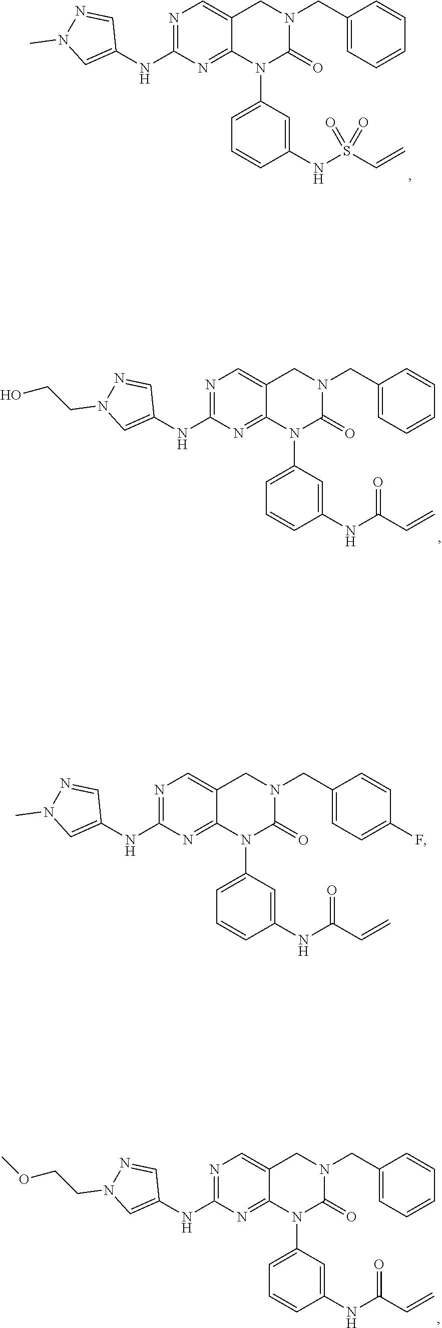 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1]

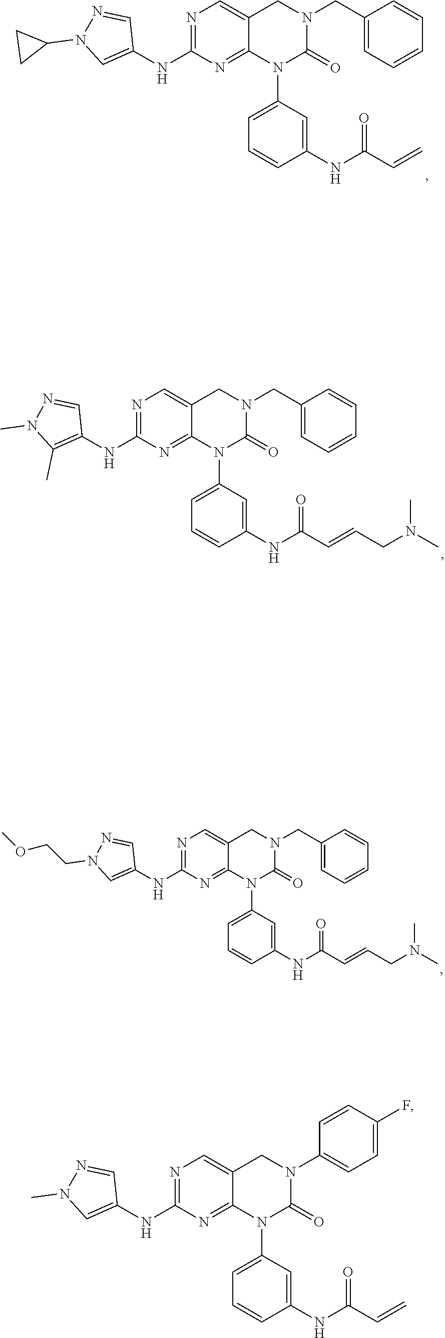

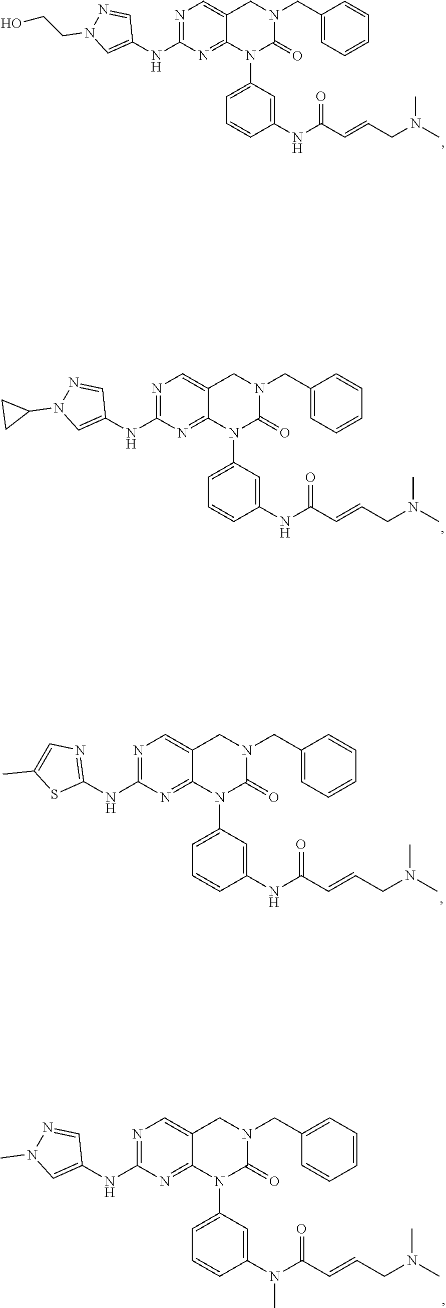

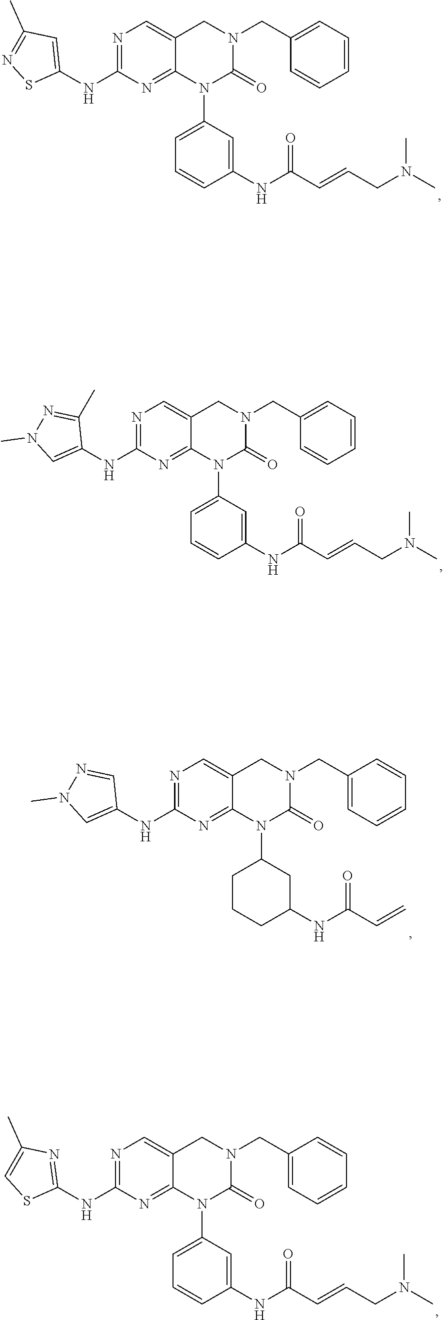

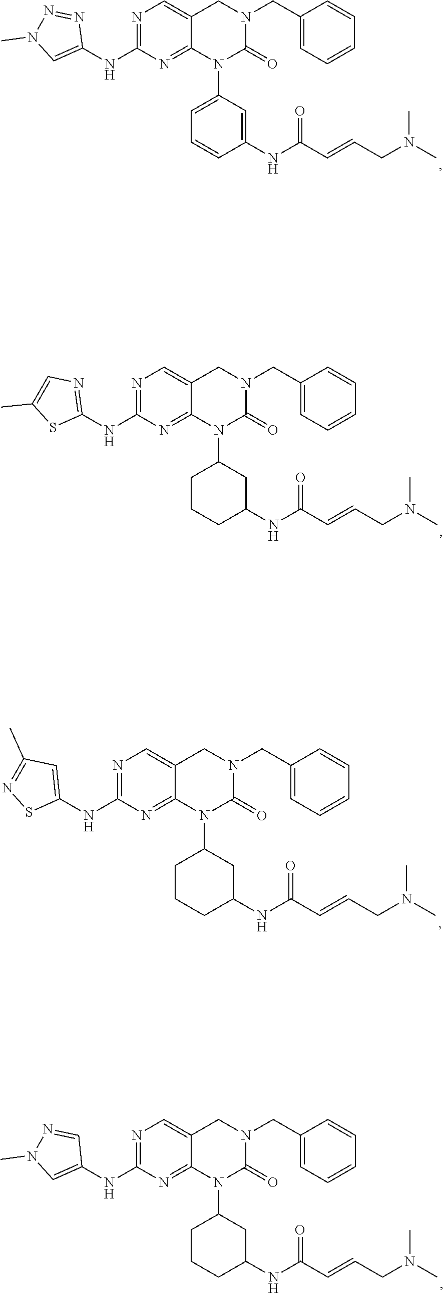

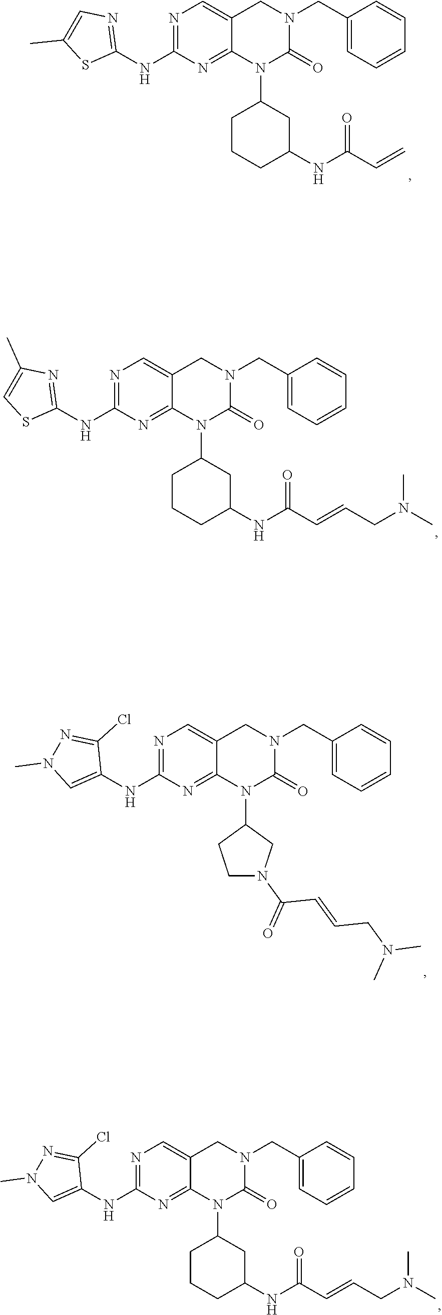

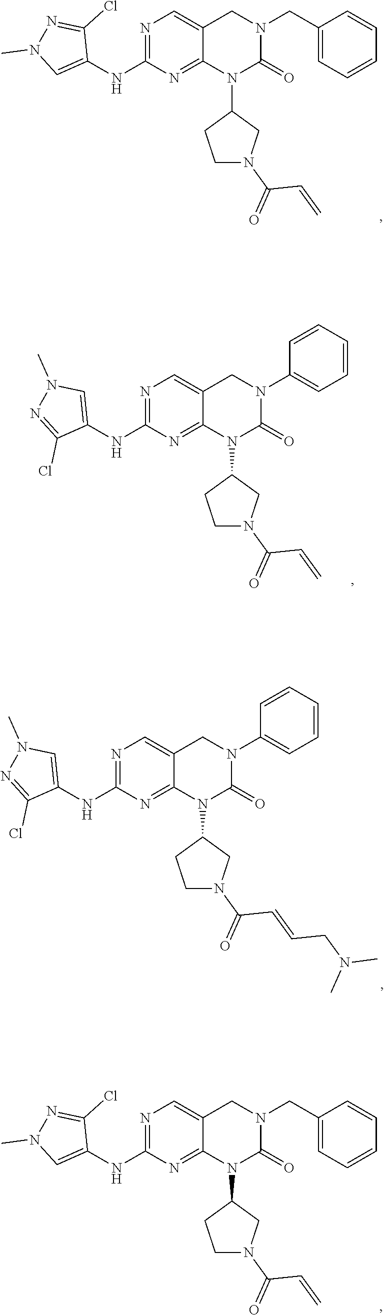

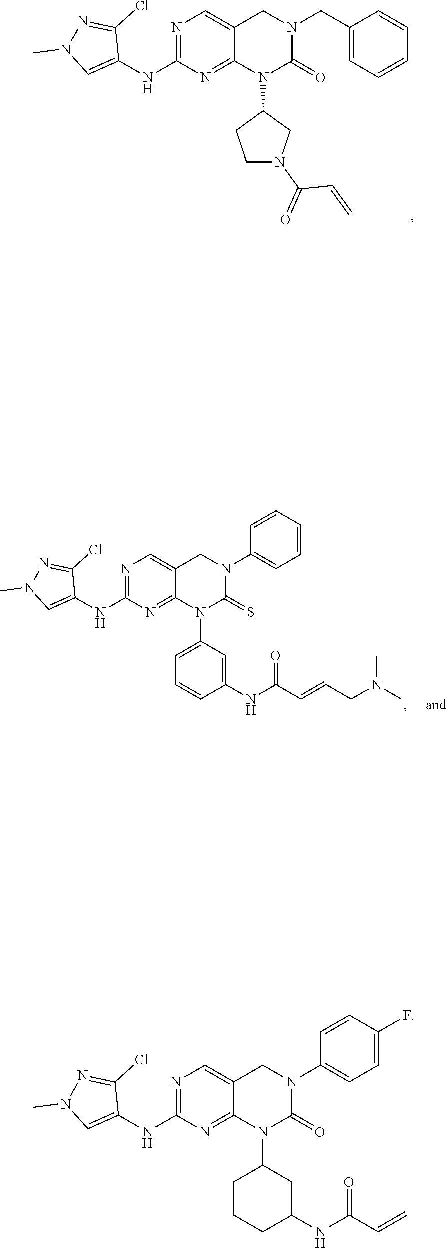

In another aspect, provided herein is a pharmaceutical composition comprising a compound of Formula I, or a pharmaceutically acceptable salt thereof, and a pharmaceutically acceptable carrier.

In another aspect, provided herein is a method of inhibiting an epidermal growth factor receptor (EGFR) family kinase mutant in a subject in need thereof, comprising administering to the subject a therapeutically effective amount of a compound of Formula I, or a pharmaceutically acceptable salt thereof.

In another aspect, provided herein is a method of inhibiting a human epidermal growth factor receptor 2 (HER2) mutant in a subject in need thereof, comprising administering to the subject a therapeutically effective amount of a compound of Formula I, or a pharmaceutically acceptable salt thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775 G776insVVMA, G776del insVC, G776del insLC, G776del insAV, G776del insAVGC, S310F, S310Y, p95, V842I, or P780_Y781insGSP.

In another aspect, provided herein is a method of inhibiting an epidermal growth factor receptor (EGFR) mutant in a subject in need thereof, comprising administering to the subject a therapeutically effective amount of a compound of Formula I, or a pharmaceutically acceptable salt thereof.

In another aspect, provided herein is a method of inhibiting a drug-resistant epidermal growth factor receptor (EGFR) mutant in a subject in need thereof, comprising administering to the subject a therapeutically effective amount of a compound of Formula I, or a pharmaceutically acceptable salt thereof. In some embodiments, the drug-resistant EGFR mutant is del19/T790M EGFR or L858R/T790M EGFR.

In another aspect, provided herein is a method of inhibiting human epidermal growth factor receptor 2 (HER2) in a subject in need thereof, comprising administering to the subject a therapeutically effective amount of a compound of Formula I, or a pharmaceutically acceptable salt thereof, wherein the compound exhibits greater inhibition of a HER2 mutant relative to wild-type EGFR. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780 Y781insGSP.

In another aspect, provided herein is a method of inhibiting epidermal growth factor receptor (EGFR) in a subject in need thereof, comprising administering to the subject a therapeutically effective amount of a compound of Formula I, or a pharmaceutically acceptable salt thereof, wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutant is del19/T790M EGFR or L858R/T790M EGFR.

In another aspect, provided herein is a method of treating a disease or disorder associated with an epidermal growth factor receptor (EGFR) family kinase in a subject in need thereof, comprising administering to the subject a therapeutically effective amount of a compound of Formula I, or a pharmaceutically acceptable salt thereof.

In some embodiments, the disease or disorder in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780 Y781insGSP.

In some embodiments, the disease or disorder in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

In another aspect, provided herein is a method of treating one or more cancer cells in a subject in need thereof, comprising administering to the subject a therapeutically effective amount of a compound of Formula I, or a pharmaceutically acceptable salt thereof.

In another aspect, provided herein is a method of treating cancer in a subject in need thereof, comprising administering to the subject a therapeutically effective amount of a compound of Formula I, or a pharmaceutically acceptable salt thereof.

In some embodiments, the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer. In some embodiments, the cancer is selected from non-small cell lung cancer, prostate cancer, head and neck cancer, breast cancer, colorectal cancer, and glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775 G776insSVMA, A775_G776insVVMA, G776del insVC, G776del insLC, G776del insAV, G776del insAVGC, S310F, S310Y, p95, V842I, or P780_Y781insGSP.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

In another aspect, the present disclosure provides a method of treating an inflammatory disease in a subject in need thereof, comprising administering to the subject a therapeutically effective amount of a compound of Formula I, or a pharmaceutically acceptable salt thereof.

In some embodiments, the inflammatory disease is selected from psoriasis, eczema, and atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR.

The present disclosure discloses a process of preparation of compounds of Formula I, or its stereoisomers, tautomers, pharmaceutically acceptable salts, polymorphs, solvates and hydrates thereof, and to pharmaceutical compositions containing them.

The compounds of the present invention are useful in the treatment, prevention or suppression of diseases and disorders mediated by epidermal growth factor receptor (EGFR).

These and other features, aspects, and advantages of the present disclosure will become better understood with reference to the following description. This statement is provided to introduce a selection of concepts in simplified form. This statement is not intended to identify key features or essential features of the subject matter, nor is it intended to be used to limit the scope of the subject matter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the structural formulae given herein and throughout the present disclosure, the following terms have the indicated meaning, unless specifically stated otherwise.

The term “optionally substituted” as used herein means that the group in question is either unsubstituted or substituted with one or more of the substituents specified. In some embodiments, when the group in question is substituted with more than one substituent, the substituent is the same. In some embodiments, when the group in question is substituted with more than one substituent, the substituent is different.

The term “alkyl” refers to a monoradical branched or unbranched saturated hydrocarbon chain having 1, 2, 3, 4, 5, or 6 carbon atoms. This term is exemplified by groups such as methyl, ethyl, n-propyl, iso-propyl, n-butyl, iso-butyl, t-butyl, n-hexyl, and the like.

The term “cycloalkyl” refers to unless otherwise mentioned, carbocyclic groups of from 3 to 6 carbon atoms having a single cyclic ring or multiple condensed rings or spirocyclic rings or bridged rings. This definition encompasses rings that are saturated or partially unsaturated. Such cycloalkyl groups include, by way of example, single ring structures such as cyclopropyl, cyclobutyl, cyclopentyl, cyclopentenyl, cyclohexyl, cyclohexenyl, and the like.

“Halo” or “Halogen”, alone or in combination with any other term means halogens such as chloro (Cl), fluoro (F), bromo (Br) and iodo (I).

The term “aryl” refers to a radical derived from a hydrocarbon ring system comprising hydrogen, 6 to 30 carbon atoms and at least one aromatic ring. This definition encompasses monocyclic, bicyclic, tricyclic or tetracyclic ring system, as well as fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

The term “phenyl” refers to an aromatic carbocyclic group of 6 carbon atoms having a single ring.

The term “phenyl alkyl” refers to a monoradical branched or unbranched saturated hydrocarbon chain having 1, 2, 3, 4, 5, or 6 carbon atoms substituted with an aromatic carbocyclic group of 6 carbon atoms having a single ring.

The term “heteroaryl” refers to an aromatic cyclic group having 5, or 6 carbon atoms and 1, 2, or 3 heteroatoms selected from oxygen, nitrogen and sulfur within at least one ring. An “X-linked heteroaryl” refers to a heteroaryl connected to the rest of the molecule via an X atom. For example

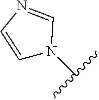 [SMILES: *n1ccnc1]

is an N-linked imidazolyl, while

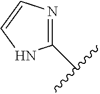 [SMILES: *c1ncc[nH]1]

is a C-linked imidazolyl.

The term “heterocycloalkyl” refers to a saturated, partially unsaturated, or unsaturated group having a single ring or multiple condensed rings or spirocyclic rings, or bridged rings unless otherwise mentioned, having from 2 to 10 carbon atoms and from 1 to 3 hetero atoms, selected from nitrogen, sulfur, phosphorus, and/or oxygen within the ring.

The term “alkenyl” refers to unsaturated aliphatic groups having at least one double bond.

The term “alkynyl” refers to unsaturated aliphatic groups having at least one triple bond.

The term “amino” refers to the —NH₂ radical.

The term “cyano” refers to the —CN radical.

The term “hydroxy” or “hydroxyl” refers to the —OH radical.

The term “heteroalkyl” refers to an alkyl radical as described above where one or more carbon atoms of the alkyl is replaced with an O, N or S atom. Unless stated otherwise specifically in the specification, the heteroalkyl group is optionally substituted as described below. Representative heteroalkyl groups include, but are not limited to —OCH₂CH₂OMe, —OCH₂CH₂OCH₂CH₂NH₂, and —OCH₂CH₂OCH₂CH₂OCH₂CH₂N(Me)₂.

The term “haloalkyl” refers to an alkyl radical as described above where one or more carbon atoms of the alkyl is replaced with a halogen atom. In some embodiments, the haloalkyl group is optionally substituted as described below. Representative haloalkyl groups include, but are not limited to, fluoromethyl, difluoromethyl, trifluoromethyl, difluoroethyl, and trifluoroethyl.

The term “aminoalkyl” refers to an alkyl group substituted with an amino (NH2) group.

The term “alkoxy” refers to the group R—O—, where R is optionally substituted alkyl or optionally substituted cycloalkyl, or optionally substituted alkenyl or optionally substituted alkynyl; or optionally substituted cycloalkenyl, where alkyl, alkenyl, alkynyl, cycloalkyl and cycloalkenyl are as defined herein. Representative examples of alkoxy groups include but are not limited to methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

In some embodiments, the compounds of the present disclosure have the ability to crystallize in more than one form, a characteristic known as polymorphism, and all such polymorphic forms (“polymorphs”) are encompassed within the scope of the disclosure. Polymorphism generally can occur as a response to changes in temperature or pressure or both, and can also result from variations in the crystallization process. Polymorphs can be distinguished by various physical characteristics, and typically the X-ray diffraction patterns, solubility behavior, and melting point of the compound are used to distinguish polymorphs.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, the compounds described herein can exist as isotopic variants. In some embodiments, an isotopic variant of a compound described herein has one or more hydrogen atoms replaced by deuterium.

In some embodiments, the compounds described herein contain one or more chiral centers and/or double bonds and therefore, exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. In some embodiments, the compounds also exist in several tautomeric forms including the enol form, the keto form and mixtures thereof. Accordingly, the chemical structures depicted herein encompass all possible tautomeric forms of the illustrated or identified compounds.

In some embodiments, compounds exist in unsolvated forms as well as solvated forms, including hydrated forms and as N-oxides. In some embodiments, compounds are hydrated, solvated or N-oxides. In some embodiments, certain compounds exist in multiple crystalline or amorphous forms. Also contemplated within the scope of the disclosure are congeners, analogs, hydrolysis products, metabolites and precursor or prodrugs of the compound. In general, unless otherwise indicated, all physical forms are equivalent for the uses contemplated herein and are intended to be within the scope of the present disclosure.

“Pharmaceutically acceptable salt” embraces salts with a pharmaceutically acceptable acid or base. Pharmaceutically acceptable acids include both inorganic acids, for example hydrochloric, sulfuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulfonic, ethanesulfonic, benzenesulfonic orp-toluenesulfonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition comprising a compound of the present disclosure and a pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

“Combined” or “in combination” or “combination” should be understood as a functional coadministration, encompassing scenarios wherein compounds are administered separately, in different formulations, different modes of administration (for example subcutaneous, intravenous or oral) and different times of administration. In some embodiments, the individual compounds of such combinations are administered sequentially in separate pharmaceutical compositions. In some embodiments, the individual compounds of such combinations are administered simultaneously in combined pharmaceutical compositions.

### Compounds

In one aspect, provided herein is a compound of Formula I:

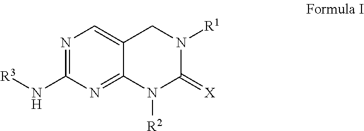

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
   each R⁷ is independently

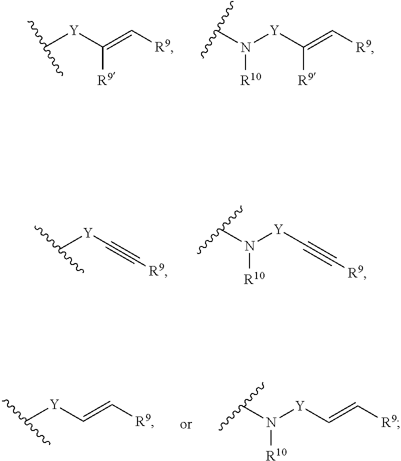

Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹¹ is independently alkyl, cycloalkyl, aryl, or heteroaryl.

In another aspect, provided herein is a compound of Formula I:

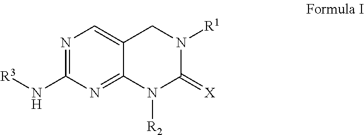

or a pharmaceutically acceptable salt thereof, wherein:
X is O or S;
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with at least one R⁷ and 0, 1, or 2 R⁸;
   each R⁷ is independently

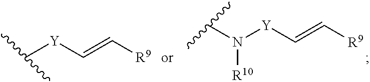

Y is —C(═O)—, —S(═O)—, or —S(═O)₂—;
R⁹ is hydrogen, halo, alkyl, cycloalkyl, or heteroalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is independently hydrogen or alkyl;
   R⁵ is aryl;
   each R^{5′} is halo;
R² is aryl substituted with one R⁷;
   R⁷ is

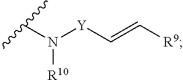

Y is —C(═O)—, or —S(═O)₂—;
R⁹ is hydrogen or heteroalkyl;
R¹⁰ is hydrogen or alkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is aryl substituted with one R;
   R⁷ is

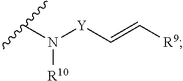

Y is —C(═O)—;
R⁹ is hydrogen or heteroalkyl;
R¹⁰ is hydrogen or alkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is hydrogen;
   R⁵ is aryl;
   each R^{5′} is halo;
R² is cycloalkyl substituted with one R⁷;
   R⁷ is

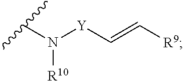

Y is —C(═O)—;
R⁹ is hydrogen or heteroalkyl;
R¹⁰ is hydrogen;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is cycloalkyl substituted with one R⁷;
   R⁷ is

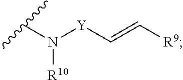

Y is —C(═O)—;
R⁹ is hydrogen;
R¹⁰ is hydrogen;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 1;
   each R⁴ is hydrogen;
   R⁵ is aryl;
   each R^{5′} is halo;
R² is heterocycloalkyl substituted with one R⁷;
   R⁷ is

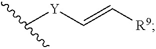

Y is —C(═O)—;
R⁹ is hydrogen or heteroalkyl;
R³ is heteroaryl substituted with 1 or 2 R¹²; and
   each R¹² is independently alkyl or halo.

Some embodiments provided herein describe a compound of Formula I or a pharmaceutically acceptable salt thereof, wherein:
X is O;
R¹ is —(C(R⁴)₂)ₙR⁵, wherein R⁵ is substituted with 0 or 1 R^{5′};
   n is 0;
   R⁵ is aryl;
   each R^{5′} is halo;
R² is heterocycloalkyl substituted with one R⁷;
   R⁷ is

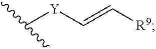

Y is —C(═O)—;
R⁹ is hydrogen;
R³ is heteroaryl substituted with 1 or 2 R¹²; and
   each R¹² is independently alkyl or halo. Some embodiments provided herein describe a compound of Formula I-A

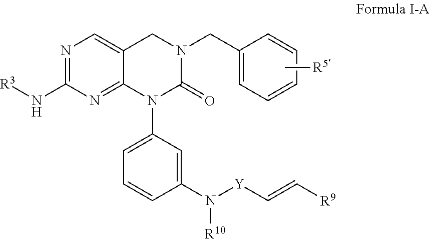

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹¹ is independently alkyl, cycloalkyl, aryl, or heteroaryl.

Some embodiments provided herein describe a compound of Formula I-B

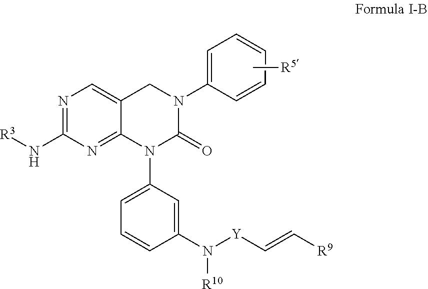

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-C

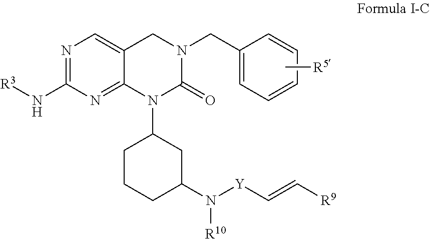

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
         each R¹¹ is independently alkyl, cycloalkyl, aryl, or heteroaryl.

Some embodiments provided herein describe a compound of Formula I-D

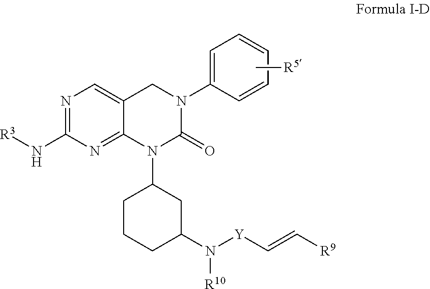

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
      R¹⁰ is hydrogen or alkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-E

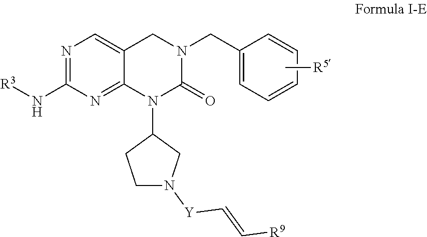

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹⁵ is independently alkyl, cycloalkyl, aryl, or heteroaryl.

Some embodiments provided herein describe a compound of Formula I-F

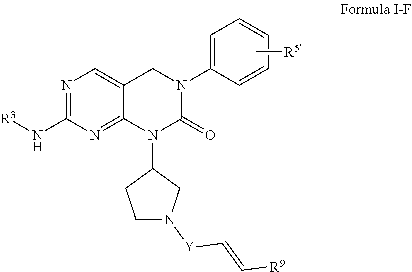

or a pharmaceutically acceptable salt thereof, wherein:
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
      Y is —C(═O)—, —S(═O)—, or —S(═O)₂—;
      R⁹ is hydrogen, halo, alkyl, cycloalkyl, or heteroalkyl;
R³ is heteroaryl substituted with 0, 1, 2, or 3 R¹²;
   each R¹² is independently alkyl, heteroalkyl, haloalkyl, halo, cyano, alkoxy, heterocycloalkyl, —N(R¹³)₂, or cycloalkyl, wherein the heterocycloalkyl or cycloalkyl are each independently substituted with 0, 1, or 2 R¹⁴;
      each R¹³ is independently alkyl, cycloalkyl, aryl, or heteroaryl
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
         each R¹¹ is independently alkyl, cycloalkyl, aryl, or heteroaryl.

Some embodiments provided herein describe a compound of Formula I-G

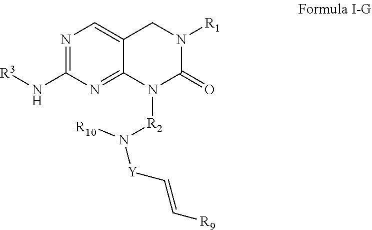

or a pharmaceutically acceptable salt thereof, wherein:
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with 0, 1, or 2 R⁸;
   Y is —C(═O)—, —S(═O)—, or —S(═O)₂—;
   R⁹ is hydrogen, halo, alkyl, cycloalkyl, or heteroalkyl;
   R¹⁰ is hydrogen, alkyl, haloalkyl, or cycloalkyl;
   each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
      each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
      each R¹³ is independently alkyl, cycloalkyl, aryl, or heteroaryl;
      each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
each R¹¹ is independently alkyl, cycloalkyl, aryl, or heteroaryl.

Some embodiments provided herein describe a compound of Formula I-H

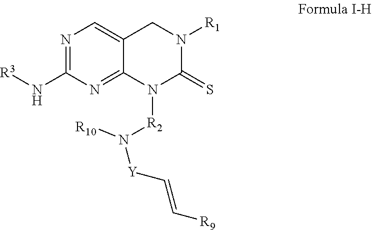

or a pharmaceutically acceptable salt thereof, wherein:
R¹ is —(C(R⁴)₂)ₙR⁵, wherein R⁵ is substituted with 0, 1, or 2 R^{5′};
   n is 0, 1, 2, or 3;
   each R⁴ is independently hydrogen, alkyl, halo, haloalkyl, hydroxy, alkoxy, or heteroalkyl;
   R⁵ is aryl or C-linked heteroaryl;
   each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
      each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with 0, 1, or 2 R⁸;
   Y is —C(═O)—, —S(═O)—, or —S(═O)₂—;
   R⁹ is hydrogen, halo, alkyl, cycloalkyl, or heteroalkyl;
   R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
   each R¹¹ is independently alkyl, cycloalkyl, aryl, or heteroaryl;
R³ is heteroaryl substituted with 0, 1, 2, or 3 R¹²;
   each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
   each R¹³ is independently alkyl, cycloalkyl, aryl, or heteroaryl;
   each R¹⁴ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

For any and all of the embodiments, substituents are selected from among a subset of the listed alternatives. For example, in some embodiments, n is 0, 1, 2, or 3. In some embodiments, n is 0, 1, or 2. In some embodiments, n is 0, 1, or 3. In some embodiments, n is 0, 2, or 3. In some embodiments, n is 1, 2, or 3. In some embodiments, n is 0 or 1. In some embodiments, n is 1 or 2. In some embodiments, n is 2 or 3. In some embodiments, n is 0 or 2. In some embodiments, n is 0 or 3. In some embodiments, n is 1 or 3. In some embodiments, n is 0. In some embodiments, n is 1. In some embodiments, n is 2. In some embodiments, n is 3. In some embodiments, R⁵ is phenyl, naphthyl, anthracenyl, phenanthrenyl, chrysenyl, pyrenyl, C-linked pyridyl, C-linked pyrimidinyl, C-linked pyrazolyl, or C-linked imidazolyl. In some embodiments, R⁵ is phenyl, naphthyl, anthracenyl, phenanthrenyl, C-linked pyridyl, C-linked pyrimidinyl, C-linked pyrazolyl, or C-linked imidazolyl. In some embodiments, R⁵ is phenyl. In some embodiments, R⁵ is naphthyl. In some embodiments, R⁵ is anthracenyl. In some embodiments, R⁵ is phenanthrenyl. In some embodiments, R⁵ is chrysenyl. In some embodiments, R⁵ is pyrenyl. In some embodiments, R⁵ is C-linked pyridyl. In some embodiments, R⁵ is C-linked pyrimidinyl. In some embodiments, R⁵ is C-linked pyrazolyl. In some embodiments, R⁵ is C-linked imidazolyl.

In some embodiments, R⁵ is unsubstituted. In some embodiments, R⁵ is substituted with 0, 1, or 2 R^{5′}. In some embodiments, R⁵ is substituted with 0 or 1 R^{5′}. In some embodiments, R⁵ is substituted with 0 or 2 R^{5′}. In some embodiments, R⁵ is substituted with 1 or 2 R^{5′}. In some embodiments, R⁵ is substituted with 1 R^{5′}. In some embodiments, R⁵ is substituted with 2 R^{5′}.

In some embodiments, each R⁴ is independently hydrogen, alkyl, halo, haloalkyl, hydroxy, alkoxy, or heteroalkyl. In some embodiments, each R⁴ is independently hydrogen, alkyl, halo, haloalkyl, or alkoxy. In some embodiments, each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy. In some embodiments, each R⁴ is independently hydrogen, methyl, fluoro, trifluoromethyl, methoxy, or trifluoromethoxy. In some embodiments, each R⁴ is hydrogen. In some embodiments, each R⁴ is independently alkyl. In some embodiments, each R⁴ is independently halo. In some embodiments, each R⁴ is independently haloalkyl. In some embodiments, each R⁴ is hydroxy. In some embodiments, each R⁴ is independently alkoxy. In some embodiments, each R⁴ is independently heteroalkyl. In some embodiments, each R⁴ is methyl. In some embodiments, each R⁴ is ethyl. In some embodiments, each R⁴ is n-propyl. In some embodiments, each R⁴ is iso-propyl. In some embodiments, each R⁴ is n-butyl. In some embodiments, each R⁴ is iso-butyl. In some embodiments, each R⁴ is sec-butyl. In some embodiments, each R⁴ is tert-butyl. In some embodiments, each R⁴ is fluoro. In some embodiments, each R⁴ is chloro. In some embodiments, each R⁴ is trifluoromethyl. In some embodiments, each R⁴ is trifluoroethyl. In some embodiments, each R⁴ is pentafluoroethyl. In some embodiments, each R⁴ is methoxy. In some embodiments, each R⁴ is ethoxy. In some embodiments, each R⁴ is trifluoromethoxy.

In some embodiments, each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy. In some embodiments, each R^{5′} is independently aryl, heteroaryl, alkyl, heterocycloalkyl, halo, cyano, hydroxy, —N(R⁶)₂, or alkoxy. In some embodiments, each R^{5′} is independently aryl. In some embodiments, each R^{5′} is independently heteroaryl. In some embodiments, each R^{5′} is independently alkyl. In some embodiments, each R^{5′} is independently cycloalkyl. In some embodiments, each R^{5′} is independently heterocycloalkyl. In some embodiments, each R^{5′} is independently halo. In some embodiments, each R^{5′} is independently heteroalkyl. In some embodiments, each R^{5′} is independently haloalkyl. In some embodiments, each R^{5′} is cyano. In some embodiments, each R^{5′} is hydroxy. In some embodiments, each R^{5′} is amino. In some embodiments, each R^{5′} is independently —N(R⁶)₂. In some embodiments, each R^{5′} is independently —S(═O)₂alkyl. In some embodiments, each R^{5′} is independently —S(═O)₂aryl. In some embodiments, each R^{5′} is independently-S(═O)₂heteroaryl. In some embodiments, each R^{5′} is independently alkoxy. In some embodiments, each R^{5′} is independently phenyl, naphthyl, anthracenyl, phenanthrenyl, chrysenyl, pyrenyl, pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, naphthyridinyl, methyl, ethyl, n-propyl, iso-propyl, n-butyl, iso-butyl, sec-butyl, tert-butyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, hydroxy, —N(R⁶)₂, methoxy, ethoxy, or trifluoromethoxy. In some embodiments, each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy. In some embodiments, each R^{5′} is independently phenyl, imidazolyl, pyridinyl, methyl, tert-butyl, pyrrolidinyl, morpholinyl, fluoro, cyano, hydroxy, —N(R⁶)₂, or methoxy. In some embodiments, each R^{5′} is phenyl. In some embodiments, each R^{5′} is naphthyl. In some embodiments, each R^{5′} is anthracenyl. In some embodiments, each R^{5′} is phenanthrenyl. In some embodiments, each R^{5′} is chrysenyl. In some embodiments, each R^{5′} is pyrenyl. In some embodiments, each R^{5′} is pyrrolyl. In some embodiments, each R^{5′} is imidazolyl. In some embodiments, each R^{5′} is pyrazolyl. In some embodiments, each R^{5′} is triazolyl. In some embodiments, each R^{5′} is tetrazolyl. In some embodiments, each R^{5′} is indolyl. In some embodiments, each R^{5′} is indazolyl. In some embodiments, each R^{5′} is benzimidazolyl. In some embodiments, each R^{5′} is azaindolyl. In some embodiments, each R^{5′} is thiazolyl. In some embodiments, each R^{5′} is isothiazolyl. In some embodiments, each R^{5′} is oxazolyl. In some embodiments, each R^{5′} is isoxazolyl. In some embodiments, each R^{5′} is pyridinyl. In some embodiments, each R^{5′} is pyrimidinyl. In some embodiments, each R^{5′} is pyridazinyl. In some embodiments, each R^{5′} is pyrazinyl. In some embodiments, each R^{5′} is triazinyl. In some embodiments, each R^{5′} is quinolinyl. In some embodiments, each R^{5′} is isoquinolinyl. In some embodiments, each R^{5′} is quinoxalinyl. In some embodiments, each R^{5′} is quinazolinyl. In some embodiments, each R^{5′} is cinnolinyl. In some embodiments, each R^{5′} is naphthyridinyl. In some embodiments, each R^{5′} is methyl. In some embodiments, each R^{5′} is ethyl. In some embodiments, each R^{5′} is n-propyl. In some embodiments, each R^{5′} is iso-propyl. In some embodiments, each R^{5′} is n-butyl. In some embodiments, each R^{5′} is iso-butyl. In some embodiments, each R^{5′} is sec-butyl. In some embodiments, each R^{5′} is tert-butyl. In some embodiments, each R^{5′} is azetidinyl. In some embodiments, each R^{5′} is oxetanyl. In some embodiments, each R^{5′} is pyrrolidinyl. In some embodiments, each R^{5′} is imidazolidinyl. In some embodiments, each R^{5′} is tetrahydrofuranyl. In some embodiments, each R^{5′} is piperidinyl. In some embodiments, each R^{5′} is piperazinyl. In some embodiments, each R^{5′} is tetrahydropyranyl. In some embodiments, each R^{5′} is morpholinyl. In some embodiments, each R^{5′} is fluoro. In some embodiments, each R^{5′} is chloro. In some embodiments, each R^{5′} is methoxy. In some embodiments, each R^{5′} is ethoxy. In some embodiments, each R^{5′} is trifluoromethoxy.

In some embodiments, each R⁶ is independently alkyl, cycloalkyl, aryl, or heteroaryl. In some embodiments, each R⁶ is independently alkyl or aryl. In some embodiments, each R⁶ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R⁶ is independently methyl, ethyl, iso-propyl, tert-butyl, phenyl, or naphthyl. In some embodiments, each R⁶ is independently methyl or phenyl. In some embodiments, each R⁶ is methyl. In some embodiments, each R⁶ is ethyl. In some embodiments, each R⁶ is n-propyl. In some embodiments, each R⁶ is iso-propyl. In some embodiments, each R⁶ is n-butyl. In some embodiments, each R⁶ is iso-butyl. In some embodiments, each R⁶ is sec-butyl. In some embodiments, each R⁶ is tert-butyl. In some embodiments, each R⁶ is phenyl. In some embodiments, each R⁶ is naphthyl. In some embodiments, each R⁶ is anthracenyl. In some embodiments, each R⁶ is phenanthrenyl. In some embodiments, each R⁶ is chrysenyl. In some embodiments, each R⁶ is pyrenyl.

In some embodiments, X is S. In some embodiments, X is O.

In some embodiments, R² is aryl, heteroaryl, cycloalkyl, or heterocycloalkyl. In some embodiments, R² is aryl. In some embodiments, R² is heteroaryl. In some embodiments, R² is cycloalkyl. In some embodiments, R² is heterocycloalkyl. In some embodiments, R² is monocyclic. In some embodiments, R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl. In some embodiments, R² is phenyl, cyclohexyl, or pyrrolyl. In some embodiments, R² is phenyl. In some embodiments, R² is cyclopropyl. In some embodiments, R² is cyclobutyl. In some embodiments, R² is cyclopentyl. In some embodiments, R² is cyclohexyl. In some embodiments, R² is pyrrolyl. In some embodiments, R² is imidazolyl. In some embodiments, R² is pyrazolyl. In some embodiments, R² is triazolyl. In some embodiments, R² is tetrazolyl. In some embodiments, R² is thiazolyl. In some embodiments, R² is isothiazolyl. In some embodiments, R² is oxazolyl. In some embodiments, R² is isoxazolyl. In some embodiments, R² is pyridinyl. In some embodiments, R² is pyrimidinyl. In some embodiments, R² is pyridazinyl. In some embodiments, R² is pyrazinyl. In some embodiments, R² is triazinyl.

In some embodiments, R⁷ is

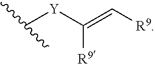

In some embodiments, R⁷ is

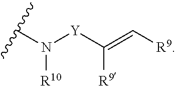

In some embodiments, R⁷ is

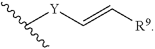

In some embodiments, R⁷ is

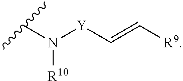

In some embodiments, R⁷ is

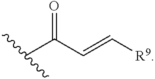

In some embodiments, R⁷ is

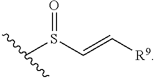

In some embodiments, R⁷ is

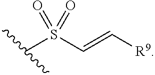

In some embodiments, R⁷ is

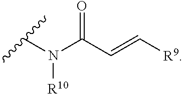

In some embodiments, R⁷ is

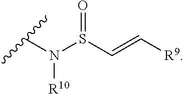

In some embodiments, R⁷ is

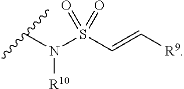

In some embodiments, R is

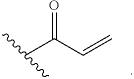 [SMILES: *C(=O)C=C]

In some embodiments, R⁷ is

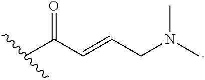 [SMILES: *C(=O)/C=C/CN(C)C]

In some embodiments, R⁷ is

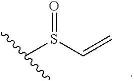 [SMILES: *S(=O)C=C]

In some embodiments, R⁷ is

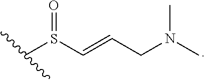 [SMILES: *S(=O)/C=C/CN(C)C]

In some embodiments, R⁷ is

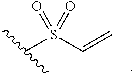 [SMILES: *S(=O)(=O)C=C]

In some embodiments, R⁷ is

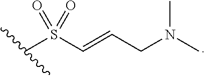 [SMILES: *S(=O)(=O)/C=C/CN(C)C]

In some embodiments, R⁷ is

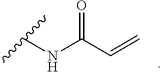 [SMILES: *NC(=O)C=C]

In some embodiments, R⁷ is

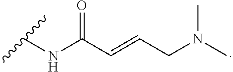 [SMILES: *NC(=O)/C=C/CN(C)C]

In some embodiments, R⁷ is

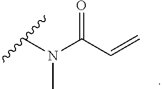 [SMILES: *N(C)C(=O)C=C]

In some embodiments, R⁷ is

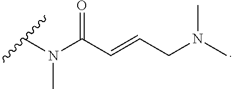 [SMILES: *N(C)C(=O)/C=C/CN(C)C]

In some embodiments, R⁷ is

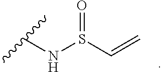 [SMILES: *NS(=O)C=C]

In some embodiments, R⁷ is

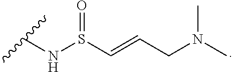 [SMILES: *NS(=O)/C=C/CN(C)C]

In some embodiments, R⁷ is

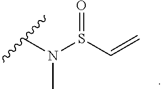 [SMILES: *N(C)S(=O)C=C]

In some embodiments, R⁷ is

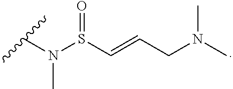 [SMILES: *N(C)S(=O)/C=C/CN(C)C]

In some embodiments, R⁷ is

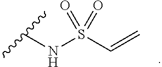 [SMILES: *NS(=O)(=O)C=C]

In some embodiments, R⁷ is

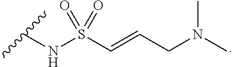 [SMILES: *NS(=O)(=O)/C=C/CN(C)C]

In some embodiments, R⁷ is

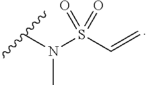 [SMILES: *N(C)S(=O)(=O)C=C]

In some embodiments, R⁷ is

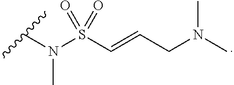 [SMILES: *N(C)S(=O)(=O)/C=C/CN(C)C]

In some embodiments, Y is —C(═O)—. In some embodiments, Y is —S(═O)—. In some embodiments, Y is —S(═O)₂—.

In some embodiments, R⁹ and R^{9′} are independently hydrogen, halo, alkyl, heteroalkyl, haloalkyl, or (alkyl)heterocycloalkyl. In some embodiments, R⁹ is hydrogen, halo, alkyl, cycloalkyl, or heteroalkyl. In some embodiments, R⁹ is hydrogen, halo, or heteroalkyl. In some embodiments, R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl. In some embodiments, R⁹ is hydrogen, fluoro, chloro, hydroxyethyl, or methoxyethyl. In some embodiments, R⁹ is hydrogen. In some embodiments, R⁹ is fluoro. In some embodiments, R⁹ is chloro. In some embodiments, R⁹ is hydroxyethyl. In some embodiments, R⁹ is methoxyethyl. In some embodiments, R⁹ is methyl. In some embodiments, R⁹ is methoxymethyl. In some embodiments, R⁹ is dimethylaminomethyl. In some embodiments, R⁹ is 1-piperidinylmethyl. In some embodiments, R⁹ is 1-morpholinomethyl. In some embodiments, R⁹ is fluoromethyl. In some embodiments, R^{9′} is hydrogen. In some embodiments, R^{9′} is fluoro. In some embodiments, R^{9′} is chloro. In some embodiments, R^{9′} is hydroxyethyl. In some embodiments, R^{9′} is methoxyethyl. In some embodiments, R^{9′} is methyl. In some embodiments, R^{9′} is methoxymethyl. In some embodiments, R^{9′} is dimethylaminomethyl. In some embodiments, R^{9′} is 1-piperidinylmethyl. In some embodiments, R^{9′} is 1-morpholinomethyl. In some embodiments, R^{9′} is fluoromethyl.

In some embodiments, R¹⁰ is hydrogen or alkyl. In some embodiments, R¹⁰ is hydrogen, methyl, ethyl, n-propyl, iso-propyl, n-butyl, iso-butyl, sec-butyl, or tert-butyl. In some embodiments, R¹⁰ is hydrogen. In some embodiments, R¹⁰ is methyl. In some embodiments, R¹⁰ is ethyl. In some embodiments, R¹⁰ is n-propyl. In some embodiments, R¹⁰ is iso-propyl. In some embodiments, R¹⁰ is n-butyl. In some embodiments, R¹⁰ is iso-butyl. In some embodiments, R¹⁰ is sec-butyl. In some embodiments, R¹⁰ is tert-butyl.

In some embodiments, R² is unsubstituted. In some embodiments, R² is substituted with 1 or 2 R⁸. In some embodiments, R² is substituted with 1 R⁸. In some embodiments, R² is substituted with 2 R⁸.

In some embodiments, each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy. In some embodiments, each R⁸ is independently methyl, ethyl, iso-propyl, tert-butyl, fluoro, chloro, —N(R¹¹)₂, hydroxyethyl, methoxyethyl, or cyano. In some embodiments, each R⁸ is methyl. In some embodiments, each R⁸ is ethyl. In some embodiments, each R⁸ is n-propyl. In some embodiments, each R⁸ is iso-propyl. In some embodiments, each R⁸ is n-butyl. In some embodiments, each R⁸ is iso-butyl. In some embodiments, each R⁸ is sec-butyl. In some embodiments, each R⁸ is tert-butyl. In some embodiments, each R⁸ is fluoro. In some embodiments, each R⁸ is chloro. In some embodiments, each R⁸ is independently —N(R¹¹)₂. In some embodiments, each R⁸ is hydroxyethyl. In some embodiments, each R⁸ is methoxyethyl. In some embodiments, each R⁸ is cyano.

In some embodiments, each R¹¹ is independently alkyl, cycloalkyl, aryl, or heteroaryl. In some embodiments, each R¹¹ is independently alkyl or aryl. In some embodiments, each R¹¹ is independently alkyl. In some embodiments, each R¹¹ is independently cycloalkyl. In some embodiments, each R¹¹ is independently aryl. In some embodiments, each R¹¹ is independently heteroaryl. In some embodiments, each R¹¹ is independently methyl, ethyl, n-propyl, iso-propyl, n-butyl, iso-butyl, sec-butyl, tert-butyl, phenyl, naphthyl, anthracenyl, phenanthrenyl, chrysenyl, or pyrenyl. In some embodiments, each R¹¹ is independently methyl, ethyl, iso-propyl, tert-butyl, phenyl, or naphthyl. In some embodiments, each R¹¹ is independently methyl or phenyl. In some embodiments, each R¹¹ is methyl. In some embodiments, each R¹¹ is ethyl. In some embodiments, each R¹¹ is n-propyl. In some embodiments, each R¹¹ is iso-propyl. In some embodiments, each R¹¹ is n-butyl. In some embodiments, each R¹¹ is iso-butyl. In some embodiments, each R¹¹ is sec-butyl. In some embodiments, each R¹¹ is tert-butyl. In some embodiments, each R¹¹ is phenyl. In some embodiments, each R¹¹ is naphthyl. In some embodiments, each R¹¹ is anthracenyl. In some embodiments, each R¹¹ is phenanthrenyl. In some embodiments, each R¹¹ is chrysenyl. In some embodiments, each R¹¹ is pyrenyl.

In some embodiments, R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl. In some embodiments, R³ is pyrazolyl, triazolyl, indolyl, indazolyl, thiazolyl, isothiazolyl, or pyridinyl. In some embodiments, R³ is pyrrolyl. In some embodiments, R³ is imidazolyl. In some embodiments, R³ is pyrazolyl. In some embodiments, R³ is triazolyl. In some embodiments, R³ is tetrazolyl. In some embodiments, R³ is indolyl. In some embodiments, R³ is indazolyl. In some embodiments, R³ is benzimidazolyl. In some embodiments, R³ is azaindolyl. In some embodiments, R³ is thiazolyl. In some embodiments, R³ is isothiazolyl. In some embodiments, R³ is oxazolyl. In some embodiments, R³ is isoxazolyl. In some embodiments, R³ is pyridinyl. In some embodiments, R³ is pyrimidinyl. In some embodiments, R³ is pyridazinyl. In some embodiments, R³ is pyrazinyl. In some embodiments, R³ is triazinyl. In some embodiments, R³ is quinolinyl. In some embodiments, R³ is isoquinolinyl. In some embodiments, R³ is quinoxalinyl. In some embodiments, R³ is quinazolinyl. In some embodiments, R³ is cinnolinyl. In some embodiments, R³ is naphthyridinyl.

In some embodiments, R³ is unsubstituted. In some embodiments, R³ is substituted with at least 1 R¹². In some embodiments, R³ is substituted with at least 2 R¹². In some embodiments, R³ is substituted with 1 R¹². In some embodiments, R³ is substituted with 2 R¹². In some embodiments, R³ is substituted with 3 R¹².

In some embodiments, R³ is

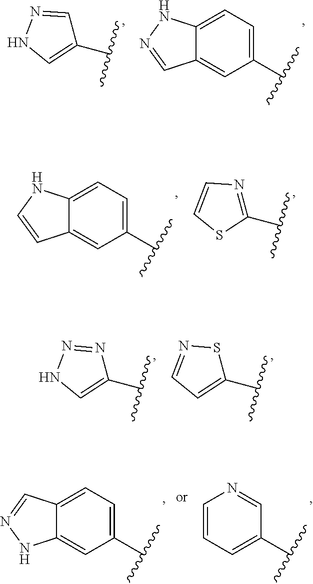

wherein R³ is substituted with 0 to 3 R¹². In some embodiments, R³ is

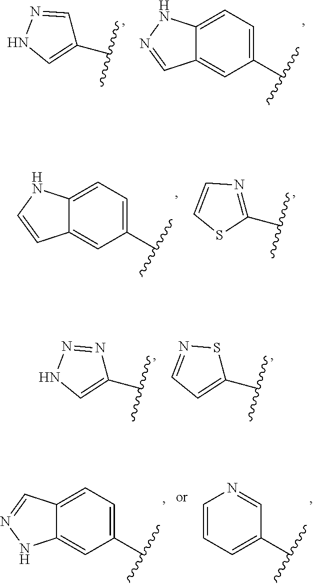

wherein R³ is substituted with 1 or 2 R¹².

In some embodiments, R³ is selected from:

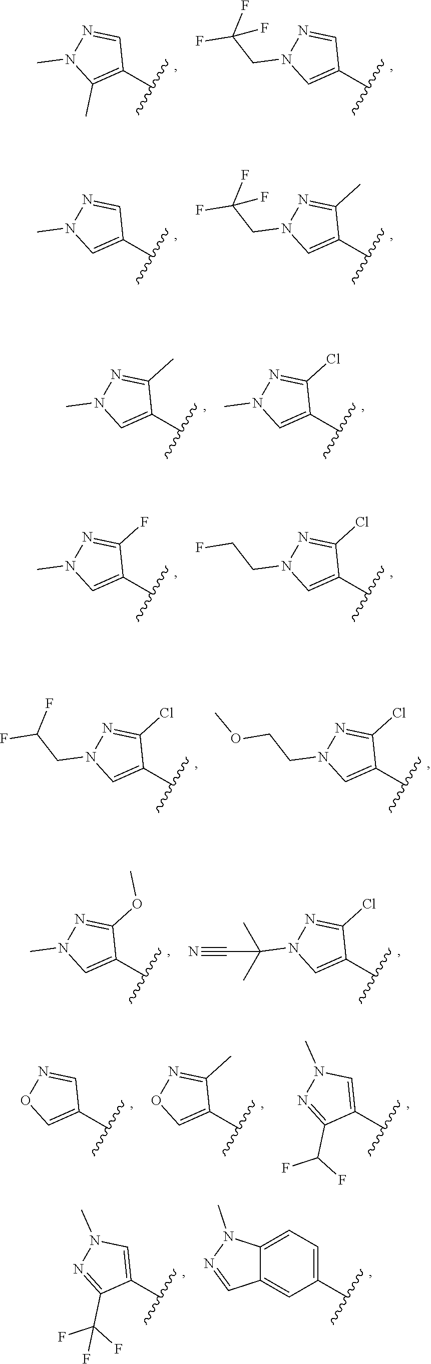

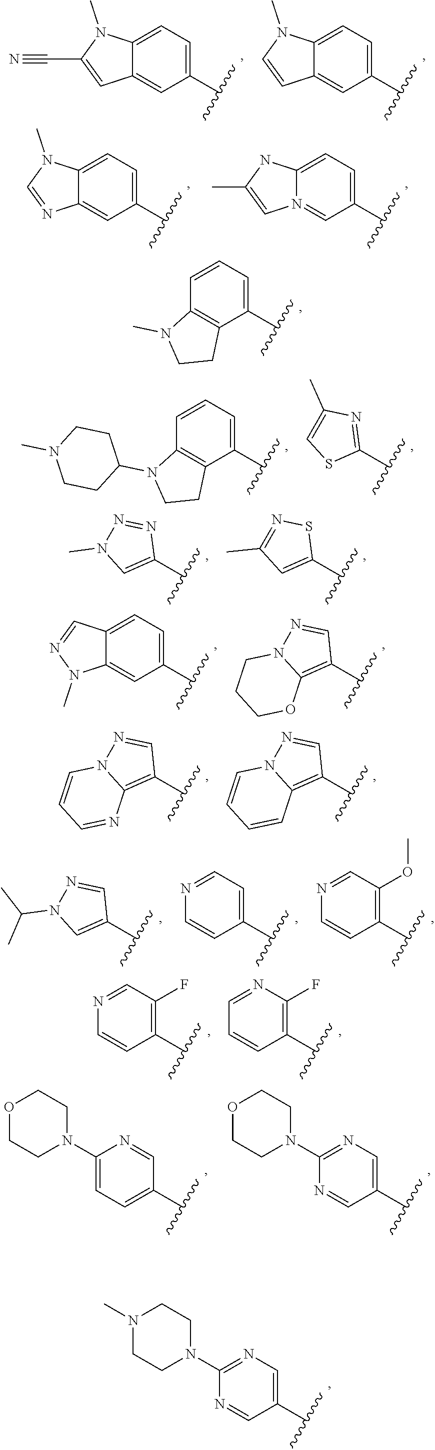

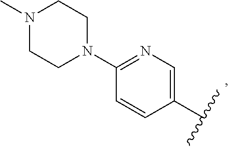 [SMILES: *c1ccc(N2CCN(C)CC2)nc1 ; ","]

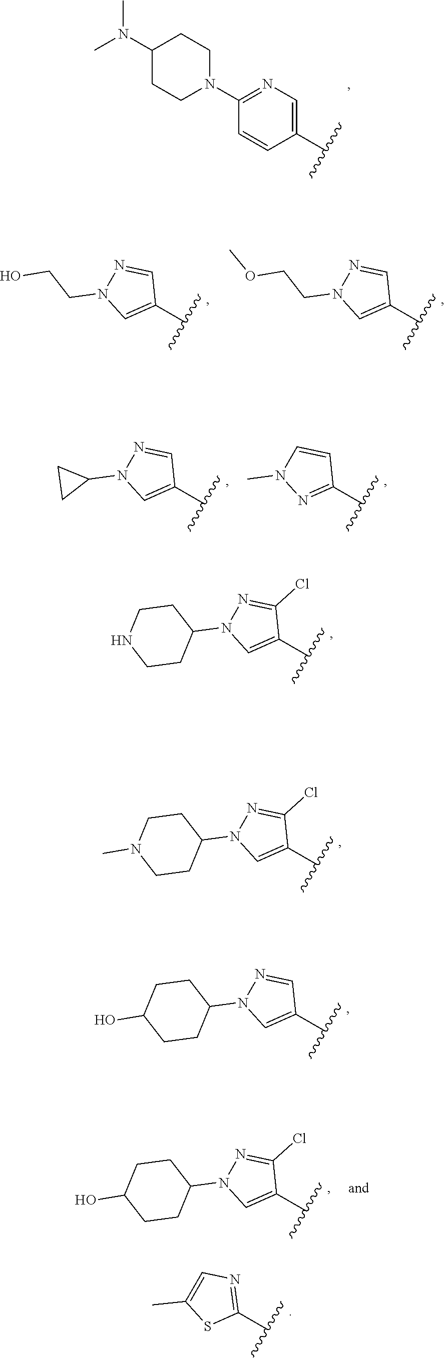

In some embodiments, R³ is selected from:

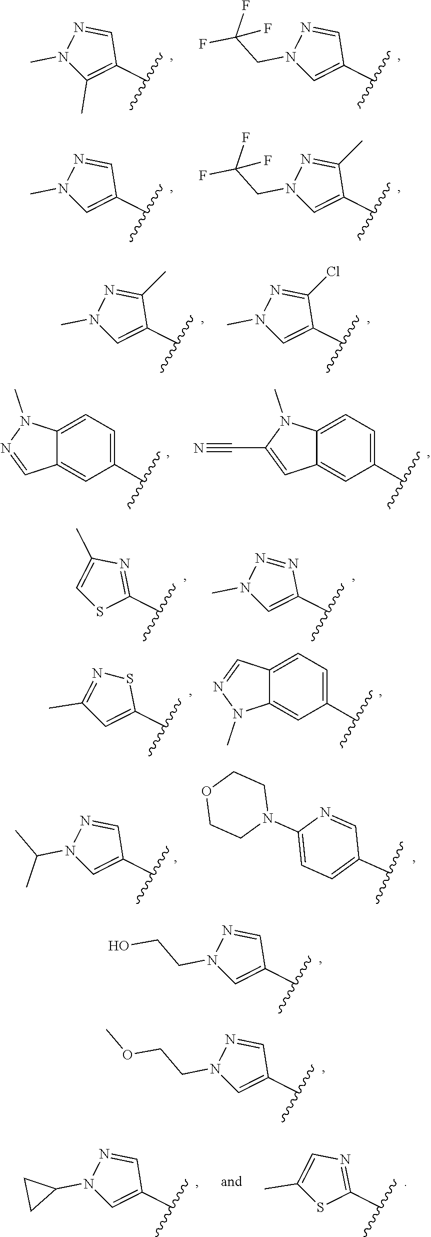

In some embodiments, R³ is selected from:

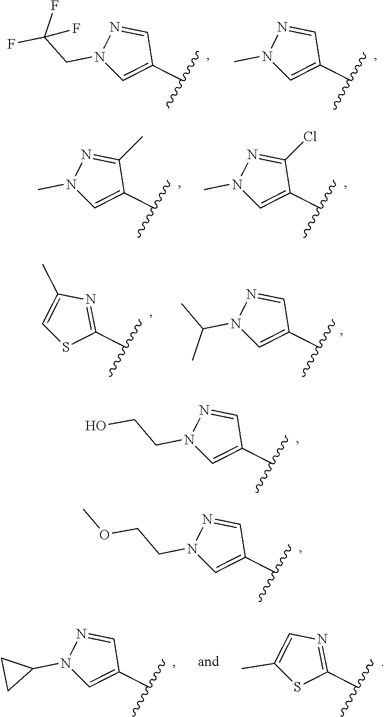

In some embodiments, R³ is

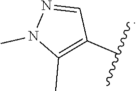 [SMILES: *c1cnn(C)c1C]

In some embodiments, R³ is

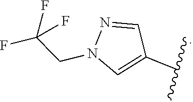 [SMILES: *c1cnn(CC(F)(F)F)c1]

In some embodiments, R³ is

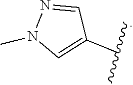 [SMILES: *c1cnn(C)c1]

In some embodiments, R³ is

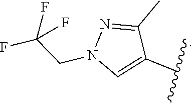 [SMILES: *c1cn(CC(F)(F)F)nc1C]

In some embodiments, R³ is

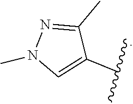 [SMILES: *c1cn(C)nc1C]

In some embodiments, R³ is

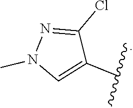 [SMILES: *c1cn(C)nc1Cl]

In some embodiments, R³ is

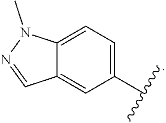 [SMILES: *c1ccc2c(cnn2C)c1]

In some embodiments, R³ is

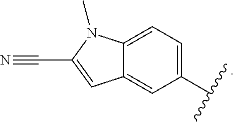 [SMILES: *c1ccc2c(c1)cc(C#N)n2C]

In some embodiments, R³ is

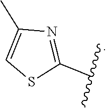 [SMILES: *c1nc(C)cs1]

In some embodiments, R³ is

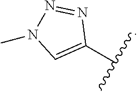 [SMILES: *c1cn(C)nn1]

In some embodiments, R³ is

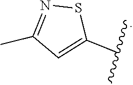 [SMILES: *c1cc(C)ns1]

In some embodiments, R³ is

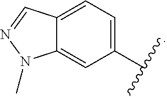 [SMILES: *c1ccc2cnn(C)c2c1]

In some embodiments, R³ is

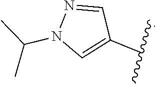 [SMILES: *c1cnn(C(C)C)c1]

In some embodiments, R³ is

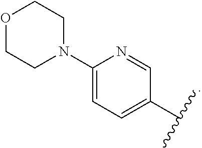 [SMILES: *c1ccc(N2CCOCC2)nc1]

In some embodiments, R³ is

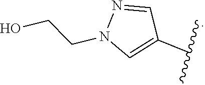 [SMILES: *c1cnn(CCO)c1]

In some embodiments, R³ is

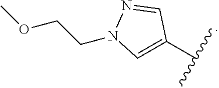 [SMILES: *c1cnn(CCOC)c1]

In some embodiments, R³ is

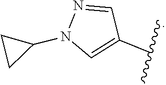 [SMILES: *c1cnn(C2CC2)c1]

In some embodiments, R³ is

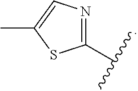 [SMILES: *c1ncc(C)s1]

In some embodiments, R³ is

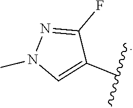 [SMILES: *c1cn(C)nc1F]

In some embodiments, R³ is

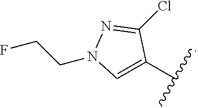 [SMILES: *c1cn(CCF)nc1Cl]

In some embodiments, R³ is

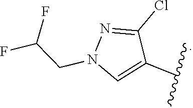 [SMILES: *c1cn(CC(F)F)nc1Cl]

In some embodiments, R³ is

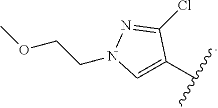 [SMILES: *c1cn(CCOC)nc1Cl]

In some embodiments, R³ is

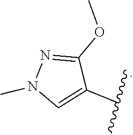 [SMILES: *c1cn(C)nc1OC]

In some embodiments, R³ is

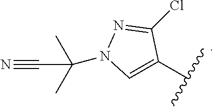 [SMILES: *c1cn(C(C)(C)C#N)nc1Cl]

In some embodiments, R³ is

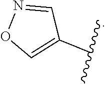 [SMILES: *c1cnoc1]

In some embodiments, R³ is

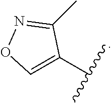 [SMILES: *c1conc1C]

In some embodiments, R³ is

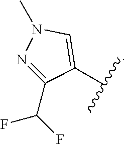 [SMILES: *c1cn(C)nc1C(F)F]

In some embodiments, R³ is

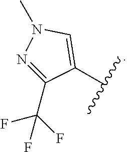 [SMILES: *c1cn(C)nc1C(F)(F)F]

In some embodiments, R³ is

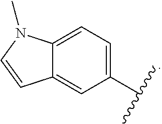 [SMILES: *c1ccc2c(ccn2C)c1]

In some embodiments, R³ is

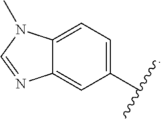 [SMILES: *c1ccc2c(c1)ncn2C]

In some embodiments, R³ is

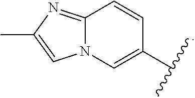 [SMILES: *c1ccc2nc(C)cn2c1]

In some embodiments, R³ is

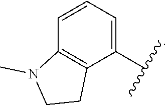 [SMILES: *c1cccc2c1CCN2C]

In some embodiments, R³ is

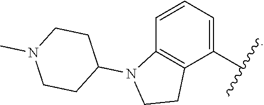 [SMILES: *c1cccc2c1CCN2C1CCN(C)CC1]

In some embodiments, R³ is

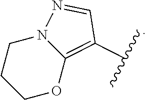 [SMILES: *c1cnn2c1OCCC2]

In some embodiments, R³ is

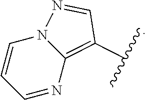 [SMILES: *c1cnn2cccnc12]

In some embodiments, R³ is

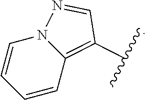 [SMILES: *c1cnn2ccccc12]

In some embodiments, R³ is

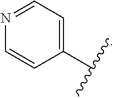 [SMILES: *c1ccncc1]

In some embodiments, R³ is

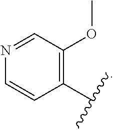 [SMILES: *c1ccncc1OC]

In some embodiments, R³ is

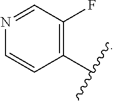 [SMILES: *c1ccncc1F]

In some embodiments, R³ is

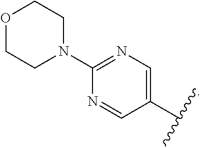 [SMILES: *c1cnc(N2CCOCC2)nc1]

In some embodiments, R³ is

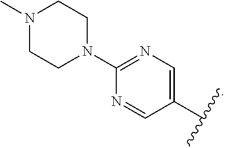 [SMILES: *c1cnc(N2CCN(C)CC2)nc1]

In some embodiments, R³ is

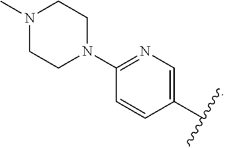 [SMILES: *c1ccc(N2CCN(C)CC2)nc1]

In some embodiments, R³ is

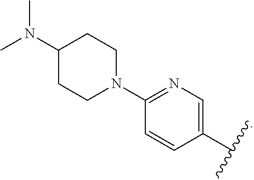 [SMILES: *c1ccc(N2CCC(N(C)C)CC2)nc1]

In some embodiments, R³ is

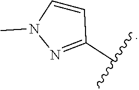 [SMILES: *c1ccn(C)n1]

In some embodiments, R³ is

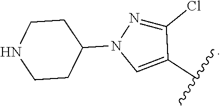 [SMILES: *c1cn(C2CCNCC2)nc1Cl]

In some embodiments, R³ is

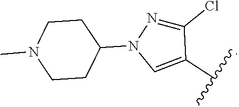 [SMILES: *c1cn(C2CCN(C)CC2)nc1Cl]

In some embodiments, R³ is

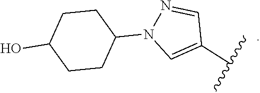 [SMILES: *c1cnn(C2CCC(O)CC2)c1]

In some embodiments, R³ is

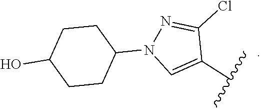 [SMILES: *c1cn(C2CCC(O)CC2)nc1Cl]

In some embodiments, each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl. In some embodiments, each R¹² is independently alkyl, heteroalkyl, haloalkyl, halo, cyano, heterocycloalkyl, —N(R¹³)₂, or cycloalkyl. In some embodiments, each R¹² is independently aryl. In some embodiments, each R¹² is independently heteroaryl. In some embodiments, each R¹² is independently alkyl. In some embodiments, each R¹² is independently heteroalkyl. In some embodiments, each R¹² is independently haloalkyl. In some embodiments, each R¹² is independently halo. In some embodiments, each R¹² is cyano. In some embodiments, each R¹² is independently alkoxy. In some embodiments, each R¹² is independently heterocycloalkyl. In some embodiments, each R¹² is independently —N(R¹³)₂. In some embodiments, each R¹² is independently —S(═O)₂NH₂. In some embodiments, each R¹² is independently —S(═O)₂alkyl. In some embodiments, each R¹² is independently —S(═O)₂aryl. In some embodiments, each R¹² is independently —S(═O)₂heteroaryl. In some embodiments, each R¹² is independently cycloalkyl. In some embodiments, each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl. In some embodiments, each R¹² is independently methyl, iso-propyl, tert-butyl, hydroxyethyl, methoxyethyl, trifluoromethyl, trifluoroethyl, chloro, cyano, morpholinyl, or cyclopropyl. In some embodiments, each R¹² is independently methyl, hydroxyethyl, methoxyethyl, trifluoroethyl, or chloro. In some embodiments, each R¹² is independently methyl or chloro. In some embodiments, each R¹² is methyl. In some embodiments, each R¹² is ethyl. In some embodiments, each R¹² is n-propyl. In some embodiments, each R¹² is iso-propyl. In some embodiments, each R¹² is n-butyl. In some embodiments, each R¹² is iso-butyl. In some embodiments, each R¹² is sec-butyl. In some embodiments, each R¹² is tert-butyl. In some embodiments, each R¹² is hydroxyethyl. In some embodiments, each R¹² is methoxyethyl. In some embodiments, each R¹² is trifluoromethyl. In some embodiments, each R¹² is trifluoroethyl. In some embodiments, each R¹² is pentafluoroethyl. In some embodiments, each R¹² is fluoro. In some embodiments, each R¹² is chloro. In some embodiments, each R¹² is azetidinyl. In some embodiments, each R¹² is oxetanyl. In some embodiments, each R¹² is pyrrolidinyl. In some embodiments, each R¹² is imidazolidinyl. In some embodiments, each R¹² is tetrahydrofuranyl. In some embodiments, each R¹² is piperidinyl. In some embodiments, each R¹² is piperazinyl. In some embodiments, each R¹² is tetrahydropyranyl. In some embodiments, each R¹² is morpholinyl. In some embodiments, each R¹² is cyclopropyl. In some embodiments, each R¹² is cyclobutyl. In some embodiments, each R¹² is cyclopentyl. In some embodiments, each R¹² is cyclohexyl.

In some embodiments, each R¹³ is independently alkyl, cycloalkyl, aryl, or heteroaryl. In some embodiments, each R¹³ is independently alkyl or cycloalkyl. In some embodiments, each R¹³ is independently alkyl. In some embodiments, each R¹³ is independently cycloalkyl. In some embodiments, each R¹³ is independently aryl. In some embodiments, each R¹³ is independently heteroaryl. In some embodiments, each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹³ is independently methyl, ethyl, iso-propyl, tert-butyl, cyclopropyl, cyclopentyl, or cyclohexyl. In some embodiments, each R¹³ is independently methyl, cyclopropyl, or cyclohexyl. In some embodiments, each R¹³ is methyl. In some embodiments, each R¹³ is ethyl. In some embodiments, each R¹³ is n-propyl. In some embodiments, each R¹³ is iso-propyl. In some embodiments, each R¹³ is n-butyl. In some embodiments, each R¹³ is iso-butyl. In some embodiments, each R¹³ is sec-butyl. In some embodiments, each R¹³ is tert-butyl. In some embodiments, each R¹³ is cyclopropyl. In some embodiments, each R¹³ is cyclobutyl. In some embodiments, each R¹³ is cyclopentyl. In some embodiments, each R¹³ is cyclohexyl.

In some embodiments, the aryl, heteroaryl, heterocycloalkyl, or cycloalkyl of R¹² is unsubstituted. In some embodiments, the aryl, heteroaryl, heterocycloalkyl, or cycloalkyl of R¹² is substituted with 1 or 2 R¹⁴. In some embodiments, the aryl, heteroaryl, heterocycloalkyl, or cycloalkyl of R¹² is substituted with 1 R¹⁴. In some embodiments, the aryl, heteroaryl, heterocycloalkyl, or cycloalkyl of R¹² is substituted with 2 R¹⁴.

In some embodiments, each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy. In some embodiments, each R¹⁴ is independently alkyl, cycloalkyl, heterocycloalkyl, halo, cyano, —N(R¹⁵)₂, or alkoxy. In some embodiments, each R¹⁴ is independently aryl. In some embodiments, each R¹⁴ is independently heteroaryl. In some embodiments, each R¹⁴ is independently alkyl. In some embodiments, each R¹⁴ is independently cycloalkyl. In some embodiments, each R¹⁴ is independently heterocycloalkyl. In some embodiments, each R¹⁴ is independently halo. In some embodiments, each R¹⁴ is independently heteroalkyl. In some embodiments, each R¹⁴ is independently haloalkyl. In some embodiments, each R¹⁴ is cyano. In some embodiments, each R¹⁴ is hydroxy. In some embodiments, each R¹⁴ is amino. In some embodiments, each R¹⁴ is independently —N(R¹⁵)₂. In some embodiments, each R¹⁴ is independently-S(═O)₂alkyl. In some embodiments, each R¹⁴ is independently —S(═O)₂aryl. In some embodiments, each R¹⁴ is independently —S(═O)₂heteroaryl. In some embodiments, each R¹⁴ is independently alkoxy. In some embodiments, each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂, methoxy, ethoxy, or trifluoromethoxy. In some embodiments, each R¹⁴ is independently methyl, ethyl, iso-propyl, tert-butyl, pyrrolidinyl, piperidinyl, morpholinyl, fluoro, chloro, —N(R¹⁵)₂, or methoxy. In some embodiments, each R¹⁴ is methyl. In some embodiments, each R¹⁴ is ethyl. In some embodiments, each R¹⁴ is n-propyl. In some embodiments, each R¹⁴ is iso-propyl. In some embodiments, each R¹⁴ is n-butyl. In some embodiments, each R¹⁴ is iso-butyl. In some embodiments, each R¹⁴ is sec-butyl. In some embodiments, each R¹⁴ is tert-butyl. In some embodiments, each R¹⁴ is cyclopropyl. In some embodiments, each R¹⁴ is cyclobutyl. In some embodiments, each R¹⁴ is cyclopentyl. In some embodiments, each R¹⁴ is cyclohexyl. In some embodiments, each R¹⁴ is azetidinyl. In some embodiments, each R¹⁴ is oxetanyl. In some embodiments, each R¹⁴ is pyrrolidinyl. In some embodiments, each R¹⁴ is imidazolidinyl. In some embodiments, each R¹⁴ is tetrahydrofuranyl. In some embodiments, each R¹⁴ is piperidinyl. In some embodiments, each R¹⁴ is piperazinyl. In some embodiments, each R¹⁴ is tetrahydropyranyl. In some embodiments, each R¹⁴ is morpholinyl. In some embodiments, each R¹⁴ is fluoro. In some embodiments, each R¹⁴ is chloro. In some embodiments, each R¹⁴ is methoxy. In some embodiments, each R¹⁴ is ethoxy. In some embodiments, each R¹⁴ is trifluoromethoxy.

In some embodiments, each R¹⁵ is independently alkyl, cycloalkyl, aryl, or heteroaryl. In some embodiments, each R¹⁵ is independently alkyl or cycloalkyl. In some embodiments, each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl. In some embodiments, each R¹⁵ is methyl. In some embodiments, each R¹⁵ is ethyl. In some embodiments, each R¹⁵ is n-propyl. In some embodiments, each R¹⁵ is iso-propyl. In some embodiments, each R¹⁵ is n-butyl. In some embodiments, each R¹⁵ is iso-butyl. In some embodiments, each R¹⁵ is sec-butyl. In some embodiments, each R¹⁵ is tert-butyl. In some embodiments, each R¹⁵ is cyclopropyl. In some embodiments, each R¹⁵ is cyclobutyl. In some embodiments, each R¹⁵ is cyclopentyl. In some embodiments, each R¹⁵ is cyclohexyl.

In some embodiments, the compound of Formula I is selected from:

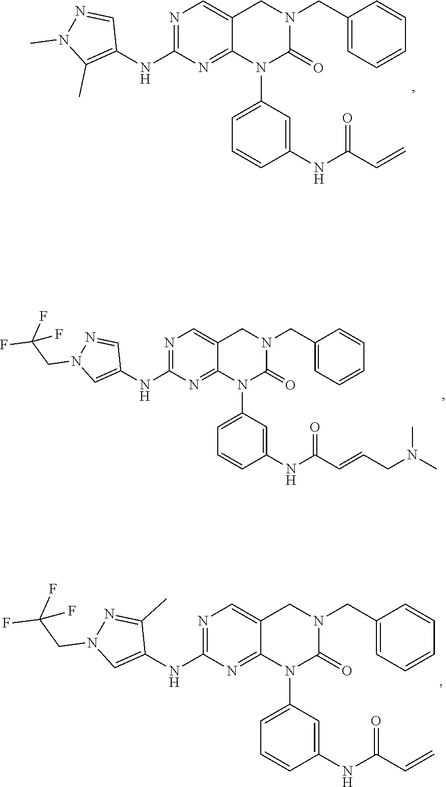 [SMILES: C=CC(=O)Nc1cccc(N2C(=O)N(Cc3ccccc3)Cc3cnc(Nc4cnn(C)c4C)nc32)c1 ; "," ; CN(C)C/C=C/C(=O)Nc1cccc(N2C(=O)N(Cc3ccccc3)Cc3cnc(Nc4cnn(CC(F)(F)F)c4)nc32)c1 ; "," ; C=CC(=O)Nc1cccc(N2C(=O)N(Cc3ccccc3)Cc3cnc(Nc4cn(CC(F)(F)F)nc4C)nc32)c1 ; ","]

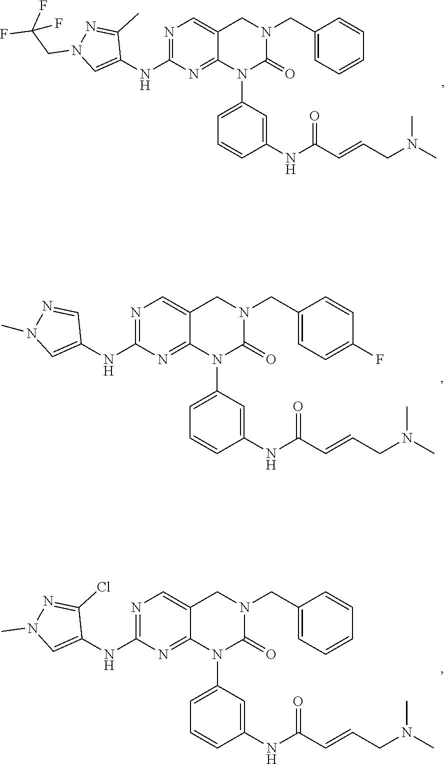

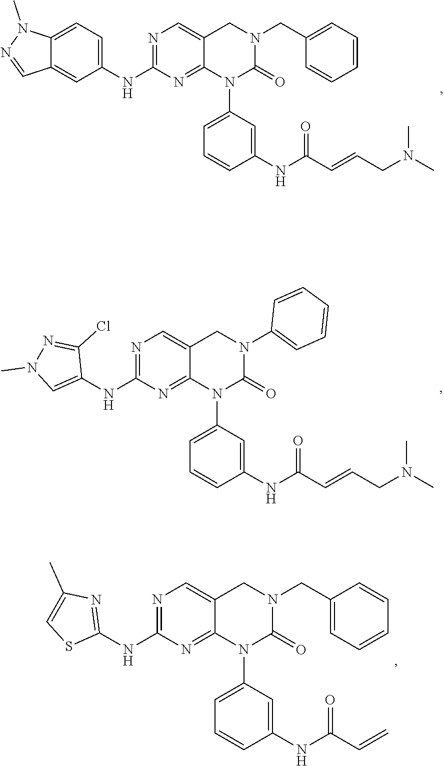

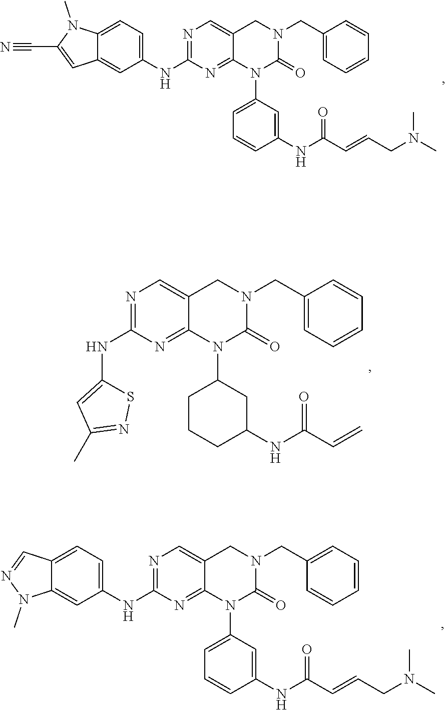

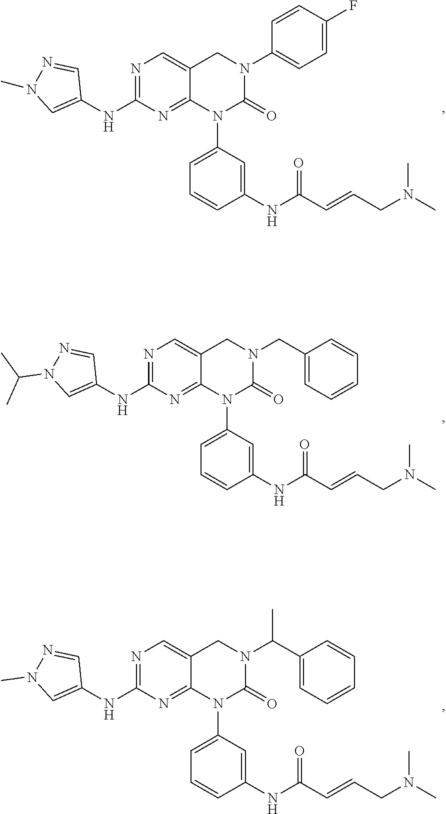

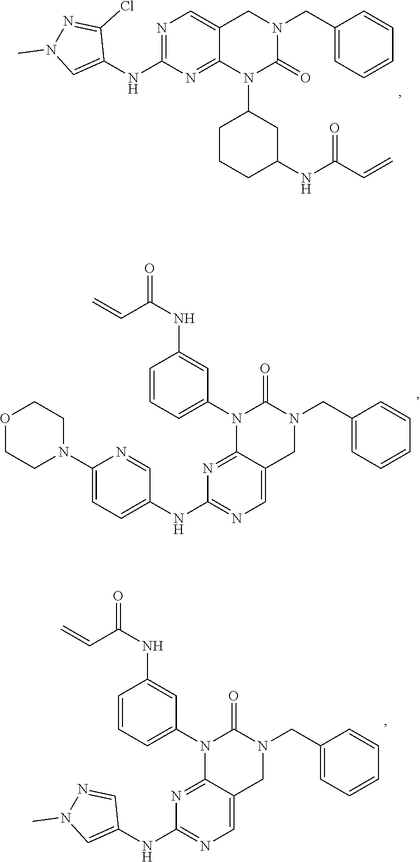 [SMILES: C=CC(=O)NC1CCCC(N2C(=O)N(Cc3ccccc3)Cc3cnc(Nc4cn(C)nc4Cl)nc32)C1 ; "," ; C=CC(=O)Nc1cccc(N2C(=O)N(Cc3ccccc3)Cc3cnc(Nc4ccc(N5CCOCC5)nc4)nc32)c1 ; "," ; C=CC(=O)Nc1cccc(N2C(=O)N(Cc3ccccc3)Cc3cnc(Nc4cnn(C)c4)nc32)c1 ; ","]

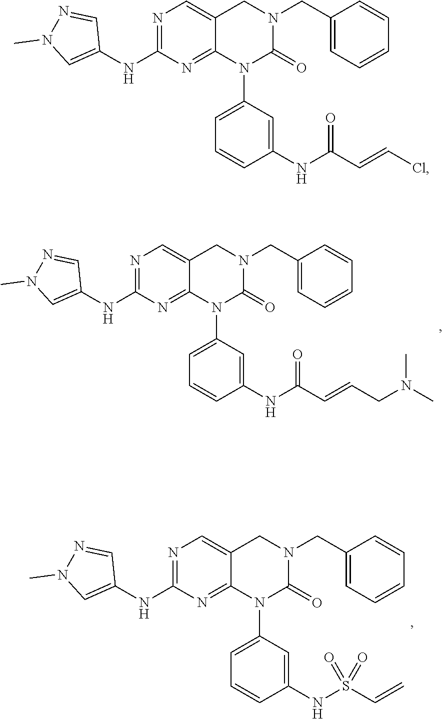

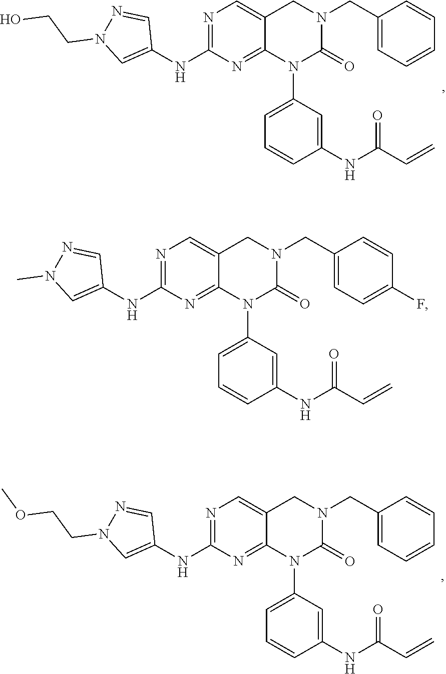 [SMILES: C=CC(=O)Nc1cccc(N2C(=O)N(Cc3ccccc3)Cc3cnc(Nc4cnn(CCO)c4)nc32)c1 ; "," ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; ","]

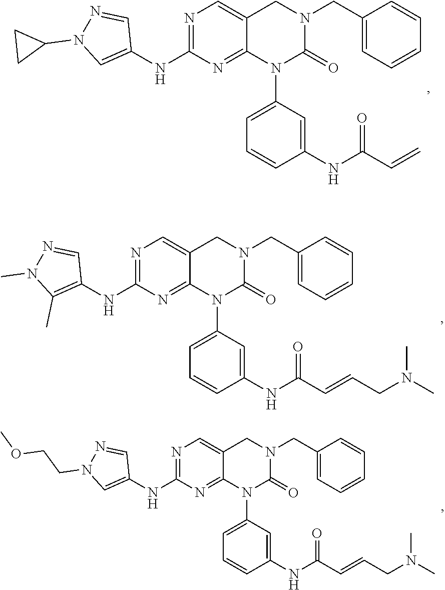

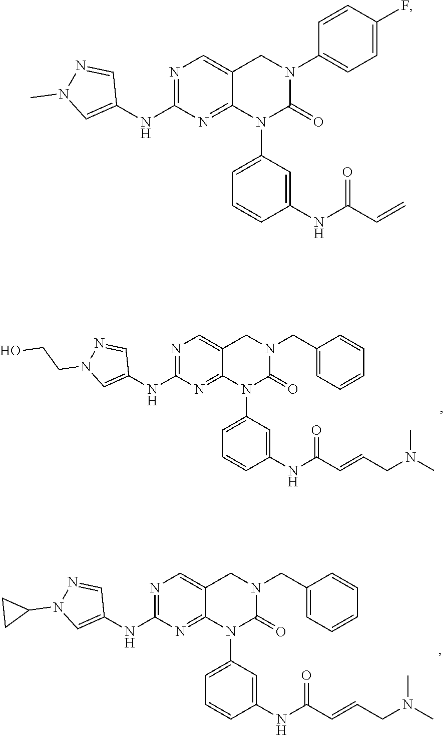 [SMILES: C=CC(=O)Nc1cccc(N2C(=O)N(c3ccc(F)cc3)Cc3cnc(Nc4cnn(C)c4)nc32)c1 ; CN(C)C/C=C/C(=O)Nc1cccc(N2C(=O)N(Cc3ccccc3)Cc3cnc(Nc4cnn(CCO)c4)nc32)c1 ; "," ; CN(C)C/C=C/C(=O)Nc1cccc(N2C(=O)N(Cc3ccccc3)Cc3cnc(Nc4cnn(C5CC5)c4)nc32)c1 ; ","]

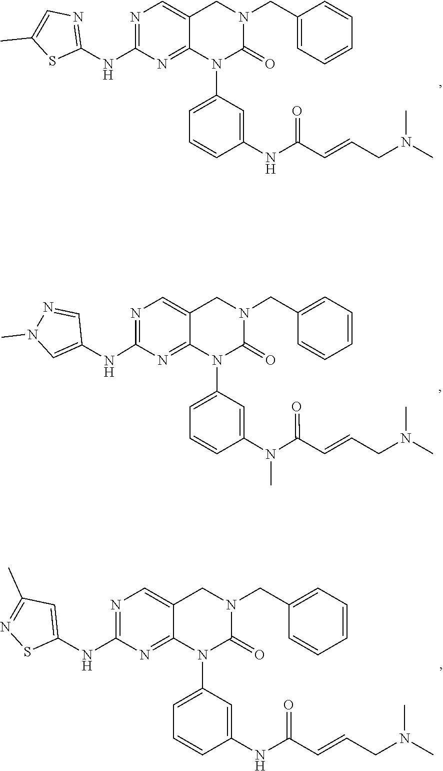

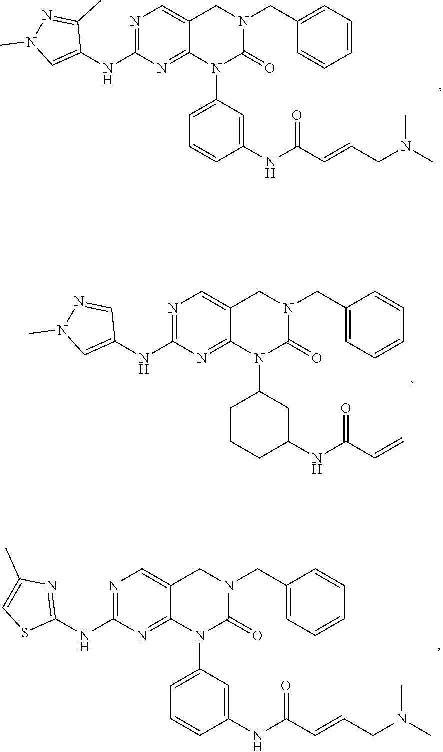

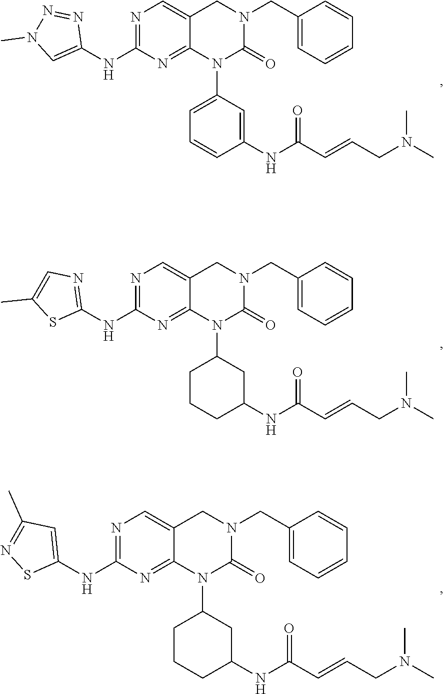

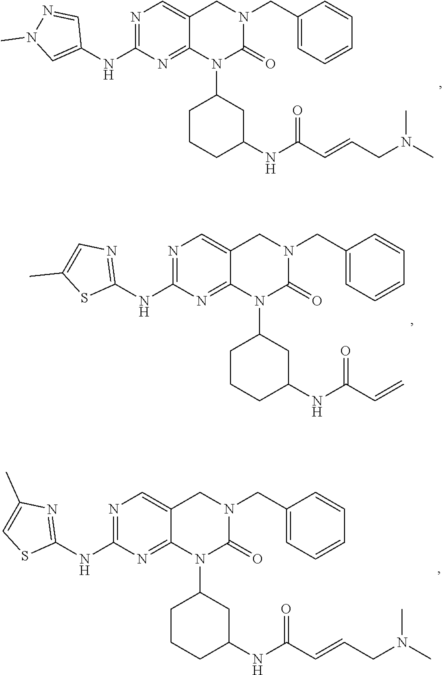

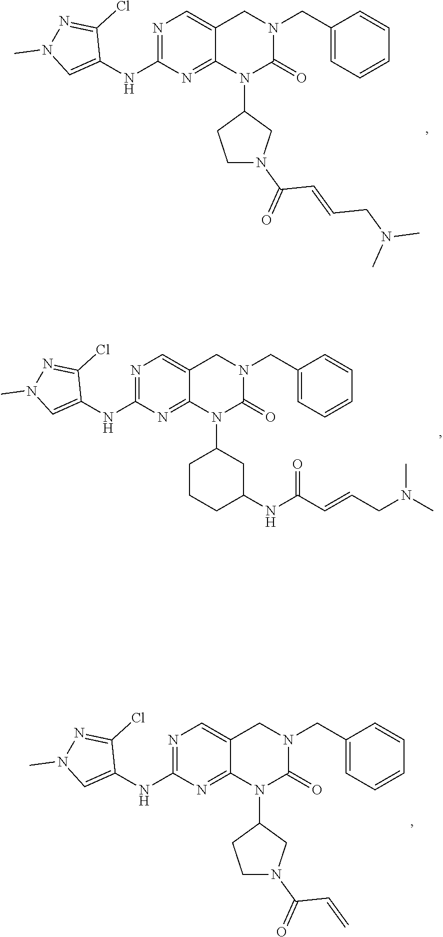

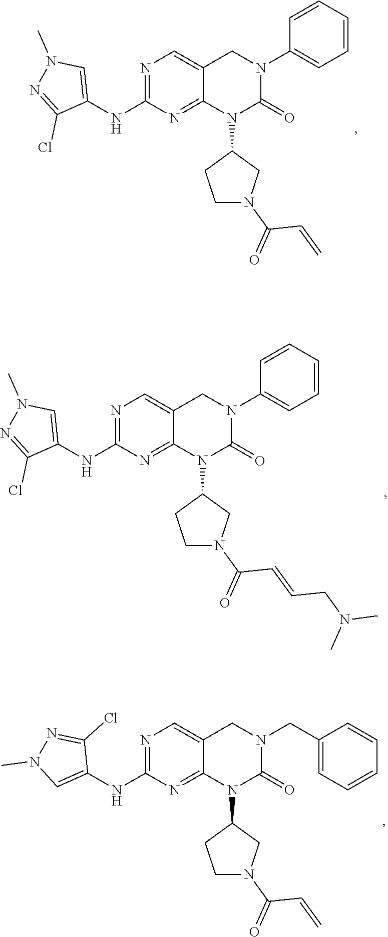

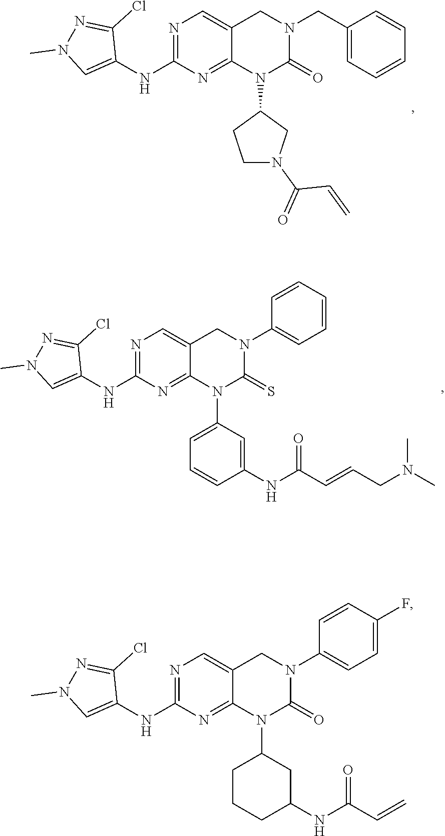

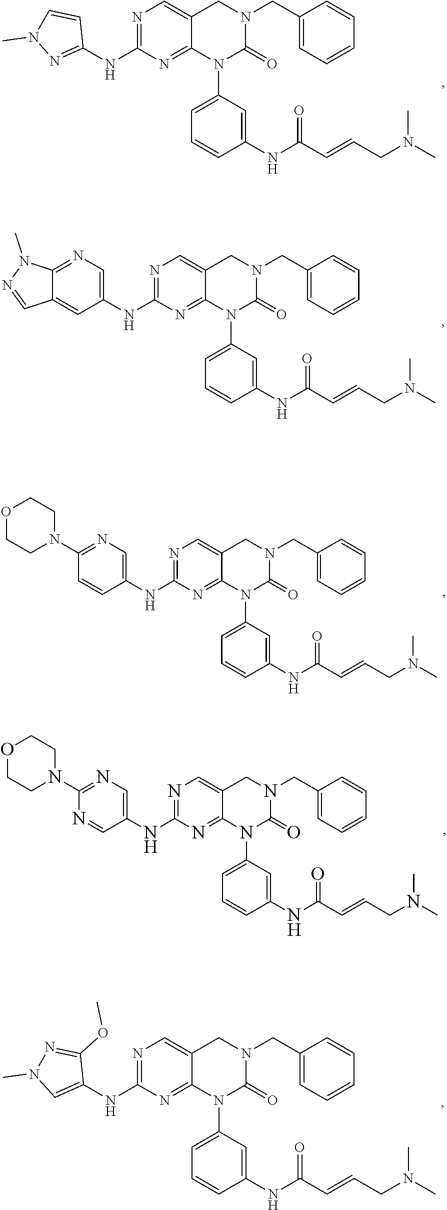

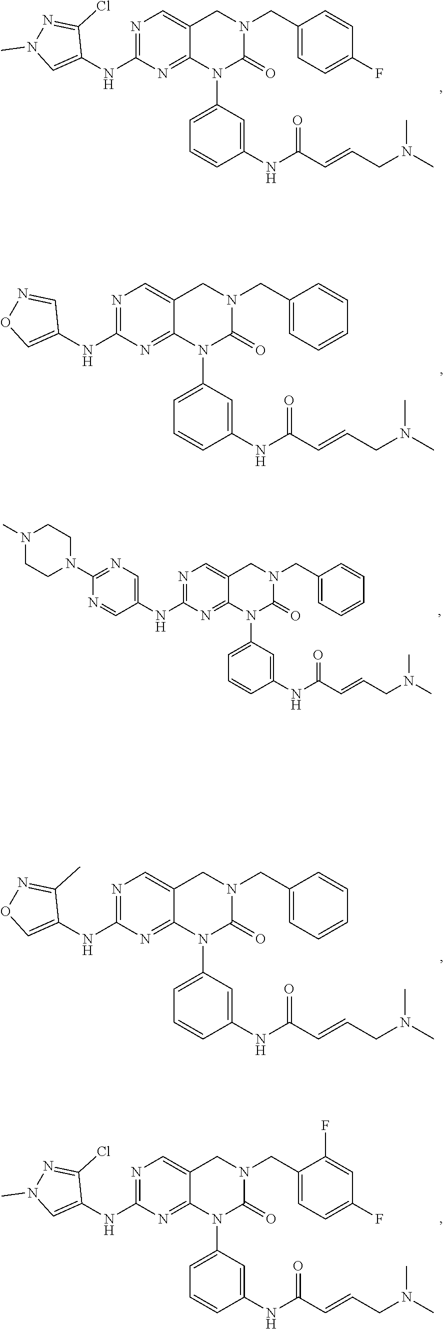

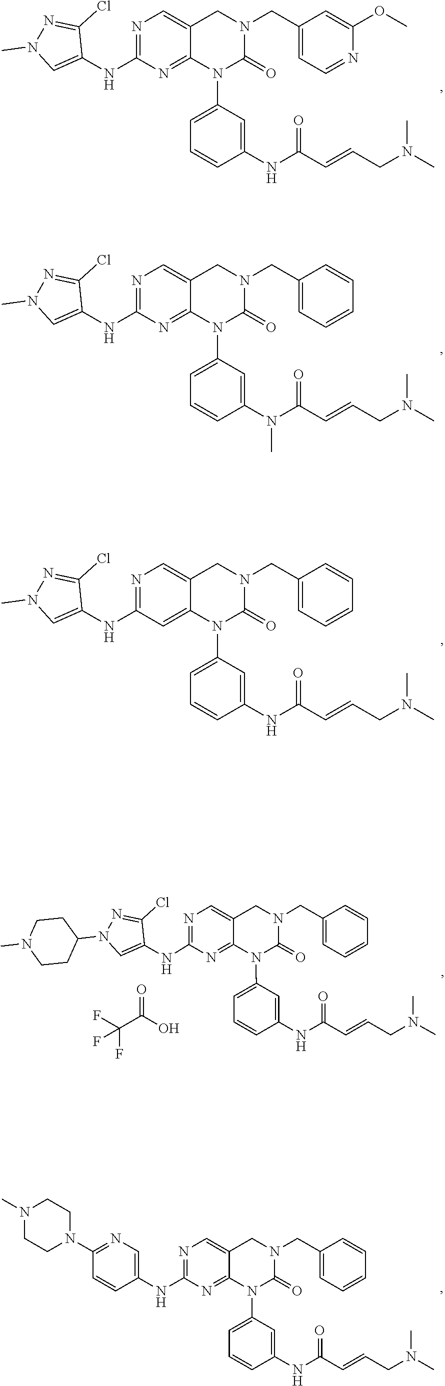

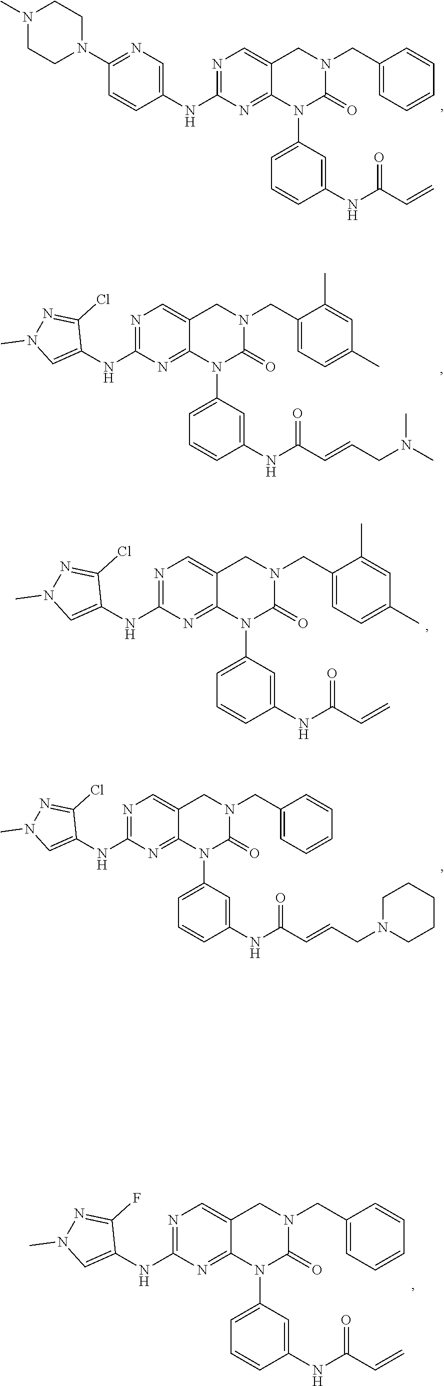

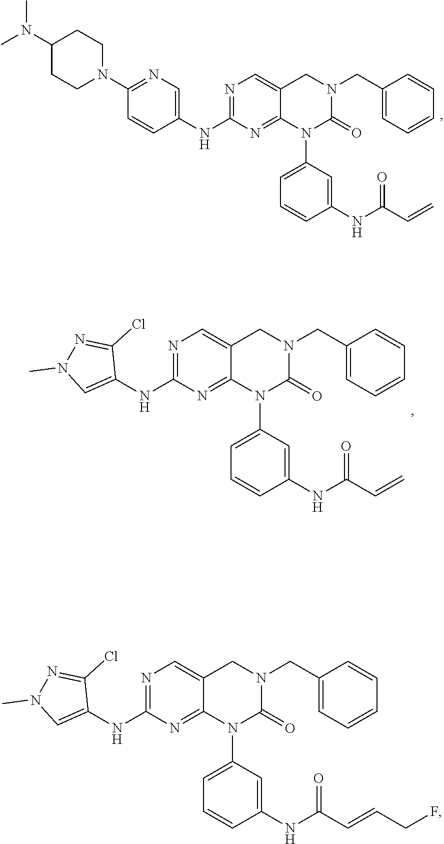 [SMILES: C=CC(=O)Nc1cccc(N2C(=O)N(Cc3ccccc3)Cc3cnc(Nc4ccc(N5CCC(N(C)C)CC5)nc4)nc32)c1 ; "," ; C=CC(=O)Nc1cccc(N2C(=O)N(Cc3ccccc3)Cc3cnc(Nc4cn(C)nc4Cl)nc32)c1 ; "," ; Cn1cc(Nc2ncc3c(n2)N(c2cccc(NC(=O)/C=C/CF)c2)C(=O)N(Cc2ccccc2)C3)c(Cl)n1]

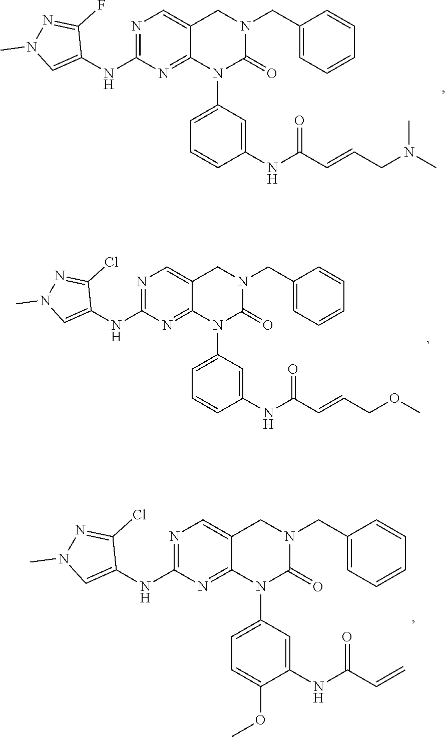

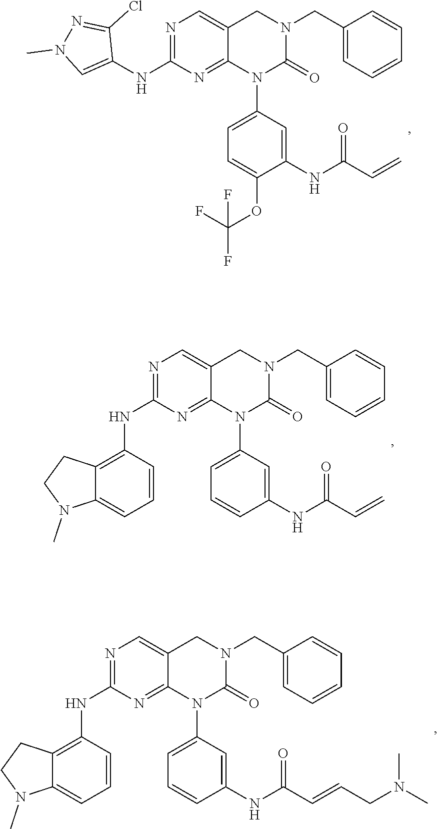 [SMILES: C=CC(=O)Nc1cc(N2C(=O)N(Cc3ccccc3)Cc3cnc(Nc4cn(C)nc4Cl)nc32)ccc1OC(F)(F)F ; "," ; C=CC(=O)Nc1cccc(N2C(=O)N(Cc3ccccc3)Cc3cnc(Nc4cccc5c4CCN5C)nc32)c1 ; "," ; CN(C)C/C=C/C(=O)Nc1cccc(N2C(=O)N(Cc3ccccc3)Cc3cnc(Nc4cccc5c4CCN5C)nc32)c1 ; ","]

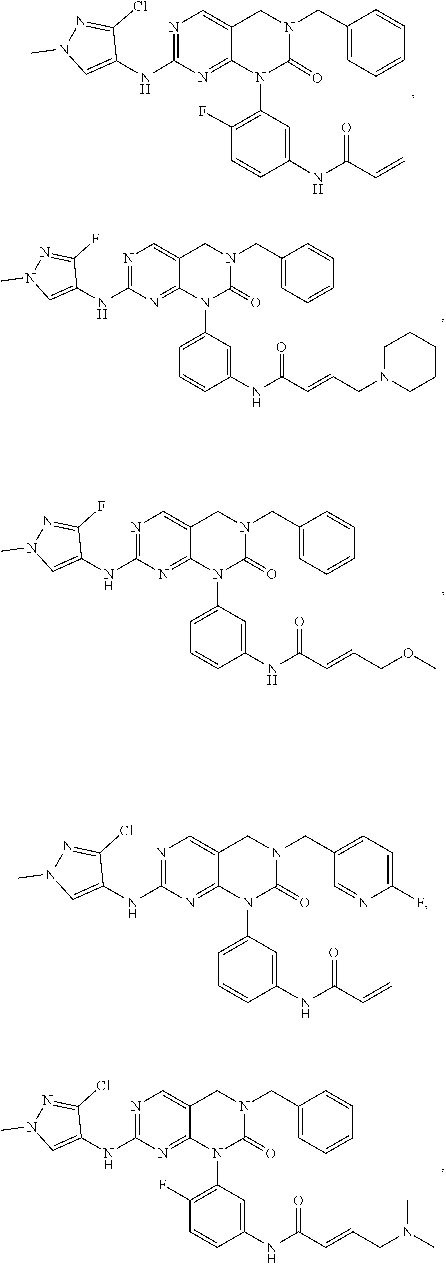

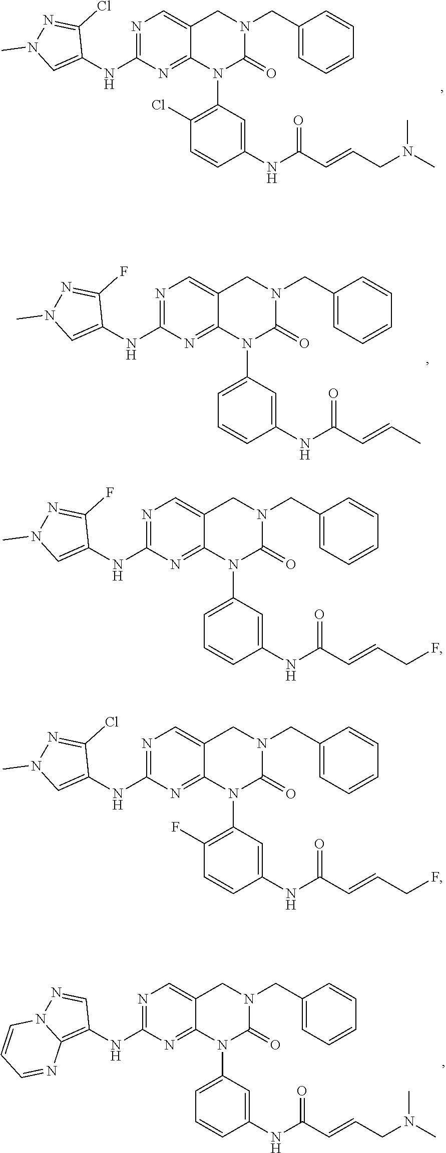

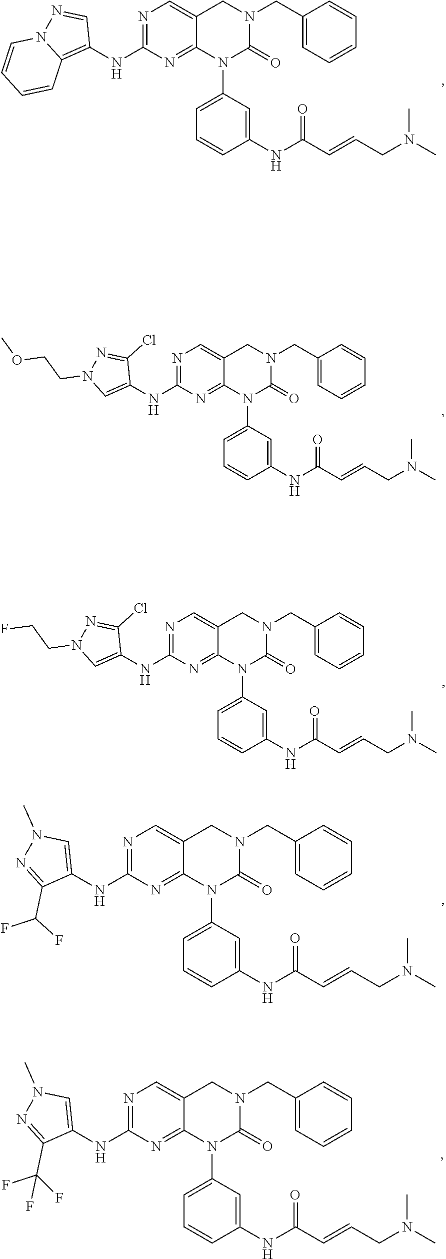

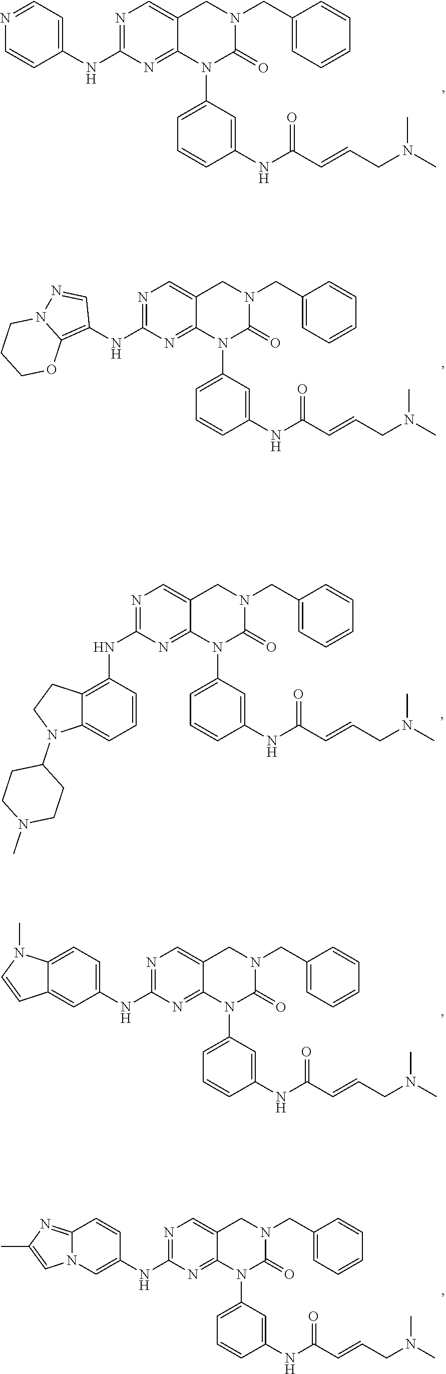

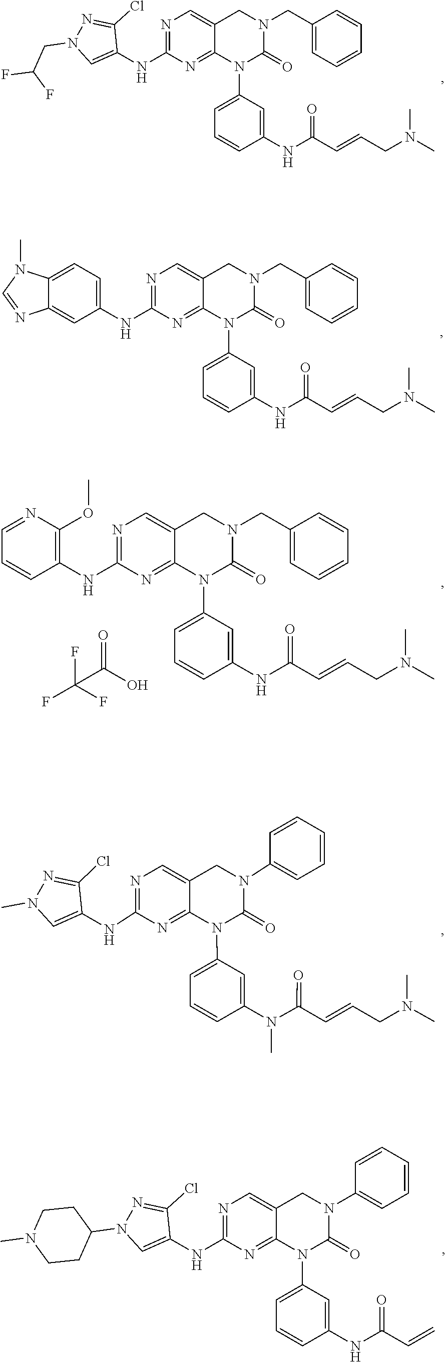

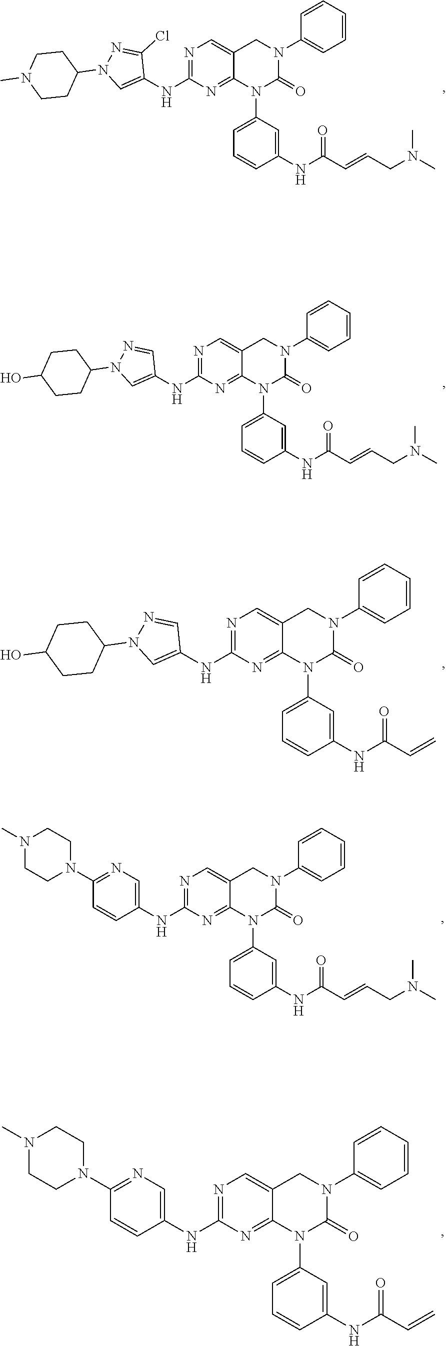

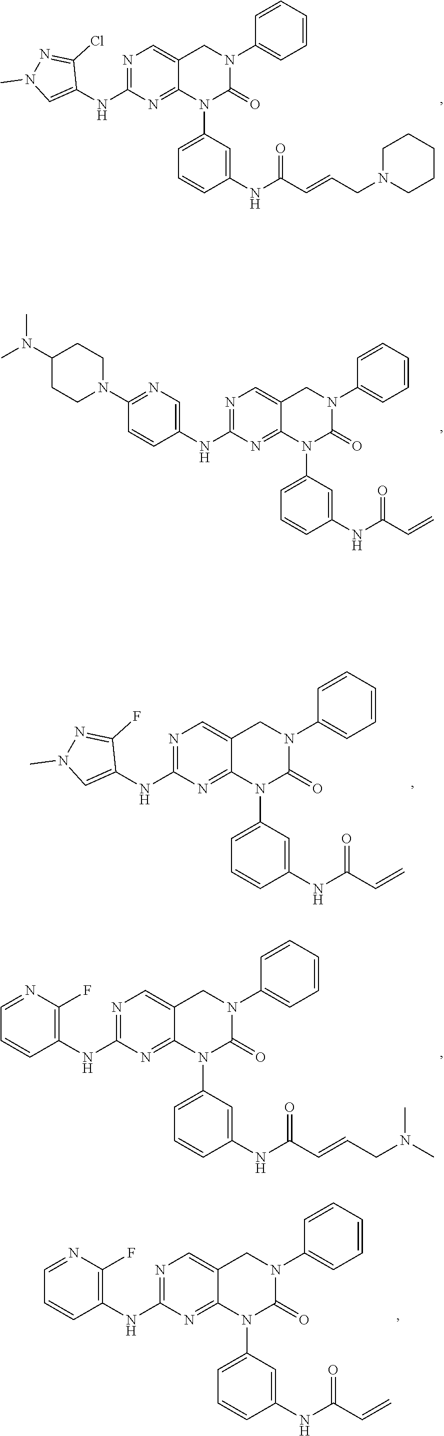

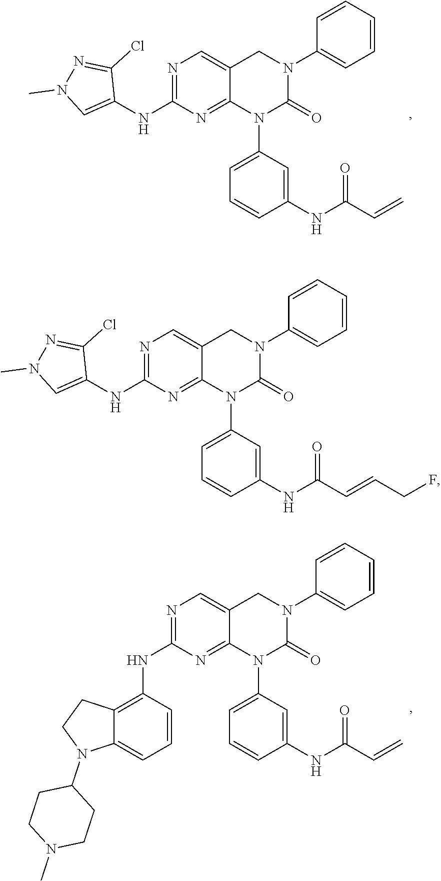

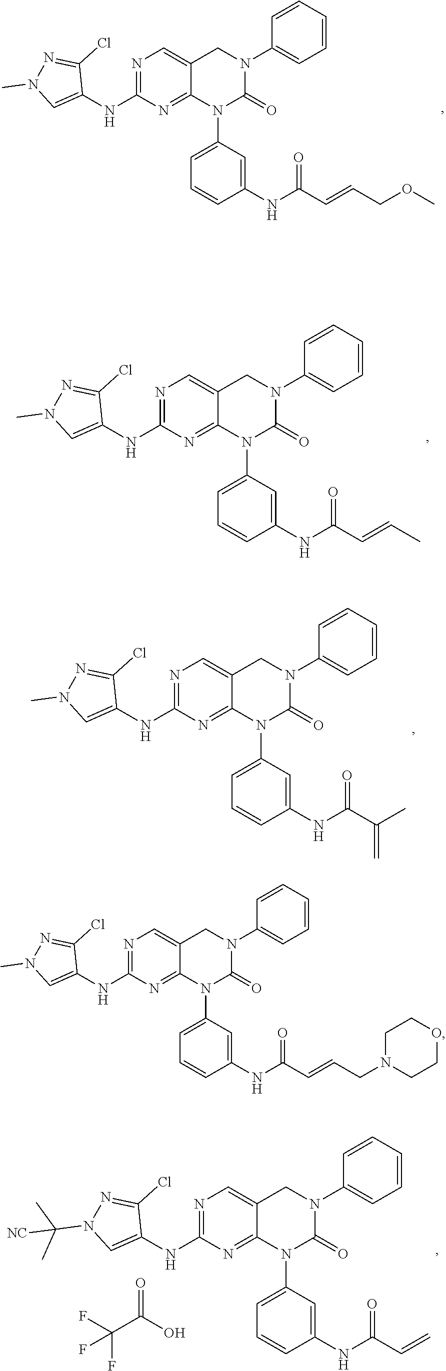

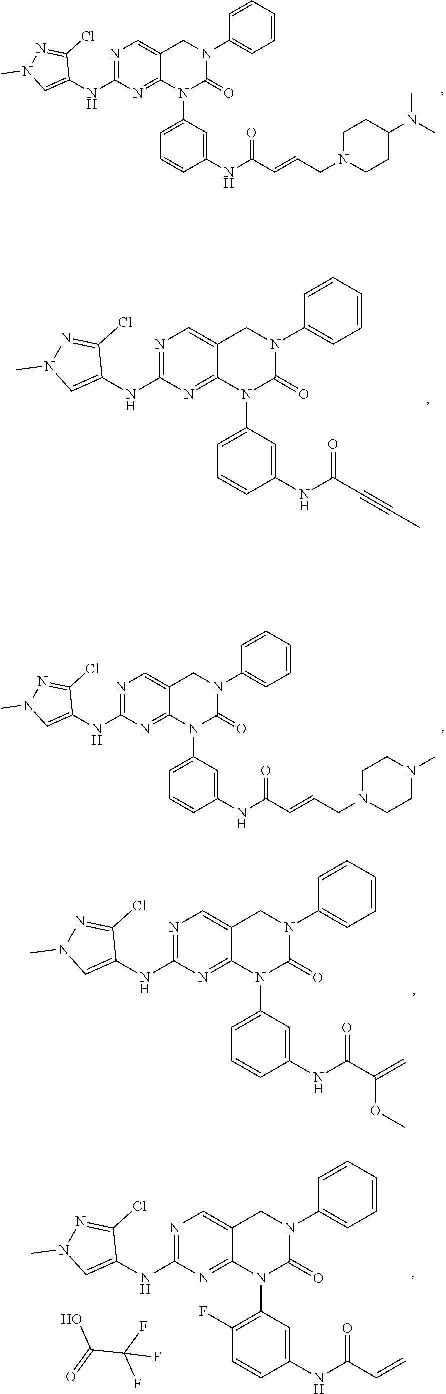

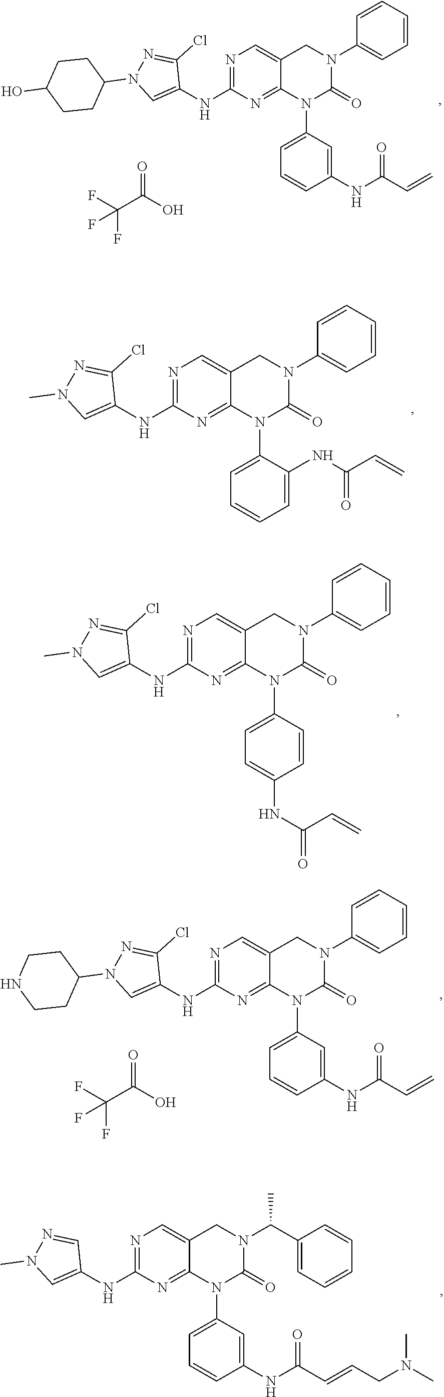

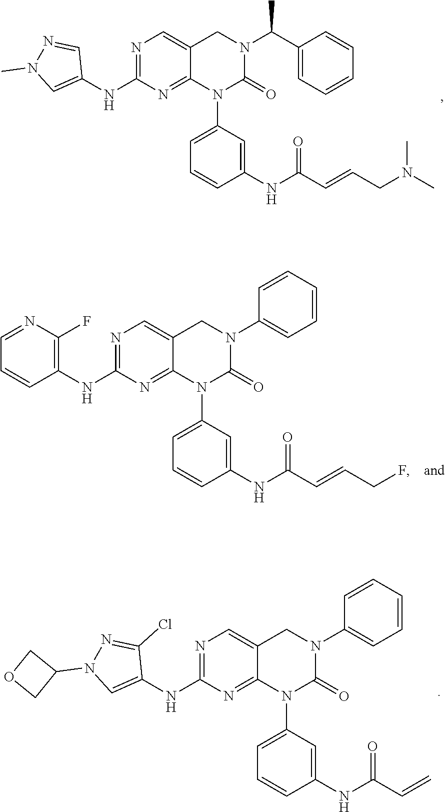

In some embodiments, the compound of Formula I is selected from:

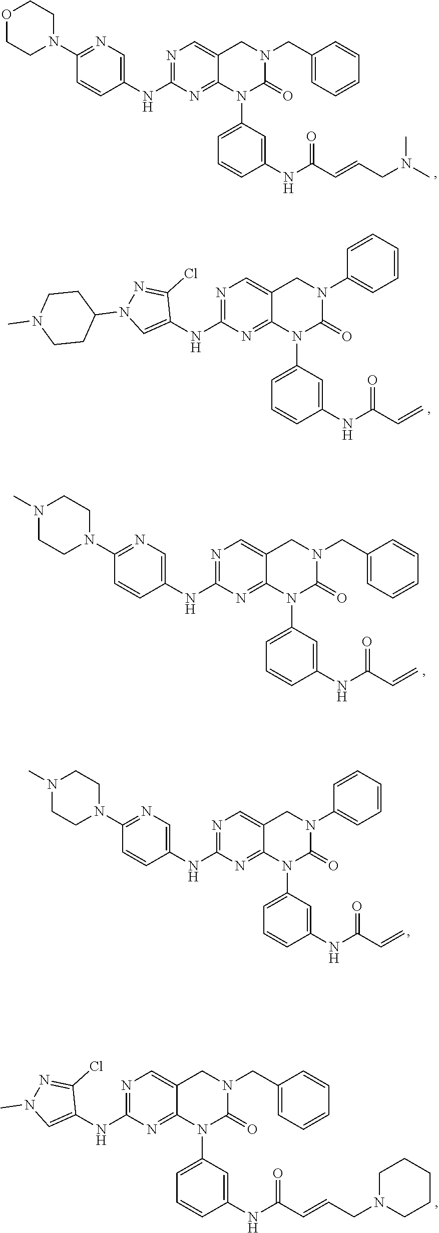

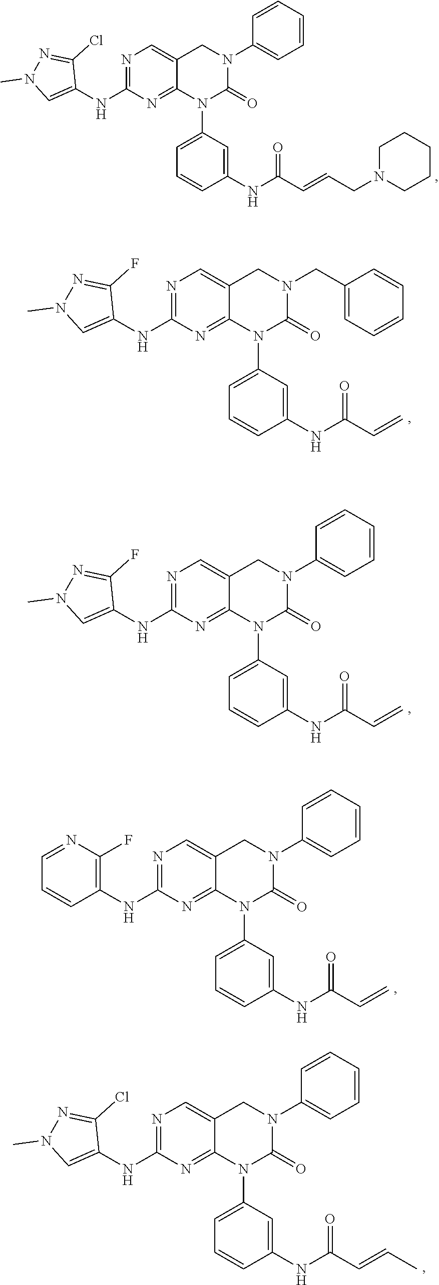

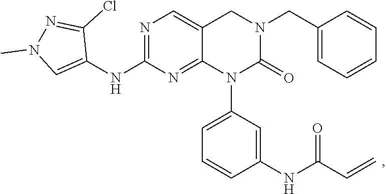 [SMILES: C=CC(=O)Nc1cccc(N2C(=O)N(Cc3ccccc3)Cc3cnc(Nc4cn(C)nc4Cl)nc32)c1]

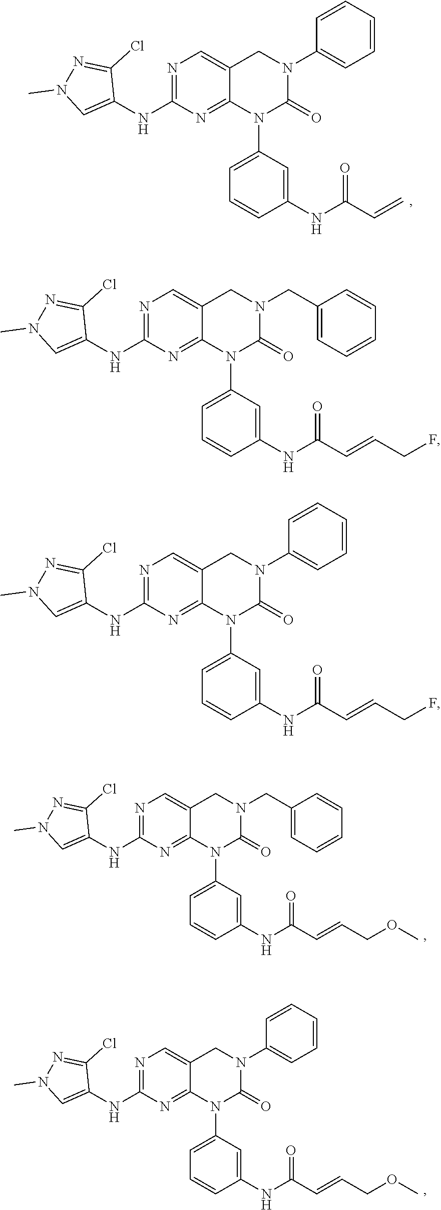

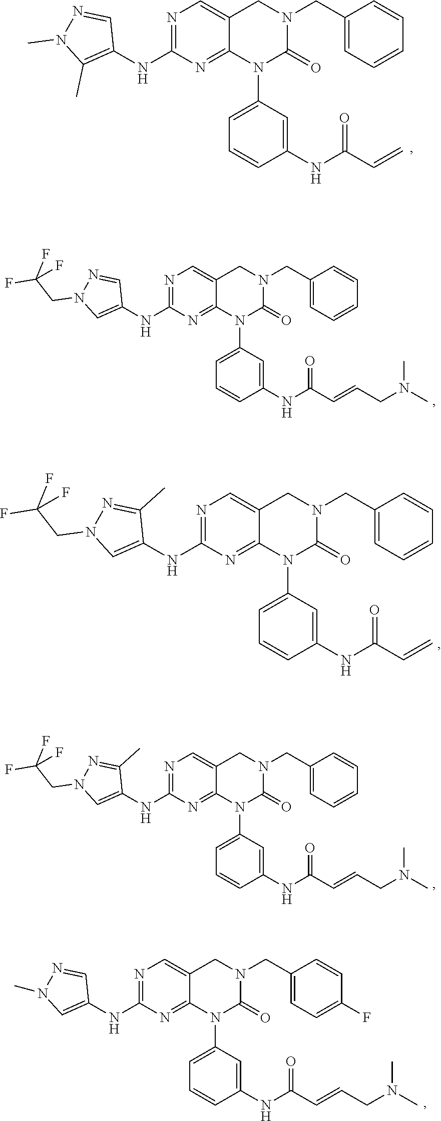

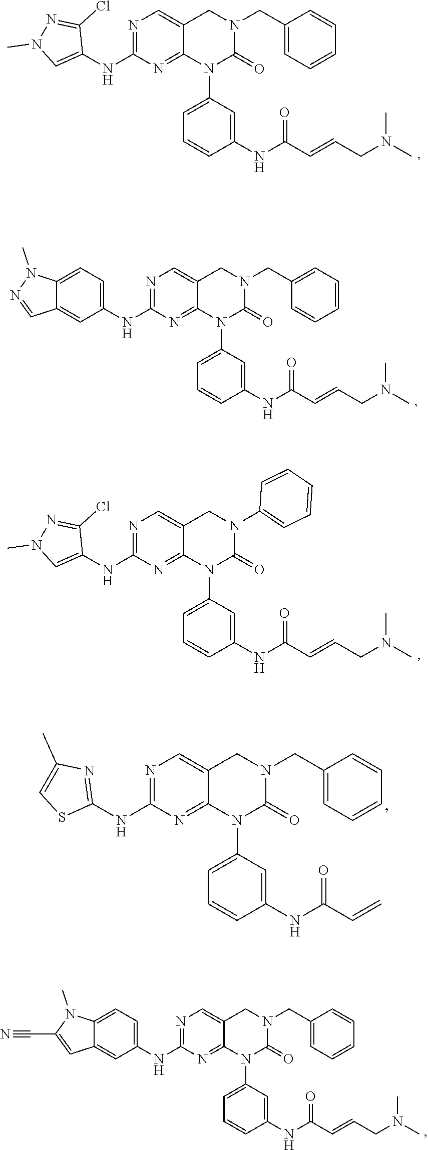

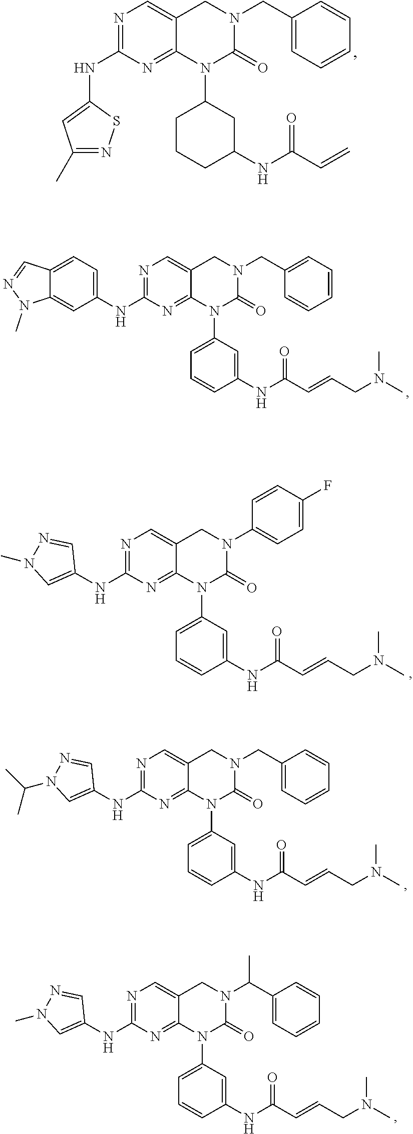

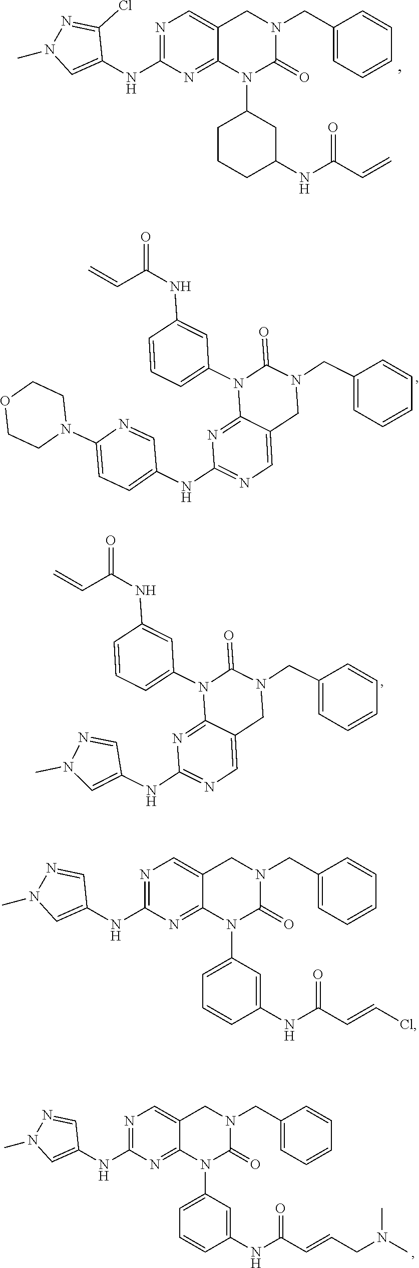

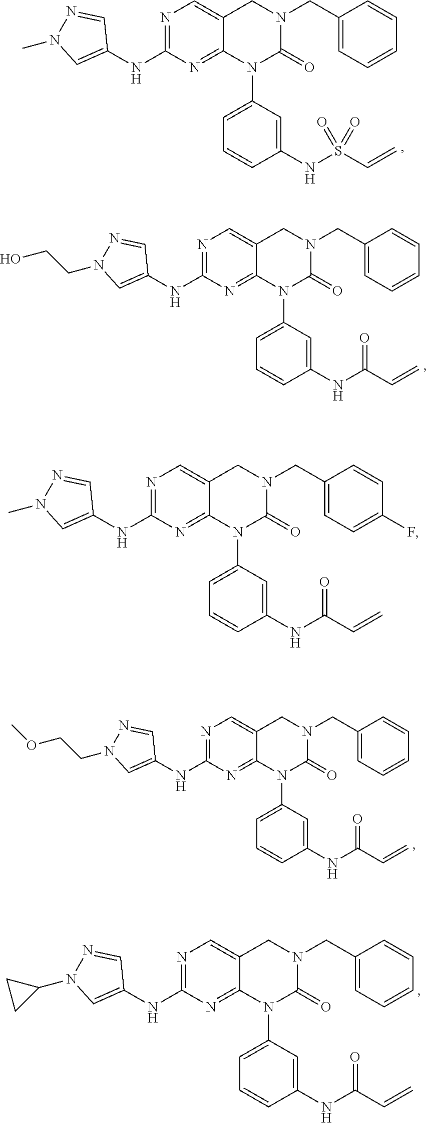 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; C=CC(=O)Nc1cccc(N2C(=O)N(Cc3ccccc3)Cc3cnc(Nc4cnn(C5CC5)c4)nc32)c1]

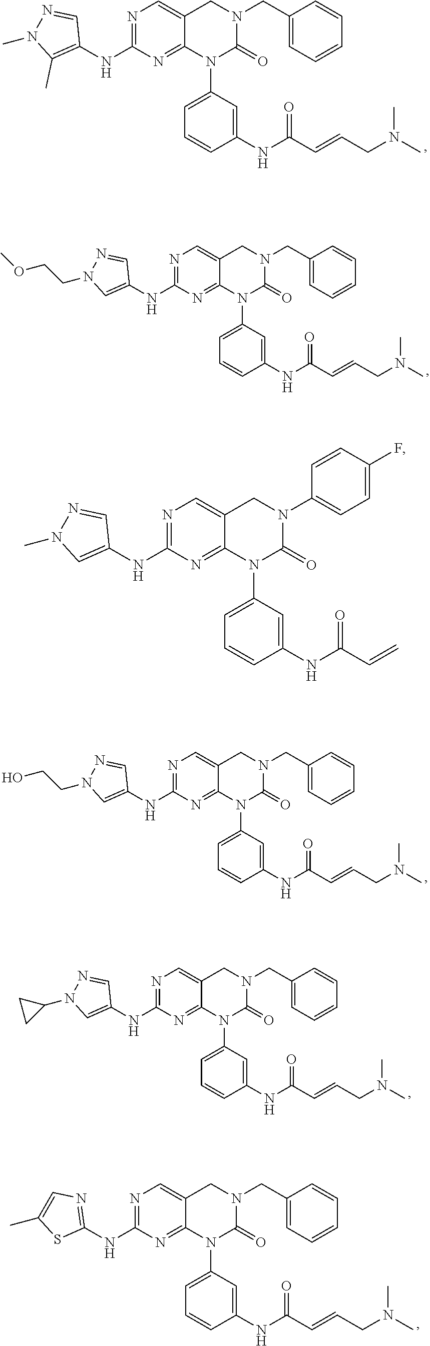

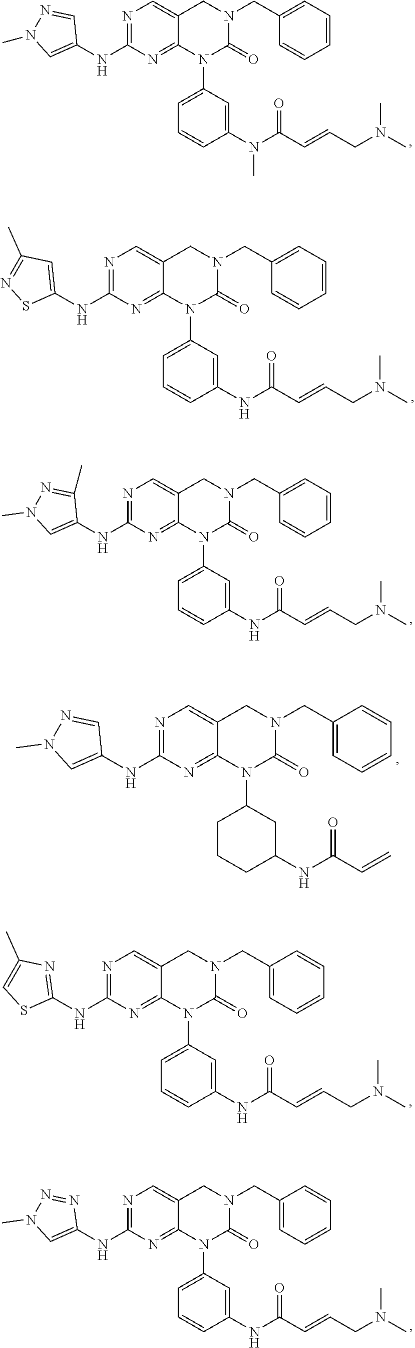

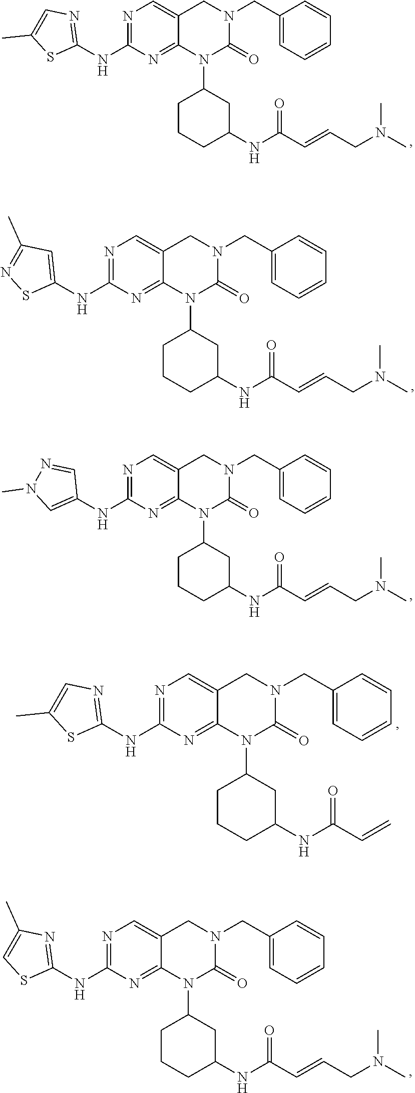

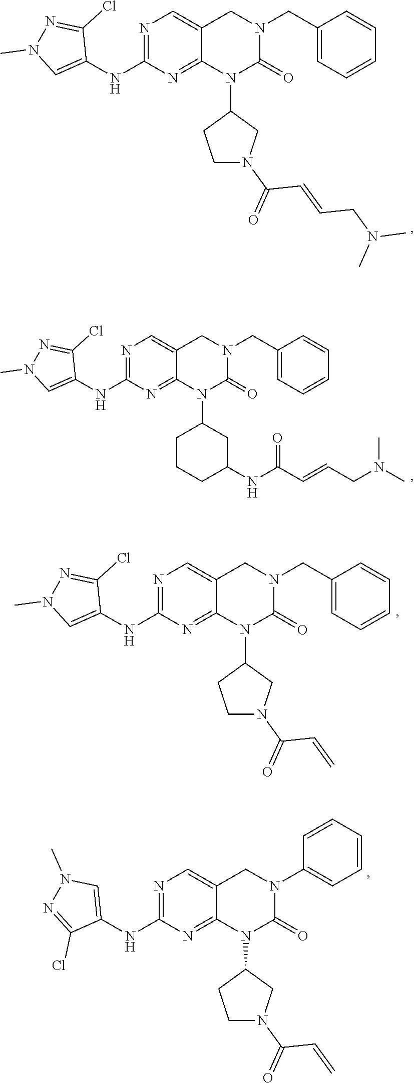

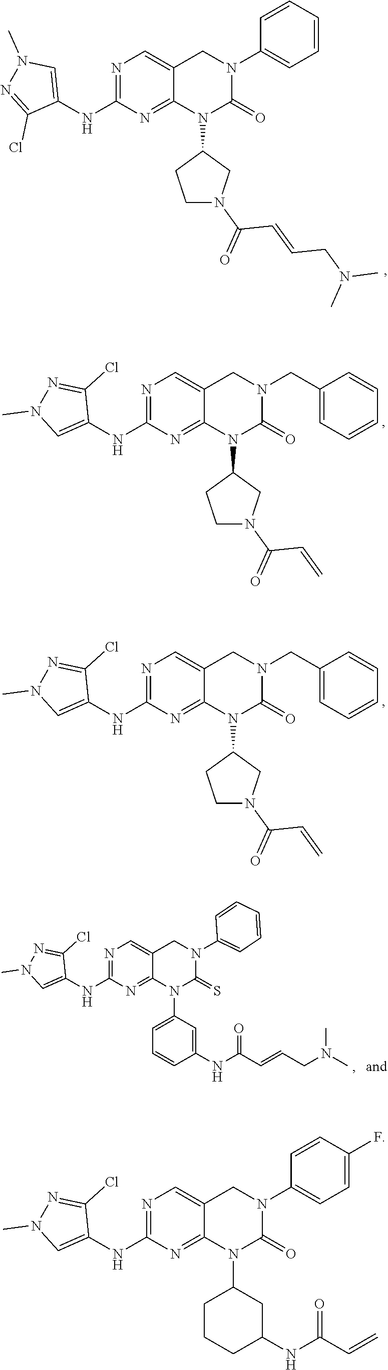

In some embodiments, the compound of Formula I is selected from:

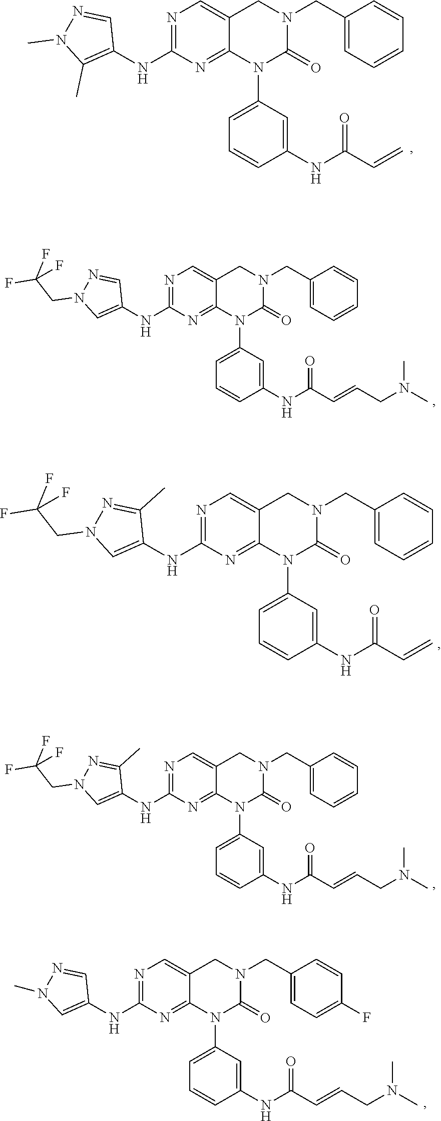

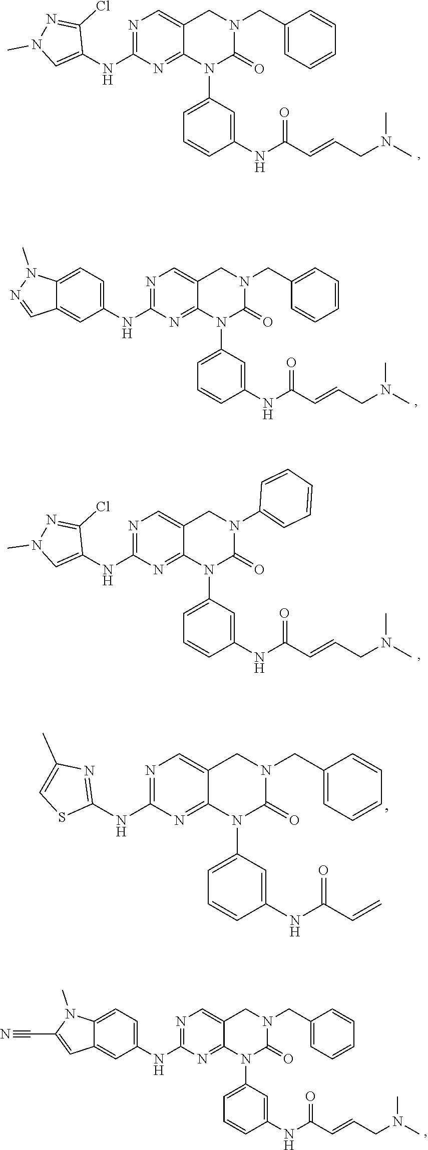

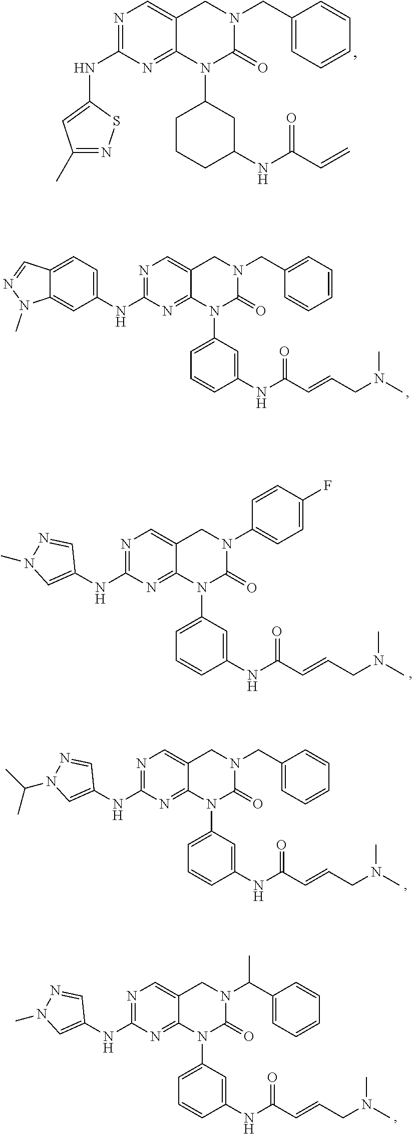

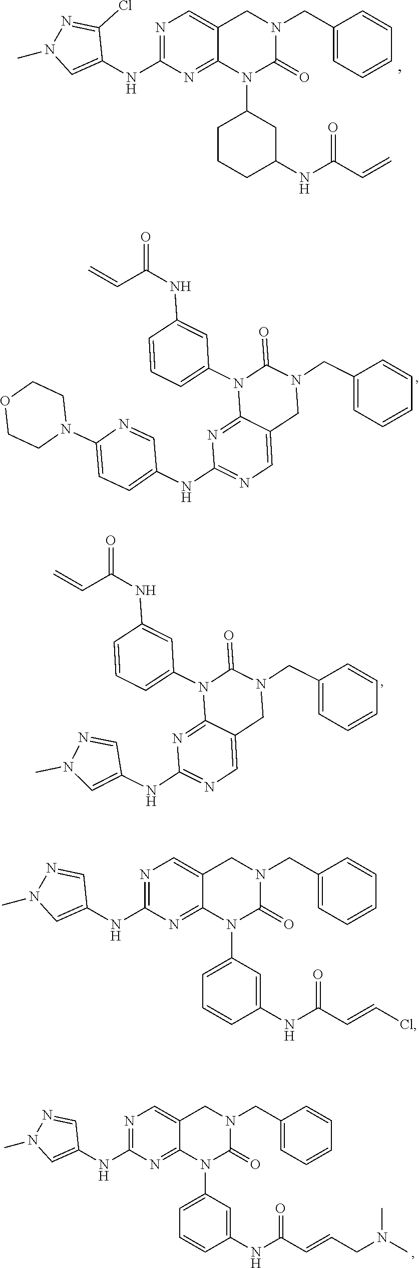

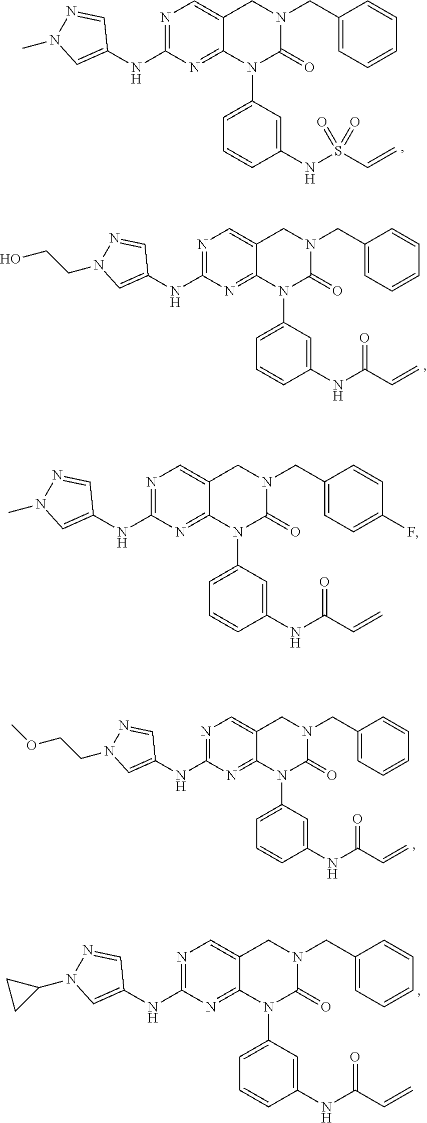 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; C=CC(=O)Nc1cccc(N2C(=O)N(Cc3ccccc3)Cc3cnc(Nc4cnn(C5CC5)c4)nc32)c1]

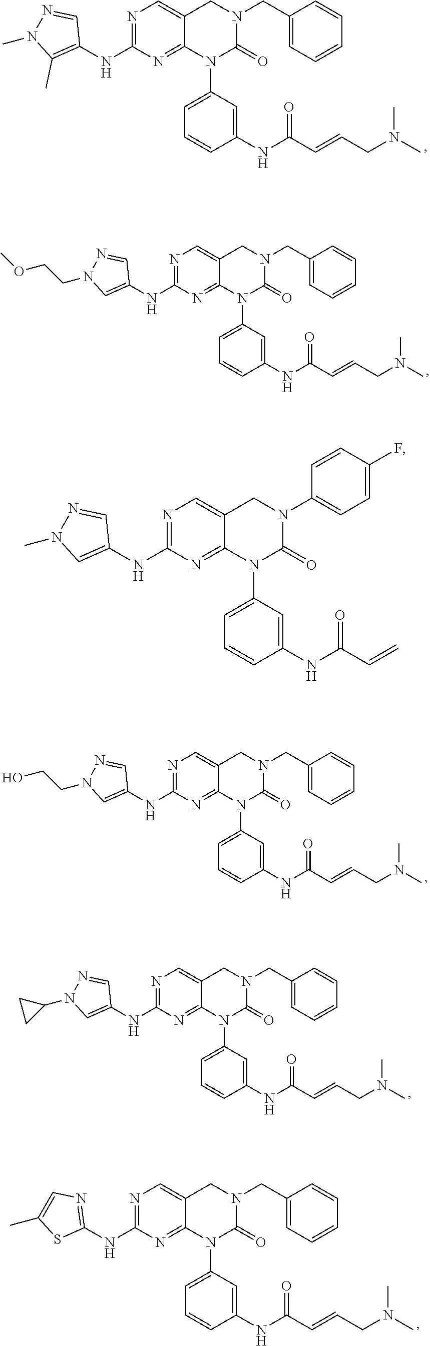

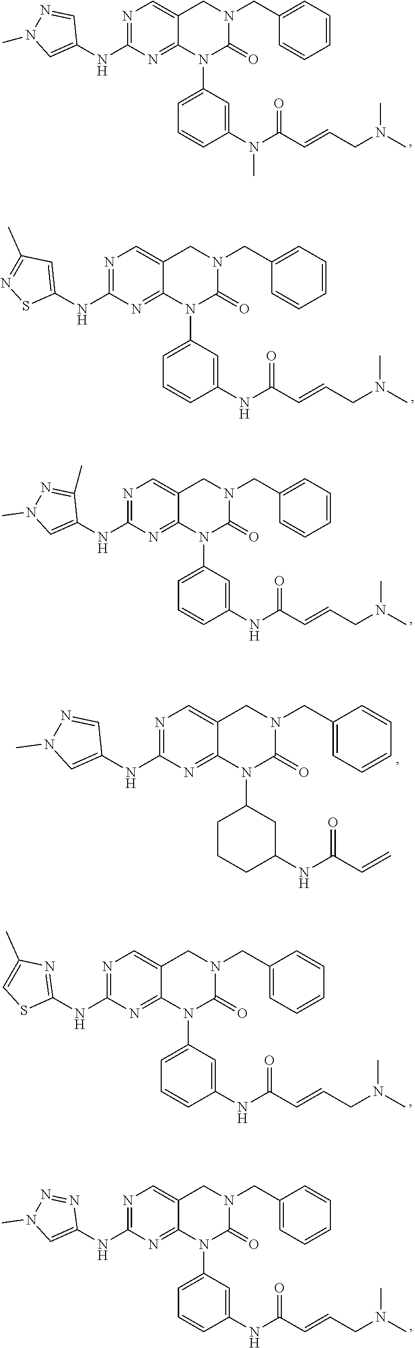

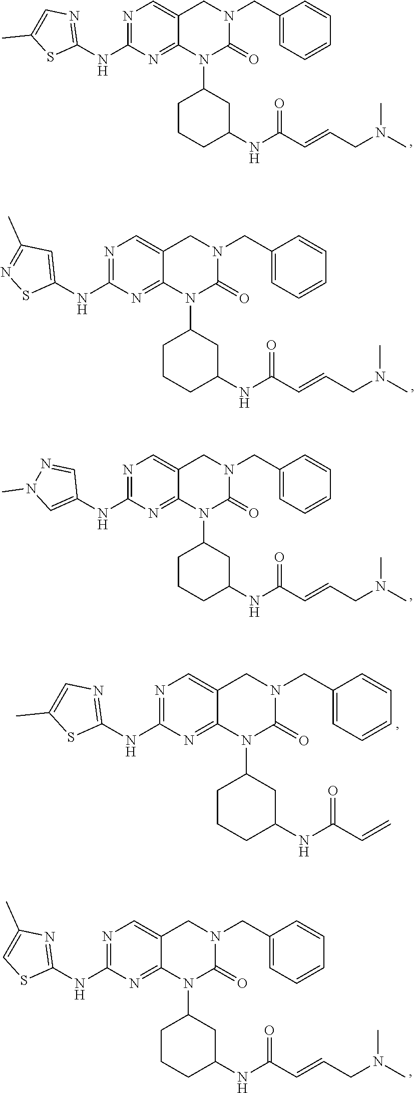

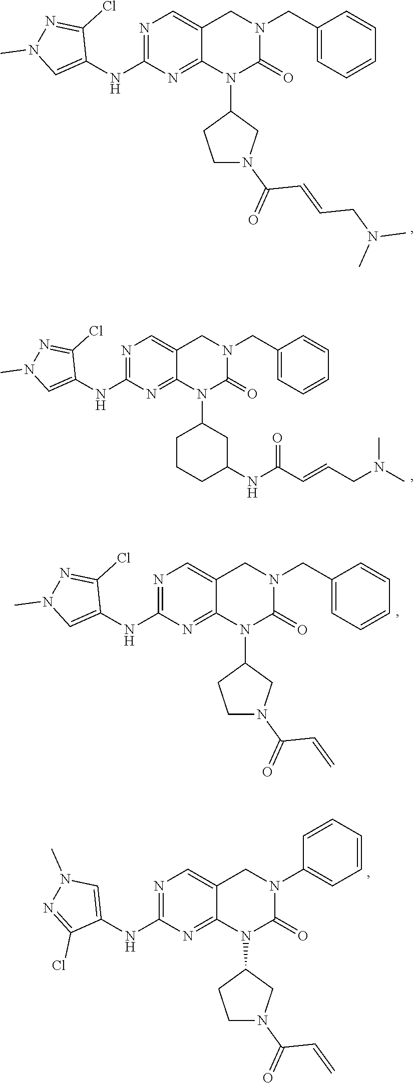

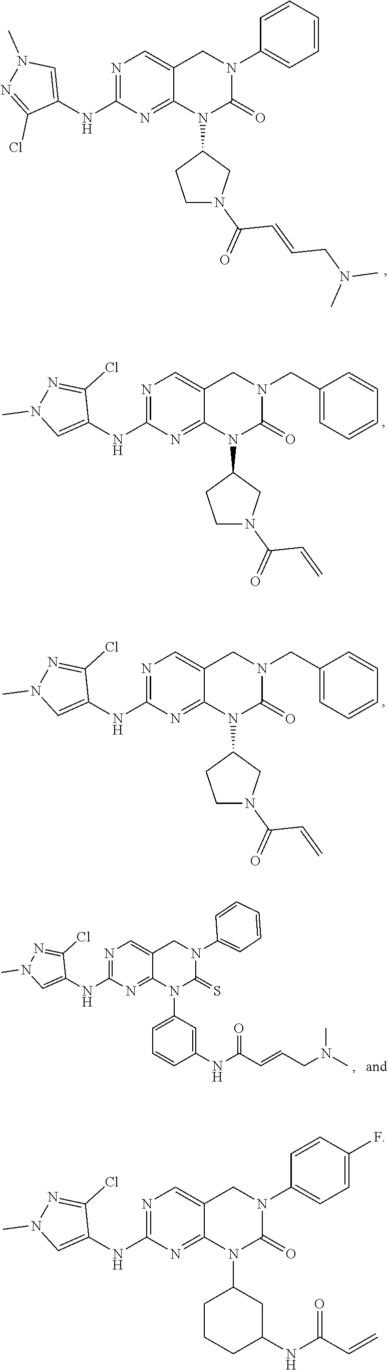

In another aspect, the present disclosure provides a pharmaceutical composition comprising a compound of Formula I, or a pharmaceutically acceptable salt thereof, and a pharmaceutically acceptable carrier.

Particular embodiments of the present disclosure are compounds of Formula I or its stereoisomers, tautomers, pharmaceutically acceptable salts, polymorphs, solvates and hydrates thereof, selected from the group consisting of,
N-(3-(3-benzyl-7-((1-methyl-1H-pyrazol-4-yl)amino)-2-oxo-3,4-dihydropyrimido[4,5-d]pyrimidin-1(2H)-yl)phenyl)acrylamide (Compound 1),
(E)-4-(dimethylamino)-N-(3-(3-(4-fluorobenzyl)-7-((1-methyl-1H-pyrazol-4-yl)amino)-2-oxo-3,4-dihydropyrimido[4,5-d]pyrimidin-1(2H)-yl)phenyl)but-2-enamide (Compound 2),
N-(3-(3-(4-fluorobenzyl)-7-((1-methyl-1H-pyrazol-4-yl)amino)-2-oxo-3,4-dihydropyrimido[4,5-d]pyrimidin-1(2H)-yl)phenyl)acrylamide (Compound 3),
N-(3-(3-benzyl-7-((1, 5-dimethyl-1H-pyrazol-4-yl)amino)-2-oxo-3,4-dihydropyrimido[4,5-d]pyrimidin-1(2H)-yl)phenyl)acrylamide (Compound 4),
N-(3-(3-benzyl-7-((1-cyclopropyl-1H-pyrazol-4-yl)amino)-2-oxo-3,4-dihydropyrimido[4,5-d]pyrimidin-1(2H)-yl)phenyl)acrylamide (Compound 5),
N-(3-(3-benzyl-7-((3-methyl-1-(2,2,2-trifluoroethyl)-1H-pyrazol-4-yl)amino)-2-oxo-3,4-dihydropyrimido[4,5-d]pyrimidin-1(2H)-yl)phenyl)acrylamide (Compound 6),
(E)-N-(3-(3-benzyl-7-((1-cyclopropyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 7),
(E)-N-(3-(3-benzyl-7-((3-methyl-1-(2,2,2-trifluoroethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 8),
(E)-N-(3-(3-benzyl-7-((1-isopropyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 9),
(E)-4-(dimethylamino)-N-(3-(7-((1-methyl-1H-pyrazol-4-yl)amino)-2-oxo-3-(1-phenylethyl)-3,4-dihydropyrimido[4,5-d]pyrimidin-1(2H)-yl)phenyl)but-2-enamide (Compound 10),
(E)-N-(3-(3-benzyl-7-((1,5-dimethyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 11),
(E)-N-(3-(3-benzyl-2-oxo-7-((1-(2,2,2-trifluoroethyl)-1H-pyrazol-4-yl)amino)-3,4-dihydropyrimido[4,5-d]pyrimidin-1(2H)-yl)phenyl)-4-(dimethylamino)but-2-enamide (Compound 12),
N-(3-(3-benzyl-7-((1-(2-hydroxyethyl)-1H-pyrazol-4-yl)amino)-2-oxo-3,4-dihydropyrimido[4,5-d]pyrimidin-1(2H)-yl)phenyl)acrylamide (Compound 13),
(E)-N-(3-(3-benzyl-7-((1-(2-hydroxyethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 14),
N-(3-(3-benzyl-7-((1-(2-methoxyethyl)-1H-pyrazol-4-yl)amino)-2-oxo-3,4-dihydropyrimido[4,5-d]pyrimidin-1(2H)-yl)phenyl)acrylamide (Compound 15),
(E)-N-(3-(3-benzyl-7-((1-(2-methoxyethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 16),
N-(3-(3-(4-fluorophenyl)-7-((1-methyl-1H-pyrazol-4-yl)amino)-2-oxo-3,4-dihydropyrimido[4,5-d]pyrimidin-1(2H)-yl)phenyl)acrylamide (Compound 17),
(E)-4-(dimethylamino)-N-(3-(3-(4-fluorophenyl)-7-((1-methyl-1H-pyrazol-4-yl)amino)-2-oxo-3,4-dihydropyrimido[4,5-d]pyrimidin-1(2H)-yl)phenyl)but-2-enamide (Compound 18),
(E)-N-(3-(3-benzyl-7-((5-methylthiazol-2-yl)amino)-2-oxo-3,4-dihydropyrimido[4,5-d]pyrimidin-1(2H)-yl)phenyl)-4-(dimethylamino)but-2-enamide (Compound 19),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 20),
(E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-4-(dimethylamino)-N-methylbut-2-enamide (Compound 21),
(E)-N-(3-(3-benzyl-7-((3-methylisothiazol-5-yl)amino)-2-oxo-3,4-dihydropyrimido[4,5-d]pyrimidin-1(2H)-yl)phenyl)-4-(dimethylamino)but-2-enamide (Compound 22),
(E)-N-(3-(3-benzyl-7-((1,3-dimethyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 23),
N-(3-(3-benzyl-7-((1-methyl-1H-pyrazol-4-yl)amino)-2-oxo-3,4-dihydropyrimido[4,5-d]pyrimidin-1(2H)-yl)cyclohexyl)acrylamide (Compound 24),
N-(3-(3-benzyl-7-((4-methylthiazol-2-yl)amino)-2-oxo-3,4-dihydropyrimido[4,5-d]pyrimidin-1(2H)-yl)phenyl)acrylamide (Compound 25),
(E)-N-(3-(3-benzyl-7-((1-methyl-1H-1,2,3-triazol-4-yl)amino)-2-oxo-3,4-dihydropyrimido[4,5-d]pyrimidin-1(2H)-yl)phenyl)-4-(dimethylamino)but-2-enamide (Compound 26),
(E)-N-(3-(3-benzyl-7-((5-methylthiazol-2-yl)amino)-2-oxo-3,4-dihydropyrimido[4,5-d]pyrimidin-1(2H)-yl)cyclohexyl)-4-(dimethylamino)but-2-enamide (Compound 27),
N-(3-(3-benzyl-7-((3-methylisothiazol-5-yl)amino)-2-oxo-3,4-dihydropyrimido[4,5-d]pyrimidin-1(2H)-yl)cyclohexyl)acrylamide (Compound 28),
(E)-N-(3-(3-benzyl-7-((4-methylthiazol-2-yl)amino)-2-oxo-3,4-dihydropyrimido[4,5-d]pyrimidin-1(2H)-yl)cyclohexyl)-4-(dimethylamino)but-2-enamide (Compound 29),
N-(3-(3-benzyl-7-((5-methylthiazol-2-yl)amino)-2-oxo-3,4-dihydropyrimido[4,5-d]pyrimidin-1(2H)-yl)cyclohexyl)acrylamide (Compound 30),
(E)-N-(3-(3-benzyl-7-((1-methyl-1H-pyrazol-4-yl)amino)-2-oxo-3,4-dihydropyrimido[4,5-d]pyrimidin-1(2H)-yl)cyclohexyl)-4-(dimethylamino)but-2-enamide (Compound 31),
(E)-N-(3-(3-benzyl-7-((3-methylisothiazol-5-yl)amino)-2-oxo-3,4-dihydropyrimido[4,5-d]pyrimidin-1(2H)-yl)cyclohexyl)-4-(dimethylamino)but-2-enamide (Compound 32),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)cyclohexyl)-4-(dimethylamino)but-2-enamide (Compound 33),
(E)-3-benzyl-7-((3-chloro-1-methyl-1H-pyrazol-4-yl)amino)-1-(1-(4-(dimethylamino)but-2-enoyl)pyrrolidin-3-yl)-3,4-dihydropyrimido[4,5-d]pyrimidin-2(1H)-one (Compound 34),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(dimethylamino)but-2-enamide (Compound 35),
N-(3-(7-((3-chloro-1-methyl-1H-pyrazol-4-yl)amino)-3-(4-fluorophenyl)-2-oxo-3,4-dihydropyrimido[4,5-d]pyrimidin-1(2H)-yl)cyclohexyl)acrylamide (Compound 36),
1-(1-acryloylpyrrolidin-3-yl)-3-benzyl-7-((3-chloro-1-methyl-1H-pyrazol-4-yl)amino)-3,4-dihydropyrimido[4,5-d]pyrimidin-2(1H)-one (Compound 37), (Racemic),
(R)-1-(1-acryloylpyrrolidin-3-yl)-3-benzyl-7-((3-chloro-1-methyl-1H-pyrazol-4-yl)amino)-3,4-dihydropyrimido[4,5-d]pyrimidin-2(1H)-one(Compound 38), (Enantiomer 1),
(S)-1-(1-acryloylpyrrolidin-3-yl)-3-benzyl-7-((3-chloro-1-methyl-1H-pyrazol-4-yl)amino)-3,4-dihydropyrimido[4,5-d]pyrimidin-2(1H)-one (Compound 39), (Enantiomer 2),
N-(3-(3-benzyl-7-((3-chloro-1-methyl-1H-pyrazol-4-yl)amino)-2-oxo-3,4-dihydropyrimido[4,5-d]pyrimidin-1(2H)-yl)cyclohexyl)acrylamide (Compound 40),
(S)-1-(1-acryloylpyrrolidin-3-yl)-7-((3-chloro-1-methyl-1H-pyrazol-4-yl)amino)-3-phenyl-3,4-dihydropyrimido[4,5-d]pyrimidin-2(1H)-one (Compound 41),
(S,E)-7-((3-chloro-1-methyl-1H-pyrazol-4-yl)amino)-1-(1-(4-(dimethylamino)but-2-enoyl)pyrrolidin-3-yl)-3-phenyl-3,4-dihydropyrimido[4,5-d]pyrimidin-2(1H)-one (Compound 42),
(E)-N-(3-(3-benzyl-7-((1-methyl-1H-indazol-5-yl)amino)-2-oxo-3,4-dihydropyrimido[4,5-d]pyrimidin-1(2H)-yl)phenyl)-4-(dimethylamino)but-2-enamide (Compound 43),
(E)-N-(3-(3-benzyl-7-((2-cyano-1-methyl-1H-indol-5-yl)amino)-2-oxo-3,4-dihydropyrimido[4,5-d]pyrimidin-1(2H)-yl)phenyl)-4-(dimethylamino)but-2-enamide (Compound 44),
(E)-N-(3-(3-benzyl-7-((1-methyl-1H-indazol-6-yl)amino)-2-oxo-3,4-dihydropyrimido[4,5-d]pyrimidin-1(2H)-yl)phenyl)-4-(dimethylamino)but-2-enamide (Compound 45),
N-(3-(3-benzyl-7-((6-morpholinopyridin-3-yl)amino)-2-oxo-3,4-dihydropyrimido[4,5-d]pyrimidin-1(2H)-yl)phenyl)acrylamide (Compound 46),
N-(3-(3-benzyl-7-((1-methyl-1H-pyrazol-4-yl)amino)-2-oxo-3,4-dihydropyrimido[4,5-d]pyrimidin-1(2H)-yl)phenyl)ethenesulfonamide (Compound 47),
(E)-N-(3-(3-benzyl-7-((4-methylthiazol-2-yl)amino)-2-oxo-3,4-dihydropyrimido[4,5-d]pyrimidin-1(2H)-yl)phenyl)-4-(dimethylamino)but-2-enamide (Compound 48),
(E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 49),
(E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-3-chloroacrylamide (Compound 50),
(E)-N-(3-(7-((3-chloro-1-methyl-1H-pyrazol-4-yl)amino)-3-phenyl-2-thioxo-3,4-dihydropyrimido[4,5-d]pyrimidin-1(2H)-yl)phenyl)-4-(dimethylamino)but-2-enamide (Compound 51),
(E)-N-(3-(3-benzyl-7-((1-methyl-1H-pyrazol-3-yl)amino)-2-oxo-3,4-dihydropyrimido[4,5-d]pyrimidin-1(2H)-yl)phenyl)-4-(dimethylamino)but-2-enamide (Compound 52),
(E)-N-(3-(3-benzyl-7-((1-methyl-1H-pyrazolo[3,4-b]pyridin-5-yl)amino)-2-oxo-3,4-dihydropyrimido[4,5-d]pyrimidin-1(2H)-yl)phenyl)-4-(dimethylamino)but-2-enamide (Compound 53),
(E)-N-(3-(3-benzyl-7-((6-morpholinopyridin-3-yl)amino)-2-oxo-3,4-dihydropyrimido[4,5-d]pyrimidin-1(2H)-yl)phenyl)-4-(dimethylamino)but-2-enamide (Compound 54),
(E)-N-(3-(3-benzyl-7-((2-morpholinopyrimidin-5-yl)amino)-2-oxo-3,4-dihydropyrimido[4,5-d]pyrimidin-1(2H)-yl)phenyl)-4-(dimethylamino)but-2-enamide (Compound 55),
(E)-N-(3-(3-benzyl-7-((3-methoxy-1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 56),
(E)-N-(3-(7-((3-chloro-1-methyl-1H-pyrazol-4-yl)amino)-3-(4-fluorobenzyl)-2-oxo-3,4-dihydropyrimido[4,5-d]pyrimidin-1(2H)-yl)phenyl)-4-(dimethylamino)but-2-enamide (Compound 57),
(E)-N-(3-(3-benzyl-7-(isoxazol-4-ylamino)-2-oxo-3,4-dihydropyrimido[4,5-d]pyrimidin-1(2H)-yl)phenyl)-4-(dimethylamino)but-2-enamide (Compound 58),
(E)-N-(3-(3-benzyl-7-((2-(4-methylpiperazin-1-yl)pyrimidin-5-yl)amino)-2-oxo-3,4-dihydropyrimido[4,5-d]pyrimidin-1(2H)-yl)phenyl)-4-(dimethylamino)but-2-enamide (Compound 59),
(E)-N-(3-(3-benzyl-7-((3-methylisoxazol-4-yl)amino)-2-oxo-3,4-dihydropyrimido[4,5-d]pyrimidin-1(2H)-yl)phenyl)-4-(dimethylamino)but-2-enamide (Compound 60),
(E)-N-(3-(7-((3-chloro-1-methyl-1H-pyrazol-4-yl)amino)-3-(2,4-difluorobenzyl)-2-oxo-3,4-dihydropyrimido[4,5-d]pyrimidin-1(2H)-yl)phenyl)-4-(dimethylamino)but-2-enamide (Compound 61),
(E)-N-(3-(7-((3-chloro-1-methyl-1H-pyrazol-4-yl)amino)-3-((2-methoxypyridin-4-yl)methyl)-2-oxo-3,4-dihydropyrimido[4,5-d]pyrimidin-1(2H)-yl)phenyl)-4-(dimethylamino)but-2-enamide (Compound 62),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)-4-(dimethylamino)-N-methylbut-2-enamide (Compound 63),
(E)-N-(3-(3-benzyl-7-((3-chloro-1-methyl-1H-pyrazol-4-yl)amino)-2-oxo-3,4-dihydropyrido[4,3-d]pyrimidin-1(2H)-yl)phenyl)-4-(dimethylamino)but-2-enamide (Compound 64),
(E)-N-(3-(3-benzyl-7-((3-chloro-1-(1-methylpiperidin-4-yl)-1H-pyrazol-4-yl)amino)-2-oxo-3,4-dihydropyrimido[4,5-d]pyrimidin-1(2H)-yl)phenyl)-4-(dimethylamino)but-2-enamide trifluoroacetate salt (Compound 65),
(E)-N-(3-(3-benzyl-7-((6-(4-methylpiperazin-1-yl)pyridin-3-yl)amino)-2-oxo-3,4-dihydropyrimido[4,5-d]pyrimidin-1(2H)-yl)phenyl)-4-(dimethylamino)but-2-enamide (Compound 66),
N-(3-(3-benzyl-7-((6-(4-methylpiperazin-1-yl)pyridin-3-yl)amino)-2-oxo-3,4-dihydropyrimido[4,5-d]pyrimidin-1(2H)-yl)phenyl)acrylamide (Compound 67),
(E)-N-(3-(7-((3-chloro-1-methyl-1H-pyrazol-4-yl)amino)-3-(2,4-dimethylbenzyl)-2-oxo-3,4-dihydropyrimido[4,5-d]pyrimidin-1(2H)-yl)phenyl)-4-(dimethylamino)but-2-enamide (Compound 68),
N-(3-(7-((3-chloro-1-methyl-1H-pyrazol-4-yl)amino)-3-(2,4-dimethylbenzyl)-2-oxo-3,4-dihydropyrimido[4,5-d]pyrimidin-1(2H)-yl)phenyl)acrylamide (Compound 69),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)-4-(piperidin-1-yl)but-2-enamide (Compound 70),
N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)acrylamide (Compound 71),
N-(3-(3-benzyl-7-((6-(4-(dimethylamino)piperidin-1-yl)pyridin-3-yl)amino)-2-oxo-3,4-dihydropyrimido[4,5-d]pyrimidin-1(2H)-yl)phenyl)acrylamide (Compound 72),
N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)acrylamide (Compound 73),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)-4-fluorobut-2-enamide (Compound 74),
(E)-N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 75),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)phenyl)-4-methoxybut-2-enamide (Compound 76),
N-(5-(3-benzyl-7-((3-chloro-1-methyl-1H-pyrazol-4-yl)amino)-2-oxo-3,4-dihydropyrimido[4,5-d]pyrimidin-1(2H)-yl)-2-methoxyphenyl)acrylamide (Compound 77),
N-(5-(3-benzyl-7-((3-chloro-1-methyl-1H-pyrazol-4-yl)amino)-2-oxo-3,4-dihydropyrimido[4,5-d]pyrimidin-1(2H)-yl)-2-(trifluoromethoxy)phenyl)acrylamide (Compound 78),
N-(3-(3-benzyl-7-((1-methylindolin-4-yl)amino)-2-oxo-3,4-dihydropyrimido[4,5-d]pyrimidin-1(2H)-yl)phenyl)acrylamide (Compound 79), (E)-N-(3-(3-benzyl-7-((1-methylindolin-4-yl)amino)-2-oxo-3,4-dihydropyrimido[4,5-d]pyrimidin-1(2H)-yl)phenyl)-4-(dimethylamino)but-2-enamide (Compound 80),
N-(3-(3-benzyl-7-((3-chloro-1-methyl-1H-pyrazol-4-yl)amino)-2-oxo-3,4-dihydropyrimido[4,5-d]pyrimidin-1(2H)-yl)-4-fluorophenyl)acrylamide (Compound 81),
(E)-N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)-4-(piperidin-1-yl)but-2-enamide (Compound 82),
(E)-N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)-4-methoxybut-2-enamide (Compound 83),
N-(3-(7-((3-chloro-1-methyl-1H-pyrazol-4-yl)amino)-3-((6-fluoropyridin-3-yl)methyl)-2-oxo-3,4-dihydropyrimido[4,5-d]pyrimidin-1(2H)-yl)phenyl)acrylamide (Compound 84),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)-4-fluorophenyl)-4-(dimethylamino)but-2-enamide (Compound 85),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)-4-chlorophenyl)-4-(dimethylamino)but-2-enamide (Compound 86),
(E)-N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)but-2-enamide (Compound 87),
(E)-N-(3-(3-benzyl-7-((3-fluoro-1-methyl-1H-pyrazol-4-yl)amino)-2-oxo-3,4-dihydropyrimido[4,5-d]pyrimidin-1(2H)-yl)phenyl)-4-fluorobut-2-enamide (Compound 88),
(E)-N-(3-(3-benzyl-7-((3-chloro-1-methyl-1H-pyrazol-4-yl)amino)-2-oxo-3,4-dihydropyrimido[4,5-d]pyrimidin-1(2H)-yl)-4-fluorophenyl)-4-fluorobut-2-enamide (Compound 89),
(E)-N-(3-(3-benzyl-2-oxo-7-(pyrazolo[1,5-a]pyrimidin-3-ylamino)-3,4-dihydropyrimido[4,5-d]pyrimidin-1(2H)-yl)phenyl)-4-(dimethylamino)but-2-enamide (Compound 90),
(E)-N-(3-(3-benzyl-2-oxo-7-(pyrazolo[1,5-a]pyridin-3-ylamino)-3,4-dihydropyrimido[4,5-d]pyrimidin-1(2H)-yl)phenyl)-4-(dimethylamino)but-2-enamide (Compound 91),
(E)-N-(3-(3-benzyl-7-((3-chloro-1-(2-methoxyethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 92),
(E)-N-(3-(3-benzyl-7-((3-chloro-1-(2-fluoroethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 93),
(E)-N-(3-(3-benzyl-7-((3-(difluoromethyl)-1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 94),
(E)-N-(3-(3-benzyl-7-((1-methyl-3-(trifluoromethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 95),
(E)-N-(3-(3-benzyl-2-oxo-7-(pyridin-4-ylamino)-3,4-dihydropyrimido[4,5-d]pyrimidin-1(2H)-yl)phenyl)-4-(dimethylamino)but-2-enamide (Compound 96),
(E)-N-(3-(3-benzyl-7-((6,7-dihydro-5H-pyrazolo[5,1-b][1,3]oxazin-3-yl)amino)-2-oxo-3,4-dihydropyrimido[4,5-d]pyrimidin-1(2H)-yl)phenyl)-4-(dimethylamino)but-2-enamide (Compound 97),
(E)-N-(3-(3-benzyl-7-((1-(1-methylpiperidin-4-yl)indolin-4-yl)amino)-2-oxo-3,4-dihydropyrimido[4,5-d]pyrimidin-1(2H)-yl)phenyl)-4-(dimethylamino)but-2-enamide (Compound 98),
(E)-N-(3-(3-benzyl-7-((1-methyl-1H-indol-5-yl)amino)-2-oxo-3,4-dihydropyrimido[4,5-d]pyrimidin-1(2H)-yl)phenyl)-4-(dimethylamino)but-2-enamide (Compound 99),
(E)-N-(3-(3-benzyl-7-((2-methylimidazo[1,2-a]pyridin-6-yl)amino)-2-oxo-3,4-dihydropyrimido[4,5-d]pyrimidin-1(2H)-yl)phenyl)-4-(dimethylamino)but-2-enamide (Compound 100),
(E)-N-(3-(3-benzyl-7-((3-chloro-1-(2,2-difluoroethyl)-1H-pyrazol-4-yl)amino)-2-oxo-3,4-dihydropyrimido[4,5-d]pyrimidin-1(2H)-yl)phenyl)-4-(dimethylamino)but-2-enamide (Compound 101),
(E)-N-(3-(3-benzyl-7-((1-methyl-1H-benzo[d]imidazol-5-yl)amino)-2-oxo-3,4-dihydropyrimido[4,5-d]pyrimidin-1(2H)-yl)phenyl)-4-(dimethylamino)but-2-enamide (Compound 102),
(E)-N-(3-(3-benzyl-7-((2-methoxypyridin-3-yl)amino)-2-oxo-3,4-dihydropyrimido[4,5-d]pyrimidin-1(2H)-yl)phenyl)-4-(dimethylamino)but-2-enamide trifluoroacetate salt (Compound 103),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(dimethylamino)-N-methylbut-2-enamide (Compound 104),
N-(3-(7-((3-chloro-1-(1-methylpiperidin-4-yl)-1H-pyrazol-4-yl)amino)-2-oxo-3-phenyl-3,4-dihydropyrimido[4,5-d]pyrimidin-1(2H)-yl)phenyl)acrylamide (Compound 105),
(E)-N-(3-(7-((3-chloro-1-(1-methylpiperidin-4-yl)-1H-pyrazol-4-yl)amino)-2-oxo-3-phenyl-3,4-dihydropyrimido[4,5-d]pyrimidin-1(2H)-yl)phenyl)-4-(dimethylamino)but-2-enamide (Compound 106),
(E)-4-(dimethylamino)-N-(3-(7-((1-(4-hydroxycyclohexyl)-1H-pyrazol-4-yl)amino)-2-oxo-3-phenyl-3,4-dihydropyrimido[4,5-d]pyrimidin-1(2H)-yl)phenyl)but-2-enamide (Compound 107),
N-(3-(7-((1-(4-hydroxycyclohexyl)-1H-pyrazol-4-yl)amino)-2-oxo-3-phenyl-3,4-dihydropyrimido[4,5-d]pyrimidin-1(2H)-yl)phenyl)acrylamide (Compound 108),
(E)-4-(dimethylamino)-N-(3-(7-((6-(4-methylpiperazin-1-yl)pyridin-3-yl)amino)-2-oxo-3-phenyl-3,4-dihydropyrimido[4,5-d]pyrimidin-1(2H)-yl)phenyl)but-2-enamide (Compound 109),
N-(3-(7-((6-(4-methylpiperazin-1-yl)pyridin-3-yl)amino)-2-oxo-3-phenyl-3,4-dihydropyrimido[4,5-d]pyrimidin-1(2H)-yl)phenyl)acrylamide (Compound 110),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(piperidin-1-yl)but-2-enamide (Compound 111),
N-(3-(7-((6-(4-(dimethylamino)piperidin-1-yl)pyridin-3-yl)amino)-2-oxo-3-phenyl-3,4-dihydropyrimido[4,5-d]pyrimidin-1(2H)-yl)phenyl)acrylamide (Compound 112),
N-(3-(7-((3-fluoro-1-methyl-1H-pyrazol-4-yl)amino)-2-oxo-3-phenyl-3,4-dihydropyrimido[4,5-d]pyrimidin-1(2H)-yl)phenyl)acrylamide (Compound 113),
(E)-4-(dimethylamino)-N-(3-(7-((2-fluoropyridin-3-yl)amino)-2-oxo-3-phenyl-3,4-dihydropyrimido[4,5-d]pyrimidin-1(2H)-yl)phenyl)but-2-enamide (Compound 114),
N-(3-(7-((2-fluoropyridin-3-yl)amino)-2-oxo-3-phenyl-3,4-dihydropyrimido[4,5-d]pyrimidin-1(2H)-yl)phenyl)acrylamide (Compound 115),
N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)acrylamide (Compound 116),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-fluorobut-2-enamide (Compound 117),
N-(3-(7-((1-(1-methylpiperidin-4-yl)indolin-4-yl)amino)-2-oxo-3-phenyl-3,4-dihydropyrimido[4,5-d]pyrimidin-1(2H)-yl)phenyl)acrylamide (Compound 118),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-methoxybut-2-enamide (Compound 119),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)but-2-enamide (Compound 120),
N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)methacrylamide (Compound 121),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-morpholinobut-2-enamide (Compound 122),
N-(3-(7-((3-chloro-1-(2-cyanopropan-2-yl)-1H-pyrazol-4-yl)amino)-2-oxo-3-phenyl-3,4-dihydropyrimido[4,5-d]pyrimidin-1(2H)-yl)phenyl)acrylamide trifluoroacetate salt (Compound 123),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(4-(dimethylamino)piperidin-1-yl)but-2-enamide (Compound 124),
N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)but-2-ynamide (Compound 125),
(E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(4-methylpiperazin-1-yl)but-2-enamide (Compound 126),
N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-2-methoxyacrylamide (Compound 127),
N-(3-(7-((3-chloro-1-methyl-1H-pyrazol-4-yl)amino)-2-oxo-3-phenyl-3,4-dihydropyrimido[4,5-d]pyrimidin-1(2H)-yl)-4-fluorophenyl)acrylamide trifluoroacetate salt (Compound 128),
N-(3-(7-((3-chloro-1-(4-hydroxycyclohexyl)-1H-pyrazol-4-yl)amino)-2-oxo-3-phenyl-3,4-dihydropyrimido[4,5-d]pyrimidin-1(2H)-yl)phenyl)acrylamide trifluoroacetate salt (Compound 129),
N-(2-(7-((3-chloro-1-methyl-1H-pyrazol-4-yl)amino)-2-oxo-3-phenyl-3,4-dihydropyrimido[4,5-d]pyrimidin-1(2H)-yl)phenyl)acrylamide (Compound 130),
N-(4-(7-((3-chloro-1-methyl-1H-pyrazol-4-yl)amino)-2-oxo-3-phenyl-3,4-dihydropyrimido[4,5-d]pyrimidin-1(2H)-yl)phenyl)acrylamide (Compound 131),
N-(3-(7-((3-chloro-1-(piperidin-4-yl)-1H-pyrazol-4-yl)amino)-2-oxo-3-phenyl-3,4-dihydropyrimido[4,5-d]pyrimidin-1(2H)-yl)phenyl)acrylamide trifluoroacetate salt (Compound 132),
(R,E)-4-(dimethylamino)-N-(3-(7-((1-methyl-1H-pyrazol-4-yl)amino)-2-oxo-3-(1-phenylethyl)-3,4-dihydropyrimido[4,5-d]pyrimidin-1(2H)-yl)phenyl)but-2-enamide (Compound 133),
(S,E)-4-(dimethylamino)-N-(3-(7-((1-methyl-1H-pyrazol-4-yl)amino)-2-oxo-3-(1-phenylethyl)-3,4-dihydropyrimido[4,5-d]pyrimidin-1(2H)-yl)phenyl)but-2-enamide (Compound 134),
(E)-4-fluoro-N-(3-(7-((2-fluoropyridin-3-yl)amino)-2-oxo-3-phenyl-3,4-dihydropyrimido[4,5-d]pyrimidin-1(2H)-yl)phenyl)but-2-enamide (Compound 135),
N-(3-(7-((3-chloro-1-(oxetan-3-yl)-1H-pyrazol-4-yl)amino)-2-oxo-3-phenyl-3,4-dihydropyrimido[4,5-d]pyrimidin-1(2H)-yl)phenyl)acrylamide (Compound 136).

An embodiment of the present disclosure relates to a compound of Formula I or its stereoisomers, tautomers, pharmaceutically acceptable salts, polymorphs, solvates and hydrates thereof, for treating disease associated with epidermal growth factor receptor (EGFR) family kinases.

Another embodiment of the present disclosure relates to a compound of Formula I or its stereoisomers, tautomers, pharmaceutically acceptable salts, polymorphs, solvates and hydrates thereof, for treating cancer.

Another embodiment of the present disclosure relates to a compound Formula I, or its stereoisomers, tautomers, pharmaceutically acceptable salts, polymorphs, solvates and hydrates thereof, for treating disease or condition associated with non-small cell or small cell lung cancer or prostate cancer or head and neck cancer or breast cancer or colorectal cancer.

The present disclosure relates to a pharmaceutical composition comprising a compound of Formula I or a pharmaceutically acceptable salt thereof together with a pharmaceutically acceptable carrier, optionally in combination with one or more other pharmaceutical compositions.

The present disclosure further relates to the process of preparation of compounds of Formula I or its stereoisomers, tautomers, pharmaceutically acceptable salts, polymorphs, solvates and hydrates thereof.

### Uses

Some embodiments provided herein describe a class of compounds that are useful as epidermal growth factor receptor (EGFR) family kinase inhibitors. Some embodiments provided herein describe a class of compounds that are useful as HER2 inhibitors. Some embodiments provided herein describe a class of compounds that are useful as EGFR inhibitors. Some embodiments provided herein describe a class of compounds that are useful as EGFR del19/T790M inhibitors. Some embodiments provided herein describe a class of compounds that are useful as EGFR L858R/T790M inhibitors. In some embodiments, the compounds described herein have improved potency and/or beneficial activity profiles and/or beneficial selectivity profiles and/or increased efficacy and/or improved safety profiles (such as reduced side effects) and/or improved pharmacokinetic properties. In some embodiments, the compounds described herein are selective inhibitors of EGFR del19/T790M over WT EGFR. In some embodiments, the compounds described herein are selective inhibitors of EGFR L858R/T790M over WT EGFR.

In some embodiments, the compounds described herein are useful to treat, prevent or ameliorate a disease or condition which displays drug resistance associated with EGFR del19/T790M activation. In some embodiments, the compounds described herein are useful to treat, prevent or ameliorate a disease or condition which displays drug resistance associated with EGFR L858R/T790M activation.

In some embodiments, EGFR family kinase mutants are detected with a commercially available test kit. In some embodiments, EGFR family kinase mutants are detected with a reverse transcription polymerase chain reaction (RT-PCR)-based method. In some embodiments, EGFR family kinase mutants are detected with a sequencing-based method. In some embodiments, EGFR family kinase mutants are detected with a mass spectrometry genotyping-based method. In some embodiments, EGFR family kinase mutants are detected with an immunohistochemistry-based method. In some embodiments, EGFR family kinase mutants are detected with a molecular diagnostics panel. In some embodiments, EGFR family kinase mutants are detected from a tumor sample. In some embodiments, EGFR family kinase mutants are detected from circulating DNA. In some embodiments, EGFR family kinase mutants are detected from tumor cells.

In one aspect, provided herein is a method of inhibiting an epidermal growth factor receptor (EGFR) family kinase mutant in a subject in need thereof, comprising administering to the subject a therapeutically effective amount of a compound of Formula I, or a pharmaceutically acceptable salt thereof.

In another aspect, provided herein is a method of inhibiting a human epidermal growth factor receptor 2 (HER2) mutant in a subject in need thereof, comprising administering to the subject a therapeutically effective amount of a compound of Formula I, or a pharmaceutically acceptable salt thereof. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutant is A775ins_G776insYVMA. In some embodiments, the HER2 mutant is A775_G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780 Y781insGSP.

In another aspect, provided herein is a method of inhibiting an epidermal growth factor receptor (EGFR) mutant in a subject in need thereof, comprising administering to the subject a therapeutically effective amount of a compound of Formula I, or a pharmaceutically acceptable salt thereof.

In another aspect, provided herein is a method of inhibiting a drug-resistant epidermal growth factor receptor (EGFR) mutant in a subject in need thereof, comprising administering to the subject a therapeutically effective amount of a compound of Formula I, or a pharmaceutically acceptable salt thereof. In some embodiments, the drug-resistant EGFR mutant is del19/T790M EGFR or L858R/T790M EGFR.

In another aspect, provided herein is a method of inhibiting human epidermal growth factor receptor 2 (HER2) in a subject in need thereof, comprising administering to the subject a therapeutically effective amount of a compound of Formula I, or a pharmaceutically acceptable salt thereof, wherein the compound exhibits greater inhibition of a HER2 mutant relative to wild-type EGFR. In some embodiments, the HER2 mutant comprises an insertion in exon 20, an in-frame deletion and insertion in exon 20, a substitution in the extracellular domain, an extracellular truncation, or a substitution in exon 30. In some embodiments, the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780 Y781insGSP. In some embodiments, the HER2 mutant is A775ins_G776insYVMA. In some embodiments, the HER2 mutant is A775_G776insSVMA. In some embodiments, the HER2 mutant is A775_G776insVVMA. In some embodiments, the HER2 mutant is G776del insVC. In some embodiments, the HER2 mutant is G776del insLC. In some embodiments, the HER2 mutant is G776del insAV. In some embodiments, the HER2 mutant is G776del insAVGC. In some embodiments, the HER2 mutant is S310F. In some embodiments, the HER2 mutant is S310Y. In some embodiments, the HER2 mutant is p95. In some embodiments, the HER2 mutant is V842I. In some embodiments, the HER2 mutant is P780_Y781insGSP.

In another aspect, provided herein is a method of inhibiting epidermal growth factor receptor (EGFR) in a subject in need thereof, comprising administering to the subject a therapeutically effective amount of a compound of Formula I, or a pharmaceutically acceptable salt thereof, wherein the compound exhibits greater inhibition of an EGFR mutant relative to wild-type EGFR.

In some embodiments, the EGFR mutant comprises a substitution in exon 18, a deletion in exon 19, a substitution in exon 20, an insertion in exon 20, a mutation in the extracellular domain, or a substitution in exon 21. In some embodiments, the EGFR mutant is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutant is del19/T790M EGFR or L858R/T790M EGFR. In some embodiments, the EGFR mutant is del19/T790M EGFR. In some embodiments, the EGFR mutant is L858R/T790M EGFR.

In another aspect, provided herein is a method of treating a disease or disorder associated with epidermal growth factor receptor (EGFR) in a subject in need thereof, comprising administering to the subject a therapeutically effective amount of a compound of Formula I, or a pharmaceutically acceptable salt thereof.

In some embodiments, the disease or disorder in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutation is A775ins_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the disease or disorder in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

In another aspect, provided herein is a method of treating cancer in a subject in need thereof, comprising administering to the subject a therapeutically effective amount of a compound of Formula I, or a pharmaceutically acceptable salt thereof. In some embodiments, the cancer displays drug resistance associated with EGFR del19/T790M activation. In some embodiments, the cancer displays drug resistance associated with EGFR L858R/T790M activation. Other embodiments provided herein describe the use of the compounds described herein for treating cancer.

In some embodiments, the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer. In some embodiments, the cancer is selected from non-small cell lung cancer, prostate cancer, head and neck cancer, breast cancer, colorectal cancer, and glioblastoma. In some embodiments, the cancer is non-small cell lung cancer. In some embodiments, the cancer is prostate cancer. In some embodiments, the cancer is head and neck cancer. In some embodiments, the cancer is breast cancer. In some embodiments, the cancer is colorectal cancer. In some embodiments, the cancer is glioblastoma.

In some embodiments, the cancer in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775 G776insSVMA, A775_G776insVVMA, G776del insVC, G776del insLC, G776del insAV, G776del insAVGC, S310F, S310Y, p95, V842I, or P780_Y781insGSP. In some embodiments, the HER2 mutation is A775ins_G776insYVMA. In some embodiments, the HER2 mutation is A775_G776insSVMA. In some embodiments, the HER2 mutation is A775 G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780_Y781insGSP.

In some embodiments, the cancer in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

In another aspect, provided herein is a method of treating inflammatory disease in a subject in need thereof, comprising administering to the subject a therapeutically effective amount of a compound of Formula I, or a pharmaceutically acceptable salt thereof. Also described herein is the use of the compounds described herein for treating inflammatory diseases associated with EGFR del19/T790M activation. Also described herein is the use of the compounds described herein for treating inflammatory diseases associated with EGFR L858R/T790M activation.

In some embodiments, the inflammatory disease is selected from psoriasis, eczema, and atherosclerosis. In some embodiments, the inflammatory disease is psoriasis. In some embodiments, the inflammatory disease is eczema. In some embodiments, the inflammatory disease is atherosclerosis.

In some embodiments, the inflammatory disease in the subject comprises a HER2 mutation. In some embodiments, the HER2 mutation comprises an insertion in exon 20, an in-frame deletion and insertion in exon 20, a substitution in the extracellular domain, an extracellular truncation, or a substitution in exon 30. In some embodiments, the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780 Y781insGSP. In some embodiments, the HER2 mutation is A775ins_G776insYVMA. In some embodiments, the HER2 mutation is A775_G776insSVMA. In some embodiments, the HER2 mutation is A775_G776insVVMA. In some embodiments, the HER2 mutation is G776del insVC. In some embodiments, the HER2 mutation is G776del insLC. In some embodiments, the HER2 mutation is G776del insAV. In some embodiments, the HER2 mutation is G776del insAVGC. In some embodiments, the HER2 mutation is S310F. In some embodiments, the HER2 mutation is S310Y. In some embodiments, the HER2 mutation is p95. In some embodiments, the HER2 mutation is V842I. In some embodiments, the HER2 mutation is P780 Y781insGSP.

In some embodiments, the inflammatory disease in the subject comprises an EGFR mutation. In some embodiments, the EGFR mutation comprises a substitution in exon 18, a deletion in exon 19, a substitution in exon 20, an insertion in exon 20, a mutation in the extracellular domain, or a substitution in exon 21. In some embodiments, the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR. In some embodiments, the EGFR mutation is del19/T790M EGFR or L858R/T790M EGFR. In some embodiments, the EGFR mutation is del19/T790M EGFR. In some embodiments, the EGFR mutation is L858R/T790M EGFR.

### Administration and Pharmaceutical Composition

In certain embodiments, the EGFR inhibitory compound as described herein is administered as a pure chemical. In other embodiments, the EGFR inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, Pa. (2005)).

Provided herein is a pharmaceutical composition comprising at least one EGFR inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising a compound disclosed herein, or a pharmaceutically acceptable salt, solvate, or prodrug thereof, and a pharmaceutically acceptable excipient.

In certain embodiments, the EGFR inhibitory compound disclosed herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, Pa. (2005)).

The dose of the composition comprising at least one EGFR inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### EXAMPLES

### Example 1: Synthetic Procedures

Yields reported herein refer to purified products (unless specified) and are not optimised. Analytical TLC was performed on Merck silica gel 60 F₂₅₄ aluminum-backed plates. Compounds were visualised by UV light and/or stained either with iodine, potassium permanganate or ninhydrin solution. Flash column chromatography was performed on silica gel (100-200 M) or flash chromatography. ¹H-NMR spectra were recorded on a Bruker Avance-400 MHz spectrometer with a BBO (Broad Band Observe) and BBFO (Broad Band Fluorine Observe) probe. Chemical shifts (δ) are expressed in parts per million (ppm) downfield by reference to tetramethylsilane (TMS) as the internal standard. Splitting patterns are designated as s (singlet), d (doublet), t (triplet), q (quartet), m (multiplet) and bs (broad singlet). Coupling constants (J) are given in hertz (Hz). LC-MS analyses were performed on either an Acquity BEH C-18 column (2.10×100 mm, 1.70 μm) or on a Acquity HSS-T3 column (2.10×100 mm, 1.80 μm) using the Electrospray Ionisation (ESI) technique.

The following solvents, reagents or scientific terminology may be referred to by their abbreviations:

### TLC Thin Layer Chromatography

### DCM Dichloromethane

### THF Tetrahydrofuran

### MeOH Methanol

### EtOH Ethanol

IPA Isopropyl alcohol
EtOAc Ethyl acetate
Et₂O Diethyl ether

### DMA N,N-Dimethylacetamide

### DMF N,N-Dimethylformamide

### TEA/Et₃N Triethylamine

### DMSO Dimethylsulfoxide

DIPEA Diisopropylethylamine (Hunig's base)

### Mel Methyliodide

### NBS N-Bromosuccinimide

TBAB Tetrabutylammonium bromide
TBAI Tetrabutylammonium iodide
DIBAL-H Diisobutylaluminum hydride
TFA Trifluoroacetic acid
AcOH Acetic acid
Boc tert-butoxycarbonyl

### Cat Catalytic

mL milliliters
mmol millimoles
h hour or hours
min minute or minutes
g grams
mg milligrams

### μl Microlitres

### eq Equivalents

rt or RT Room temperature, ambient, about 27° C.
MS Mass spectrometry
Boc tert-Butyloxycarbonyl
m-CPBA meta-Chloroperbenzoic acid
T3P Propane phosphonic acid anhydride
BH₃-DMS Borane dimethylsulfide complex
LiBH₄ Lithium aluminum hydride
NaBH₄ Sodium borohydride

### H₂ Hydrogen

Pd/C Palladium on charcoal

### 1,2-DCE 1,2-Dichloroethane

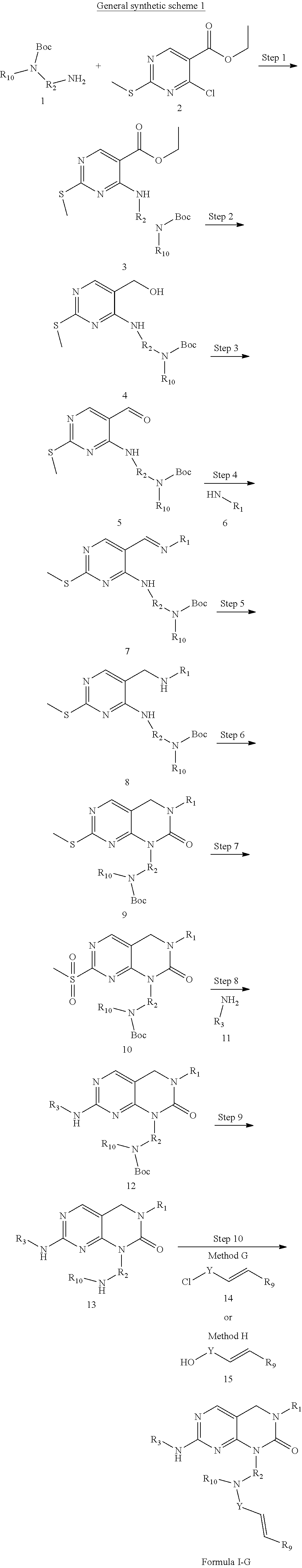

The chloropyrimidine derivative 2 is reacted with primary or secondary amine 1 in presence of base and solvent as DMF, DMA, IPA, MeOH, EtOH, preferably, DMF at −20° C. to 150° C., in order to produce compound 3. The ester group of compound 3 can be reduced using variety of reducing agents including DIBAL-H, BH₃-DMS, NaBH₄, LiALH₄, preferably, LiAlH₄ in presence of organic solvent such as THE to obtain compound 4. The alcohol group of compound 4 can be oxidized to aldehyde using activated manganese dioxide in presence of organic solvent such as dichloromethane, or ethylene dichloride to obtain aldehyde 5. The aldehyde compound 5 is reacted with variety of primary amines 6 in presence of base such as sodium acetate in presence of organic solvent, preferably methanol or ethanol to obtain imine derivative 7. Compound 7 can be reduced using variety of reducing agents including DIBAL-H, BH₃-DMS, NaBH₄, LiALH₄, H₂/Pd/C, preferably, NaBH₄ in presence of organic solvent, preferably, MeOH or EtOH or 1,2-dichloroethane in presence of acetic acid to yield compound 8. The diamine compound 8 cyclized using triphosgene or carbonyldiimidazole (CDI) in presence of base preferably diisopropyl ethylamine and in presence of organic solvent such as THE to afford 3,4-dihydropyrimido[4,5-d]pyrimidin-2(1H)-one derivative 9. The thiomethyl group in compound 9 can be oxidized using m-CPBA in presence of organic solvent such as DCM to obtain sulfone compound 10. The sulfone 10 reacted with different primary amines 11 and trifluoroacetic acid and in presence of solvent such as 2-BuOH, IPA, EtOH, MeOH, t-BuOH, preferably 2-BuOH at elevated temperature to obtain compound 12. In some cases of the invention, step 8 and 9 can happen as concerted reaction to yield compound 13 and mixture of compounds 12 and 13. The Boc compound 12 can be deprotected using different acids such as HCl or TFA in presence of organic solvent such as DCM, THF, or dioxane or mixture of solvents to obtain amine compound 13. Conversion of 13 to compound of Formula I-G was achieved either using compound 14, wherein different acid chlorides, preferably substituted acryloyl chloride in presence of base such as DIPEA, TEA, DMAP and using solvents such as DCM, THF, DMF, preferably, TEA and THE or DCM. In another method of obtaining compound of Formula I-G, compound 13 was treated with different acids 15 such as trans-N,N-dimethylaminocrotonic acid in presence of different coupling reagents such as T₃P, and base such as TEA and solvent such as DCM, THE to obtain compound of Formula I-G. In few examples of the invention the final compounds were isolated as salts of formic acid or TFA through prep HPLC purification and in few examples the racemic compounds were separated by chiral HPLC chromatography separation to get pure enantiomers.

When R₂ and R₁₀ forms a cyclic structure, similar procedure as described above was used for the synthesis of Formula I-G.

In another aspect of the invention, general procedures are provided, for the synthesis of 7, 8, 9, 10, 12, 13 compounds for use in the synthesis of a compound of Formula I-G. Further, general procedure for the synthesis of compound of Formula I-G is also provided.

A resulting compound of the disclosure is converted into any other compound of the disclosure by methods analogous to known methods. For example: a resulting compound of Formula I-G is converted into a salt or solvate thereof; the oxidation state of an atom in a heterocyclic ring is increased or decreased by oxidation or reduction using known methods.

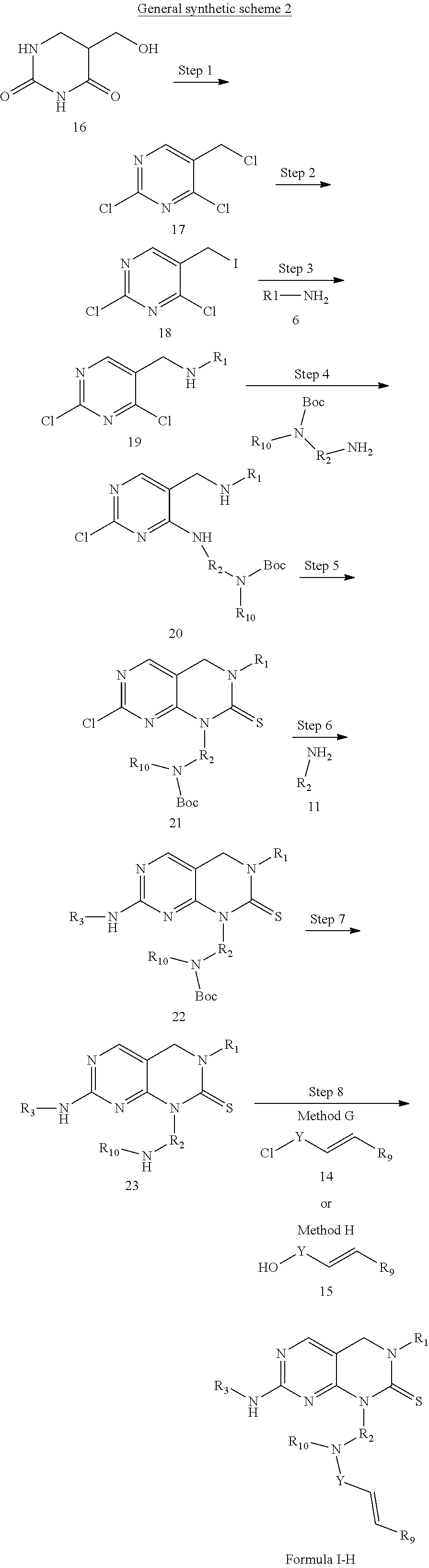

The hydroxyl pyrimidone derivative 16 is treated with POCl₃ in presence of base and solvent preferably toluene at 0° C. to 120° C. to obtain compound 17. The benzylic chloro compound 17 can be converted to corresponding iodo using sodium iodide in presence of organic solvent such as acetone to obtain compound 18. The iodo group of compound 18 can be reacted with the corresponding amines in presence of base such as NaOH and organic solvent preferably toluene to obtain compound 19. The compound 19 is reacted with variety of primary amines in presence of base such as diisopropylethyl amine and organic solvent preferably isopropyl alcohol to obtain diamine compound 20. The diamine compound 20 can be cyclized using thiophosgene in presence of base preferably diisopropyl ethylamine and in presence of organic solvent such as tetrahydrofuran to afford 3,4-dihydropyrimido[4,5-d]pyrimidin-2(1H)-thione derivative 21. The compound 21 can be treated with different primary amines in presence of organic or inorganic bases or acids such as hydrochloric acid, trifluoroacetic acid preferably trifluoroacetic acid in presence of organic solvent such as 2-butanol, isopropyl alcohol, ethanol, methanol, t-butanol, preferably 2-butanol at elevated temperature to obtain compound 22. In some cases of the invention, step 6 and 7 can happen as concerted reaction to yield compound 23 and mixture of compounds 22 and 23. The Boc compound 22 can be deprotected using different acids such as hydrochloric acid or trifluoroacetic acid in presence of organic solvent such as dichloromethane, tetrahydrofuran or dioxane or mixture of solvents to obtain amine compound 23. Conversion of 23 to compound of Formula I-G was achieved either using compound 14, wherein different acid chlorides, preferably, substituted acryloyl chloride in presence of base such as DIPEA, TEA, DMAP and using solvents such as DCM, THF, DMF, preferably TEA and THF or DCM. In another method of obtaining compound of Formula I-G, compound 23 was treated with different acids 15 such as trans-N,N-dimethylaminocrotonic acid in presence of different coupling reagents such as T₃P, and base such as TEA and solvent such as DCM, THF to obtain compound of Formula I-G and in few examples the racemic compounds were separated by chiral HPLC chromatography separation to get pure enantiomers.

When R₂ and R₁₀ forms a cyclic structure, similar procedure as described above was used for the synthesis of Formula I-G.

In another aspect of the invention, general procedures are provided, for the synthesis of compounds 17, 18, 19, 20, 21, 22 and 23 for use in the synthesis of a compound of Formula I-G. Further, general procedure for the synthesis of compound of Formula I-G is also provided.

A resulting compound of the disclosure is converted into any other compound of the disclosure by methods analogous to known methods. For example: a resulting compound of Formula I-G is converted into a salt or solvate thereof; the oxidation state of an atom in a heterocyclic ring is increased or decreased by oxidation or reduction using known methods.

### General Procedure A:

To a solution of aldehyde (5, 7.76 mmol) in MeOH or EtOH (30 mL) was added corresponding amine (6, 38.84 mmol) and sodium acetate (3.884 mmol) at RT and the reaction mixture was stirred for 4-20 h. The reaction was monitored by TLC. After the completion of reaction, the organic solvent was concentrated. The crude product was partitioned between DCM (3×200 mL) and water (50 mL). The organic layer was washed with brine (100 mL) and dried over anhydrous sodium sulfate and evaporated under reduced pressure to obtain crude product. The crude product was purified by silica gel chromatography to afford desired product 7.

### General Procedure B:

A solution of imine compound (7, 6.88 mmol) in MeOH or 1,2-dichloroethane (50 mL) was cooled to 0° C., then acetic acid (20 mL) was added and stirred for 10 min. Sodium borohydride (˜20.6 mmol) was added portion wise until starting material was consumed. The reaction was monitored by TLC. After the completion of reaction, the reaction mixture was slowly quenched with sodium bicarbonate solution. The crude reaction mixture was partitioned between DCM (2×200 mL) and water (50 mL). The organic layer was washed with brine (20 mL), dried over anhydrous sodium sulfate and evaporated under reduced pressure to obtain crude product. The crude product was purified by silica gel chromatography to provide pure product 8.

### General Procedure C:

To a solution of diamine compound (8, 5.98 mmol) in THE (35 mL) were added DIPEA (23.9 mmol) and triphosgene (2.15 mmol) at 0° C. The reaction mixture was stirred at RT for 2-12 h. The reaction was monitored by TLC. After completion of the reaction, sodium bicarbonate solution was added. The crude mixture was partitioned between DCM (2×100 mL) and water (50 mL). The organic layer was washed with brine (10 mL), dried over anhydrous sodium sulfate and evaporated under reduced pressure to obtain crude product. The crude product was purified by silica gel chromatography to get pure product 9.

### General Procedure D:

To a solution of thiomethyl compound (9, 4.18 mmol) in DCM (30 mL) was added m-CPBA (12.56 mmol) at RT. The reaction mixture was stirred at RT for 1-3 h. The reaction was monitored by TLC. After completion of the reaction, aqueous sodium bicarbonate solution was added. The crude mixture was partitioned between DCM (2×100 mL) and water (50 mL). The organic layer was washed with brine (10 mL) and dried over anhydrous sodium sulfate and evaporated under reduced pressure to obtain the crude product. The crude product was purified by recrystallization or by silica gel chromatography to afford desired sulfone derivative 10.

### General Procedure E:

To a solution of sulfone (10, 0.982 mmol) in 2-butanol (10 mL) was added corresponding amine (R2-amine) (11, 0.982 mmol) and TFA (1.17 mmol). The reaction mixture was heated for 3-16 h at 100-110° C. Reaction was monitored by TLC. After completion of the reaction, the reaction mixture was concentrated. Water (10 mL) and saturated sodium bicarbonate (20 mL) solution were added to the residue and extracted with DCM (3×200 mL). The combined organic layer was washed with brine solution (10 mL), dried over anhydrous sodium sulfate and evaporated under reduced pressure to obtain crude product. The crude product was purified by silica gel column chromatography to get pure product 12. In some examples step 8 and 9 was concerted to give a mixture of compounds 12 and 13 and in some cases isolated compound 13. In some examples, Chloro derivative (21) in place of sulfone derivative (10) was used to couple with R2-amine.

### General Procedure F:

To a solution of Boc compound (12, 0.507 mmol) in DCM (10 mL) and MeOH (3 mL) (some cases dioxane was used) cooled to 0° C., 4N HCl in dioxane (5.48 mmol) (some cases TFA was used) was added. The reaction mixture was stirred for 2-6 h at RT. The reaction was monitored by TLC. After completion of the reaction, water (10 mL) and saturated sodium bicarbonate (20 mL) solution were added and extracted with DCM (3×200 mL). The combined organic layer was washed with brine solution (10 mL), dried over anhydrous sodium sulfate and evaporated under reduced pressure to obtain crude product. The crude product was purified by silica gel column chromatography to get pure product 13.

### General Procedure G:

A solution of amine (13 or 23, 0.175 mmol) in DCM (10 mL) (some cases THE or mixture of DCM and THE was used) was cooled to 0° C. to −60° C., triethylamine (0.527 mmol) was added and stirred for 10 min. Then acryloyl chloride (0.527 mmol) was added and the reaction mixture was stirred for 0.5-6 h at 0° C. to −60° C. The reaction was monitored by TLC. After completion of the reaction, the reaction mixture was quenched with ice water at ˜−50° C. followed by sodium bicarbonate solution and extracted with DCM (100 mL). The organic layer was washed with brine solution (10 mL), dried over anhydrous sodium sulfate and evaporated under reduced pressure to obtain the crude product. The crude product was purified to get pure product of Formula I-G or I-H and in few examples the racemic compounds were separated by chiral preparative HPLC using Chiral Cel-OJH (20×250) mm, 5μ or Chiralpak IC (20×250) mm, 5μ column and n-hexane:EtOH as a mobile phase to get pure enantiomers.

### General Procedure H:

Amine (13 or 23, 0.34 mmol) and trans-N,N-dimethylaminocrotonic acid hydrochloride (0.40 mmol) were suspended in dichloromethane (10 mL) (some cases mixture of DCM and THE or THE was used), triethyl amine (0.1 mL) was added and cooled to 0° C. T₃P (0.68 mmol) was added drop-wise at 0° C. and the mixture was stirred at RT for 3-16 h. Completion of the reaction was monitored by TLC. The reaction mixture was partitioned between 5% methanol in dichloromethane and saturated bicarbonate solution. The organic layer was dried over anhydrous sodium sulfate, filtered and concentrated. The crude obtained was purified by silica gel chromatography to obtain pure product of Formula I-G or I-H and in few examples the racemic compounds were separated by chiral preparative HPLC to get pure enantiomers.

### General Procedure I:

A solution of hydroxy compound (16, 83.7 mmol) in POCl₃ (103 mL) was cooled to 0° C. and N,N-diisopropylethylamine (75 mL, 419.4 mmol) was added dropwise. The reaction mixture was heated for 14 h at 110° C. After the complete disappearance of starting material on TLC, the reaction mixture was quenched slowly with sodium bicarbonate solution. The resulting crude mixture was partitioned between ethyl acetate (200 mL) and water (50 mL). The organic layer was washed with brine (50 mL), dried over anhydrous sodium sulfate and evaporated under reduced pressure to obtain the crude product. The crude product was further purified by silica gel chromatography to provide pure product 17.

### General Procedure J:

Sodium iodide (14.06 mmol) was added to acetone (30 ml) and the mixture was stirred at RT until a clear solution was obtained. Then the chloro compound (17, 13.8 mmol) was added in single portion. The reaction mixture was stirred at RT for 30 min and heated to reflux for 20 min. The mixture was cooled to RT and the resulting white precipitate was filtered through sintered funnel and washed with acetone. The filtrate was concentrated at 30° C. The crude iodo compound 18 obtained was diluted with toluene and used directly for the next step.

### General Procedure K:

A solution of iodo compound (18, 0.35 mmol) in toluene (2 mL) was cooled to 0° C. and 4-fluoro aniline (0.35 mmol) was added. The reaction mixture was stirred for 30 min at 0° C. Then a solution of sodium hydroxide (0.45 mmol) in water (0.1 ml) was added and reaction mixture was stirred for 16 h at RT. The reaction was monitored by TLC. After completion of the reaction, water (3 mL) was added and extracted with ethyl acetate (2×10 mL). The organic layer was washed with brine solution (10 mL), dried over anhydrous sodium sulfate and evaporated under reduced pressure to obtain the crude residue. The crude compound was purified by silica gel column chromatography to get the pure product 19.

### General Procedure L:

To a stirred solution of dichloropyrimidine derivative (19, 0.07 mmol) in IPA (1 mL), corresponding amine (A-NH₂, 0.07 mmol) and DIPEA (0.3 mmol) were added. The reaction mixture was heated at 100° C. for 16 h in a sealed tube. Solvent was then evaporated and the crude thus obtained was purified by flash chromatography to obtain the desired product 20.

### General Procedure M:

To a solution of diamine compound (20, 5.98 mmol) in THE (35 mL) were added DIPEA (23.9 mmol) and thiophosgene (2.15 mmol) at 0° C. The reaction mixture was stirred at RT for 2-12 h with TLC monitoring. After completion of the reaction, sodium bicarbonate solution was added. The crude mixture was partitioned between DCM (2×100 mL) and water (50 mL). The organic layer was washed with brine (10 mL), dried over anhydrous sodium sulfate and evaporated under reduced pressure to obtain crude product. The crude product was purified by silica gel chromatography to get desired product 21.

### General Procedure N:

To a solution of compound of Formula I-G or I-H (0.087 mmol) in MeOH (15 ml) was added corresponding acid (0.087 mmol) at 10° C. Then the temperature raised to RT and stirred for 4 h. The organic solvent was removed under vacuum to get solid. The solid obtained was washed with n-pentane and dried over vacuum to get pure product of corresponding salt.

### General Procedure O:

To an ice-cold solution of ethyl 4-chloro-2-(methylsulfanyl)pyrimidine-5-carboxylate (1.0 eq) in dimethylformamide (10 volume) was added potassium carbonate (2.0 eq) and tert-butyl(3-aminophenyl)carbamate or analogues (1.2 eq) under nitrogen atmosphere. The resultant reaction mixture was heated at 80° C. for 16 hours. After completion of reaction (TLC monitoring), the reaction mixture was cooled to room temperature and poured into an ice-cold water. The resulting solid precipitate was filtered and dried under vacuum to get desired products.

### General Procedure P:

To a solution of products (1.0 eq) obtained from General procedure O in tetrahydrofuran (10 volume) was cooled to −40° C., followed by addition of lithium aluminum hydride (2.5 M in tetrahydrofuran, 3.0 eq) and the reaction mixture was stirred at room temperature for 16 hours. After completion of reaction (TLC monitoring), the reaction mixture was quenched with saturated ammonium chloride solution and stirred at room temperature for 30 minutes. The resulting reaction mixture was filtered and washed with dichloromethane. The filtrate was again washed with water and brine respectively. The organic layer was dried over anhydrous sodium sulfate, filtered and concentrated under reduced pressure afforded crude product. The crude product was triturated with dichloromethane afforded the desired products.

### General Procedure Q:

To a solution of products (1.0 eq) obtained from General procedure P in dichloromethane (10 volume) was added activated manganese dioxide (10.0 eq) at room temperature under nitrogen atmosphere. The resultant reaction mixture was stirred at same temperature for 16 hours. After completion of reaction (TLC monitoring), the reaction mixture was filtered through celite bed and washed with dichloromethane (3 times). The combined organic layer was dried over anhydrous sodium sulfate and concentrated under reduced pressure afforded the desired products.

### General Procedure R:

To a solution of products (1.0 eq) obtained from General procedure Q in methanol (10 volume) was added respective amines (3.0 eq) and sodium acetate (5.0 eq). The resultant reaction mixture was stirred at room temperature for 16 hours. After completion of reaction (monitored by TLC), the reaction mixture was poured in ice-cold water and resultant solid was filtered. The solid was dried under vacuum afforded the desired products.

### General Procedure S:

To a solution of products (1.0 eq) obtained from General procedure R in methanol (2.5 vol) was added acetic acid (1.0 vol) and sodium borohydride (1.0 eq). The resulting reaction mixture was stirred at room temperature for 16 hours. After completion of reaction (TLC monitoring), the reaction mixture was quenched with ice-cold water and resultant reaction mixture was filtered, washed with water. The solid was dried under vacuum afforded the desired products.

### General Procedure T:

To an ice-cold solution of products (1.0 eq) obtained from General procedure S in tetrahydrofuran (10 volume) was added N,N-diisopropylethylamine (4.0 eq) followed by the addition of triphosgene (0.4 eq) and the reaction mixture was stirred at room temperature for 16 hours. After completion of reaction (TLC monitoring) saturated sodium bicarbonate solution was added and extracted with dichloromethane (3 times). The organic layers were combined, washed with brine, dried over anhydrous sodium sulfate and evaporated under reduced pressure. The crude was triturated with diethyl ether afforded the desired products.

### General Procedure U:

To an ice-cold solution of products (1.0 eq) obtained from General procedure T in dichloromethane (10 volume) was added meta-chloroperoxybenzoic acid (2.0 eq) and the reaction mixture was stirred at room temperature for 4 hours. After completion of reaction (TLC monitoring), saturated solution of sodium bicarbonates was added to the reaction mixture and extracted with dichloromethane (3 times). The combined organic layers were washed with brine, dried over anhydrous sodium sulfate and evaporated under reduced pressure. The crude product was triturated with diethyl ether afforded the desired products.

### General Procedure V:

To an ice-cold solution of products (1.0 eq) obtained from General procedure U in isopropanol (10 volume) was added respective amines (1.2 eq) and trifluoroacetic acid (2.0 eq). The reaction mixture was heated at 110° C. for 16 hours. After completion of the reaction (TLC monitoring), the solvent was concentrated under reduced pressure, followed by saturated solution of sodium bicarbonate was added and extracted with dichloromethane (3 times). The combined organic layers were washed with brine solution, dried over anhydrous sodium sulfate and evaporated under reduced pressure afforded the crude product. The crude was triturated with diethyl ether afforded the desired products which was used directly for the next step without any further purification.

### General Procedure W:

An ice-cold solution of products (1.0 eq) obtained from General procedure V in 20% trifluoroacetic acid in dichloromethane (10 volume) was stirred at room temperature for 3-16 hours. After completion of the reaction (TLC monitoring), the solvent was evaporated to dryness. The reaction mixture diluted with saturated solution of sodium bicarbonate and extracted with 5% methanol in dichloromethane (3 times). The combined organic layers were washed with brine solution, dried over sodium sulfate and evaporated under reduced pressure. The crude was triturated with diethyl ether or purified over combiflash, eluted with 5-10% methanol in dichloromethane afforded the desired products.

### General Procedure X:

To an ice-cold solution of products (1.0 eq) obtained from General procedure W in dichloromethane (10 volume) was added triethylamine (5 eq), respective acids (1.1 eq), and propylphosphonic anhydride (T₃P, 50% in ethyl acetate, 2.5 eq). Then the reaction mixture was stirred at room temperature for 16 hours. After completion of reaction (TLC monitoring), reaction mixture was diluted with saturated solution of sodium bicarbonate and extracted with 5% methanol in dichloromethane. The combined organic layers were washed with brine, dried over anhydrous sodium sulfate, filtered and concentrated under reduced pressure afforded the crude product. The crude was purified over combiflash or Prep-TLC or Prep-HPLC afforded the final compounds.

### General Procedure Y:

A solution of products (1.0 eq) obtained from General procedure W in dichloromethane:tetrahydrofuran (1:1) (10 volume) was cooled to −70° C. followed by addition of triethylamine (5 eq) and acryloyl chloride (1.0 eq). The mixture was stirred at the same temperature for 2 hours. After completion of reaction (monitored by TLC), water was added and extracted with dichloromethane (3 times). The combined organic layers were washed with brine, dried over anhydrous sodium sulfate and concentrated under reduced pressure. The crudes were purified by Prep-HPLC afforded the final compounds.

### General Procedure Z:

To a solution of products (1.0 eq) obtained from General procedure W in tetrahydrofuran:water (3:1) (10 volume) at 0° C. was added triethylamine (3-5 eq) and acryloyl chloride (1.5 eq). The mixture was stirred at the same temperature for 2 hours. After completion of reaction (monitored by TLC), water was diluted with water and extracted with ethyl acetate (3 times). The combined organic layers were washed with brine, dried over anhydrous sodium sulfate and concentrated under reduced pressure. The crudes were purified by Prep-HPLC afforded the final compounds.

### General Procedure AA:

To an ice cold solution of nitro derivatives (1.0 eq) in methanol:tetrahydrofuran:water (2:2:1) (10 volume) was added zinc-dust or iron powder (5 eq) and ammonium chloride (5 eq). The resultant reaction mixture was stirred at room temperature for 2 hours. After completion of reaction (TLC monitoring), reaction mixture was passed through celite bed washed with 5% methanol in dichloromethane, filtrates were washed with water, brine, dried over anhydrous sodium sulfate and concentrated to dryness under reduced pressure afforded the amino derivatives.

### General Procedure BB (Suzuki Coupling):

To a solution of halo derivatives (1.0 eq) in acetonitrile (10 volume) was added respective boronate ester derivatives (1.0 eq), followed by a solution of potassium carbonate (2.0 eq) in water (3 volume) under argon purging. The resulting reaction mixture degassed for 15 minutes, followed by addition of [1,1′-bis(diphenylphosphino)ferrocene]dichloropalladium(II) dichloromethane complex (0.1 eq) and the reaction mixture was heated at 80° C. for 16 hours. After completion of reaction (TLC monitoring), diluted with ice water and extracted with ethyl acetate (3 times). The combined organic layers were washed with brine, dried over anhydrous sodium sulfate, filtered and evaporated to dryness under reduced pressure. The crude was purified over combiflash, elution with 40-60% ethyl acetate in hexane, desired fractions were concentrated under reduced pressure afforded the desired products.

### General Procedure CC:

To a solution of halo derivatives (1.0 eq) and respective boronic acids (1.1 eq) in toluene:ethanol (1:1) or dimethylformamide or dimethoxyethane (10 volume) was added a solution of potassium carbonate or sodium bicarbonate (2.0 eq) in water (3 volume) under argon degassing. The resulting reaction mixture was degassed for 15 minutes under argon atmosphere, followed by addition of [1,1′-bis(diphenylphosphino)ferrocene]dichloropalladium(II) (0.05 eq). The resulting reaction mixture was heated at 90° C. for 5-16 hours. After completion of reaction (TLC monitoring), the reaction mixture was cooled to room temperature, followed by water was added and extracted with ethyl acetate (3 times). The combined organic layers were washed with brine, dried over anhydrous sodium sulfate, filtered and concentrated under reduced pressure. The crude was purified over combiflash, eluted with 30-50% ethyl acetate in hexane, and desired fractions were concentrated under reduced pressure afforded the desired products.

### General Procedure DD:

To a solution of halo derivatives (1.0 eq) and respective boronate ester derivatives (1.1 eq) in acetonitrile:water (1:1) was added cesium carbonate (2.0 eq) under argon degassing. The resulting reaction mixture was degassed under argon atmosphere for 15 minutes, followed by the addition of tetrakis(triphenylphosphine)palladium(0) (0.1 eq). The resulting reaction mixture was heated at 90° C. for 16 hours. After completion of reaction (TLC monitoring), the reaction mixture was cooled to room temperature, treated with water, and extracted with ethyl acetate (3 times). The combined organic layers were washed with brine, dried over anhydrous sodium sulfate, filtered and concentrated under reduced pressure. The crude was purified using combiflash purifier, desired fractions were concentrated under reduced pressure afforded the desired products.

### General Procedure EE:

To an ice-cold solution of N-(3-(2-chloro-6-fluoroquinazolin-8-yl)phenyl)acrylamide (1.0 eq) in dimethylformamide was added sodium hydride (10.0 eq) portion-wise and stirred at the same temperature for 30 minutes, followed by addition of respective amines (1.2 eq). The resultant reaction mixture was stirred at room temperature for 16 hours. After completion of the reaction (as per TLC monitoring), reaction mixture was quenched with ice-cold water and extracted with 5% methanol/dichloromethane (3 times). The organic layers were combined and dried over anhydrous sulfate, filtered and concentrated under reduced pressure to obtain the crude product. The crude was purified over combiflash or Prep HPLC purification afforded the desired products.

### General Procedure FF:

To a solution of nitro derivatives (1.0 eq) in methanol (10 vol) was added 10% palladium on carbon (20% w/w). The reaction mixture was stirred under hydrogen atmosphere for 16 hours. After completion of reaction (TLC monitoring), reaction mixture was filtered through celite bed and washed with methanol. The filtrate was concentrated under reduced pressure afforded amino derivatives.

### Synthesis of Intermediate Amines [A-NH₂ (1), R1-NH₂ (6) and R2-NH₂ (11)]

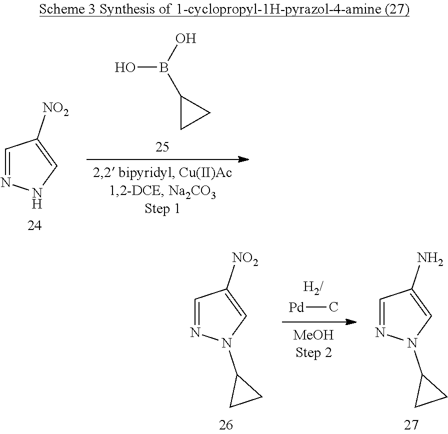

### Step 1: 1-cyclopropyl-4-nitro-1H-pyrazole (26)

4-nitro-1H-pyrrazole (24, 2.0 g, 18 mmol), cyclopropyl boronic acid (25, 3.0 g, 35 mmol), 2,2′-bipyridine (2.8 g, 18 mmol) and sodium carbonate (3.8 g, 35 mmol) were mixed in round bottom flask containing 1,2-dichloroethane (80 mL). The mixture was purged with oxygen gas for 10 min, copper (II) acetate (3.2 g, 18 mmol) was added, purged further for 15 min and heated at 80° C. for 6 h under oxygen atmosphere. The mixture was then passed through a celite pad, the filtrate was partitioned between dichloromethane and water. The organic layer was dried over anhydrous sodium sulfate, filtered and concentrated to obtain a crude mass. The crude mass was purified by flash column chromatography on silica gel (using 12 g SNAP column, and 8% ethyl acetate in hexane on a Biotage chromatography system) to give 1-cyclopropyl-4-nitro-1H-pyrazole (26, 41% yield) as a white solid. ¹H NMR (400 MHz, CDCl₃): δ 8.18 (s, 1H), 7.97 (s, 1H), 3.70-3.64 (m, 1H), 1.03-1.02 (m, 2H), 0.88-0.86 (m, 2H).

### Step 2: 1-cyclopropyl-1H-pyrazol-4-amine (27)

1-cyclopropyl-4-nitro-1H-pyrazole (26, 1.10 g, 7.2 mmol) was dissolved in methanol (20 mL) under nitrogen atmosphere and 10% Pd on C (50% moisture, 0.4 g) was added to it. The mixture was subjected to hydrogenation under balloon pressure for 6 h. The catalyst was removed by filtering through celite and the filtrate was concentrated to obtain 1-cyclopropyl-1H-pyrazol-4-amine (27, 97% yield) as a brown liquid. H NMR (400 MHz, DMSO-d₆): δ 7.00-6.99 (d, 1H), 6.78-6.77 (d, 1H), 3.82 (s, 2H), 3.51-3.49 (m, 1H), 0.99-0.92 (m, 2H), 0.86-0.80 (m, 2H).

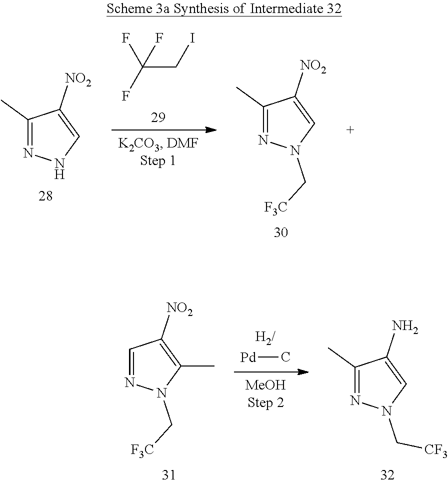

### Step 1: 3-methyl-4-nitro-1-(2,2,2-trifluoroethyl)-1H-pyrazole (30) and 5-methyl-4-nitro-1-(2,2,2-trifluoroethyl)-1H-pyrazole (31)

3-methyl-4-nitro-1H-pyrrazole (28, 1.0 g, 7.8 mmol), trifluoroethyl iodide (29, 3.3 g, 15.7 mmol) and potassium carbonate (1.64 g, 11.9 mmol) were mixed in a vial containing DMF (5 mL). The vial was closed and the mixture was heated at 60° C. for 3 days. Water was added and extracted with ethyl acetate. The organic extract was dried over anhydrous sodium sulfate, filtered and solvents evaporated to obtain a sticky mass, purification of which by flash column chromatography on silica gel (using 12 g SNAP column and 0-10% ethyl acetate in hexane as eluent on a Biotage chromatography system) yielded 3-methyl-4-nitro-1-(2,2,2-trifluoroethyl)-1H-pyrazole (30, 29% yield) as major product and 5-methyl-4-nitro-1-(2,2,2-trifluoroethyl)-1H-pyrazole (31) as minor product. ¹H NMR (400 MHz, CDCl₃): δ 8.25 (s, 1H), 4.69-4.63 (q, 2H), 2.56 (s, 3H).

### Step 2: 3-methyl-1-(2,2,2-trifluoroethyl)-1H-pyrazol-4-amine (32)

3-methyl-4-nitro-1-(2,2,2-trifluoroethyl)-1H-pyrazole (30, 0.460 g, 2.2 mmol) was dissolved in methanol (20 mL) under nitrogen atmosphere and 10% Pd on C (50% moisture, 0.25 g) was added. The mixture was hydrogenated under balloon pressure for 5 h. The catalyst was removed by filtering through celite and the filtrate was concentrated to obtain 3-methyl-1-(2,2,2-trifluoroethyl)-1H-pyrazol-4-amine (32, 91% yield) as a pink solid. ¹H NMR (400 MHz, CDCl₃): δ 7.03 (s, 1H), 4.53-4.47 (q, 2H), 2.81 (s, 2H), 2.18 (s, 3H).

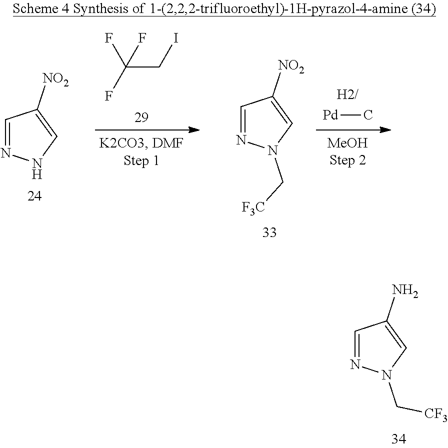

### Step 1: 4-nitro-1-(2,2,2-trifluoroethyl)-1H-pyrazole (33)

Title compound was prepared in a manner substantially similar to procedure mentioned in step 1 of Scheme 3a.

4-nitro-1H-pyrrazole (24) and trifluoroethyl iodide (29) gave 4-nitro-1-(2,2,2-trifluoroethyl)-1H-pyrazole (33, 42% yield) as an off white solid. ¹H NMR (400 MHz, CDCl₃): δ 8.28 (s, 1H), 8.15 (s, 1H), 4.79-4.73 (q, 2H).

### Step 2: 1-(2,2,2-Trifluoroethyl)-1H-pyrazol-4-amine (34)

Title compound was prepared in a manner substantially similar to procedure mentioned in step 2 of scheme 3a.

4-Nitro-1-(2,2,2-trifluoroethyl)-1H-pyrazole (33) gave 1-(2,2,2-trifluoroethyl)-1H-pyrazol-4-amine (34) as a light brown sticky mass. ¹H NMR (400 MHz, CDCl₃): δ 7.25 (s, 1H), 7.09 (s, 1H), 4.60-4.54 (q, 2H), 2.96 (bs, 2H).

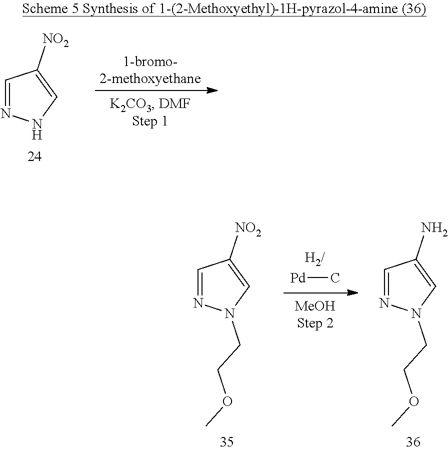

### Step 1: 1-(2-Methoxyethyl)-4-nitro-1H-pyrazole (35)

Title compound was prepared in a manner substantially similar to procedure mentioned in step 1 of Scheme 3a.

3-Methyl-4-nitro-1H-pyrrazole (24) and 1-bromo-2-methoxyethane gave 1-(2-methoxyethyl)-4-nitro-1H-pyrazole (35, 66% yield) as an off white solid. ¹H NMR (400 MHz, CDCl₃): δ 8.85 (s, 1H), 8.27 (s, 1H), 4.36-4.33 (t, 2H), 3.77-3.72 (t, 2H), 3.23 (s, 3H).

### Step 2: 1-(2-Methoxyethyl)-1H-pyrazol-4-amine (36)

Title compound was prepared in a manner substantially similar to procedure mentioned in step 2 of Scheme 3a. 1-(2-Methoxyethyl)-4-nitro-1H-pyrazole (35) gave 1-(2-Methoxyethyl)-1H-pyrazol-4-amine (36, 91% yield) as a pink solid. ¹H NMR (400 MHz, CDCl₃): δ 7.03 (s, 1H), 6.89 (s, 1H), 4.19-4.17 (t, 2H), 3.78 (bs, 2H), 3.67-3.64 (t, 2H), 3.21 (s, 3H).

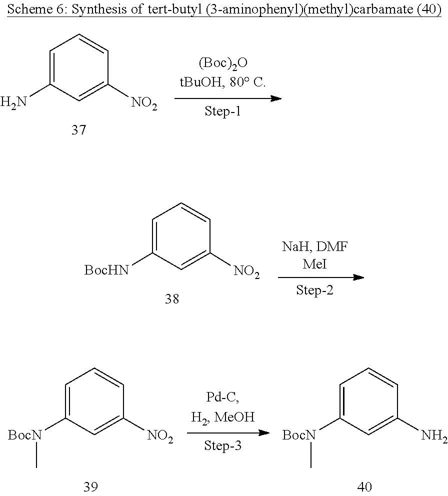 [SMILES: Nc1cccc([N+](=O)[O-])c1 ; CC(C)(C)OC(=O)Nc1cccc([N+](=O)[O-])c1 ; CN(C(=O)OC(C)(C)C)c1cccc([N+](=O)[O-])c1 ; CN(C(=O)OC(C)(C)C)c1cccc(N)c1]

### Step 1: tert-butyl (3-nitrophenyl)carbamate (38)

(Boc)₂O (95 mL, 434.4 mmol) was added to 3-nitroaniline (37, 50.0 g, 362.0 mmol) in ^{t}BuOH (30 mL) at 0° C. The reaction mixture was heated at 80° C. for 16 h and concentrated under reduced pressure. The crude was washed with pentane (5×30 mL) to afford the title compound as a light yellow solid (38, 85.0 g, 98% yield). ¹H NMR (400 MHz, CDCl₃): δ 8.30 (t, 1H), 7.88 (dd, 1H), 7.68 (d, 1H), 7.42-7.46 (m, 1H), 6.73 (bs, 1H), 1.53 (s, 9H).

### Step 2: tert-butyl methyl(3-nitrophenyl)carbamate (39)

Sodium hydride (3.5 g, 83.9 mmol) was taken in a 500 mL round bottom flask under nitrogen atmosphere, DMF was added and cooled to 0° C. To it was added tert-butyl (3-nitrophenyl)carbamate (38, 10.0 g, 42.0 mmol) and the reaction mixture was stirred for another 30 min at 0° C. Iodomethane (4.0 mL, 63.0 mmol) was added and the reaction mixture was stirred at room temperature for 12 h. The reaction mixture was quenched with ice-water (50 mL) and extracted with EtOAc (3×100 mL). The combined organic layer was dried over anhydrous Na₂SO₄ and concentrated under reduced pressure. The crude product was washed with Et₂O (3×50 mL) to afford the title compound as a light yellow solid (39, 8.0 g, 86% yield). ¹H NMR (400 MHz, CDCl₃): δ 8.15 (s, 1H), 8.01-7.98 (m, 1H), 7.62 (d, 1H), 7.48 (t, 1H), 3.32 (s, 3H), 1.48 (s, 9H).

### Step 3: tert-butyl (3-aminophenyl)(methyl)carbamate (40)

A par flask was charged with tert-butyl methyl(3-nitrophenyl)carbamate (39, 8.0 g, 1.9 mmol) and methanol (20 mL) followed by addition of Pd—C (10% w/w, 0.8 g). The flask was evacuated under vacuum and then purged with hydrogen. The reaction was stirred under hydrogen atmosphere (30 psi). The reaction was monitored by TLC. It was then filtered through sintered funnel with a pad of celite, washed with methanol and concentrated under reduced pressure to afford the title compound as a deep brown liquid (40, 0.35 g, 64% yield) that was used as such for the next step without any further purification. LCMS: 223.14 (M+H)⁺

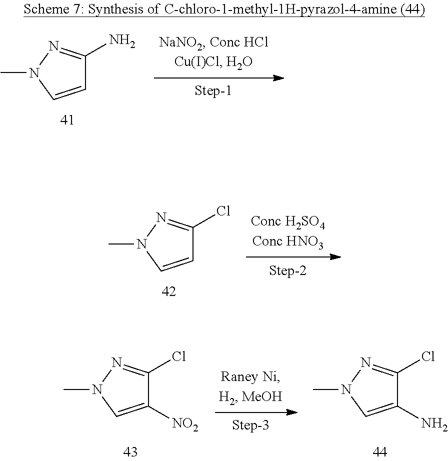

### Step 1: 3-chloro-1-methyl-1H-pyrazole (42)

1-methyl-1H-pyrazol-3-amine (41, 5.0 g, 51.5 mmol) was taken in Conc. HCl (50 mL) in a 250 mL round bottom flask and cooled to 0° C. A solution of sodium nitrite (5.3 g, 77.2 mmol) in H₂O (100 mL) was added slowly. The reaction mixture was stirred at room temperature for 30 min. Finally, Cu(I)Cl (10.2 g, 100.0 mmol) was added and the reaction mixture was heated at 60° C. for 1 h. The reaction was quenched with ice-cooled 50% sodium hydroxide solution (100 mL) and extracted with EtOAc (3×100 mL). The combined organic layer was dried over anhydrous Na₂SO₄, filtered and concentrated under reduced pressure to afford the title compound as a brown liquid (42, 6.2 g, 52% yield). ¹H NMR (400 MHz, CDCl₃): δ 7.28 (d, 1H), 6.15 (d, 1H), 3.85 (s, 3H).

### Step 2: 3-chloro-1-methyl-4-nitro-1H-pyrazole (43)

3-chloro-1-methyl-1H-pyrazole (42, 6.2 g, 53.4 mmol) was taken in Conc. H₂SO₄ (10 mL) in a 100 mL round bottom flask under nitrogen atmosphere and cooled to 0° C. Fuming HNO₃ (8.0 mL, 187.0 mmol) was added drop wise maintaining the internal bath temperature at 0° C. After the addition was complete, the reaction mixture was stirred at room temperature for 2 h. The reaction mixture was quenched with ice-water (40 g) and diluted with EtOAc (300 mL). The organic layer was washed with saturated NaHCO₃ solution (3×100 mL), dried over anhydrous Na₂SO₄ and concentrated under reduced pressure. The crude was washed pentane (3×20 mL) to afford the title compound as a white solid (43, 5.2 g, 60% yield) that was used as such for the next step without any further purification. ¹H NMR (400 MHz, DMSO-d₆): δ 8.97 (s, 1H), 3.88 (s, 3H).

### Step 3: 3-chloro-1-methyl-1H-pyrazol-4-amine (44)

A par flask was charged with 3-chloro-1-methyl-4-nitro-1H-pyrazole (43, 5.2 g, 32.2 mmol) and methanol (100 mL) followed by addition of Raney Ni (10% w/w, 0.5 g). The flask was evacuated under vacuum and then purged with hydrogen. The reaction was stirred under hydrogen atmosphere (30 psi) for 2 days. The reaction was monitored by TLC. It was then filtered through sintered funnel with a pad of celite and washed with methanol and concentrated under reduced pressure to obtain the title compound as a brown colored liquid (44, 4.0 g, 94% yield). ¹H NMR (400 MHz, DMSO-d₆): δ 7.09 (s, 1H), 3.88 (bs, 2H), 3.64 (s, 3H).

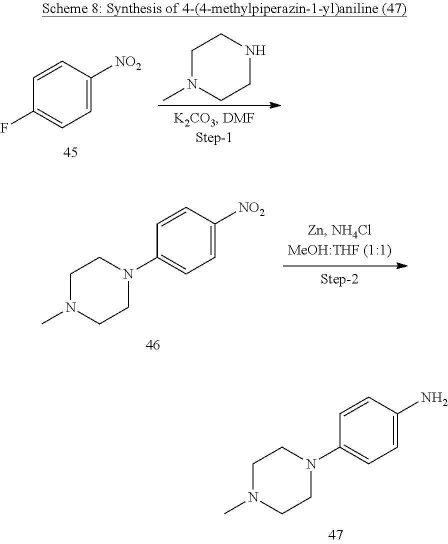

### Step 1: 1-methyl-4-(4-nitrophenyl)piperazine (46)

1-fluoro-4-nitrobenzene (45, 25.0 g, 177.1 mmol) was taken in DMF (100 mL) in a 250 mL round bottom flask under nitrogen atmosphere and added 1-methylpiperazine (16.8 g, 168.0 mmol) and K₂CO₃ (36.7 g, 265.0 mmol). The reaction mixture was heated at 80° C. for 16 h. The reaction mixture was then poured onto ice water and extracted with EtOAc (3×100 mL). The combined organic layer was dried over anhydrous Na₂SO₄ and concentrated under reduced pressure. The crude was purified by column chromatography on silica gel (100-200 mesh) using 50% EtOAc in hexane as eluent to afford the title compound as a yellow solid (46, 35.0 g, 89% yield). ¹H NMR (400 MHz, DMSO-d₆): δ 8.04 (d, 2H), 7.02 (d, 2H), 3.45-3.42 (m, 4H), 2.43-2.40 (m, 4H), 2.21 (s, 3H).

### Step 2: 4-(4-methylpiperazin-1-yl)aniline (47)

1-methyl-4-(4-nitrophenyl)piperazine (46, 5.1 g, 22.0 mmol) was taken in a 500 mL round bottom flask in a mixture of solvent MeOH and THF (100 mL, 1:1). NH₄Cl (12.0 g, 220.0 mmol) and Zn dust (14.7 g, 220.0 mmol) were added and the reaction mixture was stirred at room temperature for 2 h. It was then filtered through sintered funnel with a pad of celite, washed with MeOH (50 mL) and concentrated under reduced pressure. The crude was further washed with n-pentane to afford 47 as a gummy residue (4.0 g, 93% yield). ¹H NMR (400 MHz, DMSO-d₆): δ 7.34 (bs, 2H), 6.75-6.73 (m, 2H), 6.56-6.54 (m, 2H), 3.20 (m, 8H), 2.73 (s, 3H).

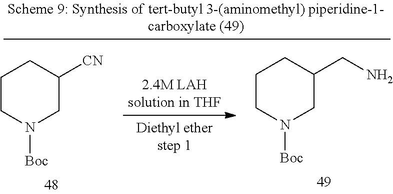 [SMILES: CC(C)(C)OC(=O)N1CCCC(C#N)C1 ; CC(C)(C)OC(=O)N1CCCC(CN)C1]

### Step 1: tert-butyl 3-(aminomethyl) piperidine-1-carboxylate (49, Intermediate-6)

tert-butyl 3-cyanopiperidine-1-carboxylate (48, 5.0 g, 23.1 mmol) was taken in diethyl ether (50 mL) in a 250 mL round bottom flask under nitrogen atmosphere. The reaction mixture was cooled to −78° C. and 2.4M LAH solution in THE (19.8 mL, 47.0 mmol) was added drop wise. After the addition was complete, the reaction mixture was then stirred at room temperature for 2 h. The reaction was monitored by TLC. After the completion of reaction, the reaction mixture was quenched with 10% NaOH solution at 0° C. drop wise and organic layer was separated. The solvent was concentrated under reduced pressure to afford the title compound as a brown liquid (49, 4.6 g, 92% yield). LCMS Calcd for [M+H]⁺ 215.3 found 215.12

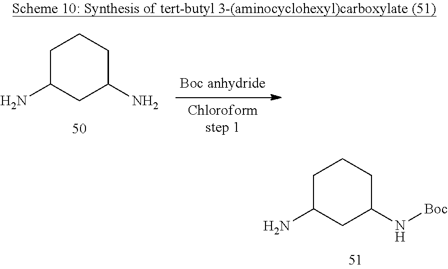 [SMILES: NC1CCCC(N)C1 ; CC(C)(C)OC(=O)NC1CCCC(N)C1]

### Step 1: tert-butyl (3-aminocyclohexyl)carbamate (51)

Cyclohexane-1,3-diamine (51, 4.0 g, 35.1 mmol) was taken in chloroform (160 mL) in a 250 mL round bottom flask under nitrogen atmosphere. The reaction mixture was cooled to −5° C. and a solution of Boc anhydride (3.3 mL, 14.0 mmol) in chloroform (60 mL) was added drop wise. After the addition was complete, the reaction mixture was then stirred at 0° C. for 6 h. The reaction was monitored by TLC (Ninhydrin active). The reaction mixture was then poured onto ice water and extracted with DCM (2×50 mL). The combined organic layer was dried over anhydrous Na₂SO₄ and concentrated under reduced pressure to afford the title compound as a white sticky liquid (51, 4.5 g, 53% yield). Mass Calcd for [M+H]⁺ 215.3 direct mass found 215.12

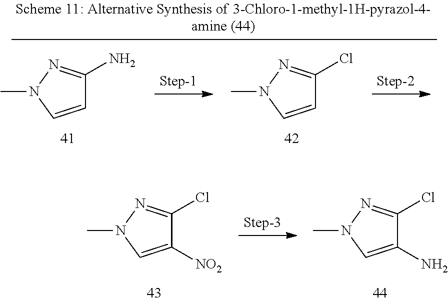

### Step 1: Preparation of 3-Chloro-1-methyl-1H-pyrazole (42)

To an ice cold solution of 1-methyl-1H-pyrazol-3-amine (41) (50 g, 0.52 mol) in acetonitrile (400 mL) was added copper(I) chloride (154 g, 1.56 mol). The resulting mixture was stirred at room temperature for 30 minutes, followed by tert-butyl nitrite (268 g, 2.60 mol) was added and stirred at 60° C. for 30 minutes. After completion of the reaction (TLC monitoring), the reaction mixture was poured into water and extracted with ethyl acetate (3×300 mL). The combined organic layers were washed with brine and dried over anhydrous sodium sulfate and concentrated under reduced pressure afforded desired product (42) (32.0 g; Yield: 53%). ¹H-NMR (400 MHz, CDCl₃): δ 7.27 (d, J=2.4 Hz, 1H), 6.15 (d, J=2.4 Hz, 1H), 3.85 (s, 3H).

### Step 2: Preparation of 3-Chloro-1-methyl-4-nitro-1H-pyrazole (43)

To an ice-cold solution of 3-chloro-1-methyl-1H-pyrazole (42) (30 g, 0.26 mol) in concentrated sulfuric acid (50 mL) was slowly added fuming nitric acid (40 mL, 0.91 mol) drop wise. The resulting mixture was stirred at room temperature for 6 hours. After completion of reaction (TLC monitoring), the reaction mixture was poured into ice-cold water, the resulting solid was filtered and washed with pentane afforded the desired product (43) as yellow solid (30 g; Yield: 73%). ¹H-NMR (400 MHz, CDCl₃): δ 8.16 (s, 1H), 3.94 (s, 3H).

### Step 3: Preparation of 3-Chloro-1-methyl-1H-pyrazol-4-amine (44)

To a solution of 3-chloro-1-methyl-4-nitro-1H-pyrazole (43) (30 g, 0.186 mol) in methanol (300 mL) was added Raney nickel (10% w/w, 3 g). The reaction was stirred under hydrogen atmosphere for 16 hours. The reaction was monitored by TLC (after completion), the reaction mixture was filtered through celite bed and washed with methanol. The filtrate was concentrated under reduced pressure afforded 44 as viscous liquid (14.0 g; Yield: 57%). H-NMR (400 MHz, DMSO-d₆): δ 7.09 (s, 1H), 3.88 (s, 2H), 3.64 (s, 3H).

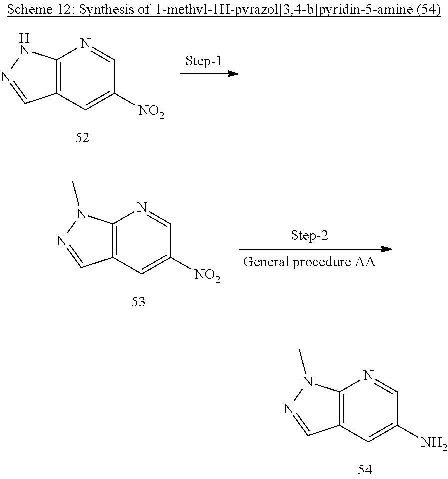

### Step 1: Preparation of 1-Methyl-5-nitro-1H-pyrazolo[3,4-b]pyridine (53)

To an ice-cold solution of 5-nitro-1H-pyrazolo[3,4-b]pyridine (52) (3.0 g, 18.3 mmol) in dimethylformamide (50 mL) was added sodium hydride (60% dispersion in mineral oil, 0.88 g, 21.9 mmol) in portion wise. The resulting mixture was stirred at same temperature for 15 minutes, followed by the addition of methyl iodide (1.25 mL, 20.1 mmol) and stirred at 0° C. for 2 hours. After completion of reaction (TLC monitoring), the reaction mixture was poured into ice-cold water and extracted with ethyl acetate (3×100 mL). The combined organic layers were dried over anhydrous sodium sulfate and concentrated under reduced pressure afforded the crude product. The crude was purified by column chromatography on silica gel (100-200 mesh) eluted with 90% ethyl acetate-hexane as an eluent afforded 53 as white solid (2.0 g; Yield: 61%). LCMS: [M+H]⁺ 179.18; 99.58%

### Step 2: Preparation of 1-Methyl-1H-pyrazolo[3,4-b]pyridin-5-amine (54)

Title compound was prepared in a manner substantially similar to procedure mentioned in General procedure AA. The crude was washed with n-pentane to afford (54) as light brown solid (1.0 g; Yield: 60%). ¹H-NMR (400 MHz, DMSO-d₆): δ 8.08 (d, J=4.2 Hz, 1H), 7.79 (s, 1H), 7.17 (d, J=4.2 Hz, 1H), 5.09 (s, 2H), 3.95 (s, 3H). LCMS: [M+H]⁺ 149.15; 93.75%.

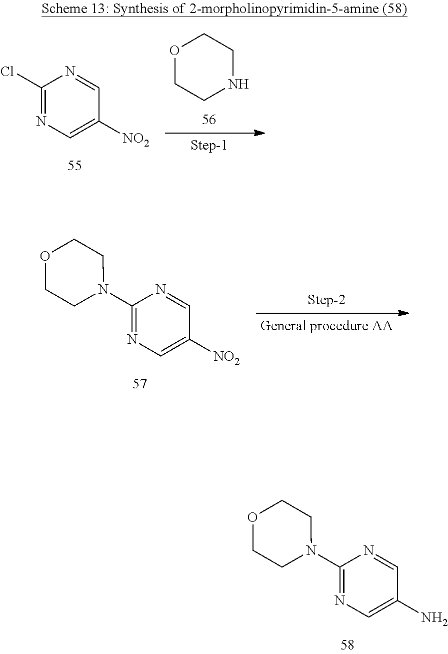 [SMILES: O=[N+]([O-])c1cnc(Cl)nc1 ; C1COCCN1 ; O=[N+]([O-])c1cnc(N2CCOCC2)nc1 ; Nc1cnc(N2CCOCC2)nc1]

### Step 1: Preparation of 4-(5-nitropyrimidin-2-yl)morpholine (57)

To an ice-cold solution of morpholine (56) (1.64 g, 18.8 mmol) in tetrahydrofuran (75 mL) was added triethylamine (5.24 mL, 37.6 mmol). The resulting mixture was stirred at same temperature for 15 minutes, followed by 2-chloro-5-nitropyrimidine (55) (3.0 g, 18.8 mmol) was added and stirred at room temperature for 12 hours. After completion of reaction (TLC monitoring), the reaction mixture was poured into water and extracted with ethyl acetate (3×100 mL). The combined organic layers were dried over anhydrous sodium sulfate and concentrated under reduced pressure afforded 57 as yellow solid (3.4 g; Yield: 86%). ¹H-NMR (400 MHz, DMSO-d₆): δ 9.12 (s, 2H), 3.92-3.89 (m, 4H), 3.70 (m, 4H). LCMS: [M+H]⁺ 211.21; 99.47%.

### Step 2: Preparation of 2-morpholinopyrimidin-5-amine (58)

Title compound was prepared as pale yellow solid (2.0 g; Yield: 65%) in a manner substantially similar to procedure mentioned in General procedure AA. ¹H-NMR (400 MHz, DMSO-d₆): δ 7.90 (s, 2H), 4.64 (s, 2H), 3.64-3.62 (m, 4H), 3.45 (m, 4H). LCMS: [M+H]⁺ 181.04, 96.0%

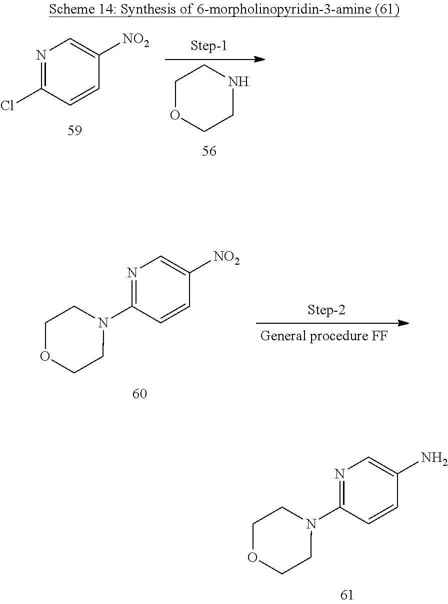 [SMILES: O=[N+]([O-])c1ccc(Cl)nc1 ; C1COCCN1 ; O=[N+]([O-])c1ccc(N2CCOCC2)nc1 ; Nc1ccc(N2CCOCC2)nc1]

### Step 1: Preparation of 4-(5-nitropyridin-2-yl)morpholine (60)

To an ice-cold solution of morpholine (56) (5.5 g, 63.1 mmol) in dichloromethane (50 mL) was added triethylamine (8.5 mL, 63.1 mmol). The resulting mixture was stirred at room temperature for 15 minutes, followed by addition of 2-chloro-5-nitropyridine (59) (10 g, 63.1 mmol) and stirred at room temperature for 3 hours. After completion of reaction (TLC monitoring), the reaction mixture was poured into water and extracted with dichloromethane (3×100 mL). The combined organic layers were dried over anhydrous sodium sulfate and concentrated under reduced pressure afforded 60 as yellow solid (6.0 g, Yield: 45%). ¹H-NMR (400 MHz, DMSO-d₆): δ 8.97 (d, J=8.0 Hz, 1H), 8.25 (d, J=5.0 Hz, 1H), 6.95 (d, J=5.0 Hz, 1H), 3.82-3.70 (m, 8H). LCMS: [M+H]⁺ 210.14; 99.78%.

### Step 2: Preparation of 6-morpholinopyridin-3-amine (61)

Title compound was prepared as a brown solid (2.7 g: Yield: 62%) in a manner substantially similar to procedure mentioned in General procedure FF. ¹H-NMR (400 MHz, CDCl₃): δ 7.82 (m, 1H), 7.05-7.02 (d, J=5.02 Hz, 1H), 6.60 (d, J=5.12 Hz 1H), 3.86-3.84 (m, 4H), 3.35 (m, 4H). LCMS: [M+H]⁺ 180.12; 99.39%.

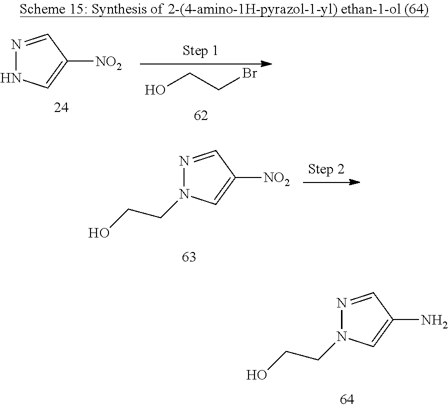

### Step 1: Preparation of 2-(4-nitro-1H-pyrazol-1-yl) ethan-1-ol (63)

To an ice-cold solution of 4-nitro-1H-pyrazole (24) (10 g, 88.4 mmol) in dimethylformamide (130 mL) was added cesium carbonate (43.5 g, 133 mmol) and 2-bromoethan-1-ol (62) (12.2 g, 97.3 mmol) and the reaction mixture was heated at 100° C. for 8 hours. After completion of reaction (TLC monitoring), the reaction mixture was cooled to room temperature, poured into water and extracted with ethyl acetate (2×200 mL). The combined organic layers were washed with water, dried over anhydrous sodium sulfate and concentrated under reduced pressure afforded 63 as a brown solid (6.0 g; Yield: 41%). ¹H-NMR (400 MHz, DMSO-d₆): δ 8.81 (s, 1H), 8.26 (s, 1H), 4.99 (m, 1H), 4.22 (m, 2H), 3.76 (m, 2H).

### Step 2: Preparation of 2-(4-amino-1H-pyrazol-1-yl) ethan-1-ol (64)

To a solution of 2-(4-nitro-1H-pyrazol-1-yl) ethan-1-ol (63) (6.0 g, 38.2 mmol) in ethanol (50 mL) was added 20% palladium hydroxide (10% w/w, 0.6 g) at room temperature and the reaction mixture was stirred under hydrogen atmosphere for 14 hours. After completion of reaction (TLC monitoring), the reaction mixture was filtered through celite and washed with methanol (100 mL), the filtrate was concentrated under reduced pressure to afford (64) as a brown solid (4.0 g; Yield: 81%). MS: [M+H]⁺ 128.07.

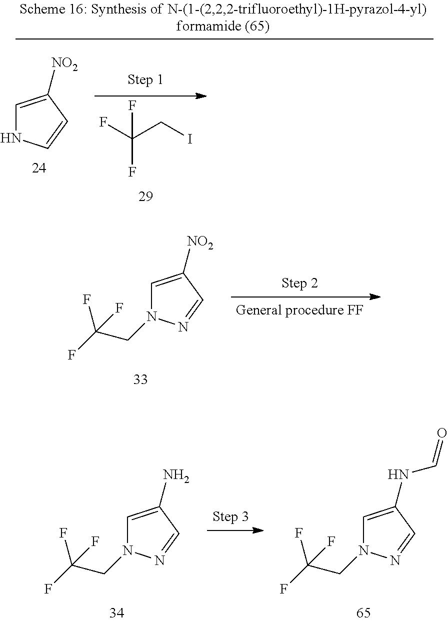

### Step 1: Preparation of 4-nitro-1-(2,2,2-trifluoroethyl)-1H-pyrazole (33)

To a solution of 4-nitro-1H-pyrazole (24) (5.0 g, 44.21 mmol) in dimethylformamide (25 mL) was added cesium carbonate (17.3 g, 53.05 mmol) and 1,1,1-trifluoro-2-iodoethane (29) (14 g, 66.31 mmol). The resulting mixture was heated at 50° C. for 16 hours. After completion of reaction (TLC monitoring), the reaction mixture was cooled to room temperature, poured into water and extracted with ethyl acetate (3×100 mL). The combined organic layers were washed with brine, dried over anhydrous sodium sulfate and concentrated under reduced pressure. The crude was purified over column chromatography over silica gel (100-200 mesh) using 10% ethyl acetate-hexane as eluent afforded 33 as an off white solid (2 g; Yield: 23%). ¹H-NMR (400 MHz, CDCl₃): δ 8.28 (s, 1H), 8.15 (s, 1H), 4.79 (m, 2H).

### Step 2: Preparation of 1-(2,2,2-trifluoroethyl)-1H-pyrazol-4-amine (34)

Title compound was prepared in a manner substantially similar to the procedure mentioned in General procedure FF, afforded 34 as a light brown sticky mass (1.5 g: Yield: 89%). ¹H-NMR (400 MHz, CDCl₃): δ 7.25 (s, 1H), 7.05 (s, 1H), 4.60 (m, 2H), 2.96 (bs, 2H).

### Step 3: Preparation of N-(1-(2,2,2-trifluoroethyl)-1H-pyrazol-4-yl) formamide (65)

A mixture of acetic anhydride (2.3 mL, 3.88 mmol) and formic acid (1.0 mL, 8.69 mmol) were heated at 70° C. for 1 hour to prepare the formylating mixture. This reaction mixture was then gradually cooled to 0° C. and 1-(2,2,2-trifluoroethyl)-1H-pyrazol-4-amine (34) (400 mg, 2.42 mmol) in tetrahydrofuran (10 mL) was slowly added. The resulting mixture was stirred at room temperature for 2 hours. After completion of reaction (TLC monitoring), the reaction mixture was poured into water and extracted with ethyl acetate (3×50 mL). The combined organic layers were washed with brine, dried over anhydrous sodium sulfate and concentrated under reduced pressure afforded 65 as a brown solid (400 mg; Yield: 86%). ¹H NMR (400 MHz, DMSO-d₆): δ 10.24 (s, 1H), 8.17 (s, 1H), 8.09 (s, 1H), 7.55 (s, 1H), 5.12 (m, 2H). LCMS: [M+H]⁺ 194.38, 87.09%.

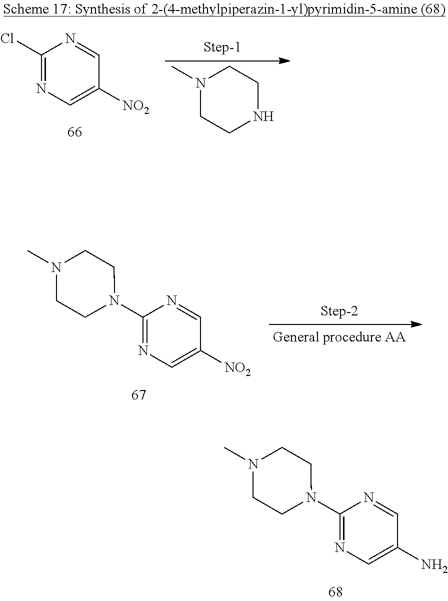

### Step 1: Preparation of 2-(4-methylpiperazin-1-yl)-5-nitropyrimidine (67)

To an ice-cold solution of 1-methylpiperazine (1.88 g, 18.8 mmol) in tetrahydrofuran (75 mL) was added triethylamine (5.24 mL, 37.6 mmol). The resulting mixture was stirred at same temperature for 15 minutes, followed by the addition of 2-chloro-5-nitropyrimidine (66) (3.0 g, 18.8 mmol) and stirred at room temperature for 12 hours. After completion of reaction (TLC monitoring), the reaction mixture was poured into water and extracted with ethyl acetate (3×100 mL). The combined organic layers were dried over anhydrous sodium sulfate and concentrated under reduced pressure afforded 67 as a yellow solid (3.6 g; Yield 83%). LCMS: [M+H]⁺ 224.02; 96.28%.

### Step 2: Preparation of 2-(4-methylpiperazin-1-yl)pyrimidin-5-amine (68)

Title compound was prepared in a manner substantially similar to procedure mentioned in General procedure AA. The crude was washed with ether afforded 68 as a brown color solid (1.5 g; Yield: 46%). LCMS: [M+H]⁺ 194.06; 99.6%

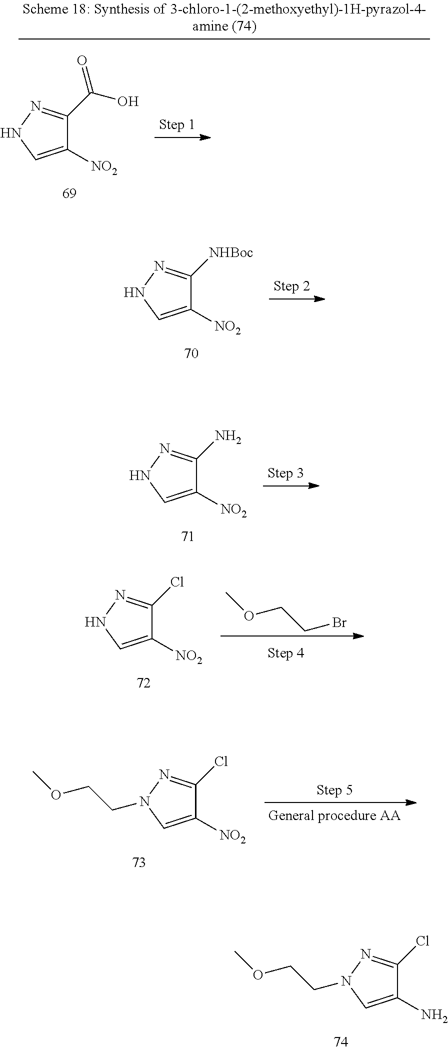

### Step 1: Preparation of tert-butyl (4-nitro-1H-pyrazol-3-yl)carbamate (70)

To an ice-cold solution of 4-nitro-1H-pyrazole-3-carboxylic acid (69) (2.5 g, 15.9 mmol) in toluene (50 mL) was added triethylamine (5.6 mL, 39.75 mmol) and diphenylphosphoryl azide (4.25 g, 17.5 mmol). The reaction mixture was stirred at room temperature for 6 hours. After completion of reaction (TLC monitoring), tert-butanol (25 mL) was added and heated at 130° C. for 16 hours. After completion of reaction, the reaction mixture was cooled to 0° C., quenched with water (100 mL) and extracted with ethyl acetate (3×100 mL). The combined organic layers were dried over anhydrous sodium sulfate, filtered and concentrated under reduced pressure. The crude was purified over combiflash, eluted with 30% ethyl acetate in heptane afforded the desired product (70) (600 mg, Yield: 16%). ¹H NMR (400 MHz, DMSO-d₆): δ 13.58 (bs, 1H), 9.33 (s, 1H), 8.44 (s, 1H), 1.44 (s, 9H).

### Step 2: Preparation of 4-nitro-1H-pyrazol-3-amine (71)

An ice-cold solution of tert-butyl (4-nitro-1H-pyrazol-3-yl)carbamate (70) (500 mg, 21.9 mmol) in 4M hydrochloric acid in dioxane (5 mL) was stirred at room temperature 6 hours. After completion of reaction (TLC monitoring), solvent was evaporated under reduced pressure to get desired product (71) (287 mg, Yield: 80%) as off white solid. MS: [M+H]⁺ 129.10

### Step 3: Preparation of 3-chloro-4-nitro-1H-pyrazole (72)

To an ice-cold solution of 4-nitro-1H-pyrazol-3-amine (71) (500 mg, 3.9 mmol) in hydrochloric acid (5.0 mL) was added aqueous solution of sodium nitrite (547 mg, 7.8 mmol) in water (1.0 mL). The resulting reaction mixture stirred at same temperature for 1 hour, followed by addition of copper(I) chloride (773 mg, 7.8 mmol) and stirred at room temperature for 16 hours. After completion of reaction (TLC monitoring), reaction mixture was diluted with ice-cold water (50 mL) and extracted with dichloromethane (3×50 mL). The combined organic layers were dried over anhydrous sodium sulfate, filtered and concentrated under reduced pressure afforded the desired product (72) (250 mg, Yield: 43%). MS: [M−H]⁺ 146.10

### Step 4: Preparation of 3-chloro-1-(2-methoxyethyl)-4-nitro-1H-pyrazole (73)

To an ice-cold solution of 3-chloro-4-nitro-1H-pyrazole (72) (300 mg, 2.03 mmol) in dimethylformamide (5.0 mL) was added sodium hydride (60% dispersion in mineral oil, 73 mg, 3.05 mmol) in portion wise. The resulting reaction mixture was stirred at the same temperature for 15 minutes, and 1-bromo-2-methoxyethane (73 mg, 2.03 mmol) was added. The reaction mixture was heated at 100° C. for 16 hours. After completion of reaction (as per TLC monitoring), the reaction mixture was poured into ice-cold water (50 mL) and extracted with ethyl acetate (3×50 mL). The combined organic layers were washed brine, dried over anhydrous sodium sulfate, filtered and concentrated under reduced pressure. The crude was purified over combiflash, eluted with 35% ethyl acetate in heptane afforded the desired product (73) (250 mg, Yield: 60%). ¹H-NMR (400 MHz, DMSO-d₆): δ 9.08 (bs, 1H), 4.86 (m, 2H), 4.74 (m, 2H), 3.24 (s, 3H).

### Step 5: Preparation of 3-chloro-1-(2-methoxyethyl)-1H-pyrazol-4-amine (74)

Title compound (74) was prepared (170 mg; Yield: 70%) as a viscous solid in a manner substantially similar to procedure mentioned in General procedure AA. MS: [M+H]⁺ 176.09

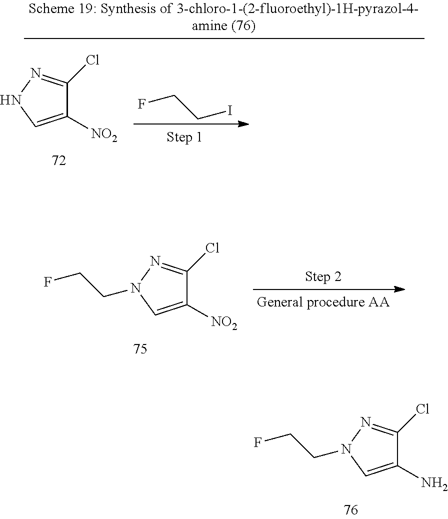

### Step 1: Preparation of 3-chloro-1-(2-fluoroethyl)-4-nitro-1H-pyrazole (75)

To an ice-cold solution of 3-chloro-4-nitro-1H-pyrazole (72) (250 mg, 1.69 mmol) in dimethylformamide (5.0 mL) was added sodium hydride (60% dispersion in mineral oil, 122 mg, 2.54 mmol) in portion wise. The resulting reaction mixture was stirred at same temperature for 15 minutes, and 1-bromo-2-fluoroethane (258 mg, 2.03 mmol) was added. The reaction mixture was heated at 100° C. for 16 hours. After completion of reaction (as per TLC monitoring), the reaction mixture was poured in to ice-cold water (50 mL) and extracted with ethyl acetate (3×50 mL). The combined organic layers were washed brine, dried over anhydrous sodium sulfate, filtered and concentrated under reduced pressure. The crude was purified over combiflash, eluted with 15% ethyl acetate in heptane afforded the desired product (75) (250 mg, Yield: 76%). ¹H-NMR (400 MHz, DMSO-d₆): δ 8.99 (s, 1H), 4.31 (m, 2H), 3.70 (s, 2H).

### Step 2: Preparation of 3-chloro-1-(2-fluoroethyl)-1H-pyrazol-4-amine (76)

Title compound (76) was prepared (200 mg; Yield: 95%) as viscous solid in a manner substantially similar to procedure mentioned in General procedure AA. MS: [M+H]⁺ 164.10

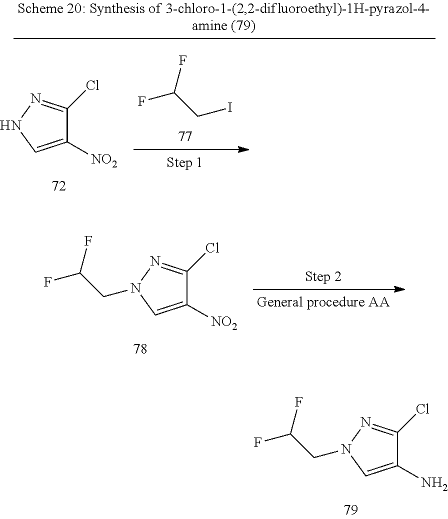 [SMILES: O=[N+]([O-])c1c[nH]nc1Cl ; FC(F)CI ; O=[N+]([O-])c1cn(CC(F)F)nc1Cl ; Nc1cn(CC(F)F)nc1Cl]

### Step 1: Preparation of 3-chloro-1-(2,2-difluoroethyl)-4-nitro-1H-pyrazole (78)

To an ice-cold solution of 3-chloro-4-nitro-1H-pyrazole (72) (300 mg, 2.04 mmol) in dimethylformamide (5.0 mL) was added sodium hydride (60% dispersion in mineral oil, 122 mg, 3.06 mmol) portion wise. The resulting reaction mixture was stirred at same temperature for 15 minutes, followed by the addition of 1-bromo-2-fluoroethane (77) (258 mg, 2.03 mmol). The resulting reaction mixture was heated at 100° C. for 16 hours. After completion of reaction (as per TLC monitoring), the reaction mixture was poured in to ice-cold water (50 mL) and extracted with ethyl acetate (3×50 mL). The combined organic layers were washed brine, dried over anhydrous sodium sulfate, filtered and concentrated under reduced pressure. The crude was purified over combiflash, eluted with 15% ethyl acetate in heptane afforded the desired product (78) (180 mg, Yield: 50%). ¹H-NMR (400 MHz, DMSO-d₆): δ 9.06 (s, 1H), 6.31 (m, 1H), 4.68 (s, 2H).

### Step 2: Preparation of 3-chloro-1-(2,2-difluoroethyl)-1H-pyrazol-4-amine (79)

Title compound (79) was prepared (120 mg; Yield: 78%) as viscous solid in a manner substantially similar to procedure mentioned in General procedure AA. MS: [M+H]⁺ 182.04

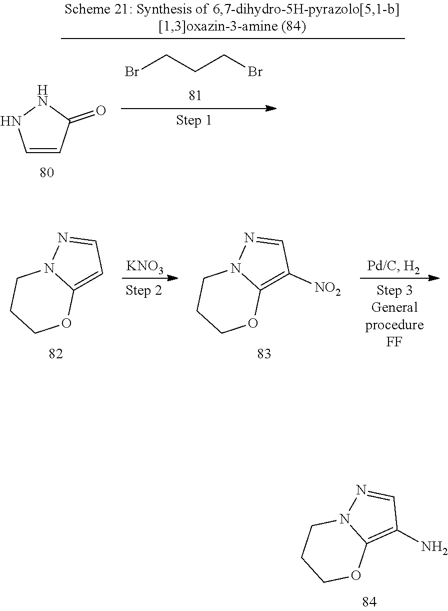

### Step 1: Preparation of 6,7-dihydro-5H-pyrazolo[5,1-b][1,3]oxazine (82)

To a solution of 1,2-dihydro-3H-pyrazol-3-one (80) (2.0 g, 23.8 mmol) in dimethylformamide (10 mL) was added potassium carbonate (2.96 g, 21.4 mmol) and 1,3-dibromopropane (81) (1.44 g, 7.14 mmol). The resulting reaction mixture was heated at 130° C. for 2 hours. After completion of reaction (as per TLC monitoring), reaction mixture was cooled to room temperature, quenched with ice-cold water and extracted with dichloromethane (3×100 mL). The combined organic layers were washed with brine, dried over anhydrous sodium sulfate, filtered and concentrated under reduced pressure. The crude was purified over combiflash, eluted with 0.5% methanol in dichloromethane afforded the desired product (82) (900 mg; Yield: 30%). ¹H-NMR (400 MHz, DMSO-d₆): δ 7.19 (d, J=1.6 Hz, 1H), 5.42 (t, J=2.0 Hz, 1H), 4.24 (t, J=5.2 Hz, 2H), 4.03 (t, J=6.0 Hz, 2H), 2.13 (m, 2H). MS: [M+H]⁺ 125.02.

### Step 2: Preparation of 3-nitro-6,7-dihydro-5H-pyrazolo[5,1-b][1,3]oxazine (83)

To an ice-cold solution of 6,7-dihydro-5H-pyrazolo[5,1-b][1,3]oxazine (82) (900 mg, 7.25 mmol) in concentrated sulfuric acid (11 mL) was added potassium nitrate (1.46 g, 14.4 mmol) portion wise and stirred the reaction mixture at the same temperature for 1 hour. After completion of reaction (TLC monitoring), reaction mixture poured into crushed ice and extracted with ethyl acetate (3×100 mL). The combined organic layers were washed with brine, dried over anhydrous sodium sulfate, filtered and concentrated under reduced pressure afforded the desired product (83) (630 mg; Yield: 52%). ¹H-NMR (400 MHz, DMSO-d₆): δ 8.07 (s, 1H), 4.54 (t, J=5.2 Hz, 2H), 4.10 (t, J=6.0 Hz, 2H), 2.24 (m, 2H). LCMS: [M+H]⁺ 170.19, 97.58% purity.

### Step 3: Preparation of 6,7-dihydro-5H-pyrazolo[5,1-b][1,3]oxazin-3-amine (84)

Title compound (84) was prepared (500 mg; Yield: 96%) in a manner substantially similar to procedure mentioned in General procedure FF. MS [M+H]⁺ 140.02

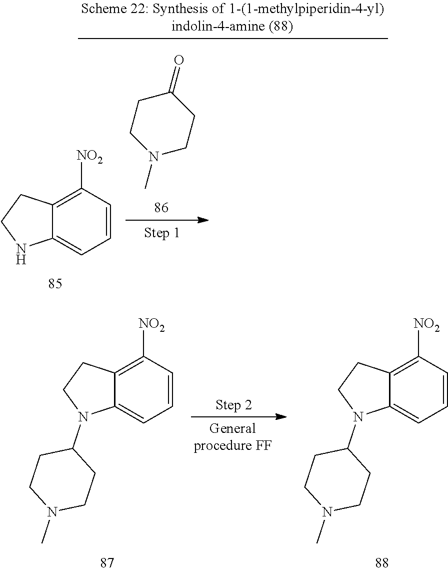

### Step 1: Preparation of 1-(1-methylpiperidin-4-yl)-4-nitroindoline (87)

To an ice-cold solution of 4-nitro-2,3-dihydro-1H-indole (85) (1.0 g, 6.09 mmol) in dichloroethane (20 mL) was added 1-methylpiperidine-4-one (86) (1.0 g, 8.83 mmol), catalytic amount of acetic acid and the reaction mixture was stirred for 10 minutes. Then the sodium triacetoxyborohydride (2.45 g, 11.6 mmol) was added to the above reaction mixture at 0° C. and reaction mixture was stirred at room temperature for 16 hours. After completion of reaction (TLC monitoring), the reaction mixture was quenched with saturated solution of sodium bicarbonate and extracted with dichloromethane (3×100 mL). The combined organic layers were washed with brine solution, dried over sodium sulfate, filtered, and concentrated under reduced pressure. The crude residue was purified by combiflash chromatography, eluted with 10% ethyl acetate in hexane afforded the desired product (87) (600 mg; Yield: 44%) as viscous liquid. MS [M+H]⁺ 262.25

### Step 2: Preparation of 1-(1-methylpiperidin-4-yl)indolin-4-amine (88)

Title compound (88) was prepared (400 mg; Yield: 82%) in a manner substantially similar to procedure mentioned in s General procedure FF. ¹H-NMR (400 MHz, DMSO-d₆): δ 6.64 (t, J=8.0 Hz, 1H), 5.86 (d, J=7.6 Hz, 1H), 5.71 (d, J=8.0 Hz, 1H), 4.62 (bs, 2H), 3.17 (m, 3H), 2.79 (m, 2H), 2.59 (m, 2H), 2.15 (s, 3H), 1.89 (m, 2H), 1.57 (m, 4H). LCMS [M+H]⁺ 232.24, 98.71%.

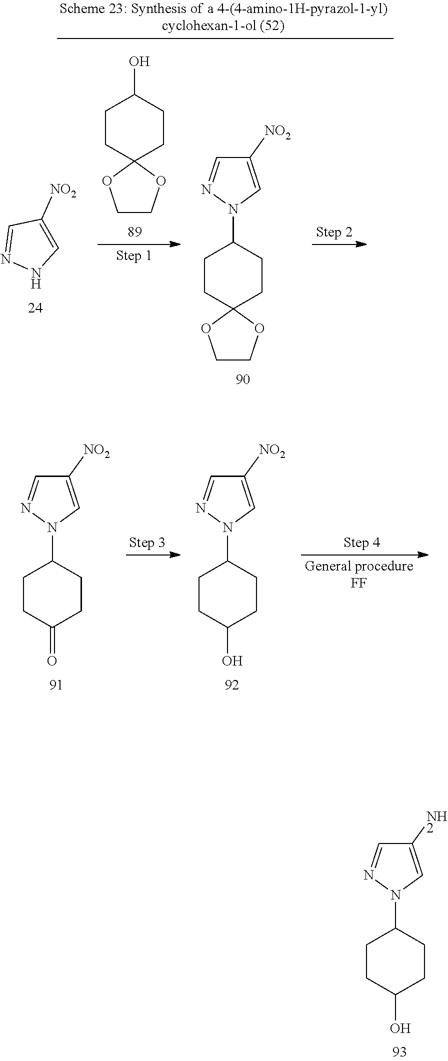

### Step 1: Preparation of 4-nitro-1-(1,4-dioxaspiro[4.5]decan-8-yl)-1H-pyrazole (90)

To an ice-cold solution of 4-nitro-1H-pyrazole (24) (5.0 g, 44.21 mmol) in tetrahydrofuran (75 mL) was added 1,4-dioxaspiro[4.5]decan-8-ol (89) (8.40 g, 53.05 mmol), triphenylphosphine (23.19 g, 88.42 mmol) and diisopropyl azodicarboxylate (17.87 g, 88.42 mmol). Then the reaction mixture was stirred at room temperature for 16 hours. After completion of reaction (TLC monitoring), the reaction mixture was poured into water and extracted with ethyl acetate (3×100 mL). The combined organic layers were washed with brine, dried over anhydrous sodium sulfate, filtered and concentrated under reduced pressure. The crude was purified over combiflash chromatography using 20% ethyl acetate in hexane as an eluent, afforded the 4-nitro-1-(1,4-dioxaspiro[4.5]decan-8-yl)-1H-pyrazole (90) as off white solid (6.0 g; Yield: 54%). ¹H-NMR (400 MHz, DMSO-d₆): δ 8.90 (s, 1H), 8.24 (s, 1H), 4.40-4.33 (m, 1H), 3.89 (s, 4H), 2.08-1.95 (m, 4H), 1.83-1.62 (m, 4H).

### Step 2: Preparation of 4-(4-nitro-1H-pyrazol-1-yl)cyclohexan-1-one (91)

To a solution of 4-nitro-1-(1,4-dioxaspiro[4.5]decan-8-yl)-1H-pyrazole (90) (6.0 g, 23.69 mmol) in acetone:water (1:1, 100 mL) was added pyridinium p-toluenesulfonate (17.86 g, 71.07 mmol). Then the reaction mixture was heated at 60° C. for 16 hours. After completion of reaction (TLC monitoring), solvent was evaporated, diluted with saturated solution of sodium bicarbonate and extracted with ethyl acetate (3×100 mL). The combined organic layer was washed with brine, dried over anhydrous sodium sulfate, filtered and concentrated under reduced pressure. The crude was purified using combiflash chromatography using (10-20%) ethyl acetate in hexane as an eluent, desired fractions were concentrated under reduced pressure afforded 4-(4-nitro-1H-pyrazol-1-yl)cyclohexan-1-one (91) as off white solid (4.0 g; Yield: 81%). ¹H-NMR (400 MHz, DMSO-d₆): δ 8.98 (s, 1H), 8.27 (s, 1H), 4.40-4.33 (m, 1H), 2.35-2.27 (m, 4H), 1.83-1.62 (m, 4H).

### Step 3: Preparation of 4-(4-nitro-1H-pyrazol-1-yl)cyclohexan-1-ol (92)

To an ice cold solution of 4-(4-nitro-1H-pyrazol-1-yl) cyclohexan-1-one (91) (1.0 g, 19.11 mmol) in methanol (50 mL) was added sodium borohydride (1.45 g, 38.23 mmol) portion-wise and the reaction mixture was stirred at room temperature for 4 hours. After completion of reaction (TLC monitoring), reaction mixture was quenched with ice-cold water and extracted with ethyl acetate (3×50 mL). The combined organic layers were washed with brine, dried over anhydrous sodium sulfate, filtered and concentrated under reduced pressure afforded 4-(4-nitro-1H-pyrazol-1-yl)cyclohexan-1-ol (92) as an off white solid (3.5 g; Yield: 86%). ¹H-NMR (400 MHz, DMSO-d₆): δ 8.90 (s, 1H), 8.25 (s, 1H), 4.30-4.19 (m, 1H), 3.50-3.44 (m, 1H), 2.28-1.95 (m, 4H), 1.85-1.70 (m, 5H).

### Step 4: Preparation of 4-(4-amino-1H-pyrazol-1-yl)cyclohexan-1-ol (93)

Title compound was prepared in a manner substantially similar to procedure mentioned in General procedure FF, afforded of 4-(4-amino-1H-pyrazol-1-yl)cyclohexan-1-ol (93) as a brown solid (2.5 g, Yield: 84%). ¹H-NMR (400 MHz, DMSO-d₆): δ 7.05 (s, 1H), 6.86 (s, 1H), 4.62-4.61 (m, 1H), 3.93-3.84 (m, 1H), 3.72 (s, 2H), 3.50-3.42 (m, 1H), 1.89-1.80 (m, 4H), 1.78-1.59 (m, 4H).

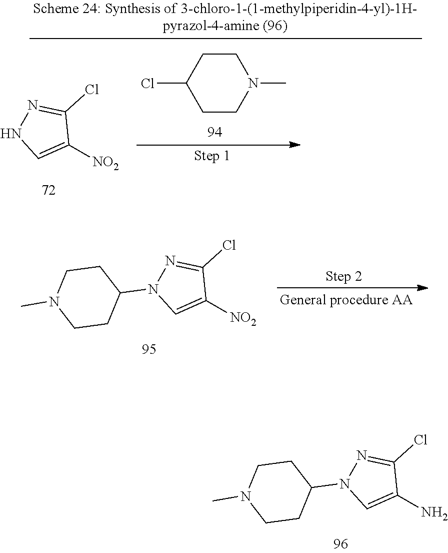

### Step 1: Preparation of 4-(3-chloro-4-nitro-1H-pyrazol-1-yl)-1-methylpiperidine (95)

To a solution of 3-chloro-4-nitro-1H-pyrazole (72) (8.0 g, 0.054 mol) in dimethylformamide (100 mL) was added cesium carbonate (35.44 g, 0.108 mol) and 4-chloro-1-methylpiperidine (94) (10.90 g, 0.081 mol) and the reaction mixture was heated at 120° C. for 16 hours. After completion of reaction (TLC monitoring), reaction mixture was diluted with ice-cold water and extracted with 10% methanol in dichloromethane (3×100 mL). The combined organic layer was washed with brine, dried over anhydrous sodium sulfate, filtered and concentrated under reduced pressure. The crude was purified by combiflash chromatography using 5-10% methanol in dichloromethane as eluent, desired fractions were concentrated to dryness under reduced pressure afforded 4-(3-chloro-4-nitro-1H-pyrazol-1-yl)-1-methylpiperidine (95) as a brown solid (5.0 g, Yield: 38%). ¹H-NMR (400 MHz, DMSO-d₆): δ 9.05 (s, 1H), 4.25-4.18 (m, 1H), 2.90-2.80 (m, 2H), 2.21 (s, 3H), 2.10-1.85 (m, 6H).

### Step 2: Preparation of 3-chloro-1-(1-methylpiperidin-4-yl)-1H-pyrazol-4-amine (96)

Title compound (96) was prepared as a brown solid (3.5 g; Yield: 80%) in a manner substantially similar to procedure mentioned in General procedure AA. ¹H-NMR (400 MHz, DMSO-d₆): δ 7.17 (s, 1H), 4.16 (m, 1H), 3.91-3.85 (m, 2H), 2.15 (s, 3H), 1.89-1.80 (m, 4H), 2.10-1.85 (m, 4H). MS: [M+H]⁺ 215.21

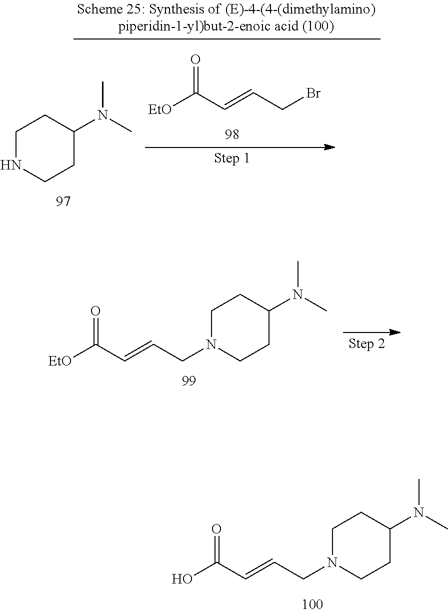

### Step 1: Preparation of ethyl (E)-4-(4-(dimethyl amino) piperidin-1-yl)but-2-enoate (99)

To a stirred solution N,N-dimethylpiperidin-4-amine hydrochloride (97) (500 mg, 3.04 mmol) in dichloromethane (3.00 mL) were added N,N-diisopropylethylamine (2.65 mL, 15.2 mmol) and ethyl (2E)-4-bromobut-2-enoate (98) (0.461 mL, 3.34 mmol) and the reaction mixture was stirred at room temperature for 16 hours. Progress of the reaction was monitored by LCMS and TLC. After completion of reaction, the reaction mixture was diluted with water (5 mL) and extracted with dichloromethane (2×100 mL). The combined organic layer was washed with brine (5 mL), dried over anhydrous sodium sulfate, filtered and evaporated under reduced pressure. The crude product was purified by flash chromatography using combiflash purifier with 6% methanol in dichloromethane as eluent to obtain ethyl (2E)-4-[4-(dimethylamino)piperidin-1-yl]but-2-enoate (99) (270 mg, 37% yield) as brown color liquid. LCMS [M+H]⁺ 241.2

### Step 2: Preparation of (E)-4-(4-(dimethyl amino) piperidin-1-yl)but-2-enoicacid (100)

To a stirred solution of ethyl (2E)-4-[4-(dimethyl amino)piperidin-1-yl]but-2-enoate (99) (270 mg, 1.25 mmol) in 1,4-dioxane (3 mL), was added hydrochloric acid (3 mL, 2N) and refluxed for 5 h. The progress of the reaction was monitored by LCMS and TLC. After completion of the reaction, reaction mixture was concentrated. The residue was washed with ether and concentrated to obtain (E)-4-[4-(dimethyl amino)piperidin-1-yl]but-2-enoic acid (100) (250 mg, crude) as white solid. LCMS [M+H]⁺ 213.2.

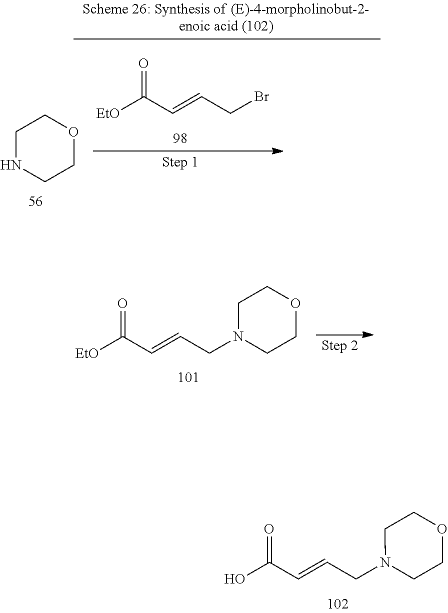

### Step 1: Synthesis of ethyl (2E)-4-(morpholin-4-yl)but-2-enoate (101)

To a stirred solution of morpholine (56) (0.5 g, 5.74 mmol) in dichloromethane (10.0 mL) were added ethylbis(propan-2-yl)amine (1.50 mL, 8.61 mmol), ethyl (2E)-4-bromobut-2-enoate (98) (1.22 g, 6.31 mmol) and stirred at room temperature for 16 hours. The progress of the reaction was monitored by LCMS. After completion of the reaction, reaction mixture was diluted with water, extracted with dichloromethane (50 mL×2). The combined organic layers were concentrated to afford title compound (101) (1.00 g, crude). LCMS [M+H]⁺ 200.1

### Step 2: Synthesis of (2E)-4-(morpholin-4-yl)but-2-enoic acid (102)

To a stirred solution of ethyl (2E)-4-(morpholin-4-yl)but-2-enoate (101) (1.00 g, 5.02 mmol) in 1,4-dioxane (10.0 mL), was added hydrochloric acid (10.0 mL, 2N) and refluxed for 3 hours. The progress of the reaction was monitored by LCMS. After completion of the reaction, reaction mixture was extracted with ethyl acetate (50 ml) and the organic layer was concentrated to obtain crude product. Residue was triturated ethyl acetate to afford title crude compound (102) (0.9 g, 90%) as light brown solid. LCMS [M+H]⁺ 172.1.

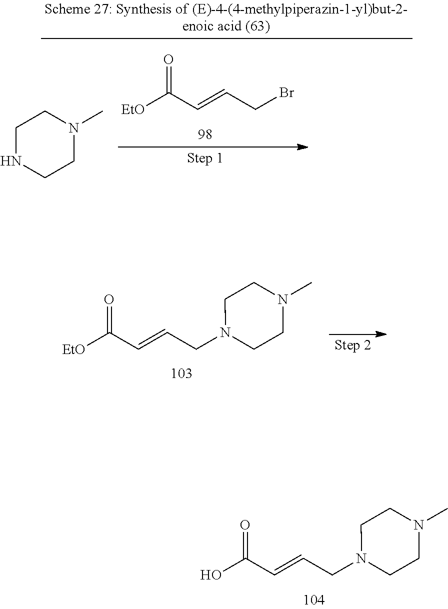

### Step 1: Synthesis of ethyl (2E)-4-(4-methylpiperazin-1-yl)but-2-enoate (103)

To a stirred solution of 1-methylpiperazine (0.5 g, 4.99 mmol) in dichloromethane (10.0 mL) were added ethylbis(propan-2-yl)amine (1.30 mL, 7.49 mmol), ethyl (2E)-4-bromobut-2-enoate (98) (1.06 g, 5.49 mmol) and stirred at room temperature for 16 h. The progress of the reaction was monitored by LCMS. After completion of the reaction, reaction mixture was diluted with water (20 ml) and extracted with ethyl acetate (100 ml×2). The combined organic layers were concentrated to obtain title compound (103) (0.66 g, crude). LCMS [M+H]⁺ 213.2.

### Step 2: Synthesis of (2E)-4-(4-methylpiperazin-1-yl)but-2-enoic acid (104)

To a stirred solution of ethyl (2E)-4-(4-methylpiperazin-1-yl)but-2-enoate (103) (0.66 g, 3.11 mmol) in 1,4-dioxane (10.0 mL), was added hydrochloric acid (10.0 mL, 2N) and refluxed for 3 hours. The progress of the reaction was monitored by LCMS. After completion of the reaction, reaction mixture was extracted with ethyl acetate (50 ml) and the organic layer was concentrated to obtain crude product. Residue was triturated with ether to obtain title compound (104) as light brown solid (0.6 g, crude). LCMS [M+H]⁺ 185.1.

### Compound 1: N-(3-(3-benzyl-7-((1-methyl-1H-pyrazol-4-yl)amino)-2-oxo-3,4-dihydropyrimido[4,5-d]pyrimidin-1(2H)-yl)phenyl)acrylamide

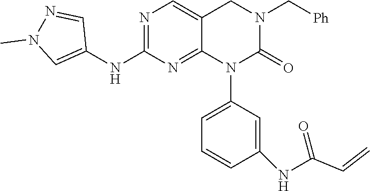 [SMILES: C=CC(=O)Nc1cccc(N2C(=O)N(Cc3ccccc3)Cc3cnc(Nc4cnn(C)c4)nc32)c1]

### Step 1: Ethyl 4-((3-((tert-butoxycarbonyl)amino)phenyl)amino)-2-(methylthio)pyrimidine-5-carboxylate

To a solution of 4-chloro-2-methylsulfanyl-pyrimidine-5-carboxylic acid ethyl ester (2, 10.84 g, 46.58 mmol) in DMF (100 mL) were added potassium carbonate (12.87 g, 93.16 mmol) and tert-butyl (3-aminophenyl)carbamate (9.69 g, 46.58 mmol). The reaction was heated to 80° C. and stirred for 16 h. The reaction was monitored by TLC, after the completion of reaction, the reaction mixture was cooled to room temperature and the reaction mixture was added to ice water. The precipitate was filtered and the filtered cake was rinsed with additional cold water and dried in vacuum to give ethyl 4-((3-((tert-butoxycarbonyl)amino)phenyl)amino)-2-(methylthio)pyrimidine-5-carboxylate (15.5 g, 82.4% yield). LCMS Calcd for [M+H]⁺ 405.1, found 405.2

### Step 2: tert-butyl (3-((5-(hydroxymethyl)-2-(methylthio)pyrimidin-4-yl)amino)phenyl)carbamate

To a solution of the ethyl 4-((3-((tert-butoxycarbonyl) amino)phenyl)amino)-2-methylthio)pyrimidine-5-carboxylate (13 g, 32.17 mmol) in THE (200 mL) was added 1.0 M LiAlH₄ solution in THF (3.65 g, 96.5 mmol) at −40° C. The temperature was raised to RT for a period of 3 h. The reaction mixture then treated with ammonium chloride solution (50 mL). After the mixture was stirred at RT for 30 min, the solid was filtered off. The filtrate was partitioned between DCM (5×200 mL) and water (200 mL). The organic layer was washed with brine (200 mL), dried over anhydrous sodium sulfate and evaporated under reduced pressure to obtain crude product. The crude product was purified by column chromatography using combiflash with 0-5% methanol in DCM as a solvent to get pure product tert-butyl (3-((5-(hydroxymethyl)-2-(methylthio)pyrimidin-4-yl)amino)phenyl)carbamate (4.3 g, 37% yield). LCMS Calcd for [M+H]⁺ 363.1, LCMS found 363.2

### Step 3: tert-butyl (3-((5-formyl-2-(methylthio)pyrimidin-4-yl)amino)phenyl)carbamate

To a solution of tert-butyl (3-((5-(hydroxymethyl)-2-(methylthio)pyrimidin-4-yl)amino)phenyl)carbamate (3.5 g, 9.66 mmol) in DCM (40 mL) was added activated manganese oxide (8.4 g, 96.6 mmol) at RT and the reaction mixture was stirred for 16 h. The reaction was monitored by TLC, after the completion of reaction, the reaction mixture was filtered and the filtrate was concentrated under reduced pressure to obtain crude product tert-butyl (3-((5-formyl-2-(methylthio)pyrimidin-4-yl)amino)phenyl)carbamate (2.8 g, 80.4% yield) and used as such for the next step. LCMS Calcd for [M+H]⁺ 361.1, LCMS found 361.2

### Step 4: (E)-tert-butyl (3-((5-((benzylimino)methyl)-2-(methylthio)pyrimidin-4-yl)amino)phenyl)carbamate

Title compound was prepared in a similar manner to general procedure A. tert-butyl (3-((5-formyl-2-(methylthio)pyrimidin-4-yl)amino)phenyl)carbamate and benzyl amine gave title compound (E)-tert-butyl (3-((5-((benzylimino)methyl)-2-(methylthio)pyrimidin-4-yl)amino)phenyl)carbamate (3.1 g, 88.8% yield). LCMS Calcd for [M+H]⁺ 450.1, LCMS found 450.2

### Step 5: tert-butyl (3-((5-((benzylamino)methyl)-2-(methylthio)pyrimidin-4-yl)amino)phenyl)carbamate

Title compound was prepared in a similar manner to general procedure B. (E)-tert-butyl (3-((5-((benzylimino)methyl)-2-(methylthio)pyrimidin-4-yl)amino)phenyl)carbamate gave tert-butyl (3-((5-((benzylamino)methyl)-2-(methylthio)pyrimidin-4-yl)amino)phenyl)carbamate with 87% yield. LCMS Calcd for [M+H]⁺ 452.2, LCMS found 452.3

### Step 6: tert-butyl (3-(3-benzyl-7-(methylthio)-2-oxo-3,4-dihydropyrimido[4,5-d]pyrimidin-1(2H)-yl)phenyl)carbamate

Title compound was prepared in a similar manner to general procedure C. tert-butyl (3-((5-((benzylamino) methyl)-2-(methylthio)pyrimidin-4-yl)amino)phenyl)carbamate gave tert-butyl (3-(3-benzyl-7-(methylthio)-2-oxo-3,4-dihydropyrimido[4,5-d]pyrimidin-1(2H)-yl)phenyl)carbamate with 70% yield. LCMS Calcd for [M+H]⁺ 478.1, LCMS found 478.2

### Step 7: tert-butyl (3-(3-benzyl-7-(methylsulfonyl)-2-oxo-3,4-dihydropyrimido[4,5-d]pyrimidin-1(2H)-yl)phenyl)carbamate

Title compound was prepared in a similar manner to general procedure D. tert-butyl (3-(3-benzyl-7-(methylthio)-2-oxo-3,4-dihydropyrimido[4,5-d]pyrimidin-1(2H)-yl)phenyl)carbamate gave tert-butyl (3-(3-benzyl-7-(methylsulfonyl)-2-oxo-3, 4-dihydropyrimido[4,5-d]pyrimidin-1(2H)-yl)phenyl)carbamate with 99% yield. ¹H NMR (400 MHz, CDCl₃): δ 8.39 (s, 1H), 7.62 (s, 1H), 7.41-7.35 (m, 6H), 7.18-7.16 (d, 1H), 7.65 (s, 1H), 7.51-7.50 (t, 1H), 7.37-7.35 (m, 5H), 7.06-7.04 (d, 2H), 6.62 (s, 1H), 6.45-6.38 (m, 1H), 6.26-6.21 (m, 1H), 5.76-5.73 (d, 1H), 4.62 (s, 2H), 4.37 (s, 2H), 3.47 (s, 3H);

### Step 8: tert-butyl (3-(3-benzyl-7-((1-methyl-1H-pyrazol-4-yl)amino)-2-oxo-3,4-dihydropyrimido[4,5-d]pyrimidin-1(2H)-yl)phenyl)carbamate

Title compound was prepared in a similar manner to general procedure E. tert-butyl (3-(3-benzyl-7-(methylsulfonyl)-2-oxo-3,4-dihydropyrimido[4,5-d]pyrimidin-1(2H)-yl)phenyl)carbamate and 1-methyl-1H-pyrazol-4-amine gave tert-butyl (3-(3-benzyl-7-((1-methyl-1H-pyrazol-4-yl)amino)-2-oxo-3,4-dihydropyrimido[4,5-d]pyrimidin-1(2H)-yl)phenyl)carbamate as off white solid with 8% yield. LCMS Calcd for [M+H]⁺ 527.2, found 527.5

### Step 9: 1-(3-aminophenyl)-3-benzyl-7-((1-methyl-1H-pyrazol-4-yl)amino)-3,4-dihydropyrimido[4,5-d]pyrimidin-2(1H)-one

Title compound was prepared in a similar to general procedure F. tert-butyl (3-(3-benzyl-7-((1-methyl-1H-pyrazol-4-yl)amino)-2-oxo-3,4-dihydropyrimido[4,5-d]pyrimidin-1(2H)-yl)phenyl)carbamate and TFA and DCM as solvent gave 1-(3-aminophenyl)-3-benzyl-7-((1-methyl-1H-pyrazol-4-yl)amino)-3,4-dihydropyrimido[4,5-d]pyrimidin-2(1H)-one as off white solid with 80 yield. LCMS cd for [M+H]⁺ 427.1, found 427.5

### Step 10: N-(3-(3-benzyl-7-((1-methyl-1H-pyrazol-4-yl)amino)-2-oxo-3,4-dihydropyrimido[4,5-d]pyrimidin-1(28)-yl)phenyl)acrylamide (Compound 1)

Title compound was prepared in a similar manner to general procedure G. 1-(3-aminophenyl)-3-benzyl-7-((1-methyl-1H-pyrazol-4-yl)amino)-3,4-dihydropyrimido[4, 5-d]pyrimidin-2(1H)-one and acryloyl chloride gave N-(3-(3-benzyl-7-((1-methyl-1H-pyrazol-4-yl)amino)-2-oxo-3,4-dihydropyrimido[4,5-d]pyrimidin-1(2H)-yl)phenyl)acrylamide (Compound 1) as white solid with 2900 yield. ¹H NMR (400 MHz), DMSO-d₆): δ 10.30 (s, 1H), 9.33 (s, 1H), 8.06 (s, 1H), 7.79 (bs, 1H), 7.65 (s, 1H), 7.50 (bs, 1H), 7.37-7.35 (m, 5H), 7.05 (d, J=8 Hz, 2H), 6.62 (s, 1H), 6.45-6.38 (m, 1H), 6.26-6.21 (m, 1H), 5.76-5.73 (m, 1H), 4.62 (s, 2H), 4.37 (s, 2H), 3.47 (s, 3H); LCMS Calcd for [M+H]⁺ 481.2, found 481.5

In some cases, for example compound 38 and compound 39, the racemic compounds (compound 37) were separated by chiral prep HPLC using Chiral Cel-OJH (20×250) mm, 5μ column (mobile phase:Hexane-EtOH) with a flow rate of 18 mL/min to get pure enantiomers.

The following compounds were prepared using the methods described above

| | | Synthe- | | |
|---|---|---|---|---|
| Cmpd | | sis | LCMS | ¹H-NMR (400 MHz, |
| No. | Structure | method | [M + H]⁺ | DMSO-d₆) |
| 2 | 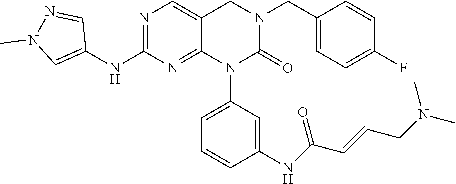 | H | 556.25 | δ 10.26 (s, 1H), 9.38 (s, 1H), 8.09 (s, 1H), 7.78-7.70 (m, 1H), 7.68 (s, 1H), 7.51-7.49 (m, 1H), 7.44-7.40 (m, 2H), 7.21 (t, J = 8.8 Hz, 2H), 7.05-7.03 (m, 2H), 6.76-6.72 (m, 1H), 6.69 (s, 1H), 6.26 (d, J = 15.6 Hz, 1H), 4.62 (s, 2H), 4.39 (s, 2H), 3.48 (s, 3H), 3.05 (d, J = 5.2 Hz, 2H), 2.16 (s, 6H). |
| 3 | 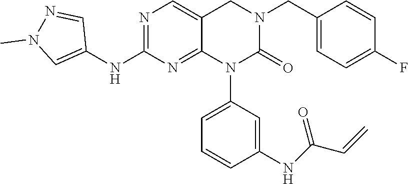 [SMILES: C=CC(=O)Nc1cccc(N2C(=O)N(Cc3ccc(F)cc3)Cc3cnc(Nc4cnn(C)c4)nc32)c1] | G | 499.46 | δ 10.36 (s, 1H), 9.39 (s, 1H), 8.09 (s, 1H), 7.81-7.79 (m, 1H), 7.68 (s, 1H), 7.59-7.51 (m, 1H), 7.44-7.40 (m, 2H), 7.21 (t, J = 8.8 Hz, 2H), 7.08-7.03 (m, 2H), 6.62 (s, 1H), 6.47-6.40 (m, 1H), 6.25 (d, J = 17.2 Hz, 1H), 5.77 (d, J = 9.6 Hz, 1H), 4.62 (s, 2H), 4.39 (s, 2H), 3.48 (s, 3H). |
| 4 | 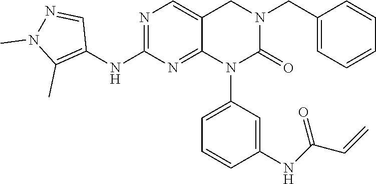 [SMILES: C=CC(=O)Nc1cccc(N2C(=O)N(Cc3ccccc3)Cc3cnc(Nc4cnn(C)c4C)nc32)c1] | G | 493.44 | δ 10.25 (s, 1H), 8.62 (bs, 1H), 8.02 (s, 1H), 7.65-7.63 (m, 2H), 7.40-7.24 (m, 7H), 6.98 (d, J = 6.8 Hz, 1H), 6.47-6.40 (m, 1H), 6.25 (d, J = 16.8 Hz, 1H), 5.75 (d, J = 10.4 Hz, 1H), 4.62 (s, 2H), 4.36 (s, 2H), 3.55 (s, 3H), 2.00 (s, 3H). |
| 5 | 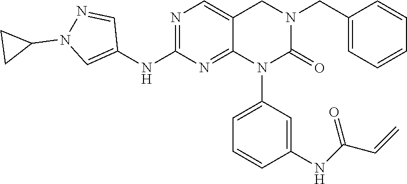 [SMILES: C=CC(=O)Nc1cccc(N2C(=O)N(Cc3ccccc3)Cc3cnc(Nc4cnn(C5CC5)c4)nc32)c1] | G | 507.24 | δ 10.33 (s, 1H), 9.38 (s, 1H), 8.09 (s, 1H), 7.82 (d, J = 8.8 Hz, 1H), 7.70 (s, 1H), 7.54-7.52 (m, 1H), 7.41-7.31 (m, 4H), 7.31- 7.30 (m, 1H), 7.08-7.03 (m, 2H), 6.76 (s, 1H), 6.47-6.41 (m, 1H), 6.25 (d, J = 15.2 Hz, 1H), 5.76 (d, J = 10.4 Hz, 1H), 4.64 (s, 2H), 4.39 (s, 2H), 3.32-3.30 (m, 1H), 0.86-0.83 (m, 2H), 0.73-0.71 (m, 2H). |
| 6 | 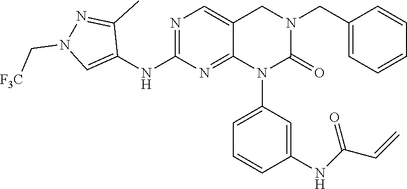 [SMILES: C=CC(=O)Nc1cccc(N2C(=O)N(Cc3ccccc3)Cc3cnc(Nc4cn(CC(F)(F)F)nc4C)nc32)c1] | G | 563.48 | δ 10.34 (s, 1H), 8.97 (s, 1H), 8.13 (s, 1H), 7.79 (d, J = 7.6 Hz, 1H), 7.63 (s, 1H), 7.49-7.47 (m, 1H), 7.45-7.37 (m, 4H), 7.33- 7.31 (m, 1H), 7.06-7.04 (m, 1H), 6.70 (s, 1H), 6.45-6.38 (m, 1H), 6.25 (d, J = 15.6 Hz, 1H), 5.78-5.75 (m, 1H), 4.6 (s, 2H), 4.41 (s, 2H), 4.43-4.41 (m, 2H), 2.09 (s, 3H). |
| 7 | 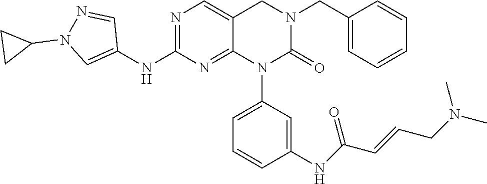 [SMILES: CN(C)C/C=C/C(=O)Nc1cccc(N2C(=O)N(Cc3ccccc3)Cc3cnc(Nc4cnn(C5CC5)c4)nc32)c1] | H | 564.55 | δ 10.25 (s, 1H), 9.55 (s, 1H), 9.38 (s, 1H), 8.09 (s, 1H), 7.83 (s, 1H), 7.78 (s, 1H), 7.50 (s, 1H), 7.37-7.32 (m, 5H), 7.03 (s, 1H), 6.75-6.70 (m, 2H), 6.27 (d, J = 14.4 Hz, 1H), 4.63 (s, 2H), 4.39 (s, 2H), 3.05 (s, 2H), 2.16 (s, 6H), 1.17 (s, 1H), 0.85 (s, 2H), 0.73 (s, 2H). |
| 8 | 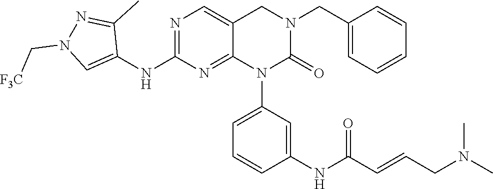 [SMILES: Cc1nn(CC(F)(F)F)cc1Nc1ncc2c(n1)N(c1cccc(NC(=O)/C=C/CN(C)C)c1)C(=O)N(Cc1ccccc1)C2] | H | 620.54 | δ 10.24 (s, 1H), 8.97 (bs, 1H), 8.13 (s, 1H), 7.76 (d, J = 5.2 Hz, 1H), 7.64 (s, 1H), 7.47-7.31 (m, 6H), 7.02 (d, J = 6.4 Hz, 1H), 6.75-6.70 (m, 2H), 6.24 (d, J = 17.2 Hz, 1H) 4.63 (s, 2H), 4.57- 4.41 (m, 2H), 4.41 (s, 2H), 3.04 (d, J = 5.2 Hz, 2H), 2.16 (s, 6H), 2.09 (s, 3H). |
| 9 | 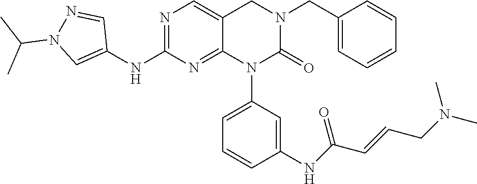 [SMILES: CC(C)n1cc(Nc2ncc3c(n2)N(c2cccc(NC(=O)/C=C/CN(C)C)c2)C(=O)N(Cc2ccccc2)C3)cn1] | H | 566.29 | δ 10.39 (s, 1H), 9.37 (s, 1H), 8.09 (s, 1H), 7.83-7.81 (m, 1H), 7.68 (s, 1H), 7.48 (t, J = 7.6 Hz, 1H), 7.41-7.29 (m, 5H), 7.05- 7.03 (m, 2H), 6.76-6.69 (m, 2H), 6.26 (d, J = 15.2 Hz, 1H), 4.63 (s, 2H), 4.45 (s, 2H), 4.01- 3.99 (m, 1H), 3.04 (d, J = 5.6 Hz, 2H), 2.15 (s, 6H), 1.21 (d, J = 20.8 Hz, 6H). |
| 10 | 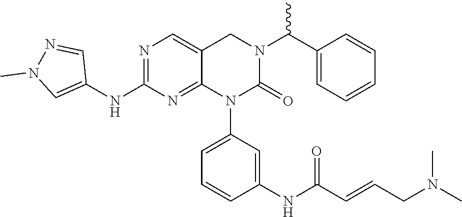 | H | 552.61 | δ 10.27 (s, 1H), 9.37 (s, 1H), 8.06 (s, 1H), 7.78-7.76 (m, 1H), 7.69 (s, 1H), 7.49 (t, J = 6.8 Hz, 1H), 7.41-7.40 (m, 4H), 7.33- 7.30 (m, 1H), 7.06-7.04 (m, 2H), 6.76-6.71 (m, 1H), 6.69 (s, 1H), 6.27 (d, J = 15.6 Hz, 1H), 5.77-5.72 (m, 1H), 4.43 (d, J = 14 Hz, 1H), 3.93 (d, J = 14 Hz, 1H), 3.48 (s, 3H), 3.07 (d, J = 5.6 Hz, 2H), 2.18 (s, 6H), 1.60 (d, J = 7.2 Hz, 3H). |
| 11 | 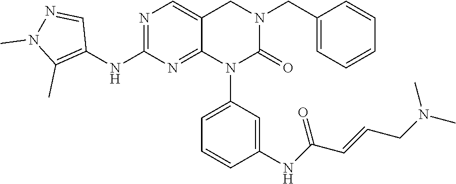 | H | 552.56 | δ 10.17 (s, 1H), 8.57 (bs, 1H), 8.02 (s, 1H), 7.64-7.62 (m, 2H), 7.39-7.31 (m, 7H), 6.95 (d, J = 8 Hz, 1H), 6.76-6.69 (m, 1H), 6.26 (d, J = 15.2 Hz, 1H), 4.61 (s, 2H), 4.36 (s, 2H), 3.55 (s, 3H), 3.05 (d, J = 4.2 Hz, 2H), 2.17 (s, 6H), 2.00 (s, 3H). |
| 12 | 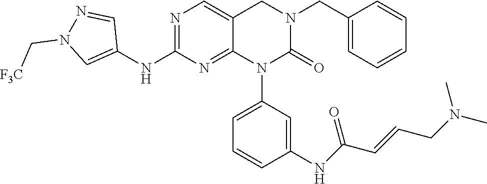 [SMILES: CN(C)C/C=C/C(=O)Nc1cccc(N2C(=O)N(Cc3ccccc3)Cc3cnc(Nc4cnn(CC(F)(F)F)c4)nc32)c1] | H | 606.52 | δ 10.29 (s, 1H), 9.51 (s, 1H), 8.12 (s, 1H), 7.89-7.77 (m, 1H), 7.66 (s, 1H), 7.49-7.41 (m, 1H), 7.41-7.37 (m, 4H), 7.35-7.31 (m, 1H), 7.20 (bs, 1H), 7.05 (d, J = 7.6 Hz, 1H), 6.77-6.69 (m, 2H), 6.28 (d, J = 15.2 Hz, 1H), 4.64 (s, 2H), 4.62-4.55 (m, 2H), 4.40 (s, 2H), 3.18 (bs, 2H), 2.25 (s, 6H). |
| 13 | 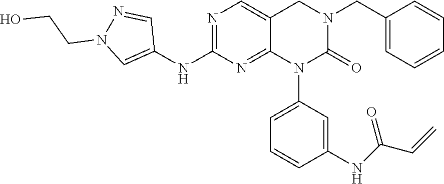 [SMILES: C=CC(=O)Nc1cccc(N2C(=O)N(Cc3ccccc3)Cc3cnc(Nc4cnn(CCO)c4)nc32)c1] | G | 511.49 | δ 10.39 (s, 1H), 9.38 (s, 1H), 8.09 (s, 1H), 7.78-7.76 (m, 1H), 7.72 (s, 1H), 7.50 (bs, 1H), 7.41-7.31 (m, 6H), 7.08-7.04 (m, 2H), 6.71 (bs, 1H), 6.49- 6.42 (m, 1H), 6.27-6.22 (m, 1H), 5.77-5.74 (m, 1H), 4.71 (s, 1H), 4.64 (s, 1H), 4.39 (s, 2H), 3.79-3.71 (m, 2H), 3.53-3.49 (m, 2H). |
| 14 | 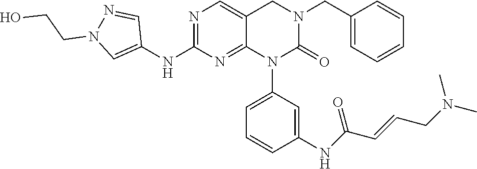 [SMILES: CN(C)C/C=C/C(=O)Nc1cccc(N2C(=O)N(Cc3ccccc3)Cc3cnc(Nc4cnn(CCO)c4)nc32)c1] | H | 568.51 | δ 10.31 (s, 1H), 9.38 (s, 1H), 8.09 (s, 1H), 7.75-7.71 (m, 2H), 7.52-7.48 (m, 1H), 7.42-7.29 (m, 5H), 7.06-7.04 (m, 2H), 6.76-6.70 (m, 2H), 6.30 (d, J = 15.2 Hz, 1H), 4.72 (bs, 1H), 4.63 (s, 2H), 4.39 (s, 2H), 3.76 (bs, 2H), 3.52 (bs, 2H), 3.20 (bs, 2H), 2.29 (s, 6H). |
| 15 | 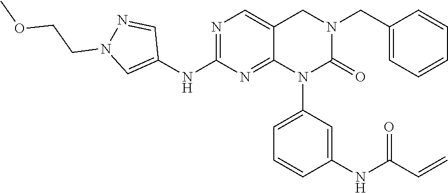 [SMILES: C=CC(=O)Nc1cccc(N2C(=O)N(Cc3ccccc3)Cc3cnc(Nc4cnn(CCOC)c4)nc32)c1] | G | 525.49 | δ 10.34 (s, 1H), 9.39 (s, 1H), 8.09 (s, 1H), 7.80-7.73 (m, 1H), 7.67 (s, 1H), 7.52-7.48 (m, 1H), 7.45-7.30 (m, 5H), 7.07-7.05 (m, 2H), 6.69 (s, 1H), 6.47-6.40 (m, 1H), 6.25 (d, J = 15.6 Hz, 1H), 5.76 (d, J = 11.6 Hz, 1H), 4.64 (s, 2H), 4.40 (s, 2H), 3.85 (bs, 2H), 3.44 (bs, 2H), 3.13 (s, 3H). |
| 16 | 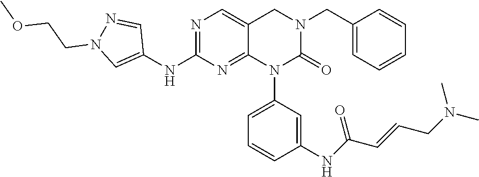 | H | 582.55 | δ 10.25 (s, 1H), 9.39 (s, 1H), 8.09 (s, 1H), 7.84-7.78 (m, 1H), 7.68 (s, 1H), 7.54-7.45 (m, 1H), 7.45-7.31 (m, 5H), 7.08-7.03 (m, 2H), 6.76-6.69 (m, 2H), 6.26 (d, J = 15.6 Hz, 1H), 4.63 (s, 2H), 4.39 (s, 2H), 3.85 (bs, 2H), 3.45 (bs, 2H), 3.13 (s, 3H), 3.04 (d, J = 6.0 Hz, 2H), 2.16 (s, 6H). |
| 17 | 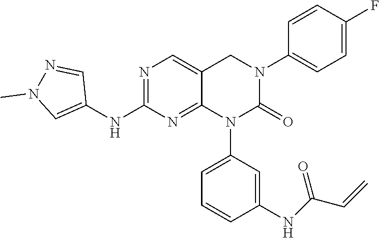 [SMILES: C=CC(=O)Nc1cccc(N2C(=O)N(c3ccc(F)cc3)Cc3cnc(Nc4cnn(C)c4)nc32)c1] | G | 483.44 | δ 10.41 (s, 1H), 9.46 (s, 1H), 8.17 (s, 1H), 7.79-7.77 (m, 1H), 7.76 (s, 1H), 7.52-7.49 (m, 3H), 7.26 (t, J = 8.8 Hz, 2H), 7.10 (d, J = 8 Hz, 1H), 7.05 (s, 1H), 6.66 (s, 1H), 6.49-6.42 (m, 1H), 6.28-6.23 (m, 1H), 5.78-5.75 (m, 1H), 4.85 (s, 2H), 3.49 (s, 3H). |
| 18 | 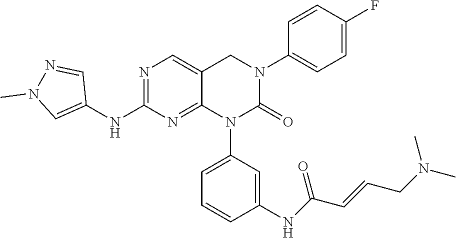 | H | 542.5 | δ 10.32 (s, 1H), 9.45 (s, 1H), 8.17 (s, 1H), 7.77-7.73 (m, 2H), 7.55-7.45 (m, 3H), 7.26 (t, J = 8.4 Hz, 2H), 7.10-7.04 (m, 2H), 6.75-6.66 (m, 2H), 6.29 (d, J = 15.6 Hz, 1H), 4.85 (s, 2H), 3.49 (s, 3H), 3.16 (bs, 2H), 2.24 (s, 6H). |
| 19 | 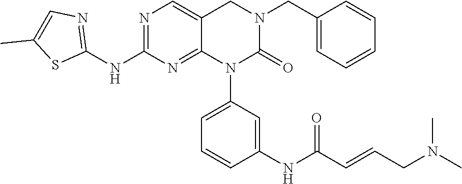 | H | 555.30 | δ 11.18 (s, 1H), 10.22 (s, 1H), 8.23 (s, 1H), 7.77 (d, J = 7.6 Hz, 1H), 7.68 (s, 1H), 7.46-7.32 (m, 6H), 7.03 (d, J = 7.2 Hz, 1H), 6.87 (s, 1H), 6.75-6.68 (m, 1H), 6.27 (d, J = 15.2 Hz, 1H), 4.64 (s, 2H), 4.45 (s, 2H), 3.05 (d, J = 4.8 Hz, 2H), 2.17 (s, 6H), 2.09 (s, 3H). |
| 20 | 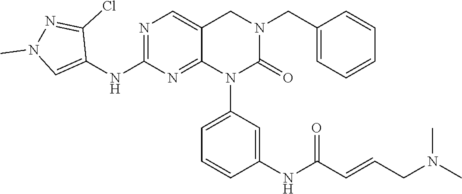 | H | 572.52 | δ 10.26 (s, 1H), 8.77 (bs, 1H), 8.12 (s, 1H), 7.76-7.74 (m, 1H), 7.65 (s, 1H), 7.51-7.47 (m, 1H), 7.41 (s, 5H), 7.33-7.31 (m, 1H), 7.04 (d, J = 8 Hz, 1H), 6.77- 6.70 (m, 1H), 6.26 (d, J = 15.6 Hz, 1H), 4.63 (s, 2H), 4.41 (s, 2H), 3.48 (bs, 3H), 3.04 (d, J = 5.6 Hz, 2H), 2.16 (s, 6H). |
| 21 | 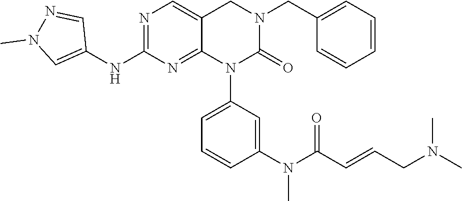 | H | 552.63 | δ 9.45 (bs, 1H), 8.27 (s, 1H), 7.72-7.65 (m, 1H), 7.54-7.47 (m, 1H), 7.37 (s, 5H), 7.32-7.27 (m, 2H), 7.02 (s, 1H), 6.74-6.68 (m, 1H), 6.64-6.56 (m, 1H), 6.00-5.90 (m, 1H), 4.64 (s, 2H), 4.37 (s, 2H), 3.51 (s, 3H), 3.27 (s, 3H), 2.43 (bs, 2H), 1.90 (s, 6H). |
| 22 | 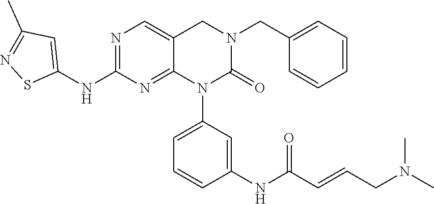 [SMILES: Cc1cc(Nc2ncc3c(n2)N(c2cccc(NC(=O)/C=C/CN(C)C)c2)C(=O)N(Cc2ccccc2)C3)sn1] | H | 555.57 | δ 11.13 (bs, 1H), 10.21 (s, 1H), 8.26 (bs, 1H), 7.80 (bs, 1H), 7.67-7.65 (m, 1H), 7.44-7.32 (m, 6H), 7.04 (bs, 1H), 6.74- 6.63 (m, 1H), 6.56-6.46 (m, 1H), 6.27 (d, J = 14.8 Hz, 1H), 4.78 (s, 2H), 4.45 (s, 2H), 3.04 (bs, 2H), 2.16 (s, 9H). |
| 23 | 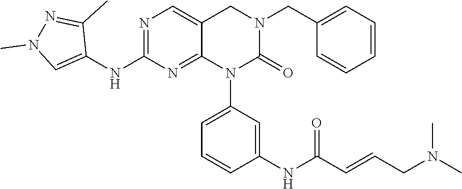 | H | 552.60 | δ 10.28 (s, 1H), 8.83 (bs, 1H), 8.10 (s, 1H), 7.79-7.77 (m, 1H), 7.65 (s, 1H), 7.51-7.43 (m, 1H), 7.37 (s, 4H), 7.33-7.31 (m, 1H), 7.04 (d, J = 7.6 Hz, 1H), 6.76- 6.70 (m, 1H), 6.58-6.55 (m, 1H), 6.28 (d, J = 16 Hz, 1H), 4.63 (s, 2H), 4.40 (s, 2H), 3.39 (s, 3H), 3.10 (bs, 2H), 2.20 (s, 6H), 2.04 (s, 3H). |
| 24 | 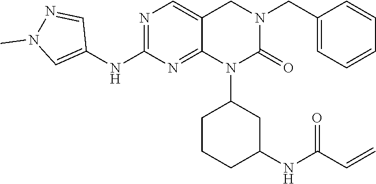 [SMILES: C=CC(=O)NC1CCCC(N2C(=O)N(Cc3ccccc3)Cc3cnc(Nc4cnn(C)c4)nc32)C1] | G | 487.58 | δ 9.41-9.29 (m, 1H), 8.15-8.13 (m, 1H), 8.05 (s, 1H), 7.84-7.82 (m, 1H), 7.49 (s, 1H), 7.36-7.22 (m, 5H), 6.23-6.16 (m, 1H), 6.06 (d, J = 16.8 Hz, 1H), 5.57- 5.54 (m, 1H), 4.75 (bs, 1H), 4.63-4.49 (m, 2H), 4.17 (s, 2H), 3.82 (bs, 4H), 2.40-2.33 (m, 2H), 1.90-1.82 (m, 3H), 1.62- 1.58 (m, 1H), 1.40-1.28 (m, 1H), 1.16-1.09 (m, 1H). |
| 25 | 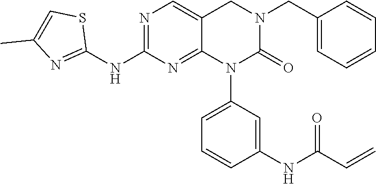 [SMILES: C=CC(=O)Nc1cccc(N2C(=O)N(Cc3ccccc3)Cc3cnc(Nc4nc(C)cs4)nc32)c1] | G | 498.49 | δ 11.28 (bs, 1H), 10.26 (bs, 1H), 8.25 (s, 1H), 7.71-7.65 (m, 2H), 7.38-7.33 (m, 6H), 7.07 (bs, 1H), 6.42-6.40 (m, 1H), 6.26 (bs, 2H), 5.76 (bs, 1H), 4.64 (s, 2H), 4.46 (s, 2H), 2.12 (s, 3H). |
| 26 | 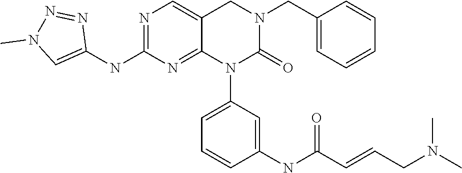 | H | 539.22 | δ 10.29 (s, 1H), 10.16 (s, 1H), 8.15 (s, 1H), 7.81 (d, J = 8 Hz, 1H), 7.66 (s, 1H), 7.54 (t, J = 8 Hz, 1H), 7.38 (s, 4H), 7.33-7.31 (m, 1H), 7.07 (d, J = 7.2 Hz, 1H), 6.77-6.70 (m, 1H), 6.44 (bs, 1H), 6.27 (d, J = 15.2 Hz, 1H), 4.64 (s, 2H), 4.43 (s, 2H), 3.71 (s, 3H), 3.07 (bs, 2H), 2.18 (s, 6H). |
| 27 | 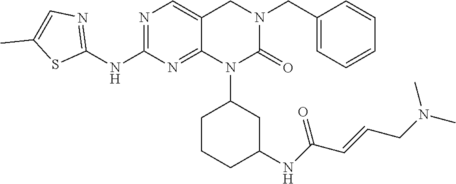 | H | 561.25 | δ 11.37 (s, 1H), 8.15 (s, 1H), 8.04 (d, J = 7.2 Hz, 1H), 7.38- 7.27 (m, 5H), 7.08 (s, 1H), 6.56- 6.49 (m, 1H), 6.00 (d, J = 16 Hz, 1H), 4.89 (s, 1H), 4.64-4.51 (m, 2H), 4.23 (s, 2H), 3.83 (s, 1H), 3.01 (s, 2H), 2.46-2.37 (m, 2H), 2.33 (s, 3H), 2.16 (s, 6H), 1.99-1.76 (m, 3H), 1.76-1.71 (m, 1H), 1.43-1.29 (m, 1H), 1.23-1.12 (m, 1H). |
| 28 | 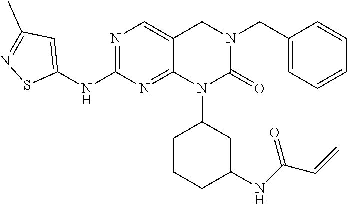 [SMILES: C=CC(=O)NC1CCCC(N2C(=O)N(Cc3ccccc3)Cc3cnc(Nc4cc(C)ns4)nc32)C1] | G | 504.21 | δ 11.35 (bs, 1H), 8.19-8.14 (m, 2H), 7.38-7.29 (m, 5H), 6.66 (s, 1H), 6.22-6.16 (m, 1H), 6.12- 6.03 (m, 1H), 5.55 (d, J = 10 Hz, 1H), 5.0-4.9 (m, 1H), 4.66- 4.49 (m, 2H), 4.24 (s, 2H), 3.33 (bs, 1H), 2.29 (s, 3H), 1.87-1.67 (m, 5H), 1.38-1.33 (m, 3H). |
| 29 | 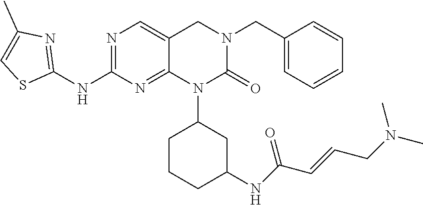 [SMILES: Cc1csc(Nc2ncc3c(n2)N(C2CCCC(NC(=O)/C=C/CN(C)C)C2)C(=O)N(Cc2ccccc2)C3)n1] | H | 561.25 | δ 11.47 (s, 1H), 8.17 (s, 1H), 8.11 (bs, 1H), 7.38-7.28 (m, 5H), 6.68 (s, 1H), 6.58-6.51 (m, 1H), 6.06 (d, J = 15.6 Hz, 1H), 4.86 (bs, 1H), 4.63-4.51 (m, 2H), 4.23 (s, 2H), 3.83 (bs, 1H), 3.24 (bs, 2H), 2.32 (s, 6H), 2.25 (s, 3H), 1.99-1.69 (m, 4H), 1.46-1.41 (m, 1H), 1.29-1.18 (m, 3H). |
| 30 | 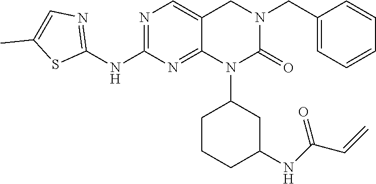 [SMILES: C=CC(=O)NC1CCCC(N2C(=O)N(Cc3ccccc3)Cc3cnc(Nc4ncc(C)s4)nc32)C1] | G | 504.18 | δ 11.36 (s, 1H), 8.16 (s, 1H), 8.12 (d, J = 7.6 Hz, 1H), 7.39- 7.35 (m, 2H), 7.31-7.29 (m, 3H), 7.07 (s, 1H), 6.22-6.16 (m, 1H), 6.08-6.04 (m, 1H), 5.56- 5.53 (m, 1H), 4.95-4.85 (m, 1H), 4.64-4.51 (m, 2H), 4.24 (s, 2H), 3.92-3.83 (m, 1H), 2.36 (s, 3H), 2.45-2.36 (m, 2H), 1.93- 1.82 (m, 3H), 1.74-1.71 (m, 1H), 1.44-1.41 (m, 1H), 1.25- |
| | | | | 1.21 (m, 1H). |
| 31 | 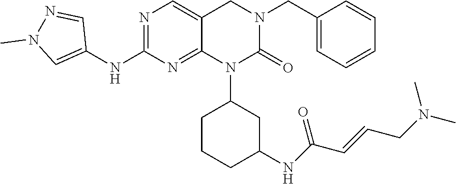 | H | 544.31 | δ 9.31 (s, 1H), 8.05-8.00 (m, 2H), 7.81 (bs, 1H), 7.44 (s, 1H), 7.36-7.28 (m, 5H), 6.56-6.51 (m, 1H), 6.00 (d, J = 16 Hz, 1H), 4.74 (bs, 1H), 4.62-4.49 (m, 2H), 4.16 (s, 2H), 3.81 (s, 3H), 3.77 (bs, 1H), 2.95 (d, J = 6 Hz, 2H), 2.42-2.33 (m, 2H), 2.12 (s, 6H), 1.9-1.8 (m, 3H), 1.67-1.66 (m, 1H), 1.39-1.36 (m, 1H), 1.23-1.17 (m, 1H). |
| 32 | 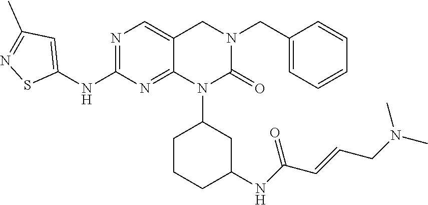 [SMILES: Cc1cc(Nc2ncc3c(n2)N(C2CCCC(NC(=O)/C=C/CN(C)C)C2)C(=O)N(Cc2ccccc2)C3)sn1] | H | 561.25 | δ 11.25 (bs, 1H), 8.12 (s, 1H), 8.05 (d, J = 8 Hz, 1H), 7.38- 7.29 (m, 5H), 6.66 (s, 1H), 6.56-6.50 (m, 1H), 6.00 (d, J = 15.2 Hz, 1H), 4.95-4.83 (m, 1H), 4.65-4.50 (m, 2H), 4.23 (s, 2H), 3.78 (bs, 1H), 2.97 (d, J = 4.8 Hz, 2H), 2.87 (s, 3H), 2.14 (s, 6H), 1.89- 1.69 (m, 4H), 1.54-1.49 (m, 1H), 1.23-1.09 (m, 3H). |
| 33 | 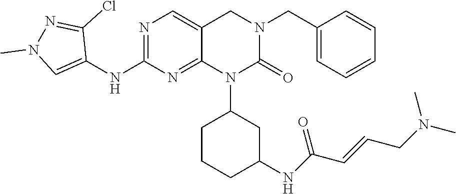 | H | 580.22 [M + 2H]⁺ | δ 8.61 (s, 1H), 7.97 (bs, 2H), 7.89 (s, 1H), 7.37-7.34 (m, 2H), 7.29-7.27 (m, 3H), 6.57-6.50 (m, 1H), 6.05-6.01 (m, 1H), 4.70-4.41 (m, 3H), 4.16 (s, 2H), 3.80 (s, 3H), 3.64 (bs, 1H), 3.03 (bs, 2H), 2.33-2.32 (m, 2H), 2.17 (s, 6H), 1.80-1.77 (m, 3H), 1.58-1.56 (m, 1H), 1.35-1.30 (m, 1H), 1.03 (bs, 1H). |
| 34 | 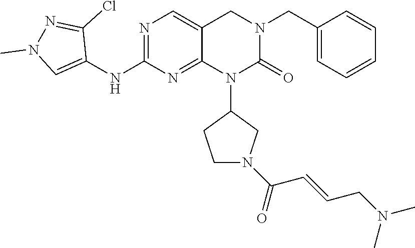 | H | 552.18 [M + 2H]⁺ | δ 8.59 (bs, 1H), 8.02-8.01 (m, 1H), 7.82-7.79 (m, 1H), 7.38- 7.34 (m, 2H), 7.29 (d, J = 7.6 Hz, 3H), 6.66-6.61 (m, 1H), 6.39-6.28 (m, 1H), 5.37 (bs, 1H), 4.59-4.57 (m, 2H), 4.19 (bs, 2H), 3.73-3.72 (m, 5H), 3.58-3.55 (m, 1H), 3.06-3.00 (m, 2H), 2.73 (bs, 1H), 2.14 (s, 6H), 1.99-1.97 (m, 2H). |
| 35 | 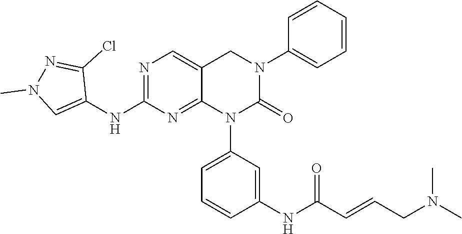 | H | 560.17 [M + 2H]⁺ | δ 10.27 (bs, 1H), 8.86 (bs, 1H), 8.21 (s, 1H), 7.73 (s, 2H), 7.48- 7.41 (m, 6H), 7.29-7.26 (m, 1H), 7.10-7.08 (m, 1H), 6.74- 6.66 (m, 1H), 6.27 (d, J = 15.6 Hz, 1H), 4.89 (s, 2H), 3.50 (bs, 3H), 3.05 (d, J = 5.6 Hz, 2H), 2.17 (s, 6H). |
| 36 | 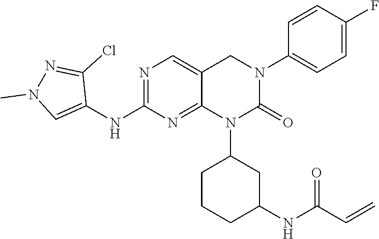 [SMILES: C=CC(=O)NC1CCCC(N2C(=O)N(c3ccc(F)cc3)Cc3cnc(Nc4cn(C)nc4Cl)nc32)C1] | G | 525.37 | δ 8.68 (bs, 1H), 8.06 (s, 1H), 8.04 (bs, 1H), 7.91 (s, 1H), 7.39-7.35 (m, 2H), 7.24-7.20 (m, 2H), 6.23-6.04 (m, 2H), 5.57-5.54 (m, 1H), 4.65-4.56 (m, 3H), 3.81 (s, 3H), 3.66 (bs, 1H), 2.32 (bs, 1H), 1.88-1.75 (m, 3H), 1.63 (bs, 1H), 1.30 (bs, 1H), 1.07 (bs, 2H). |
| 37 (Ra- ce- mic) | 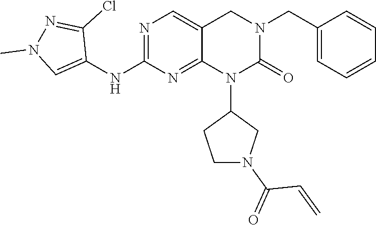 [SMILES: C=CC(=O)N1CCC(N2C(=O)N(Cc3ccccc3)Cc3cnc(Nc4cn(C)nc4Cl)nc32)C1] | G | 493.12 | δ 8.58 (bs, 1H), 8.01 (s, 1H), 7.82-7.79 (m, 1H), 7.37-7.29 (m, 5H), 6.66-6.47 (m, 1H), 6.14 (d, J = 16.4 Hz, 1H), 5.63- 5.40 (m, 1H), 5.38 (bs, 1H), 4.58 (s, 2H), 4.18 (s, 2H), 3.98- 3.93 (m, 1H), 3.73-3.54 (m, 6H), 2.72-2.64 (m, 1H), 2.10- 1.96 (m, 1H). |
| 38 (Enan- tio- mer 1) | 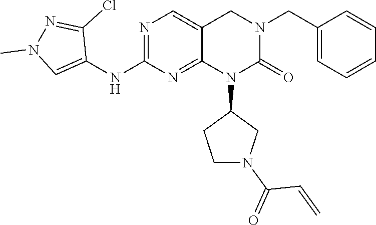 [SMILES: C=CC(=O)N1CC[C@@H](N2C(=O)N(Cc3ccccc3)Cc3cnc(Nc4cn(C)nc4Cl)nc32)C1] | G | 493.38 | δ 8.58 (bs, 1H), 8.01 (d, J = 3.60 Hz, 1H), 7.82-7.74 (m, 1H), 7.38-7.29 (m, 5H), 6.66- 6.47 (m, 1H), 6.14 (d, J = 16.80 Hz, 1H), 5.67-5.62 (m, 1H), 5.42 (bs, 1H), 4.53 (s, 2H), 4.18 (s, 2H), 3.98-3.93 (m, 1H), 3.73-3.50 (m, 6H), 2.69-2.61 (m, 1H), 2.10-1.80 (m, 1H). |
| 39 (Enan- tio- mer 2) | 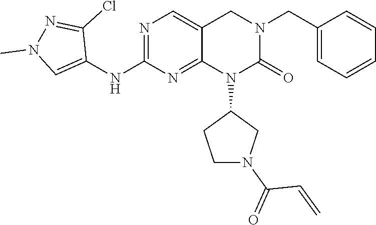 [SMILES: C=CC(=O)N1CC[C@H](N2C(=O)N(Cc3ccccc3)Cc3cnc(Nc4cn(C)nc4Cl)nc32)C1] | G | 493.38 | δ 8.60 (bs, 1H), 8.01 (d, J = 3.60 Hz, 1H), 7.82-7.74 (m, 1H), 7.38-7.29 (m, 5H), 6.66- 6.47 (m, 1H), 6.12 (d, J = 16.40 Hz, 1H), 5.70-5.63 (m, 1H), 5.38 (bs, 1H), 4.58 (s, 2H), 4.18 (s, 2H), 3.97-3.93 (m, 1H), 3.73-3.50 (m, 6H), 2.69-2.61 (m, 1H), 2.11-1.97 (m, 1H). |
| 40 | 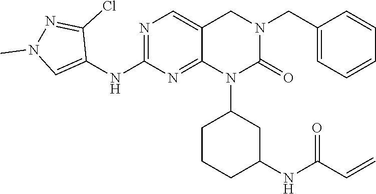 [SMILES: C=CC(=O)NC1CCCC(N2C(=O)N(Cc3ccccc3)Cc3cnc(Nc4cn(C)nc4Cl)nc32)C1] | G | 521.38 | δ 8.61 (bs, 1H), 8.02 (bs, 1H), 7.97 (s, 1H), 7.88 (s, 1H), 7.37- 7.27 (m, 5H), 6.23-6.16 (m, 1H), 6.09-6.04 (m, 1H), 5.55 (dd, J₁ = 2.0, J₂ = 10 Hz, 1H), 4.72-4.48 (m, 3H), 4.15 (s, 2H), 3.80 (s, 3H), 3.64 (bs, 1H), 3.38-3.32 (m, 2H), 1.81-1.75 (m, 3H), 1.56 (bs, 1H), 1.36- 1.02 (m, 2H). |
| 41 | 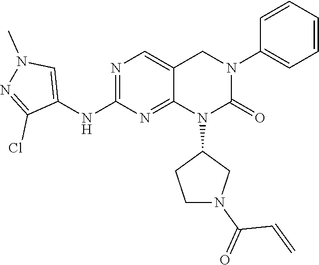 [SMILES: C=CC(=O)N1CC[C@H](N2C(=O)N(c3ccccc3)Cc3cnc(Nc4cn(C)nc4Cl)nc32)C1] | G | 479.33 | δ 8.68 (bs, 1H), 8.11 (d, J = 2.80 Hz, 1H), 7.90-7.80 (m, 1H), 7.42-7.35 (m, 4H), 7.27- 7.23 (m, 1H), 6.64-6.47 (m, 1H), 6.13 (d, J = 17.20 Hz, 1H), 5.69-5.63 (m, 1H), 5.36 (bs, 1H), 4.66 (s, 2H), 3.97 (t, J = 9.2 Hz, 1H), 3.74-3.73 (m, 5H), 3.57-3.55 (m, 1H), 2.67-2.57 (m, 1H), 2.13-1.99 (m, 1H). |
| 42 | 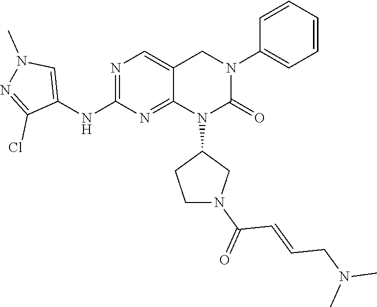 [SMILES: CN(C)C/C=C/C(=O)N1CC[C@H](N2C(=O)N(c3ccccc3)Cc3cnc(Nc4cn(C)nc4Cl)nc32)C1] | H | 536.40 | δ 8.68 (bs, 1H), 8.11-8.10 (m, 1H), 7.94-7.85 (m, 1H), 7.42- 7.34 (m, 4H), 7.27-7.23 (m, 1H), 6.68-6.58 (m, 1H), 6.42- 6.28 (m, 1H), 5.30 (bs, 1H), 4.66 (s, 2H), 3.99-3.94 (m, 1H), 3.80-3.65 (m, 5H), 3.60-3.50 (m, 1H), 3.05-3.02 (m, 2H), 2.72 (bs, 1H), 2.15 (s, 6H), 1.98-1.97 (m, 1H). |
| 43 | 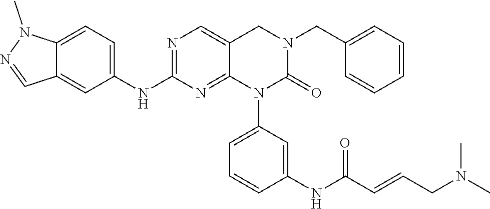 [SMILES: CN(C)C/C=C/C(=O)Nc1cccc(N2C(=O)N(Cc3ccccc3)Cc3cnc(Nc4ccc5c(cnn5C)c4)nc32)c1] | H | 588.50 | δ 10.35 (s, 1H), 9.46 (s, 1H), 8.16 (s, 1H), 7.86 (d, J = 7.6 Hz, 1H), 7.75 (s, 1H), 7.62-7.59 (m, 2H), 7.53-7.49 (m, 1H), 7.38 (bs, 4H), 7.34-7.25 (m, 3H), 7.07 (d, J = 7.2 Hz, 1H), 6.73-6.68 (m, 1H), 6.29 (d, J = 12.8 Hz, 1H), 4.65 (s, 2H), 4.43 (s, 2H), 3.92 (s, 3H), 3.19 (bs, 2H), 2.25 (s, 6H). |
| 44 | 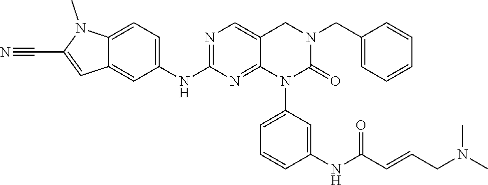 [SMILES: CN(C)C/C=C/C(=O)Nc1cccc(N2C(=O)N(Cc3ccccc3)Cc3cnc(Nc4ccc5c(c4)cc(C#N)n5C)nc32)c1] | H | 612.70 | δ 10.27 (s, 1H), 9.39 (s, 1H), 8.14 (s, 1H), 7.84 (d, J = 8.4 Hz, 1H), 7.71 (bs, 1H), 7.57 (bs, 1H), 7.50 (t, J = 8 Hz, 1H), 7.36 (bs, 4H), 7.29-7.26 (m, 3H), 7.04 (d, J = 7.2 Hz, 1H), 6.90 (s, 1H), 6.74-6.67 (m, 1H), 6.25 (d, J = 15.6 Hz, 1H), 4.63 (s, 2H), 4.41 (s, 2H), 3.76 (s, 3H), 3.09 (bs, 2H),2.19 (s, 6H). |
| 45 | 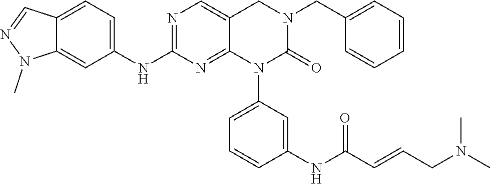 [SMILES: CN(C)C/C=C/C(=O)Nc1cccc(N2C(=O)N(Cc3ccccc3)Cc3cnc(Nc4ccc5cnn(C)c5c4)nc32)c1] | H | 588.59 | δ 10.26 (s, 1H), 9.58 (s, 1H), 8.21 (s, 1H), 7.79 (s, 1H), 7.72- 7.69 (m, 2H), 7.50-7.31 (m, 8H), 7.15 (d, J = 8.8 Hz, 1H), 7.08 (d, J = 8.4 Hz, 1H), 6.76- 6.69 (m, 1H), 6.28 (d, J = 15.2 Hz, 1H), 4.64 (s, 2H), 4.43 (s, 2H), 3.65 (s, 3H), 3.17 (bs, 2H), 2.25 (s, 6H). |
| 46 | 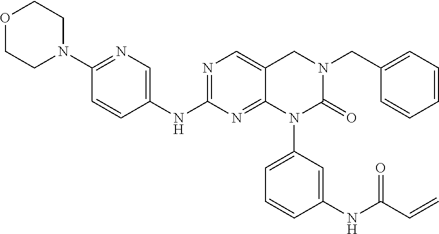 [SMILES: C=CC(=O)Nc1cccc(N2C(=O)N(Cc3ccccc3)Cc3cnc(Nc4ccc(N5CCOCC5)nc4)nc32)c1] | G | 563.8 | δ 10.26 (s, 1H), 9.15 (bs, 1H), 8.08 (s, 1H), 8.03 (bs, 1H), 7.78 (d, J = 8 Hz, 1H), 7.57 (s, 1H), 7.51 (bs, 1H), 7.44-7.29 (6H), 7.00 (d, J = 7.6 Hz, 1H), 6.45- 6.38 (m, 2H), 6.23 (d, J = 16.4 Hz, 1H), 5.74 (d, J = 10.8 Hz, 1H), 4.62 (s, 2H), 4.38 (s, 2H), 3.65 (s, 4H), 3.28 (s, 4H). |
| 47 | 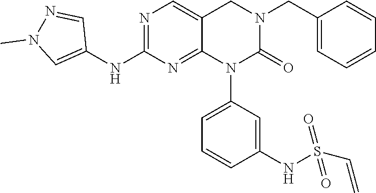 [SMILES: C=CS(=O)(=O)Nc1cccc(N2C(=O)N(Cc3ccccc3)Cc3cnc(Nc4cnn(C)c4)nc32)c1] | G | 517.2 | δ 10.19 (s, 1H), 9.37 (bs, 1H), 8.06 (s, 1H), 7.48 (bs, 1H), 7.37-7.35 (m, 4H), 7.29 (bs, 2H), 7.10-6.98 (m, 3H), 6.78- 6.68 (m, 1H), 6.60-6.50 (m, 1H), 6.10-6.04 (m, 1H), 5.88- 5.10 (m, 1H), 4.61 (s, 2H), 4.36 (s, 2H). 3H were merged with DMSO water peak. |
| 48 | 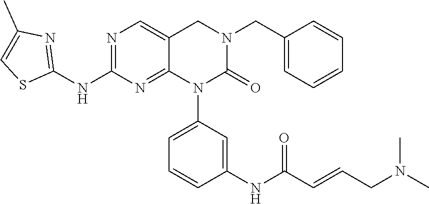 [SMILES: Cc1csc(Nc2ncc3c(n2)N(c2cccc(NC(=O)/C=C/CN(C)C)c2)C(=O)N(Cc2ccccc2)C3)n1] | H | 555.6 | δ 11.18 (bs, 1H), 10.12 (s, 1H), 8.21 (s, 1H), 7.67 (d, J = 8 Hz, 1H), 7.62 (s, 1H), 7.41-7.29 (m, 6H), 7.01 (d, J = 7.2 Hz, 1H), 6.74-6.67 (m, 1H), 6.25 (s, 1H), 6.22 (s, 1H), 4.62 (s, 2H), 4.43 (s, 2H), 3.02 (d, J = 5.2 Hz, 2H), 2.14 (s, 6H), 2.10 (s, 3H). |

### Compound 49: (E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-4-(dimethylamino)but-2-enamide

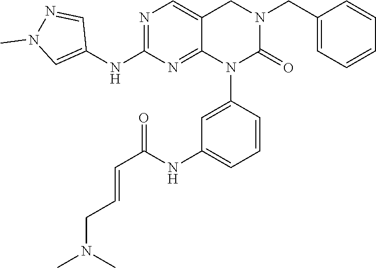 [SMILES: CN(C)C/C=C/C(=O)Nc1cccc(N2C(=O)N(Cc3ccccc3)Cc3cnc(Nc4cnn(C)c4)nc32)c1]

### Step 1: Synthesis of (E)-4-(dimethylamino)but-2-enoyl chloride

To a stirred mixture of acetonitrile (2 mL) and DMF (2 drop) under N₂ atmosphere was added N,N-dimethylamino crotonic acid hydrochloride (0.1 g, 0.77 mmol). After 10 min, this solution was cooled to 0-5° C. Oxalyl chloride (0.122 g, 0.968 mmol) was added and the reaction mixture was maintained at 0-5° C. for 30 min. It was allowed to warm to RT and stirring was continued for 2 h. It was then heated to 40° C. for 5 min and again brought to RT and stirred for 10 min. Formation of product was confirmed by TLC and the reaction mass was used as such to the next step without any workup.

### Step-2: Synthesis of (E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 49)

1-(3-Aminophenyl)-3-benzyl-7-((1-methyl-1H-pyrazol-4-yl)amino)-3,4-dihydropyrimido[4,5-d]pyrimidin-2(1H)-one (0.1 g, 0.7 mmol) in DMP (2 mL) was cooled to −15° C. and then (E)-4-(dimethylamino)but-2-enoylchloride was added. The reaction mixture was stirred for 1 h at −15° C. to RT. After the completion of reaction, the reaction mass was quenched with ice water, sodium bicarbonate solution and extracted with DCM (100 mL×2). The combined organic layer was washed with cold water (3×50 mL), brine solution (10 mL), dried over anhydrous sodium sulfate and evaporated under reduced pressure to obtain crude product. The crude product was purified by prep HPLC to get pure product (E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-4-(dimethylamino)but-2-enamide (Compound 49, 0.022 g, 16% yield) as white solid. ¹H NMR (400 MHz, DMSO-d₆): δ 10.21 (s, 1H), 9.32 (s, 1H), 8.06 (s, 1H), 7.76 (bs, 1H) 7.65 (s, 1H), 7.48 (bs, 1H), 7.39-7.29 (m, 5H), 7.03 (d, J=7.2 Hz, 2H), 6.74-6.68 (m, 1H), 6.62 (s, 1H), 6.25 (d, J=15.2 Hz, 1H), 4.62 (s, 2H), 4.37 (s, 2H), 3.47 (s, 3H), 3.03 (d, J=5.6 Hz, 2H), 2.15 (s, 6H); LCMS Calcd for [M+H]⁺ 538.2, found 538.5

### Compound 50: (E)-N-(3-(3-benzyl-7-((1-methyl-1H-pyrazol-4-yl)amino)-2-oxo-3,4-dihydropyrimido[4,5-d]pyrimidin-1(2H)-yl)phenyl)-3-chloroacrylamide

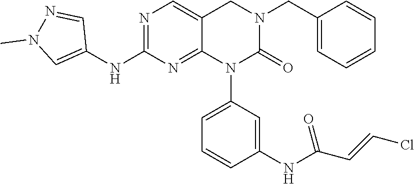 [SMILES: Cn1cc(Nc2ncc3c(n2)N(c2cccc(NC(=O)/C=C/Cl)c2)C(=O)N(Cc2ccccc2)C3)cn1]

### Step-1: Synthesis of (Z)-3-chloroacrylic acid

To a stirred solution propiolic acid (2 g, 28.5 mmol) in DMF (15 mL) under N₂ atmosphere was added thionyl chloride (4.07 g, 34.2 moles) slowly and the reaction mixture was maintained at 25° C. for 1 h. The reaction was monitored by TLC, after the completion of reaction, the residue was poured into ice and the resulting aqueous solution was extracted with ether (3×100 mL). The organic layer was washed with brine (20 mL), dried over anhydrous sodium sulfate and evaporated under reduced pressure to obtain crude product. The crude product was purified to get pure product (Z)-3-chloroacrylic acid (1.9 g, 62.9% yield). LCMS Calcd for [M−H]⁺, 104.98, found 105.1

### Step-2: Synthesis of (Z)-3-chloroacryloyl chloride

To a stirred solution of acetonitrile (3 mL) and DMF (3 drop) under N₂ atmosphere was added of (Z)-3-chloroacrylic acid (0.2 g, 1.87 mmol). After 10 min this solution was cooled 0-5° C. Oxalyl chloride (0.122 g, 0.968 mmol) was added and the reaction mixture was maintained at 0-5° C. for 30 min. It was allowed to warm to RT and stirring was continued for 2 h to get (Z)-3-chloroacryloyl chloride. Formation of product was confirmed by TLC and the reaction mass was used as such to the next step without any workup.

### Step-3: Synthesis of (E)-3-((3-(3-benzyl-7-((1-methyl-1H-pyrazol-4-yl)amino)-2-oxo-3,4-dihydropyrimido[4,5-d]pyrimidin-1(2H)-yl)phenyl)amino)acryloyl chloride (Compound 50)

A solution of 1-(3-Aminophenyl)-3-benzyl-7-((1-methyl-1H-pyrazol-4-yl)amino)-3,4-dihydropyrimido[4,5-d]pyrimidin-2(1H)-one (0.11 g, 0.7 mmol) in DMP (2 mL) was cooled to −15° C. and then (Z)-3-chloroacryloyl chloride was added. The reaction mixture was stirred for 1 h at −15° C. to RT. The reaction was monitored by TLC. After the completion of reaction, reaction mass was quenched with ice water and sodium bicarbonate solution. The aqueous layer was e 0.028 g, 22% yield) as a white solid. ¹H NMR (400 MHz, DMSO-d₆): δ 10.35 (s, 1H), 9.32 (s, 1H), 8.06 (s, 1H), 7.74 (s, 1H), 7.59 (s, 1H), 7.51 (s, 1H), 7.41-7.35 (m, 5H), 7.30-7.29 (m, 1H), 7.08-7.02 (m, 2H), 6.62-6.58 (m, 2H), 4.62 (s, 2H), 4.37 (s, 2H), 3.47 (s, 3H); LCMS Calcd for [M+H]⁺ 515.1, LCMS found 515.2

### Compound 51: (E)-N-(3-(7-((3-chloro-1-methyl-1H-pyrazol-4-yl)amino)-3-phenyl-2-thioxo-3,4-dihydropyrimido[4,5-d]pyrimidin-1(2H)-yl)phenyl)-4-(dimethylamino)but-2-enamide

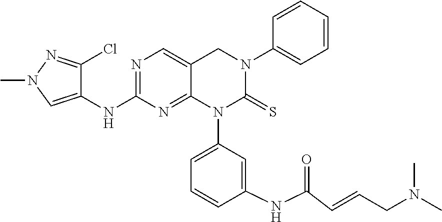

### Step-1: Synthesis of 2,4-dichloro-5-(chloromethyl)pyrimidine

Title compound was prepared in a similar manner to general procedure I. 5-(hydroxymethyl)pyrimidine-2,4-diol (15 g, 106 mmol) gave 2,4-dichloro-5-(chloromethyl)pyrimidine (11.50 g, 55% yield) as a white solid. ¹H NMR (400 MHz, CDCl₃): δ 8.66 (s, 1H), 4.65 (s, 2H).

### Step-2: Synthesis of 2,4-dichloro-5-(iodomethyl)pyrimidine

Title compound was prepared in a similar manner to general procedure J. 2,4-dichloro-5-(chloromethyl)pyrimidine (11.50 g, 58.20 mmol) on treatment with NaI (10.50 g, 69.0 mmol) in acetone (100 mL) resulted in 2,4-dichloro-5-(iodomethyl)pyrimidine (15.20 g, 91% yield). The solid was immediately taken up in toluene and stored under refrigeration. ¹H NMR (400 MHz, CDCl₃): δ 8.60 (s, 1H), 4.39 (s, 2H).

### Step-3: Synthesis of N-((2,4-dichloropyrimidin-5-yl)methyl)aniline

A solution of iodo compound (18, 7.0 g, 24.20 mmol) in toluene (50 mL) was cooled to 0° C. and aniline (2.20 g, 24.20 mmol) was added. The reaction mixture was stirred for 30 min at 0° C. Then a solution of sodium hydroxide (1.30 g, 32.50 mmol) in water (5 ml) was added and reaction mixture was stirred for 16 h at RT. The reaction was monitored by TLC. After completion of the reaction, water (25 mL) was added and extracted with ethyl acetate (2×100 mL). The organic layer was washed with brine solution, dried over anhydrous sodium sulfate and evaporated under reduced pressure to obtain the crude residue. The crude compound was purified by silica gel column chromatography to afford the title compound as a white solid (10 g, 81% yield). LCMS Calcd for [M+H]⁺ 254.11, found 254.09

### Step-4: Synthesis of tert-butyl (3-((2-chloro-5-((phenylamino)methyl)pyrimidin-4-yl)amino)phenyl)carbamate

To a stirred solution of N-((2,4-dichloropyrimidin-5-yl)methyl)aniline (4.0 g, 15.08 mmol) in IPA (30 mL), tert-butyl (3-aminophenyl)carbamate (4.90 g, 23.0 mmol) and DIPEA (8.20 mL, 47 mmol) were added. The reaction mixture was heated at 100° C. for 16 h in a sealed tube. Solvent was then evaporated and the crude thus obtained was purified by flash column chromatography to afford the title compound as off white solid (2.50 g, 37% yield). LCMS Calcd for [M+H]⁺ 425.92, found 426.35

### Step-5: Synthesis of tert-butyl (3-(7-chloro-3-phenyl-2-thioxo-3,4-dihydropyrimido[4,5-d]pyrimidin-1(2H)-yl)phenyl)carbamate

To a solution of tert-butyl (3-((2-chloro-5-((phenylamino)methyl)pyrimidin-4-yl)amino)phenyl)carbamate (1.50 g, 3.50 mmol) in THE (35 mL) was added DIPEA (2.40 mL, 14.10 mmol) and thiophosgene (0.27 g, 3.50 mmol) at 0° C. The reaction mixture was stirred at RT for 24 h with TLC monitoring. After completion of the reaction, sodium bicarbonate solution was added. The reaction mixture was partitioned between DCM (2×100 mL) and water (50 mL). The organic layer was washed with brine (10 mL), dried over anhydrous sodium sulfate and evaporated under reduced pressure to obtain crude product. The crude product was purified by silica gel column chromatography to afford the title compound as a yellow solid (1.36 g, 82% yield). LCMS Calcd for [M+H]⁺ 467.97, found 468.27

### Step-6: Synthesis of tert-butyl (3-(7-((3-chloro-1-methyl-1H-pyrazol-4-yl)amino)-3-phenyl-2-thioxo-3,4-dihydropyrimido[4,5-d]pyrimidin-1(2H)-yl)phenyl)carbamate

To a solution of tert-butyl (3-(7-chloro-3-phenyl-2-thioxo-3,4-dihydropyrimido[4,5-d]pyrimidin-1(2H)-yl)phenyl)carbamate (1.30 g, 2.78 mmol) in IPA (15 mL) was added 3-chloro-1-methyl-1H-pyrazol-4-amine (0.44 g, 3.34 mmol) and TFA (1 mL). The reaction mixture was heated for 16 h at 110° C. Reaction was monitored by TLC. After the completion of reaction, the reaction mixture was concentrated, water (10 mL) and saturated sodium bicarbonate (20 mL) solution were added to the residue and extracted with DCM (3×200 mL). The combined organic layer was washed with brine solution, dried over anhydrous sodium sulfate and evaporated under reduced pressure to obtain the title compound (1.30 g) that was used as such for the next step without further purification. LCMS Calcd for [M+H]⁺ 563.08, found 562.90

### Step-7: Synthesis of 1-(3-aminophenyl)-7-((3-chloro-1-methyl-1H-pyrazol-4-yl)amino)-3-phenyl-3,4-dihydropyrimido[4,5-d]pyrimidine-2(1H)-thione

To an ice-cold solution of tert-butyl (3-(7-((3-chloro-1-methyl-1H-pyrazol-4-yl)amino)-3-phenyl-2-thioxo-3,4-dihydropyrimido[4,5-d]pyrimidin-1(2H)-yl)phenyl)carbamate (1.30 g, 2.30 mmol) in DCM (20 mL) and MeOH (10 mL) was added 4N HCl in dioxane (5 mL). The reaction mixture was stirred for 16 h at RT. The reaction was monitored by TLC. After completion of the reaction, the solvent was evaporated followed by addition of water (10 mL) and saturated sodium bicarbonate (20 mL) solution and extraction with DCM (3×200 mL). The combined organic layer was washed with brine solution, dried over anhydrous sodium sulfate and evaporated under reduced pressure to obtain crude product. The crude product was purified by silica gel column chromatography to afford the title compound as a brown solid (0.20 g). LCMS Calcd for [M+H]⁺ 462.96, found 463.0. Purity: 68%

### Step-8: Synthesis of (E)-N-(3-(7-((3-chloro-1-methyl-1H-pyrazol-4-yl)amino)-3-phenyl-2-thioxo-3,4-dihydropyrimido[4,5-d]pyrimidin-1(2H)-yl)phenyl)-4-(dimethylamino)but-2-enamide (Compound 51)

To an ice-cold solution of 1-(3-aminophenyl)-7-((3-chloro-1-methyl-1H-pyrazol-4-yl)amino)-3-phenyl-3,4-dihydropyrimido[4,5-d]pyrimidine-2(1H)-thione (0.18 g, 0.39 mmol) and trans-N,N-dimethylaminocrotonic acid hydrochloride (0.077 g, 0.47 mmol) in dichloromethane (10 mL) was added triethyl amine (1.2 mmol) followed by drop wise addition of propylphosphonic anhydride (T₃P) (0.26 g, 0.97 mmol). The mixture was stirred at RT for 6 h. Completion of the reaction was monitored by TLC. The reaction mixture was portioned between 5% methanol in dichloromethane and saturated bicarbonate solution. The organic phase was dried over anhydrous sodium sulfate, filtered and concentrated. The crude obtained was purified by silica gel chromatography to afford the title compound as off white solid (Compound 51, 0.010 g, 5% yield). ¹H NMR (400 MHz, DMSO-d₆): δ 10.36 (bs, 1H), 8.97 (bs, 1H), 8.25 (s, 1H), 7.72 (bs, 2H), 7.48-7.42 (m, 5H), 7.36-7.32 (m, 1H), 7.03 (d, J=7.6 Hz, 1H), 6.76-6.60 (m, 2H), 6.30 (d, J=14.8 Hz, 1H), 4.95 (s, 2H), 3.50 (s, 3H), 3.12 (bs, 2H), 2.21 (s, 6H); LCMS Calcd for [M+H]⁺ 574.10, found 574.41

### Scheme 28: Preparation of (E)-N-(3-(3-benzyl-7-((1-methyl-1H-pyrazol-3-yl)amino)-2-oxo-3,4-dihydropyrimido[4,5-d]pyrimidin-1(2H)-yl)phenyl)-4-(dimethylamino)but-2-enamide (Compound 52)

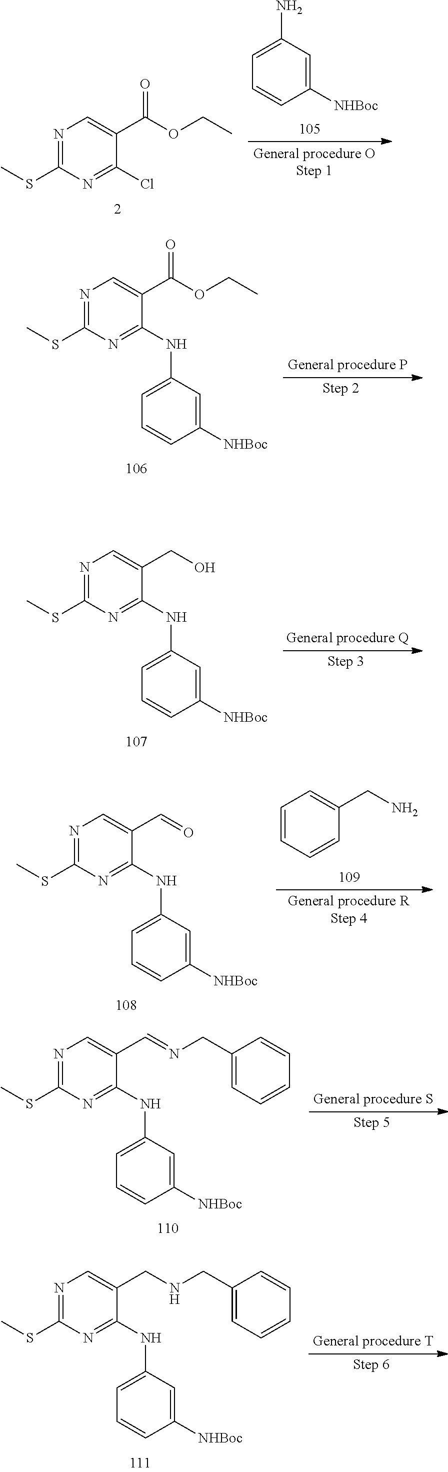

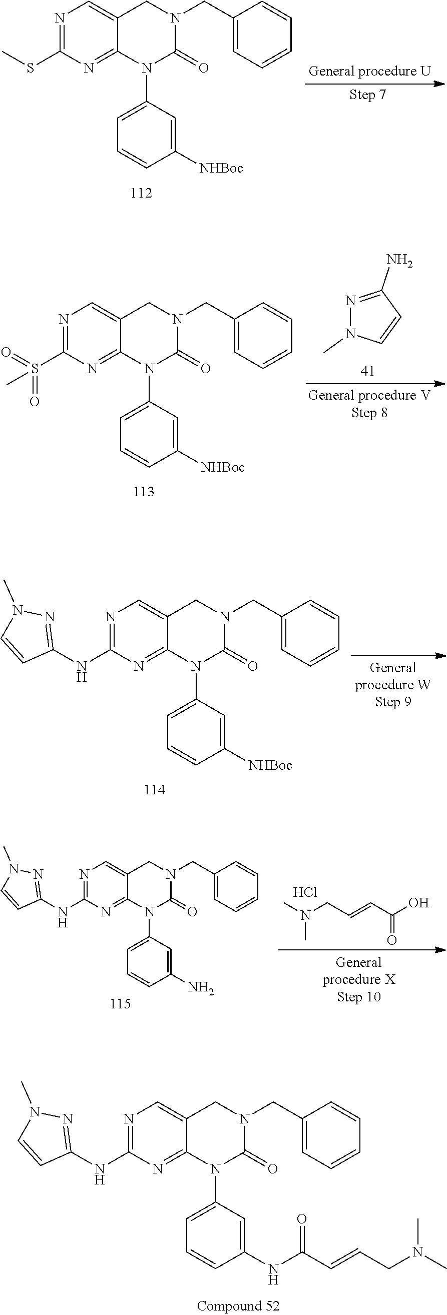

### Step 1: Preparation of ethyl 4-((3-((tert-butoxycarbonyl) amino) phenyl) amino)-2-(methylthio) pyrimidine-5-carboxylate (106)

Title compound (106) was prepared as off-white solid (142 g; Yield: 74%) in a manner substantially similar to procedure mentioned in General procedure O. ¹H-NMR (400 MHz, CDCl₃): δ 10.36 (s, 1H), 8.77 (d, 1H), 7.89 (s, 1H), 7.35 (d, J=8.0 Hz, 1H), 7.25-7.22 (m, 1H), 7.03 (d, J=8.0 Hz, 1H), 6.51 (s, 1H), 4.35 (q, J=7.2 Hz, 2H), 2.54 (s, 3H), 1.51 (s, 9H), 1.42-1.38 (m, 3H). LCMS: [M+H]⁺ 405.21, 89.28%.

### Step 2: Preparation of tert-butyl (3-((5-(hydroxymethyl)-2-(methylthio)pyrimidin-4-yl)amino)phenyl)carbamate (107)

Title compound was prepared in a manner substantially similar to procedure mentioned in General procedure P. The crude was triturated with dichloromethane afforded 107 as off white solid (40.0 g; Yield: 31%). ¹H-NMR (400 MHz, CDCl₃): δ 8.09 (s, 1H), 7.86 (m, 2H), 7.36 (d, J=8.0 Hz, 1H), 7.25-7.15 (m, 1H), 6.95 (d, J=8.0 Hz, 1H), 6.55 (s, 1H), 4.59 (s, 2H), 2.50 (s, 3H), 1.51 (s, 9H). LCMS: [M+H]⁺ 363.05, 91.24%.

### Step 3: Preparation of tert-butyl (3-((5-formyl-2-(methylthio)pyrimidin-4-yl)amino)phenyl)carbamate (108)

Title compound (108) was prepared as a pale yellow solid (31.0 g; Yield: 78%) in a manner substantially similar to procedure mentioned in General procedure Q. ¹H-NMR (400 MHz, CDCl₃): δ 10.59 (s, 1H), 9.75 (s, 1H), 8.42 (s, 1H), 7.97 (s, 1H), 7.35 (d, J=8.0 Hz, 1H), 7.04 (d, J=8.0 Hz, 1H), 6.59 (s, 1H), 3.48 (s, 1H), 2.58 (s, 3H), 1.52 (s, 9H). LCMS: [M+H]⁺ 361.30, 97.51%.

### Step 4: Preparation of tert-butyl (E)-(3-((5-((benzylimino)methyl)-2(methylthio)pyrimidin-4-yl)amino)phenyl)carbamate (110)

Title compound (110) was prepared as a yellow solid (28 g; Yield: 72%) in a manner substantially similar to procedure mentioned in General procedure R. ¹H-NMR (400 MHz, CDCl₃): δ 12.15 (s, 1H), 8.31 (s, 1H), 8.16 (s, 1H), 7.91 (s, 1H), 7.41 (m, 4H), 7.35-7.33 (m, 1H), 7.32-7.29 (m, 1H), 7.26-7.22 (m, 1H), 7.03 (d, J=8.0 Hz, 1H), 6.46 (s, 1H), 4.84 (s, 2H), 2.59 (s, 3H), 1.52 (s, 9H). LCMS: [M+H]⁺ 450.38; 99.66%.

### Step 5: Preparation of tert-butyl (3-((5-((benzylamino)methyl)-2-(methylthio)pyrimidin-4-yl)amino)phenyl)carbamate (111)

Title compound (111) was prepared as a pale yellow solid (40 g; Yield: 80%) in a manner substantially similar to procedure mentioned in General procedure S. LCMS: [M+H]⁺ 452.44; 83.57%

### Step 6: Preparation of tert-butyl (3-(3-benzyl-7-(methylthio)-2-oxo-3,4-dihydropyrimido[4,5-d]pyrimidin-1(2H)-yl)phenyl)carbamate (112)

Title compound was prepared in a manner substantially similar to procedure mentioned in General procedure T. The crude was triturated with diethyl ether afforded 112 as off white solid (12 g; Yield: 28%). ¹H-NMR (400 MHz, CDCl₃): δ 8.03 (s, 1H), 7.50 (s, 1H), 7.37 (m, 6H), 7.26 (m, 1H), 6.96 (m, 1H), 6.59 (s, 1H), 4.69 (s, 2H), 4.34 (s, 2H), 2.16 (s, 3H), 1.50 (s, 9H). LCMS: [M+H]⁺ 478.16; 95.62%.

### Step 7: Preparation of tert-butyl (3-(3-benzyl-7-(methylsulfonyl)-2-oxo-3,4-dihydropyrimido [4,5-d]pyrimidin-1(2H)-yl)phenyl)carbamate (113)

Title compound was prepared in a manner substantially similar to procedure mentioned in General procedure U. The crude was triturated with diethyl ether afforded 113 as an off white solid (8.0 g; Yield: 76%). ¹H-NMR (400 MHz, CDCl₃): δ 8.39 (s, 1H), 7.63 (s, 1H), 7.40 (m, 6H), 7.17 (d, J=8.0 Hz, 1H), 6.95 (d, J=8.0 Hz, 1H), 6.61 (s, 1H), 4.71 (s, 2H), 4.48 (s, 2H), 2.97 (s, 3H), 1.49 (s, 9H). LCMS: [M+H]⁺ 510.31, 93.69%.

### Step 8: Preparation of tert-butyl (3-(3-benzyl-7-((1-methyl-1H-pyrazol-3-yl)amino)-2-oxo-3,4-dihydropyrimido[4,5-d]pyrimidin-1(2H)-yl)phenyl)carbamate (114)

Title compound was prepared in a manner substantially similar to General procedure V, tert-butyl (3-(3-benzyl-7-(methylsulfonyl)-2-oxo-3,4-dihydropyrimido[4,5-d]pyrimidin-1(2H)-yl)phenyl)carbamate (113) and 1-methyl-1H-pyrazol-3-amine (41) gave (tert-butyl (3-(3-benzyl-7-((1-methyl-1H-pyrazol-3-yl)amino)-2-oxo-3,4-dihydropyrimido[4,5-d]pyrimidin-1(2H)-yl)phenyl)carbamate (114) as a brown solid (Yield: 77%), which was used directly for the next step without any further purification. MS: [M+H]⁺ 527.46.

### Step 9: Preparation of 1-(3-aminophenyl)-3-benzyl-7-((1-methyl-1H-pyrazol-3-yl)amino)-3,4-dihydropyrimido[4,5-d]pyrimidin-2(1H)-one (115)

Title compound was prepared in a manner substantially similar to General procedure W, tert-butyl (3-(3-benzyl-7-((1-methyl-1H-pyrazol-3-yl)amino)-2-oxo-3,4-dihydropyrimido[4,5-d]pyrimidin-1(2H)-yl)phenyl)carbamate (114) gave 1-(3-aminophenyl)-3-benzyl-7-((1-methyl-1H-pyrazol-3-yl)amino)-3,4-dihydropyrimido[4,5-d]pyrimidin-2(1H)-one (115) as a brown solid (Yield: 93%), which was used directly for the next step. MS: [M+H]⁺ 427.44.

### Step 10: Preparation of (E)-N-(3-(3-benzyl-7-((1-methyl-1H-pyrazol-3-yl)amino)-2-oxo-3,4-dihydropyrimido[4,5-d]pyrimidin-1(2H)-yl)phenyl)-4-(dimethylamino)but-2-enamide (Compound 52)

Title compound was prepared in a manner substantially similar General procedure X, 1-(3-aminophenyl)-3-benzyl-7-((1-methyl-1H-pyrazol-3-yl)amino)-3,4-dihydropyrimido[4,5-d]pyrimidin-2(1H)-one (115) and trans-N,N-dimethylaminocrotonic acid hydrochloride gave (E)-N-(3-(3-benzyl-7-((1-methyl-1H-pyrazol-3-yl)amino)-2-oxo-3,4-dihydropyrimido[4,5-d]pyrimidin-1(2H)-yl)phenyl)-4-(dimethylamino)but-2-enamide Compound 52, as a white solid (48 mg; Yield: 13%), after prep-HPLC purification. ¹H-NMR (400 MHz, CDCl₃): δ 10.17 (s, 1H), 9.51 (s, 1H), 8.08 (s, 1H), 7.72 (d, J=8.4 Hz, 1H), 7.60 (s, 1H), 7.43-7.35 (m, 5H), 7.33-7.29 (m, 1H), 7.10 (s, 1H), 7.01 (d, J=8.8 Hz, 1H), 6.75-6.69 (m, 1H), 6.27 (d, J=15.3 Hz, 1H), 5.51 (s, 1H), 4.62 (s, 2H), 4.39 (s, 2H), 3.59 (s, 3H), 3.06 (d, J=4.8 Hz, 2H), 2.17 (s, 6H). MS: [M+H]⁺ 538.32.

The following compounds were prepared using the methods described above

| | | Synthe- | | |
|---|---|---|---|---|
| Cmpd | | sis | LCMS | |
| No. | Structure | Method | [M + H]⁺ | ¹H-NMR (400 MHz, DMSO-d₆) |
| 53 | 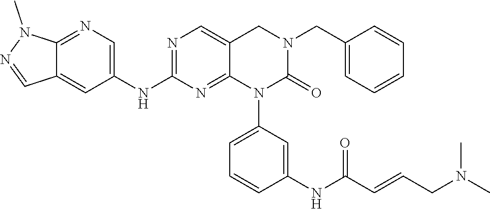 [SMILES: CN(C)C/C=C/C(=O)Nc1cccc(N2C(=O)N(Cc3ccccc3)Cc3cnc(Nc4cnc5c(cnn5C)c4)nc32)c1] | X | 589.30 | δ 10.28 (s, 1H), 9.73 (s, 1H), 8.44 (d, J = 1.2 Hz, 1H), 8.19 (s, 1H), 7.97 (s, 1H), 7.83 (d, J = 8.0 Hz, 1H), 7.78 (s, 1H), 7.70 (s, 1H), 7.50 -7.48 (t, J = 8.0 Hz, 1H), 7.41-7.37 (m, 4H), 7.33-7.30 (m, 1H), 7.06 (d, J = 7.6 Hz, 1H), 6.73-6.66 (m, 1H), 6.26 (d, J = 15.44 Hz, 1H), 4.65 (s, 2H), 4.44 (s, 2H), 3.94 (s, 3H), 3.03 (d, J = 5.6 Hz, 2H), 2.14 (s, 6H). |
| 54 | 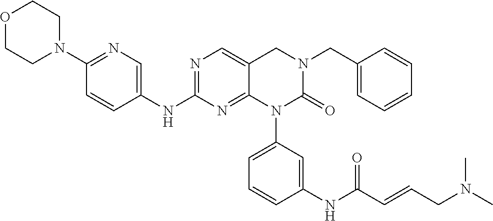 [SMILES: CN(C)C/C=C/C(=O)Nc1cccc(N2C(=O)N(Cc3ccccc3)Cc3cnc(Nc4ccc(N5CCOCC5)nc4)nc32)c1] | X | 620.33 | δ 10.2 (s, 1H), 9.20 (s, 1H), 8.10 (s, 1H), 8.04 (s, 1H), 7.78 (d, 1H), 7.58 (s, 1H), 7.52 (m, 1H), 7.45-7.37 (m, 5H), 7.32 (d, 1H), 7.01 (d, J = 7.6 Hz, 1H), 6.75-6.69 (m, 1H), 6.36 (s, 1H), 6.31 (d, J = 15.4 Hz, 1H), 4.4 (s, 2H), 4.39 (s, 2H), 3.66 (d, J = 4.4 Hz, 4H), 3.23 (s, 4H), 3.05 (d, J = 5.6 Hz, 2H), 2.16 (s, 6H). |
| 55 | 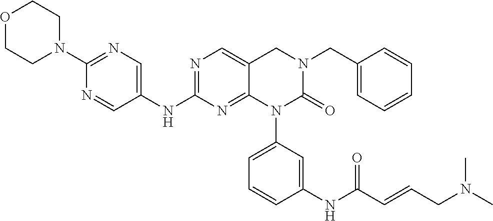 [SMILES: CN(C)C/C=C/C(=O)Nc1cccc(N2C(=O)N(Cc3ccccc3)Cc3cnc(Nc4cnc(N5CCOCC5)nc4)nc32)c1] | X | 621.34 | δ 10.15 (s, 1H), 9.09 (s, 1H), 8.30 (s, 2H), 8.10 (s, 1H), 7.68-7.64 (m, 2H), 7.40-7.29 (m, 6H), 6.99 (d, J = 8.4 Hz, 1H), 6.74-6.67 (m, 1H), 6.28 (d, J = 15.4 Hz, 1H), 4.62 (s, 2H), 4.39 (s, 2H), 3.62-3.60 (m, 4H), 3.54 (d, J = 4.4 Hz, 4H), 3.05 (d, J = 6.0 Hz, 2H), 2.16 (s, 6H). |
| 56 | 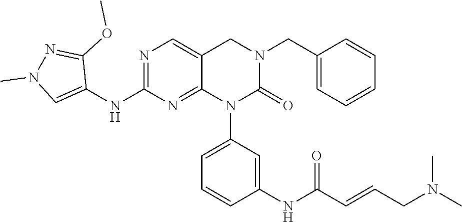 | X | 568.32 | δ 10.24 (s, 1H), 8.41 (bs, 1H), 8.06 (s, 1H), 7.67-7.78 (m, 1H), 7.63 (s, 1H), 7.46-7.50 (m, 1H), 7.28-7.41 (m, 5H), 7.03 (d, J = 7.8 Hz, 1H), 6.70-6.77 (m, 1H), 6.45 (bs, 1H), 6.27 (d, J = 15.4 Hz, 1H), 4.63 (s, 2H), 4.38 (s, 2H), 3.71 (s, 3H), 3.32-3.65 (bs, 3H), 3.05 (d, J = 4.8 Hz, 2H), 2.17 (s, 6H). |
| 57 | 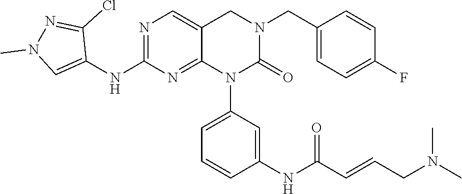 | X | 590.35 | δ 10.24 (s, 1H), 8.76 (bs, 1H), 8.12 (s, 1H), 7.75 (m, 1H), 7.65 (s, 1H), 7.50- 7.46 (m, 1H), 7.43-7.40 (m, 2H), 7.23- 7.18 (m, 2H), 7.04 (d, J = 7.8 Hz, 1H), 6.76-6.69 (m, 2H), 6.28 (d, J = 15.4 Hz, 1H), 4.61 (s, 2H), 4.40 (s, 2H), 3.48 (s, 3H), 3.05 (d, J = 5.8 Hz, 2H), 2.16 (s, 6H). |
| 58 | 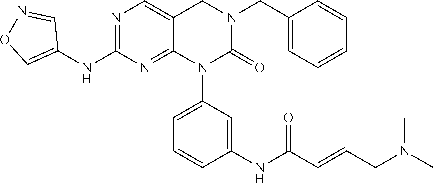 [SMILES: CN(C)C/C=C/C(=O)Nc1cccc(N2C(=O)N(Cc3ccccc3)Cc3cnc(Nc4cnoc4)nc32)c1] | X | 525.28 | δ 10.25 (s, 1H), 9.62 (s, 1H), 8.25 (s, 1H), 8.17 (s, 1H), 7.77 (s, 1H), 7.69 (m, 1H), 7.50-7.46 (m, 2H), 7.41-7.37 (m, 4H), 7.33-7.29 (m, 1H), 7.06 (d, J = 7.2 Hz, 1H), 6.75-6.70 (m, 1H), 6.28 (d, J = 15.4 Hz, 1H), 4.64 (s, 2H), 4.42 (s, 2H), 3.06 (d, J = 5.2 Hz, 2H), 2.16 (s, 6H). |
| 59 | 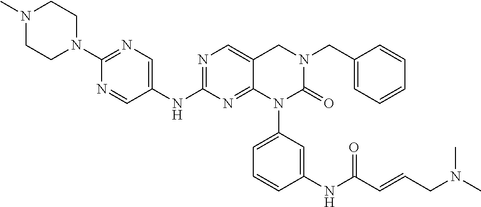 | X | 634.28 | δ 10.15 (s, 1H), 9.05 (s, 1H), 8.27 (s, 2H), 8.09 (s, 1H), 7.70 (d, J = 8.3 Hz, 1H), 7.63 (s, 1H), 7.40-7.29 (m, 6H), 6.98 (d, 1H), 6.74-6.68 (m, 1H), 6.28 (d, J = 15.3 Hz, 1H), 4.62 (s, 2H), 4.38 (s, 2H), 3.57 (s, 4H), 3.05 (d, J = 5.6 Hz, 2H), 2.30-2.28 (m, 4H), 2.18 (m, 9H). |
| 60 | 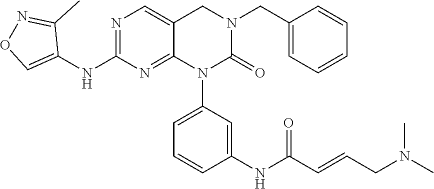 [SMILES: Cc1nocc1Nc1ncc2c(n1)N(c1cccc(NC(=O)/C=C/CN(C)C)c1)C(=O)N(Cc1ccccc1)C2] | X | 539.33 | δ 10.24 (s, 1H), 9.19 (s, 1H), 8.19 (s, 1H), 7.76 (s, 1H), 7.67 (m, 1H), 7.49- 7.45 (m, 1H), 7.41-7.29 (m, 6H), 7.05 (d, J = 8.0 Hz, 1H), 6.75-6.68 (m, 1H), 6.28 (d, J = 15.4 Hz, 1H), 4.64 (s, 2H), 4.43 (s, 2H), 3.05 (d, J = 5.2 Hz, 2H), 2.20 (s, 3H), 2.16 (s, 6H). |
| 61 | 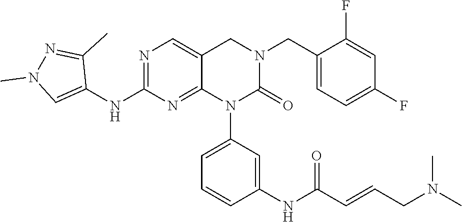 | X | 608.15 | δ 10.24 (s, 1H), 8.78 (s, 1H), 8.14 (s, 1H), 7.73 (d, J = 8.0 Hz, 1H), 7.64 (s, 1H), 7.76 (m, 2H), 7.26 (m, 1H), 7.09 (m, 1H), 7.01 (d, J = 8.8 Hz, 1H), 6.69 (m, 2H), 6.24 (d, J = 15.2 Hz, 1H), 4.64 (s, 2H), 4.45 (s, 2H), 3.48 (s, 3H), 3.03 (d, J = 5.6 Hz, 2H), 2.16 (s, 6H). |
| 62 | 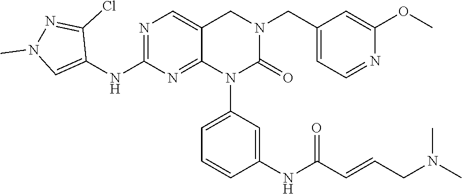 | X | 603.21 | δ 10.25 (s, 1H), 8.80 (s, 1H), 8.13 (m, 2H), 7.74 (d, J = 8.4 Hz, 1H), 7.65 (s, 1H), 7.46 (t, J = 8.0 Hz, 1H), 7.04 (d, J = 7.6 Hz, 1H), 6.95 (d, J = 4.4 Hz, 1H), 6.78 (s, 1H), 6.69 (m, 2H), 6.24 (d, J = 15.6 Hz, 1H), 4.61 (s, 2H), 4.45 (s, 2H), 3.84 (s, 3H), 3.48 (s, 3H), 3.04 (d, J = 5.5 Hz, 2H), 2.16 (s, 6H). |
| 63 | 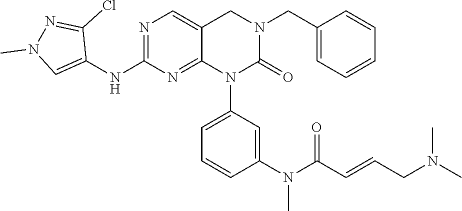 | X | 586.17 | δ 8.78 (bs, 1H), 8.12 (s, 1H), 7.66 (m, 1H), 7.51 (m, 1H), 7.37 (m, 5H), 7.29 (m, 1H), 7.24 (s, 1H), 6.70 (m, 1H), 6.53 (m, 1H), 5.87 (m, 1H), 4.63 (s, 2H), 4.39 (s, 2H), 3.49 (s, 3H), 3.26 (s, 3H), 2.50 (m, 2H), 1.90 (s, 6H). |
| 64 | 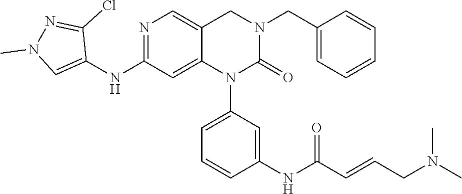 | X | 571.22 | δ 10.27 (s, 1H), 8.19 (s, 1H), 7.99 (s, 1H), 7.81 (s, 1H), 7.72 (d, J = 8.8 Hz, 1H), 7.65 (s, 1H), 7.46 (t, J = 8.4 Hz, 1H), 7.28 (m, 5H), 6.99 (d, J = 8.4 Hz, 1H), 6.71 (m, 1H), 6.26 (d, J = 15.44 Hz, 1H), 5.70 (s, 1H), 4.60 (s, 2H), 4.39 (s, 2H), 3.72 (s, 3H), 3.05 (d, J = 5.32 Hz, 2H), 2.17 (s, 6H). |
| 65 | 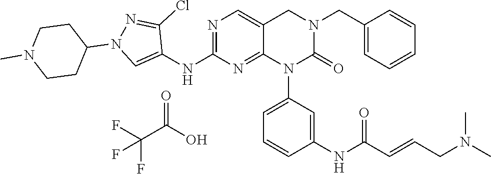 | X | 655.39 | δ 10.69 (bs, 1H), 10.14 (bs, 1H), 9.76 (bs, 1H), 8.86 (bs, 1H), 8.14 (s, 1H), 7.98 (bs, 1H), 7.55 (bs, 1H), 7.41-7.35 (m, 2H), 7.33-7.30 (m, 1H), 7.26-7.24 (m, 2H), 7.01-6.98 (m, 2H), 6.79-6.72 (m, 1H), 6.50 (d, J = 15.2 Hz, 1H), 4.63 (s, 2H), 4.42 (s, 2H), 3.94 (d, J = 5.6 Hz, 2H), 3.49 (bs, 1H), 3.29-3.25 (m, 4H), 2.84 (s, 3H), 2.79 (s, 6H), 2.09- 2.00 (m, 4H). |
| 66 | 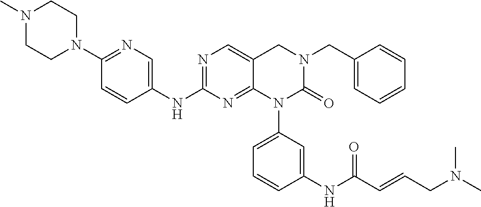 | X | 633.49 | δ 10.20 (bs, 1H), 8.09 (s, 1H), 8.09 (bs, 1H), 7.77 (d, J = 8.0 Hz, 1H), 7.58 (s, 1H), 7.29 (m, 8H), 6.99 (d, J = 7.6 Hz, 1H), 6.71 (m, 2H), 6.24 (d, J = 15.6 Hz, 1H), 4.61 (s, 2H), 4.39 (s, 2H), 3.28 (m, 4H), 3.04 (m, 2H), 2.34 (m, 4H), 2.19 (s, 3H), 2.16 (s, 6H). |
| 67 | 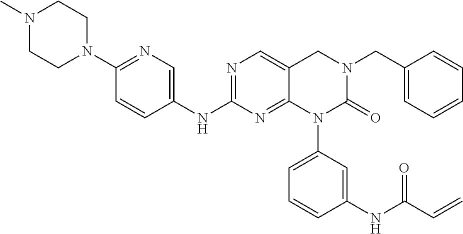 [SMILES: C=CC(=O)Nc1cccc(N2C(=O)N(Cc3ccccc3)Cc3cnc(Nc4ccc(N5CCN(C)CC5)nc4)nc32)c1] | Y | 576.39 | δ 10.21 (bs, 1H), 9.15 (bs, 1H), 8.09 (s, 1H), 8.01 (bs, 1H), 7.79 (d, J = 8.0 Hz, 1H), 7.58 (s, 1H), 7.30-7.47 (m, 7H), 7.01 (d, J = 7.6 Hz, 1H), 6.35-6.46 (m, 2H), 6.22 (d, J = 16.8 Hz, 1H), 5.74 (d, J = 10.4 Hz, 1H), 4.63 (s, 2H), 4.39 (s, 2H), 3.27 (m, 4H), 2.34-2.36 (m, 4H), 2.19 (s, 3H). |
| 68 | 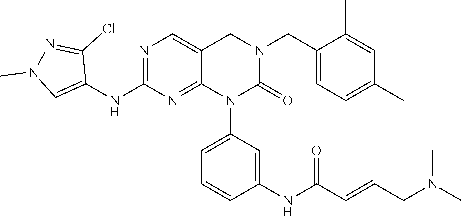 | X | 600.44 | δ 10.25 (bs, 1H), 8.75 (bs, 1H), 8.10 (s, 1H), 7.75 (d, J = 8.0 Hz, 1H), 7.63 (s, 1H), 7.47-7.51 (m, 1H), 7.15-7.17 (m, 1H), 7.01-7.03 (m, 3H), 6.70-6.76 (m, 2H), 6.24 (d, J = 15.4 Hz, 1H), 4.60 (s, 2H), 4.32 (s, 2H), 3.48 (s, 3H), 3.04 (d, J = 5.6 Hz, 2H), 2.25 (s, 6H), 2.16 (s, 6H). |
| 69 | 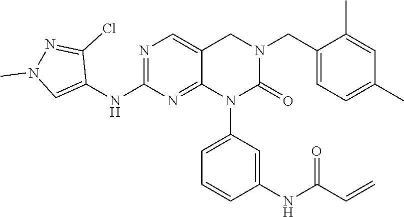 | Y | 543.33 | δ 10.34 (bs, 1H), 8.75 (bs, 1H), 8.10 (s, 1H), 7.78 (d, J = 8.0 Hz, 1H), 7.63 (s, 1H), 7.48-7.52 (m, 1H), 7.15-7.17 (m, 1H), 7.01-7.03 (m, 3H), 6.70 (s, 1H), 6.40-6.46 (m, 1H), 6.23-6.28 (m, 1H), 5.75 (d, J = 10.0 Hz, 1H), 4.60 (s, 2H), 4.32 (s, 2H), 3.48 (s, 3H), 2.25 (s, 6H) |
| 70 | 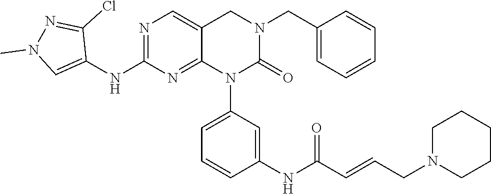 [SMILES: Cn1cc(Nc2ncc3c(n2)N(c2cccc(NC(=O)/C=C/CN4CCCCC4)c2)C(=O)N(Cc2ccccc2)C3)c(Cl)n1] | X | 612.38 | δ 10.24 (bs, 1H), 8.76 (bs, 1H), 8.12 (s, 1H), 7.74 (d, J = 8.0 Hz, 1H), 7.64 (s, 1H), 7.40-7.49 (m, 1H), 7.29-7.32 (m, 5H), 7.03 (d, J = 8.0 Hz, 1H), 6.70-6.76 (m, 2H), 6.22 (d, J = 15.2 Hz, 1H), 4.63 (s, 2H), 4.40 (s, 2H), 3.48 (s, 3H), 3.06 (d, J = 5.6 Hz, 2H), 2.33 (s, 4H), 1.49- 1.50 (m, 4H), 1.37 (s, 2H). |
| 71 | 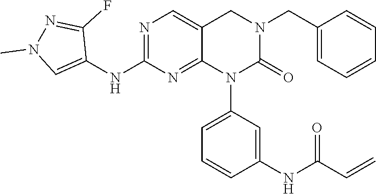 [SMILES: C=CC(=O)Nc1cccc(N2C(=O)N(Cc3ccccc3)Cc3cnc(Nc4cn(C)nc4F)nc32)c1] | Y | 499.36 | δ 10.33 (bs, 1H), 9.24 (bs, 1H), 8.10 (s, 1H), 7.78 (d, J = 6.4 Hz, 1H), 7.64 (s, 1H), 7.49 (t, J = 7.2 Hz, 1H), 7.29-7.33 (m, 5H), 7.05 (d, J = 7.6 Hz, 1H), 6.40- 6.52 (m, 2H), 6.23-6.28 (m, 1H), 5.75 (d, J = 11.6 Hz, 1H), 4.63 (s, 2H), 4.40 (s, 2H), 3.38 (s, 3H). |
| 72 | 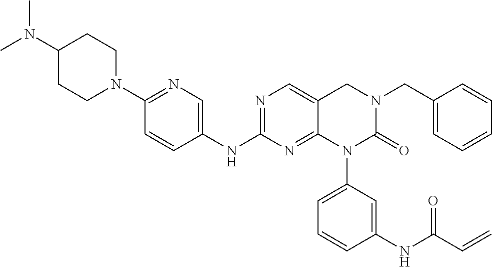 [SMILES: C=CC(=O)Nc1cccc(N2C(=O)N(Cc3ccccc3)Cc3cnc(Nc4ccc(N5CCC(N(C)C)CC5)nc4)nc32)c1] | Y | 604.47 | δ 10.29 (bs, 1H), 9.15 (bs, 1H), 8.09 (s, 1H), 7.99 (s, 1H), 7.80 (d, J = 8.0 Hz, 1H), 7.58 (s, 1H), 7.31-7.44 (m, 7H), 7.01 (d, J = 8.0 Hz, 1H), 6.35-6.46 (m, 2H), 6.23-6.27 (m, 1H), 5.74 (d, J = 10.4 Hz, 1H), 4.63 (s, 2H), 4.39 (s, 2H), 4.04-4.07 (m, 2H), 2.60-2.63 (m, 2H), 2.15-2.21 (m, 7H), 1.73-1.76 (m, 2H), 1.23-1.32 (m, 2H). |
| 73 | 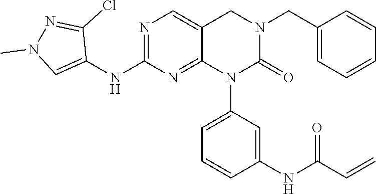 [SMILES: C=CC(=O)Nc1cccc(N2C(=O)N(Cc3ccccc3)Cc3cnc(Nc4cn(C)nc4Cl)nc32)c1] | Y | 515.37 | δ 10.33 (bs, 1H), 8.74 (bs, 1H), 8.12 (s, 1H), 7.77 (d, J = 7.6 Hz, 1H), 7.64 (s, 1H), 7.49-7.52 (m, 1H), 7.31-7.37 (m, 5H), 7.05 (d, J = 7.6 Hz, 1H), 6.70 (s, 1H), 6.40-6.46 (m, 1H), 6.23-6.28 (m, 1H), 5.75 (d, J = 10.0 Hz, 1H), 4.63 (s, 2H), 4.40 (s, 2H), 3.49 (s, 3H). |
| 74 | 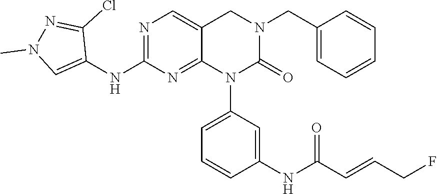 [SMILES: Cn1cc(Nc2ncc3c(n2)N(c2cccc(NC(=O)/C=C/CF)c2)C(=O)N(Cc2ccccc2)C3)c(Cl)n1] | X | 547.30 | δ 10.37 (bs, 1H), 8.76 (bs, 1H), 8.12 (s, 1H), 7.75 (d, J = 8.0 Hz, 1H), 7.65 (s, 1H), 7.46 (t, J = 8.0 Hz, 1H), 7.37-7.39 (m, 4H), 7.31-7.32 (m, 1H), 7.05 (d, J = 8.0 Hz, 1H), 6.69-6.89 (m, 2H), 6.34 (d, J = 15.6 Hz, 1H), 5.24 (s, 1H), 5.12 (s, 1H), 4.63 (s, 2H), 4.41 (s, 2H), 3.49 (s, 3H). |
| 75 | 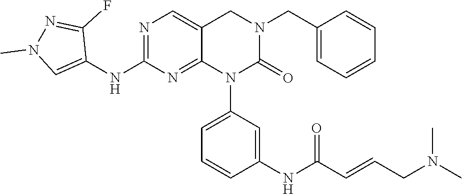 | X | 556.42 | δ 10.33 (bs, 1H), 9.24 (bs, 1H), 8.10 (s, 1H), 7.76 (d, J = 7.2 Hz, 1H), 7.64 (s, 1H), 7.48 (t, J = 7.6 Hz, 1H), 7.33-7.40 (m, 4H), 7.31-7.32 (m, 1H), 7.05 (d, J = 7.2 Hz, 1H), 6.70-6.75 (m, 1H), 6.51- 6.54 (m, 1H), 6.31 (d, J = 15.4 Hz, 1H), 4.63 (s, 2H), 4.40 (s, 2H), 3.31-3.38 (m, 5H), 2.40 (s, 6H). |
| 76 | 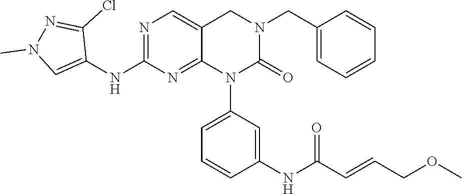 | X | 559.31 | δ 10.27 (bs, 1H), 8.72 (bs, 1H), 8.12 (s, 1H), 7.74 (d, J = 7.6 Hz, 1H), 7.65 (s, 1H), 7.47 (t, J = 8.0 Hz, 1H), 7.29-7.38 (m, 5H), 7.03 (d, J = 7.6 Hz, 1H), 6.71- 6.82 (m, 2H), 6.30 (d, J = 15.6 Hz, 1H), 4.63 (s, 2H), 4.40 (s, 2H), 4.10 (s, 2H), 3.49 (s, 3H), 3.29 (s, 3H). |
| 77 | 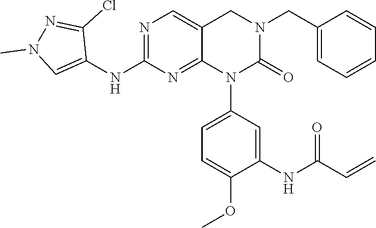 [SMILES: C=CC(=O)Nc1cc(N2C(=O)N(Cc3ccccc3)Cc3cnc(Nc4cn(C)nc4Cl)nc32)ccc1OC] | X | 546.0 | δ 9.54 (s, 1H), 8.64 (s, 1H), 8.09-8.00 (m, 2H), 7.35-7.30 (m, 5H), 7.17 (s, 1 H), 7.05 (s, 1H), 6.72 (s, 2H), 6.19 (d, J = 16.8 Hz, 1H), 5.70 (d, J = 8.4 Hz, 1H), 4.61 (s, 2H), 4.37 (s, 2H), 3.92 (s, 3H), 3.44 (s, 3H). |
| 78 | 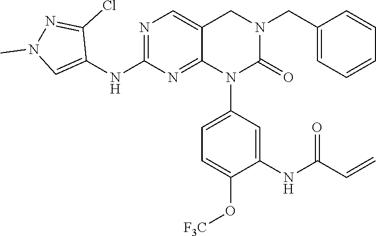 [SMILES: C=CC(=O)Nc1cc(N2C(=O)N(Cc3ccccc3)Cc3cnc(Nc4cn(C)nc4Cl)nc32)ccc1OC(F)(F)F] | X | 599.0 | δ 10.06 (s, 1H), 8.68 (s, 1H), 8.1 (s, 1H), 7.96 (s, 1H), 7.55 (d, J = 18.4 Hz, 1H), 7.35-7.16 (m, 6H), 6.98 (s, 1H), 6.67-6.60 (m, 1H), 6.25 (d, J = 16.8 Hz, 1H), 5.78 (d, J = 10.4 Hz, 1H), 4.61 (s, 2H), 4.37 (s, 2H), 3.25 (s, 3H). |
| 79 | 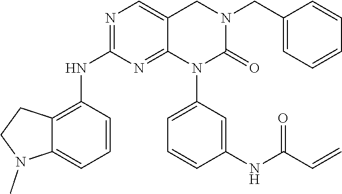 [SMILES: C=CC(=O)Nc1cccc(N2C(=O)N(Cc3ccccc3)Cc3cnc(Nc4cccc5c4CCN5C)nc32)c1] | Y | 532.25 | δ 10.26 (bs, 1H), 8.41 (bs, 1H), 8.10 (s, 1H), 7.68 (d, J = 8.4 Hz, 1H), 7.64 (s, 1H), 7.31-7.41 (m, 6H), 6.99 (d, J = 8.0 Hz, 1H), 6.66 (d, J = 8.0 Hz, 1H), 6.56 (t, J = 7.6 Hz, 1H), 6.40-6.46 (m, 1H), 6.22-6.27 (m, 1H), 6.05 (d, J = 8.0 Hz, 1H), 5.74 (d, J = 10.0 Hz, 1H), 4.63 (s, 2H), 4.39 (s, 2H), 3.08 (t, J = 8.0 Hz, 2H), 2.71 (t, J = 8.0 Hz, 2H), 2.59 (s, 3H). |
| 80 | 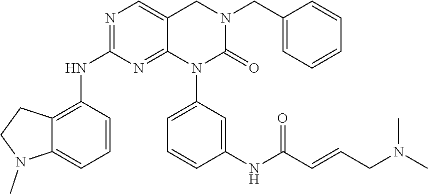 [SMILES: CN(C)C/C=C/C(=O)Nc1cccc(N2C(=O)N(Cc3ccccc3)Cc3cnc(Nc4cccc5c4CCN5C)nc32)c1] | X | 589.33 | δ 10.16 (bs, 1H), 8.40 (bs, 1H), 8.10 (s, 1H), 7.64-6.67 (m, 2H), 7.31-7.40 (m, 6H), 6.97 (d, J = 8.0 Hz, 1H), 6.66-6.75 (m, 2H), 6.54 (t, J = 8.0 Hz, 1H), 6.24 (d, J = 15.2 Hz, 1H), 6.05 (d, J = 7.6 Hz, 1H), 4.63 (s, 2H), 4.39 (s, 2H), 3.04-3.13 (m, 4H), 2.71-2.76 (m, 2H), 2.59 (s, 3H), 2.16 (s, 6H). |
| 81 | 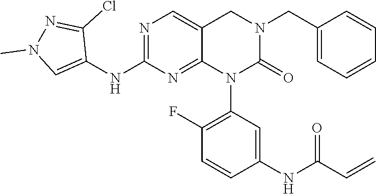 [SMILES: C=CC(=O)Nc1ccc(F)c(N2C(=O)N(Cc3ccccc3)Cc3cnc(Nc4cn(C)nc4Cl)nc32)c1] | Y | 533.17 | δ 10.37 (bs, 1H), 8.85 (bs, 1H), 8.15 (s, 1H), 7.78 (d, J = 6.4 Hz, 2H), 7.29-7.43 (m, 6H), 6.76 (bs, 1H), 6.38-6.45 (m, 1H), 6.24 (dd, J = 2.0 & 16.8 Hz, 1H), 5.76 (d, J = 10.4 Hz, 1H), 4.64 (s, 2H), 4.38 (s, 2H), 3.52 (s, 3H). |
| 82 | 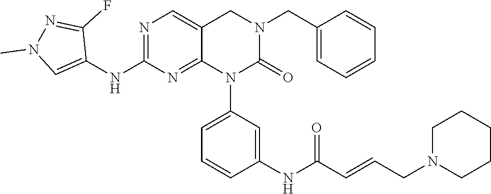 [SMILES: Cn1cc(Nc2ncc3c(n2)N(c2cccc(NC(=O)/C=C/CN4CCCCC4)c2)C(=O)N(Cc2ccccc2)C3)c(F)n1] | X | 596.25 | δ 10.23 (bs, 1H), 9.26 (bs, 1H), 8.10 (s, 1H), 7.75 (d, J = 7.2 Hz, 1H), 7.64 (s, 1H), 7.47-7.51 (m, 1H), 7.29-7.40 (m, 5H), 7.03 (d, J = 7.2 Hz, 1H), 6.70-6.76 (m, 1H), 6.50 (bs, 1H), 6.22 (d, J = 15.4 Hz, 1H), 4.63 (s, 2H), 4.39 (s, 2H), 3.38 (s, 3H), 3.06 (d, J = 5.2 Hz, 2H), 2.33 (s, 4H), 1.48-1.53 (m, 4H), 1.38- 1.39 (m, 2H). |
| 83 | 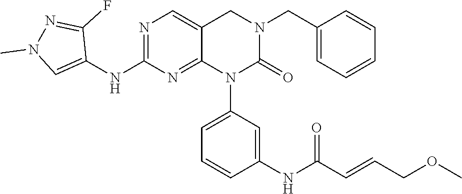 | X | 543.21 | δ 10.27 (bs, 1H), 9.24 (bs, 1H), 8.10 (s, 1H), 7.74 (d, J = 8.4 Hz, 1H), 7.65 (s, 1H), 7.47-7.50 (m, 1H), 7.29-7.40 (m, 5H), 7.03 (d, J = 7.6 Hz, 1H), 6.77-6.82 (m, 1H), 6.52 (bs, 1H), 6.30 (d, J = 15.2 Hz, 1H), 4.63 (s, 2H), 4.39 (s, 2H), 4.09-4.11 (m, 2H), 3.38 (s, 3H), 3.33 (s, 3H). |
| 84 | 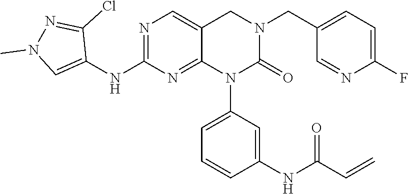 [SMILES: C=CC(=O)Nc1cccc(N2C(=O)N(Cc3ccc(F)nc3)Cc3cnc(Nc4cn(C)nc4Cl)nc32)c1] | Y | 534.13 | δ 10.33 (bs, 1H), 8.77 (bs, 1H), 8.27 (s, 1H), 8.10 (s, 1H), 7.97-8.02 (m, 1H), 7.76 (d, J = 8.0 Hz, 1H), 7.64 (s, 1H), 7.48-7.52 (m, 1H), 7.19-7.21 (m, 1H), 7.05 (d, J = 7.6 Hz, 1H), 6.68 (bs, 1H), 6.39-6.46 (m, 1H), 6.23 (dd, J = 2.0 & 17.2 Hz, 1H), 5.75 (d, J = 10.0 Hz, 1H), 4.65 (s, 2H), 4.46 (s, 2H), 3.48 (s, 3H). |
| 85 | 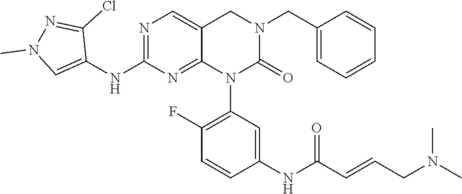 | X | 590.23 | δ 10.29 (bs, 1H), 8.85 (bs, 1H), 8.15 (s, 1H), 7.74-7.79 (m, 2H), 7.29-7.42 (m, 6H), 6.70-6.77 (m, 2H), 6.23 (d, J = 15.2 Hz, 1H), 4.64 (s, 2H), 4.42 (s, 2H), 3.52 (s, 3H), 3.05 (d, J = 5.6 Hz, 2H), 2.17 (s, 6H). |
| 86 | 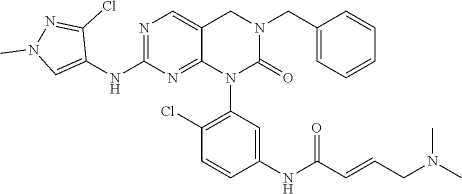 | X | 606.21 | δ 10.35 (bs, 1H), 8.82 (bs, 1H), 8.14 (s, 1H), 7.80-7.84 (m, 1H), 7.63-7.70 (m, 1H), 7.24-7.35 (m, 6H), 6.72-6.82 (m, 2H), 6.25 (d, J = 15.2 Hz, 1H), 4.71- 4.75 (m, 1H), 4.55-4.59 (m, 1H), 4.37- 4.44 (m, 2H), 3.52 (s, 3H), 3.08 (d, J = 4.8 Hz, 2H), 2.17 (s, 6H). |
| 87 | 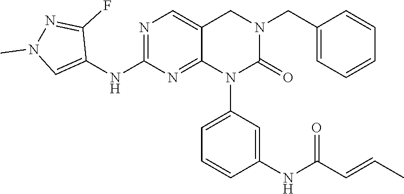 [SMILES: C/C=C/C(=O)Nc1cccc(N2C(=O)N(Cc3ccccc3)Cc3cnc(Nc4cn(C)nc4F)nc32)c1] | X | 513.23 | δ 10.15 (bs, 1H), 9.26 (bs, 1H), 8.10 (s, 1H), 7.69-7.76 (m, 1H), 7.56-7.63 (m, 1H), 7.47 (s, 1H), 7.31-7.40 (m, 5H), 7.01 (d, J = 7.6 Hz, 1H), 6.49 (s, 1H), 5.91-5.98 (m, 1H), 5.11-5.19 (m, 1H), 4.62 (s, 2H), 4.39 (s, 2H), 3.38 (s, 3H), 3.12 (d, J = 6.8 Hz, 2H), 1.85 (d, J = 7.2 Hz, 1H) (Mixture of isomers) |
| 88 | 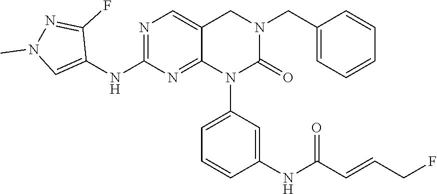 [SMILES: Cn1cc(Nc2ncc3c(n2)N(c2cccc(NC(=O)/C=C/CF)c2)C(=O)N(Cc2ccccc2)C3)c(F)n1] | X | 531.16 | δ 10.37 (s, 1H), 9.25 (bs, 1H), 8.10 (s, 1H), 7.73-7.71 (m, 1H), 7.63 (bs, 1H), 7.52-7.48 (m, 1H), 7.38-7.29 (m, 5H), 7.03 (d, J = 8.0 Hz, 1H), 6.91-6.80 (m, 1H), 6.49 (bs, 1H), 6.34 (d, J = 14.8 Hz, 1H), 5.24-5.12 (m, 2H), 4.63 (s, 2H), 4.40 (s, 2H), 3.38 (bs, 3H). |
| 89 | 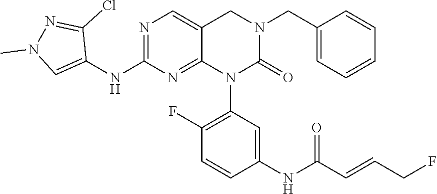 [SMILES: Cn1cc(Nc2ncc3c(n2)N(c2cc(NC(=O)/C=C/CF)ccc2F)C(=O)N(Cc2ccccc2)C3)c(Cl)n1] | X | 565.14 | δ 10.41 (s, 1H), 8.83 (bs, 1H), 8.15 (s, 1H), 7.80-7.75 (m, 2H), 7.43-7.30 (m, 6H), 6.94-6.71 (m, 2H), 6.35 (dd, J = 15.2 Hz, 1.6 Hz, 1H), 5.24-5.12 (m, 2H), 4.64 (s, 2H), 4.47-4.39 (m, 2H), 3.53 (s, 3H). |

### Scheme 29: Preparation of (E)-N-(3-(3-benzyl-7-(methylsulfonyl)-2-oxo-3,4-dihydropyrimido[4,5-d]pyrimidin-1(2H)-yl)phenyl)-4-(dimethylamino)but-2-enamide (117): Key Scaffold

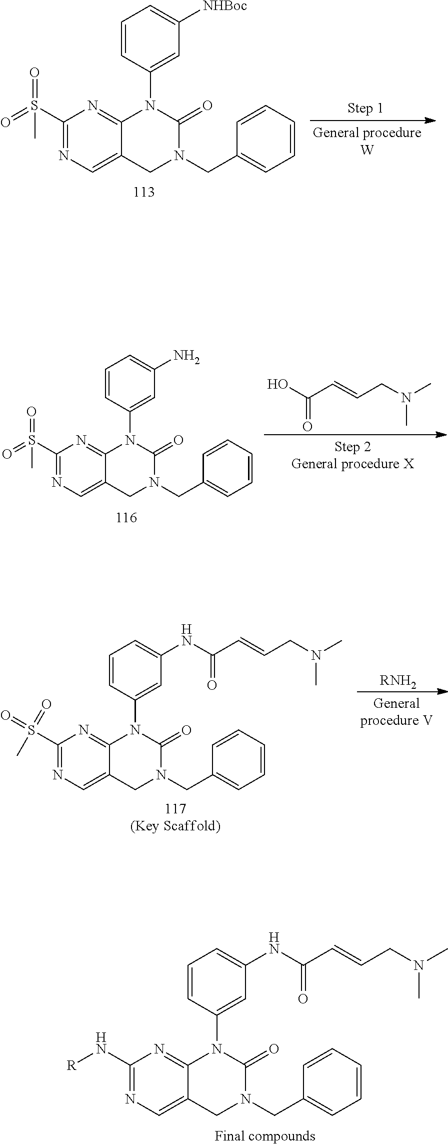

### Step 1: Preparation of 1-(3-aminophenyl)-3-benzyl-7-(methylsulfonyl)-3,4-dihydropyrimido[4,5-d]pyrimidin-2(1H)-one (116)

Title compound was prepared in a manner substantially similar to procedure mentioned in General procedure W, afforded 116 as off white solid (3.9 g; Yield: 79%). LCMS: [M+H]⁺ 410.32 (81.02% purity).

### Step 2: Preparation of (E)-N-(3-(3-benzyl-7-(methylsulfonyl)-2-oxo-3,4-dihydropyrimido[4,5-d]pyrimidin-1(2H)-yl)phenyl)-4-(dimethylamino)but-2-enamide (117)

Title compound was prepared in a manner substantially similar to procedure mentioned in General procedure X. The crude was triturated with diethyl ether afforded 117 as yellow solid (3.1 g; Yield: 36%). LCMS: [M+H]⁺ 521.42 (91.75% purity).

### Step 3: Preparation of Final Compounds

Title compound was prepared in a manner substantially similar to procedure mentioned in General procedure V. The crude product was purified using combiflash chromatography or prep-HPLC purification to get final products.

The following compounds were prepared using the methods described above

| Cmpd | | Synthesis | LCMS | |
|---|---|---|---|---|
| No. | Structure | method | [M + H]⁺ | ¹H-NMR (400 MHz, DMSO-d₆) |
| 90 | 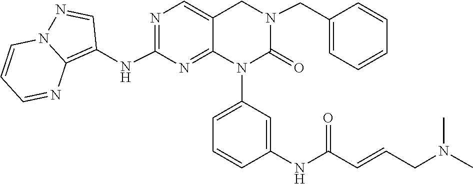 [SMILES: CN(C)C/C=C/C(=O)Nc1cccc(N2C(=O)N(Cc3ccccc3)Cc3cnc(Nc4cnn5cccnc45)nc32)c1] | V | 575.19 | δ 10.20 (s, 1H), 8.87 (m, 2H), 8.35 (d, J = 2.4 Hz, 1H), 8.10 (s, 1H), 7.69-7.65 (m, 3H), 7.41-7.32 (m, 5H), 7.32 (m, 1H), 7.01 (d, J = 8.0 Hz, 1H), 6.89-6.86 (m, 1H), 6.75-6.68 (m, 1H), 6.28 (d, J = 15.4 Hz, 1H), 4.63 (s, 2H), 4.40 (s, 2H), 3.07 (d, J = 4.0 Hz, 2H), 2.17 (s, 6H). |
| 91 | 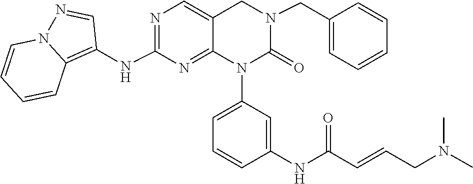 [SMILES: CN(C)C/C=C/C(=O)Nc1cccc(N2C(=O)N(Cc3ccccc3)Cc3cnc(Nc4cnn5ccccc45)nc32)c1] | V | 574.21 | δ 10.20 (s, 1H), 9.54 (s, 1H), 8.39 (d, J = 6.8 Hz, 1H), 8.12 (s, 1H), 7.82 (bs, 1H), 7.73 (s, 1H), 7.68 (d, J = 8.4 Hz, 1H), 7.44-7.29 (m, 7H), 7.04 (d, J = 8.0 Hz, 1H), 6.98-6.94 (t, J = 8.4 Hz, 1H), 6.75-6.68 (m, 2H), 6.28 (d, J = 16.5 Hz, 1H), 4.64 (s, 2H), 4.41 (s, 2H), 3.04 (d, J = 5.6 Hz, 2H), 2.15 (s, 6H). |
| 92 | 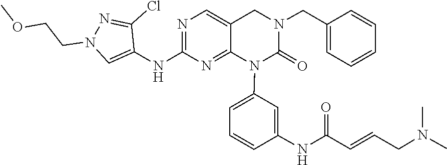 | V | 616.28 | δ 10.23 (s, 1H), 8.76 (bs, 1H), 8.12 (s, 1H), 7.74 (d, J = 8.0 Hz, 1H), 7.64 (s, 1H), 7.44 (m, 1H), 7.35 (m, 4H), 7.28 (m, 1H), 7.02 (d, J = 8.8 Hz, 1H), 6.70 (m, 2H), 6.24 (d, J = 15.2 Hz, 1H), 4.63 (s, 2H), 4.40 (s, 2H), 3.84 (s, 2H), 3.46 (s, 2H), 3.14 (s, 3H), 3.04 (d, J = 4.8 Hz, 2H), 2.16 (s, 6H). |
| 93 | 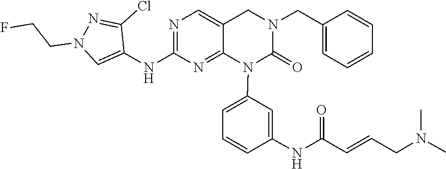 [SMILES: CN(C)C/C=C/C(=O)Nc1cccc(N2C(=O)N(Cc3ccccc3)Cc3cnc(Nc4cn(CCF)nc4Cl)nc32)c1] | V | 604.21 | δ 10.21 (s, 1H), 8.67 (bs, 1H), 8.12 (s, 1H), 7.76 (d, J = 7.6 Hz, 1H), 7.62 (s, 1H), 7.40 (m, 1H), 7.31 (m, 4H), 7.29 (m, 1H), 7.02 (d, J = 7.6 Hz, 1H), 6.70 (m, 2H), 6.24 (d, J = 15.2 Hz, 1H), 4.63 (s, 3H), 4.49 (s, 1H), 4.40 (s, 2H), 4.00 (m, 2H), 3.08 (d, J = 4.8 Hz, 2H), 2.19 (s, 6H). |
| 94 | 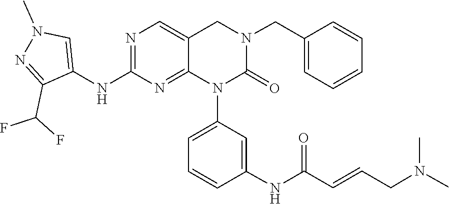 [SMILES: CN(C)C/C=C/C(=O)Nc1cccc(N2C(=O)N(Cc3ccccc3)Cc3cnc(Nc4cn(C)nc4C(F)F)nc32)c1] | V | 588.26 | δ 10.26 (s, 1H), 9.03 (bs, 1H), 8.14 (s, 1H), 7.78 (m, 1H), 7.67 (s, 1H), 7.51 (m, 1H), 7.29 (m, 5H), 7.05 (m, 2H), 6.70 (m, 2H), 6.24 (d, J = 15.6 Hz, 1H), 4.64 (s, 2H), 4.42 (s, 2H), 3.54 (s, 3H), 3.04 (d, J = 5.2 Hz, 2H), 2.16 (s, 6H). |
| 95 | 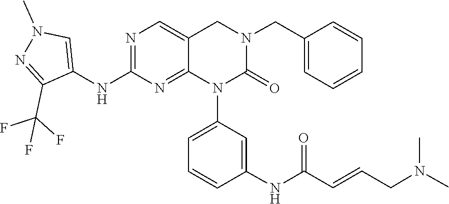 [SMILES: CN(C)C/C=C/C(=O)Nc1cccc(N2C(=O)N(Cc3ccccc3)Cc3cnc(Nc4cn(C)nc4C(F)(F)F)nc32)c1] | V | 606.26 | δ 10.35 (s, 1H), 8.51 (bs, 1H), 8.13 (s, 1H), 7.76 (d, J = 8.0 Hz, 1H), 7.65 (s, 1H), 7.48 (m, 1H), 7.35 (m, 4H), 7.29 (m, 1H), 6.92 (m, 2H), 6.71 (m, 1H), 6.32 (d, J = 15.6 Hz, 1H), 4.63 (s, 2H), 4.42 (s, 2H), 3.61 (s, 3H), 3.38 (s, 2H), 2.40 (s, 6H). |
| 96 | 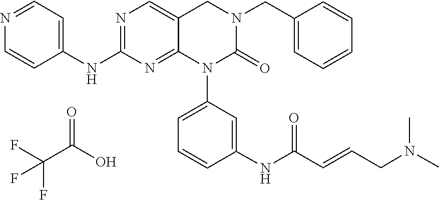 [SMILES: CN(C)C/C=C/C(=O)Nc1cccc(N2C(=O)N(Cc3ccccc3)Cc3cnc(Nc4ccncc4)nc32)c1 ; O=C(O)C(F)(F)F] | V | 535.26 | δ 10.52 (bs, 1H), 9.84 (bs, 1H), 8.91 (bs, 1H), 8.67 (d, J = 8.0 Hz, 2H), 8.61 (s, 1H), 7.77 (s, 1H), 7.66 (m, 1H), 7.50 (t, J = 8.0 Hz, 1H), 7.40 (m, 4H), 7.31 (m, 1H), 7.13 (m, 1H), 6.87 (m, 2H), 6.70 (m, 1H), 6.45 (d, J = 15.2 Hz, 1H), 4.67 (s, 2H), 4.63 (s, 2H), 3.93 (s, 2H), 2.79 (s, 6H). |
| 97 | 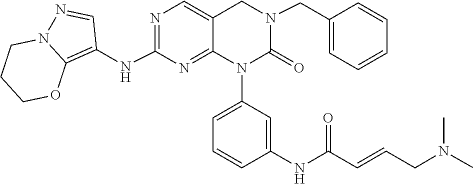 [SMILES: CN(C)C/C=C/C(=O)Nc1cccc(N2C(=O)N(Cc3ccccc3)Cc3cnc(Nc4cnn5c4OCCC5)nc32)c1] | V | 580.34 | δ 10.15 (s, 1H), 8.28 (bs, 1H), 7.97 (s, 1H), 7.60 (s, 2H), 7.28 (m, 6H), 6.93 (d, J = 7.6 Hz, 2H), 6.69 (m, 1H), 6.24 (d, J = 15.2 Hz, 1H), 4.60 (s, 2H), 4.33 (s, 2H), 4.13 (s, 2H), 3.94 (s, 2H), 3.04 (m, 2H), 2.17 (s, 6H), 2.06 (s, 2H). |
| 98 | 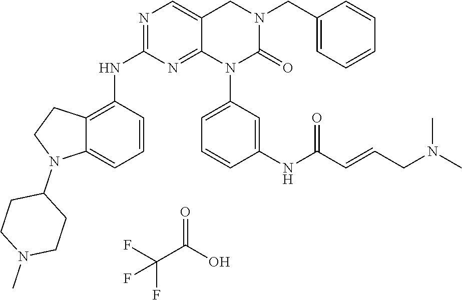 [SMILES: CN(C)C/C=C/C(=O)Nc1cccc(N2C(=O)N(Cc3ccccc3)Cc3cnc(Nc4cccc5c4CCN5C4CCN(C)CC4)nc32)c1 ; O=C(O)C(F)(F)F] | V | 672.42 | δ 10.46 (bs, 1H), 9.85 (bs, 1H), 9.39 (s, 1H), 8.44 (s, 1H), 8.11 (s, 1H), 7.72 (m, 1H), 7.62 (s, 1H), 7.37 (m, 5H), 7.03 (d, J = 8.0 Hz, 1H), 6.69 (m, 1H), 6.63 (m, 1H), 6.55 (m, 1H), 6.43 (d, J = 15.2 Hz, 1H), 6.07 (d, J = 6.8 Hz, 1H), 4.63 (m, 2H), 4.40 (m, 2H), 3.94 (m, 2H), 3.46 (m, 3H), 3.17 (m, 2H), 3.03 (m, 2H), 2.77 (m, 11H), 1.72 (m, 4H). |
| 99 | 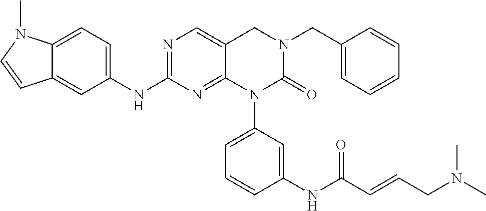 [SMILES: CN(C)C/C=C/C(=O)Nc1cccc(N2C(=O)N(Cc3ccccc3)Cc3cnc(Nc4ccc5c(ccn5C)c4)nc32)c1] | V | 586.92 | δ 10.24 (bs, 1H), 9.21 (bs, 1H), 8.12 (s, 1H), 7.83 (d, J = 8.8 Hz, 1H), 7.68 (s, 1H), 7.30 (m, 7H), 7.12 (m, 1H), 7.03 (m, 3H), 6.70 (m, 1H), 6.23 (d, J = 15.6 Hz, 1H), 6.07 (s, 1H), 4.64 (m, 2H), 4.40 (m, 2H), 3.66 (s, 3H), 3.02 (d, J = 5.2 Hz, 2H), 2.14 (s, 6H). |
| 100 | 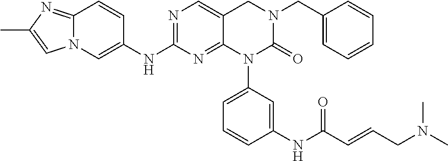 | V | 588.27 | δ 10.33 (bs, 1H), 9.53 (bs, 1H), 8.20 (s, 2H), 7.98 (d, J = 7.6 Hz, 1H), 7.67 (m, 1H), 7.52 (s, 1H), 7.33 (m, 4H), 7.29 (m, 1H), 7.19 (d, J = 5.6 Hz, 1H), 7.16 (m, 2H), 7.08 (m, 1H), 6.70 (m, 1H), 6.24 (d, J = 15.6 Hz, 1H), 4.64 (s, 2H), 4.44 (s, 2H), 3.05 (d, J = 5.6 Hz, 2H), 2.24 (s, 3H), 2.16 (s, 6H). |
| 101 | 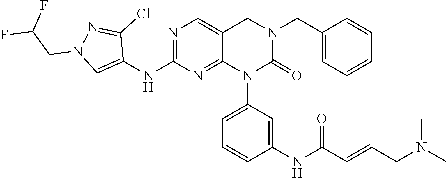 [SMILES: CN(C)C/C=C/C(=O)Nc1cccc(N2C(=O)N(Cc3ccccc3)Cc3cnc(Nc4cn(CC(F)F)nc4Cl)nc32)c1] | V | 622.28 | δ 10.22 (bs, 1H), 8.79 (bs, 1H), 8.13 (s, 1H), 7.74 (d, J = 8.4 Hz, 1H), 7.63 (s, 1H), 7.47-7.29 (m, 7H), 7.03 (d, J = 7.92 Hz, 1H), 6.76-6.69 (m, 1H), 6.27-6.02 (m, 2H), 4.63 (s, 2H), 4.41 (s, 2H), 4.19 (m, 2H), 3.05-3.04 (m, 2H), 2.16 (s, 6H). |
| 102 | 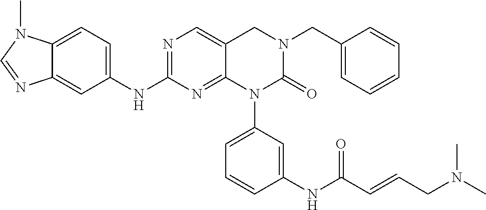 [SMILES: CN(C)C/C=C/C(=O)Nc1cccc(N2C(=O)N(Cc3ccccc3)Cc3cnc(Nc4ccc5c(c4)ncn5C)nc32)c1] | V | 588.34 | δ 10.21 (bs, 1H), 9.75 (m, 1H), 8.20 (s, 1H), 7.81 (s, 1H), 7.77-7.75 (m, 1H), 7.67-7.61 (m, 3H), 7.49 (t, J = 8.04 Hz, 1H), 7.41-7.30 (m, 5H), 7.09 (d, J = 8.7 Hz, 1H), 6.76-6.70 (m, 1H), 6.50-6.41 (m, 2H), 4.64 (s, 2H), 4.44 (s, 2H), 3.94-3.92 (m, 2H), 3.86 (s, 3H), 2.79 (s, 6H). |
| 103 | 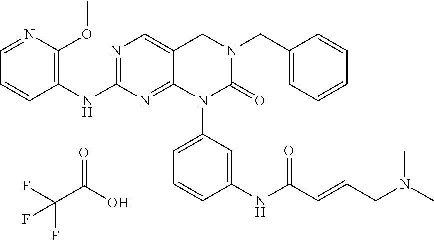 [SMILES: COc1ncccc1Nc1ncc2c(n1)N(c1cccc(NC(=O)/C=C/CN(C)C)c1)C(=O)N(Cc1ccccc1)C2 ; O=C(O)C(F)(F)F] | V | 565.39 | δ 10.49 (s, 1H), 9.75 (m, 1H), 8.20 (s, 1H), 7.81 (s, 1H), 7.77-7.75 (m, 1H), 7.67-7.61 (m, 3H), 7.49 (t, J = 8.0 Hz, 1H), 7.41-7.30 (m, 5H), 7.09 (d, J = 8.7 Hz, 1H), 6.76-6.70 (m, 1H), 6.50-6.41 (m, 2H), 4.64 (s, 2H), 4.44 (s, 2H), 3.94-3.92 (m, 2H), 3.86 (s, 3H), 2.79 (s, 6H). |

### Scheme 30: Alternative Preparation of (E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(dimethylamino)but-2-enamide (Compound 35)

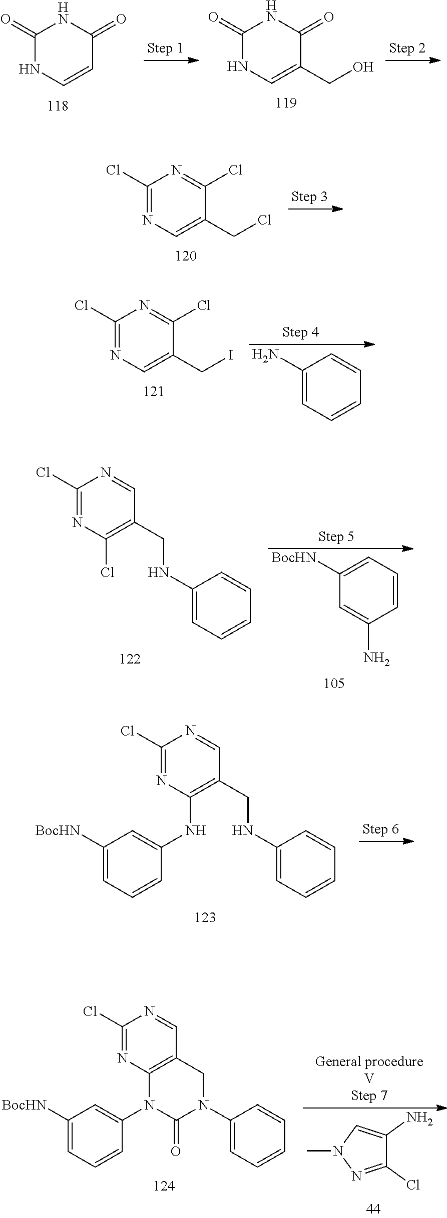

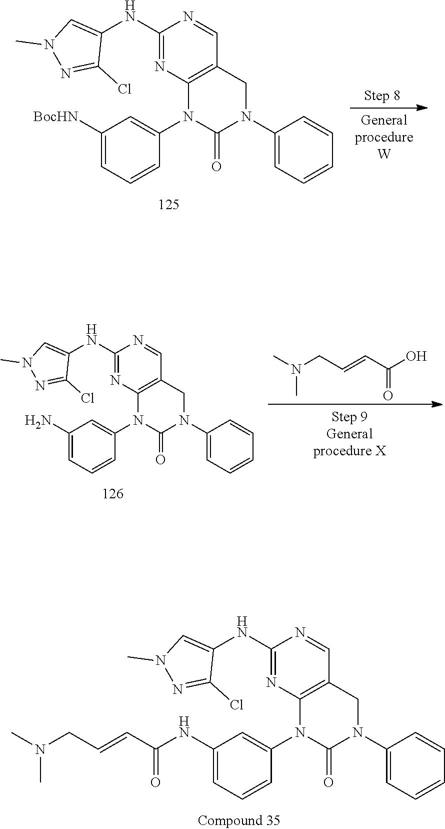

### Step 1: Preparation of 5-(hydroxymethyl)pyrimidine-2,4(1H,3H)-dione (119)

An ice-cold solution of pyrimidine-2,4(1H,3H)-dione (118) (10 g, 89.21 mmol) and paraformaldehyde (9.63 g, 107.05 mmol) in aqueous potassium hydroxide (132 mL, 0.5 M, 66.74 mmol) was heated at 55° C. for 14 hours. After completion of starting material (TLC), the reaction mixture was cooled to 0° C. and the pH was adjusted to 6 with 12N hydrochloric acid, the resulting white precipitate was filtered through sintered funnel and washed with diethyl ether afforded 119 as a white solid (6.3 g, Yield: 50%) which was used directly for the next step. ¹H-NMR (400 MHz, DMSO-d₆): δ 10.98 (bs, 1H), 10.64 (bs, 1H), 7.24 (s, 1H), 4.78 (m, 1H), 4.12 (d, J=12.8 Hz, 2H). LCMS: [M+H]⁺ 143.04 (99.92% purity).

### Step 2: Preparation of 2,4-dichloro-5-(chloromethyl)pyrimidine (120)

To an ice-cold solution of 5-(hydroxymethyl)pyrimidine-2,4(1H,3H)-dione (119) (10 g, 70.36 mmol) in toluene (25 mL) was added phosphoryl chloride (14 mL, 140.72 mmol) then N,N-diisopropylethylamine (37 mL, 211 mmol). The reaction mixture was heated at 120° C. for 16 hours. After the complete disappearance of starting material on TLC, the reaction mixture was quenched slowly with sodium bicarbonate solution and extracted with ethyl acetate (3×200 mL). The combined organic layer was washed with brine, dried over anhydrous sodium sulfate, filtered and evaporated under reduced pressure afforded 120 as a brown solid (12 g, Yield: 86%) which was used directly for the next step. ¹H NMR (400 MHz, CDCl₃): δ 8.66 (s, 1H), 4.64 (s, 2H). MS: [M+H]⁺ 197.0

### Step 3: Preparation of 2,4-dichloro-5-(iodomethyl)pyrimidine (121)

To a solution of 2,4-dichloro-5-(chloromethyl)pyrimidine (120) (8.0 g, 40.51 mmol in acetone (40 mL) was added sodium iodide (9.71 g, 64.82 mmol). The reaction mixture was stirred at room temperature for 30 min and heated to reflux for 2 hours. After completion of reaction (TLC monitoring), the reaction mixture cooled to room temperature. The resulting white precipitate was filtered through sintered funnel and washed with acetone. The filtrate was concentrated under reduced pressure afforded 121 as a brown solid (10 g, Yield: 85%) which was used directly for the next step. ¹H-NMR (400 MHz, CDCl₃): δ 8.60 (s, 1H), 4.39 (s, 2H).

### Step 4: Preparation of N-((2,4-dichloropyrimidin-5-yl)methyl)aniline (122)

To an ice-cold solution of 2, 4-dichloro-5-(iodomethyl)pyrimidine (121) (5.0 g, 17.30 mmol) in acetone (50 mL) was added potassium carbonate (5.26 g, 38.06 mmol) and aniline (1.93 g, 20.76 mmol). The resulting reaction mixture was stirred at room temperature for 16 hours. After completion the reaction (as per TLC monitoring), the resulting white precipitate was filtered through sintered funnel and washed with acetone. The filtrate was concentrated under reduced pressure and crude was purified by column chromatography on silica gel (100-200 mesh) using 15% ethyl acetate-hexane as an eluent afforded 122 as a brown solid (2.5 g, Yield: 57%). ¹H-NMR (400 MHz, CDCl₃): δ 8.61 (s, 1H), 7.07 (t, J=7.6 Hz, 2H), 6.58 (m, 3H), 6.30 (bs, 1H), 4.33 (m, 2H). LCMS: [M+H]⁺ 254.03 (99.01% purity).

### Step 5: Preparation of tert-butyl (3-(7-chloro-2-oxo-3-phenyl-3,4-dihydropyrimido[4,5-d]pyrimidin-1(2H)-yl)phenyl)carbamate (123)

To an ice-cold solution of N-((2,4-dichloropyrimidin-5-yl)methyl)aniline (122) (500 mg, 1.96 mmol), in isopropanol (5 mL) was added N,N-diisopropylethylamine (1.47 mL, 8.42 mmol) and tert-butyl (3-aminophenyl)carbamate (105) (409 mg, 1.96 mmol). The resulting reaction mixture was heated at 100° C. for 16 hours in a sealed tube. After completion of reaction (TLC monitoring), the solvent was then evaporated under reduced pressure and resulting crude was purified by column chromatography on silica gel (100-200 mesh) using 30% ethyl acetate-hexane as an eluent afforded 123 as a brown solid (500 mg, Yield: 60%). ¹H-NMR (400 MHz, DMSO-d₆): δ 9.41 (s, 1H), 8.96 (s, 1H), 8.10 (s, 1H), 7.73 (s, 1H), 7.25 (m, 2H), 7.12 (m, 3H), 6.61 (m, 3H), 6.14 (t, J=7.2 Hz, 1H), 4.26 (m, 2H) and 1.53 (s, 9H). LCMS: [M+H]⁺ 426.14 (93% purity).

### Step 6: Preparation of tert-butyl (3-(7-chloro-2-oxo-3-phenyl-3,4-dihydropyrimido[4,5-d]pyrimidin-1(2H)-yl)phenyl)carbamate (124)

To an ice-cold solution of tert-butyl (3-(7-chloro-2-oxo-3-phenyl-3,4-dihydropyrimido[4,5-d]pyrimidin-1(2H)-yl)phenyl)carbamate (123) (500 mg, 1.17 mmol) in tetrahydrofuran (6 mL) was added N,N-diisopropylethylamine (0.81 ml, 4.68 mmol) and triphosgene (139 mg, 0.46 mmol). The reaction mixture was stirred at room temperature for 3 hours. After completion of the reaction (TLC monitoring), aqueous triethylamine solution was added and extracted with dichloromethane (3 times). The combined organic layer was washed with brine and dried over sodium sulfate and evaporated under reduced pressure to obtain the crude residue. The crude was purified by column chromatography on silica gel (100-200 mesh) using 30% ethyl acetate-hexane as an eluent afforded 124 as a brown solid (450 mg, Yield: 85%). ¹H-NMR (400 MHz, DMSO-d₆): δ 9.54 (s, 1H), 8.43 (s, 1H), 7.58 (s, 1H), 7.44 (m, 4H), 7.29 (t, J=7.2 Hz, 3H), 6.94 (s, 1H), 5.0 (s, 2H) and 1.47 (s, 9H). LCMS: [M+H]⁺ 452.27 (99% purity).

### Step 7: Preparation of tert-butyl (3-(7-((3-chloro-1-methyl-1H-pyrazol-4-yl)amino)-2-oxo-3-phenyl-3,4-dihydropyrimido[4,5-d]pyrimidin-1(2H)-yl)phenyl)carbamate (125)

Title compound was prepared in a manner substantially similar to procedure mentioned in General procedure V, (tert-butyl(3-(7-chloro-2-oxo-3-phenyl-3,4-dihydropyrimido[4,5-d]pyrimidin-1(2H)-yl)phenyl)carbamate (124) and 3-chloro-1-methyl-1H-pyrazol-4-amine (44) gave tert-butyl (3-(7-((3-chloro-1-methyl-1H-pyrazol-4-yl)amino)-2-oxo-3-phenyl-3,4-dihydropyrimido[4,5-d]pyrimidin-1(2H)-yl)phenyl)carbamate (125) as a brown solid in 70% yield, which was used directly for the next step. MS: [M+H]⁺ 547.17.

### Step 8: Preparation of 1-(3-aminophenyl)-7-((3-chloro-1-methyl-1H-pyrazol-4-yl)amino)-3-phenyl-3,4-dihydropyrimido[4,5-d]pyrimidin-2(1H)-one (126)

Title compound was prepared in a manner substantially similar to procedure mentioned in General procedure W, tert-butyl (3-(7-((3-chloro-1-methyl-1H-pyrazol-4-yl)amino)-2-oxo-3-phenyl-3,4-dihydropyrimido[4,5-d]pyrimidin-1(2H)-yl)phenyl)carbamate (125) gave 1-(3-aminophenyl)-7-((3-chloro-1-methyl-1H-pyrazol-4-yl)amino)-3-phenyl-3,4-dihydropyrimido[4,5-d]pyrimidin-2(1H)-one (126) as a brown solid (800 mg, Yield: 82%) which was used directly for the next step. MS: [M+H]⁺ 447.08.

### Step 9: Preparation of (E)-N-(3-(7-((3-chloro-1-methyl-1H-pyrazol-4-yl)amino)-2-oxo-3-phenyl-3,4-dihydropyrimido[4,5-d]pyrimidin-1(2H)-yl)phenyl)-4-(dimethylamino)but-2-enamide (Compound 35)

Title compound was prepared in a manner substantially similar to procedure mentioned in General procedure X, 1-(3-aminophenyl)-7-((3-chloro-1-methyl-1H-pyrazol-4-yl)amino)-3-phenyl-3,4-dihydropyrimido[4,5-d]pyrimidin-2(1H)-one (126) and trans-N,N-dimethylaminocrotonic acid hydrochloride gave the titled compound, which was purified by prep-HPLC purification to afforded the title compound Compound 35 as a white solid (285 mg, Yield: 23%). ¹H-NMR (400 MHz, DMSO-d₆): δ 10.27 (bs, 1H), 8.86 (s, 1H), 8.21 (s, 1H), 7.73 (s, 2H), 7.51-7.40 (m, 5H), 7.30-7.25 (m, 1H), 7.09 (d, J=7.6 Hz, 1H), 6.76-6.70 (m, 2H), 6.29 (d, J=15.4 Hz, 1H), 4.88 (s, 2H), 3.50 (s, 3H), 3.05 (d, J=4.8 Hz, 2H) and 2.16 (s, 6H). MS: [M+H]⁺ 558.16.

The following compounds were prepared using the methods described above

| Cmpd | | Synthesis | LCMS | |
|---|---|---|---|---|
| No. | Structure | method | [M + H]⁺ | ¹H-NMR (400 MHz, DMSO-d₆) |
| 104 | 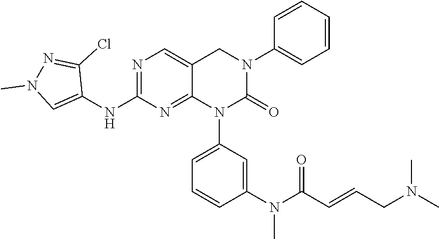 | X | 572.26 | δ 8.31 (s, 1H), 8.20 (s, 1H), 7.64 (t, J = 7.2 Hz, 1H), 7.46-7.38 (m, 6H), 7.30-7.26 (m, 2H), 7.01 (s, 1H), 6.64- 6.57 (m, 1H), 5.99 (d, J = 15.2 Hz, 1H), 4.87 (s, 2H), 3.56 (s, 3H), 3.27 (s, 3H), 2.73 (d, J = 4.8 Hz, 2H), 2.01 (s, 6H). |
| 105 | 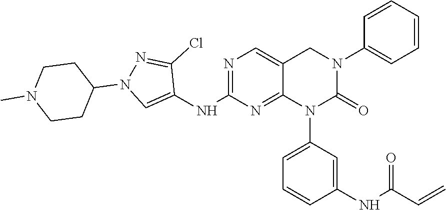 [SMILES: C=CC(=O)Nc1cccc(N2C(=O)N(c3ccccc3)Cc3cnc(Nc4cn(C5CCN(C)CC5)nc4Cl)nc32)c1] | Y | 584.30 | δ 10.34 (bs, 1H), 8.80 (bs, 1H), 8.22 (s, 1H), 7.78 (d, J = 7.2 Hz, 1H), 7.69 (s, 1H), 7.40 (m, 5H), 7.26 (m, 1H), 7.08 (m, 1H), 6.91 (s, 1H), 6.40 (m, 1H), 6.24 (d, J = 16.4 Hz, 1H), 5.76 (d, J = 10.4 Hz, 1H), 4.88 (s, 2H), 3.50 (s, 1H), 2.80 (m, 2H), 2.22 (s, 3H), 2.03 (m, 2H), 1.74 (m, 2H), 1.93 (m, 2H). |
| 106 | 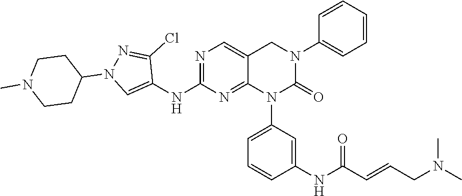 | X | 641.37 | δ 10.27 (bs, 1H), 8.83 (bs, 1H), 8.22 (s, 1H), 7.75 (d, J = 8.0 Hz, 1H), 7.69 (s, 1H), 7.50-7.40 (m, 5H), 7.30-7.20 (m, 1H), 7.07 (d, J = 8.0 Hz, 1H), 6.91 (bs, 1H), 6.77-6.70 (m, 1H), 6.27 (d, J = 15.2 Hz, 1H), 4.88 (s, 2H), 3.50 (bs, 1H), 3.05 (d, J = 5.2 Hz, 2H), 2.81 (d, J = 11.2 Hz, 2H), 2.21 (s, 3H), 2.16 (s, 6H), 2.05-1.60 (m, 6H). |
| 107 | 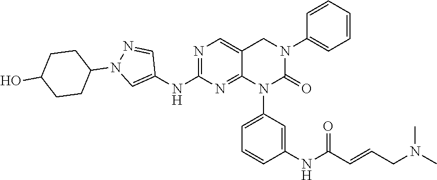 [SMILES: CN(C)C/C=C/C(=O)Nc1cccc(N2C(=O)N(c3ccccc3)Cc3cnc(Nc4cnn(C5CCC(O)CC5)c4)nc32)c1] | X | 608.42 | δ 10.30 (bs, 1H), 8.17 (s, 1H), 7.81- 7.70 (m, 2H), 7.52-7.40 (m, 5H), 7.29- 7.25 (m, 1H), 7.10-7.08 (m, 2H), 6.67- 6.70 (m, 2H), 6.27 (d, J = 15.2 Hz, 1H), 4.86 (s, 2H), 4.60 (m, 1H), 3.57- 3.42 (m, 2H), 3.05 (d, J = 5.6 Hz, 2H), 2.16 (s, 6H), 1.88-1.85 (m, 2H), 11.30 (s, 7H), |
| 108 | 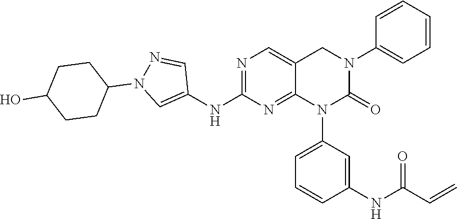 [SMILES: C=CC(=O)Nc1cccc(N2C(=O)N(c3ccccc3)Cc3cnc(Nc4cnn(C5CCC(O)CC5)c4)nc32)c1] | Y | 551.33 | δ 10.38 (bs, 1H), 9.54 (s, 1H), 8.17 (s, 1H), 7.82 (d, J = 8.0 Hz, 1H), 7.74 (s, 1H), 7.52 (t, J = 8.0 Hz, 1H), 7.40 (m, 4H), 7.28-7.25 (m, 1H), 7.12-7.08 (m, 2H), 6.74 (s, 1H), 6.47-6.40 (m, 1H), 6.26 (d, J = 18.4 Hz, 1H), 5.76 (d, J = 11.6 Hz, 1H), 4.86 (s, 2H), 4.66 (d, J = 3.6 Hz, 1H), 3.60-358 (m, 1H), 3.49-3.42 (m, 1H), 1.87 (d, J = 10.4 Hz, 2H) 1.61-1.50 (m, 4H), 1.38-1.30 (m, 2H). |
| 109 | 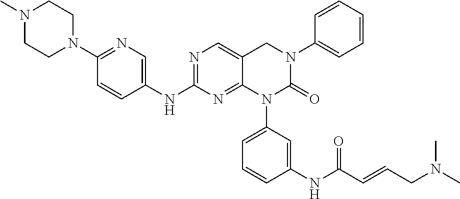 | X | 619.45 | δ 10.20 (bs, 1H), 9.23 (bs, 1H), 8.18 (s, 1H), 8.03 (bs, 1H), 7.75 (d, J = 8.4 Hz, 1H), 7.66 (s, 1H), 7.41-7.46 (m, 6H), 7.26-7.29 (m, 1H), 7.03 (d, J = 7.2 Hz, 1H), 6.69-6.76 (m, 1H), 6.36 (m, 1H), 6.24 (d, J = 15.6 Hz, 1H), 4.87 (s, 2H), 3.29 (m, 4H), 3.04 (d, J = 5.6 Hz, 2H), 2.34 (m, 4H), 2.20 (s, 3H), 2.16 (s, 6H). |
| 110 | 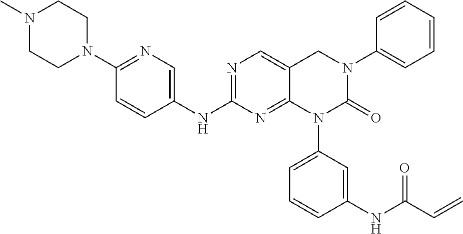 [SMILES: C=CC(=O)Nc1cccc(N2C(=O)N(c3ccccc3)Cc3cnc(Nc4ccc(N5CCN(C)CC5)nc4)nc32)c1] | Y | 562.34 | δ 10.30 (bs, 1H), 9.23 (bs, 1H), 8.18 (s, 1H), 8.04 (bs, 1H), 7.77 (d, J = 8.4 Hz, 1H), 7.66 (s, 1H), 7.40-7.51 (m, 6H), 7.26-7.29 (m, 1H), 7.06 (d, J = 8.0 Hz, 1H), 6.40-6.47 (m, 2H), 6.23 (d, J = 17.2 Hz, 1H), 5.74 (d, J = 11.6 Hz, 1H), 4.87 (s, 2H), 3.29 (m, 4H), 2.34-2.37 (m, 4H), 2.19 (s, 3H). |
| 111 | 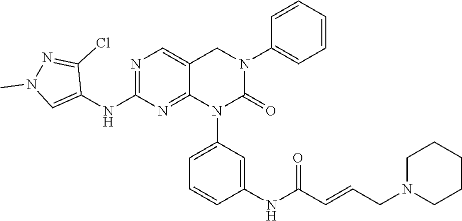 [SMILES: Cn1cc(Nc2ncc3c(n2)N(c2cccc(NC(=O)/C=C/CN4CCCCC4)c2)C(=O)N(c2ccccc2)C3)c(Cl)n1] | X | 598.36 | δ 10.25 (bs, 1H), 8.84 (bs, 1H), 8.21 (s, 1H), 7.72 (d, J = 8.0 Hz, 2H), 7.40- 7.51 (m, 5H), 7.26-7.29 (m, 1H), 7.07 (d, J = 8.0 Hz, 1H), 6.70-6.77 (m, 2H), 6.23 (d, J = 15.2 Hz, 1H), 4.88 (s, 2H), 3.50 (s, 3H), 3.06 (d, J = 5.2 Hz, 2H), 2.33 (s, 4H), 1.49-1.52 (m, 4H), 1.37 (s, 2H). |
| 112 | 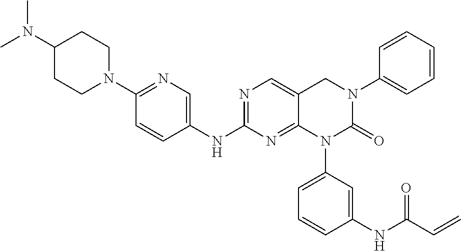 [SMILES: C=CC(=O)Nc1cccc(N2C(=O)N(c3ccccc3)Cc3cnc(Nc4ccc(N5CCC(N(C)C)CC5)nc4)nc32)c1] | Y | 590.51 | δ 10.29 (bs, 1H), 9.15 (bs, 1H), 8.09 (s, 1H), 7.99 (s, 1H), 7.80 (d, J = 8.0 Hz, 1H), 7.58 (s, 1H), 7.31-7.44 (m, 7H), 7.01 (d, J = 8.0 Hz, 1H), 6.35- 6.46 (m, 2H), 6.23-6.27 (m, 1H), 5.75 (d, J = 10.0 Hz, 1H), 4.87 (s, 2H), 4.06-4.09 (m, 2H), 2.61-2.67 (m, 2H), 2.17-2.24 (m, 7H), 1.74-1.77 (m, 2H), 1.25-1.33 (m, 2H). |
| 113 | 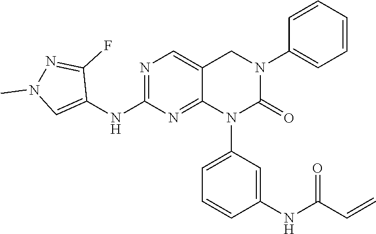 [SMILES: C=CC(=O)Nc1cccc(N2C(=O)N(c3ccccc3)Cc3cnc(Nc4cn(C)nc4F)nc32)c1] | Y | 485.20 | δ 10.35 (bs, 1H), 8.19 (s, 1H), 7.72- 7.78 (m, 2H), 7.41-7.49 (m, 6H), 7.26- 7.29 (m, 1H), 7.09 (d, J = 8.4 Hz, 1H), 6.40-6.53 (m, 2H), 6.23-6.28 (m, 1H), 5.75 (d, J = 10.4 Hz, 1H), 4.87 (s, 2H), 3.29 (s, 3H). |
| 114 | 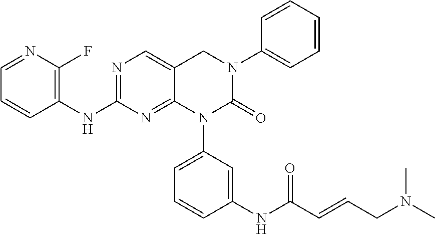 [SMILES: CN(C)C/C=C/C(=O)Nc1cccc(N2C(=O)N(c3ccccc3)Cc3cnc(Nc4cccnc4F)nc32)c1] | X | 539.49 | δ 10.21 (bs, 1H), 9.11 (bs, 1H), 8.27 (s, 1H), 7.87 (d, J = 8.4 Hz, 1H), 7.74 (s, 1H), 7.65-7.68 (m, 2H), 7.41-7.45 (m, 5H), 7.28 (s, 1H), 7.06 (d, J = 7.6 Hz, 1H), 6.85 (s, 1H), 6.69-6.74 (m, 1H), 6.24 (d, J = 15.6 Hz, 1H), 4.92 (s, 2H), 3.05 (d, J = 5.2 Hz, 2H), 2.17 (s, 6H). |
| 115 | 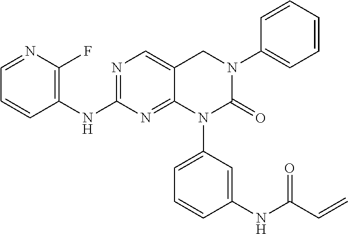 [SMILES: C=CC(=O)Nc1cccc(N2C(=O)N(c3ccccc3)Cc3cnc(Nc4cccnc4F)nc32)c1] | Y | 482.30 | δ 10.29 (bs, 1H), 9.11 (bs, 1H), 8.27 (s, 1H), 7.87 (d, J = 8.4 Hz, 1H), 7.68- 7.74 (m, 3H), 7.41-7.47 (m, 5H), 7.28 (s, 1H), 7.08 (d, J = 7.6 Hz, 1H), 6.85 (s, 1H), 6.40-6.46 (m, 1H), 6.23-6.27 (m, 1H), 5.74 (d, J = 10.4 Hz, 1H), 4.92 (s, 2H). |
| 116 | 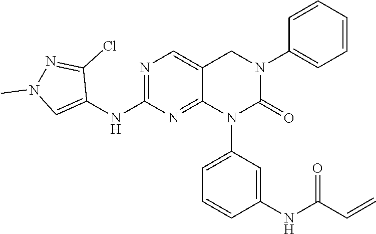 [SMILES: C=CC(=O)Nc1cccc(N2C(=O)N(c3ccccc3)Cc3cnc(Nc4cn(C)nc4Cl)nc32)c1] | Y | 501.35 | δ 10.34 (bs, 1H), 8.82 (bs, 1H), 8.21 (s, 1H), 7.72-7.77 (m, 2H), 7.43-7.53 (m, 5H), 7.28-7.30 (m, 1H), 7.10 (d, J = 7.6 Hz, 1H), 6.74 (s, 1H), 6.40-6.47 (m, 1H), 6.23-6.28 (m, 1H), 5.75 (d, J = 10.0 Hz, 1H), 4.88 (s, 2H), 3.50 (s, 3H). |
| 117 | 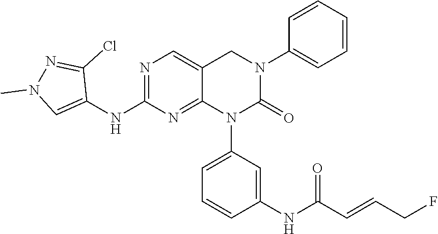 [SMILES: Cn1cc(Nc2ncc3c(n2)N(c2cccc(NC(=O)/C=C/CF)c2)C(=O)N(c2ccccc2)C3)c(Cl)n1] | X | 533.37 | δ 10.15 (bs, 1H), 8.28 (bs, 1H), 8.19 (s, 1H), 7.68-7.76 (m, 2H), 7.40-7.51 (m, 5H), 7.26-7.29 (m, 1H), 7.06-7.10 (m, 1H), 6.83-6.94 (m, 2H), 6.36-6.66 (m, 1H), 5.16-5.21 (m, 1H), 5.05-5.09 (m, 1H), 4.88 (s, 2H), 3.54 (s, 3H). |
| 118 | 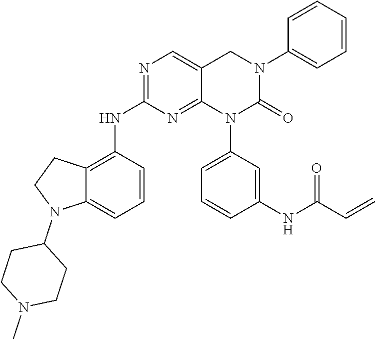 [SMILES: C=CC(=O)Nc1cccc(N2C(=O)N(c3ccccc3)Cc3cnc(Nc4cccc5c4CCN5C4CCN(C)CC4)nc32)c1] | Y | 601.48 | δ 10.27 (bs, 1H), 8.42 (bs, 1H), 8.26 (s, 1H), 7.66-7.71 (m, 2H), 7.39-7.47 (m, 5H), 7.25-7.28 (m, 1H), 7.03 (d, J = 8.0 Hz, 1H), 6.53-6.62 (m, 2H), 6.40-6.47 (m, 1H), 6.23-6.27 (m, 1H), 6.0 (d, J = 7.6 Hz, 1H), 5.74 (d, J = 11.6 Hz, 1H), 4.87 (s, 2H), 3.18-3.20 (m, 3H), 2.73-2.82 (m, 4H), 2.22 (s, 3H), 1.90-1.94 (m, 2H), 1.56-1.57 (m, 4H). |
| 119 | 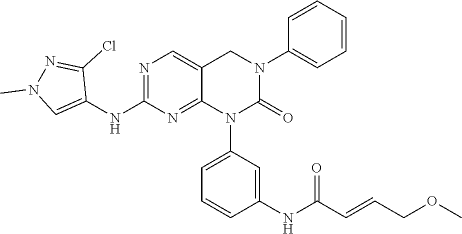 | X | 545.36 | δ 10.26 (bs, 1H), 8.76 (bs, 1H), 8.20 (s, 1H), 7.73 (s, 2H), 7.30-7.44 (m, 5H), 7.25-7.30 (m, 1H), 7.07 (d, J = 8.0 Hz, 1H), 6.77-6.83 (m, 2H), 6.30 (d, J = 13.2 Hz, 1H), 4.88 (s, 2H), 4.09-4.11 (m, 2H), 3.51 (s, 3H), 3.29 (s, 3H). |
| 120 | 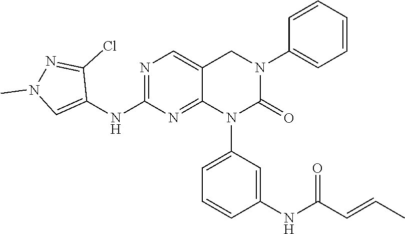 [SMILES: C/C=C/C(=O)Nc1cccc(N2C(=O)N(c3ccccc3)Cc3cnc(Nc4cn(C)nc4Cl)nc32)c1] | X | 515.28 | δ 10.12 (bs, 1H), 8.74 (bs, 1H), 8.20 (s, 1H), 7.63 (s, 2H), 7.39-7.50 (m, 5H), 7.21-7.24 (m, 1H), 7.06 (d, J = 8.4 Hz, 1H), 6.75-6.84 (m, 2H), 6.14 (d, J = 15.2 Hz, 1H), 4.88 (s, 2H), 3.50 (s, 3H), 1.85 ( d, J = 6.8 Hz, 3H). |
| 121 | 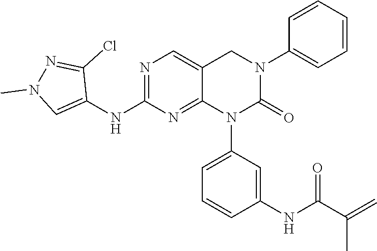 [SMILES: C=C(C)C(=O)Nc1cccc(N2C(=O)N(c3ccccc3)Cc3cnc(Nc4cn(C)nc4Cl)nc32)c1] | X | 515.28 | δ 9.95 (bs, 1H), 8.74 (bs, 1H), 8.21 (s, 1H), 7.80 (d, J = 8.4 Hz, 1H), 7.73 (s, 1H), 7.41-7.51 (m, 5H), 7.25-7.29 (m, 1H), 7.09 (d, J = 7.6 Hz, 1H), 6.85 (s, 1H), 5.81 (s, 1H), 5.52 (s, 1H) 4.88 (s, 2H), 3.52 (s, 3H), 1.94 (s, 3H). |
| 122 | 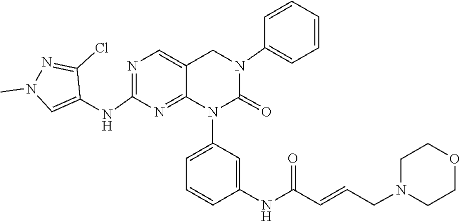 [SMILES: Cn1cc(Nc2ncc3c(n2)N(c2cccc(NC(=O)/C=C/CN4CCOCC4)c2)C(=O)N(c2ccccc2)C3)c(Cl)n1] | X | 600.0 | δ 10.21 (s, 1H), 8.60-8.80 (bs, 2H), 8.19 (s, 1H), 7.90-7.80 (bs, 1H), 7.70 (s, 2H), 7.42 (s, 3H), 7.26 (s, 1H), 7.08 (s, 1H), 6.80-6.60 (m, 2H), 6.40-6.20 (m, 1H), 4.86 (s, 2H), 3.57 (s, 3H), 3.49 (s, 2H), 3.11 (s, 4H), 2.37 (s, 4H). |
| 123 | 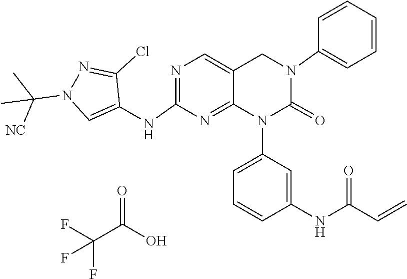 [SMILES: C=CC(=O)Nc1cccc(N2C(=O)N(c3ccccc3)Cc3cnc(Nc4cn(C(C)(C)C#N)nc4Cl)nc32)c1 ; O=C(O)C(F)(F)F] | Y | 554.18 | δ 10.28 (bs, 1H), 8.80 (bs, 1H), 8.22 (s, 1H), 7.62-7.64 (m, 2H), 7.40-7.43 (m, 6H), 7.23-7.21 (m, 2H), 7.06 (d, J = 7.6 Hz, 1H), 6.39-6.46 (m, 1H), 6.22-6.27 (m, 1H), 5.77 (dd, J = 1.6 Hz & 10.0 Hz, 1H), 4.87 (s, 2H), 1.75 (s, 6H). |
| 124 | 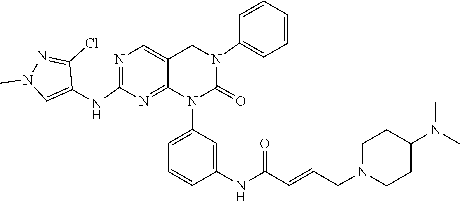 | X | 641.0 | δ 10.19 (s, 1H), 8.75 (bs, 1H), 8.19 (d, J = 8.0 Hz, 2H), 7.71 (d, J = 8.0 Hz, 2H), 7.49-7.41 (m, 4H), 7.25 (s, 1H), 7.06 (d, J = 8.0 Hz, 1H), 6.71 (d, J = 16.0 Hz, 2H), 6.23 (d, J = 16.0 Hz, 1H), 4.86 (s, 2H), 3.48 (s, 3H), 3.06 (d, J = 4.0 Hz, 2H), 2.82 (d, J = 12.0 Hz, 2H), 2.14 (s, 6H), 1.90 (m, 5H), 1.67-1.68 (m, 2H). |
| 125 | 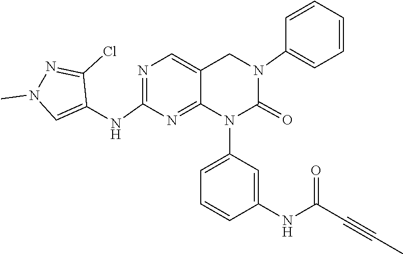 [SMILES: CC#CC(=O)Nc1cccc(N2C(=O)N(c3ccccc3)Cc3cnc(Nc4cn(C)nc4Cl)nc32)c1] | X | 513.1 | 10.77 (s, 1H), 8.18 (s, 1H), 7.63 (s, 2H), 7.48-7.38 (m, 4H), 7.27-7.25 (m, 1H), 7.08 (d, J = 8.0 Hz, 2H), 6.8-6.6 (m, 2H), 4.85 (s, 2H), 3.5-3.83 (m, 3H), 2.02 (s, 3H). |
| 126 | 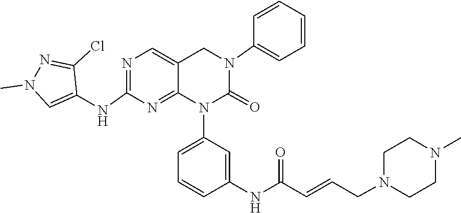 | X | 613.2 | δ 10.23 (s, 1H), 8.9-8.7 (bs, 1H), 8.18 (s, 1H), 7.7 (s, 2H), 7.45-7.41 (m, 4H), 7.25 (s, 1H), 7.07 (s, 1H), 6.70 (d, J = 14.0 Hz, 3H), 6.23 (d, J = 15.2 Hz, 1H), 4.53 (s, 1H), 3.46 (s, 3H), 3.38 (s, 2H), 3.07 (s, 2H), 2.30 (m, 4H), 2.12 (s, 3H), 1.91 (s, 1H), 1.69 (s, 1H), 1.20 (s, 1H). |
| 127 | 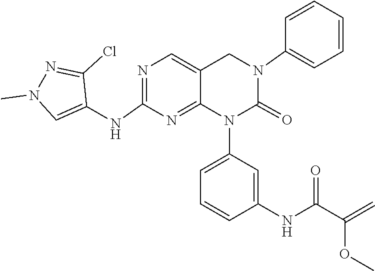 [SMILES: C=C(OC)C(=O)Nc1cccc(N2C(=O)N(c3ccccc3)Cc3cnc(Nc4cn(C)nc4Cl)nc32)c1] | X | 531.13 | δ 10.02 (bs, 1H), 8.81 (bs, 1H), 8.21 (s, 1H), 7.87 (d, J = 7.6 Hz, 1H), 7.81 (s, 1H), 7.41-7.50 (m, 5H), 7.26-7.29 (m, 1H), 7.11 (d, J = 8.0 Hz, 1H), 6.53-6.67 (m, 1H), 5.20 (d, J = 2.0 Hz, 1H), 6.86 (s, 2H), 4.64 (d, J = 1.6 Hz, 1H), 3.69 (s, 3H), 3.50 (s, 3H). |
| 128 | 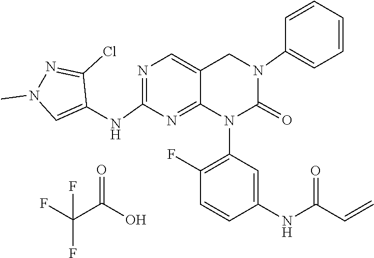 [SMILES: C=CC(=O)Nc1ccc(F)c(N2C(=O)N(c3ccccc3)Cc3cnc(Nc4cn(C)nc4Cl)nc32)c1 ; O=C(O)C(F)(F)F] | Y | 519.3 | δ 10.34 (s, 1 H), 8.82 (bs, 1H), 8.21 (s, 1H), 7.85 (d, J = 4.8 Hz, 1H), 7.74 (d, J = 8.4 Hz, 1H), 7.37-7.43 (m, 5H), 7.26-7.29 (m, 1H), 6.85-7.19 (m, 2H), 6.37-6.43 (m, 1H), 6.22-6.27 (m, 1H), 5.75 (d, J = 5.2 Hz, 1H), 4.48-4.95 (m, 2H), 3.53 (s, 3H). |
| 129 | 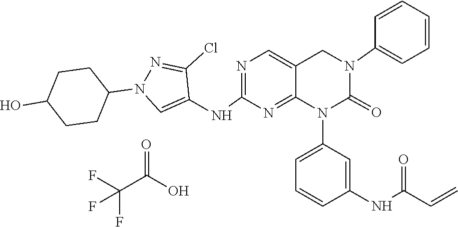 | Y | 585.28 | δ 10.30 (bs, 1H), 8.78 (bs, 1H), 8.22 (s, 1H), 7.72-7.74 (m, 2H), 7.40-7.50 (m, 6H), 7.20-7.27 (m, 1H), 7.09-7.11 (m, 1H), 6.87-6.95 (m, 1H), 6.40-6.47 (m, 1H), 6.24 (d, J = 15.2 Hz, 1H), 5.76 (d, J = 8.4 Hz, 1H), 4.88 (s, 2H), 3.45-3.46 (m, 2H), 1.86-1.88 (m, 2H), 1.56-1.62 (m, 4H), 1.33-1.38 (m 2H). |
| 130 | 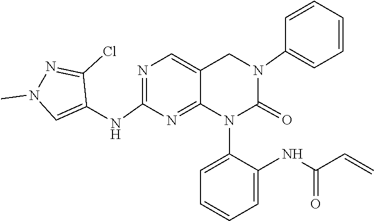 [SMILES: C=CC(=O)Nc1ccccc1N1C(=O)N(c2ccccc2)Cc2cnc(Nc3cn(C)nc3Cl)nc21] | Y | 501.0 | δ 9.61 (s, 1H), 8.69 (bs, 1H), 8.17 (s, 2H), 7.35-7.47 (m, 6H), 7.25 (t, J = 7.6 Hz, 2H), 6.75 (bs, 1H), 6.49-6.56 (m, 1H), 6.16 (d, J = 16.8 Hz, 1H), 5.66 (d, J = 10.0 Hz, 1H). 4.77-4.93 (m, 2H), 3.52 (s, 3H). |
| 131 | 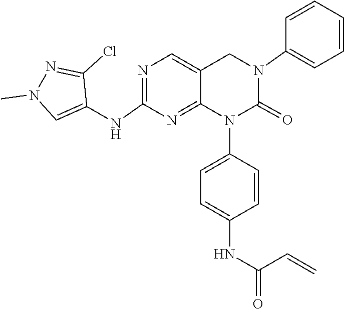 [SMILES: C=CC(=O)Nc1ccc(N2C(=O)N(c3ccccc3)Cc3cnc(Nc4cn(C)nc4Cl)nc32)cc1] | Z | 501.0 | δ 10.33 (s, 1H), 8.73 (bs, 2H), 8.17 (bs, 1H), 6.52 (s, 1H), 7.81 (d, J = 8.0 Hz, 2H), 7.42-7.25 (m, 6H), 6.48-6.42 (m, 1H), 6.30-6.26 (m, 1H), 5.78-5.75 (d, J = 12.0 Hz, 1H,), 4.85 (s, 2H), 3.45 (s, 3H). |

### Scheme 31: Preparation of N-(3-(7-((3-chloro-1-(piperidin-4-yl)-1H-pyrazol-4-yl)amino)-2-oxo-3-phenyl-3,4-dihydropyrimido[4,5-d]pyrimidin-1(2H)-yl)phenyl)acrylamide (Compound 132)

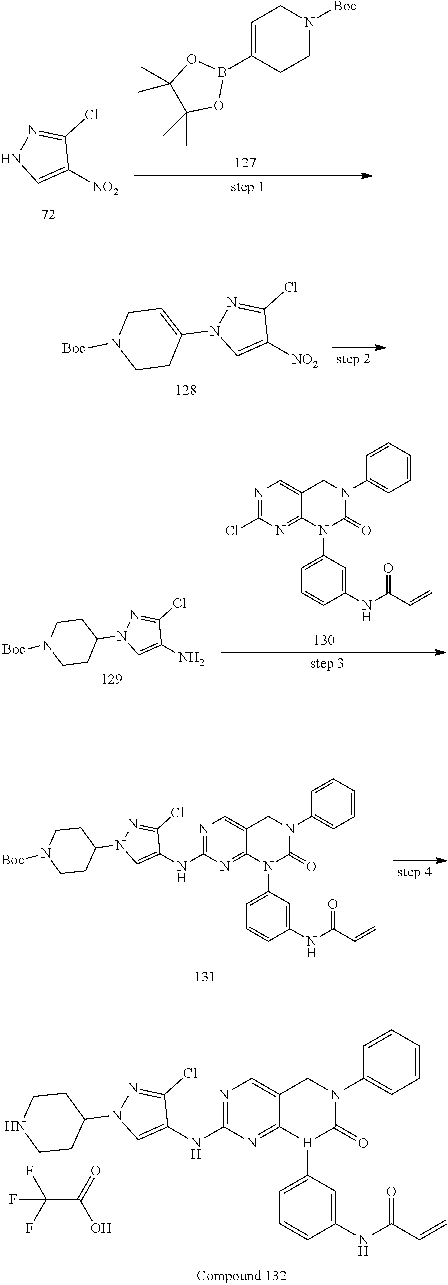

### Step 1: Preparation of tert-butyl 4-(3-chloro-4-nitro-1H-pyrazol-1-yl)-3,6-dihydropyridine-1(2H)-carboxylate (128)

To a stirred solution of 3-chloro-4-nitro-1H-pyrazole (72) (500 mg, 3.39 mmol) in methanol (15.0 mL) were added tert-butyl 4-(4,4,5,5-tetramethyl-1,3,2-dioxaborolan-2-yl)-1,2,3,6-tetrahydropyridine-1-carboxylate (127) (1.26 g, 4.07 mmol) and pyridine (0.54 mL, 6.78 mmol), and the resulting reaction mixture was stirred for 10 minutes. Copper(2+) diacetate (0.92 g, 5.08 mmol) was added and the reaction mixture was stirred at room temperature for 48 hours. The reaction mixture was filtered through celite bed and the solvent was concentrated under reduced pressure. Crude compound was purified by column chromatography, eluted with 30% ethyl acetate in heptane to afford the title compound (128). ¹H NMR (400 MHz, CDCl₃) δ=8.32 (s, 1H), 6.30 (s, 1H), 4.14 (s, 2H), 3.71 (s, 2H), 2.63 (s, 2H), 1.48 (s, 9H).

### Step 2: Preparation of tert-butyl 4-(3-chloro-4-nitro-1H-pyrazol-1-yl)piperidine-1-carboxylate (129)

To a stirred solution of tert-butyl 4-(3-chloro-4-nitro-1H-pyrazol-1-yl)-1,2,3,6-tetrahydropyridine-1-carboxylate (128) (0.25 g, 0.76 mmol) in ethyl acetate (10.0 mL) was added platinum oxide (0.017 g, 0.076 mmol) and applied hydrogen gas (using bladder), stirred at room temperature for 5 hours. The reaction was monitored by TLC and LCMS. After completion of the reaction, the reaction mixture was filtered through celite bed and washed with ethyl acetate. The filtrate was concentrated afforded the crude product and it was purified by flash column chromatography (Combiflash) using a silica gel column and the product was eluted at 2% methanol in dichloromethane. Desired fractions were concentrated under reduced pressure afforded tert-butyl 4-(4-amino-3-chloro-1H-pyrazol-1-yl)piperidine-1-carboxylate (129) (0.2 g, 87% yield) as brown solid. LCMS [M+H]⁺ 245.1

### Step 3: Preparation of tert-butyl 4-[3-chloro-4-({7-oxo-6-phenyl-8-[3-(prop-2-enamido)phenyl]-5H,6H,7H,8H-pyrimido[4,5-d][1,3]diazin-2-yl}amino)-1H-pyrazol-1-yl]piperidine-1-carboxylate (131)

To a stirred solution of tert-butyl 4-(4-amino-3-chloro-1H-pyrazol-1-yl)piperidine-1-carboxylate (129) (70.0 mg, 0.233 mmol) in propan-2-ol (10.0 mL) were added N-(3-{7-chloro-2-oxo-3-phenyl-1H,2H,3H,4H-pyrimido[4,5-d][1,3]diazin-1-yl}phenyl)prop-2-enamide (130) (94.4 mg, 0.233 mmol) and trifluoroacetic acid (0.017 mL, 0.233 mmol). Then the reaction mixture was heated in a sealed tube at 100° C. for 4 hours. After completion of the reaction, the reaction mixture was concentrated under reduced pressure. Then the obtained residue was dissolved in dichloromethane (10 mL) and washed with saturated sodium bicarbonate solution (5 mL), dried over sodium sulfate, filtered and concentrated. It was purified by combiflash chromatography, eluted with 2% methanol in dichloromethane to afford tert-butyl 4-[3-chloro-4-({7-oxo-6-phenyl-8-[3-(prop-2-enamido)phenyl]-5H,6H,7H,8H-pyrimido[4,5-d][1,3]diazin-2-yl}amino)-1H-pyrazol-1-yl]piperidine-1-carboxylate (131) (0.12 g, crude) as semi solid. LCMS [M+H]⁺ 670.3

### Step 4: Preparation of N-[3-(7-{[3-chloro-1-(piperidin-4-yl)-1H-pyrazol-4-yl]amino}-2-oxo-3-phenyl-1H,2H,3H,4H-pyrimido[4,5-d][1,3]diazin-1-yl)phenyl]prop-2-enamide TFA salt (Compound 132)

To a stirred solution of tert-butyl 4-[3-chloro-4-({7-oxo-6-phenyl-8-[3-(prop-2-enamido)phenyl]-5H,6H,7H,8H-pyrimido[4,5-d][1,3]diazin-2-yl}amino)-1H-pyrazol-1-yl]piperidine-1-carboxylate (131) (100 mg, 0.149 mmol) in dichloromethane (5 mL) at 0° C., trifluoroacetic acid (2 mL) was added dropwise and the reaction mixture was stirred at room temperature for 2 hours. After completion of the reaction, the solvent was evaporated under reduced pressure. The crude product was purified by preparative HPLC to give N-[3-(7-{[3-chloro-1-(piperidin-4-yl)-1H-pyrazol-4-yl]amino}-2-oxo-3-phenyl-1H,2H,3H,4H-pyrimido[4,5-d][1,3]diazin-1-yl)phenyl]prop-2-enamide TFA salt (Compound 132) as white solid (20.0 mg, 24%). ¹H NMR (400 MHz, DMSO d₆) δ 10.37 (s, 1H), 8.90 (s, 1H), 8.60 (s, 1H), 8.20 (s, 2H), 7.99 (s, 2H), 7.53-7.49 (m, 2H), 7.42 (s, 2H), 7.26 (s, 1H), 7.11 (d, J=7.6 Hz, 1H), 6.76 (s, 1H), 6.45-6.41 (m, 1H), 6.28-6.23 (m, 1H), 5.78 (d, J=10 Hz, 1H), 4.87 (s, 2H), 3.82 (s, 1H), 3.40-3.37 (m, 3H), 3.20-3.07 (m, 2H), 1.96-1.94 (m, 2H), 1.83-1.75 (m, 2H). LCMS [M+H]⁺ 570.1.

### Scheme 32: Preparation of (R,E)-4-(dimethylamino)-N-(3-(7-((1-methyl-1H-pyrazol-4-yl)amino)-2-oxo-3-(1-phenylethyl)-3,4-dihydropyrimido[4,5-d]pyrimidin-1(2H)-yl)phenyl)but-2-enamide (Compound 133) and (S,E)-4-(dimethylamino)-N-(3-(7-((1-methyl-1H-pyrazol-4-yl)amino)-2-oxo-3-(1-phenylethyl)-3,4-dihydropyrimido[4,5-d]pyrimidin-1(2H)-yl)phenyl)but-2-enamide (Compound 134)

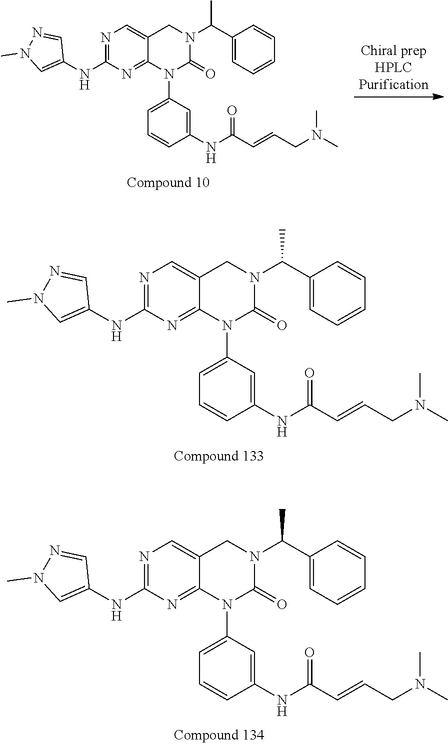

Compound 10 was separated by chiral preparative HPLC purification to afford the title compounds.

Compound 133 (Isomer 1): ¹H NMR (400 MHz, DMSO d₆) δ 10.25 (bs, 1H), 9.36 (bs, 1H), 8.01 (s, 1H), 7.73 (bs, 1H), 7.64 (s, 1H), 7.48 (bs, 1H), 7.37-7.36 (m, 4H), 7.29-7.28 (m, 1H), 7.02-7.01 (m, 2H), 6.74-6.67 (m, 1H), 6.62 (bs, 1H), 6.25-6.21 (m, 1H), 5.70-5.69 (m, 1H), 4.41 (d, J=14.0 Hz, 1H), 3.91 (d, J=14.0 Hz, 1H), 3.47 (s, 3H), 3.04 (d, J=5.6 Hz, 2H), 2.14 (s, 6H), 1.57 (d, J=6.8 Hz, 3H). LCMS [M+H]⁺ 552.3.

Compound 134 (Isomer 2): ¹H NMR (400 MHz, DMSO d₆) δ 10.25 (bs, 1H), 9.36 (bs, 1H), 7.99 (s, 1H), 7.71 (bs, 1H), 7.63 (s, 1H), 7.48 (bs, 1H), 7.35-7.27 (m, 5H), 7.01-6.99 (m, 2H), 6.72-6.67 (m, 2H), 6.21 (d, J=15.6 Hz, 1H), 5.68-5.66 (m, 1H), 4.41 (d, J=14.0 Hz, 1H), 3.91 (d, J=14.0 Hz, 1H), 3.44 (s, 3H), 3.02-3.00 (m, 2H), 2.11 (s, 6H), 1.56 (d, J=6.8 Hz, 3H). LCMS [M+H]⁺ 552.3.

### Scheme 33: Preparation of (E)-4-fluoro-N-(3-(7-((2-fluoropyridin-3-yl)amino)-2-oxo-3-phenyl-3,4-dihydropyrimido[4,5-d]pyrimidin-1(2H)-yl)phenyl)but-2-enamide (Compound 135)

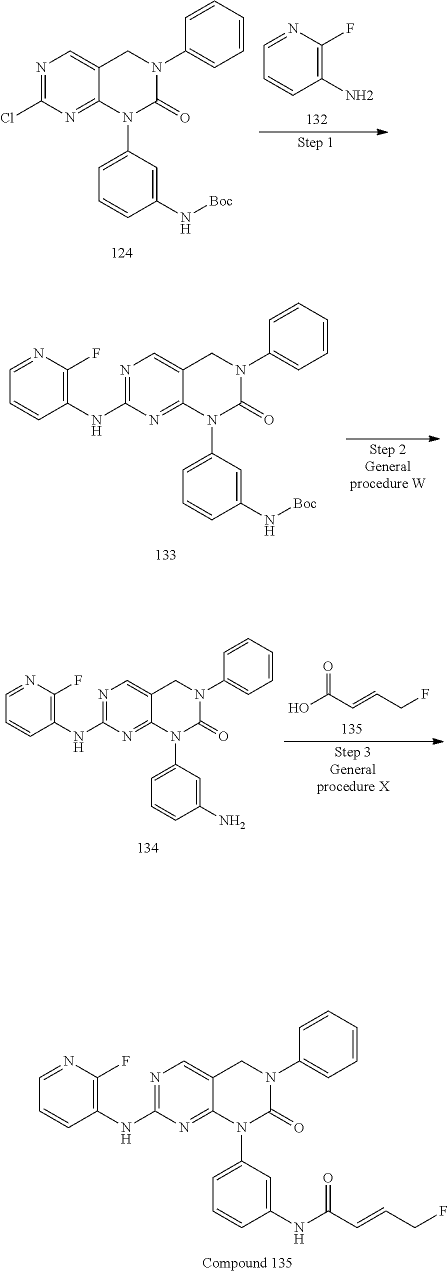

### Step 1: Preparation of tert-butyl (3-(7-((2-fluoropyridin-3-yl)amino)-2-oxo-3-phenyl-3,4-dihydropyrimido[4,5-d]pyrimidin-1(2H)-yl)phenyl)carbamate (133)

To a solution of tert-butyl (3-(7-((2-fluoropyridin-3-yl)amino)-2-oxo-3-phenyl-3,4-dihydropyrimido[4,5-d]pyrimidin-1(2H)-yl)phenyl)carbamate (124) (0.2 g, 0.443 mmol) and 2-fluoropyridin-3-amine (132) (0.059 g, 0.531 mmol) in 1,4-dioxane (3.00 mL) were added potassium carbonate (0.183 g, 1.33 mmol), [2′,6′-bis(propan-2-yloxy)-[1,1′-biphenyl]-2-yl]dicyclohexylphosphane (0.041 g, 0.088 mmol) and the reaction mixture was purged with nitrogen for 10 minutes. To this reaction mixture was added tris(dibenzylideneacetone)dipalladium(0) (0.04 g, 0.044 mmol) and the reaction mixture was heated in microwave at 130° C. for 2 hours. The progress of the reaction was monitored by LCMS. The reaction mixture was diluted with water (20 mL) and extracted with ethyl acetate (2×50 mL). The combined organic layer was washed with brine (5 mL), dried over anhydrous sodium sulfate, filtered and evaporated under reduced pressure. The crude product was purified by combiflash purifier using 50% ethyl acetate in hexane to afford tert-butyl (3-(7-((2-fluoropyridin-3-yl)amino)-2-oxo-3-phenyl-3,4-dihydropyrimido[4,5-d]pyrimidin-1(2H)-yl)phenyl)carbamate (133) (0.15 g) as yellow solid. LCMS: [M+H]⁺ 528

### Step2: Preparation of 1-(3-aminophenyl)-7-((2-fluoropyridin-3-yl)amino)-3-phenyl-3,4-dihydropyrimido[4,5-d]pyrimidin-2(1H)-one (134)

Title compound was prepared in a manner substantially similar to procedure mentioned in General procedure W, as a brown solid (0.13 g, crude) which was used directly for the next step. LCMS: [M+H]⁺ 428.

### Step3: Preparation of (E)-4-fluoro-N-(3-(7-((2-fluoropyridin-3-yl)amino)-2-oxo-3-phenyl-3,4-dihydropyrimido[4,5-d]pyrimidin-1(2H)-yl)phenyl)but-2-enamide (Compound 135)

Title compound was prepared in a manner substantially similar to procedure mentioned in General procedure X, as off white solid (0.005 g, 4%). ¹H NMR (400 MHz, DMSO-d₆): δ10.31 (s, 1H), 9.06 (s, 1H), 8.26 (s, 1H), 7.87 (t, J=10.6 Hz, 1H), 7.74 (s, 1H), 7.68-7.65 (m, 2H), 7.44 (s, 5H), 7.28 (bs, 1H), 7.08 (d, J=7.6 Hz, 1H), 6.91-6.81 (m, 2H), 6.36 (d, J=15.60 Hz, 1H), 5.17 (d, J=46.4 Hz, 2H), 4.91 (s, 2H). LCMS: [M+H]⁺ 514.

### Scheme 34: Preparation of N-(3-(7-((3-chloro-1-(oxetan-3-yl)-1H-pyrazol-4-yl)amino)-2-oxo-3-phenyl-3,4-dihydropyrimido[4,5-d]pyrimidin-1(2H)-yl)phenyl)acrylamide (Compound 136)

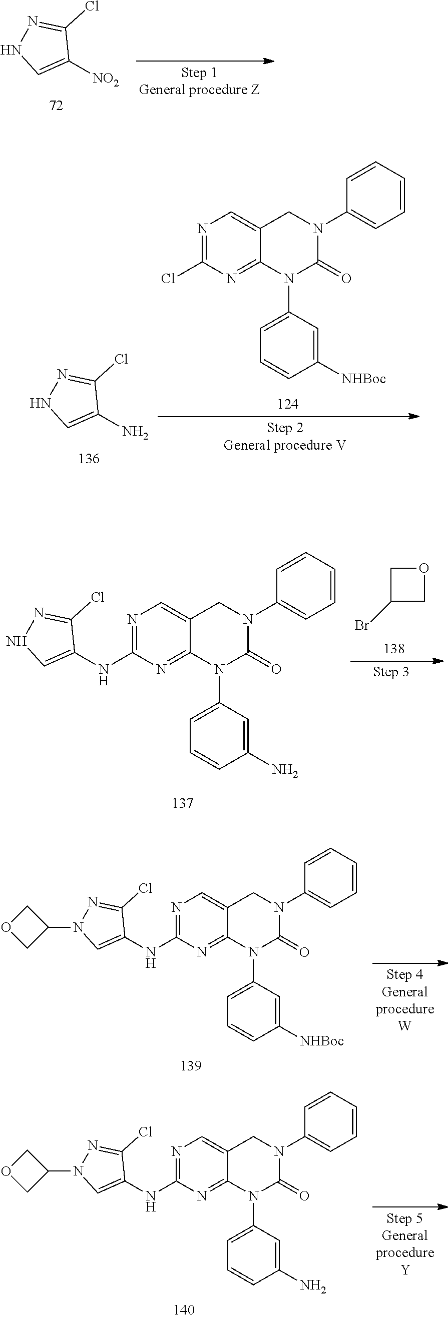

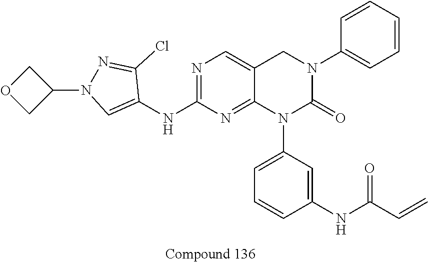 [SMILES: C=CC(=O)Nc1cccc(N2C(=O)N(c3ccccc3)Cc3cnc(Nc4cn(C5COC5)nc4Cl)nc32)c1]

### Step 1: Preparation of 3-chloro-1H-pyrazol-4-amine (136)

Title compound was prepared in a manner substantially similar to procedure mentioned in General procedure Z. LCMS: [M+H]⁺ 117.78.

### Step 2: Preparation of tert-butyl (3-(7-((3-chloro-1H-pyrazol-4-yl)amino)-2-oxo-3-phenyl-3,4-dihydropyrimido[4,5-d]pyrimidin-1(2H)-yl)phenyl)carbamate (137)

Title compound was prepared in a manner substantially similar to procedure mentioned in General procedure V, as yellow solid (0.6 g, 51%). LCMS: [M+H]⁺ 533.41.

### Step 3: Preparation of tert-butyl (3-(7-((3-chloro-1-(oxetan-3-yl)-1H-pyrazol-4-yl)amino)-2-oxo-3-phenyl-3,4-dihydropyrimido[4,5-d]pyrimidin-1(2H)-yl)phenyl)carbamate (139)

To stirred a solution of tert-butyl (3-(7-((3-chloro-1H-pyrazol-4-yl)amino)-2-oxo-3-phenyl-3,4-dihydropyrimido[4,5-d]pyrimidin-1(2H)-yl)phenyl)carbamate (137) (230 mg, 0.432 mmol) in N,N-dimethylformamide (5.00 mL) was added cesium carbonate (422 mg, 1.29 mmol), and the reaction mixture was stirred at room temperature for 5 minutes. 3-bromooxetane (138) (70.9 mg, 0.518 mmol) was added and the reaction mixture was heated at 140° C. for 2 hours in microwave. The progress of the reaction was monitored by TLC and LCMS analysis. After completion of the reaction, the reaction mixture was poured into ice cold water (25 mL) and extracted with ethyl acetate (25 mL×2). The combined organic layers were dried over sodium sulfate and concentrated under reduced pressure. The crude product was purified by combiflash purifier and was eluted with 65% ethyl acetate in hexane to afford the title compound as off white solid (0.09 g, 28%). LCMS: [M+H]⁺ 489.39 (deboc mass).

### Step 4: Preparation of 1-(3-aminophenyl)-7-((3-chloro-1-(oxetan-3-yl)-1H-pyrazol-4-yl)amino)-3-phenyl-3,4-dihydropyrimido[4,5-d]pyrimidin-2(1H)-one (140)

Title compound was prepared in a manner substantially similar to procedure mentioned in General procedure W. LCMS: [M+H]⁺ 489.39.

### Step 5: Preparation of N-(3-(7-((3-chloro-1-(oxetan-3-yl)-1H-pyrazol-4-yl)amino)-2-oxo-3-phenyl-3,4-dihydropyrimido[4,5-d]pyrimidin-1(2H)-yl)phenyl)acrylamide (Compound 136)

Title compound was prepared in a manner substantially similar to procedure mentioned in General procedure Y as off white solid (20 mg, 19%). ¹H NMR (400 MHz, DMSO-d₆): δ 10.36 (s, 1H), 8.89 (bs, 1H), 8.22 (s, 1H), 7.80-7.78 (m, 1H), 7.71 (bs, 1H), 7.52-7.48 (m, 1H), 7.44-7.40 (m, 4H), 7.29-7.26 (m, 1H), 7.10 (d, J=7.6 Hz, 1H), 6.85 (bs, 1H), 6.48-6.41 (m, 1H), 6.29-6.24 (m, 1H), 5.79-5.76 (m, 1H), 5.00 (bs, 1H), 4.88 (s, 2H), 4.81 (t, J=7.2 Hz, 2H), 4.69 (bs, 2H). LCMS: [M+H]⁺ 543.08.

### Example 2: EGFR L858R & Del19/T790M Assay Protocol (to Find the IC₅₀ for the Compounds Using HTRF Method)

### Reagents for Enzyme Assay:

1. Enzyme Assay Buffer: 60 mM HEPES (pH 7.4), 50 mM NaCl, 20 mM MgCl₂, 5 mM MnCl₂, filtered using 0.2 μm pore size, stored at 4° C.
   Supplements (added fresh to the enzyme buffer): 1M DTT stock was made, 5% BSA stock and 0.1M Na₃OV₄ stored at 4° C. Enzyme assay buffer 50 mL, 1 M DTT 100 μL, 5% BSA 500 μL, 0.1 M Na₃OV₄ 50 μL.
2. GST-hEGFR (L858R), active: EGFR (T790M/L858R), active (Proqinase), 3.6 μM stock; 2.5 nM in final assay.
3. Gastrin Precusor-Biotinylated: Gastrin precursor (Tyr87) biotinylated peptide, EEAY*GWM, Cell Signaling Tech 1310, Lot-7, Want final 0.5 μM in assay.
4. ATP: 10 mM stock in 100 μL Enzyme assay buffer pH 7.4 prepared freshly, want 5 μM in final assay.
5. Test compounds: Powders were dissolved in 100% DMSO and made up to a final stock concentration of 10 mM (volume in μL to add for 10 mM=mg powder X 10⁵/MW). Final compound concentration in the assay plate started at 10 μM.

### Reagents for HTRF Assay:

1. HTRF buffer: 50 mM Tris —HCl, pH-7.5, 100 mM NaCl, 0.1% BSA, 0.05% Tween20, 0.5 mM EDTA, filtered using 0.2 μm pore size, stored at 4° C.
   Phycolink® Streptavidin-Allophycocyanin (SA-APC) Prozyme, Cat. #PJ25S, Lot #896 085, 2.06 mg/mL, APC concentration is 11.6 μM, streptavidin concentration is 15.5 μM. Want 12 nM final in assay (based on streptavidin concentration).
2. (Europium) Eu-W1024 Anti-phosphotyrosine (PT-66) Antibody: Perkin Elmer (Product Number: AD0068), monoclonal IgG1 that recognizes phosphotyrosine peptide 100 μg/ml stock. Want 0.1 nM final in assay.

### Assay Procedure

1. The compound dilution was made by TECAN as per the above scheme.
2. 16 μL of diluted compound was added to the assay plate by using TECAN.
3. 10 μL of enzyme mix was added to each well except mins.
4. 20 μL of substrate and ATP mix were added into each well of the assay plate, manually.
5. The plate was incubated on a laboratory shaker at room temperature for 60 min.
6. Meanwhile, the HTRF mix was prepared and 75 μL was added to the HTRF plate.
7. When the incubation was over, 10 μL of the reaction mixture was transferred to the HTRF assay plate and incubated on a laboratory shaker for 30 min at room temperature.
8. The reading was taken in Pherastar (ext 337 nm, Em 665 & 620 nm).
9. The results were analyzed using Graphpad prism after calculation of the z factor, signal window and % inhibition to get the IC₅₀.

Table 1 shows the activity of compounds of this invention in the EGFR (L858R & del19/T790M) inhibition assay and (H1975) proliferation assay.

TABLE 1
Biochemical EGFR (L858R & del19/T790M) inhibition data and (H1975)
proliferation data.

| | | | Proliferation_Assay |
|---|---|---|---|
| | | EGFR_L858R | H1975 cells-EC₅₀_nM |
| Cmpd No. | Structure | T790M_IC₅₀_nM | L858R_T790M_mutant |
| 1 | 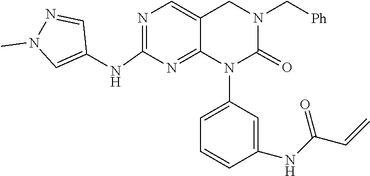 [SMILES: C=CC(=O)Nc1cccc(N2C(=O)N(Cc3ccccc3)Cc3cnc(Nc4cnn(C)c4)nc32)c1] | 0.4 | 6.8 |
| 2 | 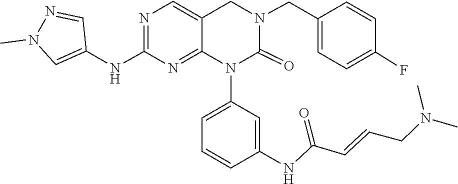 | 2.0 | 19.7 |
| 3 | 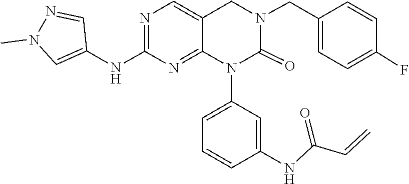 [SMILES: C=CC(=O)Nc1cccc(N2C(=O)N(Cc3ccc(F)cc3)Cc3cnc(Nc4cnn(C)c4)nc32)c1] | 13 | 40.9 |
| 4 | 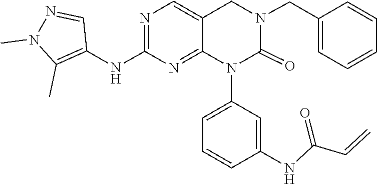 [SMILES: C=CC(=O)Nc1cccc(N2C(=O)N(Cc3ccccc3)Cc3cnc(Nc4cnn(C)c4C)nc32)c1] | 2.4 | 4.9 |
| 5 | 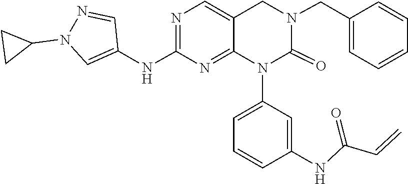 [SMILES: C=CC(=O)Nc1cccc(N2C(=O)N(Cc3ccccc3)Cc3cnc(Nc4cnn(C5CC5)c4)nc32)c1] | 1.0 | 5.7 |
| 6 | 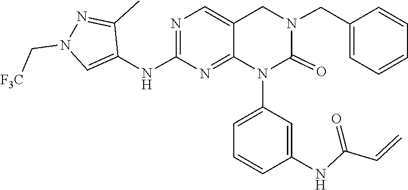 [SMILES: C=CC(=O)Nc1cccc(N2C(=O)N(Cc3ccccc3)Cc3cnc(Nc4cn(CC(F)(F)F)nc4C)nc32)c1] | 1.0 | 8.1 |
| 7 | 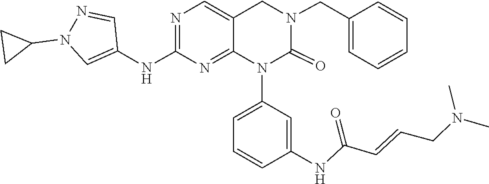 [SMILES: CN(C)C/C=C/C(=O)Nc1cccc(N2C(=O)N(Cc3ccccc3)Cc3cnc(Nc4cnn(C5CC5)c4)nc32)c1] | 6.0 | 9.3 |
| 8 | 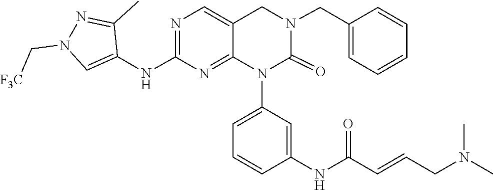 [SMILES: Cc1nn(CC(F)(F)F)cc1Nc1ncc2c(n1)N(c1cccc(NC(=O)/C=C/CN(C)C)c1)C(=O)N(Cc1ccccc1)C2] | 9.0 | 9.2 |
| 9 | 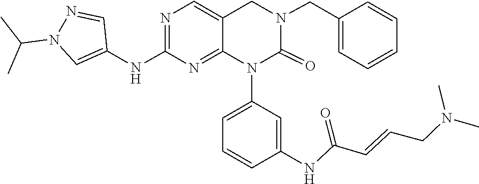 [SMILES: CC(C)n1cc(Nc2ncc3c(n2)N(c2cccc(NC(=O)/C=C/CN(C)C)c2)C(=O)N(Cc2ccccc2)C3)cn1] | 4.0 | 17.6 |
| 10 | 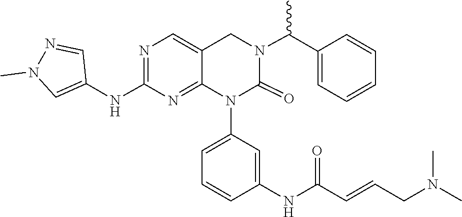 | 4.0 | 4.5 |
| 11 | 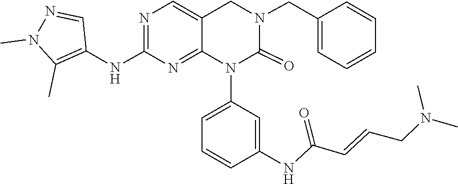 | 2.0 | 9.6 |
| 12 | 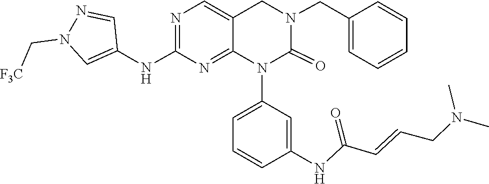 [SMILES: CN(C)C/C=C/C(=O)Nc1cccc(N2C(=O)N(Cc3ccccc3)Cc3cnc(Nc4cnn(CC(F)(F)F)c4)nc32)c1] | 3.0 | 6.6 |
| 13 | 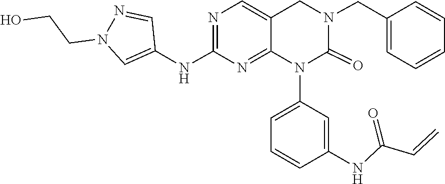 [SMILES: C=CC(=O)Nc1cccc(N2C(=O)N(Cc3ccccc3)Cc3cnc(Nc4cnn(CCO)c4)nc32)c1] | 1.0 | 1.5 |
| 14 | 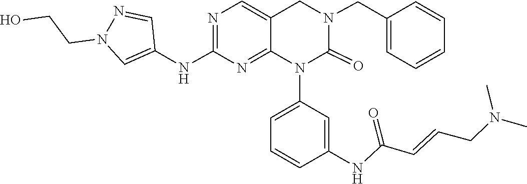 [SMILES: CN(C)C/C=C/C(=O)Nc1cccc(N2C(=O)N(Cc3ccccc3)Cc3cnc(Nc4cnn(CCO)c4)nc32)c1] | 5.0 | 37.8 |
| 15 | 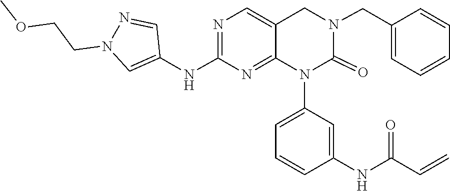 [SMILES: C=CC(=O)Nc1cccc(N2C(=O)N(Cc3ccccc3)Cc3cnc(Nc4cnn(CCOC)c4)nc32)c1] | 2.0 | 1.8 |
| 16 | 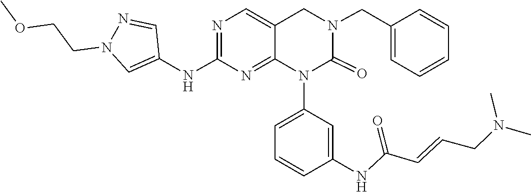 | 1.0 | 15.7 |
| 17 | 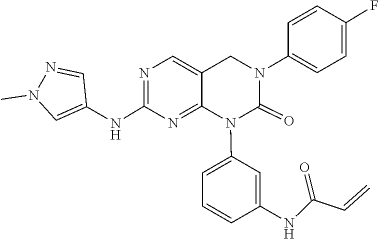 [SMILES: C=CC(=O)Nc1cccc(N2C(=O)N(c3ccc(F)cc3)Cc3cnc(Nc4cnn(C)c4)nc32)c1] | 0.5 | 0.7 |
| 18 | 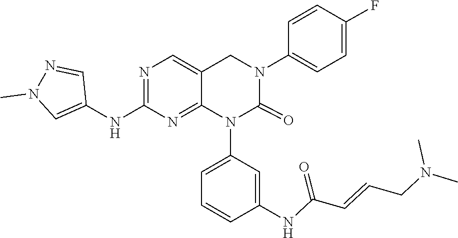 | 2.5 | 34.0 |
| 19 | 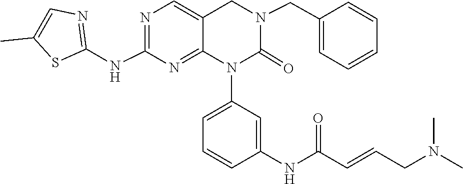 | 6.0 | 16.0 |
| 20 | 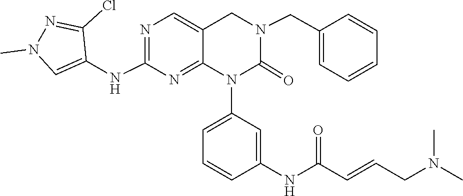 | 1.0 | 2.0 |
| 21 | 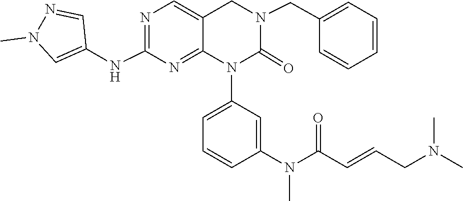 | 45 | 83.9 |
| 22 | 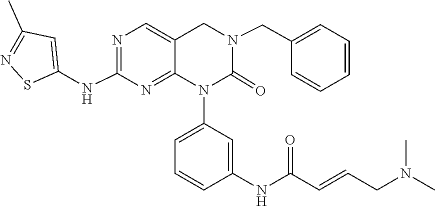 [SMILES: Cc1cc(Nc2ncc3c(n2)N(c2cccc(NC(=O)/C=C/CN(C)C)c2)C(=O)N(Cc2ccccc2)C3)sn1] | 7.0 | 10 |
| 23 | 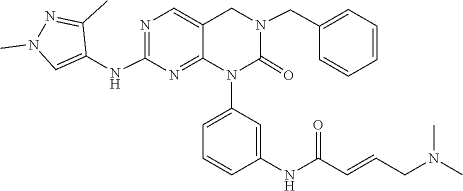 | 3.0 | 6.4 |
| 24 | 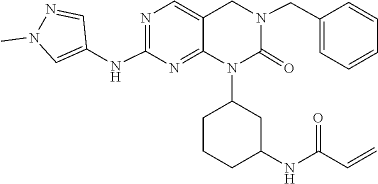 [SMILES: C=CC(=O)NC1CCCC(N2C(=O)N(Cc3ccccc3)Cc3cnc(Nc4cnn(C)c4)nc32)C1] | 53 | 71 |
| 25 | 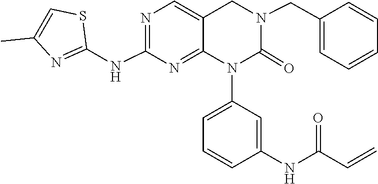 [SMILES: C=CC(=O)Nc1cccc(N2C(=O)N(Cc3ccccc3)Cc3cnc(Nc4nc(C)cs4)nc32)c1] | 3.0 | 29.0 |
| 26 | 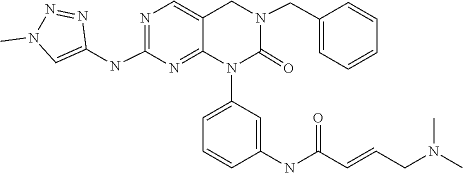 | 5.0 | 221.5 |
| 27 | 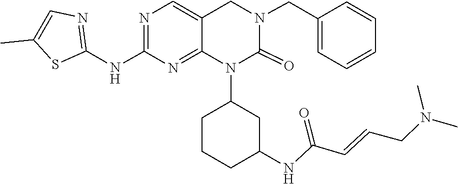 | 303 | 10000 |
| 28 | 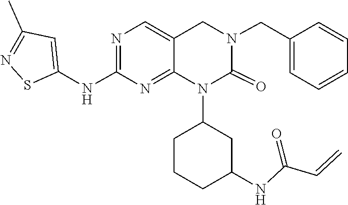 [SMILES: C=CC(=O)NC1CCCC(N2C(=O)N(Cc3ccccc3)Cc3cnc(Nc4cc(C)ns4)nc32)C1] | 28 | 75.4 |
| 29 | 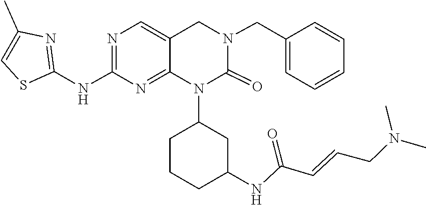 [SMILES: Cc1csc(Nc2ncc3c(n2)N(C2CCCC(NC(=O)/C=C/CN(C)C)C2)C(=O)N(Cc2ccccc2)C3)n1] | 660 | 1079 |
| 30 | 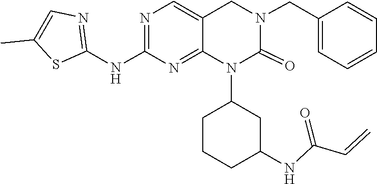 [SMILES: C=CC(=O)NC1CCCC(N2C(=O)N(Cc3ccccc3)Cc3cnc(Nc4ncc(C)s4)nc32)C1] | 164 | 413 |
| 31 | 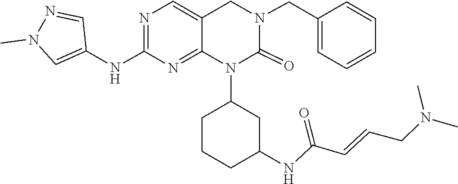 | 281 | 299 |
| 32 | 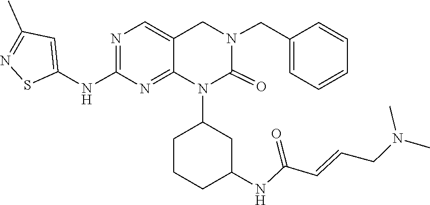 [SMILES: Cc1cc(Nc2ncc3c(n2)N(C2CCCC(NC(=O)/C=C/CN(C)C)C2)C(=O)N(Cc2ccccc2)C3)sn1] | 82 | 176 |
| 33 | 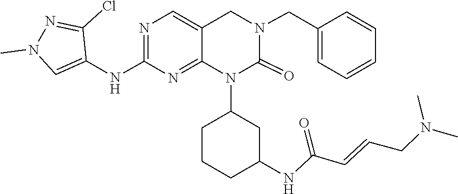 | 869 | ND |
| 34 | 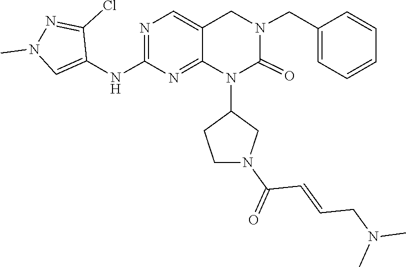 | 6574 | ND |
| 35 | 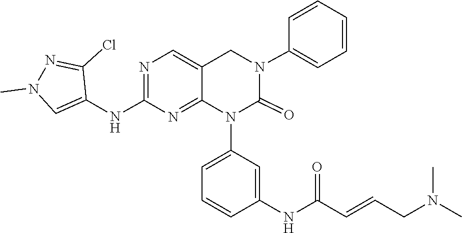 | 7.8 | 79 |
| 36 | 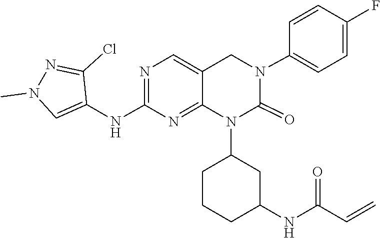 [SMILES: C=CC(=O)NC1CCCC(N2C(=O)N(c3ccc(F)cc3)Cc3cnc(Nc4cn(C)nc4Cl)nc32)C1] | 2.97 | ND |
| 37 (racemic) | 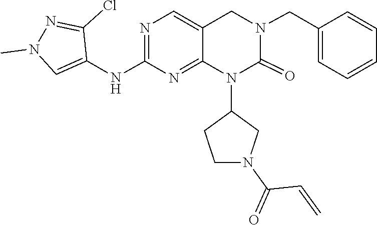 [SMILES: C=CC(=O)N1CCC(N2C(=O)N(Cc3ccccc3)Cc3cnc(Nc4cn(C)nc4Cl)nc32)C1] | 167 | 372 |
| 38 (Enantiomer 1) | 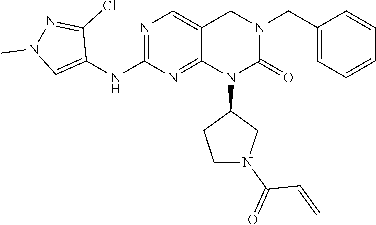 [SMILES: C=CC(=O)N1CC[C@@H](N2C(=O)N(Cc3ccccc3)Cc3cnc(Nc4cn(C)nc4Cl)nc32)C1] | 446 | ND |
| 39 (Enantiomer 2) | 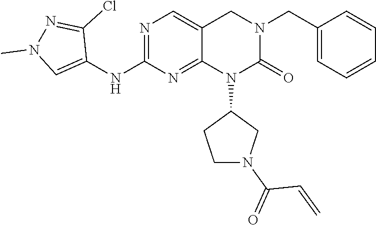 [SMILES: C=CC(=O)N1CC[C@H](N2C(=O)N(Cc3ccccc3)Cc3cnc(Nc4cn(C)nc4Cl)nc32)C1] | 112 | 637 |
| 40 | 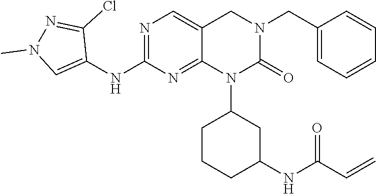 [SMILES: C=CC(=O)NC1CCCC(N2C(=O)N(Cc3ccccc3)Cc3cnc(Nc4cn(C)nc4Cl)nc32)C1] | 98 | 323 |
| 41 | 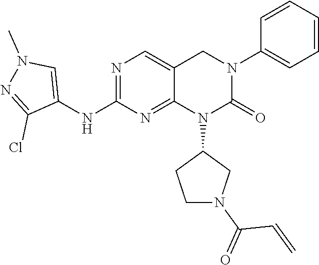 [SMILES: C=CC(=O)N1CC[C@H](N2C(=O)N(c3ccccc3)Cc3cnc(Nc4cn(C)nc4Cl)nc32)C1] | 580 | ND |
| 42 | 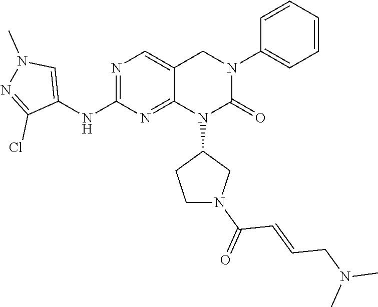 [SMILES: CN(C)C/C=C/C(=O)N1CC[C@H](N2C(=O)N(c3ccccc3)Cc3cnc(Nc4cn(C)nc4Cl)nc32)C1] | 10000 | ND |
| 43 | 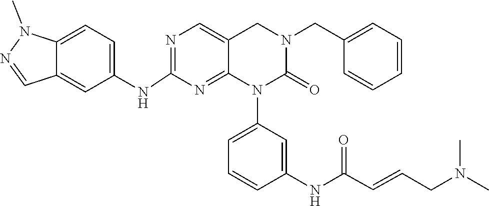 [SMILES: CN(C)C/C=C/C(=O)Nc1cccc(N2C(=O)N(Cc3ccccc3)Cc3cnc(Nc4ccc5c(cnn5C)c4)nc32)c1] | 3.8 | 6.3 |
| 44 | 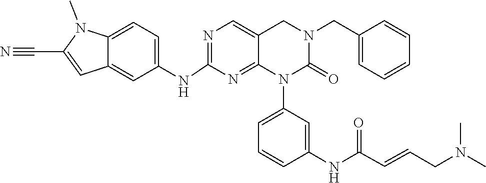 [SMILES: CN(C)C/C=C/C(=O)Nc1cccc(N2C(=O)N(Cc3ccccc3)Cc3cnc(Nc4ccc5c(c4)cc(C#N)n5C)nc32)c1] | 9.0 | 37.6 |
| 45 | 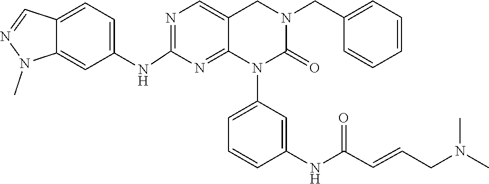 [SMILES: CN(C)C/C=C/C(=O)Nc1cccc(N2C(=O)N(Cc3ccccc3)Cc3cnc(Nc4ccc5cnn(C)c5c4)nc32)c1] | 11.0 | 46.7 |
| 46 | 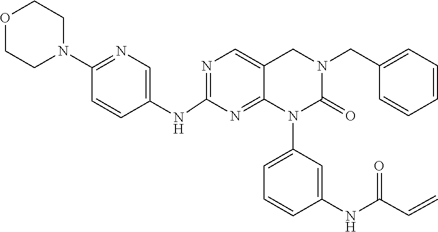 [SMILES: C=CC(=O)Nc1cccc(N2C(=O)N(Cc3ccccc3)Cc3cnc(Nc4ccc(N5CCOCC5)nc4)nc32)c1] | 2.6 | 3.7 |
| 47 | 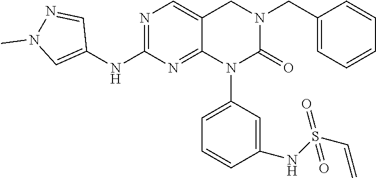 [SMILES: C=CS(=O)(=O)Nc1cccc(N2C(=O)N(Cc3ccccc3)Cc3cnc(Nc4cnn(C)c4)nc32)c1] | 0.9 | 819 |
| 48 | 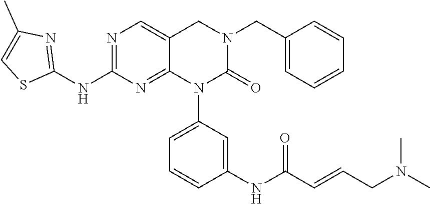 [SMILES: Cc1csc(Nc2ncc3c(n2)N(c2cccc(NC(=O)/C=C/CN(C)C)c2)C(=O)N(Cc2ccccc2)C3)n1] | 3.5 | 133 |
| 49 | 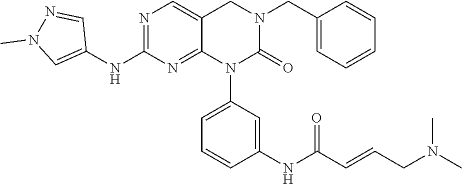 | 0.6 | 2.1 |
| 50 | 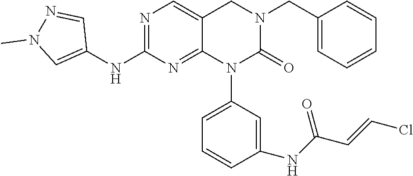 [SMILES: Cn1cc(Nc2ncc3c(n2)N(c2cccc(NC(=O)/C=C/Cl)c2)C(=O)N(Cc2ccccc2)C3)cn1] | 0.4 | 4198 |
| 51 | 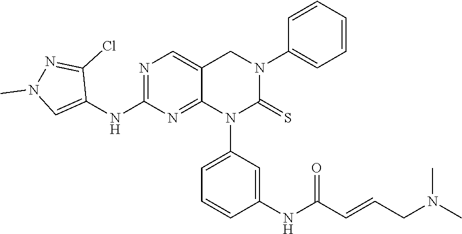 | 8.0 | 9.8 |

ND = Not done

### Example 3: Cellular Proliferation (Alamar Blue) Assays

### Cell Line Details:

1. EGFR(D770_N771insSVD) expressing Ba/F3 stable cell line
2. EGFR (A767_dupASV) expressing Ba/F3 stable cell line
3. A431 cells
4. BTER2 (A775_G776insYVMA) expressing Ba/F3 stable cell line
5. BTER2 (WT) expressing Ba/F3 stable cell line
6. BT-474 cells

### Assay Procedure:

1. Seed cells at 5000 for A431 and 15,000 cells for Ba/F3 in 100 μL/well in complete media (for A431: DMEM with 10% FBS and for Ba/F3 cells: RPMI with 10% FBS) in 96-well tissue culture plate. Leave outer wells without cells for background measurements. Incubate at 37 degree Celsius in 5% CO₂ humidified incubator for 16-18 hours.
2. Add 0.025 ml of 5× concentration compound dilution or DMSO control. Final compound concentration range is 10-0.0005 μM prepared in 3-fold serial dilutions. Incubate for 72 hr at 37 degree Celsius in 500 CO₂ humidified incubator.
3. Add 0.0125 mL Alamar Blue™ reagent to each well with multi-channel pipette and tap gently on each side of the plate to mix. Incubate for 3 hours at 37 degree Celsius in 500 CO₂ humidified incubator.
4. Read plates on fluorescence reader (Tecan Spark Control, Device: Spark, Serial #: 1801006040) at 540 nm excitation, 590 nm emission wavelength.
5. Data analysis was performed using XLfit 5.5.0.5.

Table 2 shows the activity of compounds of this invention in the EGFR cellular proliferation assays.

TABLE 2
Cellular proliferation data.

| Cmpd | | A431 IC₅₀ | A767 IC₅₀ | D770 IC₅₀ |
|---|---|---|---|---|
| No. | Structure | (nM) | (nM) | (nM) |
| 1 | 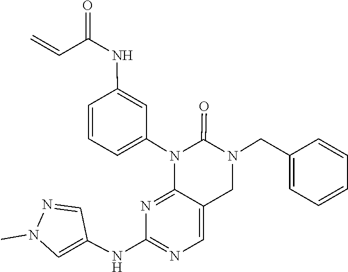 [SMILES: C=CC(=O)Nc1cccc(N2C(=O)N(Cc3ccccc3)Cc3cnc(Nc4cnn(C)c4)nc32)c1] | 65 | 14 | 4 |
| 2 | 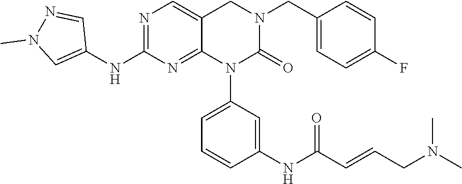 | 615 | 414 | 893 |
| 3 | 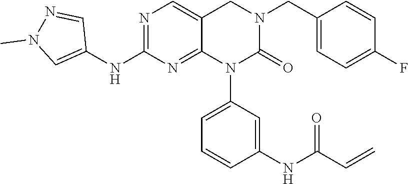 [SMILES: C=CC(=O)Nc1cccc(N2C(=O)N(Cc3ccc(F)cc3)Cc3cnc(Nc4cnn(C)c4)nc32)c1] | 676 | 184 | 131 |
| 4 | 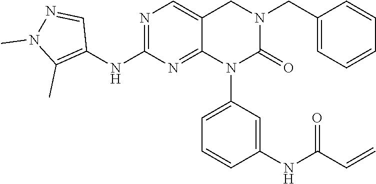 [SMILES: C=CC(=O)Nc1cccc(N2C(=O)N(Cc3ccccc3)Cc3cnc(Nc4cnn(C)c4C)nc32)c1] | 78 | 57 | 40 |
| 5 | 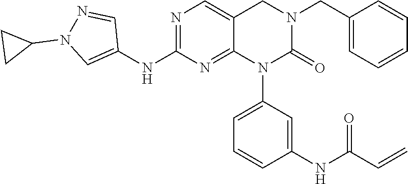 [SMILES: C=CC(=O)Nc1cccc(N2C(=O)N(Cc3ccccc3)Cc3cnc(Nc4cnn(C5CC5)c4)nc32)c1] | ND | ND | ND |
| 6 | 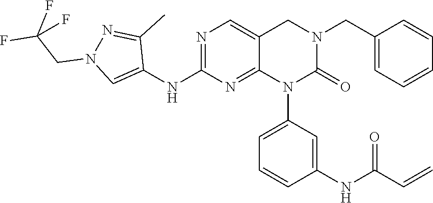 [SMILES: C=CC(=O)Nc1cccc(N2C(=O)N(Cc3ccccc3)Cc3cnc(Nc4cn(CC(F)(F)F)nc4C)nc32)c1] | 1890 | 9 | 9 |
| 7 | 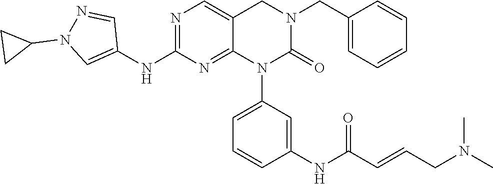 [SMILES: CN(C)C/C=C/C(=O)Nc1cccc(N2C(=O)N(Cc3ccccc3)Cc3cnc(Nc4cnn(C5CC5)c4)nc32)c1] | ND | ND | ND |
| 8 | 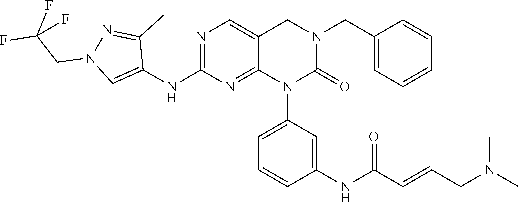 [SMILES: Cc1nn(CC(F)(F)F)cc1Nc1ncc2c(n1)N(c1cccc(NC(=O)/C=C/CN(C)C)c1)C(=O)N(Cc1ccccc1)C2] | 580 | 103 | 108 |
| 9 | 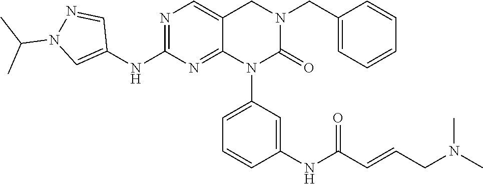 [SMILES: CC(C)n1cc(Nc2ncc3c(n2)N(c2cccc(NC(=O)/C=C/CN(C)C)c2)C(=O)N(Cc2ccccc2)C3)cn1] | 6870 | 103 | 114 |
| 10 | 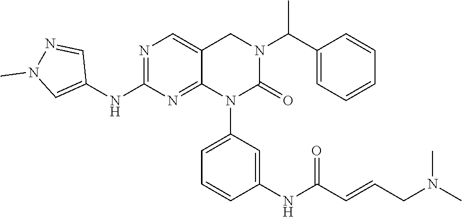 | 638 | 105 | 108 |
| 11 | 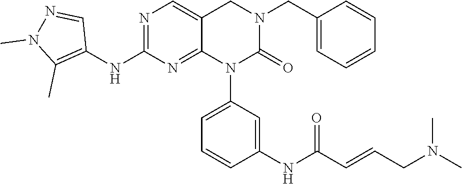 | 1712 | 1154 | 2433 |
| 12 | 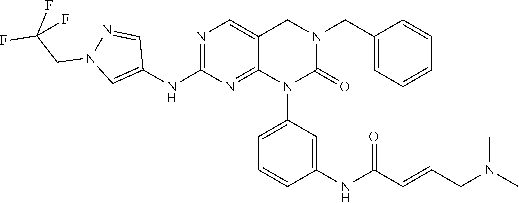 [SMILES: CN(C)C/C=C/C(=O)Nc1cccc(N2C(=O)N(Cc3ccccc3)Cc3cnc(Nc4cnn(CC(F)(F)F)c4)nc32)c1] | 1243 | 106 | 134 |
| 13 | 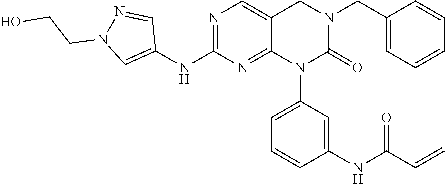 [SMILES: C=CC(=O)Nc1cccc(N2C(=O)N(Cc3ccccc3)Cc3cnc(Nc4cnn(CCO)c4)nc32)c1] | 59 | 45 | 42 |
| 14 | 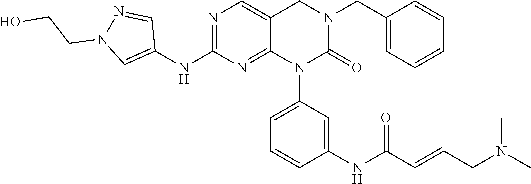 [SMILES: CN(C)C/C=C/C(=O)Nc1cccc(N2C(=O)N(Cc3ccccc3)Cc3cnc(Nc4cnn(CCO)c4)nc32)c1] | 6085 | 2568 | 2920 |
| 15 | 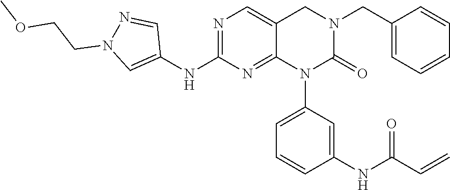 [SMILES: C=CC(=O)Nc1cccc(N2C(=O)N(Cc3ccccc3)Cc3cnc(Nc4cnn(CCOC)c4)nc32)c1] | 77 | 10 | 6 |
| 16 | 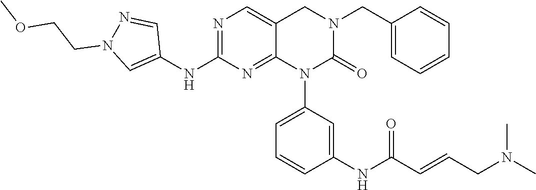 | 371 | 1168 | 2314 |
| 17 | 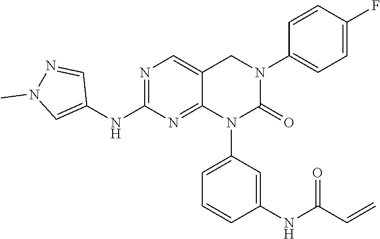 [SMILES: C=CC(=O)Nc1cccc(N2C(=O)N(c3ccc(F)cc3)Cc3cnc(Nc4cnn(C)c4)nc32)c1] | ND | ND | ND |
| 18 | 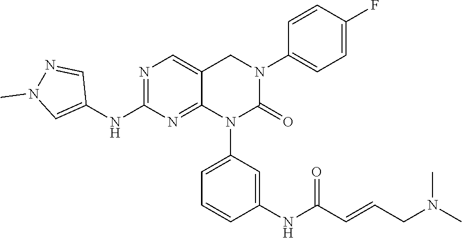 | 332 | 421 | 1102 |
| 19 | 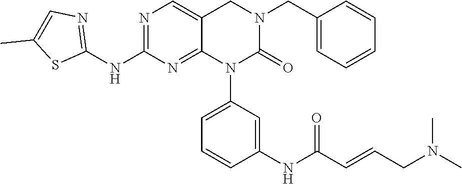 | ND | ND | ND |
| 20 | 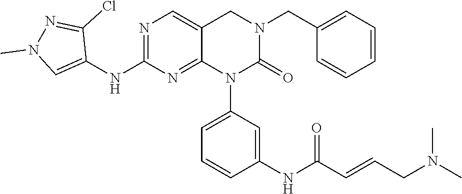 | 595 | 34 | 35 |
| 21 | 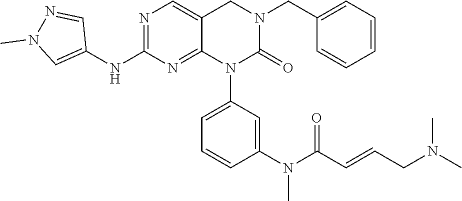 | 2077 | 2814 | 3351 |
| 22 | 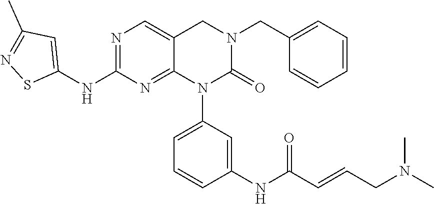 [SMILES: Cc1cc(Nc2ncc3c(n2)N(c2cccc(NC(=O)/C=C/CN(C)C)c2)C(=O)N(Cc2ccccc2)C3)sn1] | 46 | 121 | 278 |
| 23 | 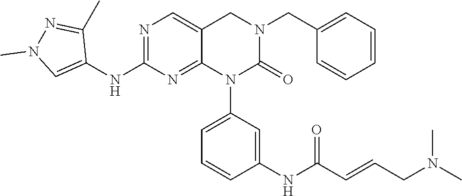 | 18 | 124 | 299 |
| 24 | 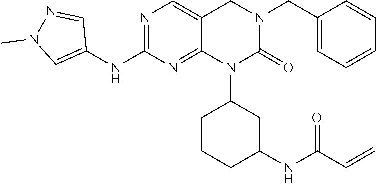 [SMILES: C=CC(=O)NC1CCCC(N2C(=O)N(Cc3ccccc3)Cc3cnc(Nc4cnn(C)c4)nc32)C1] | 8336 | 356 | 340 |
| 25 | 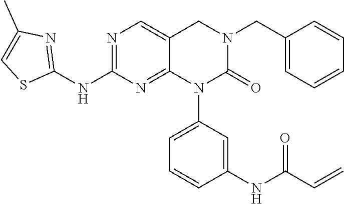 [SMILES: C=CC(=O)Nc1cccc(N2C(=O)N(Cc3ccccc3)Cc3cnc(Nc4nc(C)cs4)nc32)c1] | 83 | 320 | 69 |
| 26 | 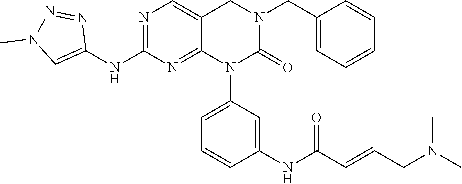 | 3083 | 3326 | 3691 |
| 27 | 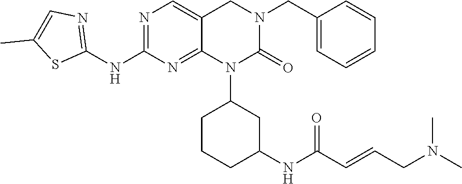 | 4403 | 9192 | 10000 |
| 28 | 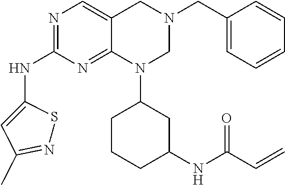 [SMILES: C=CC(=O)NC1CCCC(N2CN(Cc3ccccc3)Cc3cnc(Nc4cc(C)ns4)nc32)C1] | 150 | 417 | 149 |
| 29 | 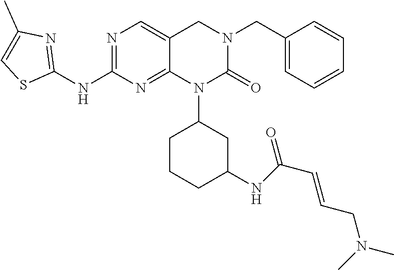 [SMILES: Cc1csc(Nc2ncc3c(n2)N(C2CCCC(NC(=O)/C=C/CN(C)C)C2)C(=O)N(Cc2ccccc2)C3)n1] | 4089 | 2600 | 431 |
| 30 | 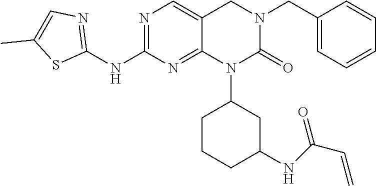 [SMILES: C=CC(=O)NC1CCCC(N2C(=O)N(Cc3ccccc3)Cc3cnc(Nc4ncc(C)s4)nc32)C1] | 920 | 2805 | 2568 |
| 31 | 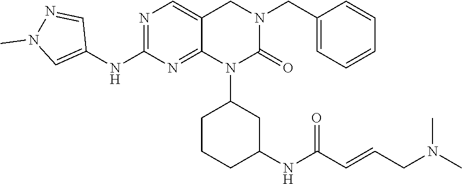 | 1974 | 3319 | 3480 |
| 32 | 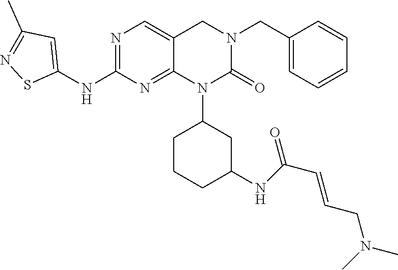 [SMILES: Cc1cc(Nc2ncc3c(n2)N(C2CCCC(NC(=O)/C=C/CN(C)C)C2)C(=O)N(Cc2ccccc2)C3)sn1] | 1373 | 2906 | 2879 |
| 33 | 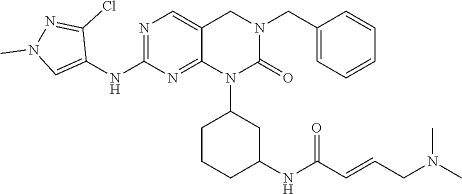 | 10000 | 4077 | 3876 |
| 34 | 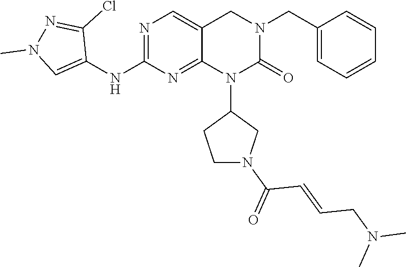 | ND | ND | ND |
| 35 | 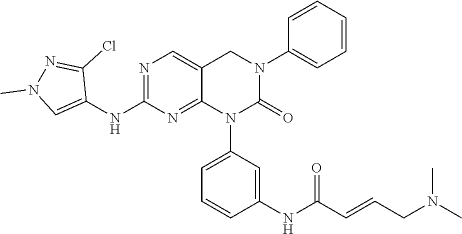 | 2166 | 12 | 55 |
| 36 | 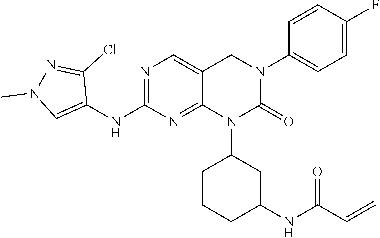 [SMILES: C=CC(=O)NC1CCCC(N2C(=O)N(c3ccc(F)cc3)Cc3cnc(Nc4cn(C)nc4Cl)nc32)C1] | 10000 | 3923 | 10000 |
| 37 | 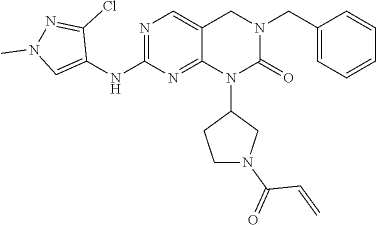 [SMILES: C=CC(=O)N1CCC(N2C(=O)N(Cc3ccccc3)Cc3cnc(Nc4cn(C)nc4Cl)nc32)C1] | 10000 | 10000 | 10000 |
| 38 | 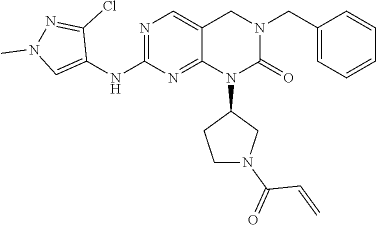 [SMILES: C=CC(=O)N1CC[C@@H](N2C(=O)N(Cc3ccccc3)Cc3cnc(Nc4cn(C)nc4Cl)nc32)C1] | 7493 | 10000 | 10000 |
| 39 | 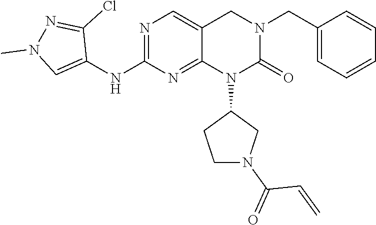 [SMILES: C=CC(=O)N1CC[C@H](N2C(=O)N(Cc3ccccc3)Cc3cnc(Nc4cn(C)nc4Cl)nc32)C1] | 10000 | 10000 | 9346 |
| 40 | 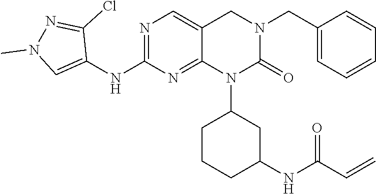 [SMILES: C=CC(=O)NC1CCCC(N2C(=O)N(Cc3ccccc3)Cc3cnc(Nc4cn(C)nc4Cl)nc32)C1] | 10000 | 1697 | 1868 |
| 41 | 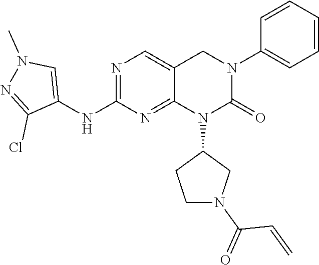 [SMILES: C=CC(=O)N1CC[C@H](N2C(=O)N(c3ccccc3)Cc3cnc(Nc4cn(C)nc4Cl)nc32)C1] | 10000 | 504 | 759 |
| 42 | 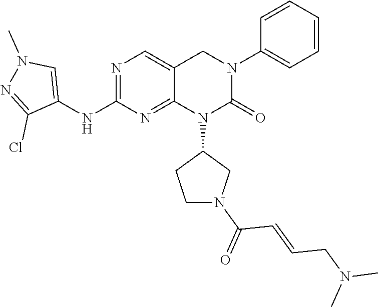 [SMILES: CN(C)C/C=C/C(=O)N1CC[C@H](N2C(=O)N(c3ccccc3)Cc3cnc(Nc4cn(C)nc4Cl)nc32)C1] | 10000 | 10000 | 10000 |
| 43 | 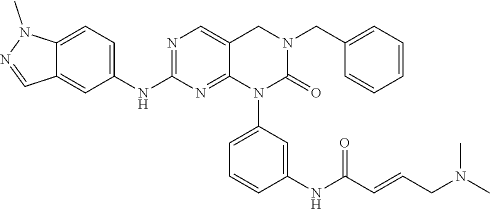 [SMILES: CN(C)C/C=C/C(=O)Nc1cccc(N2C(=O)N(Cc3ccccc3)Cc3cnc(Nc4ccc5c(cnn5C)c4)nc32)c1] | 632 | 103 | 108 |
| 44 | 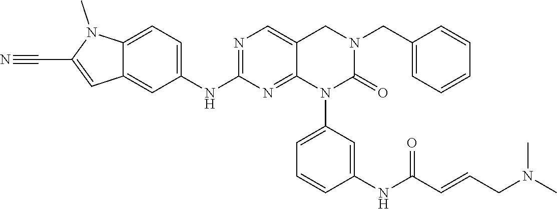 [SMILES: CN(C)C/C=C/C(=O)Nc1cccc(N2C(=O)N(Cc3ccccc3)Cc3cnc(Nc4ccc5c(c4)cc(C#N)n5C)nc32)c1] | 56 | 123 | 106 |
| 45 | 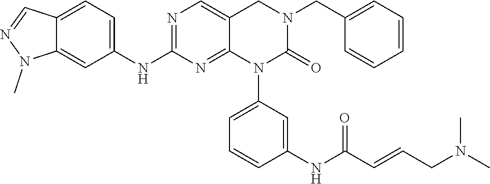 [SMILES: CN(C)C/C=C/C(=O)Nc1cccc(N2C(=O)N(Cc3ccccc3)Cc3cnc(Nc4ccc5cnn(C)c5c4)nc32)c1] | 1129 | 352 | 350 |
| 46 | 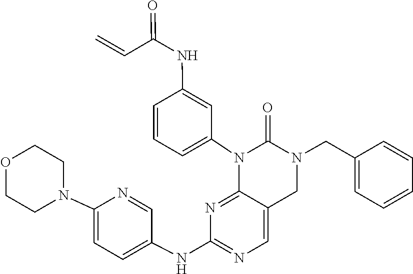 [SMILES: C=CC(=O)Nc1cccc(N2C(=O)N(Cc3ccccc3)Cc3cnc(Nc4ccc(N5CCOCC5)nc4)nc32)c1] | 1696 | 14 | 16 |
| 47 | 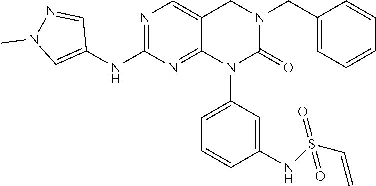 [SMILES: C=CS(=O)(=O)Nc1cccc(N2C(=O)N(Cc3ccccc3)Cc3cnc(Nc4cnn(C)c4)nc32)c1] | 10000 | 2264 | 10000 |
| 48 | 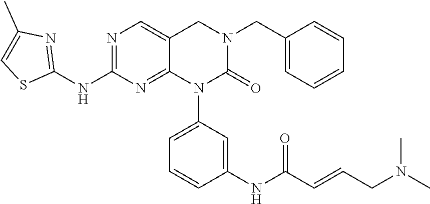 [SMILES: Cc1csc(Nc2ncc3c(n2)N(c2cccc(NC(=O)/C=C/CN(C)C)c2)C(=O)N(Cc2ccccc2)C3)n1] | 148 | 456 | 361 |
| 49 | 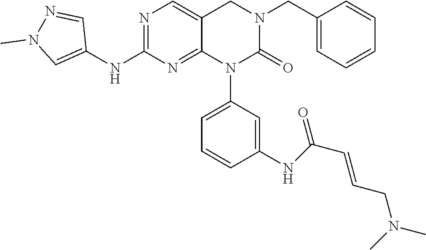 | 10000 | 351 | 476 |
| 50 | 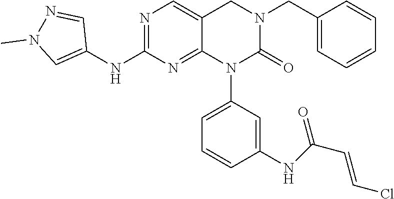 [SMILES: Cn1cc(Nc2ncc3c(n2)N(c2cccc(NC(=O)/C=C/Cl)c2)C(=O)N(Cc2ccccc2)C3)cn1] | 5383 | 1353 | 8722 |
| 51 | 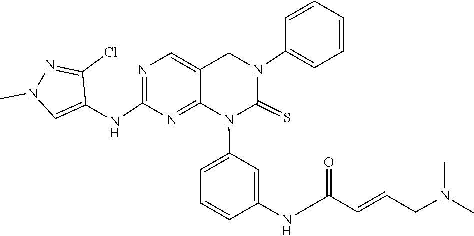 | ND | ND | ND |
| 52 | 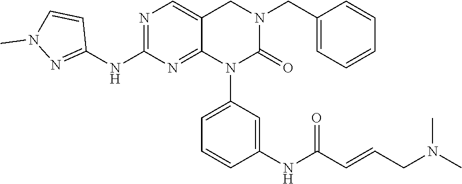 | 10000 | 244 | 341 |
| 53 | 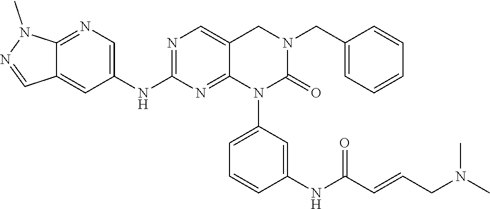 [SMILES: CN(C)C/C=C/C(=O)Nc1cccc(N2C(=O)N(Cc3ccccc3)Cc3cnc(Nc4cnc5c(cnn5C)c4)nc32)c1] | 10000 | 474 | 1052 |
| 54 | 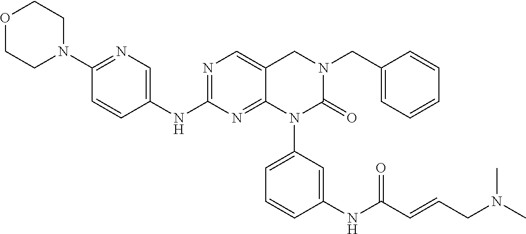 [SMILES: CN(C)C/C=C/C(=O)Nc1cccc(N2C(=O)N(Cc3ccccc3)Cc3cnc(Nc4ccc(N5CCOCC5)nc4)nc32)c1] | 10000 | 224 | 337 |
| 55 | 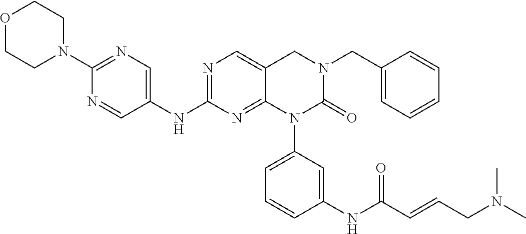 [SMILES: CN(C)C/C=C/C(=O)Nc1cccc(N2C(=O)N(Cc3ccccc3)Cc3cnc(Nc4cnc(N5CCOCC5)nc4)nc32)c1] | 10000 | 1672 | 3856 |
| 56 | 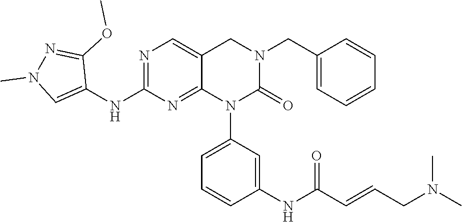 | 10000 | 65 | 107 |
| 57 | 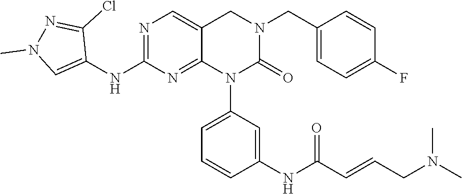 | 10000 | 115 | 476 |
| 58 | 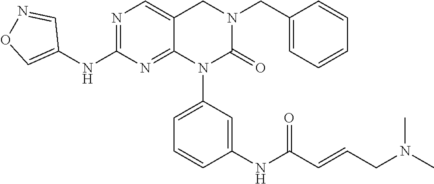 [SMILES: CN(C)C/C=C/C(=O)Nc1cccc(N2C(=O)N(Cc3ccccc3)Cc3cnc(Nc4cnoc4)nc32)c1] | 10000 | 801 | 2111 |
| 59 | 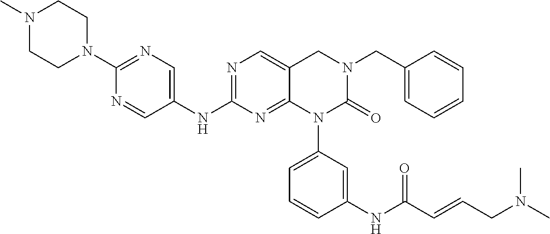 | 10000 | 8665 | 10000 |
| 60 | 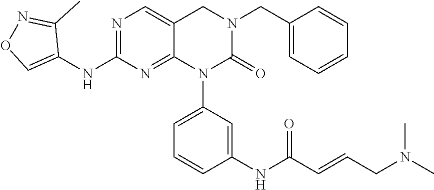 [SMILES: Cc1nocc1Nc1ncc2c(n1)N(c1cccc(NC(=O)/C=C/CN(C)C)c1)C(=O)N(Cc1ccccc1)C2] | 10000 | 1698 | 2394 |
| 61 | 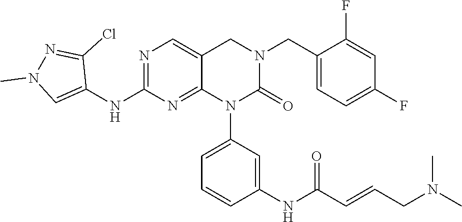 | 10000 | 58 | 105 |
| 62 | 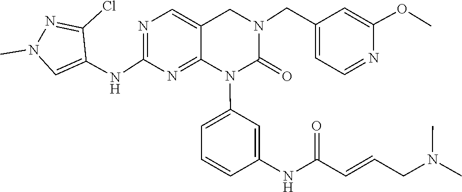 | 10000 | 2255 | 5476 |
| 63 | 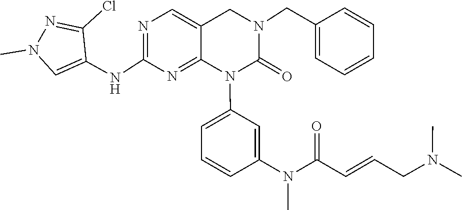 | 10000 | 532 | 1461 |
| 64 | 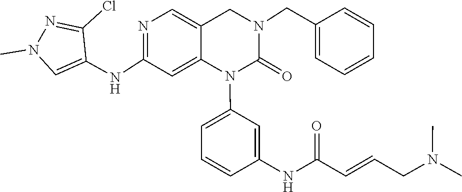 | 10000 | 809 | 1030 |
| 65 | 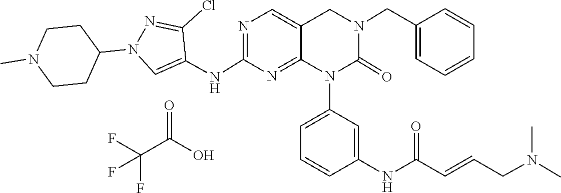 | 10000 | 964 | 1091 |
| 66 | 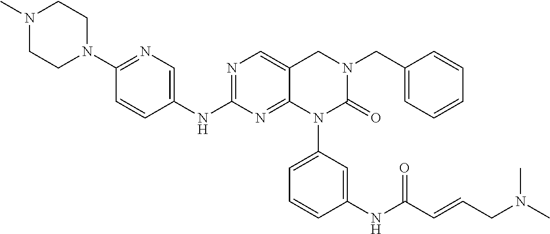 | 10000 | 3276 | 3186 |
| 67 | 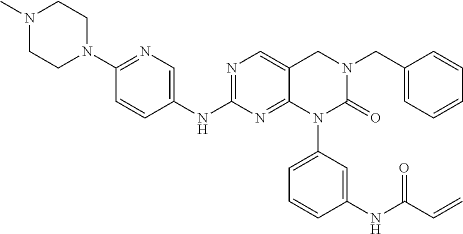 [SMILES: C=CC(=O)Nc1cccc(N2C(=O)N(Cc3ccccc3)Cc3cnc(Nc4ccc(N5CCN(C)CC5)nc4)nc32)c1] | 10000 | 41 | 84 |
| 68 | 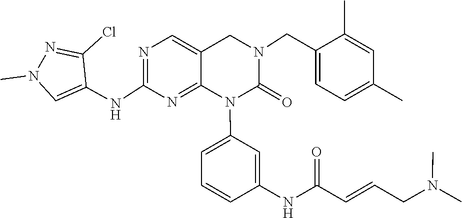 | 6024 | 356 | 798 |
| 69 | 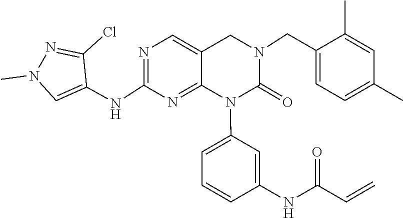 | 10000 | 333 | 349 |
| 70 | 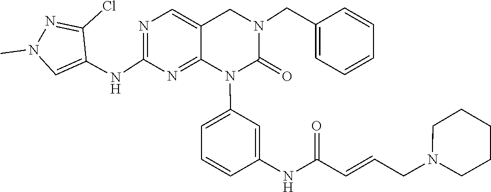 [SMILES: Cn1cc(Nc2ncc3c(n2)N(c2cccc(NC(=O)/C=C/CN4CCCCC4)c2)C(=O)N(Cc2ccccc2)C3)c(Cl)n1] | 10000 | 42 | 72 |
| 71 | 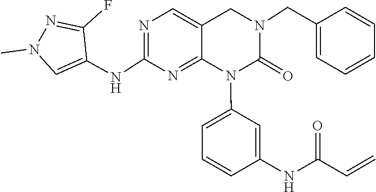 [SMILES: C=CC(=O)Nc1cccc(N2C(=O)N(Cc3ccccc3)Cc3cnc(Nc4cn(C)nc4F)nc32)c1] | 10000 | 22 | 21 |
| 72 | 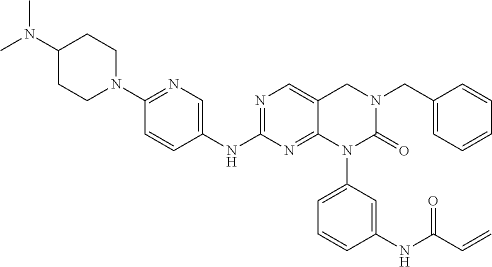 [SMILES: C=CC(=O)Nc1cccc(N2C(=O)N(Cc3ccccc3)Cc3cnc(Nc4ccc(N5CCC(N(C)C)CC5)nc4)nc32)c1] | 10000 | 100 | 296 |
| 73 | 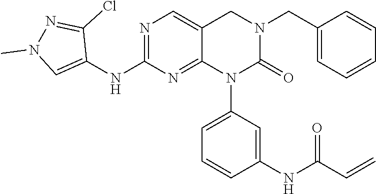 [SMILES: C=CC(=O)Nc1cccc(N2C(=O)N(Cc3ccccc3)Cc3cnc(Nc4cn(C)nc4Cl)nc32)c1] | 10000 | 4 | 20 |
| 74 | 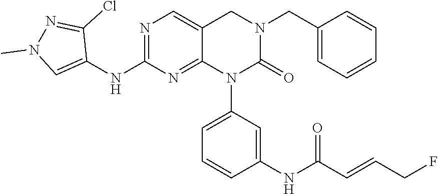 [SMILES: Cn1cc(Nc2ncc3c(n2)N(c2cccc(NC(=O)/C=C/CF)c2)C(=O)N(Cc2ccccc2)C3)c(Cl)n1] | 10000 | 11 | 25 |
| 75 | 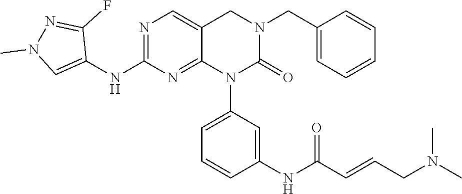 | 10000 | 394 | 980 |
| 76 | 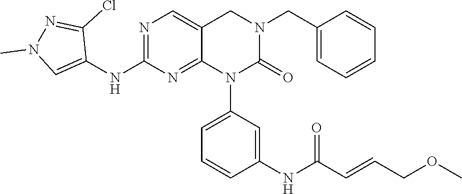 | 10000 | 336 | 342 |
| 77 | 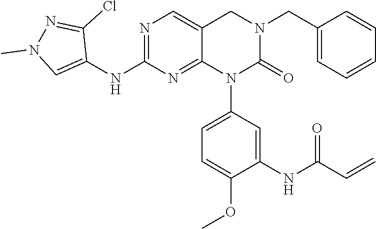 [SMILES: C=CC(=O)Nc1cc(N2C(=O)N(Cc3ccccc3)Cc3cnc(Nc4cn(C)nc4Cl)nc32)ccc1OC] | 10000 | 10000 | 9132 |
| 78 | 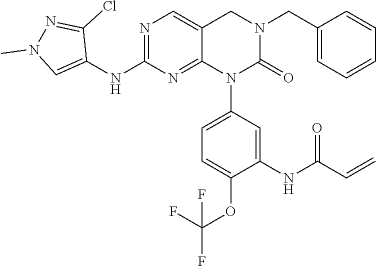 [SMILES: C=CC(=O)Nc1cc(N2C(=O)N(Cc3ccccc3)Cc3cnc(Nc4cn(C)nc4Cl)nc32)ccc1OC(F)(F)F] | 4178 | 8367 | 8779 |
| 79 | 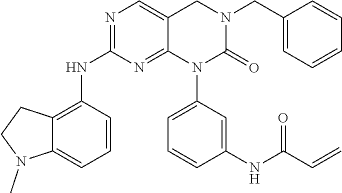 [SMILES: C=CC(=O)Nc1cccc(N2C(=O)N(Cc3ccccc3)Cc3cnc(Nc4cccc5c4CCN5C)nc32)c1] | 10000 | 89 | 107 |
| 80 | 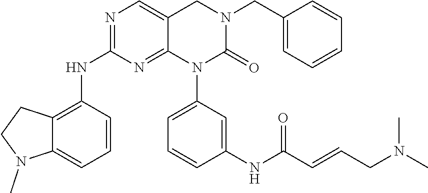 [SMILES: CN(C)C/C=C/C(=O)Nc1cccc(N2C(=O)N(Cc3ccccc3)Cc3cnc(Nc4cccc5c4CCN5C)nc32)c1] | 10000 | 343 | 806 |
| 81 | 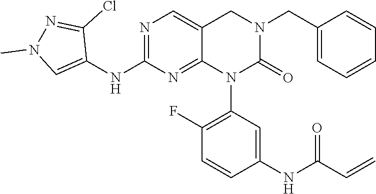 [SMILES: C=CC(=O)Nc1ccc(F)c(N2C(=O)N(Cc3ccccc3)Cc3cnc(Nc4cn(C)nc4Cl)nc32)c1] | 10000 | 27 | 35 |
| 82 | 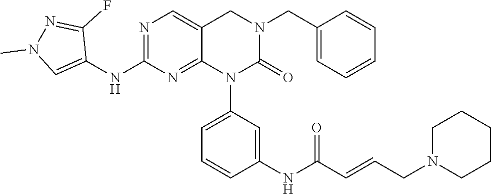 [SMILES: Cn1cc(Nc2ncc3c(n2)N(c2cccc(NC(=O)/C=C/CN4CCCCC4)c2)C(=O)N(Cc2ccccc2)C3)c(F)n1] | 10000 | 123 | 280 |
| 83 | 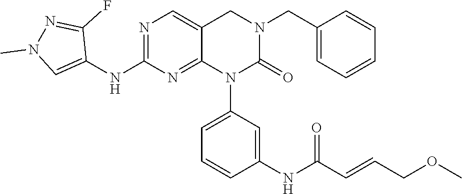 | 10000 | 1013 | 992 |
| 84 | 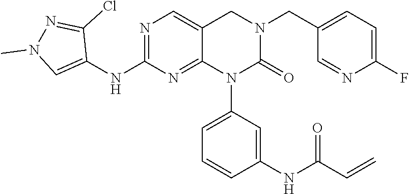 [SMILES: C=CC(=O)Nc1cccc(N2C(=O)N(Cc3ccc(F)nc3)Cc3cnc(Nc4cn(C)nc4Cl)nc32)c1] | >10000 | 347 | 374 |
| 85 | 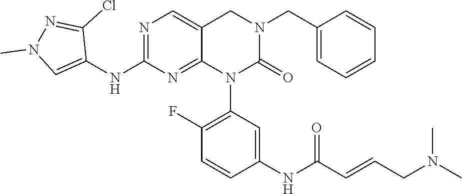 | 1276 | 94 | 123 |
| 86 | 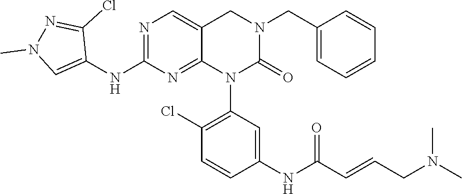 | 5441 | 694 | 900 |
| 87 | 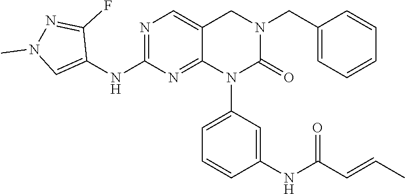 [SMILES: C/C=C/C(=O)Nc1cccc(N2C(=O)N(Cc3ccccc3)Cc3cnc(Nc4cn(C)nc4F)nc32)c1] | >10000 | 2243 | 1978 |
| 88 | 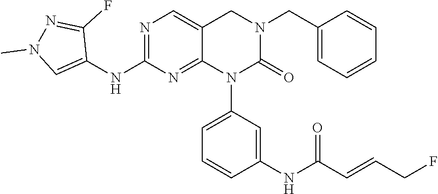 [SMILES: Cn1cc(Nc2ncc3c(n2)N(c2cccc(NC(=O)/C=C/CF)c2)C(=O)N(Cc2ccccc2)C3)c(F)n1] | >10000 | 22 | 40 |
| 89 | 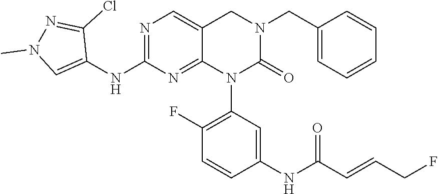 [SMILES: Cn1cc(Nc2ncc3c(n2)N(c2cc(NC(=O)/C=C/CF)ccc2F)C(=O)N(Cc2ccccc2)C3)c(Cl)n1] | ND | ND | ND |
| 90 | 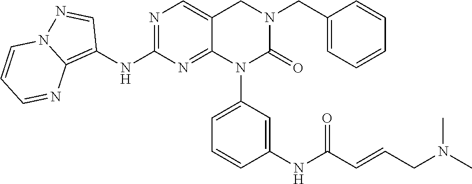 [SMILES: CN(C)C/C=C/C(=O)Nc1cccc(N2C(=O)N(Cc3ccccc3)Cc3cnc(Nc4cnn5cccnc45)nc32)c1] | 10000 | 1148 | 1852 |
| 91 | 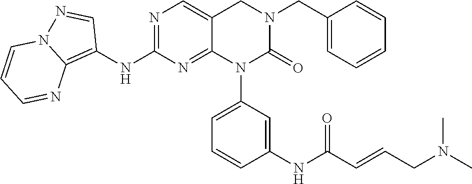 [SMILES: CN(C)C/C=C/C(=O)Nc1cccc(N2C(=O)N(Cc3ccccc3)Cc3cnc(Nc4cnn5cccnc45)nc32)c1] | 10000 | 329 | 645 |
| 92 | 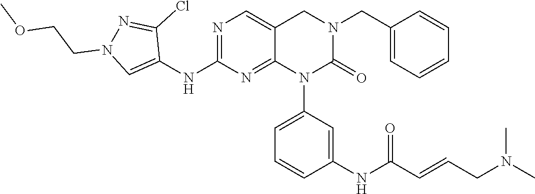 | 10000 | 385 | 228 |
| 93 | 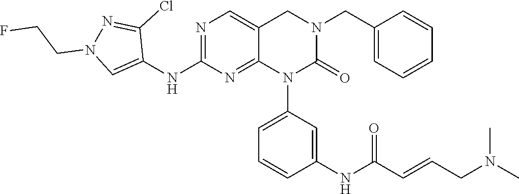 [SMILES: CN(C)C/C=C/C(=O)Nc1cccc(N2C(=O)N(Cc3ccccc3)Cc3cnc(Nc4cn(CCF)nc4Cl)nc32)c1] | 10000 | 108 | 91 |
| 94 | 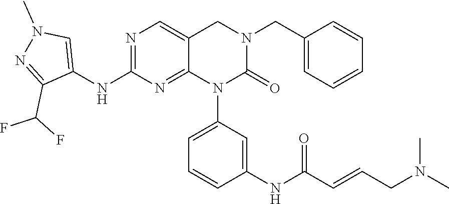 [SMILES: CN(C)C/C=C/C(=O)Nc1cccc(N2C(=O)N(Cc3ccccc3)Cc3cnc(Nc4cn(C)nc4C(F)F)nc32)c1] | 10000 | 39 | 43 |
| 95 | 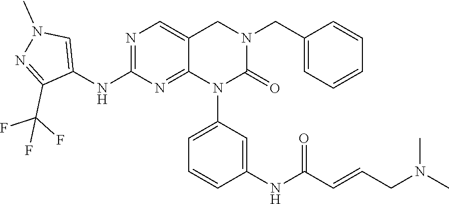 [SMILES: CN(C)C/C=C/C(=O)Nc1cccc(N2C(=O)N(Cc3ccccc3)Cc3cnc(Nc4cn(C)nc4C(F)(F)F)nc32)c1] | 6500 | 106 | 97 |
| 96 | 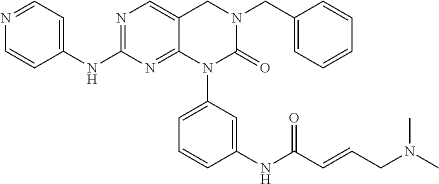 [SMILES: CN(C)C/C=C/C(=O)Nc1cccc(N2C(=O)N(Cc3ccccc3)Cc3cnc(Nc4ccncc4)nc32)c1] | 10000 | >9351 | 4074 |
| 97 | 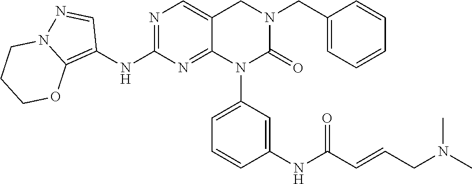 [SMILES: CN(C)C/C=C/C(=O)Nc1cccc(N2C(=O)N(Cc3ccccc3)Cc3cnc(Nc4cnn5c4OCCC5)nc32)c1] | 10000 | 2634 | 2542 |
| 98 | 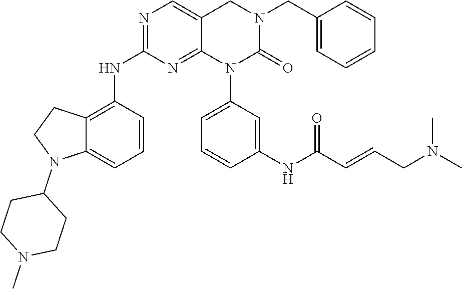 [SMILES: CN(C)C/C=C/C(=O)Nc1cccc(N2C(=O)N(Cc3ccccc3)Cc3cnc(Nc4cccc5c4CCN5C4CCN(C)CC4)nc32)c1] | 10000 | 322 | 138 |
| 99 | 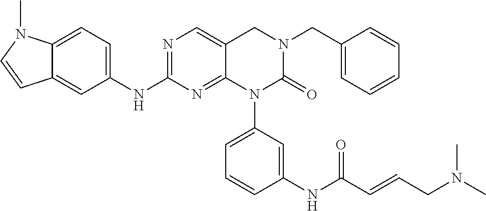 [SMILES: CN(C)C/C=C/C(=O)Nc1cccc(N2C(=O)N(Cc3ccccc3)Cc3cnc(Nc4ccc5c(ccn5C)c4)nc32)c1] | 10000 | 115 | 145 |
| 100 | 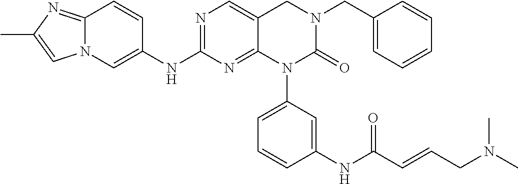 | 10000 | 2972 | 4811 |
| 101 | 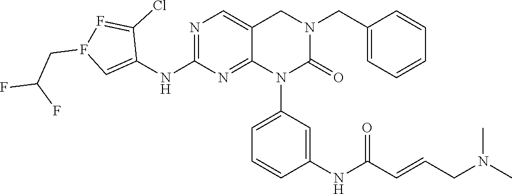 | 10000 | 100 | 176 |
| 102 | 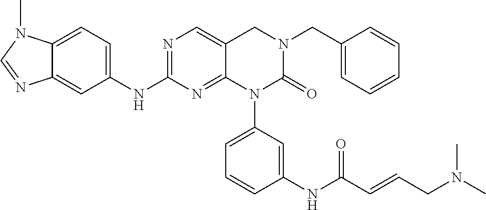 [SMILES: CN(C)C/C=C/C(=O)Nc1cccc(N2C(=O)N(Cc3ccccc3)Cc3cnc(Nc4ccc5c(c4)ncn5C)nc32)c1] | 10000 | 694 | 929 |
| 103 | 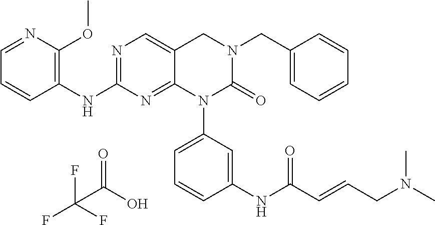 | 10000 | 269 | 224 |
| 104 | 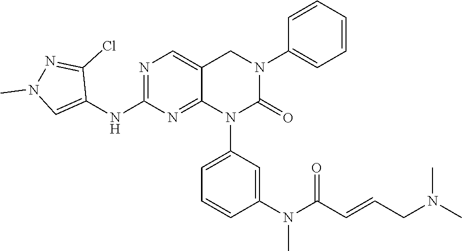 | 10000 | 255 | 885 |
| 105 | 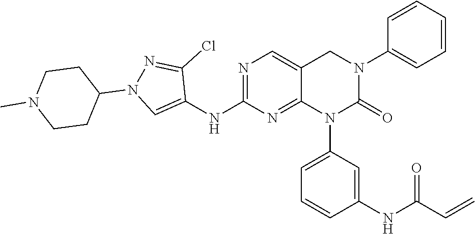 [SMILES: C=CC(=O)Nc1cccc(N2C(=O)N(c3ccccc3)Cc3cnc(Nc4cn(C5CCN(C)CC5)nc4Cl)nc32)c1] | 3597 | 12 | 14 |
| 106 | 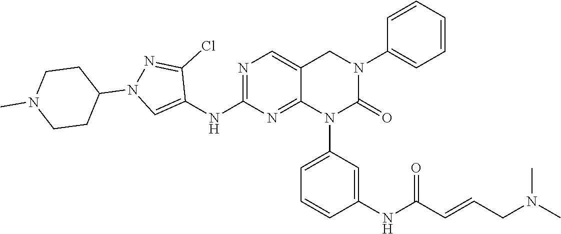 | 8927 | 926 | 1115 |
| 107 | 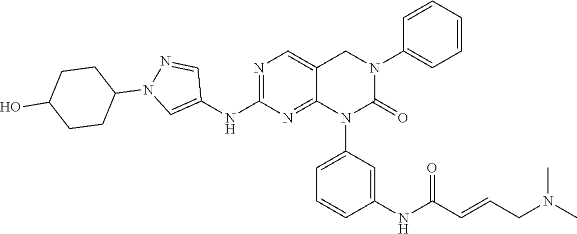 [SMILES: CN(C)C/C=C/C(=O)Nc1cccc(N2C(=O)N(c3ccccc3)Cc3cnc(Nc4cnn(C5CCC(O)CC5)c4)nc32)c1] | 10000 | 10000 | 10000 |
| 108 | 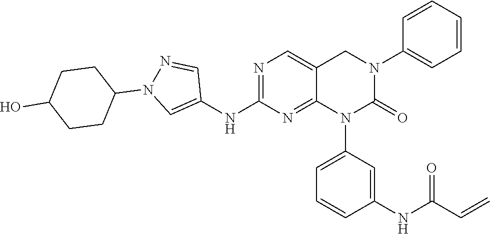 [SMILES: C=CC(=O)Nc1cccc(N2C(=O)N(c3ccccc3)Cc3cnc(Nc4cnn(C5CCC(O)CC5)c4)nc32)c1] | 10000 | 912 | 634 |
| 109 | 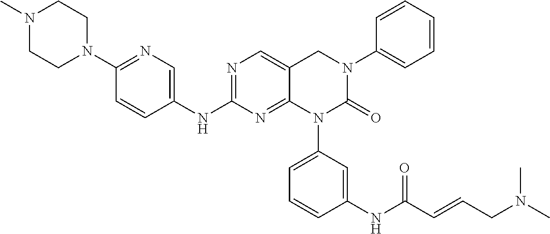 | 10000 | 2320 | 6027 |
| 110 | 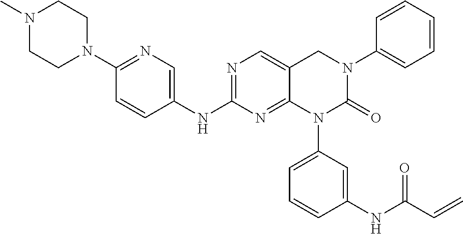 [SMILES: C=CC(=O)Nc1cccc(N2C(=O)N(c3ccccc3)Cc3cnc(Nc4ccc(N5CCN(C)CC5)nc4)nc32)c1] | 3636 | 43 | 102 |
| 111 | 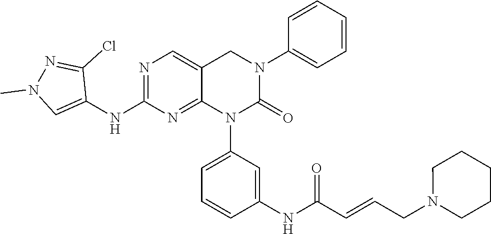 [SMILES: Cn1cc(Nc2ncc3c(n2)N(c2cccc(NC(=O)/C=C/CN4CCCCC4)c2)C(=O)N(c2ccccc2)C3)c(Cl)n1] | 10000 | 39 | 128 |
| 112 | 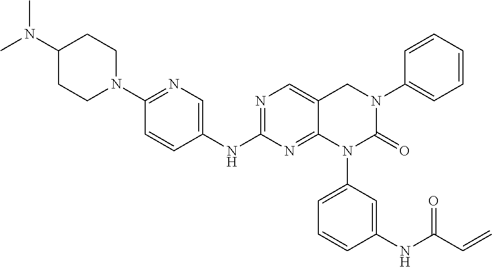 [SMILES: C=CC(=O)Nc1cccc(N2C(=O)N(c3ccccc3)Cc3cnc(Nc4ccc(N5CCC(N(C)C)CC5)nc4)nc32)c1] | 10000 | 229 | 295 |
| 113 | 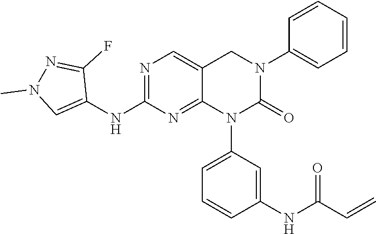 [SMILES: C=CC(=O)Nc1cccc(N2C(=O)N(c3ccccc3)Cc3cnc(Nc4cn(C)nc4F)nc32)c1] | 10000 | 4 | 17 |
| 114 | 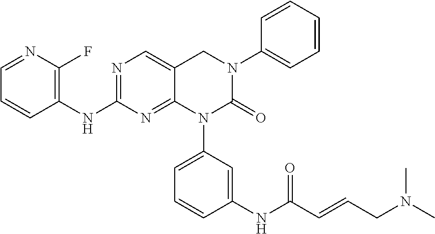 [SMILES: CN(C)C/C=C/C(=O)Nc1cccc(N2C(=O)N(c3ccccc3)Cc3cnc(Nc4cccnc4F)nc32)c1] | 10000 | 164 | 1088 |
| 115 | 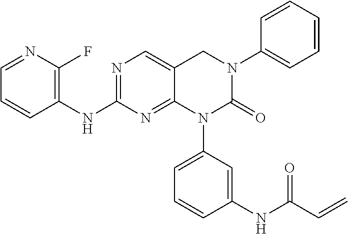 [SMILES: C=CC(=O)Nc1cccc(N2C(=O)N(c3ccccc3)Cc3cnc(Nc4cccnc4F)nc32)c1] | 10000 | 8 | 28 |
| 116 | 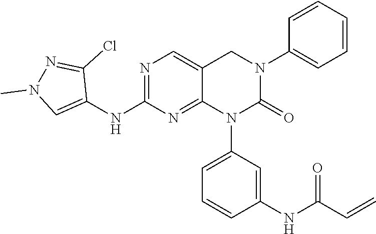 [SMILES: C=CC(=O)Nc1cccc(N2C(=O)N(c3ccccc3)Cc3cnc(Nc4cn(C)nc4Cl)nc32)c1] | 10000 | 1 | 7 |
| 117 | 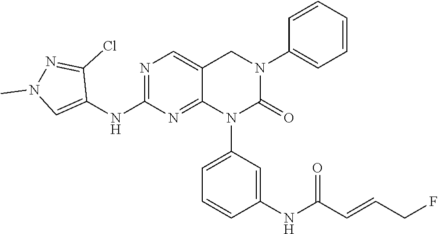 [SMILES: Cn1cc(Nc2ncc3c(n2)N(c2cccc(NC(=O)/C=C/CF)c2)C(=O)N(c2ccccc2)C3)c(Cl)n1] | 10000 | 35 | 50 |
| 118 | 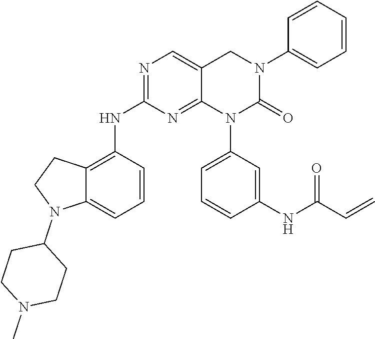 [SMILES: C=CC(=O)Nc1cccc(N2C(=O)N(c3ccccc3)Cc3cnc(Nc4cccc5c4CCN5C4CCN(C)CC4)nc32)c1] | 10000 | 32 | 110 |
| 119 | 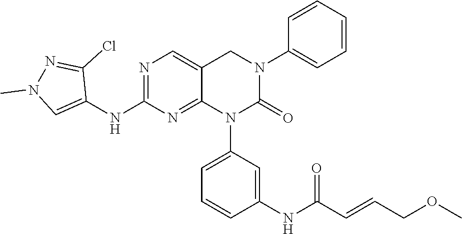 | 10000 | 98 | 140 |
| 120 | 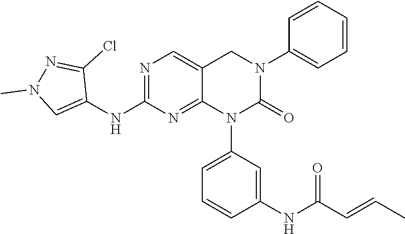 [SMILES: C/C=C/C(=O)Nc1cccc(N2C(=O)N(c3ccccc3)Cc3cnc(Nc4cn(C)nc4Cl)nc32)c1] | 10000 | 107 | 183 |
| 121 | 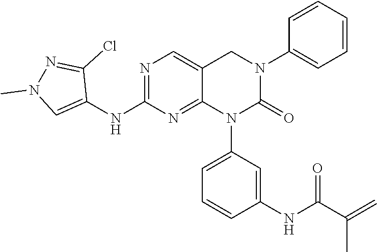 [SMILES: C=C(C)C(=O)Nc1cccc(N2C(=O)N(c3ccccc3)Cc3cnc(Nc4cn(C)nc4Cl)nc32)c1] | 10000 | 8596 | 10000 |
| 122 | 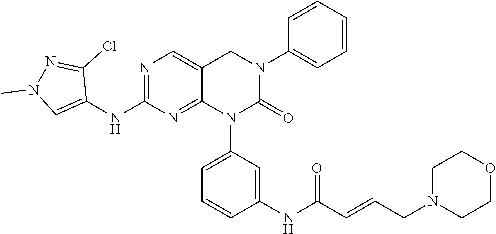 [SMILES: Cn1cc(Nc2ncc3c(n2)N(c2cccc(NC(=O)/C=C/CN4CCOCC4)c2)C(=O)N(c2ccccc2)C3)c(Cl)n1] | 10000 | 101 | 284 |
| 123 | 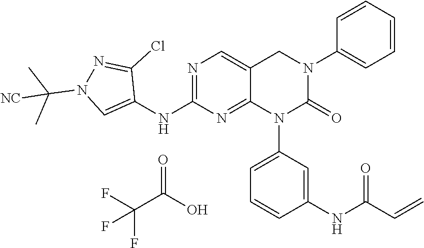 | 10000 | 176 | 300 |
| 124 | 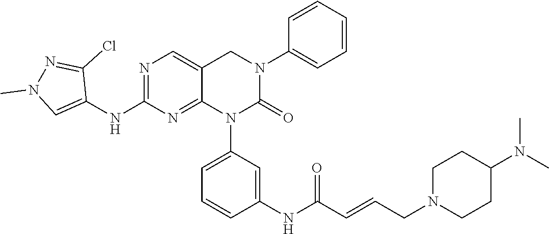 | 10000 | 2760 | 4977 |
| 125 | 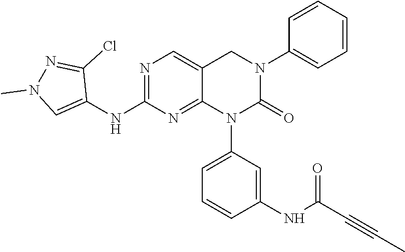 [SMILES: CC#CC(=O)Nc1cccc(N2C(=O)N(c3ccccc3)Cc3cnc(Nc4cn(C)nc4Cl)nc32)c1] | 10000 | 269 | 338 |
| 126 | 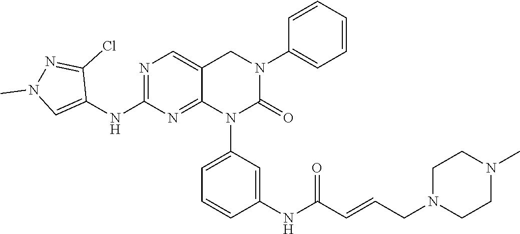 | 10000 | 1086 | 2289 |
| 127 | 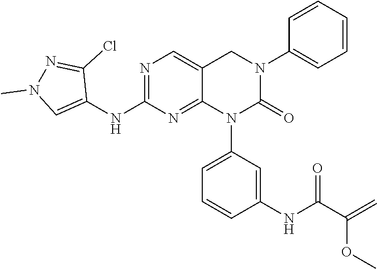 | 8170 | 10000 | 10000 |
| 128 | 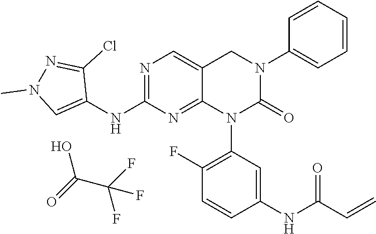 | 10000 | 11 | 12 |
| 129 | 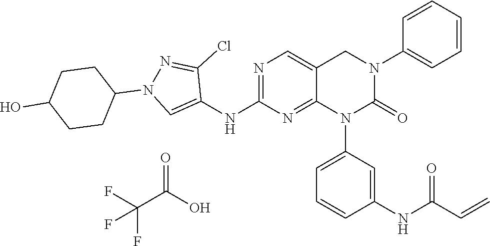 | 10000 | 157 | 333 |
| 130 | 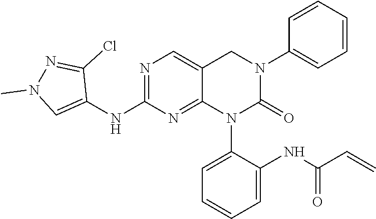 [SMILES: C=CC(=O)Nc1ccccc1N1C(=O)N(c2ccccc2)Cc2cnc(Nc3cn(C)nc3Cl)nc21] | 10000 | 10000 | 9260 |
| 131 | 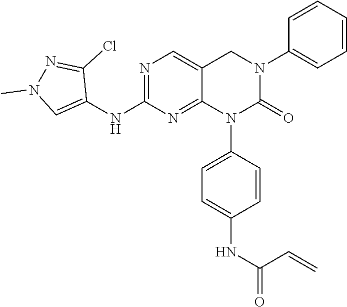 [SMILES: C=CC(=O)Nc1ccc(N2C(=O)N(c3ccccc3)Cc3cnc(Nc4cn(C)nc4Cl)nc32)cc1] | 3511 | 373 | 765 |
| 132 | 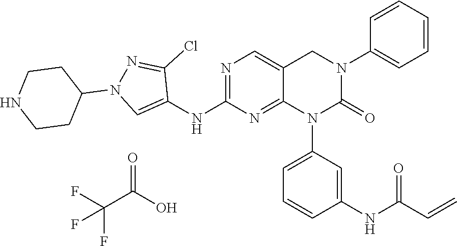 | 10000 | 960 | 973 |
| 133 (Isomer 1) | 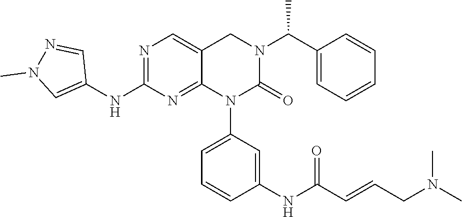 | 10000 | 1124 | 2694 |
| 134 (Isomer 2) | 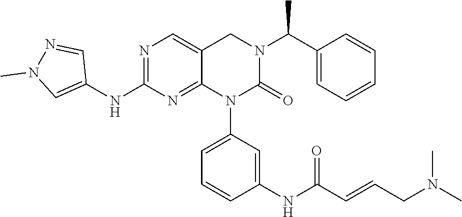 | 10000 | 109 | 103 |
| 135 | 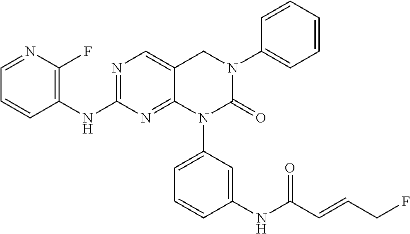 [SMILES: O=C(/C=C/CF)Nc1cccc(N2C(=O)N(c3ccccc3)Cc3cnc(Nc4cccnc4F)nc32)c1] | >10000 | 43 | 120 |
| 136 | 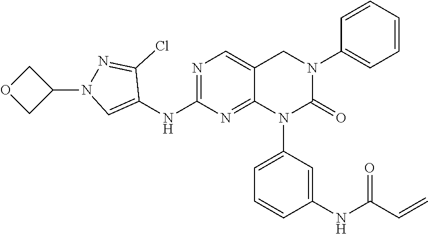 [SMILES: C=CC(=O)Nc1cccc(N2C(=O)N(c3ccccc3)Cc3cnc(Nc4cn(C5COC5)nc4Cl)nc32)c1] | >10000 | 15 | 29 |

ND: Not determined

Table 3 shows the activity of compounds of this invention in the HER2 cellular proliferation assays.

TABLE 3
Cellular proliferation data.

| | | | | HER2 WT |
|---|---|---|---|---|
| Cmpd | | YVMA IC₅₀ | BT-474 IC₅₀ | Ba/F3 IC₅₀ |
| No. | Structure | (nM) | (nM) | (nM) |
| 1 | 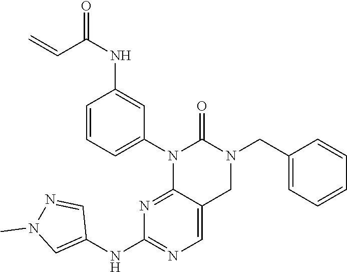 [SMILES: C=CC(=O)Nc1cccc(N2C(=O)N(Cc3ccccc3)Cc3cnc(Nc4cnn(C)c4)nc32)c1] | 9.9 | 7.7 | <0.5 |
| 6 | 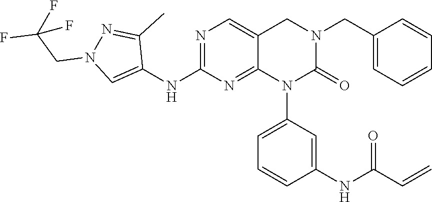 [SMILES: C=CC(=O)Nc1cccc(N2C(=O)N(Cc3ccccc3)Cc3cnc(Nc4cn(CC(F)(F)F)nc4C)nc32)c1] | 11 | ND | ND |
| 9 | 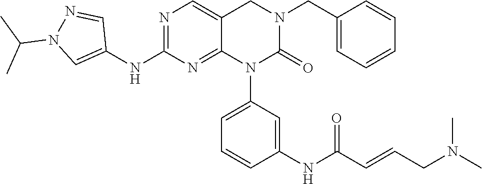 [SMILES: CC(C)n1cc(Nc2ncc3c(n2)N(c2cccc(NC(=O)/C=C/CN(C)C)c2)C(=O)N(Cc2ccccc2)C3)cn1] | 38 | ND | ND |
| 15 | 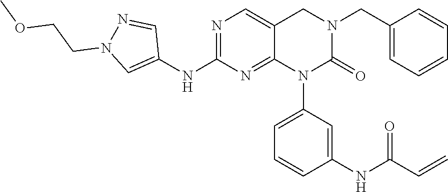 [SMILES: C=CC(=O)Nc1cccc(N2C(=O)N(Cc3ccccc3)Cc3cnc(Nc4cnn(CCOC)c4)nc32)c1] | 23 | ND | ND |
| 20 | 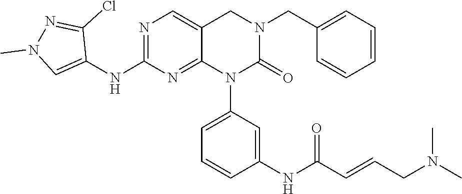 | 14.9 | 4.1 | 0.7 |
| 35 | 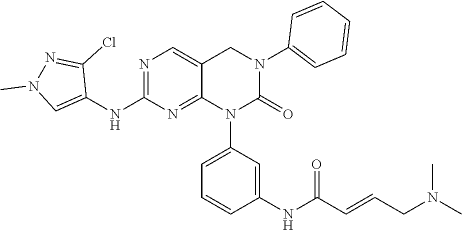 | 154 | ND | ND |
| 46 | 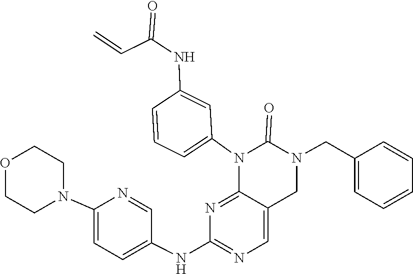 [SMILES: C=CC(=O)Nc1cccc(N2C(=O)N(Cc3ccccc3)Cc3cnc(Nc4ccc(N5CCOCC5)nc4)nc32)c1] | 67 | ND | ND |
| 49 | 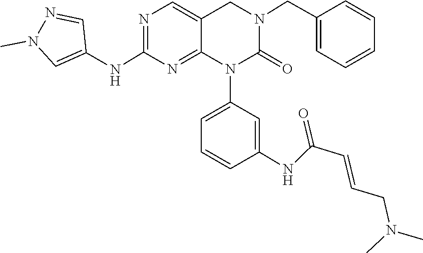 | 178 | ND | ND |
| 65 | 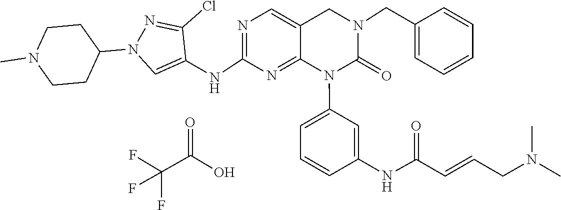 | 732 | ND | ND |
| 66 | 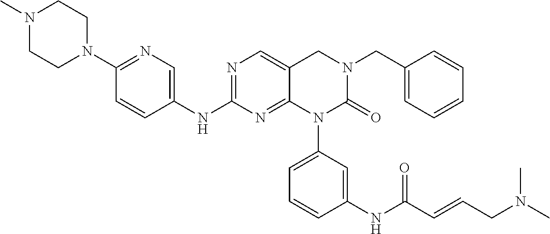 | 1764 | ND | ND |
| 67 | 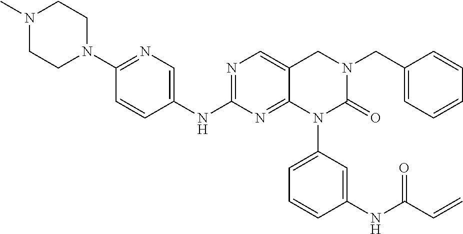 [SMILES: C=CC(=O)Nc1cccc(N2C(=O)N(Cc3ccccc3)Cc3cnc(Nc4ccc(N5CCN(C)CC5)nc4)nc32)c1] | 90 | ND | ND |
| 68 | 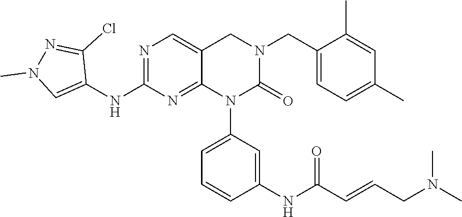 | 632 | ND | ND |
| 69 | 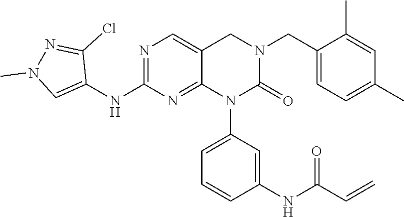 | 682 | ND | ND |
| 70 | 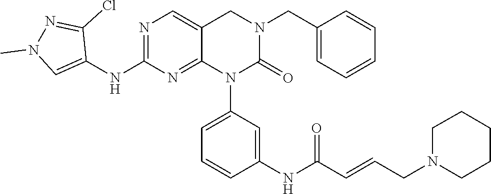 [SMILES: Cn1cc(Nc2ncc3c(n2)N(c2cccc(NC(=O)/C=C/CN4CCCCC4)c2)C(=O)N(Cc2ccccc2)C3)c(Cl)n1] | 38 | ND | ND |
| 71 | 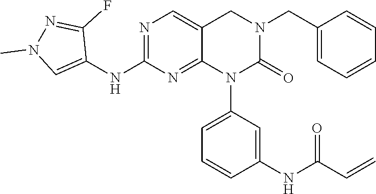 [SMILES: C=CC(=O)Nc1cccc(N2C(=O)N(Cc3ccccc3)Cc3cnc(Nc4cn(C)nc4F)nc32)c1] | 137 | ND | ND |
| 72 | 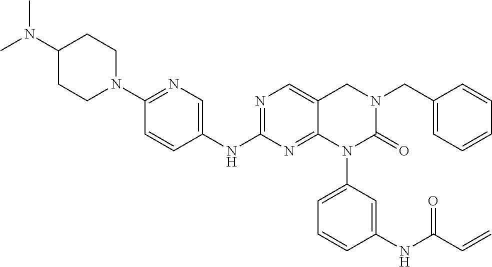 [SMILES: C=CC(=O)Nc1cccc(N2C(=O)N(Cc3ccccc3)Cc3cnc(Nc4ccc(N5CCC(N(C)C)CC5)nc4)nc32)c1] | 575 | ND | ND |
| 73 | 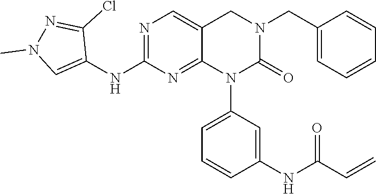 [SMILES: C=CC(=O)Nc1cccc(N2C(=O)N(Cc3ccccc3)Cc3cnc(Nc4cn(C)nc4Cl)nc32)c1] | 49 | ND | ND |
| 74 | 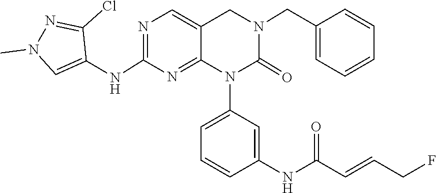 [SMILES: Cn1cc(Nc2ncc3c(n2)N(c2cccc(NC(=O)/C=C/CF)c2)C(=O)N(Cc2ccccc2)C3)c(Cl)n1] | 80 | ND | ND |
| 75 | 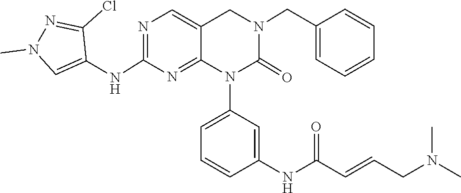 | 125 | ND | ND |
| 79 | 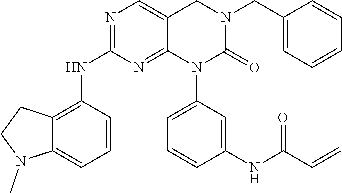 [SMILES: C=CC(=O)Nc1cccc(N2C(=O)N(Cc3ccccc3)Cc3cnc(Nc4cccc5c4CCN5C)nc32)c1] | 338 | ND | ND |
| 80 | 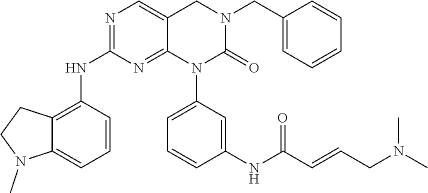 [SMILES: CN(C)C/C=C/C(=O)Nc1cccc(N2C(=O)N(Cc3ccccc3)Cc3cnc(Nc4cccc5c4CCN5C)nc32)c1] | 296 | ND | ND |
| 81 | 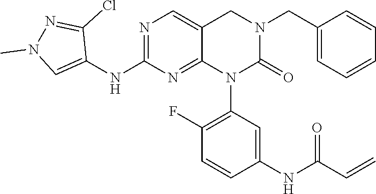 [SMILES: C=CC(=O)Nc1ccc(F)c(N2C(=O)N(Cc3ccccc3)Cc3cnc(Nc4cn(C)nc4Cl)nc32)c1] | 13 | ND | ND |
| 82 | 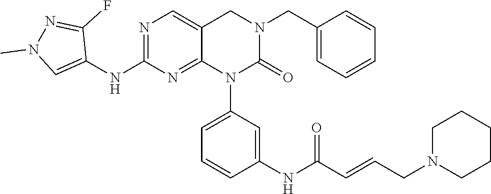 [SMILES: Cn1cc(Nc2ncc3c(n2)N(c2cccc(NC(=O)/C=C/CN4CCCCC4)c2)C(=O)N(Cc2ccccc2)C3)c(F)n1] | 40 | ND | ND |
| 83 | 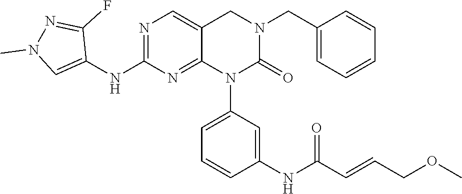 | 740 | ND | ND |
| 84 | 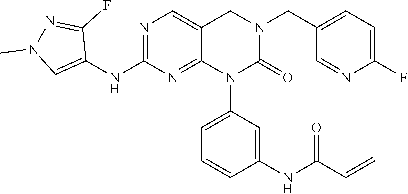 [SMILES: C=CC(=O)Nc1cccc(N2C(=O)N(Cc3ccc(F)nc3)Cc3cnc(Nc4cn(C)nc4F)nc32)c1] | 452 | ND | ND |
| 85 | 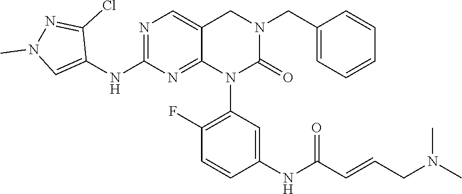 | 38 | ND | ND |
| 88 | 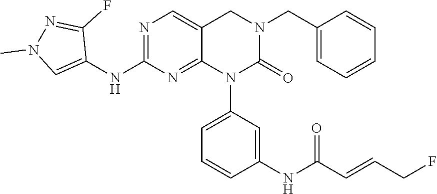 [SMILES: Cn1cc(Nc2ncc3c(n2)N(c2cccc(NC(=O)/C=C/CF)c2)C(=O)N(Cc2ccccc2)C3)c(F)n1] | 101 | ND | ND |
| 106 | 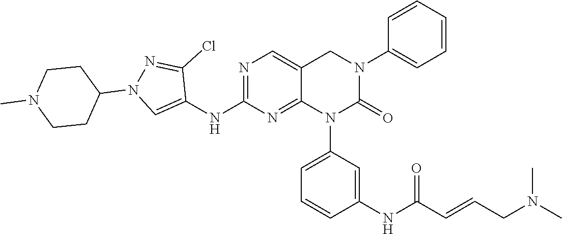 | 995 | ND | ND |
| 107 | 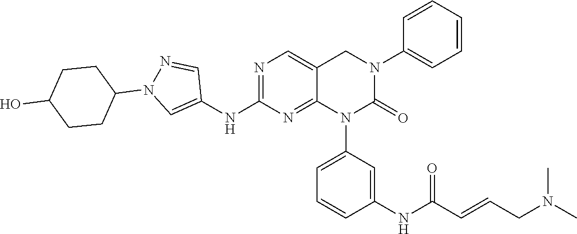 [SMILES: CN(C)C/C=C/C(=O)Nc1cccc(N2C(=O)N(c3ccccc3)Cc3cnc(Nc4cnn(C5CCC(O)CC5)c4)nc32)c1] | 10000 | ND | ND |
| 108 | 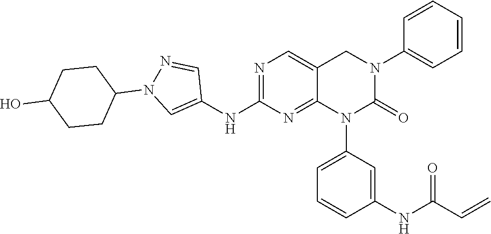 [SMILES: C=CC(=O)Nc1cccc(N2C(=O)N(c3ccccc3)Cc3cnc(Nc4cnn(C5CCC(O)CC5)c4)nc32)c1] | 2021 | ND | ND |
| 109 | 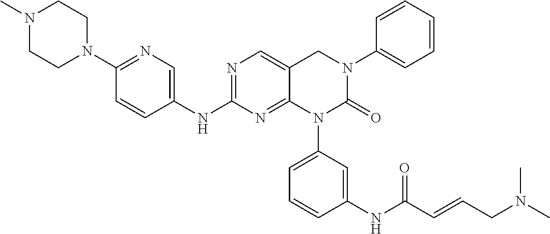 | 3298 | ND | ND |
| 110 | 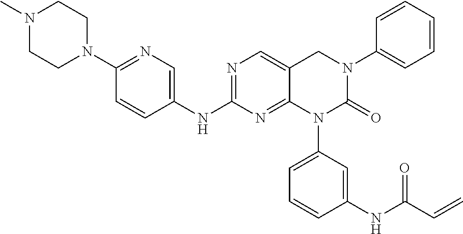 [SMILES: C=CC(=O)Nc1cccc(N2C(=O)N(c3ccccc3)Cc3cnc(Nc4ccc(N5CCN(C)CC5)nc4)nc32)c1] | 103 | ND | ND |
| 111 | 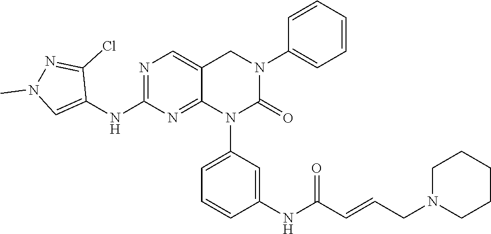 [SMILES: Cn1cc(Nc2ncc3c(n2)N(c2cccc(NC(=O)/C=C/CN4CCCCC4)c2)C(=O)N(c2ccccc2)C3)c(Cl)n1] | 64 | ND | ND |
| 112 | 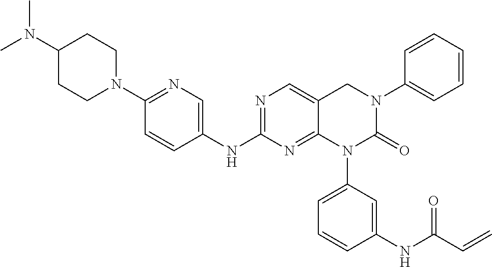 [SMILES: C=CC(=O)Nc1cccc(N2C(=O)N(c3ccccc3)Cc3cnc(Nc4ccc(N5CCC(N(C)C)CC5)nc4)nc32)c1] | 354 | ND | ND |
| 113 | 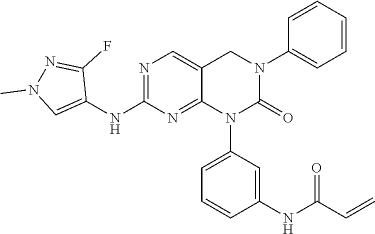 [SMILES: C=CC(=O)Nc1cccc(N2C(=O)N(c3ccccc3)Cc3cnc(Nc4cn(C)nc4F)nc32)c1] | 68 | ND | ND |
| 114 | 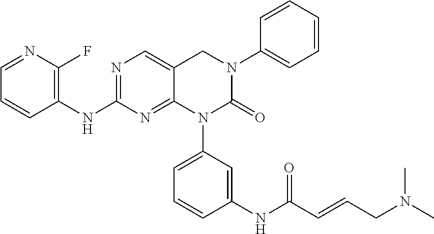 [SMILES: CN(C)C/C=C/C(=O)Nc1cccc(N2C(=O)N(c3ccccc3)Cc3cnc(Nc4cccnc4F)nc32)c1] | 1151 | ND | ND |
| 115 | 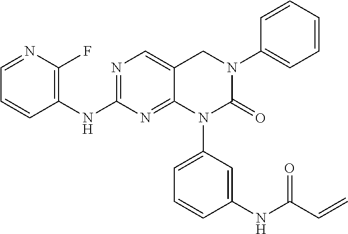 [SMILES: C=CC(=O)Nc1cccc(N2C(=O)N(c3ccccc3)Cc3cnc(Nc4cccnc4F)nc32)c1] | 188 | ND | ND |
| 116 | 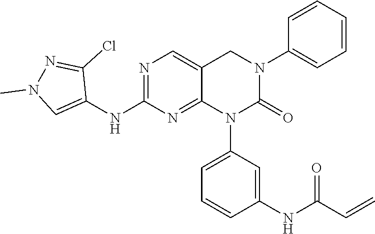 [SMILES: C=CC(=O)Nc1cccc(N2C(=O)N(c3ccccc3)Cc3cnc(Nc4cn(C)nc4Cl)nc32)c1] | 24 | ND | ND |
| 117 | 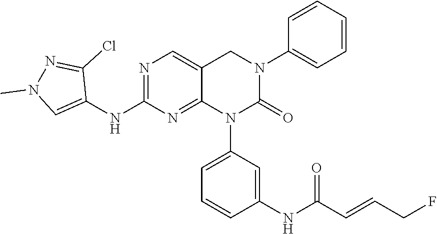 [SMILES: Cn1cc(Nc2ncc3c(n2)N(c2cccc(NC(=O)/C=C/CF)c2)C(=O)N(c2ccccc2)C3)c(Cl)n1] | 75 | ND | ND |
| 118 | 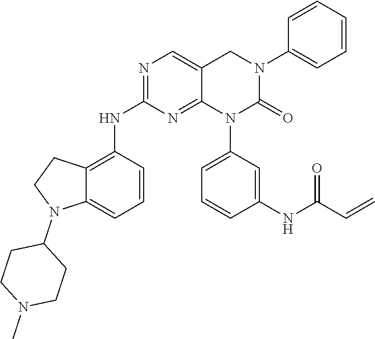 [SMILES: C=CC(=O)Nc1cccc(N2C(=O)N(c3ccccc3)Cc3cnc(Nc4cccc5c4CCN5C4CCN(C)CC4)nc32)c1] | 103 | ND | ND |
| 119 | 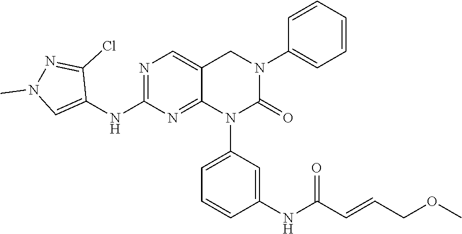 | 144 | ND | ND |
| 120 | 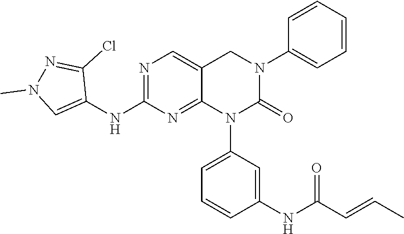 [SMILES: C/C=C/C(=O)Nc1cccc(N2C(=O)N(c3ccccc3)Cc3cnc(Nc4cn(C)nc4Cl)nc32)c1] | 158 | ND | ND |
| 121 | 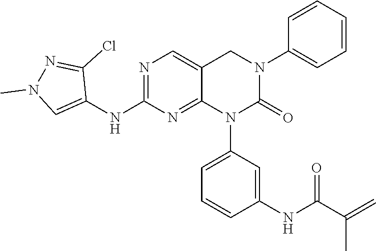 [SMILES: C=C(C)C(=O)Nc1cccc(N2C(=O)N(c3ccccc3)Cc3cnc(Nc4cn(C)nc4Cl)nc32)c1] | 883 | ND | ND |
| 122 | 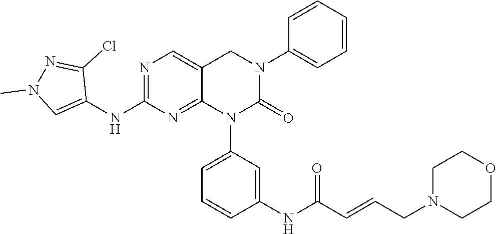 [SMILES: Cn1cc(Nc2ncc3c(n2)N(c2cccc(NC(=O)/C=C/CN4CCOCC4)c2)C(=O)N(c2ccccc2)C3)c(Cl)n1] | 132 | ND | ND |
| 123 | 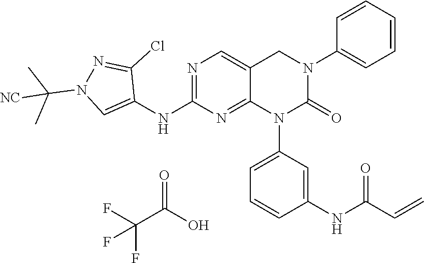 [SMILES: C=CC(=O)Nc1cccc(N2C(=O)N(c3ccccc3)Cc3cnc(Nc4cn(C(C)(C)C#N)nc4Cl)nc32)c1 ; O=C(O)C(F)(F)F] | 319 | ND | ND |
| 124 | 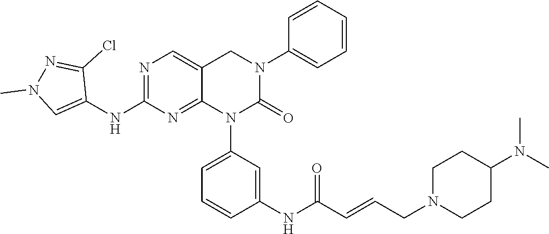 | 1485 | ND | ND |
| 125 | 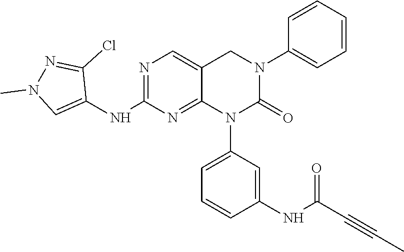 [SMILES: CC#CC(=O)Nc1cccc(N2C(=O)N(c3ccccc3)Cc3cnc(Nc4cn(C)nc4Cl)nc32)c1] | 445 | ND | ND |
| 128 | 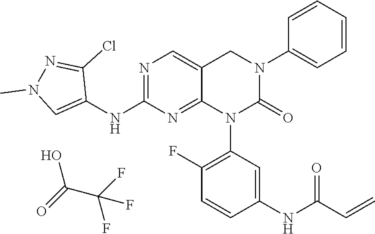 | 24 | ND | ND |
| 129 | 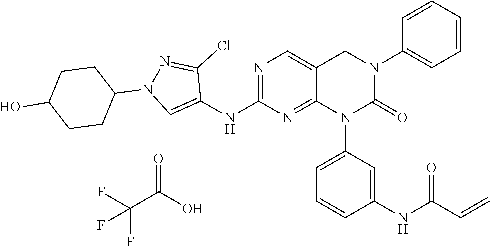 | 312 | ND | ND |
| 131 | 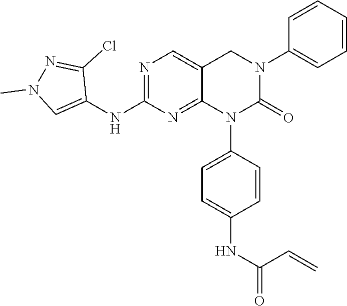 [SMILES: C=CC(=O)Nc1ccc(N2C(=O)N(c3ccccc3)Cc3cnc(Nc4cn(C)nc4Cl)nc32)cc1] | 1131 | ND | ND |
| 135 | 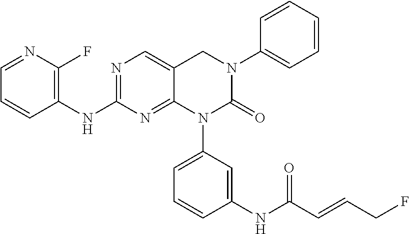 [SMILES: O=C(/C=C/CF)Nc1cccc(N2C(=O)N(c3ccccc3)Cc3cnc(Nc4cccnc4F)nc32)c1] | 294 | ND | ND |
| 136 | 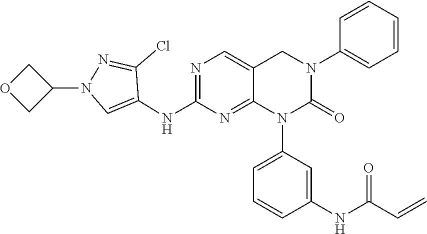 [SMILES: C=CC(=O)Nc1cccc(N2C(=O)N(c3ccccc3)Cc3cnc(Nc4cn(C5COC5)nc4Cl)nc32)c1] | 63 | ND | ND |

ND: Not determined

What is claimed is:

1. A compound of Formula I: 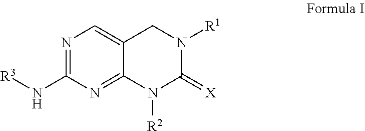
or a pharmaceutically acceptable salt thereof, wherein:
X is O or S;
R¹ is —(C(R⁴)₂)ₙR⁵, wherein R⁵ is substituted with 0, 1, or 2 R^{5′};
n is 0, 1, 2, or 3;
each R⁴ is independently hydrogen, alkyl, halo, haloalkyl, hydroxy, alkoxy, or heteroalkyl;
R⁵ is aryl or C-linked heteroaryl;
each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
each R⁶ is independently alkyl, cycloalkyl, aryl, or heteroaryl;
R² is aryl, heteroaryl, cycloalkyl, or heterocycloalkyl, wherein the aryl, heteroaryl, cycloalkyl, or heterocycloalkyl is substituted with at least one R⁷ and 0, 1, or 2 R⁸;
each R⁷ is independently 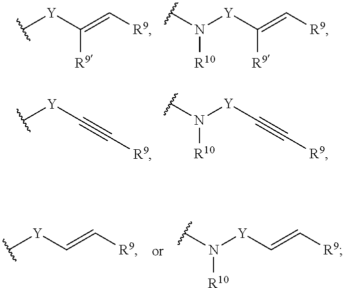
Y is —C(═O)—, —S(═O)—, or —S(═O)₂—;
R⁹ and R^{9′} are independently hydrogen, halo, alkyl, haloalkyl, cycloalkyl, heteroalkyl, or (alkyl)heterocycloalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
each R¹¹ is independently alkyl, cycloalkyl, aryl, or heteroaryl;
R³ is heteroaryl substituted with 0, 1, 2, or 3 R¹²;
each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
each R¹³ is independently alkyl, cycloalkyl, aryl, or heteroaryl;
each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

2. A compound of Formula I: 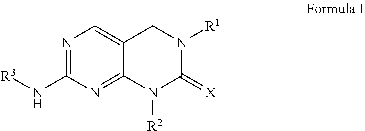
or a pharmaceutically acceptable salt thereof, wherein:
X is O or S;
R¹ is —(C(R⁴)₂)ₙR⁵, wherein R⁵ is substituted with 0, 1, or 2 R^{5′};
n is 0, 1, 2, or 3;
each R⁴ is independently hydrogen, alkyl, halo, haloalkyl, hydroxy, alkoxy, or heteroalkyl;
R⁵ is aryl or C-linked heteroaryl;
each R^{5′} is independently aryl, heteroaryl, alkyl, cycloalkyl, heterocycloalkyl, halo, heteroalkyl, haloalkyl, cyano, hydroxy, amino, —N(R⁶)₂, —S(═O)₂alkyl, —S(═O)₂aryl, —S(═O)₂heteroaryl, or alkoxy;
each R⁶ is independently alkyl, cycloalkyl, aryl, or heteroaryl;
R² is aryl, heteroaryl, cycloalkyl, or heterocycloalkyl, wherein the aryl, cycloalkyl, or heterocycloalkyl is substituted with at least one R⁷ and 0, 1, or 2 R⁸;
each R⁷ is independently 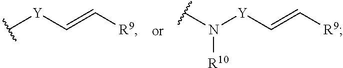
Y is —C(═O)—, —S(═O)—, or —S(═O)₂—;
R⁹ is hydrogen, halo, alkyl, cycloalkyl, or heteroalkyl;
R¹⁰ is hydrogen, alkyl, haloalkyl, or cycloalkyl;
each R⁸ is independently aryl, heteroaryl, alkyl, cycloalkyl, heterocycloalkyl, halo, heteroalkyl, haloalkyl, cyano, hydroxy, amino, —N(R¹¹)₂, —S(═O)₂alkyl, —S(═O)₂aryl, —S(═O)₂heteroaryl, or alkoxy;
each R¹¹ is independently alkyl, cycloalkyl, aryl, or heteroaryl;
R³ is heteroaryl substituted with 0, 1, 2, or 3 R¹²;
each R¹² is independently aryl, heteroaryl, alkyl, heteroalkyl, haloalkyl, halo, cyano, alkoxy, heterocycloalkyl, —N(R¹³)₂, —S(═O)₂NH₂, —S(═O)₂alkyl, —S(═O)₂aryl, —S(═O)₂heteroaryl, or cycloalkyl, wherein the aryl, heteroaryl, heterocycloalkyl, or cycloalkyl are each independently substituted with 0, 1, or 2 R¹⁴;
each R¹³ is independently alkyl, cycloalkyl, aryl, or heteroaryl;
each R¹⁴ is independently aryl, heteroaryl, alkyl, cycloalkyl, heterocycloalkyl, halo, heteroalkyl, haloalkyl, cyano, hydroxy, amino, —N(R¹⁵)₂, —S(═O)₂alkyl, —S(═O)₂aryl, —S(═O)₂heteroaryl, or alkoxy; and
each R¹⁵ is independently alkyl, cycloalkyl, aryl, or heteroaryl.

3. The compound of claim 1 or claim 2, wherein n is 0 or 1.

4. The compound of any one of claims 1 to 3, wherein R⁵ is phenyl, naphthyl, anthracenyl, phenanthrenyl, C-linked pyridyl, C-linked pyrimidinyl, C-linked pyrazolyl, or C-linked imidazolyl.

5. The compound of any one of claims 1 to 4, wherein R⁵ is unsubstituted.

6. The compound of any one of claims 1 to 4, wherein R⁵ is substituted with 1 or 2 R^{5′}.

7. The compound of any one of claims 1 to 6, wherein each R⁴ is independently hydrogen, alkyl, halo, haloalkyl, or alkoxy.

8. The compound of any one of claims 1 to 7, wherein each R⁴ is independently hydrogen, methyl, ethyl, n-propyl, iso-propyl, n-butyl, iso-butyl, sec-butyl, tert-butyl, fluoro, chloro, trifluoromethyl, trifluoroethyl, pentafluoroethyl, methoxy, ethoxy, or trifluoromethoxy.

9. The compound of any one of claims 1 to 8, wherein each R⁴ is independently hydrogen, methyl, fluoro, trifluoromethyl, methoxy, or trifluoromethoxy.

10. The compound of any one of claims 1 to 4 or 6 to 9, wherein each R^{5′} is independently aryl, heteroaryl, alkyl, heterocycloalkyl, halo, cyano, hydroxy, —N(R⁶)₂, or alkoxy.

11. The compound of any one of claims 1 to 4 or 6 to 10, wherein each R^{5′} is independently phenyl, pyrrolyl, imidazolyl, thiazolyl, isothiazolyl, oxazolyl, isoxazolyl, pyridinyl, pyrimidinyl, methyl, ethyl, tert-butyl, pyrrolidinyl, piperidinyl, piperazinyl, morpholinyl, fluoro, chloro, cyano, hydroxy, —N(R⁶)₂, methoxy, ethoxy, or trifluoromethoxy.

12. The compound of any one of claims 1 to 4 or 6 to 11, wherein each R^{5′} is independently phenyl, imidazolyl, pyridinyl, methyl, tert-butyl, pyrrolidinyl, morpholinyl, fluoro, cyano, hydroxy, —N(R⁶)₂, or methoxy.

13. The compound of any one of claims 1 to 4 or 6 to 12, wherein each R⁶ is independently alkyl or aryl.

14. The compound of any one of claims 1 to 4 or 6 to 13, wherein each R⁶ is independently methyl, ethyl, iso-propyl, tert-butyl, phenyl, or naphthyl.

15. The compound of any one of claims 1 to 4 or 6 to 14, wherein each R⁶ is independently methyl or phenyl.

16. The compound of any one of claims 1 to 15, wherein X is S.

17. The compound of any one of claims 1 to 15, wherein X is O.

18. The compound of any one of claims 1 to 17, wherein R² is monocyclic.

19. The compound of any one of claims 1 to 18, wherein R² is phenyl, cyclopropyl, cyclobutyl, cyclopentyl, cyclohexyl, pyrrolyl, imidazolyl, pyrazolyl, triazolyl, tetrazolyl, thiazolyl, isothiazolyl, oxazolyl, isoxazolyl, pyridinyl, pyrimidinyl, pyridazinyl, pyrazinyl, or triazinyl.

20. The compound of any one of claims 1 to 19, wherein R² is phenyl, cyclohexyl, or pyrrolyl.

21. The compound of any one of claims 1 or 3 to 20, wherein R⁷ is

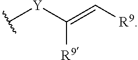

22. The compound of any one of claims 1 or 3 to 20, wherein R⁷ is

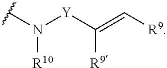

23. The compound of any one of claims 1 to 20, wherein R⁷ is

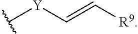

24. The compound of any one of claims 1 to 20, wherein R⁷ is

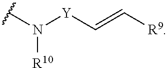

25. The compound of any one of claims 1 to 24, wherein Y is —C(═O)—.

26. The compound of any one of claims 1 to 24, wherein Y is —S(═O)₂—.

27. The compound of any one of claims 1 or 3 to 26, wherein R⁹ and R^{9′} are independently hydrogen, halo, alkyl, heteroalkyl, haloalkyl, or (alkyl)heterocycloalkyl.

28. The compound of any one of claims 1 to 26, wherein R⁹ is hydrogen, halo, or heteroalkyl.

29. The compound of any one of claims 1 or 3 to 27, wherein R⁹ and R^{9′} are independently hydrogen, fluoro, chloro, methyl, hydroxyethyl, methoxyethyl, methoxymethyl, dimethylaminomethyl, 1-piperidinylmethyl, 1-morpholinylmethyl, or fluoromethyl.

30. The compound of any one of claims 1 to 27, wherein R⁹ is hydrogen, fluoro, chloro, hydroxyethyl, or methoxyethyl.

31. The compound of any one of claims 1 to 20, 22, or 24 to 29, wherein R¹⁰ is hydrogen, methyl, ethyl n-propyl, iso-propyl, n-butyl, sec-butyl, tert-butyl, trifluoromethyl, or cyclopropyl.

32. The compound of any one of claims 1 to 20, 22, or 24 to 31, wherein R¹⁰ is hydrogen or methyl.

33. The compound of any one of claims 1 to 32, wherein R² is substituted with 1 or 2 R⁸.

34. The compound of any one of claims 1 to 33, wherein each R⁸ is independently methyl, ethyl, n-propyl, iso-propyl, n-butyl, iso-butyl, sec-butyl, tert-butyl, fluoro, chloro, heteroalkyl, cyano, hydroxy, amino, —N(R¹¹)₂, methoxy, ethoxy, or trifluoromethoxy.

35. The compound of any one of claims 1 to 34, wherein each R⁸ is independently methyl, ethyl, iso-propyl, tert-butyl, fluoro, chloro, —N(R¹¹)₂, hydroxyethyl, methoxyethyl, or cyano.

36. The compound of any one of claims 1 to 35, wherein each R¹¹ is independently alkyl or aryl.

37. The compound of any one of claims 1 to 36, wherein each R¹¹ is independently methyl, ethyl, n-propyl, iso-propyl, n-butyl, iso-butyl, sec-butyl, tert-butyl, phenyl, naphthyl, anthracenyl, or phenanthrenyl.

38. The compound of any one of claims 1 to 37, wherein each R¹¹ is independently methyl, ethyl, iso-propyl, tert-butyl, phenyl, or naphthyl.

39. The compound of any one of claims 1 to 38, wherein each R¹¹ is independently methyl or phenyl.

40. The compound of any one of claims 1 to 32, wherein R² is unsubstituted.

41. The compound of any one of claims 1 to 40, wherein R³ is pyrrolyl, imidazolyl, pyrazolyl, triazolyl, tetrazolyl, indolyl, indazolyl, benzimidazolyl, azaindolyl, thiazolyl, isothiazolyl, oxazolyl, isoxazolyl, pyridinyl, pyrimidinyl, pyridazinyl, pyrazinyl, triazinyl, quinolinyl, isoquinolinyl, quinoxalinyl, quinazolinyl, cinnolinyl, or naphthyridinyl.

42. The compound of any one of claims 1 to 41, wherein R³ is imidazolyl, triazolyl, indolyl, indazolyl, thiazolyl, isothiazolyl, or pyridinyl.

43. The compound of any one of claims 1 to 42, wherein R³ is selected from: 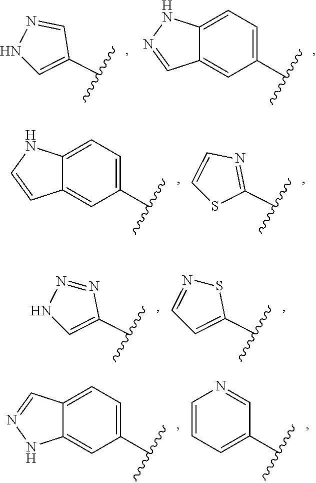
wherein R³ is substituted with 0 to 3 R¹².

44. The compound of any one of claims 1 to 40, wherein R³ is selected from:

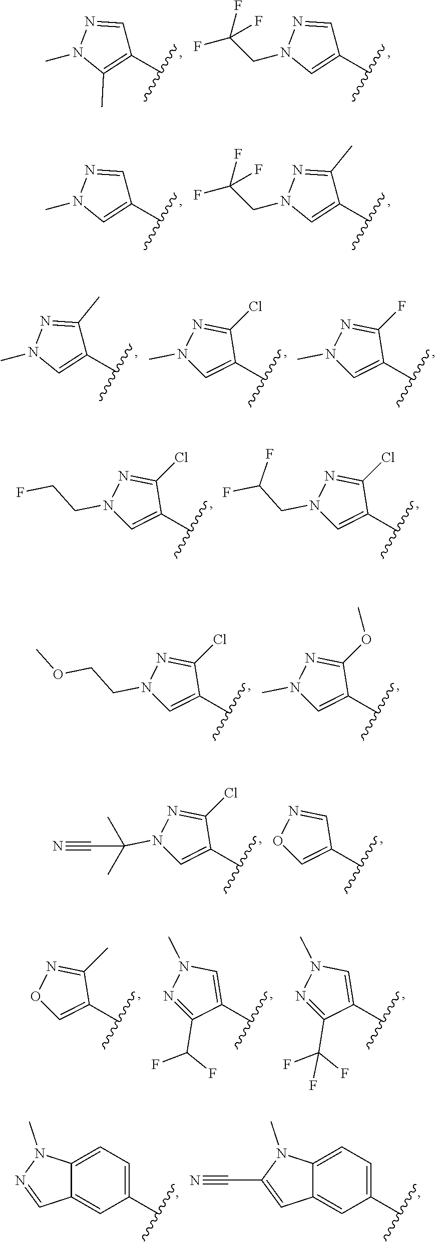

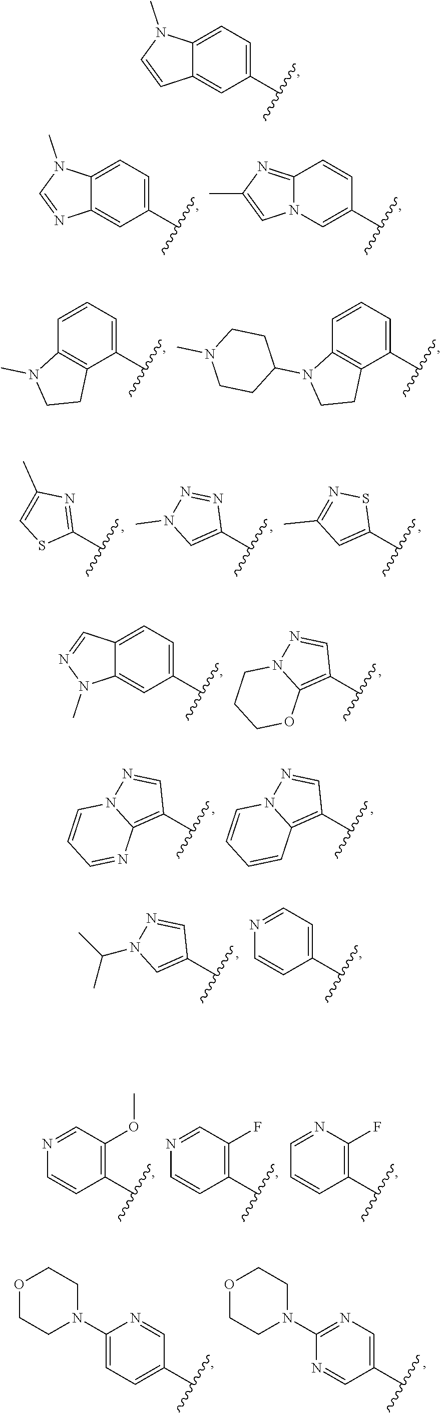

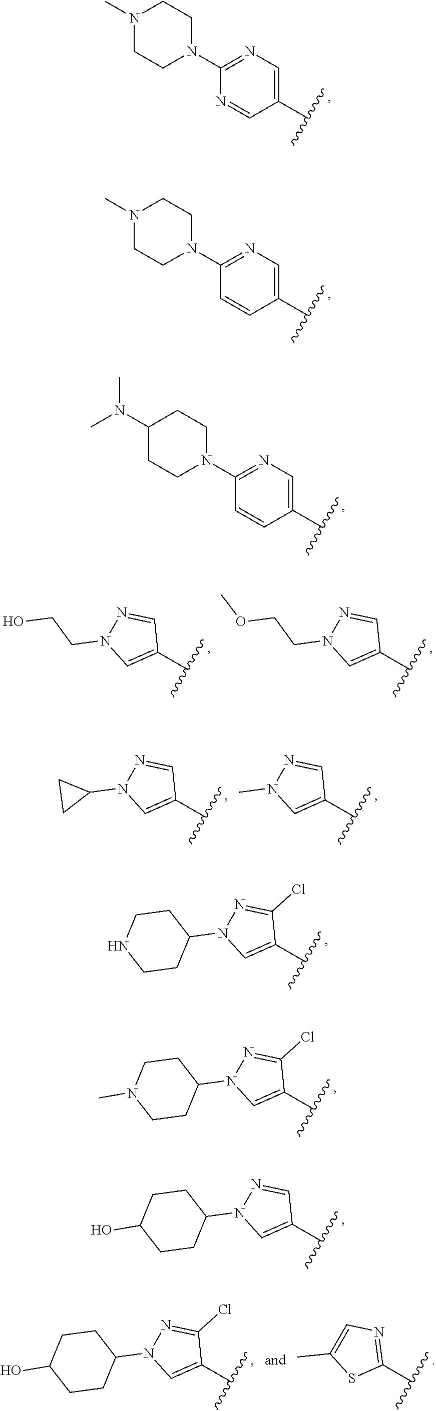

45. The compound of any one of claims 1 to 40 or 44, wherein R³ is selected from:

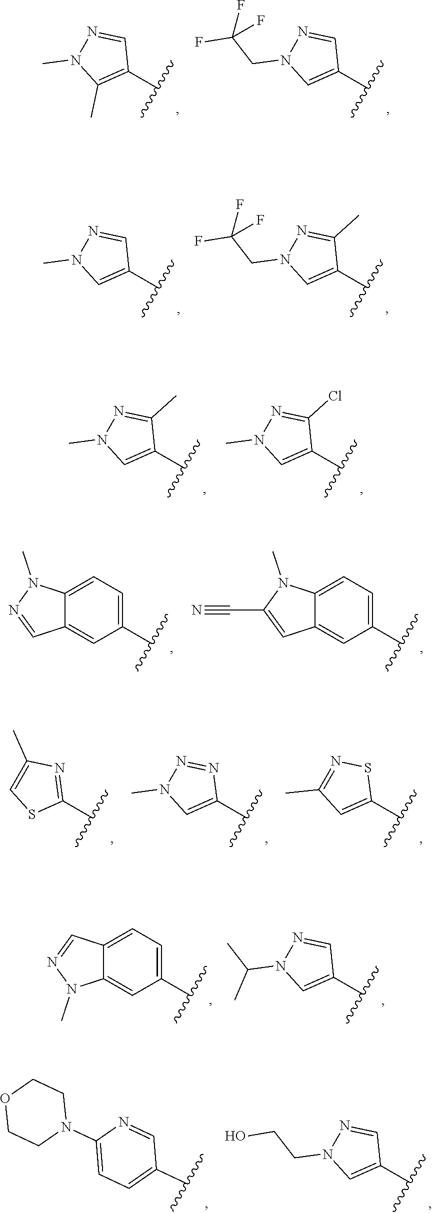

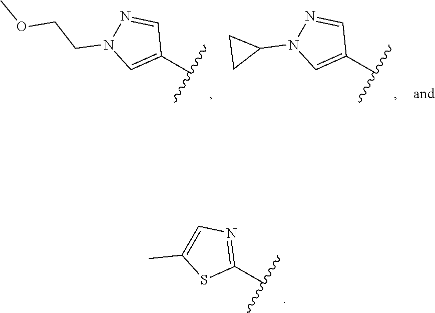

46. The compound of any one of claims 1 to 43, wherein R³ is unsubstituted.

47. The compound of any one of claims 1 to 43, wherein R³ is substituted with at least 1 R¹².

48. The compound of any one of claims 1 to 43 or 47, wherein R³ is substituted with at least 2 R¹².

49. The compound of any one of claims 1 to 43, 47, or 48, wherein each R¹² is independently aryl, heteroaryl, alkyl, heteroalkyl, haloalkyl, halo, cyano, heterocycloalkyl, —N(R¹³)₂, —S(═O)₂NH₂, or cycloalkyl.

50. The compound of any one of claims 1 to 43 or 47 to 49, wherein each R¹² is independently methyl, ethyl, n-propyl, iso-propyl, n-butyl, iso-butyl, sec-butyl, tert-butyl, hydroxyethyl, methoxyethyl, trifluoromethyl, trifluoroethyl, pentafluoroethyl, fluoro, chloro, cyano, azetidinyl, oxetanyl, pyrrolidinyl, imidazolidinyl, tetrahydrofuranyl, piperidinyl, piperazinyl, tetrahydropyranyl, morpholinyl, —N(R¹³)₂, cyclopropyl, cyclobutyl, cyclopentyl, or cyclohexyl.

51. The compound of any one of claims 1 to 43 or 47 to 50, wherein each R¹² is independently methyl, iso-propyl, tert-butyl, hydroxyethyl, methoxyethyl, trifluoromethyl, trifluoroethyl, chloro, cyano, morpholinyl, or cyclopropyl.

52. The compound of any one of claims 1 to 43 or 47 to 51, wherein each R¹² is independently methyl, hydroxyethyl, methoxyethyl, trifluoroethyl, or chloro.

53. The compound of any one of claims 1 to 43 or 47 to 52, wherein each R¹² is independently methyl or chloro.

54. The compound of any one of claims 1 to 43 or 47 to 50, wherein each R¹³ is independently alkyl or cycloalkyl.

55. The compound of any one of claims 1 to 43, 47 to 50, or 54, wherein each R¹³ is independently methyl, ethyl, n-propyl, iso-propyl, n-butyl, iso-butyl, sec-butyl, tert-butyl, cyclopropyl, cyclobutyl, cyclopentyl, or cyclohexyl.

56. The compound of any one of claims 1 to 43, 47 to 50, 54, or 55, wherein each R¹³ is independently methyl, ethyl, iso-propyl, tert-butyl, cyclopropyl, cyclopentyl, or cyclohexyl.

57. The compound of any one of claims 1 to 43, 47 to 50, or 54 to 56, wherein each R¹³ is independently methyl, cyclopropyl, or cyclohexyl.

58. The compound of any one of claims 1 to 49, wherein the aryl, heteroaryl, heterocycloalkyl, or cycloalkyl of R¹² is unsubstituted.

59. The compound of any one of claims 1 to 49, wherein the aryl, heteroaryl, heterocycloalkyl, or cycloalkyl of R¹² is substituted with 1 or 2 R¹⁴.

60. The compound of any one of claims 1 to 49 or 59, wherein each R¹⁴ is independently alkyl, cycloalkyl, heterocycloalkyl, halo, cyano, —N(R¹⁵)₂, or alkoxy.

61. The compound of any one of claims 1 to 49, 59, or 60, wherein each R¹⁴ is independently methyl, ethyl, n-propyl, iso-propyl, n-butyl, iso-butyl, sec-butyl, tert-butyl, cyclopropyl, cyclobutyl, cyclopentyl, cyclohexyl, azetidinyl, oxetanyl, pyrrolidinyl, imidazolidinyl, tetrahydrofuranyl, piperidinyl, piperazinyl, tetrahydropyranyl, morpholinyl, fluoro, chloro, cyano, —N(R¹⁵)₂, methoxy, ethoxy, or trifluoromethoxy.

62. The compound of any one of claims 1 to 49, or 59 to 61, wherein each R¹⁴ is independently methyl, ethyl, iso-propyl, tert-butyl, pyrrolidinyl, piperidinyl, morpholinyl, fluoro, chloro, —N(R¹⁵)₂, or methoxy.

63. The compound of any one of claims 1 to 49 or 59 to 62, wherein each R¹⁵ is independently alkyl or cycloalkyl.

64. The compound of any one of claims 1 to 49 or 59 to 63, wherein each R¹⁵ is methyl, ethyl, n-propyl, iso-propyl, n-butyl, iso-butyl, sec-butyl, tert-butyl, cyclopropyl, cyclobutyl, cyclopentyl, or cyclohexyl.

65. The compound of any one of claims 1 to 49 or 59 to 64, wherein each R¹³ is independently methyl, ethyl, iso-propyl, tert-butyl, cyclopropyl, cyclopentyl, or cyclohexyl.

66. The compound of any one of claims 1 to 49 or 59 to 65, wherein each R¹³ is independently methyl, cyclopropyl, or cyclohexyl.

67. The compound of claim 1, wherein the compound is selected from:

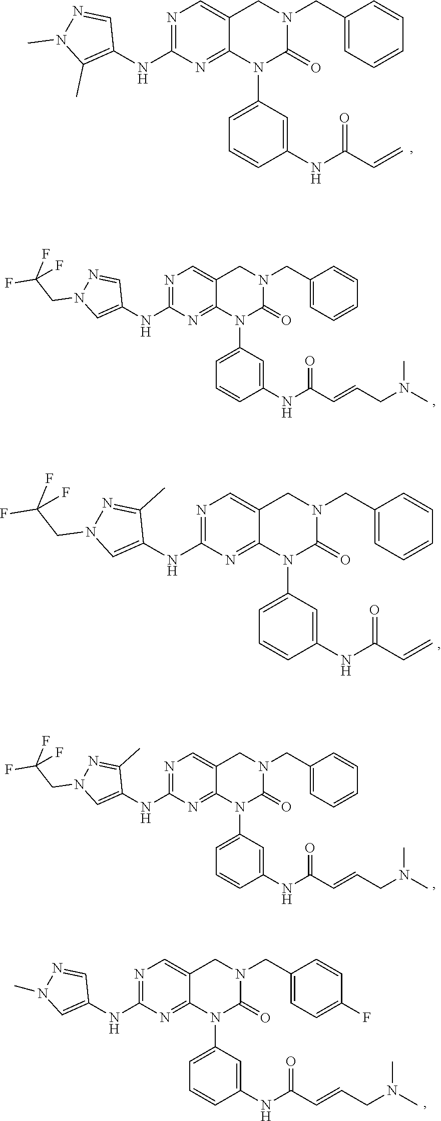

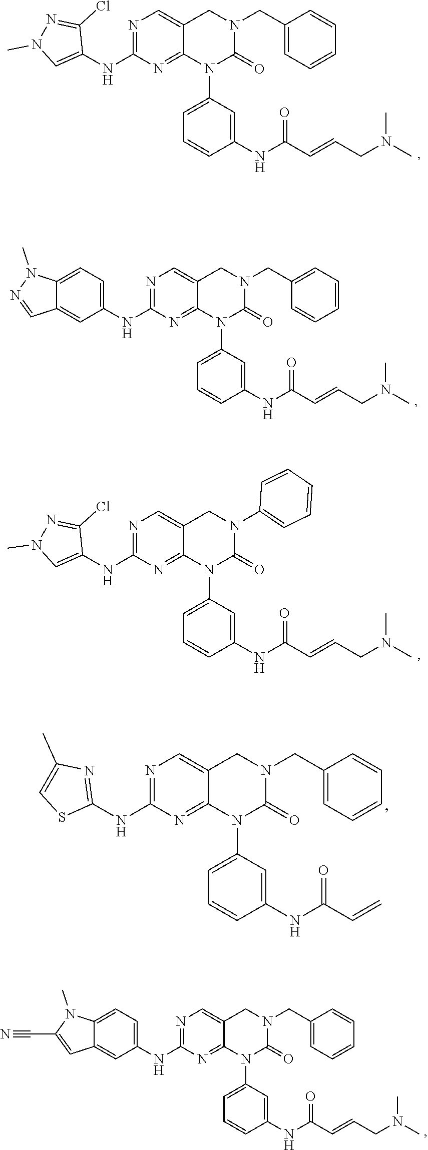

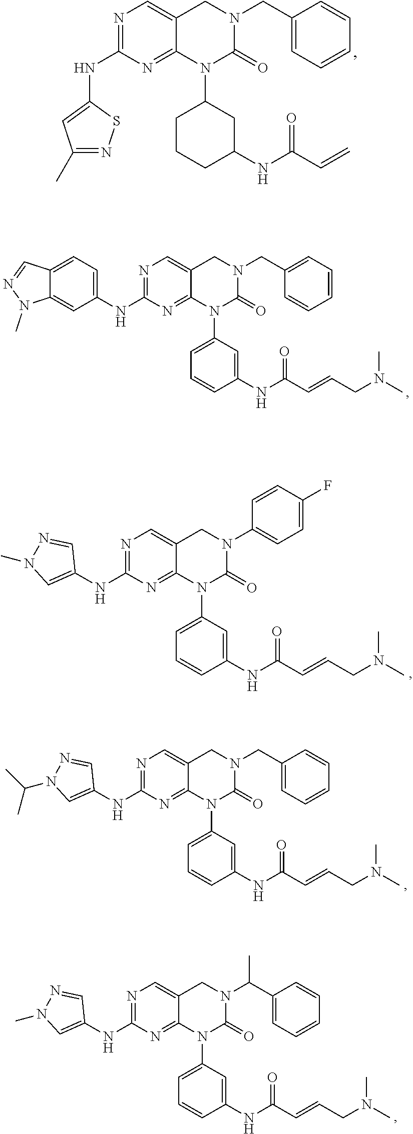

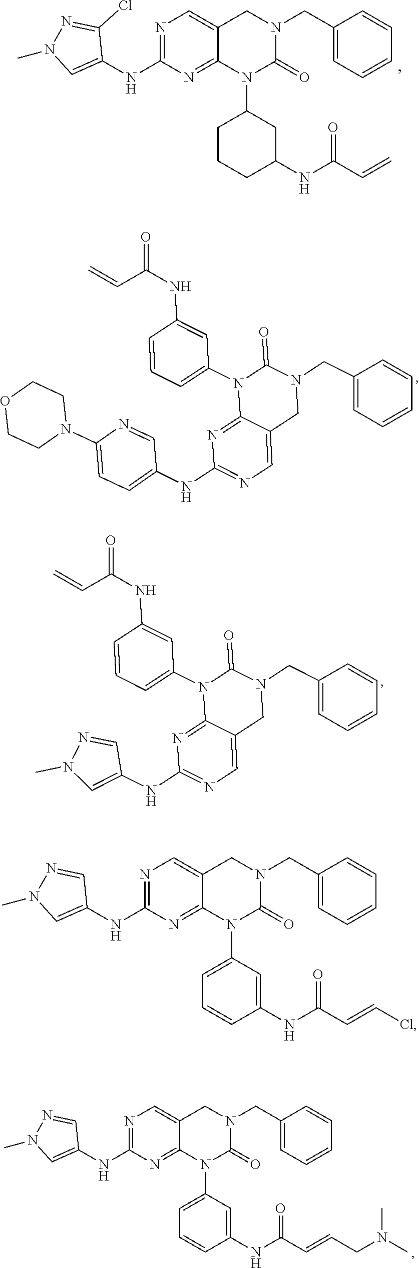

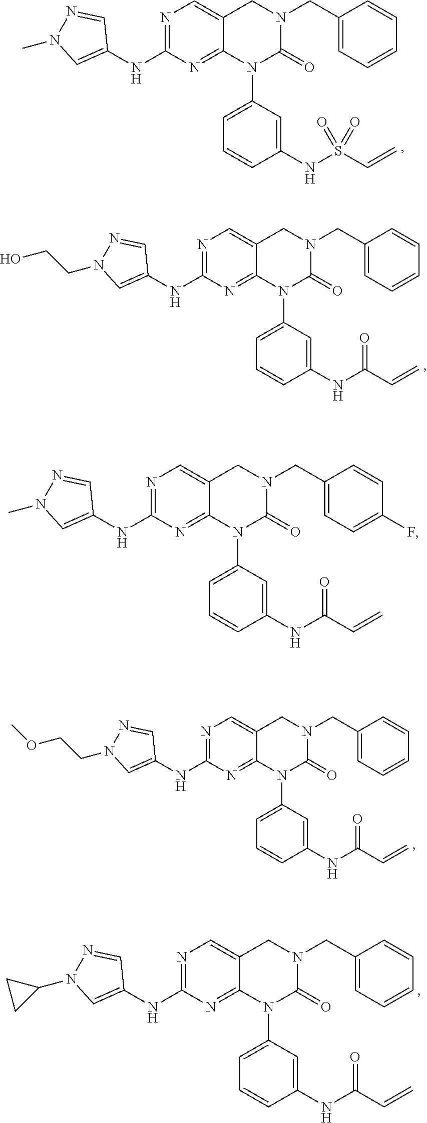 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; C=CC(=O)Nc1cccc(N2C(=O)N(Cc3ccccc3)Cc3cnc(Nc4cnn(C5CC5)c4)nc32)c1]

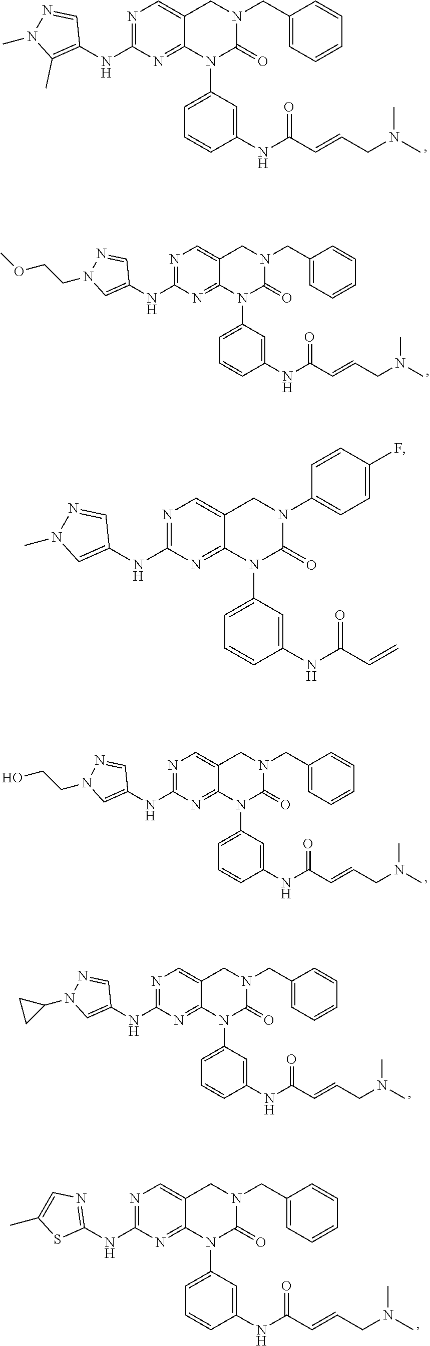

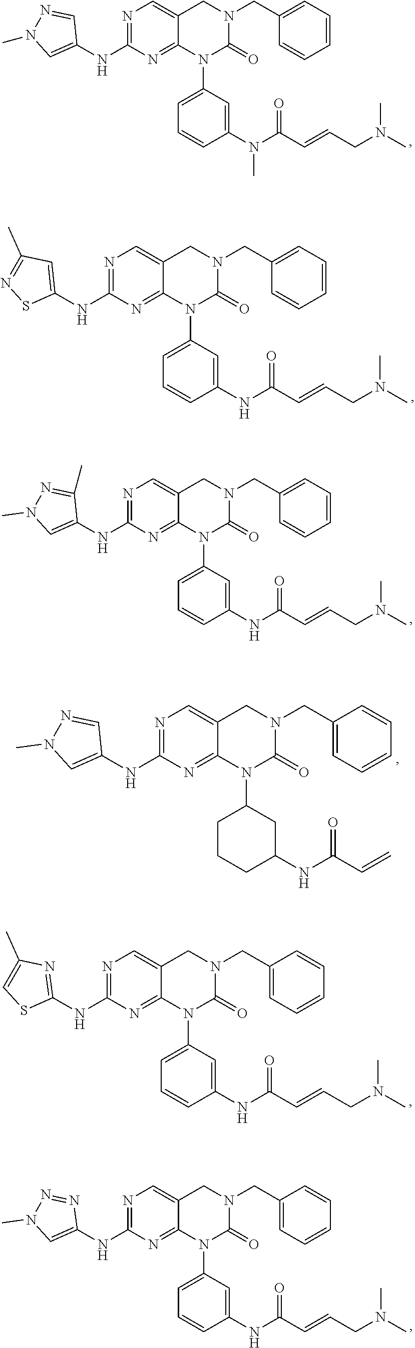

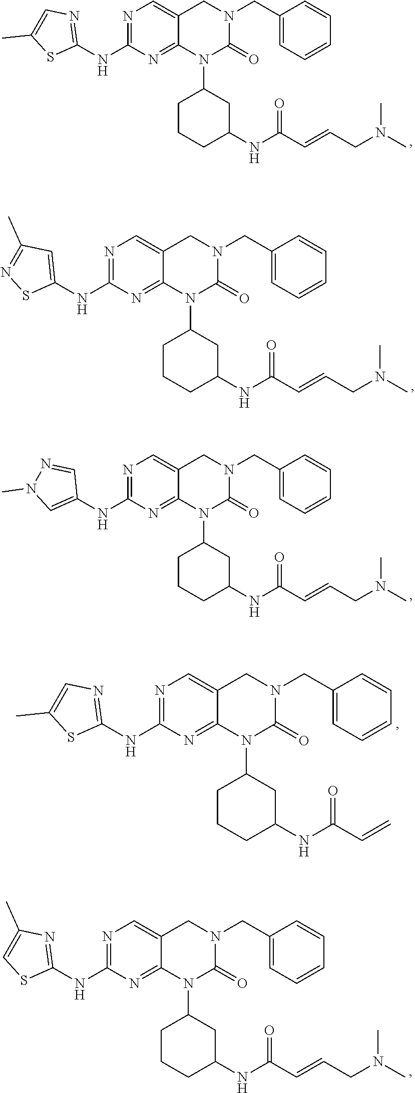

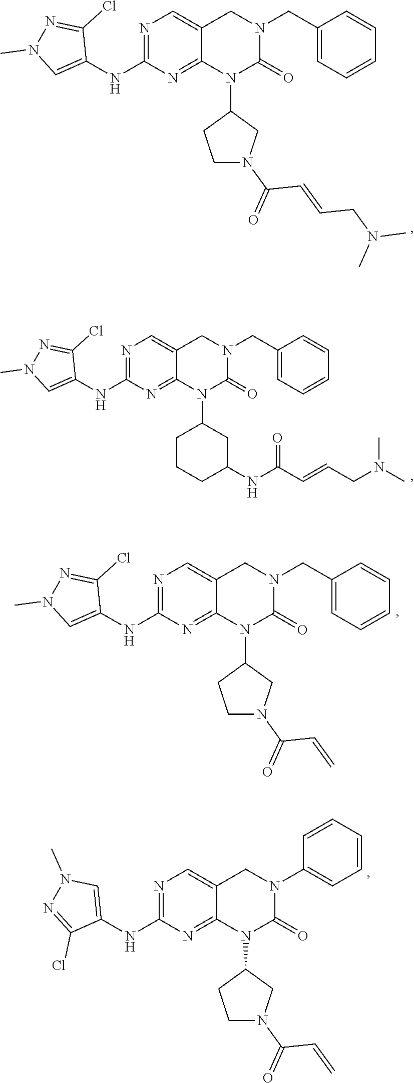

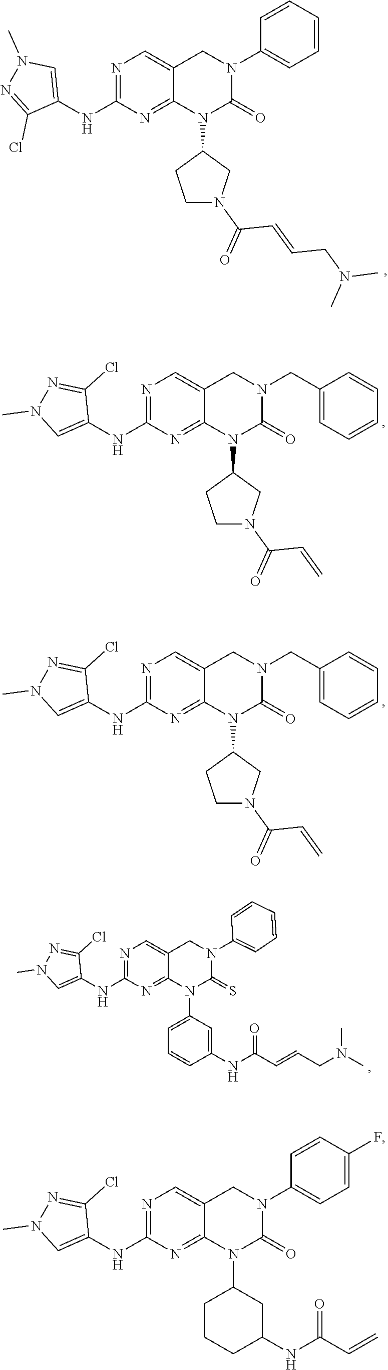

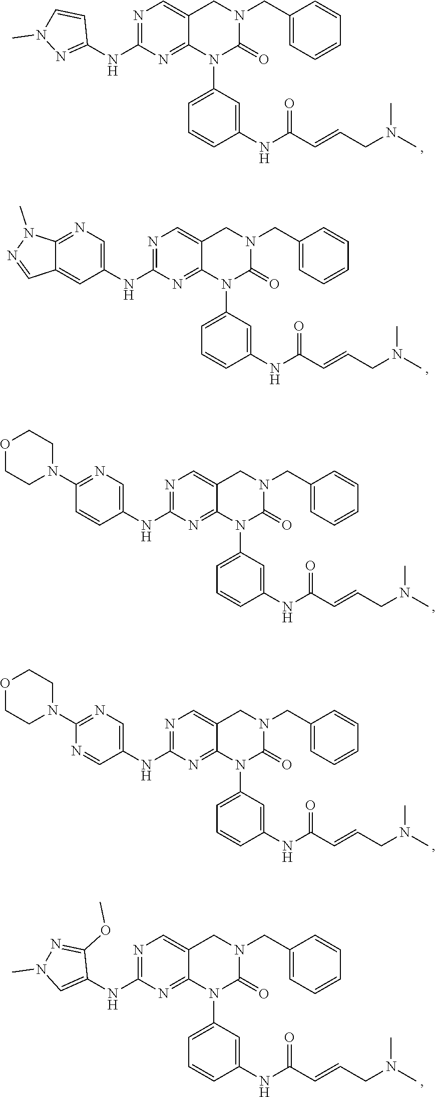

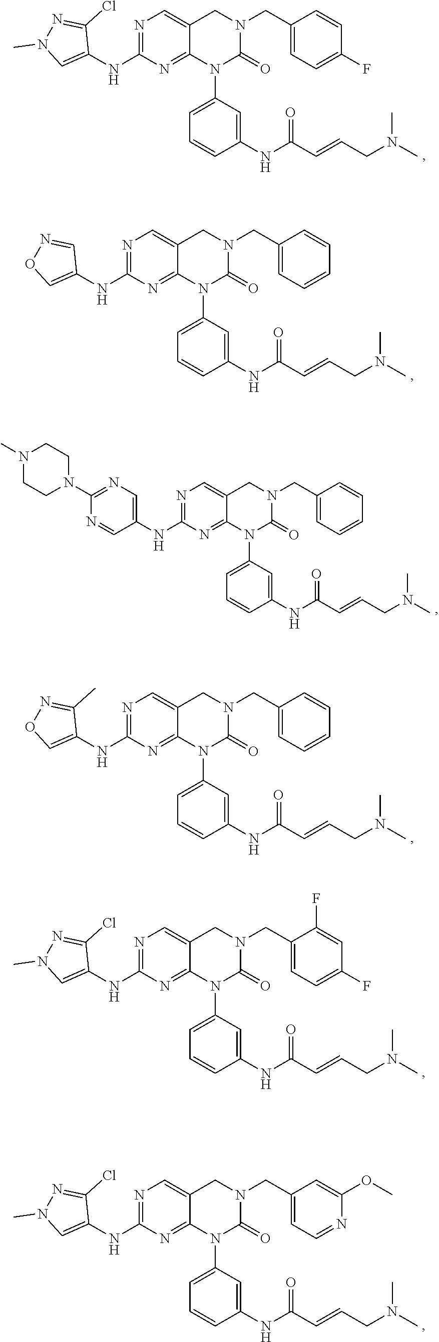

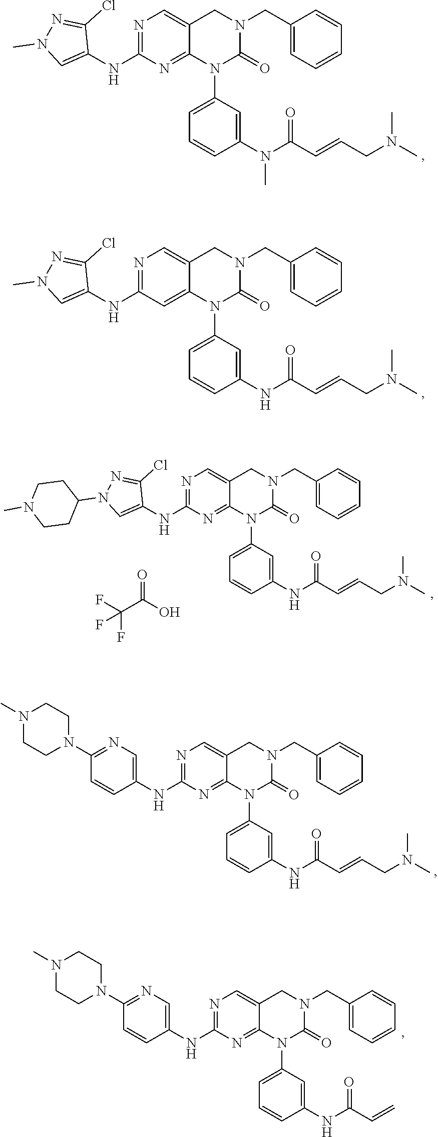

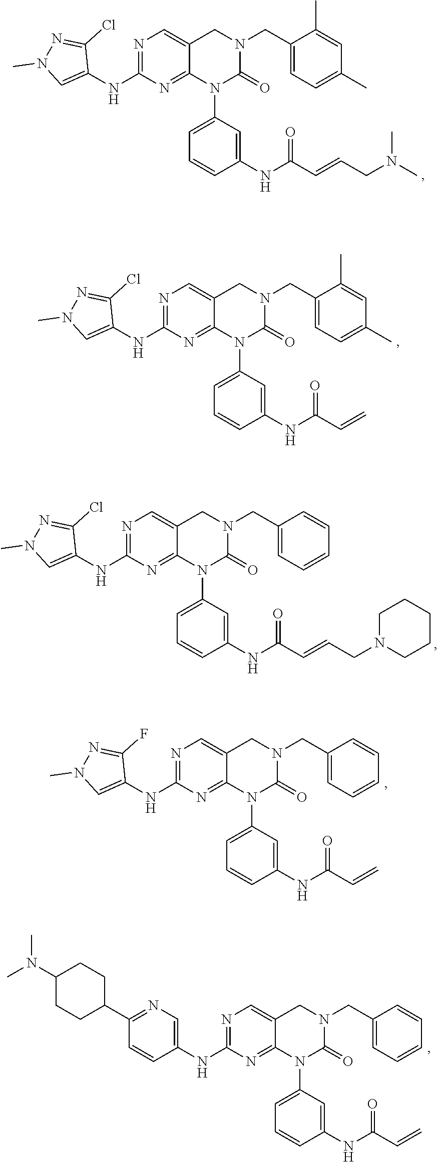

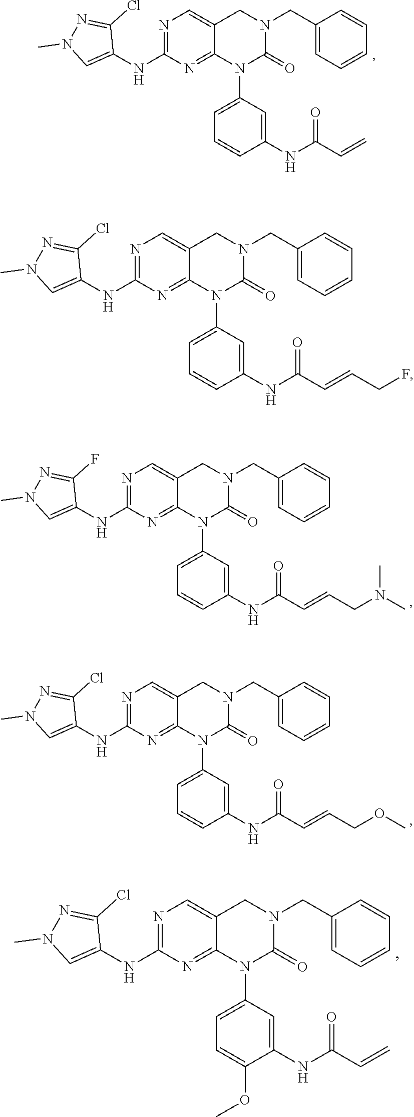

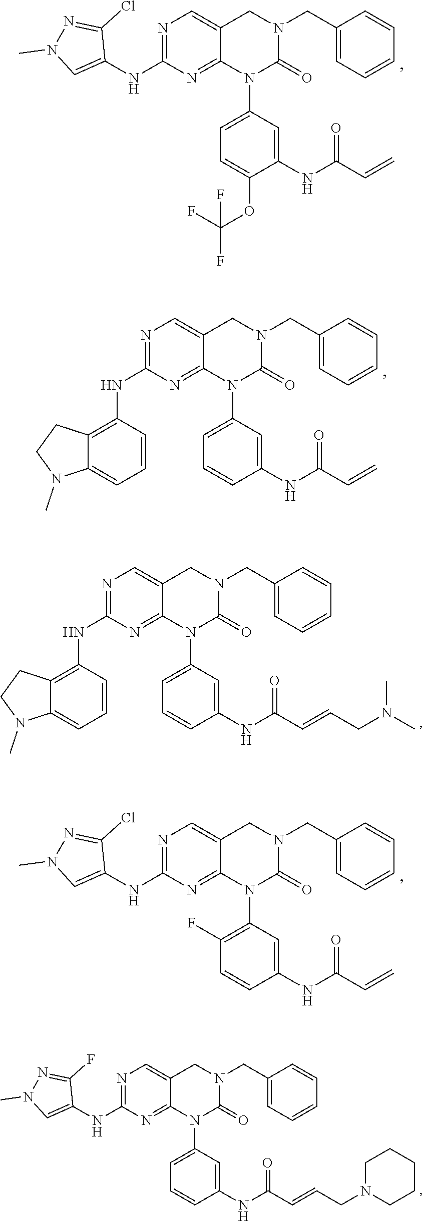

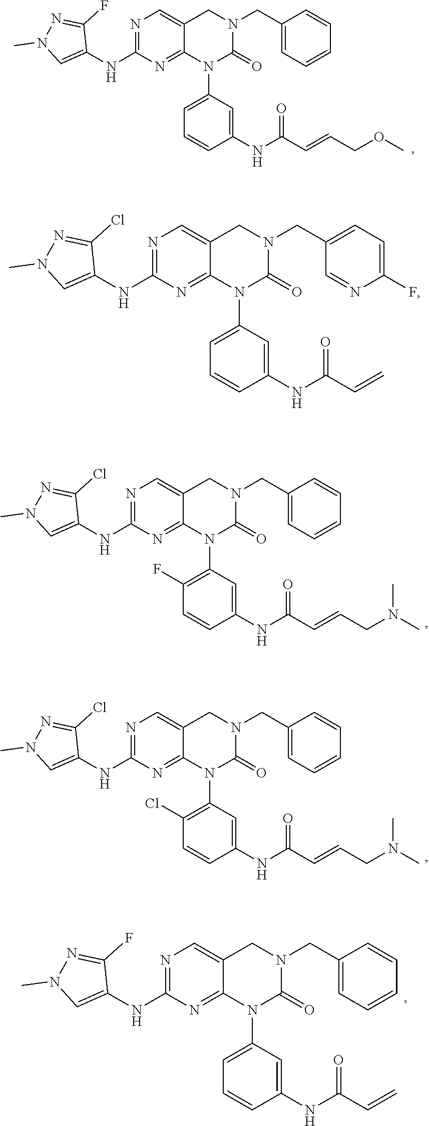

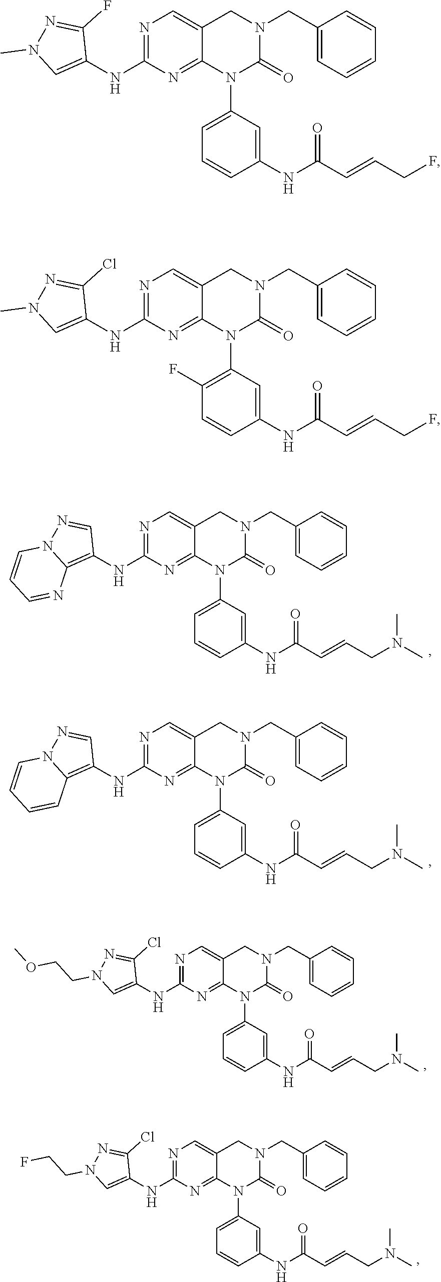

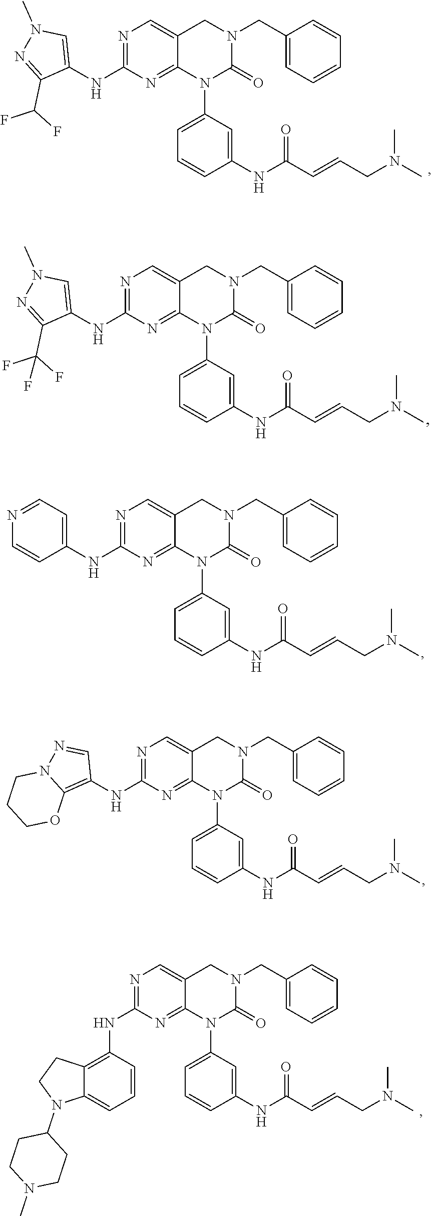 [SMILES: CN(C)C/C=C/C(=O)Nc1cccc(N2C(=O)N(Cc3ccccc3)Cc3cnc(Nc4cn(C)nc4C(F)F)nc32)c1 ; CN(C)C/C=C/C(=O)Nc1cccc(N2C(=O)N(Cc3ccccc3)Cc3cnc(Nc4cn(C)nc4C(F)(F)F)nc32)c1 ; CN(C)C/C=C/C(=O)Nc1cccc(N2C(=O)N(Cc3ccccc3)Cc3cnc(Nc4ccncc4)nc32)c1 ; CN(C)C/C=C/C(=O)Nc1cccc(N2C(=O)N(Cc3ccccc3)Cc3cnc(Nc4cnn5c4OCCC5)nc32)c1 ; CN(C)C/C=C/C(=O)Nc1cccc(N2C(=O)N(Cc3ccccc3)Cc3cnc(Nc4cccc5c4CCN5C4CCN(C)CC4)nc32)c1]

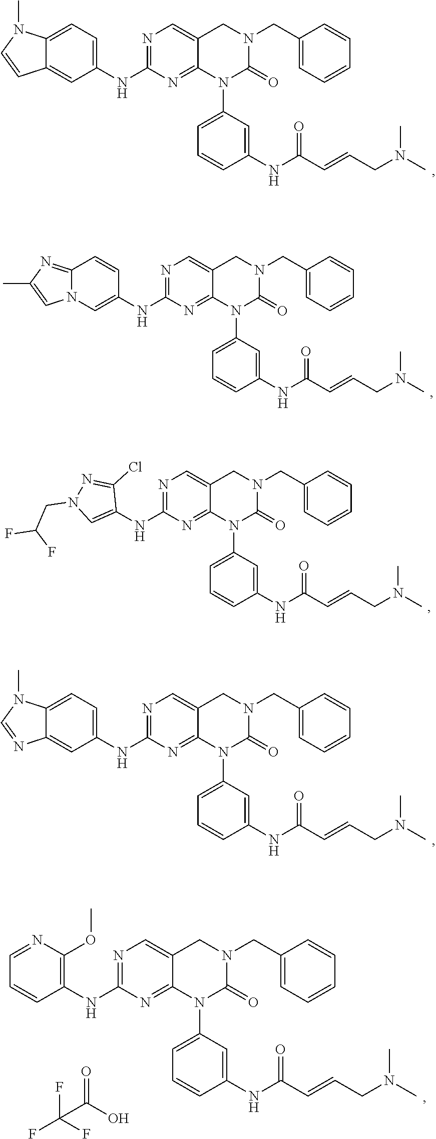

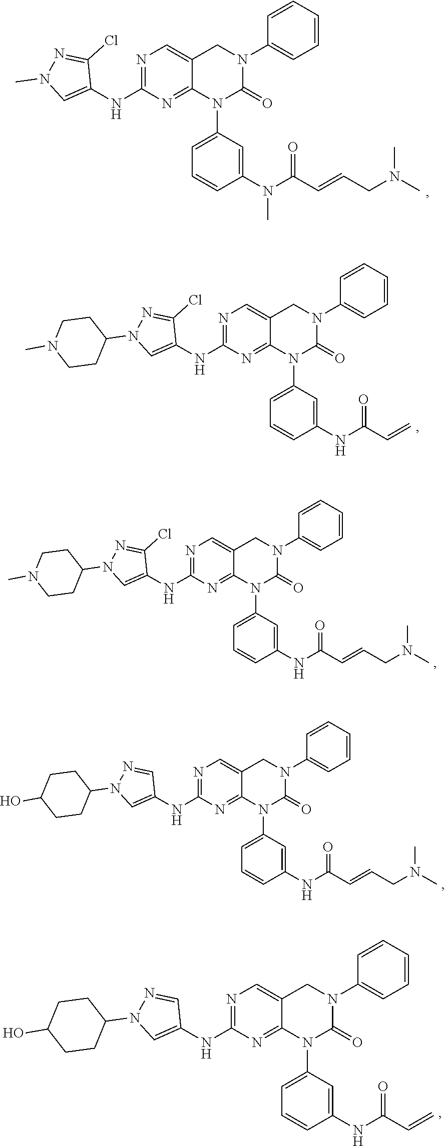

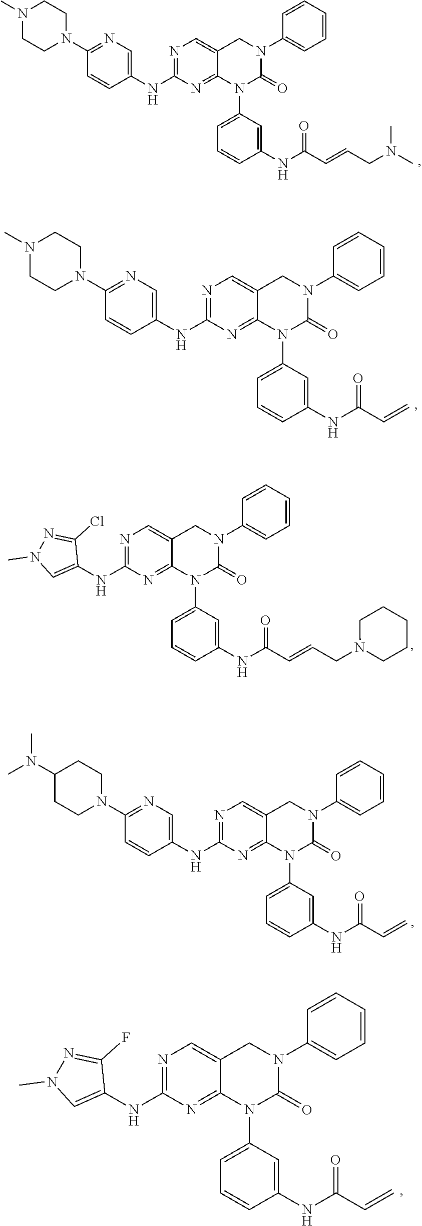

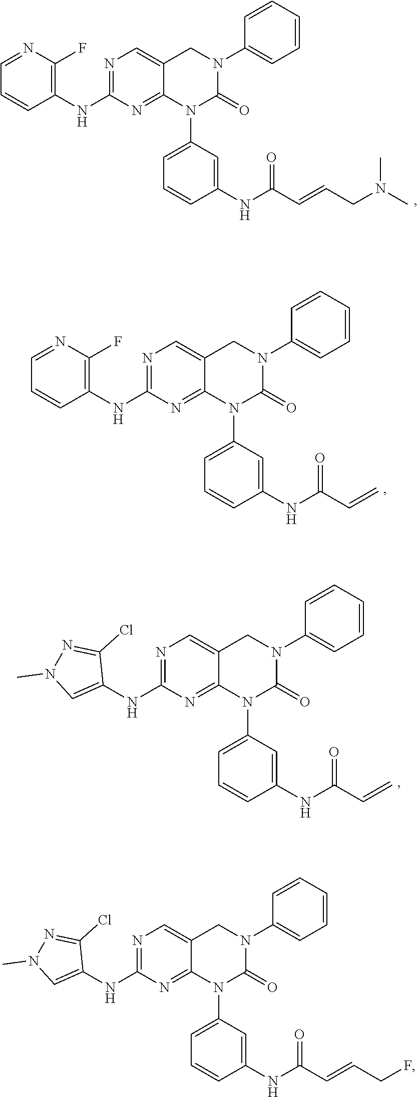

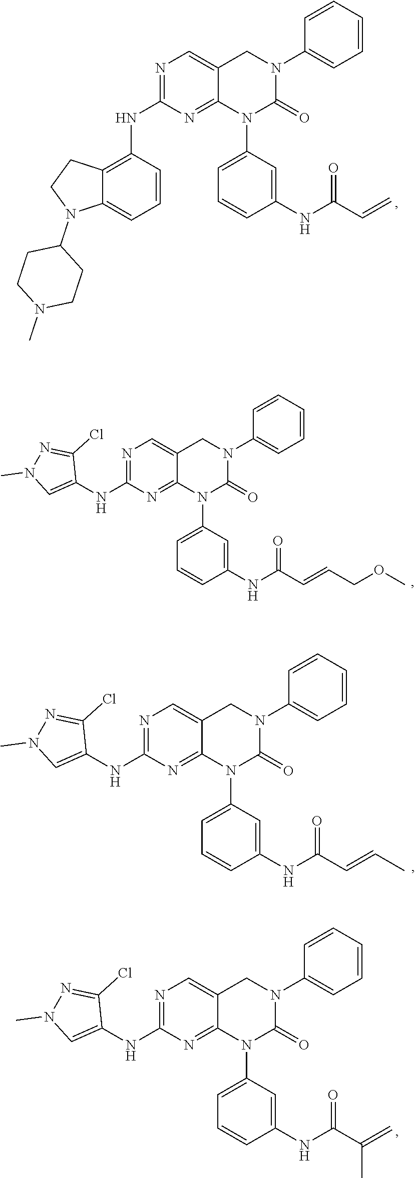

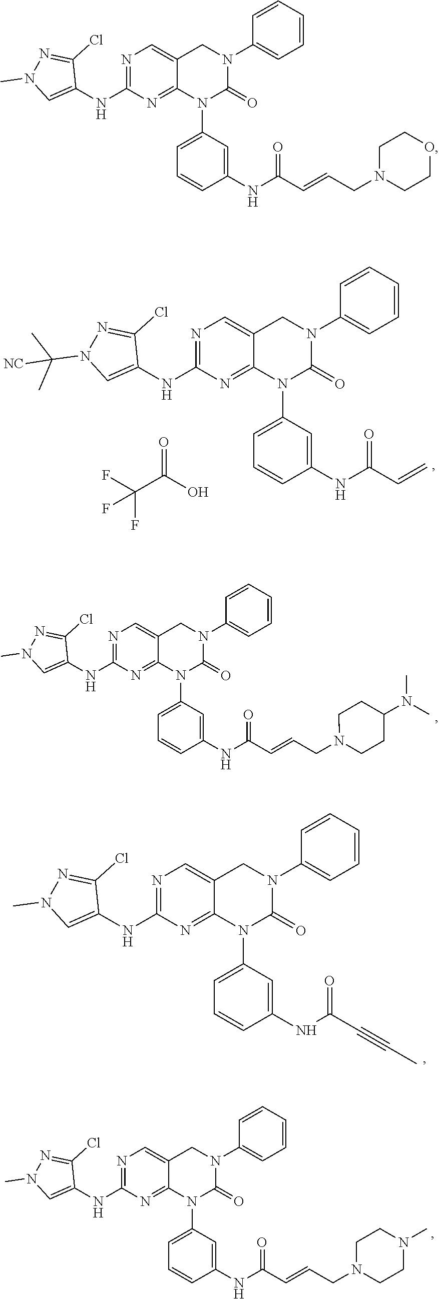

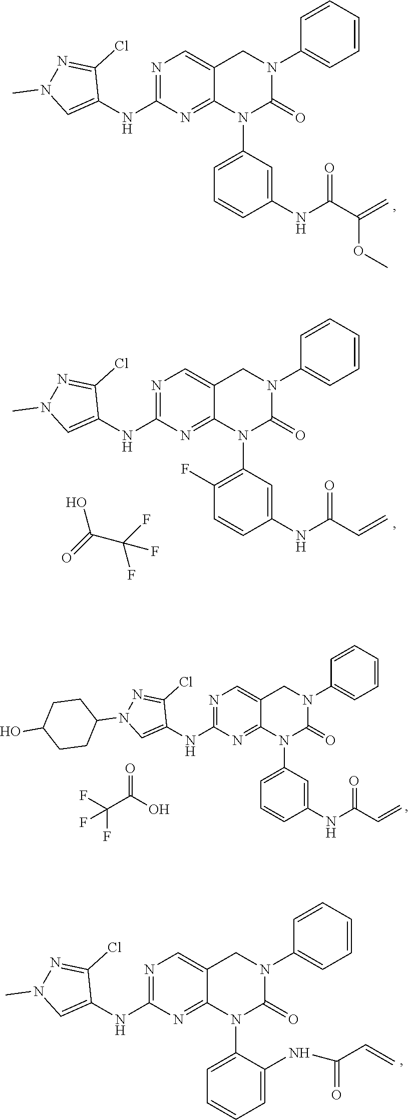

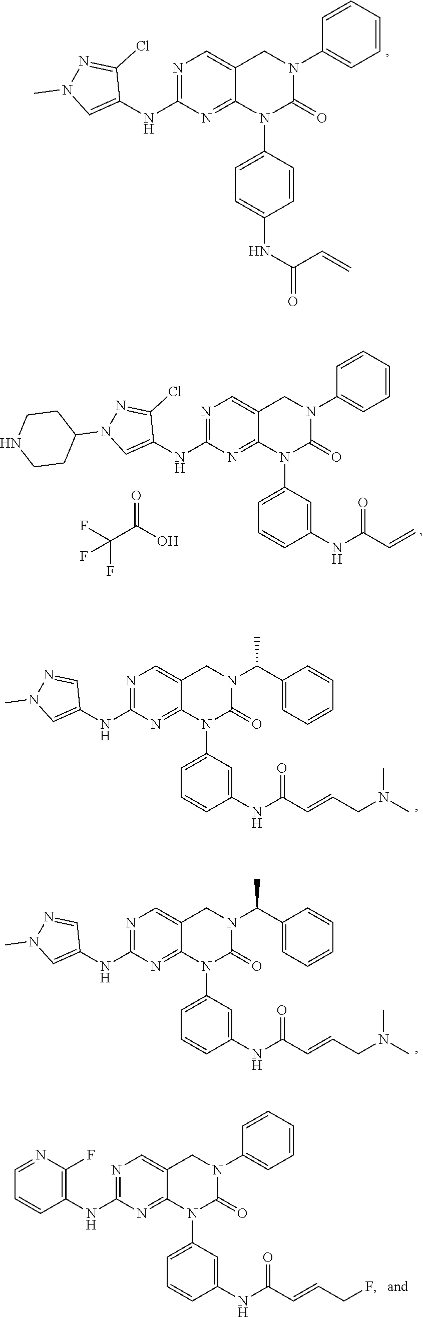

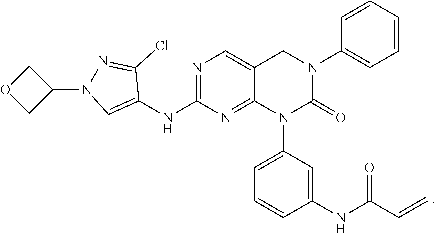 [SMILES: C=CC(=O)Nc1cccc(N2C(=O)N(c3ccccc3)Cc3cnc(Nc4cn(C5COC5)nc4Cl)nc32)c1]

68. The compound of claim 1, wherein the compound is selected from:

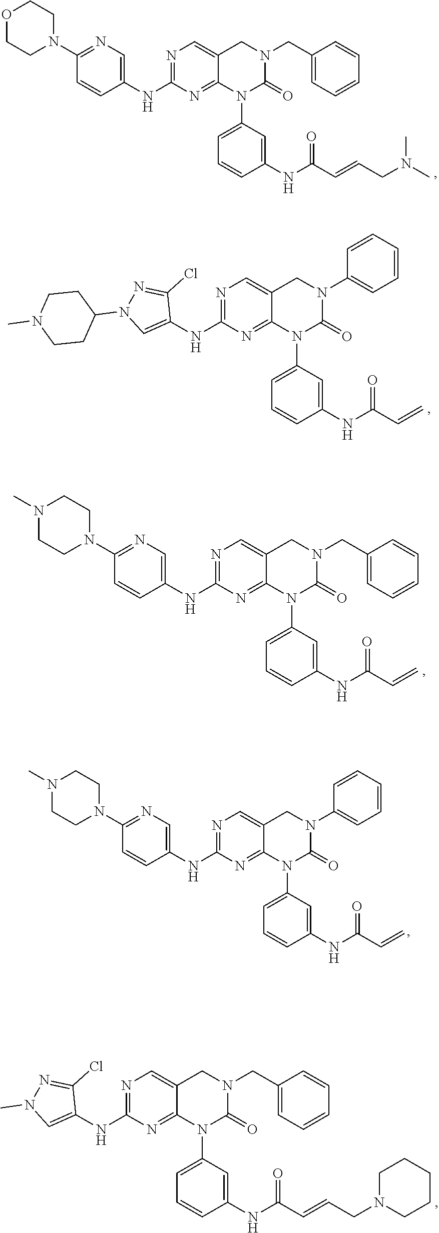

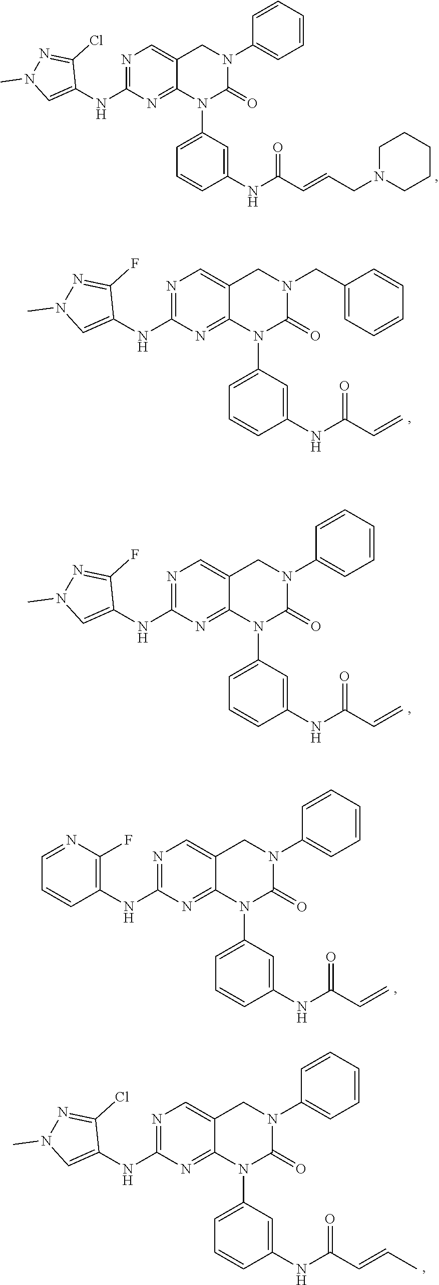

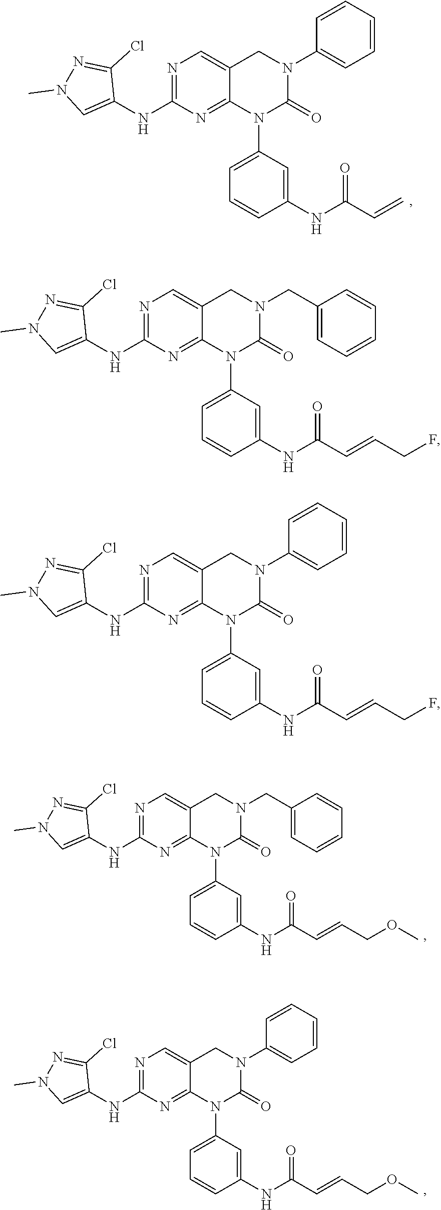

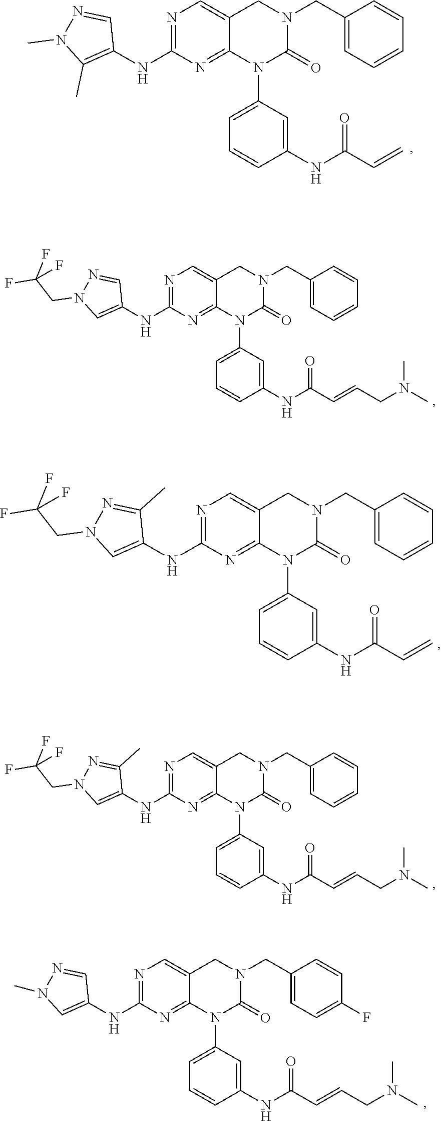

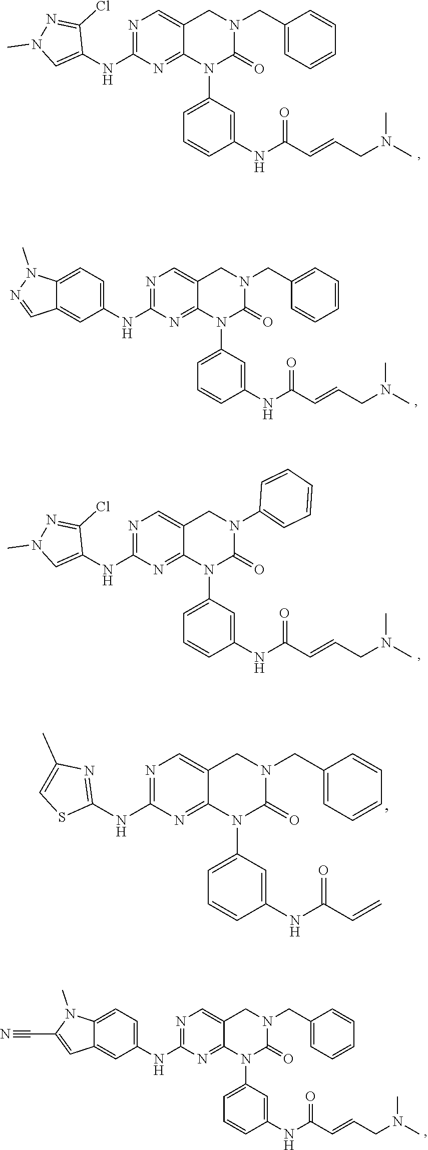

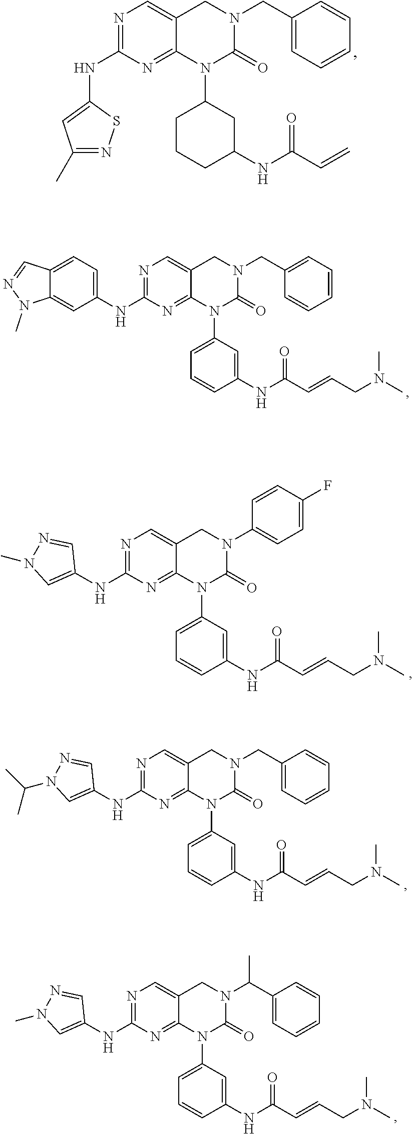

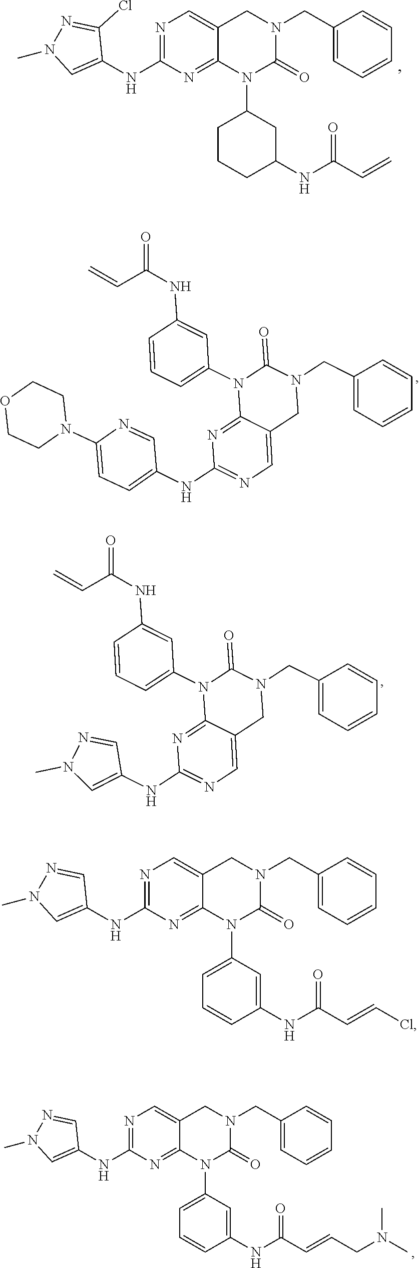

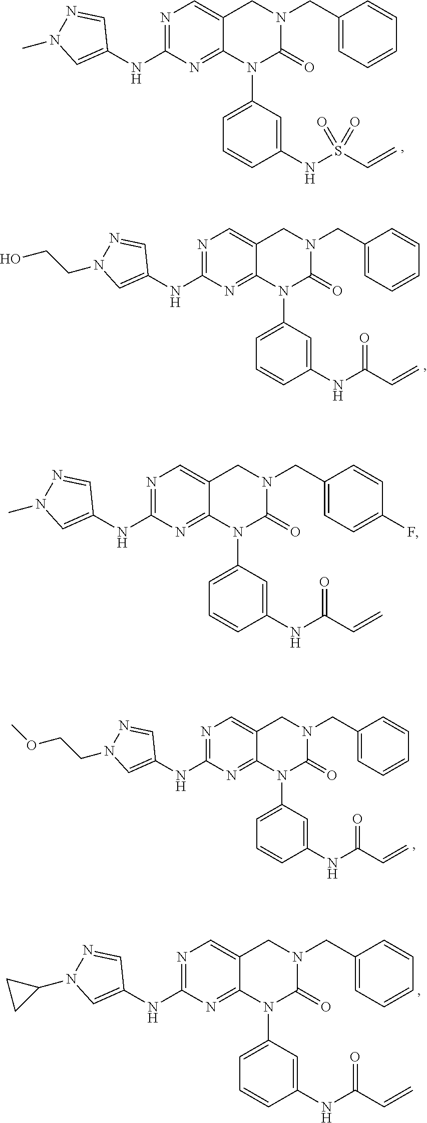 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; C=CC(=O)Nc1cccc(N2C(=O)N(Cc3ccccc3)Cc3cnc(Nc4cnn(C5CC5)c4)nc32)c1]

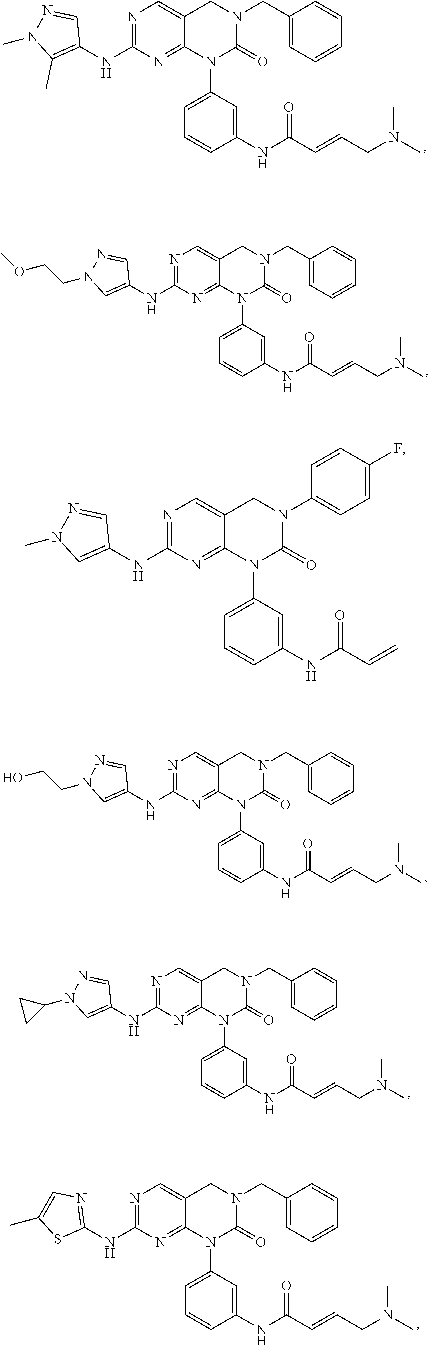

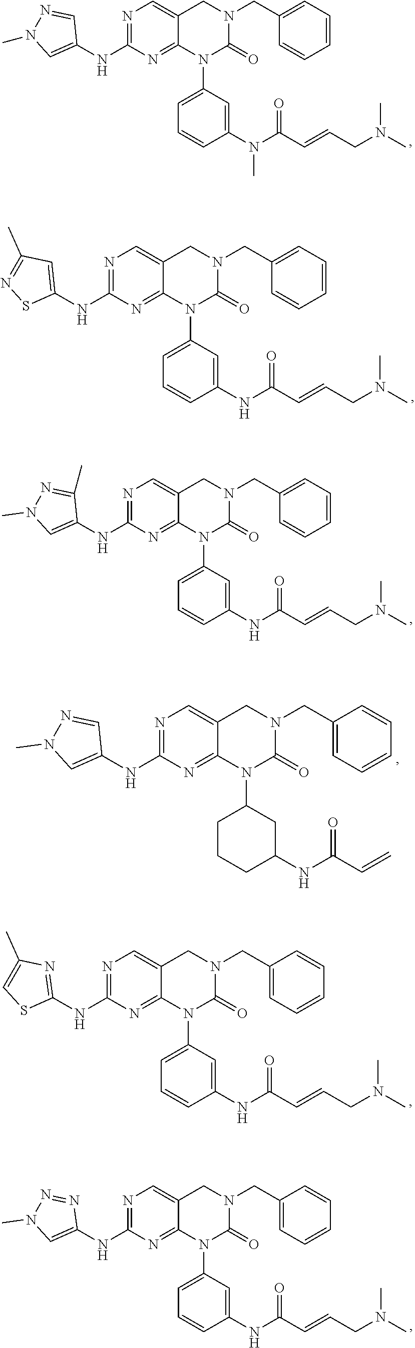

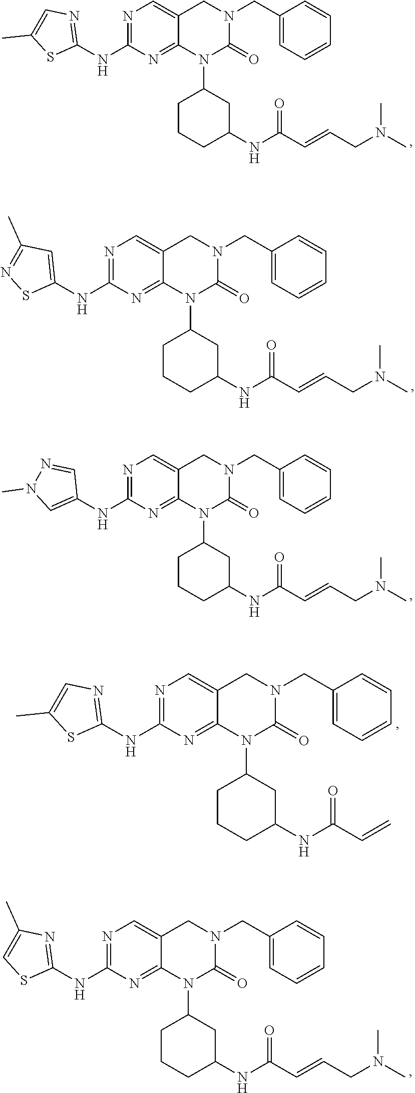

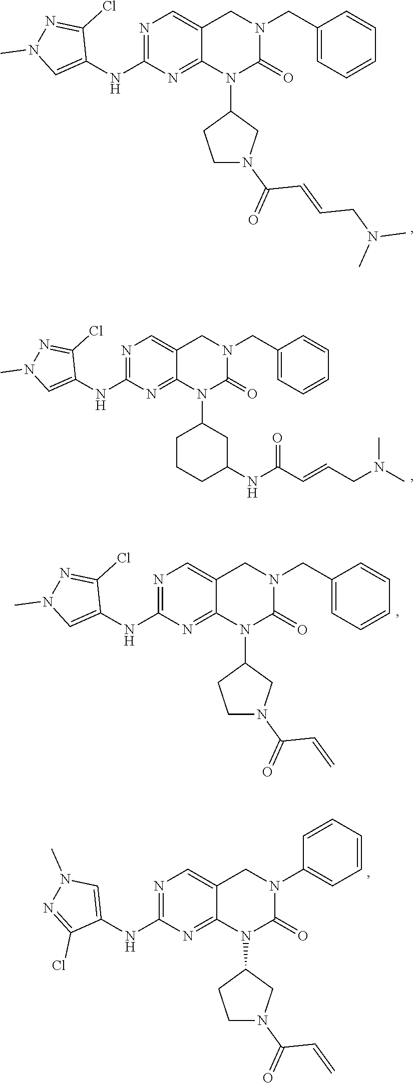

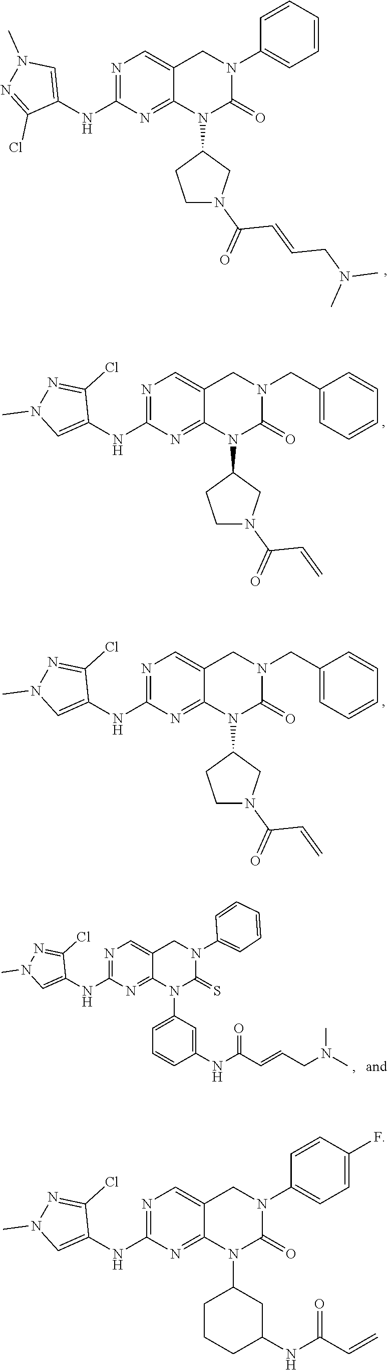

69. The compound of claim 1, wherein the compound is selected from:

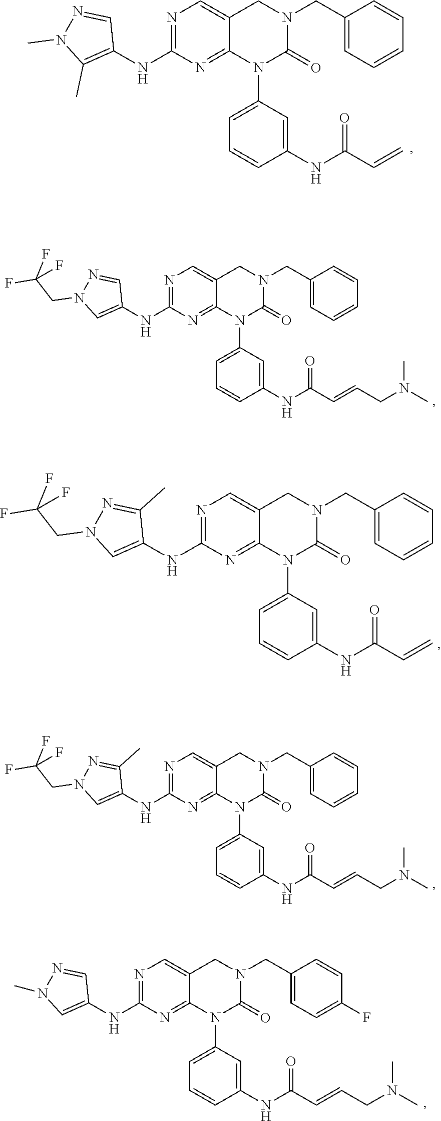

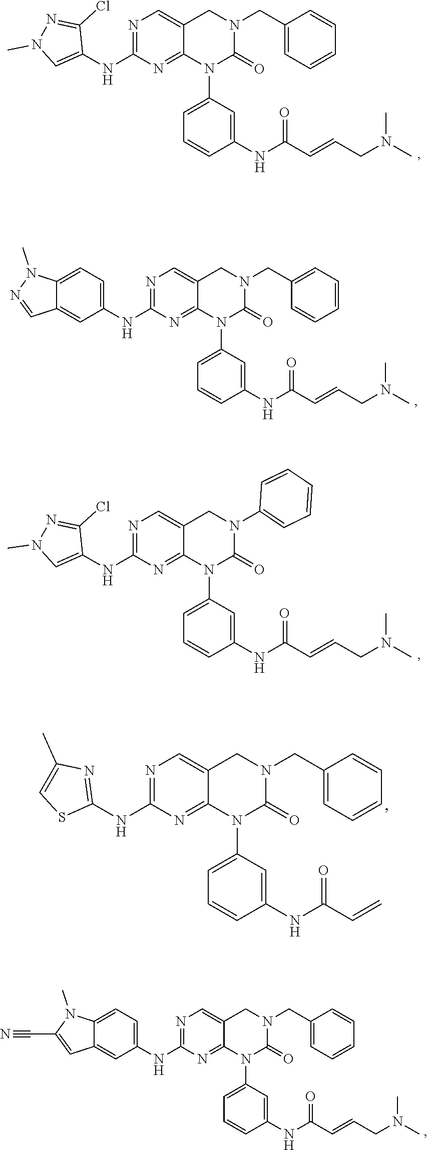

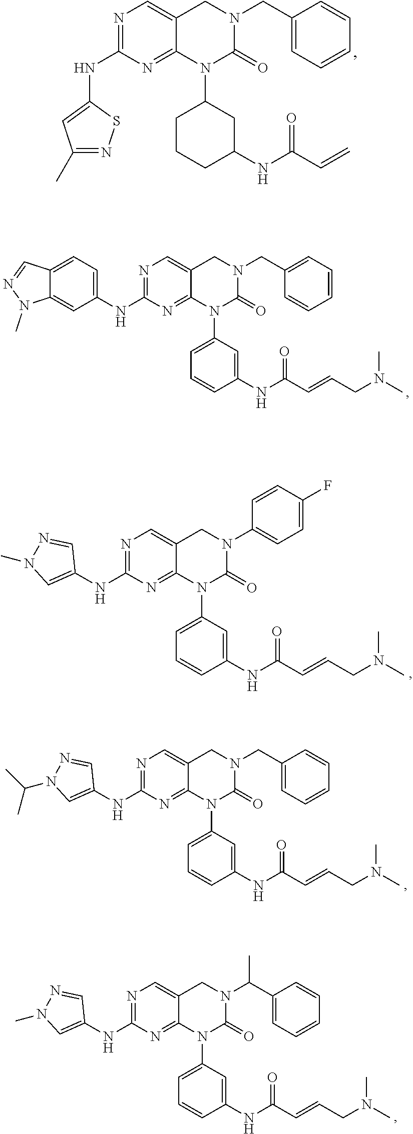

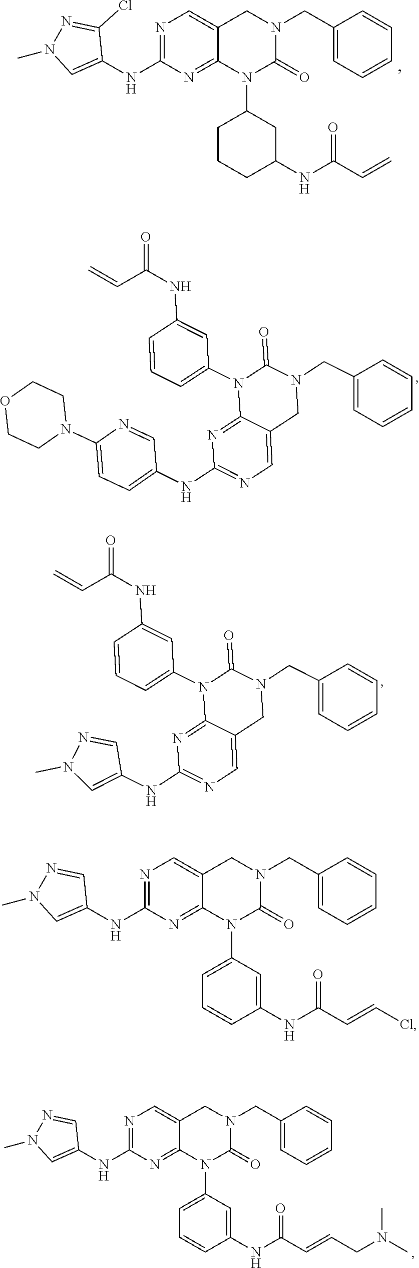

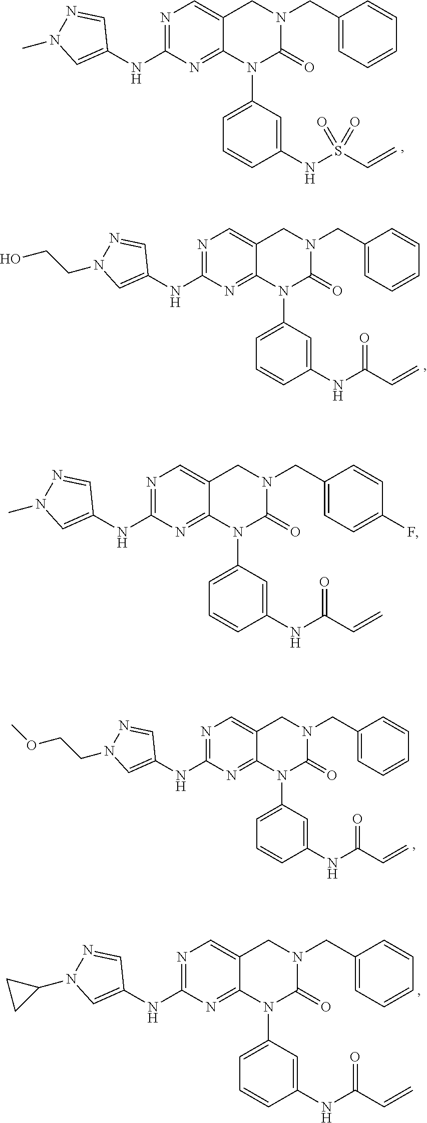 [SMILES: C=CS(=O)(=O)Nc1cccc(N2C(=O)N(Cc3ccccc3)Cc3cnc(Nc4cnn(C)c4)nc32)c1 ; C=CC(=O)Nc1cccc(N2C(=O)N(Cc3ccccc3)Cc3cnc(Nc4cnn(CCO)c4)nc32)c1 ; C=CC(=O)Nc1cccc(N2C(=O)N(Cc3ccc(F)cc3)Cc3cnc(Nc4cnn(C)c4)nc32)c1 ; C=CC(=O)Nc1cccc(N2C(=O)N(Cc3ccccc3)Cc3cnc(Nc4cnn(CCOC)c4)nc32)c1 ; C=CC(=O)Nc1cccc(N2C(=O)N(Cc3ccccc3)Cc3cnc(Nc4cnn(C5CC5)c4)nc32)c1]

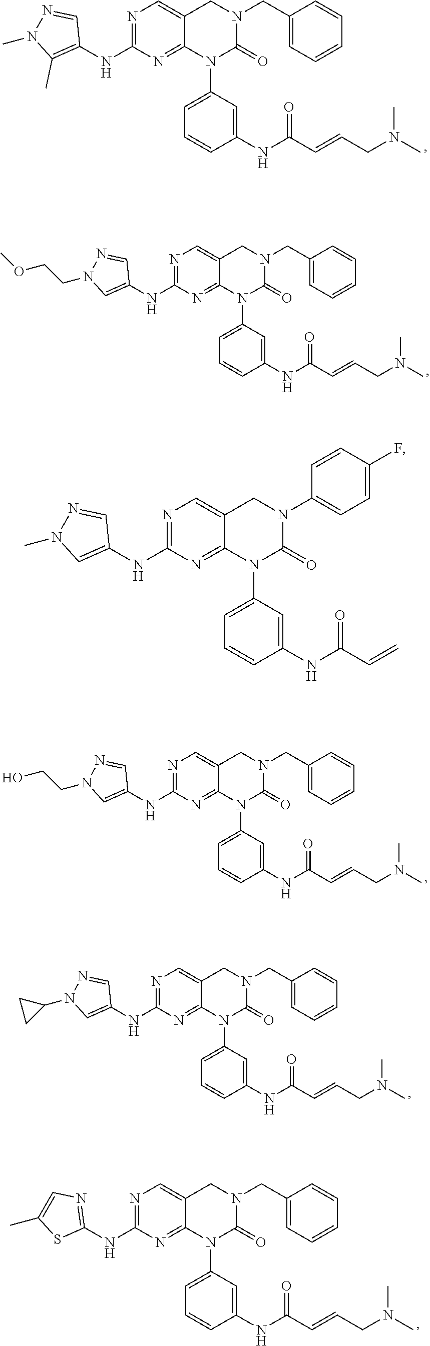

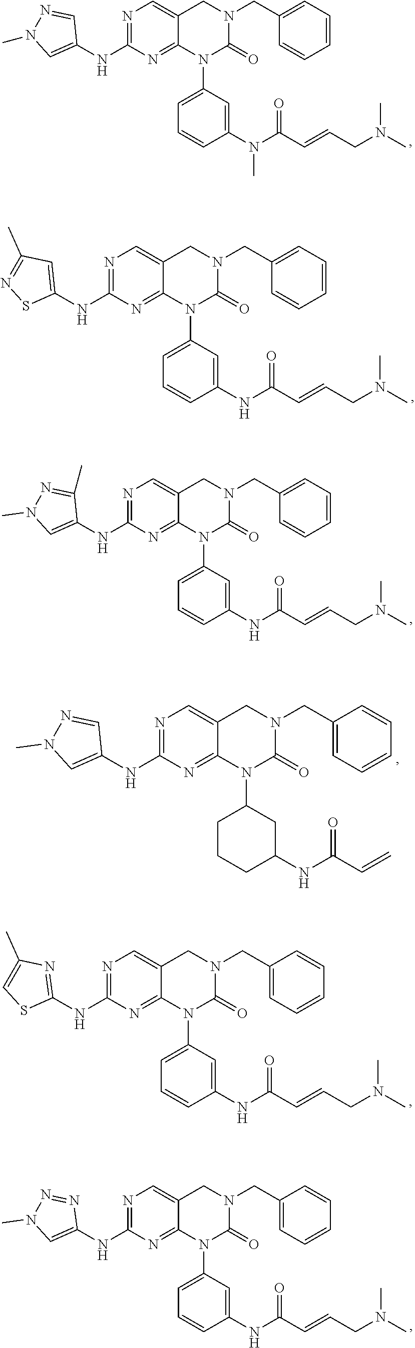

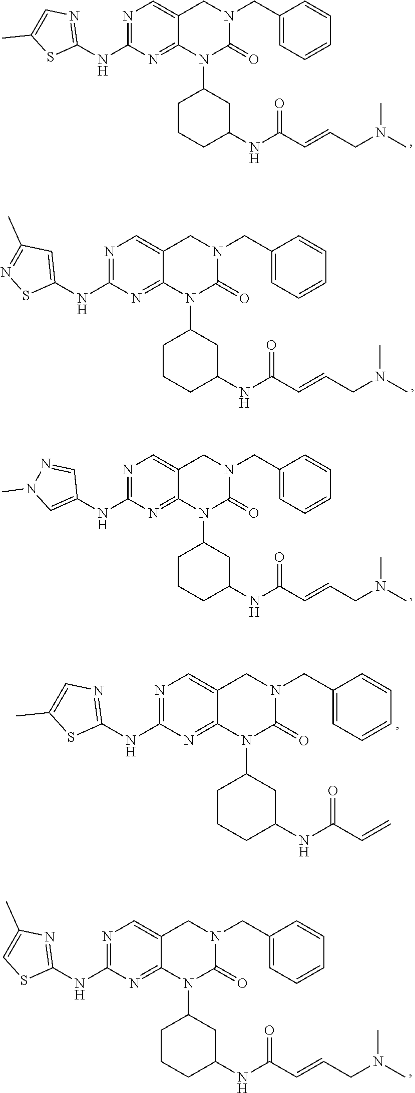

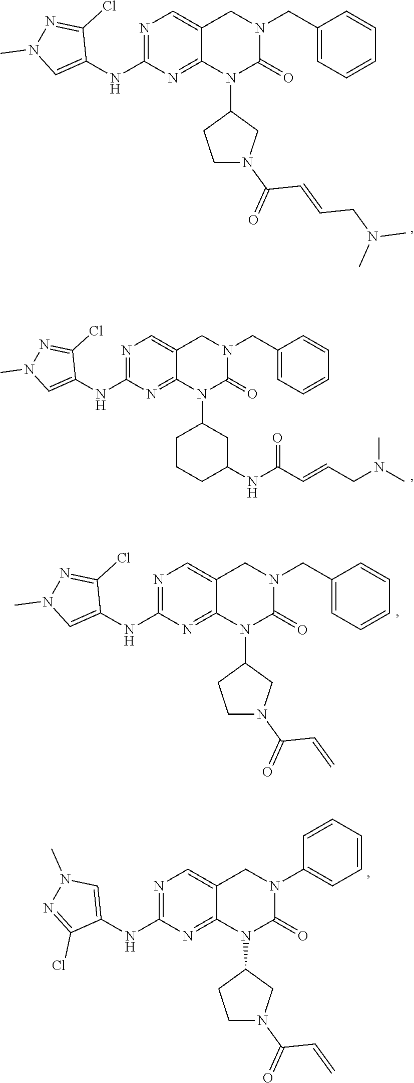

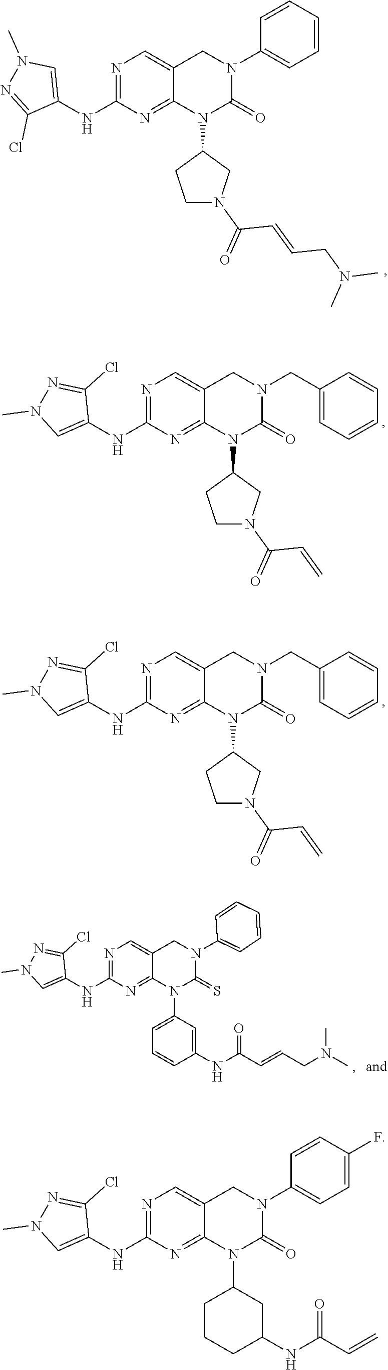

70. A pharmaceutical composition comprising a compound of any one of claims 1 to 69, or a pharmaceutically acceptable salt thereof, and a pharmaceutically acceptable carrier.

71. A method of inhibiting an epidermal growth factor receptor (EGFR) family kinase mutant in a subject in need thereof, comprising administering to the subject a therapeutically effective amount of a compound of any one of claims 1 to 69, or a pharmaceutically acceptable salt thereof.

72. A method of inhibiting a human epidermal growth factor receptor 2 (HER2) mutant in a subject in need thereof, comprising administering to the subject a therapeutically effective amount of a compound of any one of claims 1 to 69, or a pharmaceutically acceptable salt thereof.

73. The method of claim 72, wherein the HER2 mutant comprises an insertion in exon 20, an in-frame deletion and insertion in exon 20, a substitution in the extracellular domain, an extracellular truncation, or a substitution in exon 30.

74. The method of claim 73, wherein the HER2 mutant is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

75. A method of inhibiting an epidermal growth factor receptor (EGFR) mutant in a subject in need thereof, comprising administering to the subject a therapeutically effective amount of a compound of any one of claims 1 to 69, or a pharmaceutically acceptable salt thereof.

76. A method of inhibiting a drug-resistant epidermal growth factor receptor (EGFR) mutant in a subject in need thereof, comprising administering to the subject a therapeutically effective amount of a compound of any one of claims 1 to 69, or a pharmaceutically acceptable salt thereof.

77. The method of claim 76, wherein the drug-resistant EGFR mutant is del19/T790M EGFR or L858R/T790M EGFR.

78. A method of inhibiting human epidermal growth factor receptor 2 (HER2) in a subject in need thereof, comprising administering to the subject a therapeutically effective amount of a compound of any one of claims 1 to 69, or a pharmaceutically acceptable salt thereof, wherein the compound exhibits greater inhibition of a HER2 mutant relative to wild-type EGFR.

79. The method of claim 78, wherein the HER2 mutant comprises an insertion in exon 20, an in-frame deletion and insertion in exon 20, a substitution in the extracellular domain, an extracellular truncation, or a substitution in exon 30.

80. The method of claim 79, wherein the HER2 mutant is A775ins_G776insYVMA, A775_G776insSVMA, A775 G776insVVMA, G776del insVC, G776del insLC, G776del insAV, G776del insAVGC, S310F, S310Y, p95, V842I, or P780_Y781insGSP.

81. A method of inhibiting epidermal growth factor receptor (EGFR) in a subject in need thereof, comprising administering to the subject a therapeutically effective amount of a compound of any one of claims 1 to 69, or a pharmaceutically acceptable salt thereof, wherein the compound exhibits greater inhibition of an EGFR mutant relative to wild-type EGFR.

82. The method of claim 81, wherein the EGFR mutant comprises a substitution in exon 18, a deletion in exon 19, a substitution in exon 20, an insertion in exon 20, a mutation in the extracellular domain, or a substitution in exon 21.

83. The method of claim 82, wherein the EGFR mutant is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR.

84. The method of claim 83, wherein the EGFR mutant is del19/T790M EGFR or L858R/T790M EGFR.

85. A method of treating a disease or disorder associated with an epidermal growth factor receptor (EGFR) family kinase in a subject in need thereof, comprising administering to the subject a therapeutically effective amount of a compound of any one of claims 1 to 69, or a pharmaceutically acceptable salt thereof.

86. The method of claim 85, wherein the disease or disorder in the subject comprises a HER2 mutation.

87. The method of claim 86, wherein the HER2 mutation comprises an insertion in exon 20, an in-frame deletion and insertion in exon 20, a substitution in the extracellular domain, an extracellular truncation, or a substitution in exon 30.

88. The method of claim 87, wherein the HER2 mutation is A775ins_G776insYVMA, A775_G776insSVMA, A775 G776insVVMA, G776del insVC, G776del insLC, G776del insAV, G776del insAVGC, S310F, S310Y, p95, V842I, or P780_Y781insGSP.

89. The method of claim 85, wherein the disease or disorder in the subject comprises an EGFR mutation.

90. The method of claim 89, wherein the EGFR mutation comprises a substitution in exon 18, a deletion in exon 19, a substitution in exon 20, an insertion in exon 20, a mutation in the extracellular domain, or a substitution in exon 21.

91. The method of claim 90, wherein the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR.

92. The method of claim 91, wherein the EGFR mutation is del19/T790M EGFR or L858R/T790M EGFR.

93. A method of treating one or more cancer cells in a subject in need thereof, comprising administering to the subject a therapeutically effective amount of a compound of any one of claims 1 to 69, or a pharmaceutically acceptable salt thereof.

94. A method of treating cancer in a subject in need thereof, comprising administering to the subject a therapeutically effective amount of a compound of any one of claims 1 to 69, or a pharmaceutically acceptable salt thereof.

95. The method of claim 93 or 94, wherein the cancer is selected from bladder cancer, prostate cancer, breast cancer, cervical cancer, colorectal cancer, endometrial cancer, gastric cancer, glioblastoma, head and neck cancer, lung cancer, and non-small cell lung cancer.

96. The method of claim 95, wherein the cancer is selected from non-small cell lung cancer, prostate cancer, head and neck cancer, breast cancer, colorectal cancer, and glioblastoma.

97. The method of claim 93 or 94, wherein the cancer in the subject comprises a HER2 mutation.

98. The method of claim 97, wherein the HER2 mutation comprises an insertion in exon 20, an in-frame deletion and insertion in exon 20, a substitution in the extracellular domain, an extracellular truncation, or a substitution in exon 30.

99. The method of claim 98, wherein the HER2 mutation is A775ins_G776insYVMA, A775_G776insSVMA, A775_G776insVVMA, G776del insVC, G776del insLC, G776del insAV, G776del insAVGC, S310F, S310Y, p95, V842I, or P780_Y781insGSP.

100. The method of claim 93 or 94, wherein the cancer in the subject comprises an EGFR mutation.

101. The method of claim 100, wherein the EGFR mutation comprises a substitution in exon 18, a deletion in exon 19, a substitution in exon 20, an insertion in exon 20, a mutation in the extracellular domain, or a substitution in exon 21.

102. The method of claim 101, wherein the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR.

103. The method of claim 102, wherein the EGFR mutation is del19/T790M EGFR or L858R/T790M EGFR.

104. A method of treating an inflammatory disease in a subject in need thereof, comprising administering to the subject a therapeutically effective amount of a compound of any one of claims 1 to 69, or a pharmaceutically acceptable salt thereof.

105. The method of claim 104, wherein the inflammatory disease is selected from psoriasis, eczema, and atherosclerosis.

106. The method of claim 104, wherein the inflammatory disease in the subject comprises a HER2 mutation.

107. The method of claim 106, wherein the HER2 mutation comprises an insertion in exon 20, an in-frame deletion and insertion in exon 20, a substitution in the extracellular domain, an extracellular truncation, or a substitution in exon 30.

108. The method of claim 107, wherein the HER2 mutation is A775ins_G776insYVMA, A775_G776insSVMA, A775 G776insVVMA, G776del insVC, G776del insLC, G776del insAV, G776del insAVGC, S310F, S310Y, p95, V842I, or P780_Y781insGSP.

109. The method of claim 104, wherein the inflammatory disease in the subject comprises an EGFR mutation.

110. The method of claim 109, wherein the EGFR mutation comprises a substitution in exon 18, a deletion in exon 19, a substitution in exon 20, an insertion in exon 20, a mutation in the extracellular domain, or a substitution in exon 21.

111. The method of claim 110, wherein the EGFR mutation is del19/T790M EGFR, L858R/T790M EGFR, L858R EGFR, L861Q EGFR, G719X EGFR, 763insFQEA EGFR, 767insTLA EGFR, 769insASV EGFR, 769insGE EGFR, 770insSVD EGFR, 770insNPG EGFR, 770insGT EGFR, 770insGF EGFR, 770insG EGFR, 771insH EGFR, 771insN EGFR, 772insNP EGFR, 773insNPH EGFR, 773insH EGFR, 773insPH EGFR, EGFRvii, EGFRviii, D770_N771insSVD EGFR, H773insNPH EGFR, A767_dupASV EGFR, or 773insAH EGFR.

112. The method of claim 111, wherein the EGFR mutation is del19/T790M EGFR or L858R/T790M EGFR.
